(19) United States
(12) Patent Application Publication
(10) Pub. No.: US 2010/0120045 A1
(43) Pub. Date: May 13, 2010

(54) GENETIC VARIANTS USEFUL FOR RISK ASSESSMENTS OF CORONARY ARTERY DISEASE AND MYOCARDIAL INFARCTION

(75) Inventors: Anna Helgadottir, Reykjavik (IS); Gudmar Thorleifsson, Reykjavik (IS)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: DECODE GENETICS EHF, Reykjavik (IS)

(21) Appl. No.: 12/598,180

(22) PCT Filed: Apr. 30, 2008
(86) PCT No.: PCT/IS08/00011

§ 371 Date: Oct. 29, 2009

(30) Foreign Application Priority Data
Apr. 30, 2007 (IS) 8639

(51) Int. Cl. C12Q 1/68; C07H 21/00

(52) U.S. Cl. 435/6; 536/24.3

(57) ABSTRACT

The invention relates to methods of risk assessment and diagnosis of susceptibility to coronary artery disease and myocardial infarction, by assessing the presence or absence of alleles of certain polymorphic markers found to be associated with coronary artery disease and myocardial infarction. The invention also relates to methods for use of such polymorphic markers for predicting drug response to drugs for treating cardiovascular disease, or for monitoring the effectiveness of such drugs. The invention further relates to kits encompassing reagents for use in these methods.

### BACKGROUND OF THE INVENTION

### Epidemiology and Etiology

Cardiovascular diseases continue to be the principal cause of death in developed countries (Bonow, R O et al., Circulation 106:1602-05 (2002)). The costs of these diseases are high both in terms of morbidity and mortality, as well as in terms of the financial burden on health care systems. The major complications of coronary artery disease, i.e. Myocardial Infarction (MI) and Acute Coronary Syndrome (ACS), are the leading causes of hospital admissions in industrialized countries. Of all deaths caused by cardiovascular diseases, more than half are attributed to heart attack (myocardial infarction (MI)). The cost of the disease is high both in terms of morbidity and mortality, as well as in terms of the financial burden on the health care systems. According to the NHLBI's ARIC study (Thom, T et al., Circulation 113:e85-151 (2006)), the annual rate of first heart attack (MI or coronary heart disease (CHD) death) per 1000 individuals for ages 65-74 are 19.2 and 6.8, in European American men and women, respectively. For the same age group, the rates are 21.6 and 8.6, in African American men and women respectively. In the older age groups these numbers are much higher with the lifetime risk after the age of 40 of developing heart attack being 49% for men and 32% for women.

Myocardial infarction generally occurs when there is an abrupt decrease in coronary blood flow following a thrombotic occlusion of a coronary artery previously damaged by atherosclerosis (i.e. in subjects with coronary artery disease). In most cases, infarction occurs when an atherosclerotic plaque fissures, ruptures or ulcerates and when conditions favor thrombogenesis. In rare cases, infarction may be due to coronary artery occlusion caused by coronary emboli, congenital abnormalities, coronary spasm, and a wide variety of systemic, particularly inflammatory diseases. Medical risk factors for MI include cigarette smoking, diabetes, hypertension and serum total cholesterol levels >200 mg/dL, elevated serum LDL cholesterol, and low serum HDL cholesterol. Event rates in individuals without a prior history of cardiovascular disease are about 1%. In individuals who have had a first MI or ACS, the risk of a repeat MI within the next year is 10-14%, despite maximal medical management including angioplasty and stent placement.

Atherosclerosis can affect vascular beds in many large and medium arteries. Myocardial infarction and unstable angina (acute coronary syndrome (ACS)) stem from coronary artery atherosclerosis (coronary artery disease), while ischemic stroke most frequently is a consequence of carotid or cerebral artery atherosclerosis. Limb ischemia caused by peripheral arterial occlusive disease (PAOD) may occur as a consequence of iliac, femoral and popliteal artery atherosclerosis. The atherosclerotic diseases remain common despite the wide-spread use of medications that inhibit thrombosis (aspirin) or treat medical risk factors such as elevated cholesterol levels in blood (statins), diabetes, or hypertension (diuretics and antihypertensives).

Atherosclerotic disease is initiated by the accumulation of lipids within the artery wall, and in particular, the accumulation of low-density lipoprotein (LDL) cholesterol. The trapped LDL becomes oxidized and internalized by macrophages. This causes the formation of atherosclerotic lesions containing accumulations of cholesterol-engorged macrophages, referred to as “foam cells”. As disease progresses, smooth muscle cells proliferate and grow into the artery wall forming a “fibrous cap” of extracellular matrix enclosing a lipid-rich, necrotic core. Present in the arterial walls of most people throughout their lifetimes, fibrous atherosclerotic plaques are relatively stable. Such fibrous lesions cause extensive remodeling of the arterial wall, outwardly displacing the external, elastic membrane, without reduction in luminal diameter or serious impact on delivery of oxygen to the heart. Accordingly, patients can develop large, fibrous atherosclerotic lesions without luminal narrowing until late in the disease process. However, the coronary arterial lumen can become gradually narrowed over time and in some cases compromise blood flow to the heart, especially under high demand states such as exercise. This can result in reversible ischemia causing chest pain relieved by rest called stable angina.

In contrast to the relative stability of fibrous atherosclerotic lesions, the culprit lesions associated with myocardial infarction and unstable angina (each of which are part of the acute coronary syndrome) are characterized by a thin fibrous cap, a large lipid core, and infiltration of inflammatory cells such as T-lymphocytes and monocyte/macrophages. Non-invasive imaging techniques have shown that most MI's occur at sites with low- or intermediate-grade stenoses, indicating that coronary artery occlusion is due most frequently to rupture of culprit lesions with consequent formation of a thrombus or blood clot and not solely due to luminal narrowing by stenosis. Plaque rupture may be due to erosion or uneven thinning of the fibrous cap, usually at the margins of the lesion where macrophages enter, accumulate, and become activated by a local inflammatory process. Thinning of the fibrous cap may result from degradation of the extracellular matrix by proteases released from activated macrophages. These changes producing plaque instability and risk of MI may be augmented by production of tissue-factor procoagulant and other factors increasing the likelihood of thrombosis.

In acute coronary syndrome, the culprit lesion showing rupture or erosion with local thrombosis typically is treated by angioplasty or by balloon dilation and placement of a stent to maintain luminal patency. Patients experiencing ACS are at high risk for a second coronary event due to the multi-vessel nature of coronary artery disease with event rates approaching 10-14% within 12 months after the first incident.

The emerging view of MI is as an inflammatory disease of the arterial vessel wall on preexisting chronic atherosclerotic lesions, sometimes triggering rupture of culprit lesions and leading to local thrombosis and subsequent myocardial infarction. The process that triggers and sustains arterial wall inflammation leading to plaque instability is unknown, however, it results in the release into the circulation of tumor necrosis factor alpha and interleukin-6. These and other cytokines or biological mediators released from the damaged vessel wall stimulate an inflammatory response in the liver causing elevation in several non-specific general inflammatory markers including C-reactive protein. Although not specific to atherosclerosis, elevated C-reactive protein (CRP) and serum amyloid A appear to predict risk for MI, perhaps as surrogates for vessel wall inflammation. Many general inflammatory markers predict risk of coronary heart disease, although these markers are not specific to atherosclerosis. For example, Stein (Stein, S., Am J Cardiol, 87 (suppl):21A-26A (2001)) discusses the use of any one of the following serum inflammatory markers as surrogates for predicting risk of coronary heart disease including C-reactive protein (CRP), serum amyloid A, fibrinogen, interleukin-6, tissue necrosis factor-alpha, soluble vascular cell adhesion molecules (sVCAM), soluble intervascular adhesion molecules (sICAM), E-selectin, matrix metalloprotease type-1, matrix metalloprotease type-2, matrix metalloprotease type-3, and matrix metalloprotease type-9. Elevation in one more of these serum inflammatory markers is not specific to coronary heart disease but also occurs with age or in association with cerebrovascular disease, peripheral vascular disease, non-insulin dependent diabetes, osteoarthritis, bacterial infection, and sepsis. Elevated CRP or other serum inflammatory markers is also prognostic for increased risk of a second myocardial infarct in patients with a previous myocardial infarct (Retterstol, L. et al., Atheroscler., 160: 433-440 (2002)).

Although classical risk factors such as smoking, hyperlipidemia, hypertension, and diabetes are associated with many cases of coronary heart disease (CHD) and MI, many patients do not have involvement of these risk factors. In fact, many patients who exhibit one or more of these risk factors do not develop MI. Family history has long been recognized as one of the major risk factors. Although some of the familial clustering of MI reflects the genetic contribution to the other conventional risk factors, a large number of studies have suggested that there are significant genetic susceptibility factors, beyond those of the known risk factors (Friedlander Y, et al., Br. Heart J. 1985; 53:382-7, Shea S. et al., J. Am. Coll. Cardiol. 1984; 4:793-801, and Hopkins P. N., et al., Am. J. Cardiol. 1988; 62:703-7). Major genetic susceptibility factors have only been identified for the rare Mendelian forms of hyperlipidemia such as a familial hypercholesterolemia.

### Genetic Risk Factors

Genetic risk of disease is conferred by subtle differences in genes among individuals in a population. Genes differ between individuals most frequently due to single nucleotide polymorphisms (SNP), although other variations are also important. SNPs are located on average every 1000 base pairs in the human genome. Accordingly, a typical human gene containing 250,000 base pairs may contain 250 different SNPs. Only a minor number of SNPs are located in exons and alter the amino acid sequence of the protein encoded by the gene. Most SNPs have no effect on gene function, while others may alter transcription, splicing, translation, or stability of the mRNA encoded by the gene. Additional genetic polymorphisms in the human genome are caused by insertions, deletions, translocations, or inversions of either short or long stretches of DNA. Genetic polymorphisms conferring disease risk may therefore directly alter the amino acid sequence of proteins, may increase the amount of protein produced from the gene, or may decrease the amount of protein produced by the gene.

As genetic polymorphisms conferring risk of disease are uncovered, genetic testing for such risk factors is becoming important for clinical medicine. Examples are apolipoprotein E testing to identify genetic carriers of the ApoE4 polymorphism in dementia patients for the differential diagnosis of Alzheimer's disease, and of Factor V Leiden testing for predisposition to deep venous thrombosis. More importantly, in the treatment of cancer, diagnosis of genetic variants in tumor cells is used for the selection of the most appropriate treatment regime for the individual patient. In breast cancer, genetic variation in estrogen receptor expression or heregulin type 2 (Her2) receptor tyrosine kinase expression determine if anti-estrogenic drugs (tamoxifen) or anti-Her2 antibody (Herceptin) will be incorporated into the treatment plan. In chronic myeloid leukemia (CML) diagnosis of the Philadelphia chromosome genetic translocation fusing the genes encoding the Bcr and Abl receptor tyrosine kinases indicates that Gleevec (STI571), a specific inhibitor of the Bcr-Abl kinase should be used for treatment of the cancer. For CML patients with such a genetic alteration, inhibition of the Bcr-Abl kinase leads to rapid elimination of the tumor cells and remission from leukemia.

The familial clustering of CAD and MI has long been recognized and family history is considered one of the major risk factors for MI. Evidence for a genetic contribution to MI has been provided by both twin and family studies. These studies have estimated the heritability of MI to be 0.26 in males and 0.60 in females (Jorde, L. et al. Medical Genetics 3^{rd} ed. St. Louis: Mosby (2003)) and that the concordance rates for monzygotic vs. dizygotic twins is 0.39 vs. 0.26 for males and 0.44 vs. 0.14 for females. A longitudinal study of 21,004 Swedish twins showed that the relative hazard of death from CAD when one twin died of an early-onset CAD (defined as before age of 55 for males and 65 for females), compared to the hazard ratio when one's twin did not die of early-onset CAD, was 8.1 and 3.8 for male monozygotic and dizygotic twins, respectively (Marenberg, Me., N Engl J Med 330:1041-46 (1994)). The corresponding relative hazard for female twins was 15 for monozygotic twins and 2.6 for dizygotic twins.

Family studies have consistently demonstrated that having a first degree relative with premature CAD death increases the risk of developing CAD (Schildkraut, 3M et al Am J Cardiol 64:555-59 (1989); Slack, 3 & Evans, K A, J Med Genet 3:239-57 (1966)), although the reported increase in risk has varied between studies. The mechanisms/processes by which the family history contributes to MI are currently not well defined. Some of the familial clustering of MI may reflect the familial aggregation to the other conventional risk factors. However, a large number of studies, including the Reykjavik Cohort Study, have suggested that there are significant genetic susceptibility factors, beyond those of the known risk factors (Friedlander, Y., Br Heart J 53:382-87 (1985); Shea, S., J Am Coll Cardiol 4:793-801 (1984); Andresdottir, M B, Eur Heart J 23:1655-63 (2002)). The Reykjavik Cohort Study in Iceland examined the relationship between the history of MI in first-degree relatives and the risk of developing CHD (MI or coronary revascularization) in a large prospective study. Compared with subjects without a family history, the hazard ratio of CHD was 1.75 (95% CI: 1.59-1.92) for men and 1.83 (1.60-2.11) for women, with one or more first-degree relatives with MI, and after adjusting for the conventional risk factors, it was 1.66 (1.51-1.82) and 1.64 (1.43-1.89) for men and women, respectively (Andresdottir, M B, Eur Heart J 23:1655-63 (2002)).

Case-control association studies have suggested the involvement of specific genetic susceptibility variants for MI. The majority of those studies have focused on variants in selected candidate genes (the candidate gene approach), genes that might be involved in the pathogenesis of the disease, or in the development of the intermediate phenotype (e.g. known risk factors). In most cases SNPs that change amino acids or are in promoter regions of genes, have been tested for association. Two studies have tested variants within multiple candidate genes for association with MI. In one, association of C1019T polymorphism in the connexin 37 gene to MI in men and 4G-668-5G polymorphism in the plasminogen-activator-inhibitor type 1 gene and the 5A-1171/6A polymorphism in the stromelysin-1 gene in women Was reported (Yamada, Y. et al N Engl J Med 347:1916-23 (2002)). Another study examining 72 SNPs in 62 candidate genes in individuals with premature MI and controls from the US, showed that three variants in genes encoding thrombosponding-4, thrombospondin-2, and thrombospondin-1, associated with premature MI (Topol, E J, et al Circulation 104:2641-44 (2001). A more recent study tested 11,053 SNPs in 6,891 genes for association to MI in a group of European-Americans (Shiffman, d., et al Am J Hum Genet 77:596-605 (2005)). The SNPs tested were primarily selected as variants likely to affect protein function or expression. MI associated variants in four novel genes (KIAA0992, ROS1, TAS2R50, and OR13G1) were identified through this effort. Importantly, variants identified as associated to MI in the above studies remain to be validated in replication studies. Their involvement in the etiology of MI therefore remains uncertain.

Recently, genetic susceptibility variants for CAD and MI were identified on chromosome 9, within a region that contains the CDKN2A and CDKN2B genes (Helgadottir, A. et al, Science 316:1491-93 (2007); McPherson, R. et al, Science 316:1488-91 (2007); The Wellcome Trust Case Control Consortium, Nature 447:661-78 (2007); Samani, N J et al, N Engl J Med 357:443-53 (2007)). Follow-up investigations have shown that the variants, which are all located within a region of high linkage disequilibrium (LD), also contribute to risk of intracranial aneurysm and abdominal aortic aneurysm (Helgadottir, A. et al, Nature Genet 40:217-24 (2008)), showing that these variants not only contribute to MI and CAD, but also to cardiovascular disease in general.

Cardiovascular disease remains the most important cause or morbidity and mortality and there is a large unmet medical need for its prevention. Identifying the key pathways involved in MI and CAD may facilitate development of new therapeutics and diagnostics. Identification of genetic risk factors for MI and CAD may offer clinical practitioners valuable diagnostic and prognostic tools. Genetic variants that are found to be an important key to the presentation of clinical symptoms in MI may also facilitate drug development, through the identification of novel drug targets.

There is thus an unmet need for identification of additional genetic variants that confer susceptibility of CAD and MI. The present invention provides such variants.

### SUMMARY OF THE INVENTION

The present invention relates to methods of risk assessment, diagnosis and/or prognosis of coronary artery disease (CAD) and myocardial infarction (MI). Certain genetic variants have been found to be associated with risk of CAD and/or MI. The invention thus relates to methods of assessing increased susceptibility to CAD and/or MI, as well as methods of assessing a decreased susceptibility to CAD and/or MI, or determine a protection against CAD and/or MI. The methods of the invention relate to the evaluation of certain polymorphic markers, as described herein, that have been found to be useful in disease prediction and management, through their association with susceptibility of coronary artery disease and myocardial infarction.

In one aspect, the invention relates to a method for determining a susceptibility to coronary artery disease or myocardial infarction in a human individual, comprising determining the presence or absence of at least one allele of at least one polymorphic marker in a nucleic acid sample obtained from the individual or in a genotype dataset derived from the individual, wherein the at least one polymorphic marker is selected from the polymorphic markers set forth in Table 4, and markers in linkage disequilibrium therewith, and wherein the presence of the at least one allele is indicative of a susceptibility to coronary artery disease or myocardial infarction. The method may in one embodiment relate to determination of the presence or absence of at least one allele of at least one polymorphic marker in a nucleic acid sample obtained from the individual. In another embodiment, the method relates to determination of the presence or absence of at least one allele of at least one polymorphic marker in a genotype dataset derived from the individual. The genotype dataset can be derived from the individual in the sense that the information that it contains (genotype information) relates to a particular nucleic acid sample as a template from a single individual, for whom genetic information is derived.

In another aspect, the present invention relates to a method of determining a susceptibility to coronary artery disease or myocardial infarction in a human individual, comprising determining the presence or absence of at least one allele of at least one polymorphic marker in a nucleic acid sample obtained from the individual, wherein the at least one polymorphic marker is selected from rs11751605 (SEQ ID NO:1), and markers in linkage disequilibrium therewith, wherein determination of the presence or absence of the at least one allele is indicative of a susceptibility to coronary artery disease or myocardial infarction. In one embodiment, the at least one polymorphic marker is selected from the markers set forth in Table 5, and markers in linkage disequilibrium therewith.

In an alternative aspect, the invention relates to a method of diagnosing a susceptibility to coronary artery disease or myocardial infarction in a human individual, the method comprising determining the presence or absence of at least one allele of at least one polymorphic marker in a nucleic acid sample obtained from the individual, or in a genotype dataset derived from the individual, wherein the at least one polymorphic marker is selected from rs11751605 (SEQ ID NO:1), and markers in linkage disequilibrium therewith, wherein the presence of the at least one allele is indicative of a susceptibility to coronary artery disease or myocardial infarction.

In another aspect, the invention relates to a method of determining a susceptibility to coronary artery disease or myocardial infarction in a human individual, comprising determining whether at least one at-risk allele in at least one polymorphic marker is present in a genotype dataset derived from the individual, wherein the at least one polymorphic marker is selected from the markers set forth in Table 4, and markers in linkage disequilibrium therewith, and wherein determination of the presence of the at least one at-risk allele is indicative of increased susceptibility to coronary artery disease or myocardial infarction in the individual.

The genotype dataset comprises in one embodiment information about marker identity, and the allelic status of the individual, i.e. information about the identity of the two alleles carried by the individual for the marker. The genotype dataset may comprise allelic information about one or more marker, including two or more markers, three or more markers, five or more markers, one hundred or more markers, etc. In some embodiments, the genotype dataset comprises genotype information from a whole-genome assessment of the individual including hundreds of thousands of markers, or even one million or more markers.

In certain embodiments, the at least one polymorphic marker is selected from the markers set forth in Table 4. In certain other embodiments, the at least one polymorphic marker is associated with the Lipoprotein (a) gene. In certain other embodiments, the at least one polymorphic marker is selected from rs11751605, rs6076623, rs1412444, rs2163612, rs1029396, rs2243547, rs12534186, rs12134779, rs7158073, rs254850, rs2417821, rs7661204, rs4921437, rs832540, rs324594, rs1741318, rs9902941, rs7709212, rs2946534, rs6556861, rs8050136, rs3751812, rs4769613, rs2243548, rs12459084, rs2074464, rs2244871, rs270654, rs854787, rs7944761, rs4779984, rs6502622, rs3183702, rs1433048, rs4925119, rs2476601, rs870347, rs334198, rs854813, rs7753765, rs4925114, rs270661, rs953861, rs10045431, rs8003722, rs2297538, rs12329252, rs3748744, rs4704400, rs3102526, rs2110209, rs1870843 and rs3134517.

In one embodiment, the method of determining a susceptibility, or diagnosing a susceptibility of, coronary artery disease or myocardial infarction, further comprises assessing the frequency of at least one haplotype in the individual. In one such embodiment, the at least one haplotype is selected from haplotypes that are in linkage disequilibrium with at least one marker as set forth in Table 4. In another embodiment, the at least one haplotype is selected from the haplotypes that are in linkage disequilibrium with rs11751605 (SEQ ID NO:1).

In certain embodiments of the invention, determination of the presence of at least one at-risk allele of at least one polymorphic Marker in a nucleic acid sample from the individual is indicative of an increased susceptibility to coronary artery disease or myocardial infarction. In one embodiment, the increased susceptibility is characterized by a relative risk (RR) or odds ratio (OR) of at least 1.15. In another embodiment, the increased susceptibility is characterized by a relative risk (RR) or odds ratio (OR) of at least 1.20.

In some embodiments, the presence of allele C in rs11751605, allele T in rs6076623, allele A in rs1412444, allele G in rs2163612 or allele G in rs1029396 is indicative of increased susceptibility of coronary artery disease or myocardial infarction in the individual.

In some other embodiments, the presence of at least one protective allele in a nucleic acid sample from the individual is indicative of a decreased susceptibility of coronary artery disease or myocardial infarction. In another embodiment, the absence of at least one at-risk allele in a nucleic acid sample from the individual is indicative of a decreased susceptibility of coronary artery disease or myocardial infarction.

Another aspect of the invention relates to a method of assessing a susceptibility to coronary artery disease or myocardial infarction in a human individual, comprising screening a nucleic acid from the individual for at least one polymorphic marker or haplotype in the genomic segment with the sequence as set forth in any one of SEQ ID NO:1-172 that correlates with increased occurrence of coronary artery disease or myocardial infarction in a human population, wherein the presence of an at-risk marker allele in the at least one polymorphism or an at-risk haplotype in the nucleic acid identifies the individual as having elevated susceptibility to coronary artery disease or myocardial infarction, and wherein the absence of the at least one at-risk marker allele or at-risk haplotype in the nucleic acid identifies the individual as not having the elevated susceptibility.

In one such embodiment, the at least one polymorphic marker or haplotype comprises at least one polymorphic marker selected from the markers set forth in Table 4, and polymorphic markers in linkage disequilibrium therewith.

Certain embodiments of the invention further comprise a step of screening the nucleic acid for the presence of at least one at-risk genetic variant for a coronary artery disease or myocardial infarction not associated with, i.e. not in linkage disequilibrium with, any one of the markers set forth in Table 4. Such additional genetic variants can in specific embodiments include any variant that has been identified as a susceptibility or risk variant for coronary artery disease or myocardial infarction.

In another aspect of the present invention, the presence of the marker or haplotype found to be associated with coronary artery disease or myocardial infarction, and as such useful for determining a susceptibility to coronary artery disease or myocardial infarction, is indicative of a different response rate of the subject to a particular treatment modality for coronary artery disease or myocardial infarction.

In another aspect, the invention relates to a method of identification of a marker for use in assessing susceptibility to coronary artery disease or myocardial infarction in human individuals, the method comprising:
identifying at least one polymorphic marker in linkage disequilibrium with at least one of the markers set forth in Table 4;
determining the genotype status of a sample of individuals diagnosed with, or having a susceptibility to, coronary artery disease or myocardial infarction; and
determining the genotype status of a sample of control individuals;
   wherein a significant difference in frequency of at least one allele in at least one polymorphism in individuals diagnosed with, or having a susceptibility to, coronary artery disease or myocardial infarction, as compared with the frequency of the at least one allele in the control sample is indicative of the at least one polymorphism being useful for assessing susceptibility to coronary artery disease or myocardial infarction.

In one embodiment, “significant” is determined by statistical means, e.g. the difference is statistically significant. In one such embodiment, statistical significance is characterized by a P-value of less than 0.05. In other embodiments, the statistical significance is characterized a P-value of less than 0.01, less than 0.001, less than 0.0001, less than 0.00001, less than 0.000001, less than 0.0000001, less than 0.0000000001, or less than 0.00000001.

In one embodiment, an increase in frequency of the at least one allele in the at least one polymorphism in individuals diagnosed with, or having a susceptibility to, coronary artery disease or myocardial infarction, as compared with the frequency of the at least one allele in the control sample, is indicative of the at least one polymorphism being useful for assessing increased susceptibility to coronary artery disease or myocardial infarction. In another embodiment, a decrease in frequency of the at least one allele in the at least one polymorphism in individuals diagnosed with, or having a susceptibility to, a coronary artery disease or myocardial infarction, as compared with the frequency of the at least one allele in the control sample is indicative of the at least one polymorphism being useful for assessing decreased susceptibility to, or protection against, the coronary artery disease or myocardial infarction.

Another aspect of the invention relates to a method of genotyping a nucleic acid sample obtained from a human individual, comprising determining the presence or absence of at least one allele of at least one polymorphic marker in the sample, wherein the at least one marker is selected from the markers set forth in Table 4, and markers in linkage disequilibrium therewith, and wherein determination of the presence or absence of the at least one allele of the at least one polymorphic marker is predictive of a susceptibility of coronary artery disease or myocardial infarction.

In one embodiment, genotyping comprises amplifying a segment of a nucleic acid that comprises the at least one polymorphic marker by Polymerase Chain Reaction (PCR), using a nucleotide primer pair flanking the at least one polymorphic marker. In another embodiment, genotyping is performed using a process selected from allele-specific probe hybridization, allele-specific primer extension, allele-specific amplification, nucleic acid sequencing, 5′-exonuclease digestion, molecular beacon assay, oligonucleotide ligation assay, size analysis, and single-stranded conformation analysis. In one particular embodiment, the process comprises allele-specific probe hybridization. In another embodiment, the process comprises DNA sequencing. In a preferred embodiment, the method comprises:
1) contacting copies of the nucleic acid with a detection oligonucleotide probe and an enhancer oligonucleotide probe under conditions for specific hybridization of the oligonucleotide probe with the nucleic acid;
   wherein
   a) the detection oligonucleotide probe is from 5-100 nucleotides in length and specifically hybridizes to a first segment of the nucleic add whose nucleotide sequence is given by any one of SEQ ID NO:1-172 that comprises at least one polymorphic site;
   b) the detection oligonucleotide probe comprises a detectable label at its 3′ terminus and a quenching moiety at its 5′ terminus;
   c) the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; and
   d) a single base gap exists between the first segment and the second segment, such that when the oligonucleotide probe and the enhancer oligonucleotide probe are both hybridized to the nucleic acid, a single base gap exists between the oligonucleotides;
2) treating the nucleic acid with an endonuclease that will cleave the detectable label from the 3′ terminus of the detection probe to release free detectable label when the detection probe is hybridized to the nucleic acid; and
3) measuring free detectable label, wherein the presence of the free detectable label indicates that the detection probe specifically hybridizes to the first segment of the nucleic acid, and indicates the sequence of the polymorphic site as the complement of the detection probe.

In a particular embodiment, the copies of the nucleic acid are provided by amplification by Polymerase Chain Reaction (PCR). In another embodiment, the susceptibility determined is increased susceptibility. In another embodiment, the susceptibility determined is decreased susceptibility.

Another aspect of the invention relates to a method of assessing an individual for probability of response to a therapeutic agent for preventing and/or ameliorating symptoms associated with coronary artery disease or myocardial infarction, comprising: determining the presence or absence of at least one allele of at least one polymorphic marker in a nucleic acid sample obtained from the individual, wherein the at least one polymorphic marker is selected from the markers set forth in Table 4, and markers in linkage disequilibrium therewith, wherein determination of the presence of the at least one allele of the at least one marker is indicative of a probability of a positive response to coronary artery disease or myocardial infarction therapeutic agent. In one embodiment, the at least one polymorphic marker is selected from marker rs11751605, rs6076623, rs1412444, rs2163612 and rs1029396, and markers in linkage disequilibrium therewith. In one embodiment, the therapeutic agent is selected from beta blockers, anticoagulation agents, including heparin and/or low molecular weigh heparin, antiplatelet agents, such as clopidogrel, aspirin, beta blockers, including metoprolol and carvedilol, ACE inhibitors, Statins, Aldosterone antagonists, including eplerenone, leukotriene synthesis inhibitors, the agents set forth in Agent Table I, Agent Table II, (R)-(+)-alpha-cyclopentyl-4-(2-quinolinylmethoxy)-Benzeneacetic acid, atreleuton, and 4-{(S)-2-[4-(4-Chloro-phenoxy)-phenoxymethyl]-pyrrolidin-1-yl}-butyramide, also known as DG-051. Other embodiments may include any one or a combination of the therapeutic agents described herein to be useful for therapeutic intervention of coronary artery disease or myocardial infarction.

Yet another aspect of the invention relates to a method of predicting prognosis of an individual diagnosed with coronary artery disease or myocardial infarction, the method comprising determining the presence or absence of at least one allele of at least one polymorphic marker in a nucleic acid sample obtained from the individual, wherein the at least one polymorphic marker is selected from the group consisting of the markers set forth in Table 3, and markers in linkage disequilibrium therewith, wherein determination of the presence of the at least one allele is indicative of a worse prognosis of coronary artery disease or myocardial infarction in the individual. The prognosis may in certain embodiment relate to susceptibility of recurrent MI events, recurrent stroke events, or susceptibility to other complications relating to coronary artery disease or myocardial infarction.

A further aspect of the invention relates to a method of monitoring progress of a treatment of an individual undergoing treatment for coronary artery disease or myocardial infarction, the method comprising determining the presence or absence of at least one allele of at least one polymorphic marker in a nucleic acid sample obtained from the individual, wherein the at least one polymorphic marker is selected from the group consisting of the markers set forth in Table 3, and markers in linkage disequilibrium therewith, wherein determination of the presence of the at least one allele is indicative of the treatment outcome of the individual. The treatment may in certain embodiments be surgical treatment. In other embodiments, the treatment is by administration of a therapeutic agent, optionally including lifestyle changes or alterations in environmental exposure to risk factors for coronary artery disease or myocardial infarction, as described further herein.

In one embodiment, the method further comprises assessing at least one biomarker in a sample from the individual. The biomarker is in certain embodiments a cardiac marker or an inflammatory marker. In one embodiment, the at least one biomarker is selected from creatin kinase, troponin, glycogen phosphorylase, C-reactive protein (CRP), serum amyloid A, fibrinogen, interleukin-6, tissue necrosis factor-alpha, soluble vascular cell adhesion molecules (sVCAM), soluble intervascular adhesion molecules (sICAM), E-selectin, matrix metalloprotease type-1, matrix metalloprotease type-2, matrix metalloprotease type-3, matrix metalloprotease type-9, serum sCD40L, leukotrienes, leukotriene metabolites, interleukin-6, tissue necrosis factor-alpha, myeloperoxidase (MPO), and N-tyrosine. In one embodiment, the leukotriene is selected from LTB4, LTC4, LTD4 and LTE4. In another embodiment, the method further comprises analyzing non-genetic information to make risk assessment, diagnosis, or prognosis of the individual. The non-genetic information is in one embodiment selected from age, gender, ethnicity, socioeconomic status, previous disease diagnosis, medical history of subject, family history of coronary artery disease or myocardial infarction, biochemical measurements, and clinical measurements. In a particular preferred embodiment, a further step comprising calculating overall risk is employed.

Another aspect of the invention relates to analyzing a sample comprising genomic DNA from a human individual or a genotype dataset derived from a human individual for the presence or absence of at least one at-risk allele of at least one at-risk variant for coronary artery disease or myocardial infarction not in linkage disequilibrium with any one of the markers set forth in Table 4. Thus, the variants described herein to be associated with coronary artery disease or myocardial infarction may be combined with other genetic variants for coronary artery disease or myocardial infarction, that are not genetically related (i.e., not in linkage disequilibrium with) the markers described herein. Such analysis may be undertaken in combination with any of the methods described herein. Furthermore any two markers herein, or any other combination of markers and/or haplotypes described herein to be associated with coronary artery disease or myocardial infarction may be combined to assess an increased susceptibility to coronary artery disease or myocardial infarction.

In some embodiments of the methods of the invention, non-genetic information is analyzed, to make risk assessment, diagnosis, or prognosis of the individual. The non-genetic information is in certain embodiments selected from age, gender, ethnicity, socioeconomic status, previous disease diagnosis, medical history of subject, family history of coronary artery disease or myocardial infarction, biochemical measurements, and clinical measurements. Combined genetic factors and/or combinations of genetic and non-genetic factors may be analyzed by known methods, to generate a combined risk.

The invention also relates to a kit for assessing susceptibility to a coronary artery disease or myocardial infarction in a human individual, the kit comprising reagents for selectively detecting the presence or absence of at least one allele of at least one polymorphic marker in the genome of the individual, wherein the polymorphic marker is selected from the markers set forth in Table 3, and markers in linkage disequilibrium therewith, and wherein the presence of the at least one allele is indicative of a susceptibility to a coronary artery disease or myocardial infarctions.

In one embodiment, the reagents comprise at least one contiguous oligonucleotide that hybridizes to a fragment of the genome of the individual comprising the at least one polymorphic marker, a buffer and a detectable label. In one embodiment, the reagents comprise at least one pair of oligonucleotides that hybridize to opposite strands of a genomic nucleic acid segment obtained from the subject, wherein each oligonucleotide primer pair is designed to selectively amplify a fragment of the genome of the individual that includes one polymorphic marker, and wherein the fragment is at least 30 base pairs in size. In a particular embodiment the at least one oligonucleotide is completely complementary to the genome of the individual. In another embodiment, the at least one oligonucleotide can comprise at least one mismatch to the genome of the individual. In one embodiment, the oligonucleotide is about 18 to about 50 nucleotides in length. In another embodiment, the oligonucleotide is 20-30 nucleotides in length.

In one preferred embodiment, the kit comprises:

a detection oligonucleotide probe that is from 5-100 nucleotides in length; an enhancer oligonucleotide probe that is from 5-100 nucleotides in length; and an endonuclease enzyme;
wherein the detection oligonucleotide probe specifically hybridizes to a first segment of the nucleic acid whose nucleotide sequence is given by any one of SEQ ID NO:1-172 that comprises at least one polymorphic site; and wherein the detection oligonucleotide probe comprises a detectable label at its 3′ terminus and a quenching moiety at its 5′ terminus; wherein the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; wherein a single base gap exists between the first segment and the second segment, such that when the oligonucleotide probe and the enhancer oligonucleotide probe are both hybridized to the nucleic acid, a single base gap exists between the oligonucleotides; and wherein treating the nucleic acid with the endonuclease will cleave the detectable label from the 3′ terminus of the detection probe to release free detectable label when the detection probe is hybridized to the nucleic acid.

A further aspect of the invention relates to the use of an oligonucleotide probe in the manufacture of a diagnostic reagent for diagnosing and/or assessing susceptibility to coronary artery disease or myocardial infarction in a human individual, wherein the probe hybridizes to a segment of a nucleic acid whose nucleotide sequence is given by any one of SEQ ID NO:1-172 that comprises at least one polymorphic site, wherein the fragment is 15-500 nucleotides in length. In one embodiment, the polymorphic site is selected from the polymorphic markers rs11751605, rs6076623, rs1412444, rs2163612 and rs1029396, and markers in linkage disequilibrium therewith

Yet another aspect of the invention relates to a computer-readable medium on which is stored: an identifier for at least one polymorphic marker; an indicator of the frequency of at least one allele of said at least one polymorphic marker in a plurality of individuals diagnosed with a coronary artery disease or myocardial infarction; and an indicator of the frequency of the least one allele of said at least one polymorphic markers in a plurality of reference individuals; wherein the at least one polymorphic marker is selected from the polymorphic markers set forth in Table 4, and markers in linkage disequilibrium therewith. In one embodiment, the at least one polymorphic marker is selected from rs11751605, rs6076623, rs1412444, rs2163612, rs1029396, and markers in linkage disequilibrium therewith.

Another aspect relates to an apparatus for determining a genetic indicator for coronary artery disease or myocardial infarction in a human individual, comprising: a computer readable memory; and a routine stored on the computer readable memory; wherein the routine is adapted to be executed on a processor to analyze marker and/or haplotype information for at least one human individual with respect to at least one polymorphic marker selected from the markers set forth in Table 4, and markers in linkage disequilibrium therewith, and generate an output based on the marker or haplotype information, wherein the output comprises a risk measure of the at least one marker or haplotype as a genetic indicator of coronary artery disease or myocardial infarction for the human individual.

In one embodiment, the routine further comprises an indicator of the frequency of at least one allele of at least one polymorphic marker or at least one haplotype in a plurality of individuals diagnosed with coronary artery disease or myocardial infarction, and an indicator of the frequency of at the least one allele of at least one polymorphic marker or at least one haplotype in a plurality of reference individuals, and wherein a risk measure is based on a comparison of the at least one marker and/or haplotype status for the human individual to the indicator of the frequency of the at least one marker and/or haplotype information for the plurality of individuals diagnosed with coronary artery disease or myocardial infarction.

The present invention, as described herein, may be reduced to practice using any one, or a combination of, the polymorphic markers described herein as being useful for the determination of a susceptibility to coronary artery disease or myocardial infarction. This includes markers that are shown herein to be associated with coronary artery disease or myocardial infarction, but also includes markers that are in linkage disequilibrium with such variants. In one embodiment, the at least one marker is selected from the markers set forth in Table 3. In another embodiment, the at least one marker is selected from the markers set forth in Table 4. In another embodiment, the at least one marker is selected from rs11751605 (SEQ ID NO:1), rs6076623 (SEQ ID NO:2), rs1412444 (SEQ ID NO:3), rs2163612 (SEQ ID NO:4), rs1029396 (SEQ ID NO:5), rs2243547 (SEQ ID NO:6), rs12534186 (SEQ ID NO:7), rs12134779 (SEQ ID NO:8), rs7158073 (SEQ ID NO:9), rs254850 (SEQ ID NO:10), rs2417821 (SEQ ID NO:11), rs7661204 (SEQ ID NO:12), rs4921437 (SEQ ID NO:13), rs832540 (SEQ ID NO:14), rs324594 (SEQ ID NO:15), rs1741318 (SEQ ID NO:16), rs9902941 (SEQ ID NO:17), rs7709212 (SEQ ID NO:18), rs2946534 (SEQ ID NO:19), rs6556861 (SEQ ID NO:20), rs8050136 (SEQ ID NO:21), rs3751812 (SEQ ID NO:22), rs4769613 (SEQ ID NO:23), rs2243548 (SEQ ID NO:24), rs12459084 (SEQ ID NO:25), rs2074464 (SEQ ID NO:26), rs2244871 (SEQ ID NO:27), rs270654 (SEQ ID NO:28), rs854787 (SEQ ID NO:29), rs7944761 (SEQ ID NO:30), rs4779984 (SEQ ID NO:31), rs6502622 (SEQ ID NO:32), rs3183702 (SEQ ID NO:33), rs1433048 (SEQ ID NO:34), rs4925119 (SEQ ID NO:35), rs2476601 (SEQ ID NO:36), rs870347 (SEQ ID NO:37), rs334198 (SEQ ID NO:38), rs854813 (SEQ ID NO:39), rs7753765 (SEQ ID NO:40), rs4925114 (SEQ ID NO:41), rs270661 (SEQ ID NO:42), rs953861 (SEQ ID NO:43), rs10045431 (SEQ ID NO:44), rs8003722 (SEQ ID NO:45), rs2297538 (SEQ ID NO:46), rs12329252 (SEQ ID NO:47), rs3748744 (SEQ ID NO:48), rs4704400 (SEQ ID NO:49), rs3102526 (SEQ ID NO:50), rs2110209 (SEQ ID NO:51), rs1870843 (SEQ ID NO:52) and rs3134517 (SEQ ID NO:53). In another embodiment, the at least one marker is selected from rs11751605 (SEQ ID NO:1), rs6076623 (SEQ ID NO:2), rs1412444 (SEQ ID NO:3), rs2163612 (SEQ ID NO:4), rs1029396 (SEQ ID NO:5). In another embodiment, the at least one marker is selected from rs11751605 (SEQ ID NO:1), rs6076623 (SEQ ID NO:2), rs1412444 (SEQ ID NO:3), rs2163612 (SEQ ID NO:4), rs1029396 (SEQ ID NO:5), rs2243547 (SEQ ID NO:6), rs12534186 (SEQ ID NO:7), rs12134779 (SEQ ID NO:8), rs7158073 (SEQ ID NO:9). In another embodiment, the at least one marker is rs11751605 (SEQ ID NO:1). In another embodiment, the at least one marker is rs6076623 (SEQ ID NO:2). In another embodiment, the at least one marker is rs1412444 (SEQ ID NO:3). In another embodiment, the at least one marker is rs2163612 (SEQ ID NO:4). In another embodiment, the at least one marker is rs1029396 (SEQ ID NO:5). In these embodiments, the at least one marker may optionally be selected from markers in linkage disequilibrium with the at least one marker.

The present invention relates to methods, uses, apparatus or kits useful for detecting or determining a susceptibility to myocardial infarction or coronary artery disease. It is however contemplated that the variants described herein to be correlated with risk of developing myocardial infarction or coronary artery disease may also be risk variants for other cardiovascular diseases. This includes Stroke, Peripheral Artery Disease, Restenosis, Intracranial Aneurysm and Aorta Abdominal Aneurysm. The Restenosis phenotype may for example be Coronary In-stent Restenosis. The In-stent Restenosis may further be either Restenosis following Bare Metal Stent (BMS) placement, or Restenosis following placement of a Drug Eluting Stent (DES).

Variants (markers and/or haplotypes comprising polymorphic markers) in linkage disequilibrium with the markers and haplotypes of the present invention are also useful for the methods and kits of the invention. The invention therefore also pertains to markers in linkage disequilibrium with the markers and haplotypes of the invention. In certain embodiments of the methods, uses, apparatus or kits of the invention, linkage disequilibrium is characterized by specific cutoff values for a quantitative measure of linkage disequilibrium. In one such embodiment, linkage disequilibrium is characterized by specific numerical values, or ranges of numerical values, for r². In another such embodiment, linkage disequilibrium is characterized by specific numerical values, or ranges of numerical values, for |D′|. In yet another embodiment, linkage disequilibrium is characterized by specific numerical values for r² and |D′|, or ranges thereof. In one preferred embodiment, linkage disequilibrium is characterized by values for r² of greater than 0.1. In another preferred embodiment, linkage disequilibrium is characterized by values for r² of greater than 0.2. Other cutoff values for r² are also possible, including, but not limited to, 0.3, 0.4, 0.5, 0.6, 0.7, 0.8, 0.9, 0.95, 0.96, 0.97, 0.98, 0.99. In another preferred embodiment, linkage disequilibrium is characterized by values for |D′| of greater than 0.5. In another preferred embodiment, linkage disequilibrium is characterized by values for |D′| of greater than 0.8. Other cutoff values for |D′| are also possible, including, but not limited to, 0.2, 0.3, 0.4, 0.6, 0.7, 0.8, 0.9, 0.95, 0.96, 0.97, 0.98 and 0.99. In certain embodiments, linkage disequilibrium is characterized by numeric cutoff values for either |D′| and r². In one such embodiment linkage disequilibrium is characterized by numerical values for either |D′| of greater than 0.8 and r² of greater than 0.2, or both.

In certain other embodiments of the methods, uses, apparatus or kits of the invention, the individual is of a specific human ancestry. In one embodiment, the ancestry is selected from black African ancestry, Caucasian ancestry and Chinese ancestry. In another embodiment, the ancestry is black African ancestry. In another embodiment, the ancestry is African American ancestry. In another embodiment, the ancestry is European ancestry. In another embodiment, the ancestry is Caucasian ancestry. The ancestry is in certain embodiment self-reported by the individual who undergoes genetic analysis or genotyping. In other embodiments, the ancestry is determined by genetic determination comprising detecting at least one allele of at least one polymorphic marker in a nucleic acid sample from the individual, wherein the presence or absence of the allele is indicative of the ancestry of the individual.

In other particular other embodiments of the methods, uses, apparatus or kits of the invention, the presence of at least one at-risk variant, i.e. an at-risk allele in at least one polymorphic marker or an at-risk haplotype, is indicative of an early onset of coronary artery disease or myocardial infarction. Early onset is in some embodiments categorized as onset before age 75. In other embodiments, early onset is categorized as onset before age 70, before age 65, before age 60, before age 55, before age 50, before age 45, or before age 40. Other values for categorization of age at onset are also contemplated, including, but not limited to, all integer values of age, and such age categories are also within scope of the invention. In certain embodiments, the age at onset is below 50 for males and/or below 60 for females.

### DETAILED DESCRIPTION OF THE INVENTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, microsatellites, insertions, deletions, duplications and translocations.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d′Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 by longer than the shorter allele in the CEPH sample, allele 2 is 2 by longer than the shorter allele in the CEPH sample, allele 3 is 3 by longer than the lower allele in the CEPH sample, etc., and allele −1 is 1 by shorter than the shorter allele in the CEPH sample, allele −2 is 2 by shorter than the shorter allele in the CEPH sample, etc.

Sequence conucleotide ambiguity as described herein is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C G or T |
| D | A G or T |
| H | A C or T |
| V | A C or G |
| N | A C G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles.

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular polymorphic markers and/or haplotypes of the invention may be characteristic of increased susceptibility (i.e., increased risk) of coronary artery disease and myocardial infarction, as characterized by a relative risk (RR) or odds ratio (OR) of greater than one for the particular allele or haplotype. Alternatively, the markers and/or haplotypes of the invention are characteristic of decreased susceptibility (i.e., decreased risk) of coronary artery disease and myocardial infarction, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or the can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary compute-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample”, as described herein, refers to a sample obtained from an individuals that contains nucleic acid. In certain embodiments, i.e. the detection of specific polymorphic markers and/or haplotypes, the nucleic acid sample comprises genomic DNA. Such a nucleic acid sample can be obtained from any source that contains genomic DNA, including as a blood sample, sample of amniotic fluid, sample of cerebrospinal fluid, or tissue sample from skin, muscle, buccal or conjunctival mucosa, placenta, gastrointestinal tract or other organs.

The term “CAD and/or MI therapeutic agent”, as described herein, refers to an agent that can be used to ameliorate or prevent symptoms associated with coronary artery disease or myocardial infarction. Such agents can for example be statins, beta blockers, calcium channel blockers, cardiac glycosides, antihypertensive agents, diuretics, agents acting on the renin-angiotensin system, and aspirin, or other therapeutic agents as described herein.

The term “CAD and/or MI-associated nucleic acid”, as described herein, refers to a nucleic acid that has been found to be associated to coronary artery disease and myocardial infarction. This includes, but is not limited to, the markers and haplotypes described herein and markers and haplotypes in linkage disequilibrium (LD) therewith.

The term “early onset”, as described herein, refers to onset of a disease that is lower than is typically observed. Fore example, in certain embodiments as applied to the MI phenotype, “early onset” can be defined as a MI event before the age of 50 for males and before the age of 60 for females. However, the term can be defined using other age cutoffs, as is deemed appropriate by the skilled person, and should in the present context be taken to mean age of onset of the disease that is lower than one would typically observe, and consequently considered early.

### Association of Genetic Variants to Coronary Artery Disease and Myocardial Infarction

Through a genome-wide association search for variants contributing to increased risk of developing coronary artery disease (CAD) and myocardial infarction (MI), a number of SNP variants have been found to be associated with MI and CAD. These variants, (see Table 1), and variants in linkage disequilibrium with the identified variants, can be used to assess risk of developing CAD or MI in an individual, by determining the genotype of the individual for the individual variants. Haplotypes comprising the variants of the present invention, or haplotypes in LD with the variants of the invention, can also be used to assess risk of developing CAD or MI.

The genome-wide scan was expanded to a larger discovery cohort and the analysis repeated (Table 3). Most of the SNPs showing association in the discovery sample were investigated in six replication cohorts, from Durham (NC), Atlanta (GA), Philadelphia (PA), and Johns Hopkins (MD) in the United States, from Italy and from the Wellcome Trust Case Control Consortium (WTCCC). As shown in Table 4, several variants show evidence for association in the replication samples, in particular markers rs11751605, rs6076623, rs1412444, rs2163612 and rs1029396. These markers are therefore particularly useful in the methods, uses, apparatus and kits described further herein. The association of marker rs11751605 is particularly significant; the overall P-value of association for this marker to CAD is 1.16×10⁻⁸, which is genome-wide significant after correcting for the number of tests performed (Bonferroni correction gives 0.05/300,000˜1.7×10⁻⁷).

Markers in linkage disequilibrium with any one of rs11751605, rs6076623, rs1412444, rs2163612 and rs1029396 are also useful in the methods, uses, kits and apparatus of the invention. Markers in LD with rs11751605 are particularly useful. Surrogate markers for rs11751605, based on r² values in the Hapmap Caucasian CEU sample of greater than 0.2).

The marker rs11751605 is located on Chromosome 6, near the 5′ end of the Lipoprotein(a) gene (also called Lp(a)). The Lp(a) gene encodes a lipoprotein in a subclass consisting of an LDL-like particle and the specific apolipoprotein(a) [apo(a)], which is covalently bound to the apoB of the LDL-like particle.

Apo(a) proteins vary in size due to a size polymorphism [KIV-2 VNTR], which is caused by a variable number of so called kringle IV repeats in the LPA gene. These variable apo(a) sizes are known as “apo(a) isoforms”. There is a general inverse correlation between the size of the apo(a) isoform and the Lp(a) plasma concentration

Lp(a) concentrations are highly heritable and vary over one thousandfold between individuals, from <0.2 to >200 mg/dL. Lp(a) plasma concentrations is mainly controlled by the apolipoprotein(a) gene (LPA) located on chromosome 6q26-27. However, the apo(a) size polymorphism does not explain all the interindividual variation in Lp(a) levels in plasma. For alleles with identical number of K IV repeats, the Lp(a) level can vary by a factor of 200. Additional polymorphisms (independent from the K IV size polymorphism) in the gene have been shown to affect the Lp(a) concentration (Human Molecular Genetics, 2001, 10: 815-824). Therefore, the genetic determination of Lp(a) levels is highly complex and currently not fully understood.

Lipoprotein structure is similar to plasminogen and tPA (tissue plasminogen activator) and it has been shown to affect (reduce) fibrinolysis and (increase) thrombogenesis. In addition, the LDL cholesterol content of Lp-a contributes to atherosclerosis.

High Lp(a) in blood has been shown to be a risk factor for coronary heart disease (CHD), cerebrovascular disease (CVD), atherosclerosis, thrombosis, and stroke. However, few reported studies have investigated the association between genetic variants that affect Lp(a) concentration and coronary artery disease. The frequency of small apo(a) isoforms has been shown to be higher in patients with myocardial infarction compared with healthy controls (Atheroslerosis 144:323-333 (1999)) and another study has shown the isoforms to be higher in those with advanced atherosclerosis of the carotid arteries compared to those without advanced atherosclerosis (Circulation 100:1154-1160 (1999)).

A recent study reported that a single nucleotide polymorphism (rs3798220) in the coding sequence of the LPA gene associated with severe coronary artery disease and Lp(a) levels (Arterioscler Thromb Vasc Biol 27:2030-2036 (2007)). The rs11751605 marker is however not correlated with rs3798220. In a sample of over 5000 Icelanders genotyped for both markers, the at-risk allele of rs3798220 does not lie on the haplotype background of the risk allele rs11751605 (r²=0.002). In other words, the at-risk allele of rs3798220 and the at-risk allele of rs11751605 reside on different chromosomal backgrounds, and they are thus not inherited together. As a consequence, these signals are unrelated. The allelic frequency of rs11751605 allele C (the at-risk allele) is 11%, the frequency of rs3798220 allele C (the reported at-risk allele) is 1.5%, whereas their joint frequency is zero.

Thus, the association observed for rs11751605 is clearly not due to, or correlated to, rs3798220. Moreover, the rs3798220 C allele found to associate with Lp(a) levels is very rare (1.5%), whereas the at-risk C allele of rs11751605 is relatively common (11%), thus explaining a higher proportion of the risk conferred by the variant for MI and CAD susceptibility. It is at present not known if the rs11751605 marker correlates with Lp(a) levels in plasma.

It is important to note that correlation of a certain disease (MI or CAD, for example) to certain biological risk factors (biomarkers or related traits) does not, in general translate into association of the disease to markers in the gene encoding the biomarker. Moreover, genetic risk for a confounding trait or disease, or a known risk factor for a disease, does not necessarily translate into shared genetic effects. Many such examples are known from diseases such as type 2 diabetes, LDL cholesterol and obesity, which all have observable effects on the risk of CAD. For example, the strongest genetic risk factor for type II diabetes (in the TCF7L2 gene) does not show any association with MI. The markers rs693 and rs562338, located within the ApoB locus, have been shown to strongly correlate with LDL cholesterol levels (Nature Genet. 40:161-9 (2008)), yet these markers do not show any association with CAD. In addition, genetic markers within the FTO gene that have been shown to associate with obesity are not associated with CAD.

Thus, even in light of the prior knowledge of the correlation of Lp(a) levels with risk of CAD and MI, and the identification of genetic variants within the Lp(a) gene with Lp(a) levels, it would by no means have been obvious that genetic variants within the gene confer risk of MI or CAD. If this were the case, such a genetic relationship would presumably have been established much earlier. The present finding of an association between rs11751605 and risk of CAD and MI is thus very surprising.

### Assessment for Markers and Haplotypes

The genomic sequence within populations is not identical when individuals are compared. Rather, the genome exhibits sequence variability between individuals at many locations in the genome. Such variations in sequence are commonly referred to as polymorphisms, and there are many such sites within each genome. For example, the human genome exhibits sequence variations which occur on average every 500 base pairs. The most common sequence variant consists of base variations at a single base position in the genome, and such sequence variants, or polymorphisms, are commonly called Single Nucleotide Polymorphisms (“SNPs”). These SNPs are believed to have occurred in a single mutational event, and therefore there are usually two possible alleles possible at each SNPsite; the original allele and the mutated allele. Due to natural genetic drift and possibly also selective pressure, the original mutation has resulted in a polymorphism characterized by a particular frequency of its alleles in any given population. Many other types of sequence variants are found in the human genome, including microsatellites, insertions, deletions, inversions and copy number variations. A polymorphic microsatellite has multiple small repeats of bases (such as CA repeats, TG on the complimentary strand) at a particular site in which the number of repeat lengths varies in the general population. In general terms, each version of the sequence with respect to the polymorphic site represents a specific allele of the polymorphic site. These sequence variants can all be referred to as polymorphisms, occurring at specific polymorphic sites characteristic of the sequence variant in question. In general terms, polymorphisms can comprise any number of specific alleles. Thus in one embodiment of the invention, the polymorphism is characterized by the presence of two or more alleles in any given population. In another embodiment, the polymorphism is characterized by the presence of three or more alleles. In other embodiments, the polymorphism is characterized by four or more alleles, five or more alleles, six or more alleles, seven or more alleles, nine or more alleles, or ten or more alleles. All such polymorphisms can be utilized in the methods and kits of the present invention, and are thus within the scope of the invention.

In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a trait or disease phenotype). Alleles for SNP markers as referred to herein refer to the bases A, C, G or T as they occur at the polymorphic site in the SNP assay employed. The allele codes for SNPs used herein are as follows: 1=A, 2=C, 3=G, 4=T. The person skilled in the art will however realise that by assaying or reading the opposite DNA strand, the complementary allele can in each case be measured. Thus, for a polymorphic site (polymorphic marker) containing an A/G polymorphism, the assay employed may either measure the percentage or ratio of the two bases possible, i.e. A and G. Alternatively, by designing an assay that determines the opposite strand on the DNA template, the percentage or ratio of the complementary bases T/C can be measured. Quantitatively (for example, in terms of relative risk), identical results would be obtained from measurement of either DNA strand (+strand or −strand).

Typically, a reference sequence is referred to for a particular sequence. Alleles that differ from the reference are referred to as “variant” alleles. A variant sequence, as used herein, refers to a sequence that differs from the reference sequence but is otherwise substantially similar. Alleles at the polymorphic genetic markers that make up the haplotypes described herein are variants. Additional variants can include changes that affect a polypeptide. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence, as described in detail herein. Such sequence changes alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with coronary artery disease and myocardial infarction or a susceptibility to coronary artery disease and myocardial infarction can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype refers to a segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (e.g., Chen, X. et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific methodologies available for SNP genotyping include, but are not limited to, TaqMan genotyping assays and SNPlex platforms (Applied Biosystems), mass spectrometry (e.g., MassARRAY system from Sequenom), minisequencing methods, real-time PCR, Bio-Plex system (BioRad), CEQ and SNPstream systems (Beckman), Molecular Inversion Probe array technology (e.g., Affymetrix GeneChip), and BeadArray Technologies (e.g., Illumina GoldenGate and Infinium assays). By these or other methods available to the person skilled in the art, one or more alleles at polymorphic markers, including microsatellites, SNPs or other types of polymorphic markers, can be identified.

In certain methods described herein, an individual who is at an increased susceptibility (i.e., at risk) for Coronary Artery Disease and myocardial infarction is an individual in whom at least one specific allele at one or more polymorphic marker or haplotype conferring increased susceptibility for Coronary Artery Disease and myocardial infarction is identified (i.e., at-risk marker alleles or haplotypes). In one aspect, the at-risk marker or haplotype is one that confers a significant increased risk (or susceptibility) of coronary artery disease and myocardial infarction. In one embodiment, significance associated with a marker or haplotype is measured by a relative risk (RR). In another embodiment, significance associated with a marker or haplotype is measured by an odds ratio (OR). In a further embodiment, the significance is measured by a percentage. In one embodiment, a significant increased risk is measured as a risk (relative risk and/or odds ratio) of at least 1.2, including but not limited to: at least 1.2, at least 1.3, at least 1.4, at least 1.5, at least 1.6, at least 1.7, 1.8, at least 1.9, at least 2.0, at least 2.5, at least 3.0, at least 4.0, and at least 5.0. In a particular embodiment, a risk (relative risk and/or odds ratio) of at least 1.2 is significant. In another particular embodiment, a risk of at least 1.3 is significant. In yet another embodiment, a risk of at least 1.4 is significant. In a further embodiment, a relative risk of at least 1.5 is significant. In another further embodiment, a significant increase in risk is at least 1.7 is significant. However, other cutoffs are also contemplated, e.g., at least 1.15, 1.25, 1.35, and so on, and such cutoffs are also within scope of the present invention. In other embodiments, a significant increase in risk is at least about 20%, including but not limited to about 25%, 30%, 35%, 40%, 45%, 50%, 55%, 60%, 65%, 70%, 75%, 80%, 85%, 90%, 95%, 100%, 150%, 200%, 300%, and 500%. In one particular embodiment, a significant increase in risk is at least 20%. In other embodiments, a significant increase in risk is at least 30%, at least 40%, at least 50%, at least 60%, at least 70%, at least 80%, at least 90% and at least 100%. Other cutoffs or ranges as deemed suitable by the person skilled in the art to characterize the invention are however also contemplated, and those are also within scope of the present invention. In certain embodiments, a significant increase in risk is characterized by a p-value, such as a p-value of less than 0.05, less than 0.01, less than 0.001, less than 0.0001, less than 0.00001, less than 0.000001, less than 0.0000001, less than 0.00000001, or less than 0.000000001.

An at-risk polymorphic marker or haplotype of the present invention is one where at least one allele of at least one marker or haplotype is more frequently present in an individual at risk for the disease or trait (affected), compared to the frequency of its presence in a comparison group (control), and wherein the presence of the marker or haplotype is indicative of susceptibility to coronary artery disease and/or myocardial infarction. The control group may in one embodiment be a population sample, i.e. a random sample from the general population. In another embodiment, the control group is represented by a group of individuals who are disease-free. Such disease-free control may in one embodiment be characterized by the absence of one or more specific disease-associated symptoms. In another embodiment, the disease-free control group is characterized by the absence of one or more disease-specific risk factors. Such risk factors are in one embodiment at least one environmental risk factor. Representative environmental factors are natural products, minerals or other chemicals which are known to affect, or contemplated to affect, the risk of developing the specific disease or trait. Other environmental risk factors are risk factors related to lifestyle, including but not limited to food and drink habits, geographical location of main habitat, and occupational risk factors. In another embodiment, the risk factors comprise at least one additional genetic risk factor.

As an example of a simple test for correlation would be a Fisher-exact test on a two by two table. Given a cohort of chromosomes, the two by two table is constructed out of the number of chromosomes that include both of the markers or haplotypes, one of the markers or haplotypes but not the other and neither of the markers or haplotypes. Other statistical tests of association known to the skilled person are also contemplated and are also within scope of the invention.

In other embodiments of the invention, an individual who is at a decreased susceptibility (i.e., at a decreased risk) for coronary artery disease and/or myocardial infarction is an individual in whom at least one specific allele at one or more polymorphic marker or haplotype conferring decreased susceptibility for coronary artery disease and myocardial infarction is identified. The marker alleles and/or haplotypes conferring decreased risk are also said to be protective. In one aspect, the protective marker or haplotype is one that confers a significant decreased risk (or susceptibility) of coronary artery disease and myocardial infarction. In one embodiment, significant decreased risk is measured as a relative risk (or odds ratio) of less than 0.9, including but not limited to less than 0.9, less than 0.8, less than 0.7, less than 0.6, less than 0.5, less than 0.4, less than 0.3, less than 0.2 and less than 0.1. In one particular embodiment, significant decreased risk is less than 0.7. In another embodiment, significant decreased risk is less than 0.5. In yet another embodiment, significant decreased risk is less than 0.3. In another embodiment, the decrease in risk (or susceptibility) is at least 20%, including but not limited to at least 25%, at least 30%, at least 35%, at least 40%, at least 45%, at least 50%, at least 55%, at least 60%, at least 65%, at least 70%, at least 75%, at least 80%, at least 85%, at least 90%, at least 95% and at least 98%. In one particular embodiment, a significant decrease in risk is at least about 30%. In another embodiment, a significant decrease in risk is at least about 50%. In another embodiment, the decrease in risk is at least about 70%. Other cutoffs or ranges as deemed suitable by the person skilled in the art to characterize the invention are however also contemplated, and those are also within scope of the present invention.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

A genetic variant associated with a disease or a trait (e.g. CAD or MI) can be used alone to predict the risk of the disease for a given genotype. For a biallelic marker, such as a SNP, there are 3 possible genotypes: homozygote for the at risk variant, heterozygote, and non carrier of the at risk variant. Risk associated with variants at multiple loci can be used to estimate overall risk. For multiple SNP variants, there are k possible genotypes k=3ⁿ×2^{p}; where n is the number autosomal loci and p the number of gonosomal (sex chromosomal) loci. Overall risk assessment calculations usually assume that the relative risks of different genetic variants multiply, i.e. the overall risk (e.g., RR or OR) associated with a particular genotype combination is the product of the risk values for the genotype at each locus. If the risk presented is the relative risk for a person, or a specific genotype for a person, compared to a reference population with matched gender and ethnicity, then the combined risk—is the product of the locus specific risk values—and which also corresponds to an overall risk estimate compared with the population. If the risk for a person is based on a comparison to non-carriers of the at risk allele, then the combined risk corresponds to an estimate that compares the person with a given combination of genotypes at all loci to a group of individuals who do not carry risk variants at any of those loci. The group of non-carriers of any at risk variant has the lowest estimated risk and has a combined risk, compared with itself (i.e., non-carriers) of 1.0, but has an overall risk, compare with the population, of less than 1.0. It should be noted that the group of non-carriers can potentially be very small, especially for large number of loci, and in that case, its relevance is correspondingly small.

The multiplicative model is a parsimonious model that usually fits the data of complex traits reasonably well. Deviations from multiplicity have been rarely described in the context of common variants for common diseases, and if reported are usually only suggestive since very large sample sizes are usually required to be able to demonstrate statistical interactions between loci.

By way of an example, let us consider a total of eight variants that have been described to associate with prostate cancer (Gudmundsson, J., et al., Nat Genet 39:631-7 (2007), Gudmundsson, J., et al., Nat Genet 39:977-83 (2007); Yeager, M., et al, Nat Genet 39:645-49 (2007), Amundadottir, L., et al., Nat Genet 38:652-8 (2006); Haiman, C. A., et al., Nat Genet 39:638-44 (2007)). Seven of these loci are on autosomes, and the remaining locus is on chromosome X. The total number of theoretical genotypic combinations is then 3⁷×2¹=4374. Some of those genotypic classes are very rare, but are still possible, and should be considered for overall risk assessment. It is likely that the multiplicative model applied in the case of multiple genetic variant will also be valid in conjugation with non-genetic risk variants assuming that the genetic variant does not clearly correlate with the “environmental” factor. In other words, genetic and non-genetic at-risk variants can be assessed under the multiplicative model to estimate combined risk, assuming that the non-genetic and genetic risk factors do not interact.

Using the same quantitative approach, the combined or overall risk associated with a plurality of variants associated with CAD and MI may be assessed. In certain such embodiments, the markers described herein are assessed together with at least one marker in linkage disequilibrium with the CDKN2A and/or CDKN2A genes, such as rs10811650, rs10116277, rs1333040, rs10738607, rs4977574, rs6475608, D9S1870, rs2383207, rs1333045, rs1333046, rs10757278 or rs1333048. In a preferred embodiment, the markers described herein are assessed in combination with rs10757278.

### Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randomly in the genome; rather, there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A. J., et al., Nature Genet 29:217-222 (2001); May, C. A., et al., Nature Genet 31:272-275 (2002)).

Linkage Disequilibrium (LD) refers to a non-random assortment of two genetic elements. For example, if a particular genetic element (e.g., “alleles” of a polymorphic marker) occurs in a population at a frequency of 0.50 (50%) and another occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium since they tend to be inherited together at a higher rate than what their independent allele frequencies would predict. Roughly speaking, LD is generally correlated with the frequency of recombination events between the two elements. Allele frequencies can be determined in a population by genotyping individuals in a population and determining the occurrence of each allele in the population. For populations of diploids, e.g., human populations, individuals will typically have two alleles for each genetic element (e.g., a marker or gene).

Many different measures have been proposed for assessing the strength of linkage disequilibrium (LD). Most capture the strength of association between pairs of biallelic sites. Two important pairwise measures of LD are r² (sometimes denoted Δ²) and |D′|. Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. So, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, or at least 0.99. In one preferred embodiment, the significant r² value can be at least 0.2. Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98, or at least 0.99. Thus, linkage disequilibrium represents a correlation between alleles of distinct markers. It is measured by correlation coefficient or |D′| (r² up to 1.0 and |D′| up to 1.0). In certain embodiments, linkage disequilibrium is defined in terms of values for both the r² and |D′| measures. In one such embodiment, a significant linkage disequilibrium is defined as r²>0.1 and |D′|>0.8. In another embodiment, a significant linkage disequilibrium is defined as r²>0.2 and |D′|>0.9. Other combinations and permutations of values of r² and |D′| for determining linkage disequilibrium are also contemplated, and are also within the scope of the invention. Linkage disequilibrium can be determined in a single human population, as defined herein, or it can be determined in a collection of samples comprising individuals from more than one human population. In one embodiment of the invention, LD is determined in a sample from one or more of the HapMap populations (caucasian, african, japanese, chinese), as defined (http://www.hapmap.org). In one such embodiment, LD is determined in the Caucasian CEU population of the HapMap samples. In another embodiment, LD is determined in the African YRI population. In yet another embodiment, LD is determined in samples from the Icelandic population.

If all polymorphisms in the genome were independent at the population level (i.e., no LD), then every single one of them would need to be investigated in association studies, to assess all the different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the present invention. One or more causative (functional) variants or mutations may reside within the region found to be associating to the disease or trait. Such variants may confer a higher relative risk (RR) or odds ratio (OR) than observed for the tagging markers used to detect the association. The present invention thus refers to the markers used for detecting association to the disease, as described herein, as well as markers in linkage disequilibrium with the markers. Thus, in certain embodiments of the invention, markers that are in LD with the markers and/or haplotypes of the invention, as described herein, may be used as surrogate markers. The surrogate markers have in one embodiment relative risk (RR) and/or odds ratio (OR) values smaller than for the markers or haplotypes initially found to be associating with the disease, as described herein. In other embodiments, the surrogate markers have RR or OR values greater than those initially determined for the markers initially found to be associating with the disease, as described herein. An example of such an embodiment would be a rare, or relatively rare (such as <10% allelic population frequency) variant in LD with a more common variant (>10% population frequency) initially found to be associating with the disease, such as the variants described herein. Identifying and using such markers for detecting the association discovered by the inventors as described herein can be performed by routine methods well known to the person skilled in the art, and are therefore within the scope of the present invention.

### Determination of Haplotype Frequency

The frequencies of haplotypes in patient and control groups can be estimated using an expectation-maximization algorithm (Dempster A. et al., J. R. Stat. Soc. B, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk and protective markers and haplotypes within a linkage region, for example, association of all possible combinations of genotyped markers is studied, provided those markers span a practical region. The combined patient and control groups can be randomly divided into two sets, equal in size to the original group of patients and controls. The marker and haplotype analysis is then repeated and the most significant p-value registered is determined. This randomization scheme can be repeated, for example, over 100 times to construct an empirical distribution of p-values. In a preferred embodiment, a p-value of <0.05 is indicative of an significant marker and/or haplotype association.

### Haplotype Analysis

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

For single marker association to a disease, the Fisher exact test can be used to calculate two-sided p-values for each individual allele. Usually, all p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families for the linkage analysis, first and second-degree relatives can be eliminated from the patient list. Furthermore, the test can be repeated for association correcting for any remaining relatedness among the patients, by extending a variance adjustment procedure described in Risch, N. & Teng, J. (Genome Res., 8:1273-1288 (1998)), DNA pooling (ibid) for sibships so that it can be applied to general familial relationships, and present both adjusted and unadjusted p-values for comparison. The differences are in general very small as expected. To assess the significance of single-marker association corrected for multiple testing we can carry out a randomization test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts. Alternatively, a test of association is done in one or several additional case-control cohorts in replication analyses. For tests of single markers, a replication sample analysis only needs to fulfill nominal statistical significance of P<0.05 (two-sided testing; P<0.1 may in fact be more appropriate since a single predefined effect of a single marker is being tested). If many markers are tested in a replication effort, a correction for the number of markers tested in the replication analysis is performed. Alternatively, the results of replication in several case-control cohorts can be combined to provide an overall assessment of the underlying effect, and this may be the most appropriate way of testing the validity of the initially discovered association signal. Deficiencies in individual case-control comparisons, for example due to recruitment bias or population can be neutralized by combined analysis of several case-control studies. The most common method for performing such analysis is the Mantel Haenszel model, which allows different population frequencies for alleles, haplotypes and genotypes but assumes a common relative risk (Mantel, N. & Haenszel, W., J Natl Cancer Inst 22:719 (1959)).

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affected and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

### Linkage Disequilibrium Using NEMO

LD between pairs of markers can be calculated using the standard definition of D′ and r² (Lewontin, R., Genetics 49:49-67 (1964); Hill, W. G. & Robertson, A. Theor. Appl. Genet. 22:226-231 (1968)). Using NEMO, frequencies of the two marker allele combinations are estimated by maximum likelihood and deviation from linkage equilibrium is evaluated by a likelihood ratio test. The definitions of D′ and r² are extended to include microsatellites by averaging over the values for all possible allele combination of the two markers weighted by the marginal allele probabilities. When plotting all marker combination to elucidate the LD structure in a particular region, we plot D′ in the upper left corner and the p-value in the lower right corner. In the LD plots the markers can be plotted equidistant rather than according to their physical location, if desired.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The Risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

In certain embodiments, marker and haplotype analysis involves defining a candidate susceptibility region or locus based on “haplotype blocks” (also called “LD blocks”). It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). As used herein, the terms “haplotype block” or “LD block” includes blocks defined by either characteristic.

Representative methods for identification of haplotype blocks are set forth, for example, in U.S. Published Patent Application Nos. 20030099964, 20030170665, 20040023237 and 20040146870. Haplotype blocks can be used readily to map associations between phenotype and haplotype status. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

As described herein, certain markers and haplotypes comprising such markers are found to be useful for risk assessment of cardiovascular disease, including coronary artery disease and myocardial infarction. Risk assessment can involve the use of the markers and variants for diagnosing a susceptibility to cardiovascular disease, including coronary artery disease and myocardial infarction. Particular alleles of polymorphic markers and haplotypes are found more frequently in individuals with cardiovascular disease, including coronary artery disease and myocardial infarction than in individuals without diagnosis of cardiovascular disease, including coronary artery disease and myocardial infarction. Therefore, these marker alleles and haplotypes have predictive value for detecting cardiovascular disease, including coronary artery disease and myocardial infarction, or a susceptibility to coronary artery disease and myocardial infarction, in an individual. Tagging markers within haplotype blocks or LD blocks comprising at-risk markers, such as the markers of the present invention, can be used as surrogates for other markers within the haplotype block or LD block. Markers with values of r² equal to 1 are perfect surrogates for the at-risk variants, i.e. genotypes for one marker perfectly predicts genotypes for the other. Markers with smaller values of r² than 1 can also be surrogates for the at-risk variant, or alternatively represent variants with relative risk values as high or possibly even higher than the at-risk variant. The at-risk variant identified may therefore not be the functional variant itself, but is in this instance in linkage disequilibrium with the true functional variant. The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation genotype surrogate markers in linkage disequilibrium with the markers and/or haplotypes as described herein. The tagging or surrogate markers in LD with the at-risk variants detected also have predictive value for detecting association to CAD and/or MI, or a susceptibility to CAD and/or MI, in an individual. These tagging or surrogate markers that are in LD with the markers of the present invention can also include other markers that distinguish among haplotypes in associated genomic regions, as these similarly have predictive value for detecting susceptibility to CAD and MI.

The present invention can in certain embodiments be practiced by assessing a sample comprising genomic DNA from an individual for the presence of variants described herein to be associated with the cardiovascular diseases CAD and MI. Such assessment includes steps of detecting the presence or absence of at least one allele of at least one polymorphic marker, using methods well known to the skilled person and further described herein, and based on the outcome of such assessment, determine whether the individual from whom the sample is derived is at increased or decreased risk (increased or decreased susceptibility) of CAD and/or MI. Alternatively, the invention can be practiced utilizing a dataset comprising information about the genotype status of at least one polymorphic marker described herein to be associated with CAD and/or MI (or markers in linkage disequilibrium with at least one marker shown herein to be associated with CAD and/or MI). In other words, a dataset containing information about such genetic status, for example in the form of genotype counts at a certain polymorphic marker, or a plurality of markers (e.g., an indication of the presence or absence of certain at-risk alleles), or actual genotypes for one or more markers, can be queried for the presence or absence of certain at-risk alleles at certain polymorphic markers shown by the present inventors to be associated with CAD and/or MI. A positive result for a variant (e.g., marker allele) associated with CAD and/or MI, as shown herein, is indicative of the individual from which the dataset is derived is at increased susceptibility (increased risk) of CAD and/or MI.

In certain embodiments of the invention, a polymorphic marker is correlated to CAD and/or MI by referencing genotype data for the polymorphic marker to a look-up table that comprises correlations between at least one allele of the polymorphism and CAD and/or MI. In some embodiments, the table comprises a correlation for one polymorphism. In other embodiments, the table comprises a correlation for a plurality of polymorphisms. In both scenarios, by referencing to a look-up table that gives an indication of a correlation between a marker and CAD and/or MI, a risk for CAD and/or MI, or a susceptibility to CAD and/or MI, can be identified in the individual from whom the sample is derived. In some embodiments, the correlation is reported as a statistical measure. The statistical measure may be reported as a risk measure, such as a relative risk (RR), an absolute risk (AR) or an odds ratio (OR).

The markers and haplotypes of the invention, e.g., the markers presented in Table 4, may be useful for risk assessment and diagnostic purposes for coronary artery disease and myocardial infarction, either alone or in combination. Thus, even in the cases where the increase in risk by individual markers is relatively modest, i.e. on the order of 10-30%, the association may have significant implications. Thus, relatively common variants may have significant contribution to the overall risk (Population Attributable Risk is high), or combination of markers can be used to define groups of individual who, based on the combined risk of the markers, is at significant combined risk of developing CAD and/or MI. Thus, in one embodiment of the invention, a plurality of variants (markers and/or haplotypes) is used for overall risk assessment. In such embodiments, the genotype status of a plurality of markers and/or haplotypes is determined in an individual, and the status of the individual compared with the population frequency of the associated variants, or the frequency of the variants in clinically healthy subjects, such as age-matched and sex-matched subjects. Methods known in the art, such as multivariate analyses or joint risk analyses, may subsequently be used to determine the overall risk conferred based on the genotype status at the multiple loci. Assessment of risk based on such analysis may subsequently be used in the methods and kits of the invention, as described herein.

As described in the above, the haplotype block structure of the human genome has the effect that a large number of variants (markers and/or haplotypes) in linkage disequilibrium with the variant originally associated with a disease or trait may be used as surrogate markers for assessing association to the disease or trait. The number of such surrogate markers will depend on factors such as the historical recombination rate in the region, the mutational frequency in the region (i.e., the number of polymorphic sites or markers in the region), and the extent of LD (size of the LD block) in the region. These markers are usually located within the physical boundaries of the LD block or haplotype block in question as defined using the methods described herein, or by other methods known to the person skilled in the art. However, sometimes marker and haplotype association is found to extend beyond the physical boundaries of the haplotype block as defined. Such markers and/or haplotypes may in those cases be also used as surrogate markers and/or haplotypes for the markers and/or haplotypes physically residing within the haplotype block as defined. As a consequence, markers and haplotypes in LD (typically characterized by r² greater than 0.1, such as r² greater than 0.2, including r² greater than 0.3, also including r² greater than 0.4) with the markers and haplotypes of the present invention are also within the scope of the invention, even if they are physically located beyond the boundaries of the haplotype block as defined. This includes markers that are described herein (e.g., Table 4), but may also include other markers that are in strong LD (characterized by r² greater than 0.1 or 0.2 and/or |D′|>0.8) with one or more of the markers listed in Table 4.

For the SNP markers described herein, the opposite allele to the allele found to be in excess in patients (at-risk allele) is found in decreased frequency in coronary artery disease and myocardial infarction. These markers and haplotypes in LD and/or comprising such markers, are thus protective for coronary artery disease and myocardial infarction, i.e. they confer a decreased risk or susceptibility of individuals carrying these markers and/or haplotypes developing coronary artery disease and myocardial infarction.

Certain variants of the present invention, including certain haplotypes comprise, in some cases, a combination of various genetic markers, e.g., SNPs and microsatellites. Detecting haplotypes can be accomplished by methods known in the art and/or described herein for detecting sequences at polymorphic sites. Furthermore, correlation between certain haplotypes or sets of markers and disease phenotype can be verified using standard techniques. A representative example of a simple test for correlation would be a Fisher-exact test on a two by two table.

In specific embodiments, a marker allele or haplotype associated with coronary artery disease and myocardial infarction (e.g., marker alleles as listed in Table 4) is one in which the marker allele or haplotype is more frequently present in an individual at risk for coronary artery disease and/or myocardial infarction (affected), compared to the frequency of its presence in a healthy individual (control), wherein the presence of the marker allele or haplotype is indicative of coronary artery disease and/or myocardial infarction or a susceptibility to coronary artery disease and/or myocardial infarction. In other embodiments, at-risk markers in linkage disequilibrium with one or more markers found to be associated with coronary artery disease and/or myocardial infarction (e.g., marker alleles as listed in Table 4) are tagging markers that are more frequently present in an individual at risk for coronary artery disease and/or myocardial infarction (affected), compared to the frequency of their presence in a healthy individuals (controls), wherein the presence of the tagging markers is indicative of increased susceptibility to coronary artery disease and/or myocardial infarction. In a further embodiment, at-risk markers alleles (i.e. conferring increased susceptibility) in linkage disequilibrium with one or more markers found to be associated with coronary artery disease and/or myocardial infarction (e.g., marker alleles as listed in Table 4), are markers comprising one or more allele that is more frequently present in an individual at risk for coronary artery disease and/or myocardial infarction, compared to the frequency of their presence in a healthy individual (control), wherein the presence of the markers is indicative of increased susceptibility to coronary artery disease and/or myocardial infarction.

### Utility of Genetic Testing

The person skilled in the art will appreciate and understand that the variants described herein in general do not, by themselves, provide an absolute identification of individuals who will develop coronary artery disease or myocardial infarction. The variants described herein do however indicate increased and/or decreased likelihood that individuals carrying the at-risk or protective variants of the invention will develop CAD or MI, or develop symptoms associated with these diseases. This information is however extremely valuable in itself, as outlined in more detail in the below, as it can be used to, for example, initiate preventive measures at an early stage, perform regular physical and/or mental exams to monitor the progress and/or appearance of symptoms, or to schedule exams at a regular interval to identify early symptoms, so as to be able to apply treatment at an early stage.

The knowledge about a genetic variant that confers a risk of developing cardiovascular disease, including coronary artery disease and myocardial infarction, offers the opportunity to apply a genetic test to distinguish between individuals with increased risk of developing the disease (i.e. carriers of the at-risk variant) and those with decreased risk of developing the disease (i.e. carriers of the protective variant). The core values of genetic testing, for individuals belonging to both of the above mentioned groups, are the possibilities of being able to diagnose disease, or a predisposition to disease, at an early stage and provide information to the clinician about prognosis/aggressiveness of disease in order to be able to apply the most appropriate treatment.

Individuals with a family history of CAD and/or MI and carriers of at-risk variants may benefit from genetic testing since the knowledge of the presence of a genetic risk factor, or evidence for increased risk of being a carrier of one or more risk factors, may provide increased incentive for implementing a healthier lifestyle (e.g., lose weight, increase exercise, give um smoking, reduce stress, etc.), by avoiding or minimizing known environmental risk factors for these cardiovascular diseases. Genetic testing of patients may furthermore give valuable information about the primary cause of the disease and can aid the clinician in selecting the best treatment options and medication for each individual.

The present invention can be thus be used for risk assessment for coronary artery disease and/or myocardial infarction, including diagnosing whether an individual is at risk for developing myocardial infarction and/or coronary artery disease. The polymorphic markers of the present invention can be used alone or in combination, as well as in combination with other factors, including known biomarkers, for risk assessment of an individual for coronary artery disease and/or myocardial infarction. Many factors known to affect the predisposition of individual towards developing risk of developing cardiovascular disease, including coronary artery disease and/or myocardial infarction, are known to the person skilled in the art and can be utilized in such assessment. These include, but are not limited to, age, gender, smoking status, physical activity, waist-to-hip circumference ratio, family history of Cardiovascular Disease (e.g., MI and/or CAD), previously diagnosed cardiovascular disease, obesity, diagnosis of Diabetes mellitus, stress, depression, elevated heart rate, hypertriglyceridemia, low HDL cholesterol, hypertension, elevated blood pressure, cholesterol levels, HDL cholesterol, LDL cholesterol, triglycerides, apolipoprotein AI and B levels, fibrinogen, ferritin, C-reactive protein and leukotriene levels. Methods known in the art can be used for such assessment, including multivariate analyses or logistic regression, as described further herein.

### Methods

Methods for risk management and risk assessment of CAD and MI are described herein and are encompassed by the invention. The invention also encompasses methods of assessing an individual for probability of response to a therapeutic agent for a CAD and/or MI, methods for predicting the effectiveness of a therapeutic agent for CAD and/or MI, nucleic acids, polypeptides and antibodies and computer-implemented functions useful in such methods. Kits for assaying a sample from a subject to detect susceptibility to cardiovascular disease are also encompassed by the invention.

### Diagnostic and Screening Methods

In certain embodiments, the present invention pertains to methods of diagnosing, or aiding in the diagnosis of coronary artery disease (CAD) and/or myocardial infarction (MI) or a susceptibility to CAD and/or MI, by detecting particular alleles at genetic markers that appear more frequently in individuals diagnosed with CAD and MI or individuals who are susceptible to CAD and MI. In a particular embodiment, the invention is a method of diagnosing a susceptibility to CAD and/or MI by detecting at least one allele of at least one polymorphic marker (e.g., the markers described herein). The present invention describes methods whereby detection of particular alleles of particular markers or haplotypes is indicative of a susceptibility to CAD and MI. Such prognostic or predictive assays can also be used to determine prophylactic treatment of a subject prior to the onset of symptoms of cardiovascular disease, including coronary artery disease and myocardial infarction. The present invention pertains in some embodiments to methods of clinical applications of diagnosis, e.g., diagnosis performed by a medical professional. In other embodiments, the invention pertains to methods of diagnosis or determination of a susceptibility performed by a layman. The layman can be the customer of a genotyping service. The layman may also be a genotype service provider, who performs genotype analysis on a DNA sample from an individual, in order to provide service related to genetic risk factors for particular traits or diseases, based on the genotype status of the individual (i.e., the customer). A third party who provides service to individuals can be one who provides a guidance or teaching as to how to relate or interpret genotype information to calculate or estimate genetic risk. In one embodiment, the third party may receive a sample containing genomic DNA from a customer, send the sample, or DNA isolated from the sample, to a genotype service provider, and present the customer with the genotype data and/or an interpretation of the genotype data, such as by calculating disease risk for specific markers. The customer may access his/her genotype information, including results of any risk calculations, via a secure we interface. Alternatively, the customer may receive disease risk information by other means, such as by regular mail. Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, made available to the individual can be compared to information from the public literature about disease or trait risk associated with various SNPs. The diagnostic application of disease-associated alleles as described herein, can thus for example be performed by the individual, through analysis of his/her genotype data, by a health professional based on results of a clinical test, or by a third party. In other words, the diagnosis or determination of a susceptibility of genetic risk can be made by health professionals, genetic counselors, third parties providing such service or by the layman, based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to any available diagnostic method, including those mentioned above.

In certain embodiments, a sample containing genomic DNA from an individual is collected. Such sample can for example be a buccal swab, a saliva sample, a blood sample, or other suitable samples containing genomic DNA, as described further herein. The genomic DNA is then analyzed using any common technique available to the skilled person, such as high-throughput array technologies. Results from such genotyping are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for a particular human conditions, such as the genetic variants described herein. Genotype data can be retrieved from the data storage unit using any convenient data query method. Calculating risk conferred by a particular genotype for the individual is based on comparing the genotype to previously determined risk (expressed as a relative risk (RR) or and odds ratio (OR), for example) for the genotype, for example for an heterozygous carrier of an at-risk variant for a particular disease or trait (such as CAD and/or MI). Risk presented can be the relative risk for a person, or a specific genotype of a person, compared to the population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. Using the population average may in certain embodiments be more convenient, since it provides a measure which is easy to interpret for the user, i.e. a measure that gives the risk for the individual, based on his/her genotype, compared with the average in the population.

Overall risk for multiple risk variants can be performed using standard methodology. For example, assuming a multiplicative model, i.e. assuming that the risk of individual risk variants multiply to establish the overall effect, allows for a straight-forward calculation of the overall risk for multiple markers.

In addition, in certain other embodiments, the present invention pertains to methods of diagnosing, or aiding in the diagnosis of, a decreased susceptibility to cardiovascular disease, in particular CAD and/or MI, by detecting particular genetic marker alleles or haplotypes that appear less frequently in individuals diagnosed with CAD and/or MI than in individual not diagnosed with these diseases, or in the general population.

As described and exemplified herein, particular marker alleles or haplotypes (e.g. the markers as set forth in Table 4 and markers in linkage disequilibrium therewith) are associated with risk of coronary artery disease and/or myocardial infarction. In one embodiment, the marker allele or haplotype is one that confers a significant risk or susceptibility to coronary artery disease and/or myocardial infarction. In another embodiment, the invention relates to a method of diagnosing a susceptibility to CAD and/or MI in a human individual, the method comprising determining the presence or absence of at least one allele of at least one polymorphic marker in a nucleic acid sample obtained from the individual, wherein the at least one polymorphic marker is selected from the group consisting of the polymorphic markers listed in Table 4, and markers in linkage disequilibrium therewith. In another embodiment, the invention pertains to methods of diagnosing a susceptibility to CAD and/or MI in a human individual, by screening for at least one marker allele or haplotype as listed in Table 4 or markers in linkage disequilibrium therewith. In another embodiment, the marker allele or haplotype is more frequently present in a subject having, or who is susceptible to, CAD and/or MI (affected), as compared to the frequency of its presence in a healthy subject (control, such as population controls). In certain embodiments, the significance of association of the at least one marker allele or haplotype is characterized by a p value <0.05. In other embodiments, the significance of association is characterized by smaller p-values, such as <0.01, <0.001, <0.0001, <0.00001, <0.000001, <0.0000001, <0.00000001 or <0.000000001.

In these embodiments, the presence of the at least one marker allele or haplotype is indicative of a susceptibility to coronary artery disease and/or myocardial infarction. These diagnostic methods involve detecting the presence or absence of at least one marker allele or haplotype that is associated with coronary artery disease and/or myocardial infarction. The haplotypes described herein include combinations of alleles at various genetic markers (e.g., SNPs, microsatellites). The detection of the particular genetic marker alleles that make up the particular haplotypes can be performed by a variety of methods described herein and/or known in the art. For example, genetic markers can be detected at the nucleic acid level (e.g., by direct nucleotide sequencing or by other means known to the skilled in the art) or at the amino acid level if the genetic marker affects the coding sequence of a protein encoded by a coronary artery disease and/or myocardial infarction-associated nucleic acid (e.g., by protein sequencing or by immunoassays using antibodies that recognize such a protein). The marker alleles or haplotypes of the present invention correspond to fragments of a genomic DNA sequence associated with coronary artery disease and/or myocardial infarction. Such fragments encompass the DNA sequence of the polymorphic marker or haplotype in question, but may also include DNA segments in strong LD (linkage disequilibrium) with the marker or haplotype.

In one embodiment, diagnosis of a susceptibility to CAD and/or MI can be accomplished using hybridization methods, including, but not limited to, hybridization to a microarray, Southern analysis, Northern analysis, and/or in situ hybridizations (see Current Protocols in Molecular Biology, Ausubel, F. et al., eds., John Wiley & Sons, including all supplements). The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. In one embodiment, a haplotype can be indicated by a single nucleic acid probe that is specific for the specific haplotype (i.e., hybridizes specifically to a DNA strand comprising the specific marker alleles characteristic of the haplotype). A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample. Other methods of using hybridization probes in allele-specific detection methods include methods of single base extension of nucleotide probes flanking a single base polymorphic sites. Such methods are well known to the skilled artisan.

To determine a susceptibility or diagnose a susceptibility, a hybridization sample is formed by contacting the test sample containing an nucleic acid associated with CAD and/or MI, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence of any one of SEQ ID NO:1-172, as set forth herein, or the probe can be the complementary sequence of such a sequence. Other suitable probes for use in the diagnostic assays of the invention are described herein. Hybridization can be performed by methods well known to the person skilled in the art (see, e.g., Current Protocols in Molecular Biology, Ausubel, F. et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the present invention, or markers that make up a haplotype of the present invention, or multiple probes can be used concurrently to detect more than one marker alleles at a time. It is also possible to design a single probe containing more than one marker alleles of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., a haplotype) and therefore is susceptible to CAD and/or MI.

In one preferred embodiment, a method utilizing a detection oligonucleotide probe comprising a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In another hybridization method, Northern analysis (see Current Protocols in Molecular Biology, Ausubel, F. et al., eds., John Wiley & Sons, supra) is used to identify the presence of a polymorphism associated with CAD and/or MI. For Northern analysis, a test sample of RNA is obtained from the subject by appropriate means. As described herein, specific hybridization of a nucleic acid probe to RNA from the subject is indicative of a particular allele complementary to the probe. For representative examples of use of nucleic acid probes, see, for example, U.S. Pat. Nos. 5,288,611 and 4,851,330.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the marker alleles or haplotypes that are associated with CAD and/or MI. Hybridization of the PNA probe is thus diagnostic for a susceptibility of CAD and/or MI.

In one embodiment of the invention, a test sample containing genomic DNA obtained from the subject is collected and the polymerase chain reaction (PCR) is used to amplify a fragment comprising one or more markers or haplotypes of the present invention. As described herein, identification of a particular marker allele or haplotype associated with CAD and/MI, can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis using quantitative PCR (kinetic thermal cycling). This technique can, for example, utilize commercially available technologies, such as TaqMan® (Applied Biosystems, Foster City, Calif.). The technique can assess the presence of an alteration in the expression or composition of a polypeptide or splicing variant(s) that is encoded by a nucleic acid associated with CAD and/or MI, as described herein. Further, the expression of the variant(s) can be quantified as physically or functionally different.

In another embodiment of the methods of the invention, analysis by restriction digestion can be used to detect a particular allele if the allele results in the creation or elimination of a restriction site relative to a reference sequence. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles or haplotypes associated with CAD and/or MI (e.g. the polymorphic markers of Table 4 and markers in linkage disequilibrium therewith). Therefore, in one embodiment, determination of the presence or absence of a particular marker alleles or haplotypes comprises sequence analysis of a test sample of DNA or RNA obtained from a subject or individual. PCR or other appropriate methods can be used to amplify a portion of a nucleic acid template, and the presence of a specific allele can then be detected directly by sequencing the polymorphic site (or multiple polymorphic sites in a haplotype) of the genomic DNA in the sample.

In another embodiment, arrays of oligonucleotide probes that are complementary to target nucleic acid sequence segments from a subject, can be used to identify polymorphisms in a nucleic acid associated with CAD and/or MI (e.g. the polymorphic markers of Table 4 and markers in linkage disequilibrium therewith). For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Fodor, S. et al., Science, 251:767-773 (1991); Pirrung et al., U.S. Pat. No. 5,143,854 (see also published PCT Application No. WO 90/15070); and Fodor. S. et al., published PCT Application No. WO 92/10092 and U.S. Pat. No. 5,424,186, the entire teachings of each of which are incorporated by reference herein). Techniques for the synthesis of these arrays using mechanical synthesis methods are described in, e.g., U.S. Pat. No. 5,384,261; the entire teachings of which are incorporated by reference herein. In another example, linear arrays can be utilized. Additional descriptions of use of oligonucleotide arrays for detection of polymorphisms can be found, for example, in U.S. Pat. Nos. 5,858,659 and 5,837,832, the entire teachings of both of which are incorporated by reference herein.

Other methods of nucleic acid analysis that are available to those skilled in the art can be used to detect a particular allele at a polymorphic site associated with CAD and/or MI (e.g. the polymorphic markers of Table 4 and markers in linkage disequilibrium therewith). Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, diagnosis of CAD and/or MI or a susceptibility to CAD and/or MI can be made by examining expression and/or composition of a polypeptide encoded by a nucleic acid associated with these diseases, in those instances where the genetic marker(s) or haplotype(s) of the present invention result in a change in the composition or expression of the polypeptide. Thus, diagnosis or determination of a susceptibility can be made by examining expression and/or composition of one of these polypeptides, or another polypeptide encoded by a nucleic acid associated with CAD and/or MI, in those instances where the genetic marker or haplotype of the present invention results in a change in the composition or expression of the polypeptide. The haplotypes and markers of the present invention that show association to CAD and MI may play a role through their effect on one or more of these nearby genes (e.g., the Lp(a) gene). Possible mechanisms affecting these genes include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation.

Thus, in another embodiment, the variants (markers or haplotypes) of the invention showing association to CAD and/or MI affect the expression of a nearby gene, such as the Lp(a) gene. It is well known that regulatory element affecting gene expression may be located far away, even as far as tenths or hundreds of kilobases away, from the promoter region of a gene. By assaying for the presence or absence of at least one allele of at least one polymorphic marker of the present invention, it is thus possible to assess the expression level of such nearby genes. It is thus contemplated that the detection of the markers or haplotypes of the present invention can be used for assessing expression of genes associated with (in LD with) the associated markers.

A variety of methods can be used for detecting protein expression levels, including enzyme linked immunosorbent assays (ELISA), Western blots, immunoprecipitations and immunofluorescence. A test sample from a subject is assessed for the presence of an alteration in the expression and/or an alteration in composition of the polypeptide encoded by a nucleic acid associated with CAD and/or MI. An alteration in expression of a polypeptide encoded by a nucleic acid associated with CAD and/or MI can be, for example, an alteration in the quantitative polypeptide expression (i.e., the amount of polypeptide produced). An alteration in the composition of a polypeptide encoded by a nucleic acid associated with CAD and/or MI is an alteration in the qualitative polypeptide expression (e.g., expression of a mutant polypeptide or of a different splicing variant). In one embodiment, diagnosis of a susceptibility to CAD and/or MI is made by detecting a particular splicing variant encoded by a nucleic acid associated with these diseases, or a particular pattern of splicing variants.

Both such alterations (quantitative and qualitative) can also be present. An “alteration” in the polypeptide expression or composition, as used herein, refers to an alteration in expression or composition in a test sample, as compared to the expression or composition of the polypeptide in a control sample. A control sample is a sample that corresponds to the test sample (e.g., is from the same type of cells), and is from a subject who is not affected by, and/or who does not have a susceptibility to, CAD and/or MI. In one embodiment, the control sample is from a subject that does not possess a marker allele or haplotype associated with CAD and/or MI, as described herein. Similarly, the presence of one or more different splicing variants in the test sample, or the presence of significantly different amounts of different splicing variants in the test sample, as compared with the control sample, can be indicative of a susceptibility to CAD and/or MI. An alteration in the expression or composition of the polypeptide in the test sample, as compared with the control sample, can be indicative of a specific allele in the instance where the allele alters a splice site relative to the reference in the control sample. Various means of examining expression or composition of a polypeptide encoded by a nucleic acid are known to the person skilled in the art and can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a nucleic acid associated with CAD and/or MI can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of polypeptide encoded by a nucleic acid associated with CAD and/or MI in a test sample is compared with the level or amount of the polypeptide in a control sample. A level or amount of the polypeptide in the test sample that is higher or lower than the level or amount of the polypeptide in the control sample, such that the difference is statistically significant, is indicative of an alteration in the expression of the polypeptide encoded by the nucleic acid, and is diagnostic for a particular allele or haplotype responsible for causing the difference in expression. Alternatively, the composition of the polypeptide in a test sample is compared with the composition of the polypeptide in a control sample. In another embodiment, both the level or amount and the composition of the polypeptide can be assessed in the test sample and in the control sample.

In another embodiment, the diagnosis of a susceptibility to CAD and/or MI is made by detecting at least one marker or haplotypes of the present invention (e.g., associated alleles of the markers listed in Table 4, and markers in linkage disequilibrium therewith), in combination with an additional protein-based, RNA-based or DNA-based assay.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies that bind to an altered polypeptide encoded by a nucleic acid of the invention as described herein (e.g., a genomic segment comprising at least one polymorphic marker and/or haplotype of the present invention) or to a non-altered (native) polypeptide encoded by a nucleic acid of the invention as described herein, means for amplification of a nucleic acid templates, means for analyzing the nucleic acid sequence of a nucleic acids, means for analyzing the amino acid sequence of a polypeptide encoded by a nucleic acid associated with CAD and/or MI, etc. The kits can for example include necessary buffers, nucleic acid primers for amplifying nucleic acids of the invention (e.g., a nucleic acid segment comprising one or more of the polymorphic markers as described herein), and reagents for allele-specific detection of the fragments amplified using such primers and necessary enzymes (e.g., DNA polymerase). Additionally, kits can provide reagents for assays to be used in combination with the methods of the present invention, e.g., reagents for use with other diagnostic assays for CAD and/or MI.

In one embodiment, the invention is a kit for assaying a sample from a subject to detect a susceptibility to coronary artery disease and/or myocardial infarction in a subject, wherein the kit comprises reagents necessary for selectively detecting at least one allele of at least one polymorphism of the present invention in the genome of the individual. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes one polymorphism, wherein the polymorphism is selected from the group consisting of the polymorphisms as defined in and polymorphic markers in linkage disequilibrium therewith. In yet another embodiment the fragment is at least 20 base pairs in size. Such oligonucleotides or nucleic acids (e.g., oligonucleotide primers) can be designed using portions of the nucleic acids flanking polymorphisms (e.g., SNPs or microsatellites) that are indicative of coronary artery disease and myocardial infarction. In another embodiment, the kit comprises one or more labeled nucleic acids capable of detecting one or more specific polymorphic markers or haplotypes associated with coronary artery disease and/or myocardial infarction, and reagents for detection of the label. Suitable labels include, e.g., a radioisotope, a fluorescent label, an enzyme label, an enzyme co-factor label, a magnetic label, a spin label, an epitope label.

In particular embodiments, the polymorphic marker or haplotype to be detected by the reagents of the kit comprises one or more markers, two or more markers, three or more markers, four or more markers or five or more markers selected from the group consisting of the markers in Table 3. In another embodiment, the marker or haplotype to be detected comprises the markers listed in Table 4, and markers in linkage disequilibrium therewith. In another embodiment, the marker or haplotype to be detected is selected from marker rs11751605, rs6076623, rs1412444, rs2163612, rs1029396, rs2243547, rs12534186, rs12134779 and rs7158073. In another embodiment, the marker or haplotype to be detected is selected from marker rs11751605, rs6076623, rs1412444, rs2163612 and rs1029396. In another embodiment, the marker or haplotype to be detected is marker rs11751605. In such embodiments, the presence of the marker or haplotype is indicative of a susceptibility (increased susceptibility or decreased susceptibility) to cardiovascular disease, including coronary artery disease and myocardial infarction.

In one preferred embodiment, the kit for detecting the markers of the invention comprises a detection oligonucleotide probe, that hybridizes to a segment of template DNA containing a SNP polymorphisms to be detected, an enhancer oligonucleotide probe and an endonuclease. As explained in the above, the detection oligonucleotide probe comprises a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection, and primers for such amplification are included in the reagent kit. In such an embodiment, the amplified DNA serves as the template for the detection probe and the enhancer probe.

In one embodiment, the DNA template is amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In one of such embodiments, the presence of the marker or haplotype is indicative of a susceptibility (increased susceptibility or decreased susceptibility) to CAD and/or MI. In another embodiment, the presence of the marker or haplotype is indicative of response to a therapeutic agent for a cardiovascular disease, such as CAD and MI. In another embodiment, the presence of the marker or haplotype is indicative of prognosis of MI and/or CAD. In yet another embodiment, the presence of the marker or haplotype is indicative of progress of treatment of CAD and/or MI. Such treatment may include intervention by surgery, medication or by other means (e.g., lifestyle changes).

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention is instructed to take a prescribed dose of the therapeutic agent. In another embodiment, an individual identified as a non-carrier of at least one variant of the present invention is instructed to take a prescribed dose of the therapeutic agent.

In certain embodiments, the kit further comprises a set of instructions for using the reagents comprising the kit.

### Therapeutic Agents

Variants of the present invention (e.g., the markers and/or haplotypes of the invention as described herein, e.g., the markers listed in Table 3 and 4) can be used to identify novel therapeutic targets for coronary artery disease or myocardial infarction, or other cardiovascular disease. For example, genes containing, or in linkage disequilibrium with, variants (markers and/or haplotypes) associated with cardiovascular disease, or their products (e.g., Lipoprotein(a) gene and its gene product), as well as genes or their products that are directly or indirectly regulated by or interact with these genes or their products, can be targeted for the development of therapeutic agents to treat cardiovascular disease, or prevent or delay onset of symptoms associated with cardiovascular disease. Therapeutic agents may comprise one or more of, for example, small non-protein and non-nucleic acid molecules, proteins, peptides, protein fragments, nucleic acids (DNA, RNA), PNA (peptide nucleic acids), or their derivatives or mimetics which can modulate the function and/or levels of the target genes or their gene products.

The nucleic acids and/or variants of the invention, or nucleic acids comprising their complementary sequence, may be used as antisense constructs to control gene expression in cells, tissues or organs. The methodology associated with antisense techniques is well known to the skilled artisan, and is described and reviewed in AntisenseDrug Technology: Principles, Strategies, and Applications, Crooke, ed., Marcel Dekker Inc., New York (2001). In general, antisense nucleic acid molecules are designed to be complementary to a region of mRNA expressed by a gene, so that the antisense molecule hybridizes to the mRNA, thus blocking translation of the mRNA into protein. Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Lavery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002)

The variants described herein can be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention (markers and/or haplotypes) can be inhibited or blocked. In one embodiment, the antisense molecules are designed to specifically bind a particular allelic form (i.e., one or several variants (alleles and/or haplotypes)) of the target nucleic acid, thereby inhibiting translation of a product originating from this specific allele or haplotype, but which do not bind other or alternate variants at the specific polymorphic sites of the target nucleic acid molecule.

As antisense molecules can be used to inactivate mRNA so as to inhibit gene expression, and thus protein expression, the molecules can be used to treat a disease or disorder, such as a cardiovascular disease. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants of the present invention (e.g., the markers and haplotypes set forth in Tables 3, 4 and 5) can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Lavery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9:R440-442 (1999).

A genetic defect leading to increased predisposition or risk for development of a cardiovascular disease, or a defect causing the disease, may be corrected permanently by administering to a subject carrying the defect a nucleic acid fragment that incorporates a repair sequence that supplies the normal/wild-type nucleotide(s) at the site of the genetic defect. Such site-specific repair sequence may concompass an RNA/DNA oligonucleotide that operates to promote endogenous repair of a subject's genomic DNA. The administration of the repair sequence may be performed by an appropriate vehicle, such as a complex with polyethelenimine, encapsulated in anionic liposomes, a viral vector such as an adenovirus vector, or other pharmaceutical compositions suitable for promoting intracellular uptake of the adminstered nucleic acid. The genetic defect may then be overcome, since the chimeric oligonucleotides induce the incorporation of the normal sequence into the genome of the subject, leading to expression of the normal/wild-type gene product. The replacement is propagated, thus rendering a permanent repair and alleviation of the symptoms associated with the disease or condition.

The present invention provides methods for identifying compounds or agents that can be used to treat cardiovascular diseases, such as coronary artery disease and myocardial infarction. Thus, the variants of the invention are useful as targets for the identification and/or development of therapeutic agents. In certain embodiments, such methods include assaying the ability of an agent or compound to modulate the activity and/or expression of a nucleic acid that includes at least one of the variants (markers and/or haplotypes) of the present invention, or the encoded product of the nucleic acid (e.g., the Lp(a) gene). This in turn can be used to identify agents or compounds that inhibit or alter the undesired activity or expression of the encoded nucleic acid product. Assays for performing such experiments can be performed in cell-based systems or in cell-free systems, as known to the skilled person. Cell-based systems include cells naturally expressing the nucleic acid molecules of interest, or recombinant cells that have been genetically modified so as to express a certain desired nucleic acid molecule.

Variant gene expression in a patient can be assessed by expression of a variant-containing nucleic acid sequence (for example, a gene containing at least one variant of the present invention, which can be transcribed into RNA containing the at least one variant, and in turn translated into protein), or by altered expression of a normal/wild-type nucleic acid sequence due to variants affecting the level or pattern of expression of the normal transcripts, for example variants in the regulatory or control region of the gene. Assays for gene expression include direct nucleic acid assays (mRNA), assays for expressed protein levels, or assays of collateral compounds involved in a pathway, for example a signal pathway. Furthermore, the expression of genes that are up- or down-regulated in response to the signal pathway can also be assayed. One embodiment includes operably linking a reporter gene, such as luciferase, to the regulatory region of the gene(s) of interest.

Modulators of gene expression can in one embodiment be identified when a cell is contacted with a candidate compound or agent, and the expression of mRNA is determined. The expression level of mRNA in the presence of the candidate compound or agent is compared to the expression level in the absence of the compound or agent. Based on this comparison, candidate compounds or agents for treating cardiovascular disease can be identified as those modulating the gene expression of the variant gene. When expression of mRNA or the encoded protein is statistically significantly greater in the presence of the candidate compound or agent than in its absence, then the candidate compound or agent is identified as a stimulator or up-regulator of expression of the nucleic acid. When nucleic acid expression or protein level is statistically significantly less in the presence of the candidate compound or agent than in its absence, then the candidate compound is identified as an inhibitor or down-regulator of the nucleic acid expression.

The invention further provides methods of treatment using a compound identified through drug (compound and/or agent) screening as a gene modulator (i.e. stimulator and/or inhibitor of gene expression).

### Methods of Assessing Probability of Response to Therapeutic Agents and Methods, Methods of Monitoring Treatment Progress and Methods for Treating Cardiovascular Disease

As is known in the art, individuals can have differential responses to a particular therapy (e.g., a therapeutic agent or therapeutic method). Pharmacogenomics addresses the issue of how genetic variations (e.g., the variants (markers and/or haplotypes) of the present invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the present invention), or therapeutic failure of the drug. Therefore, the variants of the present invention may determine the manner in which a therapeutic agent and/or method acts on the body, or the way in which the body metabolizes the therapeutic agent.

Accordingly, in one embodiment, the presence of a particular allele of a polymorphic marker, or the presence of a haplotype as described herein is indicative of a different response rate to a particular treatment modality for coronary artery disease or myocardial infarction. This means that a patient diagnosed with coronary artery disease or myocardial infarction, or at risk for coronary artery disease or myocardial infarction, and carrying a certain allele at a polymorphic or haplotype of the present invention (e.g., the at-risk alleles and/or haplotypes of the invention) would respond better to, or worse to, a specific therapeutic, drug and/or other therapy used to treating these diseases. Therefore, the presence or absence of the marker allele or haplotype could aid in deciding what treatment is appropriate for the patient. For example, for a newly diagnosed patient, the presence of a marker or haplotype of the present invention may be assessed (e.g., through testing DNA derived from a blood sample or other sample containing genomic DNA, as described herein). If the patient is positive for a marker allele or haplotype at (that is, at least one specific allele of the marker, or haplotype, is present), then the physician recommends one particular therapy (e.g., one particular therapeutic agent or a combination of therapeutic agents), while if the patient is negative for the at least one allele of a marker, or a haplotype, then a different course of therapy may be recommended (which may include recommending that no immediate therapy, other than serial monitoring for progression of the disease, be performed). Thus, the patient's carrier status could be used to help determine whether a particular treatment modality should be administered. The value lies within the possibilities of being able to diagnose the disease at an early stage and provide information to the clinician about prognosis/aggressiveness of the disease in order to be able to apply the most appropriate treatment.

As one example, the application of a genetic test can identify subjects who are at high risk of developing restenosis after coronary stent procedure. While it is know that some treatment methods for coronary artery disease, such as introducing drug-eluting stents and brachytherapy, are associated with decreased risk of in-stent restenosis, the use of these methods are restricted because of number of reasons, including economical reasons. Identification of individuals within the group of those undergoing coronary stent procedure who are carriers of particular genetic risk variants will allow targeting of those individuals that would benefit most from therapy associated with decreased risk of in-stent restenosis.

The present invention also relates to methods of monitoring effectiveness of a treatment for coronary artery disease and/or myocardial infarction. This can be done based on the genotype and/or haplotype status of the markers and haplotypes of the present invention, or by monitoring expression of genes that are associated with the variants (markers and haplotypes) of the present invention (e.g., the Lp(a) gene). The risk gene mRNA or the encoded polypeptide can be measured in a tissue sample (e.g., a peripheral blood sample, or a biopsy sample). Expression levels and/or mRNA levels can thus be determined before and during treatment to monitor its effectiveness. Alternatively, or concomitantly, the genotype and/or haplotype status of at least one risk variant for coronary artery disease or myocardial infarction as presented herein is determined before and during treatment to monitor its effectiveness.

The treatment modules of coronary artery disease and/or myocardial infarction to which the invention pertains includes, but is not limited to, methods of treatment for coronary artery disease or myocardial infarction or a susceptibility to coronary artery disease or myocardial infarction; methods of phophylaxis therapy for coronary artery disease or myocardial infarction; methods of treatment for acute coronary syndrome (e.g., unstable angina, non-ST-elevation myocardial infarction (NSTEMI) or ST-elevation myocardial infarction (STEMI)); methods for reducing risk of coronary artery disease and/or myocardial infarction; methods for decreasing risk of a second myocardial infarction; methods of treatment for atherosclerosis, such as for patients requiring treatment (e.g., angioplasty, stents, revascularization procedure) to restore blood flow in arteries (e.g., coronary, carotid, and/or femoral arteries); and/or methods for decreasing leukotriene synthesis (e.g., for treatment of coronary artery disease or myocardial infarction).

Treatment of coronary artery disease and myocardial infarction (MI) may be categorized as (i) preventive treatment and (ii) disease management. The main goal of the latter is to minimize damage to the heart and prevent further complications. The first line of disease management typically includes one or more of administration of oxygen, aspirin, glyceryl nitrate (nitroglycerin) and analgesia, such as morphine or related drugs. Once diagnosis of MI is made, additional therapy may include beta blockers, anticoagulation agents, including heparin and/or low molecular weigh heparin, and possibly also antiplatelet agents, such as clopidogrel. Secondary prevention, i.e. the management of risk of a recurrent MI, typically includes one or more of the following: Antiplatelet drug therapy, including aspirin and/or clopidogrel, beta blocker therapy, including metoprolol and carvedilol, ACE inhibitor therapy, Statin therapy, Aldosterone antagonist therapy, including eplerenone. Further, non-therapeutic administration of food supplements such as omega-3 fatty acids may be beneficial.

New preventive therapy for coronary artery disease and/or myocardial infarction, includes agents that act on the formation and/or rupture of plaques, and also includes phosphodiesterase inhibitors. Such therapeutic agents are useful in the methods of the invention, as described herein. This includes, but is not limited to, agents that target the leukotriene synthesis pathway. The leukotriene synthesis inhibitor can be any agent that inhibits or antagonizes a member of the leukotriene synthesis pathway (e.g., FLAP, 5-LO, LTC4S, LTA4H, and LTB4DH). For example, the leukotriene synthesis inhibitor can be an agent that inhibits or antagonizes FLAP polypeptide activity (e.g., a FLAP inhibitor) and/or FLAP nucleic acid expression (e.g., a FLAP nucleic acid antagonist). In another embodiment, the leukotriene synthesis inhibitor is an agent that inhibits or antagonizes polypeptide activity and/or nucleic acid expression of another member of the leukotriene biosynthetic pathway (e.g., LTC4S, LTA4H) or that increases breakdown of leukotrienes (e.g., LTB4DH). In preferred embodiments, the agent alters activity and/or nucleic acid expression of FLAP, LTA4H or of 5-LO. Preferred agents include those set forth in the Agent Table I herein. In another embodiment, preferred agents can be: 1-((4-chlorophenyl)methyl)-3-((1,1-dimethylethyl)thio)-alpha,alpha-dimethyl-5-(2-quinolinylmethoxy)-1H-Indole-2-propanoic acid otherwise known as MK-0591, (R)-(+)-alpha-cyclopentyl-4-(2-quinolinylmethoxy)-Benzeneacetic acid, otherwise known as BAY-x-1005, 3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid otherwise known as A-81834; or can be zileuton, atreleuton, 6-((3-fluoro-5-(tetrahydro-4-methoxy-2H-pyran-4yl)phenoxy)methyl)-1-methyl-2(1H)-quinlolinone otherwise known as ZD-2138, 1-((4-chlorophenyl)methyl)-3-((1,1-dimethylethyl)thio)-alpha,alpha-dimethyl-5-(2-quinolinylmethoxy)-1H-Indole-2-propanoic acid otherwise known as MK-886, 4-(3-(4-(2-Methyl-imidazol-1-yl)-phenylsulfanyl)-phenyl)-tetrahydro-pyran-4-carboxylic acid amide otherwise known as CJ-13610. Additional agents include those described in Penning et al., Med Chem. 2002 45(16):3482-90, Penning, Curr Pharm Des. 2001, 7(3):163-79 and Penning et al., J Med Chem. 2000 43(4):721-35. In another embodiment, the agent alters metabolism or activity of a leukotriene (e.g., LTA4, LTB4, LTC4, LTD4, LTE4, Cys LT1, Cys LT2), such as leukotriene antagonists or antibodies to leukotrienes, as well as agents which alter activity of a leukotriene receptor (e.g., BLT1, BLT2, CysLTR1, and CysLTR2).

In other preferred embodiments, the agent alters activity and/or nucleic acid expression of LTA4H. Preferred agents include those set forth in the Agent Table II; but also include the following agents: 1-[2-[4-(phenylmethyl)phenoxy]ethyl]-2-methyl-4-tetrazolylpieridine; 1-[2-[4-(4-oxazolyl)phenoxy)phenoxy]ethyl]pyrrolidine; 3-[methyl[3-[4-(2-thienylmethyl)phenoxy]propyl]amino]propionic acid; methyl 3-[methyl[3-[4-(2-thienylmethyl)phenoxy]propyl]amino]propionate; 3-[methyl[3-[4-(3-thienylmethyl)phenoxy]propyl]amino]propionic acid; methyl-3-[methyl[3-4-(3-thienylmethyl)phenoxy]propyl]amino]propionate; 3-[methyl[3-[4-(4-fluorophenoxy)phenoxy]propyl]amino]propionic acid; 3-[methyl[3-[4-(4-biphenyloxy)phenoxy]propyl]amino]propionic acid; N-[3-[[3-[4-(phenylmethyl)phenoxy]propyl]methylamino]propionyl]benzenesulfonamide; 1-[2-[4-(phenylmethyl)phenoxy]ethyl]-2-methyl-4-(1H-tetrazol-5-yl)piperidine; 1-[2-[4-(phenylmethyl)phenoxy]ethyl]-4-(1H-tetrazol-5-yl)piperidine. In another embodiment, preferred agents can be: ethyl-1-[2-[4-(phenylmethyl)phenoxy]ethyl]-4-piperidine-carboxylate, otherwise known as SC-56938; [4-[5-(3-Phenyl-propyl)thiophen-2-yl]butoxy]acetic acid, otherwise known as RP64966; (R)—S-[[4-(dimethylamino)phenyl]methyl]-N-(3-mercapto-2methyl-1-oxopropyl-L-cycteine, otherwise known as SA6541. In one preferred embodiment, the therapeutic agent is 4-{(S)-2-[4-(4-Chloro-phenoxy)-phenoxymethyl]-pyrrolidin-1-yl}-butyramide, also known as DG-051.

The agents for treating or preventing coronary artery disease and/or myocardial infarction can be adminstered alone, or in combination with a statin. Statins include, but are not limited to, the agents rovuvastatin, fluvastatin, atorvastatin, lovastatin (also known as mevolin), simvastatin, pravastatin, pitavastatin, mevastatin, crevastatin, ML-236A, ML-236B, MBV-530A and MB-530B.

All agents listed in the above and in Agent Table I and Agent Table II also include their optically pure enantiomers, salts, chemical derivatives, and analogues.

AGENT TABLE I

| | | | | | Date Patent | |
|---|---|---|---|---|---|---|
| | | | | | Issued/ | |
| | Product_Name | | | | Application | |
| | (Code) | Structure | Chemical Name | Patent Ref | Published | MOA |
| Abbott | atreleuton (ABT-761) | 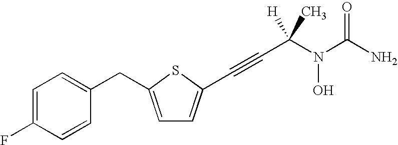 | (R)-(+)-N-[3[5-[(4- fluorophenyl)methyl]-2thienyl]- 1 methyl-2-propynyl]-N- hydroxurea | U.S. Pat. No. 5,288,751, U.S. Pat. No. 5,288,743, U.S. Pat. No. 5,616,596 | Feb. 22, 1994 Apr. 1, 1997 | 5-LPO inhibitor |
| Abbott | A-81834 | 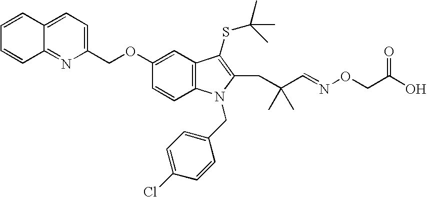 | 3-(3-(1,1-dimethylethylthio-5- 3-(quinoline-2-ylmethoxy)-1-(4- chloromethylphenyl)indo1e-2-yl) 2,2-dirnethylpropionaldehyde oxime-0-2-acetic acid | WO9203132, U.S. Pat. No. 5,459,150 | Mar. 5, 1992, Oct. 17, 1995 | FLAP Inhibitor |
| Abbott | A-86886 | 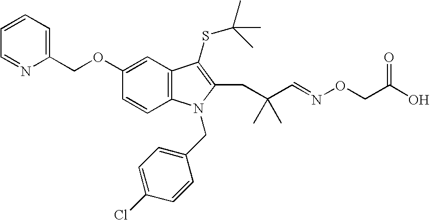 | 3-(3-(1,1-dirnethylethylthlo-5- (pyridin-2-ylmathoxy)-1-(4- chloromethylphenyl)indole-2-yl)- 2,2-dimethylpropionaldehyde oxime-0-2-acetic acid | W052031 32, U.S. Pat. No. 5,459,150 | Mar. 5,1992, Oct. 17, 1995 | 5-LP0 Inhibitor |
| Abbott | A-93178 | 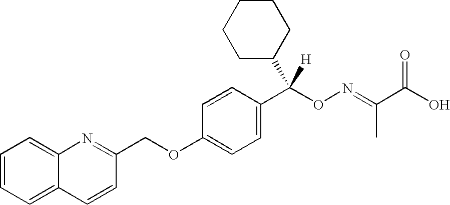 | | | | FLAP inhibitor |
| AstraZeneca | | 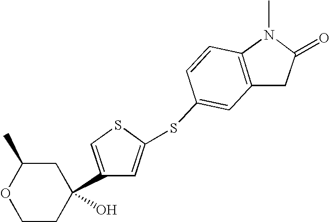 | | EP 623514 | Sep. 11, 1994 | 9-LPO inhibitor |
| AstraZenaca | ZD2138 | 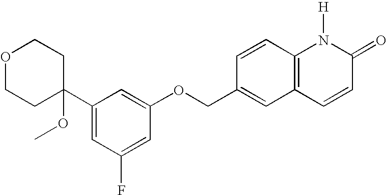 | 6-((3-f1uoro-5- (tetrahydro-4-methoxy-2H- pyran-4yl)phenoxy)methyl) 1-methyl-2(1H)-quinlolinone (alternatively NH can be N-methyl) | EP 466452 | | 5-LPO inhibitor |
| Bayer | BAY-X-1005 | 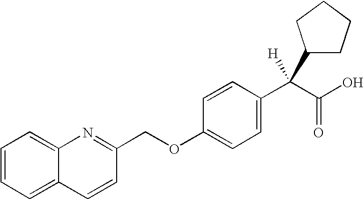 | (R)-(+)-alpha-cyclopentyl 4-(2-quinolinylmethoxy)- Benzeneacetic acid | U.S. Pat. No. 4,970,215 EP 344519, DE 19880531 | | FLAP Inhibitor |
| Merck | MK-0591 | 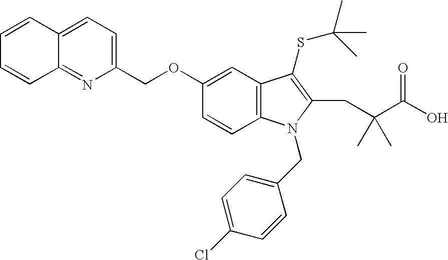 | 1-((4-chlorophenyl)methyl)-3- ((1,1-dimethylethyl)thio)alpha, alpha-dimethyl-5-(2-quinolinylmethoxy)-1H-Indole-2-propanoic acid | EP 419049, U.S. Pat. No. 19,890,822 | | FLAP Inhibitor |
| Merck | MK-866 | | (3(3-)-4-chlorobenzyl)-3-t-butyl- thio-5-isopropylindol-2-yl]2,2- dimethyl-propionic acid | | | 5-LPO inhibitor |
| Merck | MK-886 | 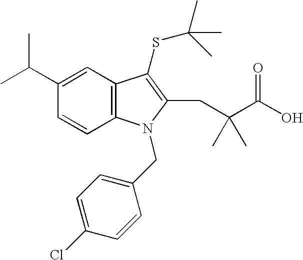 | 1-((4- chlorophenyl)methyl)-3- ((1,1dimethylethyl)thio)- alpha, alpha-dimethyl-5-(2-quinolinylmethoxy)-1H- Indole-2-propanoic acid | EP 419049, U.S. Pat. No. 19,890,822 | | 5-LPO inhibitor |
| Pfizer | CJ-13610 | | 4-(3-(4-(2-Methyl-imidazol-1-yl)- phenylsulfanyl)-phenyl)-tetrahydro-pyran-4- carboxylic acid amide | | | 5-LPO inhibitor |

AGENT TABLE II

| | Compound | | |
|---|---|---|---|
| Target | ID | Chemical Name | Patent/Reference |
| LTA4H | SC-57461A | 3-[methyl[3-[4- | Penning, T.D. et. al. Bioorg Med. |
| Inhibitor | | (phenylmethyl)phenoxy]- | Chem. Letters (2003), 13, 1137-1139. |
| | | propyl]amino]propionic | ibid, (2002), 12, 3383-3386 |
| | | acid | |
| LTA4H | SC-56938 | Ethyl-1-[2-[4- | Penning, T.D. et. al. Bioorg Med. |
| Inhibitor | | (phenylmethyl)phenoxy]ethyl]- | Chem. Letters (2003), 13, 1137-1139. |
| | | 4-piperidine- | ibid, (2002), 12, 3383-3386. |
| | | carboxylate | U.S. Pat. No. 6,506,87 6A1 |
| LTA4H | RP 64966 | [4-[5-(3-Phenyl- | WO9627585 |
| Inhibitor | | propyl)thiophen-2- | |
| | | yl]butoxy]acetic acid | |
| LTA4H | SA 6541 | (R)—S-[[4- | WO9809943 |
| Inhibitor | | (dimethylamino)phenyl]methyl]- | |
| | | N-(3-mercapto- | |
| | | 2methyl-1-oxopropyl-L- | |
| | | cycteine | |
| LTA4H | SA-9499/ | (R)-3-(4-Dimethylamino- | |
| Inhibitor | SA-6541 | benzylsulfanyl)-2-((R)-3- | |
| | | mercapto-2-methyl- | |
| | | propionylamino)-propionic | |
| | | acid | |
| LTB4 | Amelubant/ | Carbamic acid,((4-((3-((4- | U.S. Pat. No. 6,576,669 |
| Receptor | BIIL-284 | (1-(4-hydroxyphenyl)-1- | |
| Antagonist | | methylethyl)phenoxy)methyl)phenyl)methoxy)- | |
| | | phenyl)iminomethyl)ethyl ester | |
| LTB4 | BIRZ-227 | 5-Chloro-2-[3-(4- | Journal of Organic Chemistry |
| Receptor | | methoxy-phenyl)-2- | 1998, 63: 2(326-330). |
| Antagonist | | pyridin-2-yl-pyrrolidin-1- | |
| | | yl]-benzooxazole | |
| LTB4 | CP 195543 | 2-[(3S,4R)-3,4-dihydro-4- | Process: WO 98/11085 1998, priority |
| Receptor | | hydroxy-3- | US 60/26372 1996; J. |
| Antagonist | | (phenylmethyl)-2H-1- | Pharamacology and Expert. Therapy, |
| | | benzopyran-7-yl]-4- | 1998, 285: 946-54 |
| | | (trifluoromethyl)benzoic | |
| | | acid | |
| LTB4 | Ebselen | 2-Phenyl- | Journal of Cerebral Blood Flow and |
| Receptor | | benzo[d]isoselenazol-3- | Metabolism 1995, July 2-6 (S162); |
| Antagonist | | one | Drugs of the Future 1995, 20: 10 |
| | | | (1057) |
| LTB4 | LTB 019; | 4-[5-(4-Carbamimidoyl- | ACS Meeting 1994, 207th: San Diego |
| Receptor | CGS- | phenoxy)-pentyloxy]-N,N- | (MEDI 003); International Congress |
| Antagonist | 25019C | diisopropyl-3-methoxy- | of the Inflammation Research |
| | | benzamide maleate | Association 1994, 7th: White Haven |
| | | | (Abs W23) |
| LTB4 | LY 210073 | 5-(2-Carboxy-ethyl)-6-[6- | J Med Chem 1993 36 (12) 1726-1734 |
| Receptor | | (4-methoxy-phenyl)-hex- | |
| Antagonist | | 5-enyloxy]-9-oxo-9H- | |
| | | xanthene-2-carboxylic | |
| | | acid | |
| LTB4 | LY 213024 | 5-(3-carboxybenzoyl)-2- | J Med Chem 1993 36 (12) 1726-1734 |
| Receptor | | (decyloxy)benzenepropanoic | |
| Antagonist | | acid | |
| LTB4 | LY 255283 | 1-[5-ethyl-2-hydroxy-4- | EP 276064 B 1990, priority US 2479 |
| Receptor | | [[6-methyl-6-(1H- | 1987 |
| Antagonist | | tetrazol-5- | |
| | | yl)heptyl]oxy]phenyl]ethanone | |
| LTB4 | LY 264086 | 7-carboxy-3-(decyloxy)-9- | U.S. Pat. No. 4,996,230 1991, priority US 481413 |
| Receptor | | oxo-9H-xanthene-4- | 1990 |
| Antagonist | | propanoic acid | |
| LTB4 | LY 292728 | 7-carboxy-3-[3-[(5-ethyl- | EP 743064 A 1996, priority US |
| Receptor | | 4′-fluoro-2-hydroxy[1,1′- | 443179 1995 |
| Antagonist | | biphenyl]-4-yl)oxy]propoxy]- | |
| | | 9-oxo-9H- | |
| | | xanthene-4-propanoic acid | |
| | | disodium salt | |
| LTB4 | LY-293111 | Benzoic acid,2-(3-(3-((5- | Proceedings of the American Society |
| Receptor | (VML-295) | ethyl-4′-fluoro-2- | for Clinical Oncology 2002, 21: 1 |
| Antagonist | | hydroxy(1,1′-biphenyl)-4- | (Abs 343) [LY-293111 for Cancer] |
| | | yl)oxy)propoxy)-2- | SCRIP World Pharmaceutical News |
| | | propylphenoxy)- | 1997, 2272 (13) [for VML-295] |
| LTB4 | ONO 4057; | (E)-2-(4-carboxybutoxy)- | EP 405116 A 1991 |
| Receptor | LB 457 | 6-[[6-(4-methoxyphenyl)- | |
| Antagonist | | 5- | |
| | | hexenyl]oxy]benzenepropanoic | |
| | | acid | |
| LTB4 | PF 10042 | 1-[5-hydroxy-5-[8-(1- | EP 422329 B 1995, priority US |
| Receptor | | hydroxy-2-phenylethyl)-2- | 409630 1989 |
| Antagonist | | dibenzofuranyl]-1-oxo | |
| | | pentyl]pyrrolidine | |
| LTB4 | RG-14893 | 8-Benzyloxy-4-[(methyl- | SCRIP World Pharmaceutical News |
| Receptor | | phenethyl-carbamoyl)- | 1996, 2168 (20) |
| Antagonist | | methyl]-naphthalene-2- | |
| | | carboxylic acid | |
| LTB4 | SB-201993 | 3-{6-(2-Carboxy-vinyl)-5- | WO-09500487 |
| Receptor | | [8-(4-methoxy-phenyl)- | |
| Antagonist | | octyloxy]-pyridin-2- | |
| | | ylmethylsulfanylmethyl}- | |
| | | benzoic acid | |
| LTB4 | SC-52798 | 7-[3-(2- | Bioorganic and Medicinal Chemistry |
| Receptor | | Cyclopropylmethyl-3- | Letters 1994, 4: 6 (811-816); Journal |
| Antagonist | | methoxy-4-thiazol-4-yl- | of Medicinal Chemistry 1995, 38: 6 |
| | | phenoxy)-propoxy]-8- | (858-868) |
| | | propyl-chroman-2- | |
| | | carboxylic acid | |
| LTB4 | SC-53228 | 3-{7-[3-(2- | International Congress of the |
| Receptor | | Cyclopropylmethyl-3- | Inflammation Research Association |
| Antagonist | | methoxy-4- | 1994, 7th: White Haven (Abs W5) |
| | | methylcarbamoyl- | |
| | | phenoxy)-propoxy]-8- | |
| | | propyl-chroman-2-yl}- | |
| | | propionic acid | |
| LTB4 | WAY | 3-fluoro-4′-(2- | Drugs under Experimental and Clinical |
| Receptor | 121006 | quinolinylmethoxy)-[1,1′- | research 1991, 17: 8 (381-387) |
| Antagonist | | biphenyl]-4-acetic acid | |
| LTB4 | ZD-2138 | 3-Amino-3-(4-methoxy- | International Symposium on Medicinal |
| Receptor | | tetrahydro-pyran-4-yl)- | Chemistry 1994, 13th: Paris (P 197) |
| Antagonist | | acrylic acid 1-methyl-2- | |
| | | oxo-1,2-dihydro-quinolin- | |
| | | 6-ylmethyl ester | |

Alternatively, biological networks or metabolic pathways related to the markers and haplotypes of the present invention can be monitored by determining mRNA and/or polypeptide levels. This can be done for example, by monitoring expression levels or polypeptides for several genes belonging to the network and/or pathway, in samples taken before and during treatment. Alternatively, metabolites belonging to the biological network or metabolic pathway can be determined before and during treatment. Effectiveness of the treatment is determined by comparing observed changes in expression levels/metabolite levels during treatment to corresponding data from healthy subjects.

In a further aspect, the markers of the present invention can be used to increase power and effectiveness of clinical trials. Thus, individuals who are carriers of the at-risk variants of the present invention may be more likely to respond to a particular treatment modality. In one embodiment, individuals who carry at-risk variants for gene(s) in a pathway and/or metabolic network for which a particular treatment (e.g., small molecule drug, e.g. the small molecule drugs as listed in the above, e.g., the drugs listed in Agent Table I and Agent Table II) is targeting, are more likely to be responders to the treatment. In another embodiment, individuals who carry at-risk variants for a gene, which expression and/or function is altered by the at-risk variant, are more likely to be responders to a treatment modality targeting that gene, its expression or its gene product.

In a further aspect, the markers and haplotypes of the present invention can be used for targeting the selection of pharmaceutical agents for specific individuals. Personalized selection of treatment modalities, lifestyle changes (e.g., change in diet, exercise, weight loss program, smoking abstinence, less stressful lifestyle, etc.) or combination of the two, can be realized by the utilization of the at-risk variants of the present invention. Thus, the knowledge of an individual's status for particular markers of the present invention, can be useful for selection of treatment options that target genes or gene products affected by the at-risk variants of the invention. Certain combinations of variants may be suitable for one selection of treatment options, while other gene variant combinations may target other treatment options. Such combination of variant may include one variant, two variants, three variants, or four or more variants, as needed to determine with clinically reliable accuracy the selection of treatment module.

### Computer-Implemented Aspects

The present invention also relates to computer-implemented applications of the polymorphic markers and haplotypes described herein to be associated with coronary artery disease and myocardial infarction. Such applications can be useful for storing, manipulating or otherwise analyzing genotype data that is useful in the methods of the invention. One example pertains to storing genotype information derived from an individual on readable media, so as to be able to provide the genotype information to a third party (e.g., the individual), or for deriving information from the genotype data, e.g., by comparing the genotype data to information about genetic risk factors contributing to increased susceptibility to coronary artery disease and/or myocardial infarction, and reporting results based on such comparison.

One such aspect relates to computer-readable media. In general terms, such medium has capabilities of storing (i) identifier information for at least one polymorphic marker or a haplotype; (ii) an indicator of the frequency of at least one allele of said at least one marker, or the frequency of a haplotype, in individuals with coronary artery disease and/or myocardial infarction; and an indicator of the frequency of at least one allele of said at least one marker, or the frequency of a haplotype, in a reference population. The reference population can be a disease-free population of individuals. Alternatively, the reference population is a random sample from the general population, and is thus representative of the population at large. The frequency indicator may be a calculated frequency, a count of alleles and/or haplotype copies, or normalized or otherwise manipulated values of the actual frequencies that are suitable for the particular medium.

Additional information about the individual can be stored on the medium, such as ancestry information, information about sex, physical attributes or characteristics (including height and weight), biochemical measurements (such as blood pressure, blood lipid levels, lipid levels, such as cholesterol levels), biomarkers relevant for cardiovascular disease, as described further herein, or other useful information that is desirable to store or manipulate in the context of the genotype status of a particular individual.

The invention furthermore relates to an apparatus that is suitable for determination or manipulation of genetic data useful for determining a susceptibility to coronary artery disease and/or myocardial infarction in a human individual. Such an apparatus can include a computer-readable memory, a routine for manipulating data stored on the computer-readable memory, and a routine for generating an output that includes a measure of the genetic data. Such measure can include values such as allelic or haplotype frequencies, genotype counts, sex, age, phenotype information, values for odds ratio (OR) or relative risk (RR), population attributable risk (PAR), or other useful information that is either a direct statistic of the original genotype data or based on calculations based on the genetic data.

The markers and haplotypes shown herein to be associated with increased susceptibility (e.g., increased risk) of cardiovascular disease, are in certain embodiments useful for interpretation and/or analysis of genotype data. Thus in certain embodiments, an identification of an at-risk allele for coronary artery disease and/or myocardial infarction, as shown herein, or an allele at a polymorphic marker in LD with any one of the markers shown herein to be associated with coronary artery disease and/or myocardial infarction, is indicative of the individual from whom the genotype data originates is at increased risk of coronary artery disease and/or myocardial infarction. In one such embodiment, genotype data is generated for at least one polymorphic marker shown herein to be associated with coronary artery disease and/or myocardial infarction, or a marker in linkage disequilibrium therewith. The genotype data is subsequently made available to a third party, such as the individual from whom the data originates, for example via a user interface accessible over the internet, together with an interpretation of the genotype data, e.g., in the form of a risk measure (such as an absolute risk (AR), risk ratio (RR) or odds ration (OR)) for the cardiovascular disease. In another embodiment, at-risk markers identified in a genotype dataset derived from an individual are assessed and results from the assessment of the risk conferred by the presence of such at-risk variants in the dataset are made available to the individual, for example via a secure web interface, or by other communication means. The results of such risk assessment can be reported in numeric form (e.g., by risk values, such as absolute risk, relative risk, and/or an odds ratio, or by a percentage increase in risk compared with a reference), by graphical means, or by other means suitable to illustrate the risk to the individual from whom the genotype data is derived. In particular embodiments, the results of risk assessment is made available to a third party, e.g., a physician, other healthcare worker or genetic counselor.

### Markers Useful in Various Aspects of the Invention

The above-described applications can all be practiced with the markers and haplotypes of the invention that have in more detail been described with respect to methods of assessing susceptibility to cardiovascular disease and described in detail herein. Thus, these applications can in general be reduced to practice using any of the markers listed in Tables 1-5 herein, and markers in linkage disequilibrium therewith. In some embodiments, the markers are selected from the markers set forth in Table 3, and markers in linkage disequilibrium therewith. In some other embodiments, the markers are selected from the markers set forth in Table 4, and markers in linkage disequilibrium therewith. In some other embodiments, the markers are selected from the markers rs11751605 (SEQ ID NO:1), rs6076623 (SEQ ID NO:2), rs1412444 (SEQ ID NO:3), rs2163612 (SEQ ID NO:4), rs1029396 (SEQ ID NO:5), rs2243547 (SEQ ID NO:6), rs12534186 (SEQ ID NO:7), rs12134779 (SEQ ID NO:8) and rs7158073 (SEQ ID NO:9), and markers in linkage disequilibrium therewith. In some other embodiments, the markers are selected from the markers rs11751605 (SEQ ID NO:1), rs6076623 (SEQ ID NO:2), rs1412444 (SEQ ID NO:3), rs2163612 (SEQ ID NO:4), and rs1029396 (SEQ ID NO:5), and markers in linkage disequilibrium therewith. In some preferred embodiments, the marker is selected from rs11751605 (SEQ ID NO:1), and markers in linkage disequilibrium therewith. In other preferred embodiments, the marker is selected from rs6076623 (SEQ ID NO:2), and markers in linkage disequilibrium therewith. In yet other preferred embodiments, the marker is selected from rs1412444 (SEQ ID NO:3), and markers in linkage disequilibrium therewith. In other preferred embodiments, the marker is selected from rs2163612 (SEQ ID NO:4), and markers in linkage disequilibrium therewith. The marker may also be selected from rs1029396 (SEQ ID NO:5), and markers in linkage disequilibrium therewith. In a particularly preferred embodiment, the marker is rs11751605 (SEQ ID NO:1). In preferred embodiments, linkage disequilibrium between markers is defined by numerical values for r² of greater than 0.2.

In some embodiments, the presence of allele C in rs11751605, allele T in rs6076623, allele A in rs1412444, allele G in rs2163612 or allele G in rs1029396 is indicative of increased susceptibility of coronary artery disease or myocardial infarction in the individual who is undergoing testing, through analysis of genotype information (genotype dataset) derived from the individual, or through analysis of a sample comprising genomic DNA from the individual.

### Nucleic Acids and Polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a haplotype described herein). In one embodiment, the invention includes variants that hybridize under high stringency hybridization and wash conditions (e.g., for selective hybridization) to a nucleotide sequence that comprises the nucleotide sequence according to any one of SEQ ID NO:1-172 or a fragment thereof (or a nucleotide sequence comprising the complement of the nucleotide sequence of any one of SEQ ID NO:1-172), wherein the nucleotide sequence comprises at least one at-risk allele of at least one polymorphic marker, or at least one haplotype, as described herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S, and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20). Another example of an algorithm is BLAT (Kent, W. J. Genome Res. 12:656-64 (2002)).

The present invention also provides isolated nucleic acid molecules that contain a fragment or portion that hybridizes under highly stringent conditions to a nucleic acid that comprises, or consists of, the nucleotide sequence of any one of SEQ ID NO:1-172, or a nucleotide sequence comprising, or consisting of, the complement of the nucleotide sequence of any one of SEQ ID NO:1-172, wherein the nucleotide sequence comprises at least one polymorphic allele contained in the markers and haplotypes described herein. The nucleic acid fragments of the invention are at least about 15, at least about 18, 20, 23 or 25 nucleotides, and can be 30, 40, 50, 100, 200, 500, 1000, 10,000 or more nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule comprising a contiguous nucleotide sequence from any one of SEQ ID NO:1-172 and comprising at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques and the sequence information provided by the nucleotide sequence of any one of SEQ ID NO:1-172. See generally PCR Technology: Principles and Applications for DNA Amplification (ed. H.A. Erlich, Freeman Press, NY, N.Y., 1992); PCR Protocols: A Guide to Methods and Applications (Eds. Innis, et al., Academic Press, San Diego, Calif., 1990); Mattila, P. et al., Nucleic Acids Res., 19:4967-4973 (1991); Eckert, K. and Kunkel, T., PCR Methods and Applications, 1:17-24 (1991); PCR (eds. McPherson et al., IRL Press, Oxford); and U.S. Pat. No. 4,683,202, the entire teachings of each of which are incorporated herein by reference.

### Antibodies

Polyclonal antibodies and/or monoclonal antibodies that specifically bind one form of the gene product but not to the other form of the gene product (such as Lipoprotein(a), including kringle IV variants of Lipoprotein(a)) are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies may also be useful in pharmacogenomic analysis. In such embodiments, antibodies against variant proteins encoded by nucleic acids according to the invention (for example, variant forms of Lipoprotein(a), such as kringle IV repeat polymorphisms), such as variant proteins that are encoded by nucleic acids that contain at least one polymorphic marker of the invention, can be used to identify individuals that require modified treatment modalities.

Antibodies can furthermore be useful for assessing expression of variant proteins in disease states, such as in active stages of a cardiovascular disease, or in an individual with a predisposition to a disease related to the function of the protein, in particular a cardiovascular disease. Examples are provided by biomarker (e.g., cardiac markers), as described further herein. Antibodies specific for a variant protein of the present invention that is encoded by a nucleic acid that comprises at least one polymorphic marker or haplotype as described herein (e.g., CDKN2A and/or CDKN2B) can be used to screen for the presence of the variant protein, for example to screen for a predisposition to cardiovascular disease as indicated by the presence of the variant protein.

Antibodies can be used in other methods. Thus, antibodies are useful as diagnostic tools for evaluating proteins, such as variant proteins of the invention (e.g., variants of Lipoprotein(a), such as kringle IV repeat variants), in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. In the case where treatment is aimed at correcting the expression level or presence of the variant protein or aberrant tissue distribution or developmental expression of the variant protein, antibodies specific for the variant protein or fragments thereof can be used to monitor therapeutic efficacy.

Antibodies are further useful for inhibiting variant protein function (e.g., Lipoprotein(a)), for example by blocking the binding of a variant protein to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof may be increased by pegylation through conjugation to polyethylene glycol.

The present invention further relates to kits for using antibodies in the methods described herein. This includes, but is not limited to, kits for detecting the presence of a variant protein in a test sample. One preferred embodiment comprises antibodies such as a labelled or labelable antibody and a compound or agent for detecting variant proteins in a biological sample, means for determining the amount or the presence and/or absence of variant protein in the sample, and means for comparing the amount of variant protein in the sample with a standard, as well as instructions for use of the kit.

The present invention will now be exemplified by the following non-limiting examples.

### EXEMPLIFICATION

### Example 1

The following contains description of the identification of susceptibility factors found to be associated with coronary artery disease and myocardial infarction through single-point analysis of SNP markers and microsatellite markers.

### Methods

The study was approved by the Data Protection Commission of Iceland and the National Bioethics Committee.

### Icelandic Coronary Artery Disease Cohort

Over the last eight years individuals who have suffered an MI we have been recruited through cardiovascular disease (CVD) genetic programs at deCODE. Currently blood samples have been collected from 2525 MI patients. The individuals who had suffered an MI were identified from a registry of over 10,000 individuals who: a) had an MI before the age of 75 in Iceland in the years 1981 to 2002 and satisfy the MONICA criteria (J Clin Epidemiol 41, 105-14 (1988)); b) participated in a large prospective epidemiology study (1) done by the Icelandic Heart Association (IHA) over the past 30 years and had MI prior to 1981; c) had MI discharge diagnosis from the major hospitals in Reykjavik in the years 2003 and 2004. MI diagnoses of all individuals in the registry follow strict diagnostic rules based on signs, symptoms, electrocardiograms, cardiac enzymes and necropsy findings (2). The patients were contacted through collaborating physicians in the CVD genetic programs at deCODE. Most of the participants in the study visited the IHA and had their blood drawn, although participants who lived outside the Reykjavik area visited their local health care center.

Additional subjects with coronary artery disease, but are without known history of myocardial infarction, are identified from a list of those who have undergone coronary stent procedure in the major hospitals in Reykjavik in the years 1993 and 2003.

The controls used for the study were recruited as a part of various genetic programs at deCODE. The medical history for the controls were unknown unless if the control subjects also had participated in any of the CVD genetic programs (i.e. MI, stroke, peripheral vascular disease, type II diabetes, obesity, familial combined hyperlipidemia, coronary restenosis, and hypertension genetic programs). Individuals with known MI, stroke, peripheral vascular or coronary artery disease were excluded as controls.

Subjects from the United States
Cohort from Philadelphia

The study participants from Philadelphia were enrolled at the University of Pennsylvania Medical Center through the PENN CATH study program which studies the association of biochemical and genetic factors to coronary artery disease (CAD) in subjects undergoing cardiac catheterization. A total of 3850 subjects have participated. For the purpose of the current study we selected from the PENN CATH study individuals diagnosed with one of the following coronary artery disease: MI based on criteria for acute MI in terms of elevations of cardiac enzymes and electrocardiographic changes, or a self-reported history of MI, history of coronary artery bypass surgery (CABG) or percutaneous, transluminal coronary angioplasty (PTCA). To use as controls we selected individuals who were without significant luminal stenosis on coronary angiography (luminal stenosis less than 50%). Ethnicity information was self-reported.

The University of Pennsylvania Institutional Review Board approved the study and all subjects provided written informed consent.

Cohort from Durham, N.C.

The study participants were enrolled at Duke University Medical Center (Durham, N.C.) through the CATHGEN biorepository, recruited sequentially through the cardiac catheterization laboratories from 2001-2005. Biological samples and extensive clinical, angiographic, and longitudinal follow-up data were collected on all subjects consenting to participation. For purposes of this study, cases of MI were defined as those having a history of MI (by self-report and corroborated by review of medical records), or having suffered an MI during the study follow-up period. Controls from this cohort, defined as those with no history of MI prior or subsequent to the index cardiac catheterization, as well as no history of percutaneous or surgical coronary revascularization procedure; no subsequent percutaneous or surgical coronary revascularization procedures; ejection fraction on left ventriculogram greater than 40%; and no or minimal CAD on coronary angiography (defined as a CAD index less than or equal to 23 and no coronary vessel with clinically significant CAD (stenosis greater than 50%)). This study was approved by the Duke University Medical Center Institutional Review Board on Human Subjects and all subjects gave written informed consent.

Cohort from Atlanta

The study participants were enrolled at the Emory University Hospital, the Emory Clinic and Grady Memorial Hospitals through its Emory Genebank study and Clinical Registry in Neurology (CRIN). The Emory Genebank studies the association of biochemical and genetic factors with CAD in subjects undergoing cardiac catheterization. For the purpose of the current study those subjects who had a self-reported history of MI, CABG, or PTCA, were selected and used as a patient group. Control subjects were selected from a group of individuals with non-vascular neurological diseases (mainly Parkinson's and Alzheimer's diseases) recruited from CRIN, their spouses, unrelated friends and community volunteers. These subjects were matched for age, and ethnicity to the patient population. Controls were excluded if they had a known history of MI or coronary artery disease. All subjects provided written informed consent. Information on ethnicity was self-reported.

### Genotyping

A genome-wide scan of 1700 Icelandic individuals diagnosed with myocardial infarction (MI), 2034 patients with coronary artery disease and 10635 population controls was performed using Infinium HumanHap300 SNP chips from Illumina for assaying approximately 317,000 single nucleotide polymorphisms (SNPs) on a single chip (Illumina, San Diego, Calif., USA). SNP genotyping for replication in other case-control cohorts was carried using the Centaurus platform (Nanogen).

### Statistical Methods for Association Analysis.

To test individual markers for association to disease phenotypes such as coronary artery disease or myocardial infarction, we use a likelihood ratio test to calculate a two-sided P-value for each allele of the markers. We calculate relative risk (RR) and population attributable risk (PAR) assuming a multiplicative model (C. T. Falk, P. Rubinstein, Ann Hum Genet 51 (Pt 3), 227 (1987); J. D. Terwilliger, J. Ott, Hum Hered 42, 337 (1992)). To elucidate the linkage disequilibrium between markers in the region we used the CEPH Caucasian HapMap data. We calculated LD between pairs of SNPs using the standard definition of D′ (R. C. Lewontin, Genetics 50, 757 (1964)) and for the correlation coefficient r² (W. G. Hill, A. Robertson, Genetics 60, 615 (November, 1968). For the Icelandic cohort, to take into account that some of the individuals are related to each other, we obtained the null statistic of the test statistic either by simulating genotypes through the Icelandic genealogy or from the test statistic for all the 300,000 tested for association in the initial genome-wide association scan (citation). Model-free estimates of the genotype relative risk are generated as follows: RR of genotype G₁ compared to genotype G₀ was estimated by [n(G₁)/n(G₀)]/[m(G₁)/m(G₀)] where n and m denote genotype counts in patients and controls respectively. Results from different cohorts were combined using a Mantel-Hanezel model (citation) where cohorts are allowed to have different population frequencies for the alleles/genotypes but assume to have common relative risks.

We use multiple regression to test for association between markers and quantitative traits, such as ago of onset of MI in the cases, where the number of copies of the at-risk variant carried by an individual is taken as explanatory variable and the quantitative trait as the response variable. The association is adjusted for age and gender, where appropriate, by including corresponding terms in the regression analysis as explanatory variables.

### Results

### Genome-Wide Association Study

We successfully genotyped 1700 Icelandic myocardial infarction patients, 2034 coronary artery disease Patients and 10635 population control individuals without known history of coronary artery disease (Cohort A) using the Illumina 330K chip. We performed a genome-wide scan for association to MI, testing individually each of the 309,091 SNPs that was successfully genotyped. Results of this association analysis is presented in Table 1, wherein markers showing significant association to MI are indicated (p-value less than 0.01). As can be seen in the table, a large number of markers show significant association to MI, including markers rs2227165 on chromosome X, rs12304836 on chromosome 12 and rs11225090 on chromosome 11.

The markers found to be associating with MI as presented in Table 1 may be useful for risk assessment and diagnostic purposes for MI, either alone or in combination. Even in the cases where the increase in risk by individual markers is relatively modest, i.e. on the order of 15-30%, the association may have significant implications. Thus, relatively common variants may have significant contribution to the overall risk (Population Attributable Risk is high), or combination of markers can be used to define groups of individual who, based on the combined risk of the markers, is at significant combined risk of developing MI.

TABLE 1
Association results for SNPs showing association to Myoardial Infarction (MI) and coronary artery disease (CAD). The columns indicate
(1) chromosome, (2) position in NCBI Build 34, (3) rs name of the SNP in question, (4), the associating allele, (5) frequency of the associating
allele in controls, (6) the number of controls, (7), the p-value for association to MI, (8) the Relative Risk of association to MI, (9) the frequency
of the associating allele in MI patients, (10) the number of MI patients, (11) the p-value of association to CAD, (12) the Relative Risk of
association to CAD, (13) the frequency of the associating allele in CAD patients, (14) the number of CAD patients. The table is sorted on p-
values for association to MI.

| chr | position b34 | rs-name | allele | f(con) | N(con) | p(MI) | RR(MI) | f(MI) | N(MI) | p(CAD) | rr(CAD) | f(CAD) | N(CAD) |
|---|---|---|---|---|---|---|---|---|---|---|---|---|---|
| CX | 91765264 | rs2227165 | C | 0.897 | 8178 | 2.08E−07 | 1.61 | 0.934 | 1068 | 5.16E−05 | 1.39 | 0.924 | 1276 |
| C12 | 124441592 | rs12304836 | C | 0.141 | 10541 | 7.66E−06 | 1.27 | 0.173 | 1689 | 6.26E−06 | 1.25 | 0.171 | 2020 |
| C11 | 101370325 | rs11225090 | T | 0.969 | 10513 | 8.71E−06 | 1.85 | 0.983 | 1646 | 2.95E−05 | 1.68 | 0.981 | 1979 |
| C05 | 158504417 | rs7442701 | T | 0.174 | 10629 | 1.05E−05 | 1.25 | 0.208 | 1699 | 5.90E−05 | 1.21 | 0.202 | 2033 |
| C12 | 116419694 | rs816204 | A | 0.851 | 10607 | 1.10E−05 | 1.29 | 0.881 | 1693 | 4.70E−05 | 1.25 | 0.877 | 2026 |
| C20 | 4131671 | rs6076623 | T | 0.364 | 10632 | 1.20E−05 | 1.19 | 0.406 | 1700 | 2.40E−06 | 1.19 | 0.406 | 2034 |
| C17 | 17995010 | rs4925125 | T | 0.347 | 10430 | 1.55E−05 | 1.19 | 0.388 | 1680 | 3.40E−04 | 1.15 | 0.379 | 2009 |
| C10 | 90667504 | rs1412444 | A | 0.356 | 10626 | 1.60E−05 | 1.19 | 0.397 | 1700 | 2.38E−04 | 1.15 | 0.389 | 2034 |
| C17 | 18235585 | rs854787 | C | 0.398 | 10568 | 1.72E−05 | 1.19 | 0.44 | 1689 | 2.63E−05 | 1.17 | 0.436 | 2023 |
| C10 | 90675056 | rs2243547 | G | 0.339 | 10200 | 1.82E−05 | 1.2 | 0.38 | 1642 | 3.87E−04 | 1.15 | 0.37 | 1970 |
| C08 | 506479 | rs722782 | C | 0.888 | 10607 | 2.44E−05 | 1.33 | 0.914 | 1668 | 1.72E−04 | 1.26 | 0.909 | 2002 |
| C11 | 82905504 | rs2163612 | G | 0.476 | 10625 | 3.12E−05 | 1.18 | 0.517 | 1698 | 3.92E−05 | 1.16 | 0.513 | 2032 |
| C01 | 62766259 | rs12134779 | T | 0.29 | 10634 | 3.21E−05 | 1.19 | 0.327 | 1699 | 1.81E−04 | 1.16 | 0.321 | 2033 |
| CX | 66228891 | rs7050085 | G | 0.909 | 8267 | 5.71E−05 | 1.45 | 0.936 | 1103 | 1.15E−04 | 1.39 | 0.933 | 1316 |
| C07 | 116846839 | rs12534186 | T | 0.079 | 10631 | 5.99E−05 | 1.31 | 0.102 | 1699 | 2.22E−04 | 1.26 | 0.098 | 2033 |
| C05 | 158510030 | rs7447732 | C | 0.316 | 10635 | 6.14E−05 | 1.18 | 0.353 | 1700 | 3.34E−04 | 1.15 | 0.347 | 2034 |
| C07 | 116800818 | rs2299445 | C | 0.079 | 10630 | 6.16E−05 | 1.31 | 0.101 | 1699 | 2.21E−04 | 1.27 | 0.098 | 2033 |
| C07 | 116800818 | rs2299445 | C | 0.079 | 10630 | 6.16E−05 | 1.31 | 0.101 | 1699 | 2.21E−04 | 1.27 | 0.098 | 2033 |
| C19 | 4229502 | rs4807567 | T | 0.419 | 10586 | 6.30E−05 | 1.17 | 0.458 | 1699 | 1.99E−04 | 1.15 | 0.453 | 2033 |
| C13 | 26936609 | rs4769613 | T | 0.467 | 10633 | 6.56E−05 | 1.17 | 0.506 | 1699 | 9.01E−06 | 1.18 | 0.507 | 2033 |
| C05 | 158671846 | rs4921437 | T | 0.212 | 10594 | 6.57E−05 | 1.21 | 0.245 | 1691 | 6.09E−04 | 1.16 | 0.238 | 2023 |
| C10 | 15439877 | rs12269612 | T | 0.398 | 10634 | 6.66E−05 | 1.17 | 0.437 | 1700 | 8.04E−04 | 1.13 | 0.428 | 2034 |
| C12 | 87995622 | rs1568383 | T | 0.097 | 10635 | 7.05E−05 | 1.28 | 0.121 | 1700 | 1.11E−04 | 1.25 | 0.119 | 2034 |
| C21 | 24038081 | rs2828495 | A | 0.211 | 10635 | 7.27E−05 | 1.2 | 0.244 | 1700 | 4.74E−05 | 1.19 | 0.242 | 2034 |
| C10 | 45239070 | rs2279434 | T | 0.065 | 10634 | 7.50E−05 | 1.34 | 0.085 | 1700 | 5.02E−04 | 1.27 | 0.081 | 2033 |
| C02 | 212588265 | rs12329252 | T | 0.676 | 10635 | 7.87E−05 | 1.18 | 0.712 | 1700 | 6.42E−04 | 1.14 | 0.705 | 2034 |
| C03 | 62420156 | rs9874646 | T | 0.709 | 10634 | 8.11E−05 | 1.19 | 0.744 | 1700 | 3.83E−04 | 1.16 | 0.738 | 2034 |
| C04 | 21568604 | rs4282162 | T | 0.528 | 10633 | 8.28E−05 | 1.17 | 0.567 | 1700 | 1.66E−04 | 1.15 | 0.563 | 2034 |
| C03 | 62402714 | rs1534725 | T | 0.782 | 10627 | 8.33E−05 | 1.21 | 0.814 | 1699 | 1.33E−04 | 1.19 | 0.811 | 2033 |
| C03 | 62402714 | rs1534725 | T | 0.782 | 10627 | 8.33E−05 | 1.21 | 0.814 | 1699 | 1.33E−04 | 1.19 | 0.811 | 2033 |
| C09 | 87419341 | rs3211683 | A | 0.088 | 10635 | 9.41E−05 | 1.29 | 0.111 | 1700 | 2.86E−04 | 1.25 | 0.108 | 2034 |
| C11 | 42877662 | rs1532096 | T | 0.861 | 10635 | 9.63E−05 | 1.26 | 0.887 | 1700 | 9.38E−05 | 1.24 | 0.885 | 2034 |
| C22 | 42582255 | rs738407 | A | 0.635 | 10634 | 9.70E−05 | 1.18 | 0.672 | 1700 | 5.67E−04 | 1.14 | 0.665 | 2034 |
| C17 | 17947855 | rs3183702 | T | 0.349 | 10630 | 9.76E−05 | 1.17 | 0.386 | 1699 | 1.01E−03 | 1.13 | 0.378 | 2033 |
| C02 | 39191408 | rs963731 | C | 0.951 | 10365 | 1.01E−04 | 1.49 | 0.966 | 1651 | 1.21E−04 | 1.44 | 0.965 | 1978 |
| C09 | 87414699 | rs3211650 | T | 0.089 | 10634 | 1.02E−04 | 1.29 | 0.111 | 1700 | 3.09E−04 | 1.25 | 0.108 | 2034 |
| C06 | 71671372 | rs7753765 | A | 0.144 | 10633 | 1.03E−04 | 1.23 | 0.172 | 1700 | 2.32E−04 | 1.2 | 0.169 | 2034 |
| C01 | 30378256 | rs593993 | C | 0.834 | 10626 | 1.09E−04 | 1.24 | 0.862 | 1697 | 7.25E−05 | 1.23 | 0.86 | 2031 |
| C11 | 17756131 | rs7946133 | T | 0.915 | 10540 | 1.12E−04 | 1.35 | 0.936 | 1641 | 1.43E−04 | 1.31 | 0.934 | 1968 |
| C02 | 212605929 | rs17406681 | T | 0.275 | 10635 | 1.13E−04 | 1.18 | 0.31 | 1700 | 3.42E−04 | 1.15 | 0.305 | 2034 |
| C02 | 212605929 | rs17406681 | T | 0.275 | 10635 | 1.13E−04 | 1.18 | 0.31 | 1700 | 3.42E−04 | 1.15 | 0.305 | 2034 |
| C03 | 14276852 | rs6772823 | G | 0.276 | 10580 | 1.18E−04 | 1.18 | 0.31 | 1675 | 1.98E−04 | 1.16 | 0.307 | 2008 |
| C17 | 17934326 | rs9902941 | C | 0.349 | 10634 | 1.25E−04 | 1.17 | 0.385 | 1700 | 1.18E−03 | 1.13 | 0.377 | 2034 |
| C02 | 123847245 | rs1395930 | T | 0.494 | 10633 | 1.25E−04 | 1.16 | 0.532 | 1698 | 3.18E−05 | 1.16 | 0.532 | 2032 |
| C05 | 158647626 | rs254850 | T | 0.215 | 10635 | 1.29E−04 | 1.19 | 0.247 | 1700 | 1.30E−03 | 1.15 | 0.24 | 2034 |
| C03 | 170405740 | rs756644 | A | 0.431 | 10633 | 1.30E−04 | 1.16 | 0.468 | 1700 | 1.56E−03 | 1.12 | 0.459 | 2034 |
| C12 | 47223943 | rs12581026 | C | 0.046 | 10634 | 1.33E−04 | 1.39 | 0.063 | 1700 | 3.67E−03 | 1.27 | 0.058 | 2034 |
| C17 | 17849989 | rs4925108 | C | 0.392 | 10623 | 1.35E−04 | 1.16 | 0.429 | 1700 | 4.28E−04 | 1.14 | 0.424 | 2034 |
| C17 | 17849989 | rs4925108 | C | 0.392 | 10623 | 1.35E−04 | 1.16 | 0.429 | 1700 | 4.28E−04 | 1.14 | 0.424 | 2034 |
| C12 | 95967869 | rs11108788 | G | 0.032 | 10618 | 1.38E−04 | 1.47 | 0.046 | 1698 | 3.43E−04 | 1.4 | 0.044 | 2032 |
| C01 | 53740224 | rs2294512 | G | 0.682 | 10635 | 1.41E−04 | 1.18 | 0.716 | 1700 | 1.47E−03 | 1.13 | 0.708 | 2034 |
| C05 | 102457857 | rs9327877 | T | 0.19 | 10634 | 1.45E−04 | 1.2 | 0.22 | 1700 | 2.47E−04 | 1.18 | 0.217 | 2034 |
| C04 | 21549446 | rs10516402 | C | 0.504 | 10633 | 1.46E−04 | 1.16 | 0.541 | 1700 | 2.09E−04 | 1.15 | 0.538 | 2034 |
| C04 | 21549446 | rs10516402 | C | 0.504 | 10633 | 1.46E−04 | 1.16 | 0.541 | 1700 | 2.09E−04 | 1.15 | 0.538 | 2034 |
| C20 | 15053882 | rs411944 | G | 0.645 | 10632 | 1.47E−04 | 1.17 | 0.68 | 1700 | 1.22E−03 | 1.13 | 0.673 | 2033 |
| C13 | 42581715 | rs9525923 | A | 0.208 | 10635 | 1.56E−04 | 1.19 | 0.239 | 1700 | 5.62E−04 | 1.16 | 0.234 | 2034 |
| C02 | 176947426 | rs921083 | G | 0.776 | 10633 | 1.56E−04 | 1.2 | 0.806 | 1700 | 2.93E−04 | 1.18 | 0.803 | 2034 |
| C05 | 97515007 | rs950195 | G | 0.63 | 10631 | 1.65E−04 | 1.17 | 0.665 | 1700 | 5.67E−05 | 1.17 | 0.665 | 2034 |
| C05 | 6897773 | rs870347 | T | 0.924 | 10635 | 1.73E−04 | 1.35 | 0.942 | 1700 | 1.14E−03 | 1.27 | 0.939 | 2034 |
| C14 | 67308303 | rs8003722 | A | 0.102 | 10634 | 1.73E−04 | 1.26 | 0.126 | 1700 | 2.73E−04 | 1.23 | 0.123 | 2034 |
| C05 | 50867460 | rs6449708 | T | 0.452 | 10611 | 1.73E−04 | 1.16 | 0.489 | 1693 | 1.17E−03 | 1.13 | 0.482 | 2027 |
| C05 | 50867460 | rs6449708 | T | 0.452 | 10611 | 1.73E−04 | 1.16 | 0.489 | 1693 | 1.17E−03 | 1.13 | 0.482 | 2027 |
| C06 | 160872518 | rs11751605 | C | 0.11 | 10632 | 1.75E−04 | 1.25 | 0.134 | 1700 | 3.87E−04 | 1.22 | 0.131 | 2033 |
| C02 | 123861662 | rs2661006 | T | 0.493 | 10623 | 1.76E−04 | 1.16 | 0.529 | 1696 | 5.58E−05 | 1.16 | 0.529 | 2030 |
| C13 | 45452894 | rs7322927 | A | 0.125 | 10635 | 1.78E−04 | 1.24 | 0.151 | 1700 | 3.29E−03 | 1.17 | 0.144 | 2034 |
| C05 | 56215087 | rs832540 | C | 0.415 | 10626 | 1.81E−04 | 1.16 | 0.451 | 1700 | 3.23E−04 | 1.14 | 0.447 | 2034 |
| C04 | 166320428 | rs11723570 | C | 0.511 | 10635 | 1.92E−04 | 1.16 | 0.547 | 1700 | 1.49E−04 | 1.15 | 0.545 | 2034 |
| C19 | 17027981 | rs2278997 | T | 0.229 | 10635 | 1.93E−04 | 1.19 | 0.261 | 1700 | 8.49E−04 | 1.15 | 0.255 | 2034 |
| C19 | 17027981 | rs2278997 | T | 0.229 | 10635 | 1.93E−04 | 1.19 | 0.261 | 1700 | 8.49E−04 | 1.15 | 0.255 | 2034 |
| C16 | 73532441 | rs12927636 | G | 0.105 | 10635 | 1.94E−04 | 1.25 | 0.129 | 1700 | 7.63E−04 | 1.21 | 0.125 | 2034 |
| C11 | 1371919 | rs7944761 | T | 0.526 | 10631 | 2.04E−04 | 1.16 | 0.563 | 1700 | 8.52E−04 | 1.13 | 0.557 | 2034 |
| C07 | 116887417 | rs975722 | G | 0.389 | 10635 | 2.05E−04 | 1.16 | 0.425 | 1700 | 3.18E−04 | 1.14 | 0.421 | 2034 |
| C07 | 116887417 | rs975722 | G | 0.389 | 10635 | 2.05E−04 | 1.16 | 0.425 | 1700 | 3.18E−04 | 1.14 | 0.421 | 2034 |
| C17 | 18035257 | rs6502622 | G | 0.359 | 10635 | 2.07E−04 | 1.16 | 0.395 | 1700 | 2.81E−03 | 1.12 | 0.386 | 2034 |
| C02 | 76284921 | rs443713 | G | 0.394 | 10635 | 2.09E−04 | 1.16 | 0.43 | 1700 | 1.77E−04 | 1.15 | 0.428 | 2034 |
| C09 | 16319537 | rs1888955 | A | 0.742 | 10632 | 2.19E−04 | 1.19 | 0.774 | 1700 | 8.61E−04 | 1.15 | 0.768 | 2034 |
| C09 | 16324761 | rs12683609 | A | 0.571 | 10635 | 2.20E−04 | 1.16 | 0.607 | 1699 | 4.83E−04 | 1.14 | 0.602 | 2033 |
| C05 | 167501721 | rs7736530 | G | 0.798 | 10634 | 2.20E−04 | 1.21 | 0.826 | 1700 | 2.77E−04 | 1.19 | 0.824 | 2034 |
| C15 | 24283869 | rs4572353 | T | 0.492 | 10632 | 2.25E−04 | 1.16 | 0.528 | 1700 | 4.64E−04 | 1.14 | 0.524 | 2034 |
| C07 | 116792948 | rs213977 | G | 0.409 | 10633 | 2.28E−04 | 1.16 | 0.445 | 1700 | 3.73E−04 | 1.14 | 0.441 | 2034 |
| C02 | 76346853 | rs1595968 | T | 0.394 | 10622 | 2.29E−04 | 1.16 | 0.43 | 1695 | 1.92E−04 | 1.15 | 0.428 | 2029 |
| C01 | 20544933 | rs12724604 | A | 0.036 | 10634 | 2.35E−04 | 1.42 | 0.051 | 1700 | 5.31E−05 | 1.43 | 0.051 | 2034 |
| C04 | 21483069 | rs1827591 | G | 0.535 | 10635 | 2.41E−04 | 1.16 | 0.571 | 1700 | 2.98E−04 | 1.14 | 0.568 | 2034 |
| C01 | 148957002 | rs12066445 | A | 0.299 | 10635 | 2.47E−04 | 1.17 | 0.332 | 1700 | 1.08E−03 | 1.14 | 0.326 | 2034 |
| C17 | 9590955 | rs3891720 | C | 0.869 | 10613 | 2.47E−04 | 1.25 | 0.893 | 1698 | 4.95E−04 | 1.22 | 0.89 | 2032 |
| C17 | 17943470 | rs4925119 | A | 0.347 | 10615 | 2.54E−04 | 1.16 | 0.381 | 1696 | 2.24E−03 | 1.12 | 0.373 | 2029 |
| C08 | 144065203 | rs7844961 | T | 0.09 | 10635 | 2.61E−04 | 1.27 | 0.112 | 1700 | 1.82E−03 | 1.21 | 0.107 | 2034 |
| C01 | 57254228 | rs2991515 | T | 0.479 | 10635 | 2.67E−04 | 1.15 | 0.515 | 1700 | 2.39E−05 | 1.17 | 0.518 | 2034 |
| C20 | 4150948 | rs1741315 | A | 0.483 | 10633 | 2.68E−04 | 1.15 | 0.519 | 1700 | 5.86E−04 | 1.13 | 0.514 | 2034 |
| C20 | 4150948 | rs1741315 | A | 0.483 | 10633 | 2.68E−04 | 1.15 | 0.519 | 1700 | 5.86E−04 | 1.13 | 0.514 | 2034 |
| C09 | 132592188 | rs4917341 | C | 0.481 | 10587 | 2.69E−04 | 1.15 | 0.516 | 1697 | 1.12E−03 | 1.13 | 0.51 | 2029 |
| C08 | 28215758 | rs4732849 | T | 0.273 | 10635 | 2.70E−04 | 1.17 | 0.306 | 1700 | 1.05E−02 | 1.11 | 0.294 | 2034 |
| C20 | 47199260 | rs981303 | A | 0.929 | 10623 | 2.73E−04 | 1.35 | 0.946 | 1696 | 1.53E−05 | 1.4 | 0.948 | 2029 |
| C10 | 84830992 | rs7921473 | G | 0.815 | 10635 | 2.77E−04 | 1.21 | 0.842 | 1700 | 3.79E−05 | 1.22 | 0.844 | 2034 |
| C15 | 43779321 | rs11630033 | C | 0.9 | 10606 | 2.77E−04 | 1.29 | 0.92 | 1694 | 3.81E−04 | 1.26 | 0.918 | 2028 |
| C20 | 13686900 | rs2026058 | T | 0.764 | 10634 | 2.77E−04 | 1.19 | 0.794 | 1700 | 3.63E−03 | 1.14 | 0.786 | 2034 |
| C21 | 16738666 | rs1349227 | T | 0.238 | 10635 | 2.77E−04 | 1.18 | 0.269 | 1700 | 4.65E−04 | 1.16 | 0.266 | 2034 |
| C07 | 116579323 | rs1029396 | G | 0.069 | 10634 | 2.78E−04 | 1.3 | 0.088 | 1700 | 2.26E−04 | 1.28 | 0.087 | 2034 |
| C11 | 7588325 | rs12292613 | T | 0.892 | 10509 | 2.88E−04 | 1.28 | 0.913 | 1670 | 3.35E−03 | 1.2 | 0.908 | 2003 |
| C08 | 70812569 | rs3829049 | G | 0.708 | 10635 | 2.91E−04 | 1.17 | 0.74 | 1700 | 5.19E−04 | 1.15 | 0.737 | 2034 |
| C03 | 25122677 | rs6763490 | C | 0.6 | 10635 | 2.94E−04 | 1.16 | 0.635 | 1700 | 7.39E−04 | 1.14 | 0.63 | 2034 |
| C17 | 17911836 | rs4925114 | A | 0.359 | 10615 | 2.97E−04 | 1.16 | 0.393 | 1697 | 1.89E−03 | 1.12 | 0.386 | 2031 |
| C01 | 19443039 | rs7535952 | A | 0.717 | 10510 | 2.99E−04 | 1.18 | 0.749 | 1660 | 3.29E−03 | 1.13 | 0.741 | 1990 |
| C07 | 46273364 | rs10243723 | G | 0.97 | 10628 | 3.04E−04 | 1.61 | 0.981 | 1699 | 1.27E−03 | 1.47 | 0.979 | 2033 |
| C08 | 18243855 | rs7006687 | T | 0.575 | 10632 | 3.05E−04 | 1.16 | 0.61 | 1698 | 7.18E−05 | 1.16 | 0.61 | 2032 |
| C16 | 61289938 | rs12924635 | G | 0.389 | 10380 | 3.06E−04 | 1.16 | 0.425 | 1624 | 3.29E−03 | 1.12 | 0.416 | 1953 |
| C04 | 10479642 | rs4697653 | G | 0.526 | 10604 | 3.08E−04 | 1.15 | 0.562 | 1699 | 8.82E−04 | 1.13 | 0.556 | 2032 |
| C12 | 112489807 | rs249047 | A | 0.614 | 10634 | 3.11E−04 | 1.16 | 0.648 | 1700 | 3.87E−03 | 1.12 | 0.639 | 2034 |
| C07 | 67324200 | rs12540706 | G | 0.311 | 10634 | 3.15E−04 | 1.16 | 0.344 | 1700 | 2.70E−03 | 1.12 | 0.337 | 2034 |
| C10 | 26191683 | rs7086207 | G | 0.404 | 10625 | 3.17E−04 | 1.15 | 0.439 | 1699 | 5.10E−04 | 1.14 | 0.435 | 2033 |
| C05 | 40281304 | rs13165359 | G | 0.877 | 10634 | 3.19E−04 | 1.26 | 0.899 | 1700 | 2.77E−03 | 1.19 | 0.894 | 2034 |
| C05 | 50777478 | rs10038238 | A | 0.567 | 10634 | 3.20E−04 | 1.15 | 0.602 | 1699 | 1.41E−03 | 1.13 | 0.596 | 2033 |
| C01 | 65319795 | rs2025805 | C | 0.565 | 10634 | 3.25E−04 | 1.15 | 0.6 | 1700 | 3.31E−04 | 1.14 | 0.597 | 2034 |
| C17 | 18204411 | rs854813 | C | 0.387 | 10632 | 3.25E−04 | 1.15 | 0.422 | 1700 | 6.90E−04 | 1.13 | 0.417 | 2034 |
| C04 | 21576609 | rs4377576 | C | 0.54 | 10634 | 3.25E−04 | 1.15 | 0.575 | 1700 | 5.32E−04 | 1.14 | 0.572 | 2034 |
| C04 | 21576609 | rs4377576 | C | 0.54 | 10634 | 3.25E−04 | 1.15 | 0.575 | 1700 | 5.32E−04 | 1.14 | 0.572 | 2034 |
| C12 | 65789635 | rs1465026 | G | 0.832 | 10632 | 3.27E−04 | 1.22 | 0.858 | 1700 | 8.82E−05 | 1.22 | 0.858 | 2034 |
| C18 | 2609280 | rs4271662 | C | 0.58 | 10594 | 3.34E−04 | 1.16 | 0.615 | 1695 | 9.97E−04 | 1.13 | 0.61 | 2026 |
| C01 | 150696580 | rs4845552 | G | 0.125 | 10588 | 3.40E−04 | 1.23 | 0.149 | 1698 | 2.40E−04 | 1.21 | 0.148 | 2030 |
| C15 | 24926452 | rs2110209 | A | 0.047 | 10635 | 3.42E−04 | 1.36 | 0.062 | 1700 | 9.53E−04 | 1.3 | 0.06 | 2034 |
| C03 | 170400106 | rs1430435 | G | 0.503 | 10634 | 3.44E−04 | 1.15 | 0.538 | 1700 | 2.59E−03 | 1.12 | 0.53 | 2034 |
| C06 | 72208904 | rs182106 | T | 0.523 | 10635 | 3.50E−04 | 1.15 | 0.558 | 1700 | 8.17E−04 | 1.13 | 0.554 | 2034 |
| C03 | 145877192 | rs10935515 | C | 0.583 | 10623 | 3.51E−04 | 1.15 | 0.618 | 1699 | 6.10E−04 | 1.14 | 0.614 | 2033 |
| C06 | 72149930 | rs9360453 | G | 0.711 | 10616 | 3.57E−04 | 1.17 | 0.742 | 1697 | 4.11E−04 | 1.16 | 0.74 | 2030 |
| C05 | 115427457 | rs17138668 | C | 0.947 | 10634 | 3.60E−04 | 1.41 | 0.962 | 1700 | 1.06E−03 | 1.34 | 0.96 | 2034 |
| CX | 91547326 | rs6615536 | G | 0.987 | 8272 | 3.62E−04 | 2.85 | 0.995 | 1103 | 1.19E−04 | 2.84 | 0.995 | 1316 |
| C03 | 194770024 | rs883279 | C | 0.905 | 10632 | 3.63E−04 | 1.29 | 0.925 | 1699 | 1.46E−04 | 1.29 | 0.925 | 2033 |
| C07 | 116680904 | rs2283054 | A | 0.069 | 10634 | 3.73E−04 | 1.3 | 0.087 | 1699 | 3.39E−04 | 1.27 | 0.086 | 2033 |
| C06 | 136340016 | rs9376173 | A | 0.629 | 10613 | 3.74E−04 | 1.16 | 0.663 | 1692 | 1.42E−03 | 1.13 | 0.657 | 2026 |
| C20 | 4152600 | rs1741318 | A | 0.447 | 10633 | 3.75E−04 | 1.15 | 0.482 | 1700 | 1.07E−03 | 1.13 | 0.477 | 2034 |
| C20 | 4152600 | rs1741318 | A | 0.447 | 10633 | 3.75E−04 | 1.15 | 0.482 | 1700 | 1.07E−03 | 1.13 | 0.477 | 2034 |
| C12 | 47206273 | rs12425518 | A | 0.043 | 10611 | 3.78E−04 | 1.37 | 0.058 | 1697 | 9.36E−03 | 1.25 | 0.053 | 2029 |
| C10 | 8196155 | rs541045 | C | 0.431 | 10501 | 3.79E−04 | 1.15 | 0.466 | 1659 | 1.79E−03 | 1.12 | 0.459 | 1992 |
| C01 | 20789662 | rs12741305 | C | 0.036 | 10635 | 3.85E−04 | 1.4 | 0.05 | 1698 | 1.09E−04 | 1.41 | 0.05 | 2031 |
| C07 | 116688223 | rs2237721 | C | 0.069 | 10634 | 3.85E−04 | 1.29 | 0.087 | 1700 | 3.50E−04 | 1.27 | 0.086 | 2034 |
| C08 | 5498753 | rs10503316 | C | 0.776 | 10635 | 3.87E−04 | 1.19 | 0.804 | 1700 | 2.83E−03 | 1.14 | 0.798 | 2034 |
| C05 | 119516035 | rs7721883 | T | 0.461 | 10597 | 3.89E−04 | 1.15 | 0.496 | 1698 | 7.26E−03 | 1.1 | 0.486 | 2032 |
| C02 | 135122883 | rs2139311 | C | 0.594 | 10622 | 3.90E−04 | 1.15 | 0.628 | 1699 | 3.72E−04 | 1.14 | 0.626 | 2033 |
| C01 | 113676295 | rs2476601 | A | 0.13 | 10633 | 3.93E−04 | 1.22 | 0.154 | 1700 | 2.39E−04 | 1.21 | 0.153 | 2034 |
| C18 | 53271813 | rs634285 | G | 0.913 | 10634 | 3.98E−04 | 1.3 | 0.931 | 1700 | 3.28E−03 | 1.22 | 0.927 | 2034 |
| C14 | 99695081 | rs17776453 | G | 0.951 | 10633 | 4.00E−04 | 1.43 | 0.966 | 1699 | 2.30E−04 | 1.41 | 0.965 | 2033 |
| C05 | 56242295 | rs252890 | A | 0.425 | 10632 | 4.02E−04 | 1.15 | 0.459 | 1699 | 5.18E−04 | 1.14 | 0.456 | 2033 |
| C01 | 40523163 | rs7520394 | T | 0.651 | 10619 | 4.13E−04 | 1.16 | 0.684 | 1698 | 6.36E−03 | 1.11 | 0.674 | 2032 |
| C22 | 44896799 | rs739164 | A | 0.894 | 10523 | 4.21E−04 | 1.28 | 0.915 | 1645 | 5.43E−04 | 1.25 | 0.913 | 1978 |
| C10 | 90674890 | rs2243548 | A | 0.509 | 10625 | 4.25E−04 | 1.15 | 0.544 | 1698 | 5.06E−03 | 1.11 | 0.534 | 2031 |
| C10 | 90674890 | rs2243548 | A | 0.509 | 10625 | 4.25E−04 | 1.15 | 0.544 | 1698 | 5.06E−03 | 1.11 | 0.534 | 2031 |
| CX | 108301033 | rs3788769 | C | 0.445 | 8210 | 4.25E−04 | 1.19 | 0.487 | 1095 | 1.26E−03 | 1.16 | 0.481 | 1307 |
| C22 | 34873996 | rs7364143 | T | 0.28 | 10592 | 4.25E−04 | 1.16 | 0.311 | 1696 | 6.90E−04 | 1.15 | 0.308 | 2030 |
| C03 | 191533619 | rs9864293 | C | 0.385 | 10632 | 4.43E−04 | 1.15 | 0.419 | 1700 | 9.88E−04 | 1.13 | 0.415 | 2033 |
| C07 | 102617539 | rs2270019 | G | 0.165 | 10614 | 4.44E−04 | 1.2 | 0.192 | 1697 | 2.59E−04 | 1.19 | 0.191 | 2031 |
| C20 | 15229479 | rs6034134 | G | 0.594 | 10634 | 4.48E−04 | 1.15 | 0.628 | 1700 | 1.86E−03 | 1.12 | 0.622 | 2034 |
| C05 | 153269681 | rs1461236 | T | 0.855 | 10614 | 4.49E−04 | 1.23 | 0.879 | 1689 | 4.26E−03 | 1.17 | 0.873 | 2023 |
| C11 | 92904904 | rs2658801 | A | 0.269 | 10634 | 4.49E−04 | 1.16 | 0.3 | 1700 | 1.76E−04 | 1.16 | 0.299 | 2034 |
| C17 | 10435400 | rs9903582 | C | 0.745 | 10634 | 4.53E−04 | 1.18 | 0.775 | 1700 | 1.92E−03 | 1.14 | 0.769 | 2034 |
| C15 | 84295353 | rs4887480 | G | 0.769 | 10629 | 4.55E−04 | 1.18 | 0.798 | 1700 | 3.82E−03 | 1.14 | 0.791 | 2034 |
| C17 | 17860913 | rs752579 | G | 0.388 | 10632 | 4.57E−04 | 1.15 | 0.421 | 1700 | 1.55E−03 | 1.12 | 0.416 | 2034 |
| C17 | 17860913 | rs752579 | G | 0.388 | 10632 | 4.57E−04 | 1.15 | 0.421 | 1700 | 1.55E−03 | 1.12 | 0.416 | 2034 |
| C14 | 92765567 | rs10498639 | A | 0.423 | 10633 | 4.57E−04 | 1.15 | 0.457 | 1700 | 1.35E−03 | 1.12 | 0.452 | 2034 |
| C20 | 284362 | rs723477 | T | 0.74 | 10613 | 4.57E−04 | 1.18 | 0.77 | 1694 | 2.16E−03 | 1.14 | 0.764 | 2027 |
| C05 | 10009076 | rs16883905 | T | 0.111 | 10635 | 4.59E−04 | 1.23 | 0.134 | 1700 | 7.81E−04 | 1.21 | 0.131 | 2034 |
| C06 | 106193116 | rs845851 | A | 0.766 | 10631 | 4.60E−04 | 1.18 | 0.794 | 1700 | 2.14E−03 | 1.14 | 0.789 | 2034 |
| C08 | 143178480 | rs9644545 | T | 0.476 | 10632 | 4.61E−04 | 1.15 | 0.511 | 1700 | 4.98E−03 | 1.11 | 0.502 | 2034 |
| C12 | 9180220 | rs2160424 | G | 0.557 | 10608 | 4.62E−04 | 1.15 | 0.591 | 1690 | 4.58E−03 | 1.11 | 0.583 | 2022 |
| C12 | 19360345 | rs10743315 | G | 0.878 | 10613 | 4.84E−04 | 1.25 | 0.9 | 1694 | 1.58E−03 | 1.2 | 0.896 | 2028 |
| C05 | 56298431 | rs2408654 | C | 0.273 | 10635 | 4.84E−04 | 1.16 | 0.304 | 1700 | 4.74E−04 | 1.15 | 0.302 | 2034 |
| C05 | 167473287 | rs7705176 | G | 0.781 | 10635 | 4.87E−04 | 1.19 | 0.809 | 1700 | 1.68E−03 | 1.15 | 0.805 | 2034 |
| C03 | 62396924 | rs1997366 | C | 0.792 | 10623 | 4.88E−04 | 1.19 | 0.82 | 1700 | 9.05E−04 | 1.17 | 0.817 | 2033 |
| C03 | 62396924 | rs1997366 | C | 0.792 | 10623 | 4.88E−04 | 1.19 | 0.82 | 1700 | 9.05E−04 | 1.17 | 0.817 | 2033 |
| C04 | 21494114 | rs6448072 | C | 0.655 | 10634 | 4.89E−04 | 1.16 | 0.688 | 1700 | 1.95E−04 | 1.16 | 0.687 | 2034 |
| C20 | 49593462 | rs913673 | C | 0.539 | 10624 | 4.90E−04 | 1.15 | 0.574 | 1700 | 1.89E−03 | 1.12 | 0.568 | 2034 |
| C10 | 119318254 | rs1557227 | C | 0.855 | 10635 | 4.91E−04 | 1.23 | 0.879 | 1700 | 4.19E−04 | 1.21 | 0.877 | 2034 |
| C05 | 158541049 | rs270661 | A | 0.205 | 10634 | 4.93E−04 | 1.18 | 0.233 | 1699 | 3.06E−03 | 1.14 | 0.227 | 2033 |
| C05 | 158541049 | rs270661 | A | 0.205 | 10634 | 4.93E−04 | 1.18 | 0.233 | 1699 | 3.06E−03 | 1.14 | 0.227 | 2033 |
| C06 | 72209498 | rs199623 | A | 0.523 | 10635 | 4.98E−04 | 1.15 | 0.557 | 1700 | 1.17E−03 | 1.13 | 0.552 | 2034 |
| C09 | 134953713 | rs2385891 | A | 0.544 | 10612 | 4.98E−04 | 1.15 | 0.578 | 1694 | 2.79E−03 | 1.12 | 0.571 | 2028 |
| C09 | 16321304 | rs9407756 | G | 0.67 | 10634 | 5.13E−04 | 1.16 | 0.702 | 1700 | 3.35E−03 | 1.12 | 0.695 | 2034 |
| C08 | 1315247 | rs7386449 | A | 0.68 | 10634 | 5.29E−04 | 1.16 | 0.711 | 1700 | 7.10E−04 | 1.14 | 0.708 | 2034 |
| C08 | 1315247 | rs7386449 | A | 0.68 | 10634 | 5.29E−04 | 1.16 | 0.711 | 1700 | 7.10E−04 | 1.14 | 0.708 | 2034 |
| C05 | 178690232 | rs3756566 | G | 0.401 | 10632 | 5.32E−04 | 1.15 | 0.434 | 1700 | 3.57E−04 | 1.14 | 0.433 | 2034 |
| C10 | 84919047 | rs11198993 | A | 0.688 | 10564 | 5.37E−04 | 1.16 | 0.719 | 1692 | 1.05E−05 | 1.19 | 0.725 | 2024 |
| C12 | 65801361 | rs1526839 | G | 0.831 | 10632 | 5.39E−04 | 1.21 | 0.856 | 1700 | 1.32E−04 | 1.21 | 0.856 | 2034 |
| C18 | 58503179 | rs12458349 | G | 0.061 | 10603 | 5.50E−04 | 1.3 | 0.078 | 1700 | 1.46E−04 | 1.31 | 0.078 | 2032 |
| C06 | 106204603 | rs4946680 | T | 0.632 | 10635 | 5.50E−04 | 1.15 | 0.665 | 1700 | 1.76E−03 | 1.13 | 0.66 | 2034 |
| C03 | 124473843 | rs9847700 | G | 0.852 | 10529 | 5.53E−04 | 1.23 | 0.876 | 1654 | 9.60E−04 | 1.2 | 0.873 | 1986 |
| C18 | 56405620 | rs603119 | T | 0.386 | 10606 | 5.54E−04 | 1.15 | 0.419 | 1699 | 9.25E−05 | 1.16 | 0.421 | 2032 |
| C06 | 113938753 | rs4945955 | T | 0.092 | 10614 | 5.55E−04 | 1.25 | 0.112 | 1697 | 5.18E−04 | 1.23 | 0.111 | 2031 |
| C05 | 26932085 | rs1382932 | C | 0.68 | 10635 | 5.60E−04 | 1.16 | 0.711 | 1700 | 1.13E−03 | 1.14 | 0.707 | 2034 |
| C05 | 76583322 | rs974280 | G | 0.357 | 10635 | 5.63E−04 | 1.15 | 0.39 | 1700 | 1.34E−03 | 1.13 | 0.385 | 2034 |
| C16 | 19044749 | rs7500550 | G | 0.774 | 10635 | 5.63E−04 | 1.18 | 0.802 | 1699 | 1.97E−03 | 1.15 | 0.798 | 2033 |
| C05 | 56284090 | rs1445998 | A | 0.4 | 10635 | 5.67E−04 | 1.15 | 0.433 | 1700 | 5.78E−04 | 1.14 | 0.43 | 2034 |
| C14 | 48189298 | rs4898630 | A | 0.876 | 10633 | 5.71E−04 | 1.24 | 0.898 | 1700 | 9.58E−04 | 1.21 | 0.895 | 2034 |
| C19 | 34828875 | rs4805463 | G | 0.906 | 10634 | 5.72E−04 | 1.28 | 0.925 | 1700 | 1.08E−03 | 1.24 | 0.923 | 2034 |
| C07 | 28352068 | rs1976489 | G | 0.249 | 10635 | 5.77E−04 | 1.17 | 0.279 | 1700 | 2.15E−03 | 1.14 | 0.274 | 2034 |
| C12 | 19263624 | rs9805106 | G | 0.817 | 10618 | 5.79E−04 | 1.2 | 0.843 | 1695 | 7.98E−04 | 1.18 | 0.84 | 2029 |
| C17 | 17915667 | rs11868035 | A | 0.296 | 10623 | 5.79E−04 | 1.16 | 0.327 | 1699 | 5.61E−03 | 1.12 | 0.319 | 2033 |
| C17 | 17915667 | rs11868035 | A | 0.296 | 10623 | 5.79E−04 | 1.16 | 0.327 | 1699 | 5.61E−03 | 1.12 | 0.319 | 2033 |
| C01 | 209613176 | rs792446 | G | 0.611 | 10634 | 5.80E−04 | 1.15 | 0.644 | 1700 | 1.30E−02 | 1.1 | 0.633 | 2033 |
| C13 | 109862872 | rs1163852 | C | 0.714 | 10635 | 5.83E−04 | 1.17 | 0.744 | 1700 | 2.89E−04 | 1.16 | 0.743 | 2034 |
| C03 | 22619885 | rs1870480 | G | 0.38 | 10635 | 5.85E−04 | 1.15 | 0.413 | 1700 | 3.12E−04 | 1.14 | 0.412 | 2034 |
| C15 | 97226804 | rs2684790 | T | 0.146 | 10634 | 5.87E−04 | 1.2 | 0.17 | 1700 | 7.79E−04 | 1.18 | 0.168 | 2034 |
| C14 | 44542892 | rs1742655 | C | 0.884 | 10635 | 5.91E−04 | 1.25 | 0.905 | 1700 | 3.42E−03 | 1.19 | 0.901 | 2034 |
| C12 | 114213023 | rs1920947 | C | 0.812 | 10635 | 5.92E−04 | 1.2 | 0.838 | 1700 | 7.20E−03 | 1.14 | 0.831 | 2034 |
| C01 | 30387821 | rs12029866 | G | 0.771 | 10634 | 5.93E−04 | 1.18 | 0.799 | 1700 | 1.33E−04 | 1.19 | 0.8 | 2034 |
| C11 | 91095458 | rs2568712 | T | 0.385 | 10599 | 5.98E−04 | 1.15 | 0.418 | 1699 | 1.68E−03 | 1.12 | 0.413 | 2031 |
| C01 | 168129978 | rs11582943 | A | 0.94 | 10619 | 5.99E−04 | 1.36 | 0.955 | 1693 | 6.87E−04 | 1.33 | 0.954 | 2027 |
| C01 | 168129978 | rs11582943 | A | 0.94 | 10619 | 5.99E−04 | 1.36 | 0.955 | 1693 | 6.87E−04 | 1.33 | 0.954 | 2027 |
| C02 | 49219942 | rs3788982 | G | 0.871 | 10635 | 6.02E−04 | 1.24 | 0.893 | 1700 | 8.11E−05 | 1.25 | 0.894 | 2034 |
| C10 | 131670000 | rs639079 | G | 0.187 | 10389 | 6.03E−04 | 1.18 | 0.214 | 1664 | 7.05E−04 | 1.17 | 0.212 | 1992 |
| C01 | 76636957 | rs1566250 | G | 0.702 | 10634 | 6.13E−04 | 1.16 | 0.732 | 1700 | 1.98E−04 | 1.16 | 0.733 | 2034 |
| C12 | 87964674 | rs1401875 | T | 0.057 | 10626 | 6.13E−04 | 1.31 | 0.073 | 1697 | 1.83E−03 | 1.26 | 0.07 | 2031 |
| C12 | 87964674 | rs1401875 | T | 0.057 | 10626 | 6.13E−04 | 1.31 | 0.073 | 1697 | 1.83E−03 | 1.26 | 0.07 | 2031 |
| C05 | 158753477 | rs953861 | G | 0.16 | 10632 | 6.14E−04 | 1.19 | 0.185 | 1700 | 1.51E−02 | 1.13 | 0.176 | 2034 |
| C01 | 82599958 | rs12032817 | G | 0.371 | 10635 | 6.15E−04 | 1.15 | 0.404 | 1700 | 6.29E−03 | 1.11 | 0.396 | 2034 |
| C03 | 78544480 | rs2055707 | A | 0.418 | 10603 | 6.20E−04 | 1.15 | 0.451 | 1699 | 2.42E−03 | 1.12 | 0.445 | 2033 |
| C19 | 4231186 | rs10419363 | T | 0.299 | 10610 | 6.22E−04 | 1.16 | 0.331 | 1695 | 8.39E−04 | 1.14 | 0.328 | 2028 |
| C02 | 235330231 | rs12617590 | C | 0.199 | 10624 | 6.29E−04 | 1.18 | 0.227 | 1700 | 2.26E−03 | 1.15 | 0.222 | 2034 |
| C04 | 87398087 | rs2589506 | G | 0.451 | 10632 | 6.31E−04 | 1.14 | 0.485 | 1700 | 1.15E−04 | 1.15 | 0.486 | 2034 |
| C10 | 87792858 | rs3814614 | T | 0.501 | 10628 | 6.35E−04 | 1.14 | 0.535 | 1700 | 1.05E−03 | 1.13 | 0.531 | 2034 |
| C04 | 97290133 | rs10516987 | G | 0.797 | 10598 | 6.35E−04 | 1.19 | 0.824 | 1691 | 1.55E−03 | 1.16 | 0.82 | 2023 |
| C13 | 101108783 | rs1375719 | C | 0.499 | 10634 | 6.36E−04 | 1.14 | 0.532 | 1700 | 1.03E−02 | 1.1 | 0.522 | 2034 |
| C17 | 18231090 | rs854793 | A | 0.202 | 10631 | 6.36E−04 | 1.18 | 0.229 | 1700 | 1.59E−03 | 1.15 | 0.225 | 2034 |
| C15 | 76722261 | rs11072793 | G | 0.227 | 10613 | 6.38E−04 | 1.17 | 0.255 | 1695 | 1.52E−03 | 1.14 | 0.251 | 2029 |
| C09 | 8437732 | rs2890795 | T | 0.577 | 10630 | 6.38E−04 | 1.15 | 0.61 | 1700 | 1.20E−03 | 1.13 | 0.606 | 2034 |
| C07 | 67296404 | rs4527771 | A | 0.406 | 10572 | 6.43E−04 | 1.15 | 0.439 | 1694 | 8.45E−03 | 1.1 | 0.43 | 2027 |
| C13 | 66843127 | rs17070671 | T | 0.833 | 10633 | 6.44E−04 | 1.21 | 0.857 | 1700 | 9.00E−03 | 1.14 | 0.85 | 2034 |
| C16 | 73542120 | rs1038864 | C | 0.183 | 10634 | 6.45E−04 | 1.18 | 0.209 | 1700 | 4.45E−03 | 1.14 | 0.203 | 2034 |
| C06 | 25536933 | rs722086 | T | 0.856 | 10166 | 6.47E−04 | 1.22 | 0.879 | 1687 | 7.47E−03 | 1.16 | 0.873 | 2015 |
| C08 | 70811871 | rs4738034 | C | 0.701 | 10635 | 6.49E−04 | 1.16 | 0.731 | 1700 | 9.61E−04 | 1.14 | 0.728 | 2034 |
| C18 | 55095560 | rs657773 | C | 0.551 | 10634 | 6.57E−04 | 1.15 | 0.585 | 1700 | 1.04E−03 | 1.13 | 0.581 | 2034 |
| C02 | 134844429 | rs2321299 | C | 0.568 | 10633 | 6.58E−04 | 1.15 | 0.601 | 1700 | 3.50E−03 | 1.11 | 0.595 | 2033 |
| C06 | 149543409 | rs6570956 | T | 0.734 | 10635 | 6.61E−04 | 1.17 | 0.764 | 1700 | 6.82E−03 | 1.12 | 0.756 | 2034 |
| C12 | 116667030 | rs4767629 | T | 0.139 | 10634 | 6.61E−04 | 1.2 | 0.163 | 1700 | 5.33E−03 | 1.15 | 0.157 | 2034 |
| C01 | 80891035 | rs1030414 | G | 0.901 | 10635 | 6.73E−04 | 1.27 | 0.92 | 1700 | 3.45E−04 | 1.26 | 0.92 | 2033 |
| C06 | 69176005 | rs1565487 | A | 0.856 | 10630 | 6.74E−04 | 1.22 | 0.879 | 1700 | 2.07E−04 | 1.22 | 0.879 | 2034 |
| C10 | 123913719 | rs2239586 | T | 0.118 | 10630 | 6.75E−04 | 1.22 | 0.14 | 1700 | 2.95E−03 | 1.18 | 0.136 | 2034 |
| C11 | 122290366 | rs4936757 | C | 0.565 | 10633 | 6.76E−04 | 1.15 | 0.598 | 1700 | 3.91E−03 | 1.11 | 0.591 | 2034 |
| C12 | 22090132 | rs2955503 | A | 0.654 | 10629 | 6.82E−04 | 1.15 | 0.686 | 1700 | 2.41E−04 | 1.15 | 0.686 | 2033 |
| C17 | 52089014 | rs11656018 | C | 0.703 | 10600 | 6.84E−04 | 1.16 | 0.733 | 1699 | 8.06E−04 | 1.15 | 0.731 | 2032 |
| C06 | 159976575 | rs2758317 | C | 0.427 | 10572 | 6.86E−04 | 1.14 | 0.46 | 1690 | 6.67E−03 | 1.11 | 0.452 | 2019 |
| C18 | 43266738 | rs17734724 | G | 0.271 | 10635 | 6.87E−04 | 1.16 | 0.301 | 1700 | 3.27E−03 | 1.13 | 0.295 | 2034 |
| C02 | 19979982 | rs12467812 | G | 0.331 | 10634 | 6.88E−04 | 1.15 | 0.363 | 1700 | 2.30E−04 | 1.15 | 0.363 | 2034 |
| C15 | 51085506 | rs1156234 | G | 0.791 | 10633 | 6.91E−04 | 1.18 | 0.818 | 1700 | 5.59E−04 | 1.17 | 0.816 | 2034 |
| C10 | 123156230 | rs2936875 | T | 0.782 | 10629 | 6.92E−04 | 1.18 | 0.809 | 1699 | 4.42E−03 | 1.14 | 0.803 | 2033 |
| C19 | 58638423 | rs11668495 | C | 0.944 | 10470 | 7.08E−04 | 1.38 | 0.959 | 1668 | 1.43E−03 | 1.32 | 0.957 | 1995 |
| C07 | 136198861 | rs11761344 | T | 0.116 | 10635 | 7.12E−04 | 1.22 | 0.138 | 1699 | 8.84E−04 | 1.2 | 0.136 | 2033 |
| C10 | 84739213 | rs4617529 | G | 0.852 | 10635 | 7.15E−04 | 1.22 | 0.875 | 1700 | 6.68E−04 | 1.2 | 0.873 | 2034 |
| C08 | 139196618 | rs4909761 | T | 0.262 | 10634 | 7.20E−04 | 1.16 | 0.291 | 1700 | 2.65E−03 | 1.13 | 0.286 | 2034 |
| C05 | 94602573 | rs6556861 | G | 0.567 | 10634 | 7.24E−04 | 1.14 | 0.6 | 1700 | 2.99E−03 | 1.12 | 0.594 | 2034 |
| C09 | 132606652 | rs1930788 | C | 0.247 | 10635 | 7.27E−04 | 1.16 | 0.276 | 1700 | 4.10E−03 | 1.13 | 0.27 | 2034 |
| C05 | 157926810 | rs2964513 | A | 0.689 | 10559 | 7.29E−04 | 1.16 | 0.72 | 1698 | 3.01E−03 | 1.13 | 0.714 | 2030 |
| C08 | 5508780 | rs6991017 | T | 0.794 | 10607 | 7.30E−04 | 1.19 | 0.82 | 1698 | 5.36E−03 | 1.14 | 0.814 | 2032 |
| C02 | 135133800 | rs12473666 | T | 0.137 | 10629 | 7.31E−04 | 1.2 | 0.16 | 1700 | 2.94E−03 | 1.17 | 0.156 | 2034 |
| C17 | 18149041 | rs2955355 | C | 0.287 | 10635 | 7.35E−04 | 1.16 | 0.317 | 1700 | 3.19E−03 | 1.12 | 0.311 | 2034 |
| C11 | 44349237 | rs10838271 | C | 0.21 | 10635 | 7.42E−04 | 1.17 | 0.238 | 1700 | 3.32E−03 | 1.14 | 0.233 | 2034 |
| C01 | 238617451 | rs1341449 | A | 0.545 | 10559 | 7.44E−04 | 1.14 | 0.579 | 1681 | 1.15E−03 | 1.13 | 0.575 | 2014 |
| C15 | 34765965 | rs1901725 | T | 0.125 | 10635 | 7.47E−04 | 1.21 | 0.148 | 1700 | 3.63E−03 | 1.17 | 0.143 | 2034 |
| C10 | 87786571 | rs4933391 | A | 0.386 | 10634 | 7.50E−04 | 1.14 | 0.418 | 1700 | 1.30E−03 | 1.13 | 0.414 | 2034 |
| C01 | 80911800 | rs7531507 | G | 0.921 | 10634 | 7.52E−04 | 1.3 | 0.938 | 1700 | 3.18E−04 | 1.3 | 0.938 | 2034 |
| C14 | 57289229 | rs2069334 | A | 0.184 | 10635 | 7.54E−04 | 1.18 | 0.211 | 1700 | 6.14E−03 | 1.13 | 0.204 | 2034 |
| C02 | 17299060 | rs2342551 | T | 0.713 | 10635 | 7.54E−04 | 1.16 | 0.743 | 1700 | 1.23E−03 | 1.14 | 0.74 | 2034 |
| C02 | 128492107 | rs2244871 | C | 0.41 | 10632 | 7.62E−04 | 1.14 | 0.442 | 1700 | 7.38E−04 | 1.13 | 0.44 | 2034 |
| C16 | 75061517 | rs1862737 | C | 0.481 | 10635 | 7.67E−04 | 1.14 | 0.514 | 1700 | 1.75E−03 | 1.12 | 0.51 | 2034 |
| C04 | 129868334 | rs7690289 | T | 0.63 | 10596 | 7.70E−04 | 1.15 | 0.661 | 1697 | 7.24E−04 | 1.14 | 0.659 | 2031 |
| C04 | 129868334 | rs7690289 | T | 0.63 | 10596 | 7.70E−04 | 1.15 | 0.661 | 1697 | 7.24E−04 | 1.14 | 0.659 | 2031 |
| C18 | 73086657 | rs2156643 | G | 0.41 | 10631 | 7.70E−04 | 1.14 | 0.443 | 1700 | 1.49E−03 | 1.12 | 0.439 | 2034 |
| C11 | 7440298 | rs11041390 | A | 0.729 | 10635 | 7.70E−04 | 1.16 | 0.758 | 1700 | 8.75E−03 | 1.12 | 0.75 | 2034 |
| C15 | 34765482 | rs7183966 | G | 0.125 | 10632 | 7.75E−04 | 1.21 | 0.148 | 1698 | 3.73E−03 | 1.17 | 0.143 | 2032 |
| C09 | 28114180 | rs1452336 | A | 0.495 | 10608 | 7.76E−04 | 1.14 | 0.529 | 1695 | 1.89E−04 | 1.15 | 0.53 | 2029 |
| C05 | 102480237 | rs34374 | A | 0.671 | 10635 | 7.80E−04 | 1.15 | 0.702 | 1699 | 5.86E−03 | 1.11 | 0.695 | 2033 |
| C05 | 82104191 | rs6452479 | C | 0.934 | 10595 | 7.88E−04 | 1.33 | 0.95 | 1695 | 6.46E−04 | 1.31 | 0.949 | 2028 |
| C18 | 73085584 | rs2850889 | C | 0.41 | 10634 | 7.91E−04 | 1.14 | 0.443 | 1700 | 1.53E−03 | 1.12 | 0.439 | 2034 |
| C11 | 20014579 | rs920675 | A | 0.358 | 10635 | 7.95E−04 | 1.15 | 0.39 | 1700 | 5.37E−04 | 1.14 | 0.389 | 2034 |
| C03 | 39292564 | rs2853705 | T | 0.209 | 10634 | 7.97E−04 | 1.17 | 0.236 | 1700 | 2.79E−03 | 1.14 | 0.231 | 2034 |
| C18 | 73086818 | rs2850892 | C | 0.41 | 10631 | 7.98E−04 | 1.14 | 0.443 | 1700 | 1.55E−03 | 1.12 | 0.439 | 2034 |
| C04 | 178149338 | rs309770 | G | 0.889 | 10635 | 8.01E−04 | 1.25 | 0.909 | 1700 | 5.03E−04 | 1.24 | 0.908 | 2034 |
| C11 | 7444767 | rs7113222 | G | 0.739 | 10632 | 8.03E−04 | 1.17 | 0.768 | 1700 | 1.29E−02 | 1.11 | 0.759 | 2034 |
| C03 | 6735499 | rs7638636 | A | 0.494 | 10231 | 8.03E−04 | 1.14 | 0.528 | 1648 | 5.59E−04 | 1.14 | 0.526 | 1972 |
| C02 | 42489923 | rs10202624 | A | 0.094 | 10634 | 8.15E−04 | 1.24 | 0.114 | 1700 | 1.79E−04 | 1.25 | 0.114 | 2034 |
| C06 | 20707867 | rs2294809 | G | 0.799 | 10626 | 8.19E−04 | 1.19 | 0.825 | 1699 | 3.18E−03 | 1.15 | 0.82 | 2033 |
| C02 | 118487620 | rs2245113 | A | 0.174 | 10632 | 8.20E−04 | 1.18 | 0.199 | 1700 | 1.79E−02 | 1.12 | 0.191 | 2034 |
| C19 | 17024661 | rs2305758 | G | 0.684 | 10633 | 8.21E−04 | 1.16 | 0.714 | 1700 | 4.38E−04 | 1.15 | 0.714 | 2034 |
| C19 | 17024661 | rs2305758 | G | 0.684 | 10633 | 8.21E−04 | 1.16 | 0.714 | 1700 | 4.38E−04 | 1.15 | 0.714 | 2034 |
| C01 | 63908878 | rs1747924 | T | 0.808 | 10622 | 8.23E−04 | 1.19 | 0.834 | 1700 | 9.80E−03 | 1.13 | 0.827 | 2034 |
| C22 | 34883676 | rs5995259 | A | 0.337 | 10635 | 8.26E−04 | 1.15 | 0.369 | 1700 | 2.83E−03 | 1.12 | 0.363 | 2034 |
| C02 | 45292999 | rs908571 | C | 0.291 | 10635 | 8.27E−04 | 1.15 | 0.321 | 1699 | 1.62E−03 | 1.13 | 0.318 | 2033 |
| C20 | 33195666 | rs734484 | C | 0.709 | 10635 | 8.29E−04 | 1.16 | 0.738 | 1699 | 2.20E−03 | 1.13 | 0.734 | 2033 |
| C16 | 2009168 | rs8057913 | C | 0.264 | 10634 | 8.39E−04 | 1.16 | 0.293 | 1700 | 1.13E−03 | 1.14 | 0.29 | 2034 |
| C03 | 99699602 | rs3796139 | G | 0.145 | 10476 | 8.40E−04 | 1.2 | 0.169 | 1685 | 4.25E−03 | 1.16 | 0.164 | 2011 |
| C02 | 135194839 | rs1257208 | A | 0.138 | 10633 | 8.41E−04 | 1.2 | 0.161 | 1700 | 1.98E−03 | 1.17 | 0.158 | 2034 |
| C20 | 5928108 | rs236110 | G | 0.87 | 10635 | 8.43E−04 | 1.23 | 0.892 | 1698 | 9.17E−04 | 1.21 | 0.89 | 2031 |
| C05 | 3644654 | rs710998 | T | 0.899 | 10630 | 8.55E−04 | 1.26 | 0.918 | 1699 | 1.20E−03 | 1.23 | 0.917 | 2033 |
| C02 | 49398605 | rs6743414 | G | 0.847 | 10634 | 8.62E−04 | 1.21 | 0.87 | 1700 | 1.51E−04 | 1.22 | 0.871 | 2034 |
| C13 | 38516680 | rs9315759 | C | 0.13 | 10635 | 8.63E−04 | 1.21 | 0.152 | 1700 | 3.14E−03 | 1.17 | 0.148 | 2034 |
| C01 | 184657396 | rs4651384 | G | 0.687 | 10634 | 8.72E−04 | 1.15 | 0.717 | 1700 | 4.41E−03 | 1.12 | 0.711 | 2034 |
| C19 | 17031885 | rs12459084 | T | 0.351 | 10633 | 8.76E−04 | 1.15 | 0.382 | 1700 | 3.24E−03 | 1.12 | 0.376 | 2034 |
| C17 | 48847103 | rs4794128 | A | 0.272 | 10634 | 8.77E−04 | 1.16 | 0.302 | 1700 | 1.05E−03 | 1.14 | 0.299 | 2034 |
| C08 | 32544721 | rs12547858 | T | 0.195 | 10624 | 8.80E−04 | 1.17 | 0.221 | 1700 | 7.59E−04 | 1.16 | 0.22 | 2034 |
| C09 | 16318137 | rs4961695 | C | 0.803 | 10614 | 8.81E−04 | 1.19 | 0.829 | 1697 | 1.23E−03 | 1.17 | 0.826 | 2029 |
| C02 | 170296713 | rs2268365 | G | 0.125 | 10635 | 8.83E−04 | 1.21 | 0.147 | 1698 | 2.14E−03 | 1.18 | 0.144 | 2032 |
| C02 | 170296713 | rs2268365 | G | 0.125 | 10635 | 8.83E−04 | 1.21 | 0.147 | 1698 | 2.14E−03 | 1.18 | 0.144 | 2032 |
| C07 | 17752626 | rs4470902 | C | 0.417 | 10634 | 8.84E−04 | 1.14 | 0.45 | 1700 | 8.58E−04 | 1.13 | 0.447 | 2034 |
| C14 | 57303694 | rs17095237 | G | 0.16 | 10634 | 8.85E−04 | 1.19 | 0.185 | 1700 | 6.54E−03 | 1.14 | 0.179 | 2034 |
| C05 | 38969280 | rs357291 | C | 0.395 | 10579 | 8.89E−04 | 1.14 | 0.428 | 1688 | 6.20E−03 | 1.11 | 0.42 | 2020 |
| C06 | 30109076 | rs166327 | A | 0.601 | 10634 | 8.91E−04 | 1.14 | 0.633 | 1700 | 4.98E−03 | 1.11 | 0.626 | 2034 |
| C03 | 125374402 | rs6438839 | A | 0.077 | 10632 | 8.92E−04 | 1.26 | 0.095 | 1700 | 4.40E−04 | 1.25 | 0.095 | 2034 |
| C05 | 153231957 | rs1870739 | C | 0.855 | 10629 | 8.92E−04 | 1.21 | 0.877 | 1697 | 6.74E−03 | 1.16 | 0.872 | 2031 |
| C01 | 40507124 | rs661221 | G | 0.803 | 10614 | 8.92E−04 | 1.19 | 0.828 | 1697 | 8.83E−03 | 1.13 | 0.821 | 2030 |
| C05 | 50944695 | rs4865673 | G | 0.643 | 10635 | 8.94E−04 | 1.15 | 0.674 | 1700 | 1.99E−03 | 1.13 | 0.67 | 2034 |
| C03 | 186444329 | rs6803944 | C | 0.841 | 10635 | 8.95E−04 | 1.21 | 0.865 | 1700 | 7.54E−04 | 1.19 | 0.863 | 2034 |
| C05 | 102439618 | rs26434 | T | 0.704 | 10617 | 8.95E−04 | 1.16 | 0.734 | 1697 | 5.12E−03 | 1.12 | 0.727 | 2031 |
| C10 | 29327574 | rs788053 | T | 0.747 | 10635 | 9.01E−04 | 1.17 | 0.775 | 1700 | 1.80E−03 | 1.14 | 0.771 | 2034 |
| C12 | 101671864 | rs12311520 | C | 0.867 | 10624 | 9.12E−04 | 1.22 | 0.889 | 1700 | 5.86E−03 | 1.17 | 0.884 | 2033 |
| C01 | 29565842 | rs1932397 | G | 0.821 | 10635 | 9.14E−04 | 1.19 | 0.845 | 1700 | 2.07E−03 | 1.16 | 0.842 | 2034 |
| C04 | 10653042 | rs959233 | G | 0.445 | 10622 | 9.20E−04 | 1.14 | 0.477 | 1700 | 1.23E−03 | 1.13 | 0.474 | 2033 |
| C08 | 144018676 | rs5297 | C | 0.09 | 10632 | 9.22E−04 | 1.24 | 0.109 | 1700 | 4.01E−03 | 1.19 | 0.105 | 2034 |
| C08 | 143110958 | rs747551 | T | 0.436 | 10633 | 9.27E−04 | 1.14 | 0.469 | 1700 | 1.39E−03 | 1.12 | 0.465 | 2034 |
| C18 | 55790697 | rs8082768 | T | 0.18 | 10621 | 9.28E−04 | 1.18 | 0.206 | 1700 | 1.97E−04 | 1.19 | 0.207 | 2034 |
| C12 | 111537264 | rs1015249 | C | 0.689 | 10635 | 9.36E−04 | 1.15 | 0.719 | 1700 | 1.59E−03 | 1.13 | 0.715 | 2034 |
| C08 | 59026980 | rs7010934 | C | 0.842 | 10632 | 9.45E−04 | 1.2 | 0.865 | 1700 | 2.40E−03 | 1.17 | 0.862 | 2034 |
| C01 | 78361370 | rs1412414 | G | 0.621 | 10634 | 9.53E−04 | 1.15 | 0.652 | 1699 | 5.85E−03 | 1.11 | 0.645 | 2033 |
| C06 | 165543591 | rs1744503 | T | 0.478 | 10631 | 9.57E−04 | 1.14 | 0.511 | 1700 | 2.74E−04 | 1.14 | 0.512 | 2033 |
| C01 | 76521831 | rs10518531 | C | 0.842 | 10561 | 9.57E−04 | 1.21 | 0.865 | 1668 | 3.57E−04 | 1.21 | 0.865 | 1998 |
| C12 | 119365986 | rs719450 | C | 0.918 | 10635 | 9.57E−04 | 1.29 | 0.935 | 1700 | 2.69E−04 | 1.3 | 0.935 | 2034 |
| C12 | 119365986 | rs719450 | C | 0.918 | 10635 | 9.57E−04 | 1.29 | 0.935 | 1700 | 2.69E−04 | 1.3 | 0.935 | 2034 |
| C03 | 175246538 | rs1421420 | A | 0.725 | 10633 | 9.62E−04 | 1.16 | 0.754 | 1700 | 2.21E−02 | 1.1 | 0.743 | 2034 |
| C06 | 29542163 | rs2107191 | G | 0.439 | 10634 | 9.63E−04 | 1.14 | 0.471 | 1700 | 4.51E−03 | 1.11 | 0.465 | 2034 |
| C06 | 29542163 | rs2107191 | G | 0.439 | 10634 | 9.63E−04 | 1.14 | 0.471 | 1700 | 4.51E−03 | 1.11 | 0.465 | 2034 |
| C05 | 76806011 | rs3816609 | A | 0.356 | 10632 | 9.64E−04 | 1.14 | 0.387 | 1700 | 2.31E−03 | 1.12 | 0.382 | 2034 |
| C18 | 56570151 | rs2332026 | A | 0.499 | 10634 | 9.72E−04 | 1.14 | 0.532 | 1700 | 1.79E−03 | 1.12 | 0.528 | 2034 |
| C17 | 68940662 | rs312750 | C | 0.547 | 10633 | 9.72E−04 | 1.14 | 0.579 | 1700 | 7.18E−03 | 1.1 | 0.572 | 2034 |
| C03 | 72792674 | rs2208 | A | 0.265 | 10634 | 9.76E−04 | 1.16 | 0.294 | 1700 | 4.77E−03 | 1.12 | 0.288 | 2034 |
| C03 | 72733647 | rs7632726 | T | 0.307 | 10635 | 9.77E−04 | 1.15 | 0.337 | 1700 | 3.48E−03 | 1.12 | 0.332 | 2034 |
| C03 | 145911459 | rs4401402 | T | 0.583 | 10633 | 9.79E−04 | 1.14 | 0.615 | 1700 | 1.80E−03 | 1.12 | 0.611 | 2034 |
| C04 | 30752184 | rs4132132 | G | 0.439 | 10618 | 9.80E−04 | 1.14 | 0.471 | 1696 | 7.47E−04 | 1.13 | 0.47 | 2030 |
| C01 | 88967295 | rs10801705 | A | 0.456 | 10635 | 9.80E−04 | 1.14 | 0.489 | 1699 | 8.76E−04 | 1.13 | 0.487 | 2033 |
| C10 | 23151213 | rs2935689 | C | 0.837 | 10635 | 9.81E−04 | 1.2 | 0.86 | 1700 | 6.25E−03 | 1.15 | 0.855 | 2034 |
| C18 | 39875310 | rs1015735 | C | 0.262 | 10605 | 9.82E−04 | 1.16 | 0.291 | 1695 | 2.57E−03 | 1.13 | 0.286 | 2029 |
| C04 | 82259686 | rs10026644 | G | 0.926 | 10619 | 9.83E−04 | 1.3 | 0.942 | 1697 | 4.26E−03 | 1.23 | 0.939 | 2028 |
| C20 | 4111436 | rs1741285 | G | 0.56 | 10634 | 9.88E−04 | 1.14 | 0.592 | 1698 | 1.72E−03 | 1.12 | 0.588 | 2032 |
| C20 | 4111436 | rs1741285 | G | 0.56 | 10634 | 9.88E−04 | 1.14 | 0.592 | 1698 | 1.72E−03 | 1.12 | 0.588 | 2032 |
| C14 | 47963078 | rs8007161 | T | 0.884 | 10602 | 9.90E−04 | 1.24 | 0.904 | 1698 | 1.63E−03 | 1.21 | 0.901 | 2030 |
| C14 | 57288897 | rs392030 | G | 0.635 | 10635 | 9.98E−04 | 1.15 | 0.666 | 1700 | 2.27E−03 | 1.12 | 0.661 | 2034 |
| C01 | 19432522 | rs7523192 | T | 0.809 | 10634 | 1.01E−03 | 1.19 | 0.834 | 1700 | 2.17E−02 | 1.12 | 0.826 | 2034 |
| C08 | 144284828 | rs4977114 | C | 0.026 | 10635 | 1.01E−03 | 1.44 | 0.037 | 1700 | 7.98E−04 | 1.42 | 0.036 | 2034 |
| CX | 4923358 | rs1919026 | C | 0.189 | 8271 | 1.02E−03 | 1.22 | 0.221 | 1103 | 4.66E−03 | 1.17 | 0.214 | 1316 |
| C01 | 18207864 | rs2946534 | T | 0.572 | 10631 | 1.03E−03 | 1.14 | 0.604 | 1700 | 7.54E−04 | 1.13 | 0.602 | 2034 |
| C09 | 28116482 | rs1452337 | G | 0.798 | 10568 | 1.03E−03 | 1.18 | 0.824 | 1687 | 1.01E−03 | 1.17 | 0.822 | 2021 |
| C19 | 17039119 | rs7254154 | C | 0.351 | 10617 | 1.03E−03 | 1.14 | 0.383 | 1695 | 3.90E−03 | 1.12 | 0.377 | 2029 |
| C19 | 17039119 | rs7254154 | C | 0.351 | 10617 | 1.03E−03 | 1.14 | 0.383 | 1695 | 3.90E−03 | 1.12 | 0.377 | 2029 |
| C08 | 73727022 | rs1837544 | G | 0.24 | 10634 | 1.03E−03 | 1.16 | 0.268 | 1700 | 2.02E−03 | 1.14 | 0.264 | 2034 |
| C02 | 183500703 | rs1946815 | G | 0.216 | 10634 | 1.03E−03 | 1.17 | 0.243 | 1700 | 2.25E−03 | 1.14 | 0.239 | 2034 |
| C07 | 102702234 | rs17152161 | C | 0.159 | 10633 | 1.03E−03 | 1.19 | 0.183 | 1700 | 5.79E−03 | 1.14 | 0.177 | 2034 |
| C02 | 216388367 | rs13417729 | C | 0.713 | 10635 | 1.04E−03 | 1.16 | 0.742 | 1700 | 1.20E−03 | 1.14 | 0.74 | 2034 |
| C20 | 13933789 | rs6079235 | C | 0.735 | 10635 | 1.04E−03 | 1.16 | 0.763 | 1700 | 1.05E−02 | 1.11 | 0.755 | 2034 |
| C04 | 8425936 | rs3756193 | G | 0.46 | 10623 | 1.05E−03 | 1.14 | 0.492 | 1699 | 2.26E−03 | 1.12 | 0.488 | 2033 |
| C05 | 76635083 | rs4704400 | T | 0.496 | 10628 | 1.06E−03 | 1.14 | 0.528 | 1699 | 3.23E−03 | 1.11 | 0.523 | 2033 |
| C17 | 17897321 | rs11649804 | A | 0.292 | 10506 | 1.06E−03 | 1.15 | 0.322 | 1650 | 7.96E−03 | 1.11 | 0.315 | 1979 |
| C17 | 17897321 | rs11649804 | A | 0.292 | 10506 | 1.06E−03 | 1.15 | 0.322 | 1650 | 7.96E−03 | 1.11 | 0.315 | 1979 |
| C14 | 64588026 | rs17754686 | A | 0.306 | 10606 | 1.06E−03 | 1.15 | 0.336 | 1698 | 1.49E−03 | 1.13 | 0.333 | 2032 |
| C16 | 2013121 | rs8045288 | T | 0.266 | 10618 | 1.06E−03 | 1.15 | 0.295 | 1700 | 1.23E−03 | 1.14 | 0.293 | 2034 |
| C04 | 139047812 | rs10028163 | G | 0.202 | 10634 | 1.06E−03 | 1.17 | 0.229 | 1700 | 4.06E−04 | 1.17 | 0.229 | 2034 |
| C14 | 98131730 | rs7158073 | T | 0.429 | 10592 | 1.06E−03 | 1.14 | 0.461 | 1696 | 2.61E−03 | 1.12 | 0.457 | 2029 |
| C07 | 42943442 | rs3757564 | C | 0.069 | 10624 | 1.07E−03 | 1.27 | 0.085 | 1696 | 1.02E−03 | 1.25 | 0.084 | 2029 |
| C11 | 110045689 | rs11604632 | A | 0.173 | 10632 | 1.07E−03 | 1.18 | 0.198 | 1700 | 4.90E−04 | 1.18 | 0.197 | 2032 |
| C17 | 17862368 | rs4925109 | A | 0.317 | 10634 | 1.07E−03 | 1.15 | 0.348 | 1700 | 3.75E−03 | 1.12 | 0.342 | 2034 |
| C17 | 17862368 | rs4925109 | A | 0.317 | 10634 | 1.07E−03 | 1.15 | 0.348 | 1700 | 3.75E−03 | 1.12 | 0.342 | 2034 |
| C12 | 96888528 | rs7135604 | A | 0.652 | 10635 | 1.07E−03 | 1.15 | 0.682 | 1699 | 2.11E−02 | 1.09 | 0.672 | 2033 |
| C11 | 97095503 | rs1039953 | C | 0.68 | 10635 | 1.08E−03 | 1.15 | 0.71 | 1700 | 1.67E−03 | 1.13 | 0.706 | 2034 |
| C04 | 163985671 | rs9991428 | G | 0.96 | 10613 | 1.08E−03 | 1.44 | 0.972 | 1699 | 3.03E−03 | 1.35 | 0.97 | 2033 |
| C01 | 148995481 | rs9826 | G | 0.326 | 10553 | 1.09E−03 | 1.15 | 0.357 | 1687 | 5.76E−03 | 1.11 | 0.35 | 2016 |
| C15 | 90440532 | rs6496899 | C | 0.763 | 10468 | 1.09E−03 | 1.17 | 0.79 | 1684 | 1.08E−03 | 1.16 | 0.788 | 2015 |
| C17 | 70257535 | rs9911654 | T | 0.894 | 10635 | 1.09E−03 | 1.25 | 0.913 | 1700 | 2.81E−02 | 1.14 | 0.906 | 2034 |
| C06 | 104259812 | rs9373729 | G | 0.355 | 10623 | 1.09E−03 | 1.14 | 0.386 | 1698 | 2.92E−03 | 1.12 | 0.381 | 2032 |
| C05 | 102509625 | rs34813 | G | 0.709 | 10635 | 1.11E−03 | 1.16 | 0.738 | 1700 | 8.32E−03 | 1.11 | 0.731 | 2034 |
| C11 | 134184293 | rs1289450 | A | 0.196 | 10629 | 1.11E−03 | 1.17 | 0.222 | 1699 | 5.54E−04 | 1.17 | 0.221 | 2033 |
| C12 | 69318052 | rs2584019 | T | 0.89 | 10635 | 1.11E−03 | 1.24 | 0.909 | 1700 | 2.22E−04 | 1.26 | 0.91 | 2034 |
| C12 | 69318052 | rs2584019 | T | 0.89 | 10635 | 1.11E−03 | 1.24 | 0.909 | 1700 | 2.22E−04 | 1.26 | 0.91 | 2034 |
| C08 | 79248604 | rs2369121 | T | 0.72 | 10633 | 1.11E−03 | 1.16 | 0.749 | 1700 | 8.70E−04 | 1.15 | 0.747 | 2034 |
| C07 | 23296885 | rs1542608 | G | 0.192 | 10634 | 1.11E−03 | 1.17 | 0.218 | 1700 | 1.53E−03 | 1.15 | 0.215 | 2034 |
| C10 | 15456765 | rs1925819 | T | 0.558 | 10635 | 1.11E−03 | 1.14 | 0.59 | 1700 | 1.40E−02 | 1.09 | 0.58 | 2034 |
| C18 | 3294629 | rs1791098 | C | 0.761 | 10634 | 1.11E−03 | 1.17 | 0.788 | 1700 | 1.78E−03 | 1.15 | 0.785 | 2034 |
| C07 | 150001841 | rs11762978 | A | 0.654 | 10634 | 1.11E−03 | 1.15 | 0.684 | 1700 | 8.03E−03 | 1.11 | 0.677 | 2034 |
| C02 | 209032070 | rs6435401 | A | 0.334 | 10625 | 1.13E−03 | 1.14 | 0.365 | 1699 | 3.65E−03 | 1.12 | 0.359 | 2033 |
| C02 | 85444821 | rs11675205 | A | 0.254 | 10635 | 1.13E−03 | 1.16 | 0.283 | 1700 | 1.59E−03 | 1.14 | 0.28 | 2034 |
| C05 | 158546004 | rs270654 | G | 0.133 | 10635 | 1.13E−03 | 1.2 | 0.156 | 1700 | 3.21E−03 | 1.17 | 0.152 | 2034 |
| C05 | 158546004 | rs270654 | G | 0.133 | 10635 | 1.13E−03 | 1.2 | 0.156 | 1700 | 3.21E−03 | 1.17 | 0.152 | 2034 |
| CX | 40429441 | rs3013140 | G | 0.931 | 8271 | 1.14E−03 | 1.4 | 0.95 | 1103 | 8.63E−04 | 1.38 | 0.949 | 1316 |
| C10 | 78560604 | rs697171 | G | 0.91 | 10620 | 1.14E−03 | 1.27 | 0.928 | 1697 | 1.04E−02 | 1.19 | 0.923 | 2030 |
| C04 | 134880774 | rs4864193 | C | 0.959 | 10626 | 1.14E−03 | 1.43 | 0.971 | 1700 | 1.87E−04 | 1.42 | 0.971 | 2034 |
| C12 | 119366443 | rs3742049 | C | 0.918 | 10622 | 1.14E−03 | 1.28 | 0.935 | 1699 | 3.44E−04 | 1.29 | 0.936 | 2033 |
| C22 | 32544493 | rs2267296 | G | 0.444 | 10634 | 1.15E−03 | 1.14 | 0.476 | 1700 | 2.30E−03 | 1.12 | 0.472 | 2034 |
| C12 | 9208670 | rs3782677 | C | 0.548 | 10635 | 1.15E−03 | 1.14 | 0.579 | 1700 | 8.35E−03 | 1.1 | 0.572 | 2034 |
| C18 | 39882041 | rs4340393 | T | 0.261 | 10635 | 1.15E−03 | 1.15 | 0.289 | 1700 | 2.80E−03 | 1.13 | 0.285 | 2034 |
| C06 | 162239920 | rs875785 | C | 0.916 | 10613 | 1.15E−03 | 1.28 | 0.933 | 1697 | 3.18E−04 | 1.29 | 0.934 | 2031 |
| C05 | 102428250 | rs3776859 | T | 0.709 | 10634 | 1.15E−03 | 1.15 | 0.738 | 1700 | 8.34E−03 | 1.11 | 0.731 | 2034 |
| C17 | 72288299 | rs191227 | A | 0.572 | 10632 | 1.15E−03 | 1.14 | 0.604 | 1700 | 6.91E−04 | 1.13 | 0.603 | 2033 |
| C07 | 38886642 | rs11763728 | T | 0.332 | 10635 | 1.15E−03 | 1.14 | 0.363 | 1700 | 1.57E−03 | 1.13 | 0.36 | 2034 |
| C05 | 157527972 | rs716376 | G | 0.182 | 10635 | 1.16E−03 | 1.17 | 0.208 | 1700 | 4.51E−03 | 1.14 | 0.203 | 2034 |
| C06 | 156331097 | rs4512236 | T | 0.418 | 10634 | 1.17E−03 | 1.14 | 0.45 | 1700 | 9.53E−04 | 1.13 | 0.448 | 2034 |
| C12 | 111845540 | rs1293758 | A | 0.407 | 10633 | 1.17E−03 | 1.14 | 0.439 | 1700 | 6.83E−04 | 1.13 | 0.438 | 2034 |
| C08 | 32600898 | rs10503927 | T | 0.471 | 10635 | 1.17E−03 | 1.14 | 0.503 | 1700 | 2.49E−03 | 1.12 | 0.498 | 2034 |
| C03 | 175270719 | rs2111956 | G | 0.758 | 10633 | 1.17E−03 | 1.17 | 0.785 | 1700 | 3.73E−02 | 1.09 | 0.774 | 2033 |
| C02 | 229956337 | rs9288639 | C | 0.076 | 10621 | 1.18E−03 | 1.26 | 0.093 | 1697 | 1.45E−02 | 1.18 | 0.088 | 2030 |
| C01 | 148893831 | rs2495392 | G | 0.274 | 10629 | 1.18E−03 | 1.15 | 0.303 | 1700 | 7.91E−03 | 1.11 | 0.296 | 2034 |
| C12 | 9208040 | rs3213831 | T | 0.547 | 10635 | 1.18E−03 | 1.14 | 0.579 | 1700 | 8.97E−03 | 1.1 | 0.571 | 2034 |
| C14 | 63868848 | rs1953416 | T | 0.482 | 10627 | 1.18E−03 | 1.14 | 0.514 | 1698 | 4.79E−03 | 1.11 | 0.508 | 2032 |
| C17 | 37372424 | rs7210156 | G | 0.93 | 10631 | 1.18E−03 | 1.31 | 0.946 | 1699 | 3.16E−03 | 1.25 | 0.943 | 2033 |
| C16 | 63942198 | rs7192142 | T | 0.811 | 10602 | 1.18E−03 | 1.18 | 0.835 | 1693 | 2.07E−03 | 1.16 | 0.833 | 2025 |
| C10 | 15465576 | rs1013406 | C | 0.561 | 10623 | 1.18E−03 | 1.14 | 0.593 | 1697 | 1.35E−02 | 1.1 | 0.583 | 2030 |
| C12 | 65812323 | rs11835794 | T | 0.852 | 10635 | 1.18E−03 | 1.21 | 0.874 | 1700 | 1.80E−04 | 1.22 | 0.876 | 2034 |
| C05 | 57500192 | rs2112905 | A | 0.402 | 10635 | 1.19E−03 | 1.14 | 0.433 | 1700 | 4.16E−03 | 1.11 | 0.427 | 2034 |
| C04 | 33363983 | rs10517277 | C | 0.102 | 10629 | 1.19E−03 | 1.22 | 0.122 | 1700 | 3.92E−03 | 1.18 | 0.118 | 2034 |
| C07 | 37924826 | rs1357648 | T | 0.318 | 10602 | 1.20E−03 | 1.14 | 0.348 | 1691 | 2.54E−03 | 1.12 | 0.344 | 2024 |
| C05 | 158795428 | rs10045431 | A | 0.262 | 10582 | 1.20E−03 | 1.15 | 0.291 | 1675 | 6.80E−03 | 1.12 | 0.285 | 2007 |
| C22 | 32522356 | rs8135273 | C | 0.156 | 10593 | 1.20E−03 | 1.19 | 0.18 | 1689 | 9.52E−03 | 1.14 | 0.174 | 2020 |
| C04 | 162467610 | rs12503470 | T | 0.378 | 10632 | 1.20E−03 | 1.14 | 0.409 | 1700 | 4.24E−03 | 1.11 | 0.403 | 2034 |
| C04 | 162156054 | rs17359034 | T | 0.386 | 10551 | 1.20E−03 | 1.14 | 0.417 | 1700 | 1.11E−02 | 1.1 | 0.409 | 2034 |
| C14 | 51013885 | rs1458114 | T | 0.708 | 10457 | 1.21E−03 | 1.16 | 0.737 | 1653 | 4.43E−03 | 1.12 | 0.732 | 1982 |
| C02 | 127990589 | rs3943703 | C | 0.211 | 10632 | 1.21E−03 | 1.16 | 0.237 | 1700 | 5.13E−04 | 1.16 | 0.237 | 2034 |
| C07 | 85202542 | rs10276025 | T | 0.372 | 10635 | 1.22E−03 | 1.14 | 0.403 | 1700 | 1.23E−03 | 1.13 | 0.401 | 2034 |
| C18 | 58474237 | rs7241455 | T | 0.061 | 10635 | 1.22E−03 | 1.28 | 0.077 | 1700 | 3.10E−04 | 1.29 | 0.078 | 2034 |
| C15 | 90437337 | rs3743369 | T | 0.61 | 10633 | 1.22E−03 | 1.14 | 0.641 | 1700 | 8.00E−04 | 1.13 | 0.639 | 2034 |
| C03 | 72701976 | rs2046867 | T | 0.276 | 10630 | 1.23E−03 | 1.15 | 0.305 | 1700 | 7.59E−03 | 1.11 | 0.298 | 2034 |
| C05 | 52576529 | rs38055 | T | 0.383 | 10632 | 1.23E−03 | 1.14 | 0.414 | 1700 | 3.56E−05 | 1.17 | 0.42 | 2034 |
| C08 | 70844508 | rs7015359 | G | 0.186 | 10635 | 1.23E−03 | 1.17 | 0.211 | 1700 | 1.45E−03 | 1.16 | 0.209 | 2034 |
| C17 | 17015262 | rs4401079 | T | 0.21 | 10418 | 1.23E−03 | 1.17 | 0.236 | 1666 | 3.95E−04 | 1.17 | 0.237 | 1992 |
| C10 | 23293068 | rs10828393 | C | 0.891 | 10615 | 1.23E−03 | 1.24 | 0.91 | 1698 | 3.82E−03 | 1.19 | 0.907 | 2032 |
| C14 | 57437666 | rs11158238 | T | 0.141 | 10634 | 1.24E−03 | 1.19 | 0.163 | 1700 | 3.43E−03 | 1.16 | 0.16 | 2034 |
| C08 | 97736809 | rs1075479 | A | 0.79 | 10601 | 1.24E−03 | 1.17 | 0.815 | 1696 | 7.77E−03 | 1.13 | 0.81 | 2029 |
| C07 | 21550808 | rs4722054 | G | 0.087 | 10596 | 1.25E−03 | 1.24 | 0.105 | 1682 | 1.77E−03 | 1.21 | 0.103 | 2014 |
| C22 | 34890870 | rs763086 | A | 0.299 | 10624 | 1.26E−03 | 1.15 | 0.329 | 1699 | 2.88E−03 | 1.12 | 0.324 | 2031 |
| C19 | 4187996 | rs4740 | A | 0.269 | 10634 | 1.26E−03 | 1.15 | 0.298 | 1700 | 5.17E−03 | 1.12 | 0.292 | 2034 |
| C12 | 75974496 | rs7135414 | A | 0.914 | 10635 | 1.26E−03 | 1.27 | 0.931 | 1700 | 5.96E−03 | 1.21 | 0.928 | 2034 |
| C12 | 75974496 | rs7135414 | A | 0.914 | 10635 | 1.26E−03 | 1.27 | 0.931 | 1700 | 5.96E−03 | 1.21 | 0.928 | 2034 |
| C05 | 5893171 | rs864267 | A | 0.452 | 10633 | 1.26E−03 | 1.14 | 0.483 | 1700 | 1.57E−03 | 1.12 | 0.48 | 2034 |
| C12 | 121504211 | rs10847227 | T | 0.273 | 10616 | 1.26E−03 | 1.15 | 0.301 | 1700 | 1.46E−04 | 1.16 | 0.304 | 2034 |
| C08 | 97440350 | rs1992087 | G | 0.254 | 10635 | 1.27E−03 | 1.15 | 0.282 | 1700 | 2.92E−03 | 1.13 | 0.278 | 2034 |
| C13 | 53306486 | rs1993776 | C | 0.869 | 10635 | 1.28E−03 | 1.22 | 0.889 | 1700 | 2.60E−03 | 1.18 | 0.887 | 2034 |
| C19 | 17080105 | rs7246865 | A | 0.205 | 10635 | 1.28E−03 | 1.17 | 0.231 | 1700 | 3.11E−03 | 1.14 | 0.227 | 2034 |
| C19 | 17080105 | rs7246865 | A | 0.205 | 10635 | 1.28E−03 | 1.17 | 0.231 | 1700 | 3.11E−03 | 1.14 | 0.227 | 2034 |
| C04 | 178163770 | rs309800 | C | 0.798 | 10632 | 1.28E−03 | 1.18 | 0.823 | 1699 | 1.30E−04 | 1.2 | 0.826 | 2033 |
| C05 | 57486156 | rs2962014 | G | 0.501 | 10528 | 1.28E−03 | 1.14 | 0.533 | 1682 | 4.01E−03 | 1.11 | 0.528 | 2015 |
| C19 | 14236895 | rs7248277 | C | 0.563 | 10624 | 1.29E−03 | 1.14 | 0.594 | 1699 | 5.08E−04 | 1.14 | 0.594 | 2033 |
| C01 | 30315299 | rs407838 | C | 0.843 | 10633 | 1.29E−03 | 1.2 | 0.865 | 1700 | 7.85E−04 | 1.19 | 0.865 | 2034 |
| C01 | 184669059 | rs2250227 | C | 0.337 | 10635 | 1.29E−03 | 1.14 | 0.367 | 1700 | 3.60E−03 | 1.12 | 0.362 | 2034 |
| C04 | 17186841 | rs2697684 | G | 0.771 | 10635 | 1.29E−03 | 1.17 | 0.798 | 1700 | 1.46E−03 | 1.15 | 0.795 | 2034 |
| C10 | 85007873 | rs11199316 | C | 0.713 | 10568 | 1.29E−03 | 1.15 | 0.742 | 1687 | 1.02E−04 | 1.17 | 0.745 | 2019 |
| CX | 84984650 | rs2213468 | G | 0.659 | 8219 | 1.30E−03 | 1.18 | 0.696 | 1094 | 2.51E−03 | 1.16 | 0.691 | 1307 |
| CX | 66115625 | rs2031751 | C | 0.941 | 8270 | 1.30E−03 | 1.44 | 0.959 | 1102 | 8.21E−03 | 1.31 | 0.955 | 1315 |
| C02 | 212342079 | rs4442936 | G | 0.282 | 10600 | 1.32E−03 | 1.15 | 0.311 | 1683 | 9.92E−04 | 1.14 | 0.309 | 2016 |
| C01 | 162568432 | rs2651860 | T | 0.786 | 10635 | 1.32E−03 | 1.17 | 0.811 | 1700 | 4.88E−03 | 1.14 | 0.806 | 2033 |
| C02 | 135143228 | rs10496721 | T | 0.17 | 10631 | 1.32E−03 | 1.18 | 0.195 | 1699 | 6.12E−03 | 1.14 | 0.189 | 2033 |
| C04 | 129841129 | rs6534683 | T | 0.646 | 10635 | 1.32E−03 | 1.14 | 0.676 | 1700 | 8.61E−04 | 1.14 | 0.675 | 2034 |
| C05 | 108055795 | rs4957775 | G | 0.832 | 10635 | 1.33E−03 | 1.19 | 0.855 | 1700 | 1.22E−03 | 1.18 | 0.853 | 2034 |
| C13 | 21281716 | rs1887263 | A | 0.204 | 10630 | 1.33E−03 | 1.16 | 0.229 | 1700 | 1.56E−03 | 1.15 | 0.227 | 2034 |
| C12 | 120804218 | rs1169081 | C | 0.692 | 10626 | 1.33E−03 | 1.15 | 0.721 | 1700 | 2.42E−02 | 1.09 | 0.711 | 2034 |
| C11 | 68636018 | rs7111999 | G | 0.47 | 10494 | 1.33E−03 | 1.14 | 0.502 | 1666 | 1.94E−02 | 1.09 | 0.492 | 1994 |
| C13 | 36900184 | rs9566343 | T | 0.814 | 10634 | 1.34E−03 | 1.18 | 0.838 | 1700 | 9.66E−04 | 1.17 | 0.837 | 2034 |
| C12 | 22807863 | rs16925384 | A | 0.194 | 10621 | 1.34E−03 | 1.17 | 0.219 | 1699 | 1.79E−03 | 1.15 | 0.216 | 2033 |
| C01 | 149009396 | rs949969 | T | 0.195 | 10635 | 1.34E−03 | 1.17 | 0.221 | 1700 | 2.26E−03 | 1.15 | 0.218 | 2034 |
| C14 | 64466293 | rs894921 | G | 0.377 | 10633 | 1.35E−03 | 1.14 | 0.408 | 1700 | 1.12E−02 | 1.1 | 0.4 | 2034 |
| C10 | 15419285 | rs11596590 | T | 0.559 | 10634 | 1.35E−03 | 1.14 | 0.59 | 1699 | 1.36E−02 | 1.1 | 0.581 | 2033 |
| C11 | 90747051 | rs1436622 | A | 0.686 | 10633 | 1.35E−03 | 1.15 | 0.715 | 1700 | 5.93E−04 | 1.15 | 0.715 | 2034 |
| C08 | 129199390 | rs10505507 | A | 0.924 | 10634 | 1.36E−03 | 1.29 | 0.94 | 1700 | 5.90E−03 | 1.22 | 0.937 | 2034 |
| C18 | 45713028 | rs1790778 | G | 0.512 | 10632 | 1.36E−03 | 1.13 | 0.544 | 1699 | 1.91E−03 | 1.12 | 0.54 | 2033 |
| C07 | 85200311 | rs2373018 | G | 0.372 | 10635 | 1.36E−03 | 1.14 | 0.403 | 1700 | 1.35E−03 | 1.13 | 0.401 | 2034 |
| C14 | 57397846 | rs10483707 | A | 0.139 | 10635 | 1.36E−03 | 1.19 | 0.161 | 1700 | 5.05E−03 | 1.15 | 0.157 | 2034 |
| C20 | 5929566 | rs236113 | T | 0.883 | 10635 | 1.37E−03 | 1.23 | 0.902 | 1700 | 3.33E−03 | 1.19 | 0.899 | 2034 |
| C07 | 11501113 | rs1526543 | T | 0.829 | 10634 | 1.37E−03 | 1.19 | 0.852 | 1700 | 3.94E−03 | 1.15 | 0.849 | 2034 |
| C05 | 5904611 | rs2617549 | A | 0.453 | 10629 | 1.38E−03 | 1.13 | 0.485 | 1700 | 1.57E−03 | 1.12 | 0.482 | 2034 |
| C02 | 36022756 | rs6747236 | T | 0.716 | 10628 | 1.38E−03 | 1.15 | 0.744 | 1700 | 4.89E−04 | 1.15 | 0.744 | 2034 |
| C10 | 8304796 | rs7092384 | C | 0.86 | 10629 | 1.39E−03 | 1.21 | 0.881 | 1700 | 1.31E−03 | 1.19 | 0.88 | 2034 |
| C02 | 42505686 | rs6724757 | A | 0.09 | 10631 | 1.40E−03 | 1.23 | 0.109 | 1699 | 3.19E−04 | 1.24 | 0.109 | 2033 |
| C05 | 66155122 | rs2441109 | G | 0.162 | 10633 | 1.40E−03 | 1.18 | 0.186 | 1700 | 1.58E−02 | 1.12 | 0.179 | 2034 |
| C10 | 131748550 | rs1484652 | T | 0.905 | 10635 | 1.40E−03 | 1.26 | 0.923 | 1700 | 8.26E−03 | 1.19 | 0.919 | 2034 |
| C16 | 2010569 | rs2286472 | C | 0.269 | 10627 | 1.40E−03 | 1.15 | 0.297 | 1690 | 2.09E−03 | 1.13 | 0.294 | 2024 |
| C05 | 39122462 | rs6880882 | A | 0.352 | 10599 | 1.41E−03 | 1.14 | 0.382 | 1697 | 3.56E−03 | 1.12 | 0.377 | 2028 |
| C16 | 88204775 | rs4075964 | A | 0.284 | 10632 | 1.41E−03 | 1.15 | 0.312 | 1699 | 1.71E−03 | 1.13 | 0.31 | 2033 |
| C09 | 28113402 | rs1452335 | G | 0.715 | 10614 | 1.41E−03 | 1.15 | 0.744 | 1694 | 6.47E−04 | 1.15 | 0.743 | 2027 |
| C14 | 31305463 | rs1950216 | T | 0.798 | 10634 | 1.41E−03 | 1.18 | 0.823 | 1700 | 3.06E−03 | 1.15 | 0.82 | 2034 |
| C13 | 45766462 | rs9526329 | G | 0.921 | 10632 | 1.41E−03 | 1.28 | 0.938 | 1700 | 3.25E−03 | 1.23 | 0.935 | 2034 |
| C11 | 14150002 | rs10832223 | T | 0.837 | 10626 | 1.41E−03 | 1.19 | 0.86 | 1700 | 2.90E−03 | 1.16 | 0.857 | 2034 |
| C02 | 77672532 | rs2861078 | T | 0.57 | 10635 | 1.42E−03 | 1.14 | 0.601 | 1700 | 1.64E−03 | 1.12 | 0.599 | 2034 |
| C01 | 62762582 | rs11208061 | A | 0.345 | 10547 | 1.42E−03 | 1.14 | 0.375 | 1697 | 3.56E−03 | 1.12 | 0.37 | 2029 |
| C20 | 49529811 | rs4811008 | G | 0.461 | 10463 | 1.42E−03 | 1.14 | 0.493 | 1668 | 3.89E−03 | 1.11 | 0.488 | 1996 |
| C14 | 102711567 | rs8016595 | C | 0.754 | 10630 | 1.43E−03 | 1.16 | 0.781 | 1700 | 4.46E−03 | 1.13 | 0.776 | 2034 |
| C01 | 243972662 | rs12068067 | G | 0.776 | 10589 | 1.43E−03 | 1.17 | 0.801 | 1695 | 9.78E−04 | 1.16 | 0.8 | 2029 |
| C10 | 84836912 | rs10787854 | A | 0.83 | 10634 | 1.43E−03 | 1.19 | 0.853 | 1700 | 2.82E−04 | 1.2 | 0.854 | 2034 |
| CX | 55725022 | rs5914778 | G | 0.802 | 8269 | 1.44E−03 | 1.22 | 0.832 | 1103 | 6.95E−04 | 1.22 | 0.831 | 1316 |
| C05 | 102584028 | rs183752 | T | 0.716 | 10621 | 1.44E−03 | 1.15 | 0.744 | 1699 | 1.26E−02 | 1.11 | 0.736 | 2033 |
| C14 | 31304803 | rs991387 | G | 0.799 | 10634 | 1.44E−03 | 1.18 | 0.823 | 1700 | 3.12E−03 | 1.15 | 0.82 | 2034 |
| C11 | 68632276 | rs1005858 | G | 0.47 | 10634 | 1.45E−03 | 1.13 | 0.502 | 1700 | 2.30E−02 | 1.09 | 0.491 | 2034 |
| C03 | 195828700 | rs4497978 | A | 0.667 | 10576 | 1.45E−03 | 1.15 | 0.696 | 1682 | 2.58E−03 | 1.13 | 0.693 | 2012 |
| C05 | 40656007 | rs13175020 | T | 0.816 | 10632 | 1.46E−03 | 1.18 | 0.84 | 1699 | 4.64E−04 | 1.19 | 0.841 | 2033 |
| C01 | 62822706 | rs6696619 | C | 0.662 | 10624 | 1.46E−03 | 1.14 | 0.692 | 1700 | 6.63E−04 | 1.14 | 0.691 | 2034 |
| C13 | 104637486 | rs354438 | T | 0.351 | 10634 | 1.46E−03 | 1.14 | 0.381 | 1700 | 5.60E−04 | 1.14 | 0.381 | 2034 |
| C14 | 64464393 | rs1147437 | G | 0.38 | 10634 | 1.46E−03 | 1.14 | 0.411 | 1700 | 8.57E−03 | 1.1 | 0.403 | 2034 |
| C17 | 295873 | rs4130140 | A | 0.175 | 10635 | 1.46E−03 | 1.17 | 0.199 | 1700 | 4.28E−03 | 1.14 | 0.195 | 2034 |
| C07 | 20937657 | rs2110007 | C | 0.943 | 10606 | 1.47E−03 | 1.34 | 0.956 | 1699 | 1.90E−03 | 1.3 | 0.955 | 2030 |
| C21 | 26986173 | rs2251540 | G | 0.642 | 10633 | 1.47E−03 | 1.14 | 0.672 | 1700 | 7.17E−04 | 1.14 | 0.671 | 2034 |
| C10 | 23311212 | rs993352 | G | 0.883 | 10593 | 1.47E−03 | 1.23 | 0.903 | 1689 | 4.02E−03 | 1.19 | 0.9 | 2021 |
| C04 | 139831081 | rs7669668 | T | 0.119 | 10635 | 1.48E−03 | 1.2 | 0.139 | 1700 | 1.69E−02 | 1.14 | 0.133 | 2034 |
| C02 | 172634850 | rs1399959 | G | 0.669 | 10611 | 1.48E−03 | 1.14 | 0.698 | 1699 | 1.32E−02 | 1.1 | 0.69 | 2032 |
| C06 | 127842427 | rs11154416 | G | 0.76 | 10566 | 1.49E−03 | 1.16 | 0.787 | 1684 | 5.06E−03 | 1.13 | 0.782 | 2016 |
| C12 | 22797592 | rs2171392 | A | 0.194 | 10635 | 1.49E−03 | 1.17 | 0.219 | 1700 | 2.03E−03 | 1.15 | 0.217 | 2034 |
| C07 | 17747986 | rs11766489 | T | 0.423 | 10635 | 1.49E−03 | 1.13 | 0.454 | 1700 | 1.74E−03 | 1.12 | 0.451 | 2034 |
| C20 | 4116799 | rs6084644 | G | 0.436 | 10635 | 1.49E−03 | 1.13 | 0.467 | 1700 | 2.33E−03 | 1.12 | 0.464 | 2034 |
| C20 | 4116799 | rs6084644 | G | 0.436 | 10635 | 1.49E−03 | 1.13 | 0.467 | 1700 | 2.33E−03 | 1.12 | 0.464 | 2034 |
| C18 | 70239058 | rs4892210 | A | 0.192 | 10634 | 1.49E−03 | 1.17 | 0.217 | 1700 | 8.76E−04 | 1.16 | 0.216 | 2034 |
| C01 | 153057498 | rs822519 | A | 0.932 | 10635 | 1.50E−03 | 1.3 | 0.947 | 1700 | 8.24E−04 | 1.3 | 0.946 | 2034 |
| C10 | 5181871 | rs4880711 | C | 0.35 | 10503 | 1.51E−03 | 1.14 | 0.38 | 1693 | 2.56E−02 | 1.09 | 0.37 | 2017 |
| C14 | 101394200 | rs10129841 | A | 0.577 | 10634 | 1.51E−03 | 1.14 | 0.608 | 1700 | 4.86E−03 | 1.11 | 0.603 | 2034 |
| C07 | 38982261 | rs4478468 | G | 0.31 | 10635 | 1.52E−03 | 1.14 | 0.339 | 1700 | 1.46E−03 | 1.13 | 0.337 | 2034 |
| C08 | 39425796 | rs7830101 | T | 0.274 | 10556 | 1.53E−03 | 1.15 | 0.302 | 1686 | 2.51E−03 | 1.13 | 0.299 | 2019 |
| C16 | 19860174 | rs1634675 | G | 0.787 | 10633 | 1.53E−03 | 1.17 | 0.812 | 1700 | 6.40E−03 | 1.13 | 0.807 | 2034 |
| C08 | 75342454 | rs6996971 | C | 0.476 | 10603 | 1.53E−03 | 1.13 | 0.507 | 1698 | 2.99E−04 | 1.14 | 0.509 | 2032 |
| C05 | 102596616 | rs26821 | A | 0.709 | 10628 | 1.53E−03 | 1.15 | 0.737 | 1697 | 1.27E−02 | 1.11 | 0.73 | 2031 |
| C19 | 330003 | rs10414677 | T | 0.276 | 10341 | 1.53E−03 | 1.15 | 0.305 | 1639 | 1.23E−02 | 1.11 | 0.297 | 1956 |
| C14 | 64458802 | rs1147443 | T | 0.254 | 10635 | 1.54E−03 | 1.15 | 0.281 | 1700 | 2.45E−03 | 1.13 | 0.278 | 2034 |
| C11 | 108465132 | rs1509729 | G | 0.688 | 10634 | 1.55E−03 | 1.15 | 0.717 | 1700 | 9.48E−03 | 1.11 | 0.71 | 2034 |
| C05 | 176640493 | rs7724098 | G | 0.289 | 10627 | 1.55E−03 | 1.14 | 0.317 | 1697 | 2.16E−03 | 1.13 | 0.314 | 2031 |
| C03 | 54346463 | rs1530733 | A | 0.156 | 10635 | 1.55E−03 | 1.18 | 0.179 | 1700 | 3.48E−03 | 1.15 | 0.176 | 2034 |
| C16 | 88263882 | rs904800 | G | 0.38 | 10635 | 1.55E−03 | 1.14 | 0.411 | 1700 | 5.04E−03 | 1.11 | 0.405 | 2034 |
| C22 | 34142107 | rs2071744 | C | 0.78 | 10607 | 1.55E−03 | 1.17 | 0.806 | 1700 | 5.19E−03 | 1.13 | 0.801 | 2033 |
| C07 | 136071365 | rs324594 | C | 0.27 | 10632 | 1.55E−03 | 1.15 | 0.298 | 1700 | 7.92E−04 | 1.14 | 0.297 | 2034 |
| C07 | 102722901 | rs10282605 | C | 0.187 | 10633 | 1.55E−03 | 1.17 | 0.211 | 1700 | 9.25E−03 | 1.13 | 0.206 | 2034 |
| C04 | 37546025 | rs3849018 | T | 0.279 | 10635 | 1.55E−03 | 1.15 | 0.307 | 1700 | 2.28E−03 | 1.13 | 0.304 | 2033 |
| C05 | 97972689 | rs168654 | T | 0.636 | 10630 | 1.56E−03 | 1.14 | 0.665 | 1699 | 5.48E−03 | 1.11 | 0.66 | 2033 |
| C06 | 28156603 | rs203877 | T | 0.729 | 10461 | 1.56E−03 | 1.16 | 0.757 | 1666 | 1.78E−02 | 1.1 | 0.748 | 1992 |
| C05 | 76862558 | rs251470 | T | 0.439 | 10634 | 1.56E−03 | 1.13 | 0.47 | 1700 | 6.26E−03 | 1.11 | 0.464 | 2034 |
| C09 | 122155539 | rs3780680 | T | 0.452 | 10610 | 1.56E−03 | 1.13 | 0.483 | 1698 | 2.92E−02 | 1.08 | 0.472 | 2031 |
| C04 | 10657250 | rs10011464 | T | 0.462 | 10581 | 1.56E−03 | 1.13 | 0.493 | 1685 | 2.56E−03 | 1.12 | 0.49 | 2016 |
| C08 | 143147459 | rs11167132 | T | 0.218 | 10568 | 1.57E−03 | 1.16 | 0.244 | 1693 | 3.04E−03 | 1.14 | 0.241 | 2027 |
| CX | 66121372 | rs1576059 | T | 0.941 | 8273 | 1.57E−03 | 1.43 | 0.958 | 1103 | 9.27E−03 | 1.31 | 0.954 | 1316 |
| C16 | 82550760 | rs10514560 | C | 0.732 | 10632 | 1.57E−03 | 1.15 | 0.759 | 1700 | 2.17E−03 | 1.14 | 0.756 | 2034 |
| C20 | 57601797 | rs486921 | C | 0.853 | 10634 | 1.58E−03 | 1.2 | 0.875 | 1699 | 7.96E−04 | 1.2 | 0.875 | 2033 |
| C17 | 14301595 | rs2856177 | A | 0.622 | 10630 | 1.59E−03 | 1.14 | 0.652 | 1700 | 6.13E−03 | 1.11 | 0.647 | 2034 |
| C01 | 63947687 | rs2806535 | A | 0.487 | 10630 | 1.59E−03 | 1.13 | 0.519 | 1700 | 2.11E−03 | 1.12 | 0.515 | 2034 |
| C22 | 18426996 | rs3804044 | T | 0.858 | 10612 | 1.59E−03 | 1.2 | 0.879 | 1700 | 4.62E−03 | 1.17 | 0.876 | 2034 |
| C19 | 50054507 | rs377702 | T | 0.384 | 10629 | 1.61E−03 | 1.14 | 0.415 | 1700 | 1.75E−03 | 1.12 | 0.412 | 2034 |
| C18 | 55144365 | rs4806 | C | 0.284 | 10592 | 1.61E−03 | 1.15 | 0.312 | 1686 | 4.58E−03 | 1.12 | 0.307 | 2020 |
| C08 | 75320456 | rs4410896 | A | 0.479 | 10635 | 1.61E−03 | 1.13 | 0.51 | 1700 | 4.67E−04 | 1.14 | 0.511 | 2034 |
| C13 | 43040379 | rs963569 | A | 0.748 | 10635 | 1.61E−03 | 1.16 | 0.775 | 1700 | 4.72E−02 | 1.09 | 0.764 | 2034 |
| CX | 69317680 | rs2341629 | C | 0.467 | 8261 | 1.62E−03 | 1.16 | 0.505 | 1103 | 1.66E−03 | 1.15 | 0.502 | 1316 |
| C11 | 17901963 | rs2237921 | C | 0.156 | 10634 | 1.62E−03 | 1.18 | 0.179 | 1700 | 6.00E−03 | 1.14 | 0.174 | 2034 |
| C10 | 84972068 | rs11199158 | G | 0.608 | 10635 | 1.62E−03 | 1.14 | 0.638 | 1700 | 2.26E−05 | 1.17 | 0.645 | 2034 |
| C11 | 1390257 | rs4963076 | T | 0.482 | 10635 | 1.62E−03 | 1.13 | 0.513 | 1700 | 3.68E−03 | 1.11 | 0.508 | 2034 |
| C11 | 1390257 | rs4963076 | T | 0.482 | 10635 | 1.62E−03 | 1.13 | 0.513 | 1700 | 3.68E−03 | 1.11 | 0.508 | 2034 |
| C15 | 52585989 | rs2553223 | G | 0.346 | 10626 | 1.62E−03 | 1.14 | 0.376 | 1700 | 6.64E−04 | 1.14 | 0.376 | 2034 |
| C08 | 32791061 | rs16880128 | G | 0.446 | 10541 | 1.63E−03 | 1.13 | 0.477 | 1693 | 9.35E−03 | 1.1 | 0.47 | 2026 |
| C08 | 96746644 | rs3134517 | G | 0.64 | 10632 | 1.63E−03 | 1.14 | 0.67 | 1700 | 7.83E−03 | 1.11 | 0.663 | 2033 |
| C01 | 55121739 | rs4927219 | G | 0.601 | 10635 | 1.63E−03 | 1.14 | 0.631 | 1700 | 3.78E−04 | 1.14 | 0.632 | 2034 |
| C14 | 19276803 | rs10872856 | C | 0.912 | 10635 | 1.63E−03 | 1.26 | 0.929 | 1700 | 1.56E−02 | 1.18 | 0.924 | 2034 |
| C01 | 148792338 | rs1970568 | T | 0.813 | 10634 | 1.64E−03 | 1.18 | 0.837 | 1700 | 2.08E−03 | 1.16 | 0.835 | 2034 |
| C12 | 20945636 | rs3764006 | C | 0.104 | 10626 | 1.64E−03 | 1.21 | 0.123 | 1700 | 8.33E−04 | 1.21 | 0.123 | 2034 |
| C18 | 45732885 | rs1557359 | G | 0.407 | 10635 | 1.64E−03 | 1.13 | 0.437 | 1700 | 1.64E−03 | 1.12 | 0.435 | 2034 |
| C17 | 14260485 | rs2323095 | A | 0.663 | 10635 | 1.64E−03 | 1.14 | 0.692 | 1700 | 3.76E−03 | 1.12 | 0.688 | 2034 |
| C08 | 96745945 | rs3102526 | G | 0.64 | 10635 | 1.65E−03 | 1.14 | 0.669 | 1700 | 7.97E−03 | 1.11 | 0.663 | 2034 |
| C04 | 96606254 | rs6850673 | C | 0.141 | 10631 | 1.65E−03 | 1.19 | 0.163 | 1700 | 2.15E−03 | 1.17 | 0.161 | 2034 |
| C22 | 18449129 | rs2073778 | C | 0.858 | 10635 | 1.65E−03 | 1.2 | 0.879 | 1700 | 5.41E−03 | 1.16 | 0.875 | 2034 |
| C07 | 116742325 | rs2027945 | A | 0.19 | 10631 | 1.66E−03 | 1.17 | 0.215 | 1700 | 1.06E−03 | 1.16 | 0.214 | 2034 |
| C07 | 144298585 | rs7791708 | A | 0.677 | 10614 | 1.66E−03 | 1.14 | 0.706 | 1693 | 4.26E−03 | 1.12 | 0.701 | 2027 |
| C05 | 3977821 | rs11956711 | C | 0.066 | 10608 | 1.66E−03 | 1.26 | 0.082 | 1698 | 4.36E−03 | 1.22 | 0.08 | 2031 |
| C05 | 39071087 | rs9292729 | C | 0.338 | 10625 | 1.66E−03 | 1.14 | 0.368 | 1700 | 6.86E−03 | 1.11 | 0.362 | 2034 |
| C01 | 29291511 | rs10799128 | T | 0.196 | 10601 | 1.66E−03 | 1.16 | 0.221 | 1686 | 3.50E−03 | 1.14 | 0.217 | 2020 |
| C05 | 3595286 | rs1697997 | A | 0.914 | 10635 | 1.66E−03 | 1.26 | 0.931 | 1700 | 5.14E−03 | 1.21 | 0.928 | 2034 |
| C02 | 215347262 | rs10932529 | A | 0.247 | 10635 | 1.67E−03 | 1.15 | 0.274 | 1700 | 1.88E−03 | 1.14 | 0.272 | 2034 |
| CX | 6130802 | rs7892324 | C | 0.165 | 8255 | 1.67E−03 | 1.22 | 0.194 | 1093 | 3.38E−03 | 1.19 | 0.19 | 1306 |
| C12 | 62930978 | rs10878126 | G | 0.493 | 10634 | 1.67E−03 | 1.13 | 0.524 | 1700 | 6.47E−04 | 1.13 | 0.525 | 2034 |
| C04 | 44935711 | rs6447404 | G | 0.617 | 10633 | 1.67E−03 | 1.14 | 0.647 | 1700 | 1.07E−02 | 1.1 | 0.639 | 2034 |
| C03 | 145977000 | rs10935523 | A | 0.741 | 10632 | 1.68E−03 | 1.16 | 0.767 | 1700 | 7.37E−03 | 1.12 | 0.762 | 2034 |
| C16 | 79586916 | rs7188879 | T | 0.964 | 10629 | 1.69E−03 | 1.45 | 0.975 | 1699 | 7.87E−03 | 1.33 | 0.972 | 2033 |
| C05 | 159076573 | rs889053 | T | 0.162 | 10634 | 1.69E−03 | 1.18 | 0.185 | 1700 | 3.69E−03 | 1.15 | 0.182 | 2034 |
| C11 | 91175735 | rs808003 | T | 0.554 | 10634 | 1.69E−03 | 1.13 | 0.585 | 1700 | 2.62E−02 | 1.09 | 0.574 | 2034 |
| C17 | 14258654 | rs12449610 | T | 0.669 | 10635 | 1.69E−03 | 1.14 | 0.697 | 1700 | 3.74E−03 | 1.12 | 0.693 | 2034 |
| C04 | 3811449 | rs6822427 | A | 0.487 | 10635 | 1.70E−03 | 1.13 | 0.518 | 1700 | 2.14E−03 | 1.12 | 0.515 | 2034 |
| C01 | 218934430 | rs1519463 | G | 0.5 | 10635 | 1.70E−03 | 1.13 | 0.531 | 1700 | 4.65E−04 | 1.14 | 0.532 | 2034 |
| C02 | 183549717 | rs4666836 | T | 0.22 | 10633 | 1.70E−03 | 1.16 | 0.246 | 1699 | 3.72E−03 | 1.13 | 0.242 | 2033 |
| C03 | 62438401 | rs1452075 | T | 0.734 | 10549 | 1.70E−03 | 1.15 | 0.761 | 1692 | 5.65E−03 | 1.12 | 0.756 | 2019 |
| C03 | 62438401 | rs1452075 | T | 0.734 | 10549 | 1.70E−03 | 1.15 | 0.761 | 1692 | 5.65E−03 | 1.12 | 0.756 | 2019 |
| C10 | 15387774 | rs2138930 | T | 0.11 | 10620 | 1.71E−03 | 1.21 | 0.129 | 1700 | 8.36E−03 | 1.16 | 0.125 | 2033 |
| C12 | 6177827 | rs797773 | T | 0.394 | 10607 | 1.71E−03 | 1.13 | 0.425 | 1698 | 2.20E−03 | 1.12 | 0.422 | 2031 |
| C12 | 52152886 | rs784563 | G | 0.441 | 10631 | 1.71E−03 | 1.13 | 0.472 | 1700 | 1.70E−03 | 1.12 | 0.47 | 2034 |
| C01 | 113143551 | rs773588 | C | 0.276 | 10632 | 1.71E−03 | 1.15 | 0.304 | 1700 | 2.18E−04 | 1.16 | 0.307 | 2034 |
| C05 | 66177928 | rs39700 | G | 0.374 | 10631 | 1.72E−03 | 1.13 | 0.404 | 1699 | 1.04E−02 | 1.1 | 0.396 | 2033 |
| C11 | 132266819 | rs1395504 | A | 0.493 | 10634 | 1.72E−03 | 1.13 | 0.524 | 1700 | 3.66E−03 | 1.11 | 0.519 | 2034 |
| C18 | 70236272 | rs1365254 | T | 0.192 | 10634 | 1.73E−03 | 1.16 | 0.217 | 1700 | 1.01E−03 | 1.16 | 0.216 | 2034 |
| C05 | 38927129 | rs525735 | C | 0.774 | 10632 | 1.74E−03 | 1.16 | 0.799 | 1699 | 1.06E−02 | 1.12 | 0.793 | 2033 |
| C08 | 75357793 | rs10283076 | G | 0.477 | 10627 | 1.74E−03 | 1.13 | 0.507 | 1698 | 3.56E−04 | 1.14 | 0.509 | 2032 |
| C10 | 108671040 | rs1337430 | A | 0.788 | 10215 | 1.74E−03 | 1.17 | 0.813 | 1685 | 9.30E−03 | 1.13 | 0.807 | 2014 |
| C18 | 55191365 | rs9319948 | A | 0.283 | 10629 | 1.75E−03 | 1.14 | 0.311 | 1700 | 4.57E−03 | 1.12 | 0.306 | 2034 |
| C15 | 30230982 | rs4779984 | G | 0.1 | 10616 | 1.75E−03 | 1.22 | 0.119 | 1698 | 9.51E−04 | 1.21 | 0.119 | 2032 |
| C10 | 33672138 | rs2804453 | G | 0.111 | 10621 | 1.76E−03 | 1.21 | 0.131 | 1697 | 8.26E−03 | 1.16 | 0.126 | 2030 |
| C21 | 40710416 | rs1882757 | A | 0.506 | 10635 | 1.76E−03 | 1.13 | 0.536 | 1700 | 2.56E−03 | 1.12 | 0.533 | 2034 |
| C06 | 39827103 | rs9471170 | G | 0.885 | 10635 | 1.77E−03 | 1.22 | 0.904 | 1700 | 1.10E−02 | 1.16 | 0.899 | 2034 |
| C09 | 28154645 | rs2044961 | G | 0.571 | 10633 | 1.77E−03 | 1.13 | 0.601 | 1700 | 7.85E−04 | 1.13 | 0.601 | 2034 |
| C10 | 94841520 | rs10509657 | C | 0.099 | 10631 | 1.78E−03 | 1.22 | 0.118 | 1700 | 3.06E−03 | 1.19 | 0.115 | 2034 |
| C12 | 101677726 | rs2247919 | T | 0.738 | 10635 | 1.79E−03 | 1.15 | 0.764 | 1700 | 6.44E−03 | 1.12 | 0.759 | 2034 |
| C02 | 11287711 | rs7608117 | T | 0.486 | 10629 | 1.79E−03 | 1.13 | 0.517 | 1698 | 4.79E−03 | 1.11 | 0.512 | 2031 |
| C01 | 31037636 | rs12126748 | A | 0.345 | 10489 | 1.79E−03 | 1.14 | 0.375 | 1665 | 5.15E−03 | 1.11 | 0.37 | 1995 |
| C03 | 63602103 | rs7653410 | C | 0.319 | 10620 | 1.79E−03 | 1.14 | 0.348 | 1700 | 1.04E−02 | 1.1 | 0.341 | 2034 |
| C17 | 70788248 | rs2041088 | A | 0.719 | 10633 | 1.79E−03 | 1.15 | 0.747 | 1700 | 3.91E−03 | 1.13 | 0.743 | 2034 |
| C06 | 36751662 | rs1864750 | G | 0.248 | 10635 | 1.80E−03 | 1.15 | 0.275 | 1700 | 2.56E−04 | 1.16 | 0.277 | 2034 |
| C11 | 12971706 | rs3935878 | T | 0.738 | 10634 | 1.80E−03 | 1.15 | 0.764 | 1700 | 5.90E−03 | 1.12 | 0.76 | 2034 |
| C12 | 67493429 | rs1470383 | C | 0.143 | 10634 | 1.81E−03 | 1.18 | 0.165 | 1700 | 3.15E−03 | 1.16 | 0.162 | 2034 |
| C05 | 56037706 | rs7720499 | T | 0.112 | 10635 | 1.81E−03 | 1.2 | 0.132 | 1700 | 3.98E−03 | 1.17 | 0.129 | 2033 |
| C02 | 172714469 | rs951917 | T | 0.387 | 10634 | 1.83E−03 | 1.13 | 0.417 | 1700 | 1.00E−02 | 1.1 | 0.41 | 2034 |
| C03 | 6764265 | rs12491369 | G | 0.571 | 10634 | 1.83E−03 | 1.13 | 0.601 | 1700 | 9.89E−04 | 1.13 | 0.601 | 2034 |
| C06 | 79771759 | rs9352688 | A | 0.623 | 10384 | 1.83E−03 | 1.14 | 0.653 | 1662 | 2.17E−03 | 1.12 | 0.65 | 1991 |
| C19 | 446722 | rs758503 | T | 0.294 | 10524 | 1.83E−03 | 1.14 | 0.323 | 1686 | 3.19E−03 | 1.12 | 0.319 | 2018 |
| C03 | 74187210 | rs4234186 | A | 0.943 | 10617 | 1.83E−03 | 1.33 | 0.957 | 1697 | 8.06E−03 | 1.25 | 0.954 | 2031 |
| C07 | 16590961 | rs706076 | G | 0.146 | 10619 | 1.83E−03 | 1.18 | 0.169 | 1700 | 1.53E−02 | 1.13 | 0.162 | 2034 |
| C02 | 76317619 | rs7563058 | C | 0.504 | 10635 | 1.84E−03 | 1.13 | 0.535 | 1700 | 2.64E−03 | 1.12 | 0.531 | 2034 |
| C04 | 175513600 | rs4695894 | G | 0.5 | 10632 | 1.84E−03 | 1.13 | 0.53 | 1700 | 2.89E−03 | 1.11 | 0.527 | 2034 |
| C17 | 18352177 | rs2746025 | G | 0.641 | 10635 | 1.84E−03 | 1.14 | 0.671 | 1700 | 2.07E−03 | 1.13 | 0.668 | 2034 |
| C07 | 28337194 | rs10486588 | G | 0.231 | 10616 | 1.85E−03 | 1.15 | 0.258 | 1694 | 3.86E−03 | 1.13 | 0.254 | 2028 |
| C06 | 39793734 | rs9296304 | A | 0.498 | 10635 | 1.85E−03 | 1.13 | 0.529 | 1700 | 9.98E−03 | 1.1 | 0.521 | 2034 |
| C13 | 20599475 | rs4770252 | A | 0.802 | 10626 | 1.86E−03 | 1.17 | 0.826 | 1699 | 4.75E−04 | 1.18 | 0.827 | 2032 |
| C04 | 178964553 | rs1983130 | G | 0.414 | 10625 | 1.86E−03 | 1.13 | 0.444 | 1699 | 3.12E−03 | 1.12 | 0.441 | 2033 |
| C02 | 39306628 | rs2168043 | C | 0.868 | 10635 | 1.86E−03 | 1.21 | 0.888 | 1700 | 2.99E−03 | 1.18 | 0.886 | 2034 |
| C06 | 105791384 | rs1149305 | T | 0.059 | 10544 | 1.87E−03 | 1.28 | 0.074 | 1695 | 1.49E−03 | 1.26 | 0.074 | 2027 |
| C22 | 18432012 | rs5992507 | A | 0.858 | 10633 | 1.87E−03 | 1.2 | 0.879 | 1700 | 5.40E−03 | 1.16 | 0.876 | 2034 |
| C13 | 53208642 | rs2050972 | C | 0.878 | 10635 | 1.87E−03 | 1.22 | 0.897 | 1700 | 6.10E−03 | 1.17 | 0.894 | 2034 |
| C03 | 66633156 | rs1563545 | G | 0.523 | 10562 | 1.87E−03 | 1.13 | 0.553 | 1681 | 2.31E−03 | 1.12 | 0.55 | 2014 |
| C03 | 7494813 | rs752299 | T | 0.685 | 10633 | 1.87E−03 | 1.14 | 0.714 | 1700 | 3.80E−03 | 1.12 | 0.71 | 2034 |
| C01 | 173267354 | rs12033847 | T | 0.462 | 10416 | 1.88E−03 | 1.13 | 0.493 | 1668 | 2.13E−03 | 1.12 | 0.49 | 1988 |
| C07 | 147146014 | rs10271435 | A | 0.06 | 10631 | 1.88E−03 | 1.27 | 0.076 | 1700 | 2.74E−04 | 1.3 | 0.077 | 2034 |
| C03 | 29295798 | rs13079598 | A | 0.905 | 10635 | 1.88E−03 | 1.25 | 0.923 | 1700 | 5.92E−03 | 1.2 | 0.92 | 2034 |
| CX | 143133206 | rs11094472 | G | 0.787 | 8262 | 1.89E−03 | 1.21 | 0.817 | 1098 | 3.45E−03 | 1.18 | 0.814 | 1311 |
| C07 | 116708662 | rs2237724 | A | 0.19 | 10635 | 1.90E−03 | 1.16 | 0.215 | 1700 | 1.21E−03 | 1.16 | 0.214 | 2034 |
| C07 | 116694598 | rs10487368 | T | 0.19 | 10635 | 1.90E−03 | 1.16 | 0.215 | 1700 | 1.21E−03 | 1.16 | 0.214 | 2034 |
| C07 | 116702050 | rs2237723 | T | 0.193 | 10630 | 1.90E−03 | 1.16 | 0.217 | 1700 | 1.05E−03 | 1.16 | 0.217 | 2034 |
| C02 | 173071729 | rs6718013 | G | 0.538 | 10634 | 1.91E−03 | 1.13 | 0.568 | 1700 | 1.45E−02 | 1.09 | 0.56 | 2034 |
| C03 | 7486375 | rs779710 | A | 0.589 | 10634 | 1.91E−03 | 1.13 | 0.619 | 1700 | 1.47E−03 | 1.13 | 0.617 | 2034 |
| C07 | 38853360 | rs1525798 | G | 0.303 | 10635 | 1.91E−03 | 1.14 | 0.331 | 1700 | 1.23E−03 | 1.13 | 0.33 | 2034 |
| C18 | 53741253 | rs4940582 | G | 0.849 | 10631 | 1.91E−03 | 1.19 | 0.871 | 1699 | 1.77E−03 | 1.18 | 0.869 | 2033 |
| C04 | 161577318 | rs17326199 | G | 0.211 | 10634 | 1.91E−03 | 1.16 | 0.237 | 1699 | 8.27E−03 | 1.12 | 0.231 | 2033 |
| C13 | 20622774 | rs9285207 | C | 0.195 | 10608 | 1.91E−03 | 1.16 | 0.22 | 1693 | 1.89E−03 | 1.15 | 0.218 | 2026 |
| C08 | 75316641 | rs10504576 | A | 0.478 | 10551 | 1.92E−03 | 1.13 | 0.509 | 1677 | 5.94E−04 | 1.13 | 0.51 | 2010 |
| C14 | 92766673 | rs2281519 | T | 0.257 | 10633 | 1.92E−03 | 1.15 | 0.284 | 1700 | 5.98E−03 | 1.12 | 0.279 | 2033 |
| C15 | 37096582 | rs1841441 | A | 0.466 | 10634 | 1.92E−03 | 1.13 | 0.496 | 1700 | 1.15E−03 | 1.13 | 0.496 | 2034 |
| C10 | 84843813 | rs2349157 | G | 0.855 | 10635 | 1.93E−03 | 1.2 | 0.876 | 1700 | 9.76E−04 | 1.19 | 0.876 | 2034 |
| C05 | 167524725 | rs2617972 | C | 0.808 | 10632 | 1.93E−03 | 1.17 | 0.832 | 1699 | 9.94E−03 | 1.13 | 0.827 | 2033 |
| C12 | 96022770 | rs989264 | A | 0.041 | 10635 | 1.93E−03 | 1.33 | 0.054 | 1700 | 1.86E−03 | 1.31 | 0.053 | 2034 |
| C06 | 10958298 | rs9357002 | T | 0.631 | 10453 | 1.93E−03 | 1.14 | 0.66 | 1678 | 4.72E−03 | 1.11 | 0.655 | 2007 |
| C04 | 147453178 | rs723794 | T | 0.764 | 10635 | 1.94E−03 | 1.16 | 0.79 | 1700 | 5.53E−03 | 1.13 | 0.785 | 2034 |
| C11 | 127732102 | rs7933433 | G | 0.648 | 10633 | 1.95E−03 | 1.14 | 0.677 | 1699 | 1.69E−03 | 1.13 | 0.675 | 2033 |
| C03 | 5243162 | rs341973 | A | 0.51 | 10634 | 1.95E−03 | 1.13 | 0.541 | 1700 | 1.92E−02 | 1.09 | 0.531 | 2034 |
| C22 | 18482283 | rs2238798 | G | 0.858 | 10633 | 1.95E−03 | 1.2 | 0.879 | 1700 | 6.39E−03 | 1.16 | 0.875 | 2034 |
| C10 | 65928341 | rs990828 | G | 0.416 | 10631 | 1.96E−03 | 1.13 | 0.446 | 1700 | 1.23E−02 | 1.1 | 0.438 | 2034 |
| C08 | 143121983 | rs7834060 | G | 0.434 | 10635 | 1.96E−03 | 1.13 | 0.464 | 1700 | 2.51E−03 | 1.12 | 0.461 | 2034 |
| C01 | 184791657 | rs1339083 | A | 0.34 | 10635 | 1.96E−03 | 1.14 | 0.369 | 1700 | 1.56E−03 | 1.13 | 0.367 | 2034 |
| C11 | 92444704 | rs9971402 | C | 0.872 | 10633 | 1.96E−03 | 1.21 | 0.892 | 1700 | 5.09E−04 | 1.22 | 0.893 | 2034 |
| C04 | 162770424 | rs958247 | A | 0.287 | 10626 | 1.96E−03 | 1.14 | 0.315 | 1700 | 1.64E−03 | 1.13 | 0.314 | 2034 |
| C07 | 150069821 | rs2373885 | A | 0.75 | 10631 | 1.96E−03 | 1.16 | 0.776 | 1700 | 8.98E−04 | 1.15 | 0.776 | 2034 |
| C08 | 139195557 | rs4909759 | T | 0.261 | 10633 | 1.96E−03 | 1.15 | 0.288 | 1700 | 7.46E−03 | 1.12 | 0.282 | 2034 |
| C02 | 209737500 | rs9288396 | T | 0.204 | 10635 | 1.97E−03 | 1.16 | 0.229 | 1700 | 1.09E−03 | 1.15 | 0.229 | 2034 |
| C01 | 40520068 | rs7524868 | G | 0.306 | 10611 | 1.97E−03 | 1.14 | 0.334 | 1691 | 3.27E−02 | 1.09 | 0.324 | 2024 |
| C06 | 79657373 | rs10943606 | G | 0.746 | 10632 | 1.97E−03 | 1.15 | 0.772 | 1700 | 1.90E−03 | 1.14 | 0.771 | 2033 |
| C16 | 2014037 | rs3785284 | C | 0.265 | 10571 | 1.98E−03 | 1.15 | 0.292 | 1698 | 2.17E−03 | 1.13 | 0.29 | 2032 |
| C02 | 215385419 | rs2216544 | A | 0.253 | 10635 | 1.98E−03 | 1.15 | 0.28 | 1700 | 2.46E−03 | 1.13 | 0.278 | 2034 |
| C17 | 53871788 | rs9895713 | G | 0.176 | 10633 | 1.98E−03 | 1.17 | 0.2 | 1700 | 2.03E−03 | 1.15 | 0.198 | 2034 |
| C03 | 2631107 | rs17565216 | C | 0.363 | 10631 | 1.98E−03 | 1.13 | 0.392 | 1699 | 9.20E−04 | 1.13 | 0.392 | 2033 |
| C07 | 116815796 | rs213987 | T | 0.327 | 10632 | 1.99E−03 | 1.14 | 0.356 | 1700 | 3.83E−03 | 1.12 | 0.352 | 2034 |
| C07 | 116815796 | rs213987 | T | 0.327 | 10632 | 1.99E−03 | 1.14 | 0.356 | 1700 | 3.83E−03 | 1.12 | 0.352 | 2034 |
| C03 | 175238582 | rs1549100 | T | 0.73 | 10574 | 1.99E−03 | 1.15 | 0.757 | 1693 | 3.92E−02 | 1.09 | 0.747 | 2026 |
| C06 | 69212746 | rs9454539 | A | 0.877 | 10630 | 1.99E−03 | 1.21 | 0.896 | 1699 | 9.23E−04 | 1.21 | 0.896 | 2033 |
| C01 | 65243954 | rs3818513 | C | 0.457 | 10635 | 1.99E−03 | 1.13 | 0.488 | 1700 | 5.23E−02 | 1.07 | 0.475 | 2034 |
| C12 | 123074369 | rs4765477 | T | 0.044 | 10632 | 1.99E−03 | 1.32 | 0.057 | 1700 | 3.78E−03 | 1.27 | 0.056 | 2034 |
| C02 | 118543519 | rs1477451 | T | 0.124 | 10635 | 2.00E−03 | 1.19 | 0.145 | 1700 | 2.98E−02 | 1.12 | 0.137 | 2034 |
| C08 | 143128135 | rs3934930 | T | 0.433 | 10635 | 2.00E−03 | 1.13 | 0.464 | 1700 | 2.52E−03 | 1.12 | 0.461 | 2034 |
| C07 | 81287212 | rs6467882 | C | 0.165 | 10631 | 2.00E−03 | 1.17 | 0.188 | 1700 | 1.69E−03 | 1.16 | 0.187 | 2034 |
| C01 | 155206460 | rs4971177 | T | 0.913 | 10634 | 2.00E−03 | 1.26 | 0.93 | 1700 | 8.85E−04 | 1.26 | 0.93 | 2034 |
| C06 | 119414590 | rs1360782 | A | 0.348 | 10634 | 2.01E−03 | 1.13 | 0.378 | 1700 | 1.44E−03 | 1.13 | 0.376 | 2034 |
| C07 | 50023386 | rs12669163 | C | 0.297 | 10525 | 2.01E−03 | 1.14 | 0.325 | 1675 | 5.51E−03 | 1.12 | 0.321 | 2004 |
| C07 | 16810246 | rs1029577 | T | 0.319 | 10625 | 2.02E−03 | 1.14 | 0.347 | 1697 | 2.55E−03 | 1.12 | 0.345 | 2031 |
| C04 | 82277070 | rs10026120 | T | 0.96 | 10616 | 2.03E−03 | 1.4 | 0.971 | 1699 | 7.73E−03 | 1.31 | 0.969 | 2029 |
| C05 | 133913277 | rs1476097 | T | 0.656 | 10629 | 2.03E−03 | 1.14 | 0.685 | 1699 | 4.64E−04 | 1.15 | 0.686 | 2032 |
| C07 | 13679025 | rs3735344 | G | 0.421 | 10583 | 2.04E−03 | 1.13 | 0.451 | 1687 | 7.75E−03 | 1.1 | 0.445 | 2021 |
| C17 | 7878493 | rs4791759 | A | 0.038 | 10621 | 2.05E−03 | 1.34 | 0.051 | 1699 | 5.53E−03 | 1.28 | 0.048 | 2031 |
| C05 | 14319911 | rs30616 | T | 0.852 | 10634 | 2.05E−03 | 1.19 | 0.873 | 1700 | 4.29E−03 | 1.16 | 0.87 | 2034 |
| C06 | 143011798 | rs478884 | A | 0.879 | 10635 | 2.05E−03 | 1.22 | 0.898 | 1700 | 7.23E−04 | 1.22 | 0.898 | 2034 |
| C14 | 47996364 | rs12436454 | T | 0.713 | 10592 | 2.05E−03 | 1.15 | 0.74 | 1697 | 4.47E−03 | 1.12 | 0.736 | 2030 |
| C09 | 23762277 | rs7038525 | T | 0.152 | 10629 | 2.05E−03 | 1.18 | 0.174 | 1700 | 4.27E−04 | 1.19 | 0.176 | 2034 |
| C06 | 69230698 | rs10806602 | T | 0.877 | 10614 | 2.06E−03 | 1.21 | 0.897 | 1696 | 1.03E−03 | 1.21 | 0.897 | 2030 |
| C11 | 17918391 | rs3993323 | A | 0.14 | 10630 | 2.06E−03 | 1.18 | 0.162 | 1697 | 5.18E−03 | 1.15 | 0.159 | 2031 |
| C18 | 35157614 | rs12964741 | A | 0.667 | 10489 | 2.06E−03 | 1.14 | 0.695 | 1671 | 3.40E−04 | 1.15 | 0.698 | 2002 |
| C08 | 73289641 | rs10504537 | A | 0.879 | 10635 | 2.08E−03 | 1.22 | 0.899 | 1700 | 4.08E−04 | 1.23 | 0.9 | 2034 |
| C06 | 72275474 | rs12207816 | A | 0.914 | 10635 | 2.08E−03 | 1.26 | 0.93 | 1700 | 7.95E−04 | 1.26 | 0.93 | 2034 |
| C19 | 4202069 | rs1045750 | C | 0.265 | 10634 | 2.08E−03 | 1.14 | 0.292 | 1699 | 6.95E−03 | 1.12 | 0.287 | 2033 |
| C02 | 203864322 | rs6704822 | G | 0.912 | 10558 | 2.08E−03 | 1.26 | 0.929 | 1689 | 3.09E−03 | 1.22 | 0.927 | 2019 |
| C01 | 173423432 | rs2175177 | A | 0.465 | 10625 | 2.09E−03 | 1.13 | 0.495 | 1698 | 1.84E−03 | 1.12 | 0.493 | 2031 |
| CX | 115198464 | rs2526763 | G | 0.365 | 8266 | 2.09E−03 | 1.17 | 0.401 | 1102 | 7.05E−03 | 1.13 | 0.394 | 1315 |
| C11 | 134180799 | rs1289455 | T | 0.147 | 10628 | 2.09E−03 | 1.18 | 0.169 | 1698 | 2.49E−03 | 1.16 | 0.167 | 2032 |
| C01 | 238623817 | rs3014559 | A | 0.546 | 10632 | 2.10E−03 | 1.13 | 0.576 | 1700 | 2.98E−03 | 1.12 | 0.573 | 2034 |
| C02 | 76254681 | rs317285 | A | 0.504 | 10634 | 2.10E−03 | 1.13 | 0.534 | 1700 | 2.98E−03 | 1.11 | 0.531 | 2034 |
| C08 | 37465414 | rs7017907 | C | 0.083 | 10598 | 2.10E−03 | 1.23 | 0.101 | 1691 | 6.65E−03 | 1.19 | 0.098 | 2024 |
| C16 | 53597062 | rs3751812 | T | 0.404 | 10627 | 2.10E−03 | 1.13 | 0.433 | 1699 | 3.87E−04 | 1.14 | 0.436 | 2033 |
| C05 | 167526920 | rs1077046 | C | 0.553 | 10633 | 2.10E−03 | 1.13 | 0.583 | 1700 | 1.46E−02 | 1.09 | 0.575 | 2034 |
| C10 | 131748188 | rs11597160 | A | 0.906 | 10635 | 2.11E−03 | 1.25 | 0.923 | 1700 | 1.05E−02 | 1.18 | 0.919 | 2034 |
| C18 | 13442635 | rs12964045 | C | 0.13 | 10634 | 2.11E−03 | 1.19 | 0.151 | 1700 | 8.59E−03 | 1.15 | 0.147 | 2034 |
| C05 | 9694366 | rs3213627 | T | 0.167 | 10630 | 2.13E−03 | 1.17 | 0.19 | 1700 | 4.17E−03 | 1.15 | 0.187 | 2034 |
| C21 | 24693776 | rs2829040 | A | 0.449 | 10633 | 2.13E−03 | 1.13 | 0.479 | 1700 | 1.51E−03 | 1.12 | 0.478 | 2034 |
| C07 | 85269850 | rs1012995 | C | 0.219 | 10600 | 2.13E−03 | 1.15 | 0.244 | 1685 | 1.45E−03 | 1.15 | 0.243 | 2019 |
| C07 | 18443601 | rs2269752 | C | 0.93 | 10630 | 2.13E−03 | 1.29 | 0.944 | 1700 | 8.19E−03 | 1.22 | 0.941 | 2034 |
| C01 | 85995056 | rs1360903 | G | 0.511 | 10626 | 2.14E−03 | 1.13 | 0.541 | 1700 | 5.66E−03 | 1.11 | 0.536 | 2033 |
| C16 | 62628004 | rs2962080 | G | 0.609 | 10631 | 2.15E−03 | 1.13 | 0.638 | 1699 | 2.28E−03 | 1.12 | 0.636 | 2033 |
| C12 | 20081535 | rs2417821 | T | 0.749 | 10635 | 2.15E−03 | 1.15 | 0.775 | 1700 | 1.80E−04 | 1.18 | 0.778 | 2034 |
| C04 | 10634963 | rs2170752 | T | 0.576 | 10634 | 2.16E−03 | 1.13 | 0.606 | 1700 | 2.79E−02 | 1.09 | 0.596 | 2034 |
| C09 | 110310944 | rs4979085 | T | 0.297 | 10629 | 2.16E−03 | 1.14 | 0.324 | 1700 | 2.97E−03 | 1.12 | 0.322 | 2034 |
| C01 | 216145811 | rs11118175 | G | 0.076 | 10635 | 2.16E−03 | 1.24 | 0.092 | 1700 | 2.03E−03 | 1.22 | 0.091 | 2034 |
| C06 | 57080845 | rs7756421 | A | 0.84 | 10188 | 2.16E−03 | 1.19 | 0.862 | 1690 | 4.45E−03 | 1.16 | 0.859 | 2019 |
| C06 | 29594220 | rs3094564 | G | 0.386 | 10635 | 2.17E−03 | 1.13 | 0.415 | 1700 | 4.26E−03 | 1.11 | 0.411 | 2034 |
| C18 | 53282820 | rs580647 | A | 0.886 | 10622 | 2.17E−03 | 1.22 | 0.904 | 1698 | 6.59E−03 | 1.18 | 0.901 | 2031 |
| C14 | 63874253 | rs7145500 | A | 0.661 | 10634 | 2.17E−03 | 1.14 | 0.69 | 1700 | 1.37E−03 | 1.13 | 0.689 | 2034 |
| C02 | 218495194 | rs1478581 | G | 0.819 | 10632 | 2.18E−03 | 1.18 | 0.841 | 1700 | 3.13E−03 | 1.15 | 0.839 | 2032 |
| C13 | 102932541 | rs1347329 | C | 0.592 | 10616 | 2.18E−03 | 1.13 | 0.621 | 1700 | 1.53E−02 | 1.09 | 0.614 | 2033 |
| C05 | 39159439 | rs444556 | C | 0.301 | 10632 | 2.18E−03 | 1.14 | 0.329 | 1700 | 3.08E−03 | 1.12 | 0.326 | 2034 |
| C09 | 14600866 | rs1343705 | G | 0.471 | 10631 | 2.18E−03 | 1.13 | 0.501 | 1699 | 3.38E−03 | 1.11 | 0.498 | 2033 |
| C16 | 19038467 | rs8053779 | G | 0.734 | 10634 | 2.19E−03 | 1.15 | 0.76 | 1700 | 1.04E−02 | 1.11 | 0.754 | 2034 |
| C21 | 26972748 | rs2830357 | G | 0.568 | 10634 | 2.19E−03 | 1.13 | 0.598 | 1700 | 1.78E−03 | 1.12 | 0.597 | 2034 |
| C07 | 77798006 | rs2192658 | T | 0.076 | 10630 | 2.19E−03 | 1.24 | 0.092 | 1699 | 1.17E−02 | 1.18 | 0.088 | 2032 |
| C17 | 62590633 | rs1985961 | C | 0.412 | 10633 | 2.20E−03 | 1.13 | 0.442 | 1700 | 2.23E−03 | 1.12 | 0.44 | 2034 |
| C10 | 96398926 | rs10509680 | T | 0.05 | 10601 | 2.20E−03 | 1.29 | 0.064 | 1695 | 9.38E−03 | 1.23 | 0.061 | 2027 |
| C11 | 98634196 | rs1461684 | T | 0.849 | 10635 | 2.20E−03 | 1.19 | 0.87 | 1700 | 8.48E−03 | 1.15 | 0.866 | 2034 |
| C06 | 106231995 | rs4440505 | C | 0.781 | 10635 | 2.20E−03 | 1.16 | 0.806 | 1700 | 5.16E−03 | 1.13 | 0.802 | 2034 |
| C03 | 14269176 | rs2128163 | G | 0.533 | 10617 | 2.21E−03 | 1.13 | 0.563 | 1700 | 2.43E−03 | 1.12 | 0.56 | 2034 |
| C07 | 8516487 | rs12234501 | T | 0.133 | 10635 | 2.22E−03 | 1.19 | 0.154 | 1700 | 6.54E−04 | 1.19 | 0.155 | 2034 |
| C10 | 84892441 | rs10886432 | G | 0.638 | 10634 | 2.22E−03 | 1.14 | 0.666 | 1700 | 1.76E−04 | 1.16 | 0.67 | 2034 |
| C08 | 41405289 | rs7819949 | T | 0.271 | 10634 | 2.23E−03 | 1.14 | 0.299 | 1700 | 4.46E−02 | 1.08 | 0.288 | 2034 |
| C21 | 42743171 | rs3788014 | C | 0.925 | 10631 | 2.23E−03 | 1.28 | 0.94 | 1700 | 2.98E−03 | 1.24 | 0.939 | 2033 |
| C16 | 75032079 | rs4243110 | T | 0.458 | 10628 | 2.23E−03 | 1.13 | 0.489 | 1699 | 4.24E−03 | 1.11 | 0.485 | 2033 |
| C15 | 52604593 | rs2414325 | T | 0.489 | 10634 | 2.23E−03 | 1.13 | 0.519 | 1700 | 1.35E−03 | 1.12 | 0.518 | 2034 |
| C07 | 86657613 | rs31662 | G | 0.862 | 10635 | 2.24E−03 | 1.2 | 0.882 | 1700 | 4.06E−03 | 1.17 | 0.88 | 2034 |
| C01 | 148824678 | rs6702842 | G | 0.269 | 10611 | 2.25E−03 | 1.14 | 0.296 | 1694 | 1.24E−02 | 1.11 | 0.289 | 2028 |
| C06 | 28141066 | rs149951 | T | 0.726 | 10635 | 2.25E−03 | 1.15 | 0.753 | 1700 | 2.41E−02 | 1.1 | 0.744 | 2034 |
| C06 | 28335583 | rs1635 | G | 0.942 | 10565 | 2.25E−03 | 1.32 | 0.955 | 1689 | 8.32E−03 | 1.24 | 0.953 | 2019 |
| C16 | 53594877 | rs8050136 | A | 0.404 | 10634 | 2.26E−03 | 1.13 | 0.433 | 1699 | 4.22E−04 | 1.14 | 0.436 | 2033 |
| C19 | 48943265 | rs346535 | T | 0.234 | 10635 | 2.26E−03 | 1.15 | 0.26 | 1700 | 4.17E−03 | 1.13 | 0.257 | 2034 |
| C13 | 101270990 | rs2038709 | C | 0.081 | 10629 | 2.26E−03 | 1.23 | 0.098 | 1700 | 1.17E−02 | 1.18 | 0.094 | 2034 |
| C01 | 19425721 | rs3748744 | G | 0.899 | 10634 | 2.26E−03 | 1.23 | 0.916 | 1700 | 2.68E−02 | 1.15 | 0.911 | 2034 |
| C04 | 107037574 | rs1982346 | C | 0.062 | 10480 | 2.27E−03 | 1.27 | 0.077 | 1682 | 1.13E−03 | 1.26 | 0.077 | 2011 |
| C15 | 34830800 | rs1194620 | T | 0.232 | 10633 | 2.27E−03 | 1.15 | 0.257 | 1700 | 9.03E−03 | 1.12 | 0.252 | 2034 |
| C10 | 5948421 | rs7912210 | C | 0.963 | 10616 | 2.28E−03 | 1.42 | 0.973 | 1679 | 5.95E−04 | 1.45 | 0.974 | 2012 |
| C21 | 14334270 | rs2822391 | A | 0.548 | 10634 | 2.28E−03 | 1.13 | 0.578 | 1700 | 8.05E−04 | 1.13 | 0.578 | 2034 |
| C05 | 15952862 | rs7715223 | C | 0.819 | 10597 | 2.28E−03 | 1.18 | 0.842 | 1692 | 1.73E−02 | 1.12 | 0.835 | 2026 |
| C02 | 75385815 | rs4853116 | A | 0.624 | 10364 | 2.29E−03 | 1.13 | 0.653 | 1695 | 2.15E−03 | 1.12 | 0.651 | 2025 |
| C05 | 56050092 | rs4700485 | A | 0.322 | 10635 | 2.29E−03 | 1.14 | 0.351 | 1700 | 1.00E−02 | 1.1 | 0.344 | 2034 |
| C01 | 184820295 | rs2383658 | G | 0.388 | 10633 | 2.29E−03 | 1.13 | 0.417 | 1700 | 1.58E−03 | 1.12 | 0.416 | 2034 |
| C03 | 82844262 | rs13075335 | T | 0.9 | 10058 | 2.29E−03 | 1.24 | 0.918 | 1678 | 9.58E−03 | 1.18 | 0.914 | 2004 |
| C04 | 82322766 | rs6830629 | A | 0.975 | 10625 | 2.30E−03 | 1.56 | 0.984 | 1699 | 8.03E−03 | 1.41 | 0.983 | 2032 |
| C14 | 63868436 | rs2411822 | C | 0.485 | 10630 | 2.30E−03 | 1.13 | 0.515 | 1700 | 9.77E−03 | 1.1 | 0.509 | 2034 |
| C05 | 55844670 | rs30365 | C | 0.898 | 10635 | 2.30E−03 | 1.23 | 0.915 | 1700 | 5.89E−03 | 1.19 | 0.912 | 2034 |
| C06 | 29516393 | rs2074464 | T | 0.445 | 10625 | 2.31E−03 | 1.13 | 0.475 | 1699 | 9.91E−03 | 1.1 | 0.469 | 2033 |
| C06 | 29588861 | rs3094572 | A | 0.386 | 10635 | 2.31E−03 | 1.13 | 0.416 | 1700 | 4.61E−03 | 1.11 | 0.412 | 2034 |
| C01 | 218026319 | rs2642438 | G | 0.71 | 10581 | 2.31E−03 | 1.14 | 0.737 | 1693 | 1.21E−02 | 1.11 | 0.731 | 2025 |
| C14 | 101389489 | rs4708 | A | 0.546 | 10627 | 2.31E−03 | 1.13 | 0.576 | 1698 | 4.92E−03 | 1.11 | 0.572 | 2030 |
| C02 | 76278450 | rs404892 | C | 0.505 | 10634 | 2.31E−03 | 1.13 | 0.535 | 1700 | 3.19E−03 | 1.11 | 0.532 | 2034 |
| C11 | 1341537 | rs7122936 | A | 0.572 | 10556 | 2.31E−03 | 1.13 | 0.602 | 1700 | 4.84E−03 | 1.11 | 0.598 | 2034 |
| C11 | 1341537 | rs7122936 | A | 0.572 | 10556 | 2.31E−03 | 1.13 | 0.602 | 1700 | 4.84E−03 | 1.11 | 0.598 | 2034 |
| C05 | 56080018 | rs12657615 | C | 0.115 | 10635 | 2.32E−03 | 1.2 | 0.135 | 1700 | 5.17E−03 | 1.17 | 0.132 | 2034 |
| C14 | 64537666 | rs6573648 | G | 0.274 | 10634 | 2.33E−03 | 1.14 | 0.301 | 1700 | 2.25E−03 | 1.13 | 0.299 | 2034 |
| C17 | 70794100 | rs2159010 | A | 0.727 | 10583 | 2.33E−03 | 1.15 | 0.753 | 1700 | 4.40E−03 | 1.13 | 0.75 | 2032 |
| C07 | 46518795 | rs956010 | A | 0.88 | 10635 | 2.33E−03 | 1.21 | 0.899 | 1700 | 2.09E−03 | 1.2 | 0.898 | 2034 |
| C15 | 92040939 | rs10152962 | G | 0.154 | 10635 | 2.33E−03 | 1.17 | 0.176 | 1700 | 7.80E−04 | 1.18 | 0.177 | 2034 |
| C20 | 49591938 | rs913670 | C | 0.631 | 10595 | 2.33E−03 | 1.13 | 0.66 | 1700 | 3.99E−03 | 1.12 | 0.656 | 2033 |
| C20 | 57938553 | rs6128397 | G | 0.689 | 10633 | 2.34E−03 | 1.14 | 0.716 | 1700 | 7.46E−03 | 1.11 | 0.711 | 2034 |
| C21 | 21803122 | rs2017705 | C | 0.073 | 10598 | 2.34E−03 | 1.24 | 0.089 | 1696 | 1.14E−02 | 1.19 | 0.086 | 2029 |
| C05 | 3996863 | rs634344 | G | 0.067 | 10633 | 2.34E−03 | 1.25 | 0.082 | 1700 | 6.05E−03 | 1.21 | 0.079 | 2033 |
| C06 | 27418512 | rs9461366 | G | 0.614 | 10635 | 2.34E−03 | 1.13 | 0.643 | 1700 | 6.05E−03 | 1.11 | 0.638 | 2034 |
| C22 | 19648862 | rs178260 | T | 0.14 | 10632 | 2.34E−03 | 1.18 | 0.161 | 1700 | 6.21E−03 | 1.15 | 0.158 | 2034 |
| C09 | 112545345 | rs2236388 | G | 0.899 | 10634 | 2.35E−03 | 1.23 | 0.917 | 1700 | 7.73E−04 | 1.24 | 0.917 | 2034 |
| C07 | 19730452 | rs10486378 | G | 0.277 | 10449 | 2.35E−03 | 1.14 | 0.304 | 1673 | 1.11E−02 | 1.11 | 0.298 | 2002 |
| C05 | 6325742 | rs1457122 | T | 0.333 | 10633 | 2.35E−03 | 1.13 | 0.361 | 1700 | 2.33E−03 | 1.12 | 0.359 | 2034 |
| C08 | 92473760 | rs2607102 | T | 0.927 | 10586 | 2.35E−03 | 1.28 | 0.942 | 1690 | 5.93E−03 | 1.23 | 0.939 | 2020 |
| C06 | 165937562 | rs9348022 | T | 0.657 | 10631 | 2.37E−03 | 1.14 | 0.685 | 1699 | 5.08E−03 | 1.12 | 0.681 | 2033 |
| C10 | 78039047 | rs1865017 | C | 0.734 | 10633 | 2.37E−03 | 1.15 | 0.76 | 1700 | 5.34E−03 | 1.12 | 0.756 | 2034 |
| C13 | 45593565 | rs9534568 | A | 0.76 | 10269 | 2.37E−03 | 1.15 | 0.785 | 1695 | 2.46E−03 | 1.14 | 0.783 | 2024 |
| C08 | 32774151 | rs9656744 | T | 0.511 | 10632 | 2.38E−03 | 1.13 | 0.541 | 1699 | 3.71E−03 | 1.11 | 0.537 | 2030 |
| C09 | 68331465 | rs943535 | A | 0.917 | 10584 | 2.39E−03 | 1.26 | 0.933 | 1690 | 9.80E−04 | 1.26 | 0.933 | 2020 |
| C10 | 87769103 | rs10749535 | T | 0.472 | 10635 | 2.40E−03 | 1.13 | 0.502 | 1700 | 2.92E−03 | 1.11 | 0.499 | 2034 |
| C11 | 98579265 | rs7120353 | T | 0.899 | 10634 | 2.40E−03 | 1.23 | 0.916 | 1700 | 2.08E−03 | 1.22 | 0.915 | 2034 |
| C05 | 31377276 | rs6875512 | A | 0.742 | 10632 | 2.40E−03 | 1.15 | 0.768 | 1700 | 7.97E−03 | 1.12 | 0.763 | 2034 |
| C02 | 198322740 | rs6434921 | A | 0.392 | 10635 | 2.40E−03 | 1.13 | 0.421 | 1700 | 1.59E−02 | 1.09 | 0.414 | 2034 |
| C06 | 5382731 | rs2113691 | C | 0.488 | 10479 | 2.40E−03 | 1.13 | 0.518 | 1644 | 3.11E−03 | 1.12 | 0.515 | 1974 |
| C10 | 50087691 | rs3793786 | C | 0.24 | 10635 | 2.40E−03 | 1.15 | 0.266 | 1700 | 4.64E−03 | 1.13 | 0.262 | 2034 |
| C08 | 23073423 | rs11775256 | T | 0.27 | 10634 | 2.41E−03 | 1.14 | 0.297 | 1700 | 9.12E−03 | 1.11 | 0.292 | 2034 |
| C12 | 116686949 | rs2592289 | A | 0.156 | 10632 | 2.41E−03 | 1.17 | 0.179 | 1700 | 2.57E−03 | 1.16 | 0.177 | 2034 |
| C07 | 157067324 | rs11769878 | C | 0.742 | 10433 | 2.41E−03 | 1.15 | 0.768 | 1690 | 2.84E−03 | 1.14 | 0.766 | 2022 |
| C11 | 56669965 | rs717117 | G | 0.075 | 10632 | 2.41E−03 | 1.24 | 0.091 | 1700 | 7.09E−03 | 1.19 | 0.088 | 2034 |
| C21 | 14824849 | rs2822753 | G | 0.357 | 10631 | 2.41E−03 | 1.13 | 0.386 | 1700 | 4.57E−03 | 1.11 | 0.382 | 2034 |
| C16 | 53995959 | rs1033043 | T | 0.574 | 10635 | 2.41E−03 | 1.13 | 0.604 | 1700 | 1.67E−02 | 1.09 | 0.596 | 2034 |
| C09 | 33091838 | rs10758186 | G | 0.381 | 10633 | 2.41E−03 | 1.13 | 0.41 | 1700 | 5.48E−03 | 1.11 | 0.406 | 2034 |
| C08 | 125394399 | rs4620270 | T | 0.13 | 10627 | 2.42E−03 | 1.19 | 0.15 | 1699 | 8.40E−04 | 1.19 | 0.151 | 2033 |
| C01 | 69518464 | rs10489554 | T | 0.77 | 10633 | 2.42E−03 | 1.16 | 0.795 | 1700 | 4.45E−03 | 1.13 | 0.792 | 2034 |
| C01 | 153811448 | rs12567958 | C | 0.266 | 10615 | 2.42E−03 | 1.14 | 0.293 | 1700 | 7.12E−03 | 1.12 | 0.288 | 2034 |
| C04 | 17086471 | rs7442366 | T | 0.821 | 10635 | 2.42E−03 | 1.17 | 0.844 | 1700 | 4.54E−04 | 1.19 | 0.845 | 2034 |
| C07 | 99312573 | rs999885 | T | 0.498 | 10467 | 2.43E−03 | 1.13 | 0.528 | 1678 | 4.30E−03 | 1.11 | 0.524 | 2012 |
| C03 | 61747598 | rs603052 | T | 0.778 | 10629 | 2.43E−03 | 1.16 | 0.802 | 1700 | 4.06E−03 | 1.14 | 0.799 | 2034 |
| C07 | 72804700 | rs7787362 | C | 0.557 | 10628 | 2.43E−03 | 1.13 | 0.587 | 1700 | 1.23E−02 | 1.1 | 0.58 | 2034 |
| C06 | 79723924 | rs1338023 | T | 0.623 | 10465 | 2.43E−03 | 1.13 | 0.652 | 1647 | 3.54E−03 | 1.12 | 0.649 | 1975 |
| C05 | 667701 | rs1697952 | C | 0.368 | 10612 | 2.43E−03 | 1.13 | 0.397 | 1697 | 4.73E−03 | 1.11 | 0.393 | 2030 |
| C17 | 40648038 | rs1122326 | C | 0.241 | 10633 | 2.44E−03 | 1.15 | 0.267 | 1700 | 3.93E−03 | 1.13 | 0.264 | 2034 |
| C10 | 10412081 | rs10508403 | T | 0.091 | 10635 | 2.44E−03 | 1.22 | 0.109 | 1700 | 2.93E−02 | 1.14 | 0.103 | 2034 |
| C02 | 171512196 | rs1837241 | A | 0.697 | 10634 | 2.44E−03 | 1.14 | 0.724 | 1700 | 1.28E−03 | 1.14 | 0.724 | 2034 |
| C06 | 17345030 | rs6940891 | C | 0.169 | 10635 | 2.45E−03 | 1.17 | 0.192 | 1700 | 2.87E−03 | 1.15 | 0.19 | 2034 |
| C06 | 42287224 | rs713383 | G | 0.684 | 10635 | 2.45E−03 | 1.14 | 0.712 | 1700 | 4.07E−03 | 1.12 | 0.708 | 2034 |
| C12 | 121500993 | rs7979863 | A | 0.375 | 10630 | 2.46E−03 | 1.13 | 0.404 | 1700 | 2.05E−03 | 1.12 | 0.402 | 2034 |
| C06 | 37577310 | rs9380681 | C | 0.308 | 10635 | 2.46E−03 | 1.14 | 0.335 | 1700 | 2.37E−03 | 1.13 | 0.333 | 2034 |
| C17 | 77817713 | rs17674253 | A | 0.579 | 10632 | 2.46E−03 | 1.13 | 0.608 | 1698 | 7.85E−03 | 1.1 | 0.603 | 2032 |
| C01 | 113140463 | rs773586 | A | 0.483 | 10633 | 2.46E−03 | 1.13 | 0.513 | 1700 | 3.45E−04 | 1.14 | 0.515 | 2034 |
| C06 | 92200200 | rs6454860 | G | 0.325 | 10314 | 2.46E−03 | 1.14 | 0.353 | 1639 | 3.16E−03 | 1.12 | 0.35 | 1969 |
| C10 | 61460650 | rs4948410 | A | 0.784 | 10631 | 2.47E−03 | 1.16 | 0.808 | 1700 | 4.52E−03 | 1.14 | 0.805 | 2034 |
| C06 | 157865565 | rs16900254 | A | 0.408 | 10635 | 2.47E−03 | 1.13 | 0.437 | 1700 | 1.34E−02 | 1.1 | 0.43 | 2034 |
| C15 | 93466050 | rs176656 | A | 0.09 | 10635 | 2.48E−03 | 1.22 | 0.108 | 1700 | 6.94E−04 | 1.23 | 0.109 | 2034 |
| C11 | 102978426 | rs719527 | C | 0.233 | 10612 | 2.49E−03 | 1.15 | 0.258 | 1700 | 5.40E−03 | 1.13 | 0.254 | 2034 |
| C20 | 57627978 | rs6128327 | A | 0.64 | 10481 | 2.50E−03 | 1.13 | 0.668 | 1683 | 1.61E−03 | 1.13 | 0.667 | 2015 |
| C07 | 21751434 | rs1174943 | G | 0.155 | 10631 | 2.51E−03 | 1.17 | 0.176 | 1700 | 1.20E−02 | 1.13 | 0.171 | 2034 |
| C07 | 8617064 | rs917038 | C | 0.784 | 10591 | 2.51E−03 | 1.16 | 0.808 | 1689 | 2.80E−02 | 1.1 | 0.8 | 2023 |
| C20 | 22311552 | rs199812 | C | 0.886 | 10635 | 2.51E−03 | 1.22 | 0.904 | 1700 | 9.30E−04 | 1.22 | 0.904 | 2034 |
| C04 | 147576738 | rs2241521 | A | 0.234 | 10634 | 2.51E−03 | 1.15 | 0.26 | 1700 | 1.05E−02 | 1.11 | 0.254 | 2034 |
| C11 | 20014361 | rs920671 | A | 0.182 | 10634 | 2.51E−03 | 1.16 | 0.205 | 1700 | 2.83E−03 | 1.15 | 0.203 | 2034 |
| C11 | 82788837 | rs4623925 | A | 0.267 | 10633 | 2.51E−03 | 1.14 | 0.294 | 1700 | 2.34E−02 | 1.1 | 0.286 | 2034 |
| C21 | 24056599 | rs2828515 | G | 0.122 | 10633 | 2.52E−03 | 1.19 | 0.142 | 1700 | 8.56E−04 | 1.2 | 0.143 | 2034 |
| C02 | 44054859 | rs6709904 | A | 0.907 | 10635 | 2.52E−03 | 1.24 | 0.924 | 1700 | 1.38E−03 | 1.24 | 0.924 | 2034 |
| C17 | 21252111 | rs9911448 | C | 0.962 | 10635 | 2.52E−03 | 1.41 | 0.972 | 1700 | 5.90E−03 | 1.33 | 0.971 | 2034 |
| C18 | 55909735 | rs9948303 | G | 0.099 | 10635 | 2.52E−03 | 1.21 | 0.117 | 1700 | 1.15E−03 | 1.21 | 0.117 | 2034 |
| C01 | 20385176 | rs2072671 | C | 0.314 | 10635 | 2.53E−03 | 1.13 | 0.341 | 1700 | 8.33E−04 | 1.14 | 0.342 | 2034 |
| C01 | 113074488 | rs6688859 | T | 0.68 | 10635 | 2.53E−03 | 1.14 | 0.708 | 1700 | 2.92E−04 | 1.15 | 0.711 | 2034 |
| C15 | 95693327 | rs7177625 | G | 0.683 | 10633 | 2.53E−03 | 1.14 | 0.71 | 1700 | 9.07E−03 | 1.11 | 0.705 | 2033 |
| C12 | 20118148 | rs11045018 | G | 0.749 | 10626 | 2.53E−03 | 1.15 | 0.774 | 1699 | 5.07E−04 | 1.16 | 0.776 | 2033 |
| C16 | 82538999 | rs1870843 | T | 0.599 | 10633 | 2.53E−03 | 1.13 | 0.628 | 1700 | 2.99E−03 | 1.12 | 0.625 | 2034 |
| C01 | 78369472 | rs1543675 | C | 0.376 | 10633 | 2.53E−03 | 1.13 | 0.405 | 1700 | 1.08E−02 | 1.1 | 0.399 | 2034 |
| C01 | 107498557 | rs2335793 | C | 0.887 | 10628 | 2.53E−03 | 1.22 | 0.905 | 1698 | 8.13E−04 | 1.23 | 0.906 | 2031 |
| C03 | 6741178 | rs978658 | G | 0.611 | 10635 | 2.54E−03 | 1.13 | 0.639 | 1700 | 1.13E−03 | 1.13 | 0.639 | 2034 |
| C05 | 76843306 | rs690280 | T | 0.422 | 10580 | 2.54E−03 | 1.13 | 0.451 | 1694 | 8.75E−03 | 1.1 | 0.446 | 2028 |
| C10 | 30821283 | rs7086224 | A | 0.418 | 10633 | 2.54E−03 | 1.13 | 0.448 | 1700 | 4.77E−03 | 1.11 | 0.444 | 2034 |
| C07 | 86648971 | rs2373593 | A | 0.862 | 10635 | 2.55E−03 | 1.2 | 0.882 | 1700 | 4.51E−03 | 1.17 | 0.88 | 2034 |
| C19 | 53768499 | rs3760802 | A | 0.499 | 10633 | 2.55E−03 | 1.13 | 0.528 | 1699 | 3.62E−03 | 1.11 | 0.525 | 2033 |
| C17 | 40663717 | rs12603327 | C | 0.241 | 10632 | 2.55E−03 | 1.15 | 0.267 | 1700 | 4.05E−03 | 1.13 | 0.264 | 2034 |
| C10 | 101886011 | rs1539089 | C | 0.222 | 10635 | 2.55E−03 | 1.15 | 0.247 | 1700 | 1.13E−02 | 1.12 | 0.241 | 2034 |
| C19 | 8795907 | rs10406508 | T | 0.764 | 10635 | 2.56E−03 | 1.15 | 0.789 | 1700 | 2.84E−02 | 1.1 | 0.78 | 2034 |
| C05 | 50786999 | rs1423611 | C | 0.445 | 10631 | 2.56E−03 | 1.13 | 0.474 | 1700 | 5.58E−03 | 1.11 | 0.47 | 2034 |
| C05 | 50786999 | rs1423611 | C | 0.445 | 10631 | 2.56E−03 | 1.13 | 0.474 | 1700 | 5.58E−03 | 1.11 | 0.47 | 2034 |
| C06 | 29574824 | rs1345227 | G | 0.379 | 10630 | 2.57E−03 | 1.13 | 0.408 | 1700 | 4.62E−03 | 1.11 | 0.404 | 2034 |
| C02 | 172857641 | rs6758704 | T | 0.709 | 10633 | 2.57E−03 | 1.14 | 0.736 | 1700 | 4.05E−03 | 1.12 | 0.733 | 2034 |
| C05 | 9699750 | rs2106687 | G | 0.125 | 10631 | 2.58E−03 | 1.19 | 0.146 | 1700 | 6.01E−03 | 1.16 | 0.142 | 2033 |
| C22 | 19683076 | rs430305 | T | 0.828 | 10635 | 2.58E−03 | 1.18 | 0.85 | 1700 | 2.96E−03 | 1.16 | 0.848 | 2034 |
| C19 | 22695812 | rs4933027 | T | 0.085 | 10634 | 2.58E−03 | 1.22 | 0.102 | 1700 | 9.19E−03 | 1.18 | 0.099 | 2034 |
| C11 | 83108627 | rs7952084 | A | 0.357 | 10635 | 2.58E−03 | 1.13 | 0.386 | 1700 | 5.81E−03 | 1.11 | 0.382 | 2034 |
| C07 | 27992355 | rs10225945 | A | 0.846 | 10535 | 2.59E−03 | 1.19 | 0.867 | 1697 | 4.58E−03 | 1.16 | 0.865 | 2030 |
| C04 | 10063925 | rs714086 | G | 0.759 | 10631 | 2.59E−03 | 1.15 | 0.784 | 1700 | 1.36E−02 | 1.11 | 0.778 | 2033 |
| C08 | 138245904 | rs4451334 | A | 0.062 | 10632 | 2.59E−03 | 1.26 | 0.077 | 1698 | 7.62E−03 | 1.21 | 0.074 | 2032 |
| C05 | 115396824 | rs9326981 | C | 0.949 | 10634 | 2.59E−03 | 1.34 | 0.961 | 1700 | 3.01E−03 | 1.3 | 0.96 | 2034 |
| C10 | 101903540 | rs11190578 | T | 0.222 | 10631 | 2.59E−03 | 1.15 | 0.247 | 1700 | 1.15E−02 | 1.12 | 0.241 | 2034 |
| C10 | 102146771 | rs4630219 | C | 0.099 | 10572 | 2.60E−03 | 1.21 | 0.117 | 1696 | 2.90E−03 | 1.19 | 0.115 | 2027 |
| C17 | 40667190 | rs7214921 | C | 0.241 | 10634 | 2.60E−03 | 1.15 | 0.267 | 1700 | 4.13E−03 | 1.13 | 0.264 | 2034 |
| C14 | 64300426 | rs12433694 | C | 0.479 | 10635 | 2.61E−03 | 1.13 | 0.509 | 1700 | 1.43E−02 | 1.09 | 0.501 | 2034 |
| C22 | 18282773 | rs1978058 | T | 0.376 | 10630 | 2.61E−03 | 1.13 | 0.404 | 1700 | 1.08E−02 | 1.1 | 0.398 | 2033 |
| C04 | 131671262 | rs1027884 | T | 0.497 | 10634 | 2.61E−03 | 1.13 | 0.527 | 1700 | 2.40E−03 | 1.12 | 0.525 | 2034 |
| C08 | 28224848 | rs904053 | G | 0.225 | 10634 | 2.61E−03 | 1.15 | 0.25 | 1700 | 3.98E−02 | 1.09 | 0.24 | 2034 |
| C08 | 28224848 | rs904053 | G | 0.225 | 10634 | 2.61E−03 | 1.15 | 0.25 | 1700 | 3.98E−02 | 1.09 | 0.24 | 2034 |
| C16 | 54000987 | rs2665282 | A | 0.574 | 10607 | 2.61E−03 | 1.13 | 0.603 | 1696 | 1.79E−02 | 1.09 | 0.596 | 2030 |
| C05 | 136028429 | rs4141140 | G | 0.7 | 10632 | 2.62E−03 | 1.14 | 0.727 | 1700 | 1.58E−02 | 1.1 | 0.721 | 2034 |
| C07 | 49507399 | rs13240380 | T | 0.434 | 10632 | 2.62E−03 | 1.13 | 0.463 | 1700 | 1.78E−03 | 1.12 | 0.462 | 2033 |
| C09 | 11743695 | rs10960174 | G | 0.613 | 10629 | 2.62E−03 | 1.13 | 0.641 | 1698 | 1.74E−03 | 1.13 | 0.64 | 2031 |
| C15 | 43706836 | rs7172161 | G | 0.513 | 10634 | 2.62E−03 | 1.13 | 0.543 | 1700 | 2.59E−03 | 1.12 | 0.541 | 2034 |
| C04 | 162740637 | rs9942159 | T | 0.377 | 10550 | 2.63E−03 | 1.13 | 0.406 | 1691 | 2.09E−03 | 1.12 | 0.404 | 2024 |
| C05 | 158736740 | rs1433048 | G | 0.192 | 10317 | 2.63E−03 | 1.16 | 0.216 | 1652 | 1.82E−02 | 1.11 | 0.21 | 1981 |
| C10 | 84886235 | rs4411234 | C | 0.638 | 10631 | 2.63E−03 | 1.13 | 0.666 | 1700 | 2.14E−04 | 1.15 | 0.67 | 2034 |
| C12 | 100468481 | rs864302 | T | 0.483 | 10630 | 2.63E−03 | 1.13 | 0.512 | 1700 | 7.50E−03 | 1.1 | 0.507 | 2034 |
| C05 | 76657043 | rs251421 | C | 0.26 | 10618 | 2.64E−03 | 1.14 | 0.286 | 1690 | 5.58E−03 | 1.12 | 0.283 | 2023 |
| C07 | 46531242 | rs6956126 | G | 0.885 | 10632 | 2.64E−03 | 1.22 | 0.904 | 1700 | 2.41E−03 | 1.2 | 0.903 | 2034 |
| C09 | 129104520 | rs7026302 | G | 0.908 | 10633 | 2.64E−03 | 1.24 | 0.924 | 1700 | 2.87E−02 | 1.15 | 0.919 | 2034 |
| C11 | 121657794 | rs531743 | A | 0.954 | 10633 | 2.64E−03 | 1.36 | 0.966 | 1700 | 2.00E−03 | 1.34 | 0.965 | 2034 |
| C11 | 33462777 | rs10836066 | T | 0.301 | 10634 | 2.65E−03 | 1.14 | 0.328 | 1700 | 3.03E−02 | 1.09 | 0.319 | 2034 |
| C05 | 122595829 | rs9327287 | G | 0.262 | 10632 | 2.65E−03 | 1.14 | 0.288 | 1700 | 7.19E−03 | 1.12 | 0.284 | 2034 |
| C04 | 181363537 | rs17090640 | G | 0.952 | 10635 | 2.65E−03 | 1.35 | 0.964 | 1700 | 1.64E−02 | 1.24 | 0.961 | 2034 |
| C04 | 119551429 | rs6820367 | A | 0.35 | 10634 | 2.65E−03 | 1.13 | 0.379 | 1700 | 4.66E−03 | 1.11 | 0.375 | 2034 |
| C19 | 62070079 | rs8113561 | G | 0.954 | 10625 | 2.65E−03 | 1.36 | 0.966 | 1700 | 3.66E−02 | 1.21 | 0.962 | 2034 |
| C08 | 97162902 | rs2440213 | A | 0.358 | 10632 | 2.65E−03 | 1.13 | 0.387 | 1700 | 5.98E−03 | 1.11 | 0.383 | 2033 |
| C10 | 93013669 | rs1028935 | A | 0.185 | 10633 | 2.65E−03 | 1.16 | 0.208 | 1700 | 1.86E−03 | 1.15 | 0.207 | 2034 |
| C06 | 24562175 | rs9393558 | T | 0.347 | 10635 | 2.65E−03 | 1.13 | 0.375 | 1700 | 2.11E−02 | 1.09 | 0.367 | 2034 |
| C09 | 103242888 | rs7854755 | A | 0.198 | 10589 | 2.65E−03 | 1.16 | 0.222 | 1694 | 7.15E−03 | 1.13 | 0.218 | 2027 |
| C09 | 79420643 | rs10867740 | C | 0.236 | 10625 | 2.65E−03 | 1.15 | 0.262 | 1699 | 2.00E−03 | 1.14 | 0.261 | 2033 |
| C20 | 53952228 | rs6068930 | G | 0.039 | 10620 | 2.66E−03 | 1.32 | 0.051 | 1700 | 9.79E−03 | 1.26 | 0.049 | 2034 |
| C14 | 68370111 | rs1291302 | C | 0.259 | 10527 | 2.66E−03 | 1.14 | 0.285 | 1675 | 3.98E−03 | 1.13 | 0.282 | 2008 |
| C01 | 93943749 | rs4147861 | G | 0.923 | 10526 | 2.66E−03 | 1.27 | 0.939 | 1668 | 2.12E−03 | 1.25 | 0.938 | 2000 |
| C14 | 98178185 | rs2273840 | A | 0.931 | 10569 | 2.67E−03 | 1.28 | 0.945 | 1677 | 1.07E−03 | 1.29 | 0.945 | 2009 |
| C14 | 98178185 | rs2273840 | A | 0.931 | 10569 | 2.67E−03 | 1.28 | 0.945 | 1677 | 1.07E−03 | 1.29 | 0.945 | 2009 |
| CX | 138713457 | rs1948488 | A | 0.223 | 8219 | 2.68E−03 | 1.19 | 0.254 | 1094 | 4.52E−03 | 1.16 | 0.25 | 1307 |
| C12 | 20196656 | rs7299937 | T | 0.059 | 10635 | 2.68E−03 | 1.26 | 0.074 | 1700 | 7.12E−03 | 1.22 | 0.071 | 2034 |
| C04 | 87630493 | rs10516770 | T | 0.055 | 10635 | 2.69E−03 | 1.27 | 0.069 | 1700 | 4.44E−03 | 1.24 | 0.067 | 2034 |
| C02 | 172822314 | rs4668412 | A | 0.709 | 10634 | 2.69E−03 | 1.14 | 0.736 | 1700 | 4.24E−03 | 1.12 | 0.733 | 2034 |
| C06 | 36770559 | rs236444 | T | 0.253 | 10634 | 2.70E−03 | 1.14 | 0.279 | 1700 | 3.68E−04 | 1.16 | 0.281 | 2034 |
| C11 | 103253198 | rs10502020 | G | 0.446 | 10634 | 2.71E−03 | 1.13 | 0.476 | 1700 | 8.02E−03 | 1.1 | 0.47 | 2034 |
| C04 | 131653392 | rs4383635 | A | 0.491 | 10630 | 2.71E−03 | 1.13 | 0.521 | 1699 | 2.25E−03 | 1.12 | 0.519 | 2033 |
| C04 | 87557966 | rs2199309 | C | 0.229 | 10234 | 2.71E−03 | 1.15 | 0.255 | 1653 | 8.94E−04 | 1.15 | 0.256 | 1977 |
| C09 | 23737791 | rs1329044 | C | 0.153 | 10631 | 2.71E−03 | 1.17 | 0.175 | 1700 | 6.15E−04 | 1.18 | 0.176 | 2034 |
| C12 | 127797687 | rs470477 | T | 0.311 | 10632 | 2.71E−03 | 1.13 | 0.338 | 1700 | 5.14E−03 | 1.12 | 0.334 | 2033 |
| C13 | 105492839 | rs1927771 | A | 0.411 | 10633 | 2.72E−03 | 1.13 | 0.44 | 1700 | 4.66E−03 | 1.11 | 0.436 | 2034 |
| CX | 4916022 | rs6638525 | C | 0.107 | 8269 | 2.73E−03 | 1.25 | 0.13 | 1103 | 1.91E−03 | 1.24 | 0.129 | 1316 |
| C09 | 134921961 | rs2297538 | G | 0.73 | 10630 | 2.75E−03 | 1.15 | 0.756 | 11700 | 5.61E−03 | 1.12 | 0.752 | 2034 |
| C11 | 68645465 | rs2924536 | A | 0.447 | 10631 | 2.75E−03 | 1.13 | 0.476 | 1700 | 1.83E−02 | 1.09 | 0.469 | 2034 |
| C02 | 173022859 | rs10165126 | C | 0.705 | 10630 | 2.75E−03 | 1.14 | 0.732 | 1699 | 2.98E−03 | 1.13 | 0.729 | 2033 |
| C11 | 10906227 | rs10500734 | A | 0.767 | 10617 | 2.75E−03 | 1.15 | 0.791 | 1692 | 2.25E−02 | 1.11 | 0.784 | 2025 |
| C02 | 12183852 | rs10929777 | C | 0.3 | 10634 | 2.75E−03 | 1.14 | 0.327 | 1700 | 7.76E−03 | 1.11 | 0.323 | 2034 |
| C01 | 106549733 | rs10494048 | T | 0.387 | 10589 | 2.75E−03 | 1.13 | 0.416 | 1699 | 2.94E−02 | 1.08 | 0.406 | 2030 |
| C10 | 69228609 | rs17456902 | G | 0.872 | 10635 | 2.76E−03 | 1.2 | 0.891 | 1700 | 3.98E−03 | 1.18 | 0.889 | 2034 |
| C04 | 90099908 | rs2924936 | G | 0.869 | 10635 | 2.76E−03 | 1.2 | 0.888 | 1700 | 3.77E−04 | 1.22 | 0.89 | 2034 |
| C06 | 37561316 | rs9296225 | C | 0.472 | 10635 | 2.76E−03 | 1.13 | 0.502 | 1700 | 3.40E−03 | 1.11 | 0.499 | 2034 |
| C22 | 17337894 | rs2019061 | T | 0.685 | 10621 | 2.77E−03 | 1.14 | 0.712 | 1694 | 6.23E−03 | 1.12 | 0.708 | 2028 |
| C02 | 16792100 | rs11897395 | C | 0.889 | 10590 | 2.77E−03 | 1.22 | 0.907 | 1686 | 1.35E−02 | 1.16 | 0.903 | 2020 |
| C02 | 43918060 | rs1991739 | A | 0.619 | 10632 | 2.77E−03 | 1.13 | 0.647 | 1700 | 1.72E−02 | 1.09 | 0.64 | 2034 |
| C07 | 102594288 | rs7804867 | A | 0.205 | 10634 | 2.78E−03 | 1.15 | 0.229 | 1700 | 1.92E−03 | 1.15 | 0.228 | 2034 |
| C06 | 10170597 | rs6459613 | A | 0.545 | 10601 | 2.78E−03 | 1.13 | 0.574 | 1698 | 2.65E−02 | 1.09 | 0.565 | 2030 |
| C05 | 50904720 | rs7708362 | T | 0.345 | 10619 | 2.78E−03 | 1.13 | 0.373 | 1687 | 5.55E−03 | 1.11 | 0.369 | 2021 |
| C05 | 94682297 | rs464906 | C | 0.614 | 10630 | 2.78E−03 | 1.13 | 0.642 | 1700 | 9.79E−03 | 1.1 | 0.637 | 2034 |
| C10 | 8311160 | rs1419294 | T | 0.711 | 10635 | 2.78E−03 | 1.14 | 0.738 | 1700 | 1.09E−03 | 1.14 | 0.738 | 2034 |
| C10 | 2108862 | rs12776254 | G | 0.167 | 10634 | 2.78E−03 | 1.17 | 0.19 | 1700 | 1.95E−03 | 1.16 | 0.189 | 2034 |
| C20 | 59771295 | rs6128843 | T | 0.182 | 10634 | 2.79E−03 | 1.16 | 0.206 | 1700 | 1.16E−03 | 1.16 | 0.206 | 2034 |
| C02 | 242432072 | rs12052982 | A | 0.958 | 10630 | 2.79E−03 | 1.38 | 0.969 | 1698 | 7.24E−03 | 1.3 | 0.967 | 2032 |
| C10 | 102016372 | rs9420802 | G | 0.209 | 10632 | 2.80E−03 | 1.15 | 0.233 | 1698 | 1.15E−02 | 1.12 | 0.228 | 2032 |
| C07 | 149980541 | rs10277774 | C | 0.383 | 10632 | 2.80E−03 | 1.13 | 0.412 | 1699 | 1.84E−03 | 1.12 | 0.411 | 2033 |
| C12 | 21065184 | rs1910186 | C | 0.109 | 10630 | 2.80E−03 | 1.2 | 0.128 | 1700 | 1.84E−03 | 1.19 | 0.128 | 2034 |
| C12 | 21065184 | rs1910186 | C | 0.109 | 10630 | 2.80E−03 | 1.2 | 0.128 | 1700 | 1.84E−03 | 1.19 | 0.128 | 2034 |
| C17 | 73745676 | rs1000299 | A | 0.055 | 10635 | 2.81E−03 | 1.27 | 0.069 | 1700 | 2.50E−02 | 1.19 | 0.064 | 2034 |
| C06 | 162143621 | rs7739802 | T | 0.491 | 10594 | 2.81E−03 | 1.12 | 0.521 | 1699 | 4.70E−04 | 1.14 | 0.523 | 2030 |
| C11 | 102072274 | rs1276252 | G | 0.252 | 10634 | 2.81E−03 | 1.14 | 0.277 | 1700 | 1.08E−02 | 1.11 | 0.272 | 2034 |
| C12 | 123335210 | rs3759107 | T | 0.127 | 10635 | 2.81E−03 | 1.19 | 0.147 | 1700 | 3.67E−03 | 1.17 | 0.145 | 2034 |
| C11 | 42941748 | rs7939657 | T | 0.558 | 10632 | 2.81E−03 | 1.13 | 0.587 | 1700 | 3.56E−03 | 1.11 | 0.585 | 2034 |
| C14 | 63855666 | rs7147624 | G | 0.798 | 10635 | 2.81E−03 | 1.16 | 0.821 | 1700 | 3.48E−03 | 1.15 | 0.819 | 2034 |
| C07 | 21959042 | rs17146355 | A | 0.05 | 10619 | 2.82E−03 | 1.29 | 0.063 | 1697 | 1.93E−03 | 1.28 | 0.063 | 2031 |
| C01 | 7589173 | rs10462018 | C | 0.816 | 10632 | 2.82E−03 | 1.17 | 0.839 | 1700 | 1.67E−02 | 1.12 | 0.833 | 2034 |
| C15 | 87930503 | rs12595195 | C | 0.275 | 10632 | 2.82E−03 | 1.14 | 0.301 | 1700 | 5.15E−03 | 1.12 | 0.298 | 2034 |
| C04 | 90107239 | rs13130 | T | 0.87 | 10623 | 2.82E−03 | 1.2 | 0.889 | 1698 | 3.57E−04 | 1.22 | 0.891 | 2032 |
| C03 | 99664143 | rs1531377 | T | 0.173 | 10628 | 2.83E−03 | 1.16 | 0.196 | 1699 | 1.02E−02 | 1.13 | 0.191 | 2033 |
| C04 | 30136639 | rs2060515 | T | 0.111 | 10569 | 2.83E−03 | 1.2 | 0.13 | 1689 | 4.85E−03 | 1.17 | 0.127 | 2020 |
| C18 | 58447316 | rs2332575 | A | 0.053 | 10634 | 2.84E−03 | 1.28 | 0.066 | 1699 | 2.85E−03 | 1.26 | 0.065 | 2033 |
| C06 | 12338790 | rs1028716 | T | 0.06 | 10632 | 2.84E−03 | 1.26 | 0.074 | 1700 | 4.77E−03 | 1.23 | 0.072 | 2034 |
| C05 | 55657524 | rs32496 | C | 0.573 | 10633 | 2.85E−03 | 1.13 | 0.601 | 1700 | 4.48E−03 | 1.11 | 0.598 | 2034 |
| C08 | 5835084 | rs890014 | C | 0.757 | 10635 | 2.86E−03 | 1.15 | 0.781 | 1700 | 4.61E−03 | 1.13 | 0.779 | 2034 |
| C21 | 14821123 | rs2822744 | A | 0.157 | 10633 | 2.86E−03 | 1.17 | 0.179 | 1700 | 4.96E−03 | 1.15 | 0.176 | 2034 |
| C06 | 19958854 | rs6905466 | T | 0.327 | 10635 | 2.86E−03 | 1.13 | 0.355 | 1700 | 1.18E−03 | 1.13 | 0.355 | 2034 |
| C15 | 77441636 | rs4255747 | G | 0.668 | 10635 | 2.86E−03 | 1.13 | 0.695 | 1700 | 1.01E−02 | 1.11 | 0.69 | 2034 |
| C15 | 53381369 | rs8041044 | C | 0.914 | 10627 | 2.86E−03 | 1.25 | 0.93 | 1698 | 1.09E−02 | 1.19 | 0.927 | 2032 |
| C02 | 24312396 | rs10200481 | A | 0.106 | 10635 | 2.86E−03 | 1.2 | 0.125 | 1700 | 1.22E−02 | 1.15 | 0.121 | 2033 |
| C10 | 16380276 | rs2184383 | G | 0.805 | 10628 | 2.87E−03 | 1.16 | 0.828 | 1700 | 4.18E−03 | 1.14 | 0.825 | 2034 |
| C09 | 8506194 | rs10119236 | A | 0.294 | 10632 | 2.87E−03 | 1.14 | 0.321 | 1700 | 2.66E−02 | 1.09 | 0.312 | 2034 |
| C02 | 78341614 | rs2861264 | T | 0.105 | 10635 | 2.87E−03 | 1.2 | 0.123 | 1700 | 3.16E−03 | 1.18 | 0.122 | 2034 |
| C18 | 58394216 | rs3744927 | G | 0.773 | 10634 | 2.88E−03 | 1.15 | 0.797 | 1700 | 1.35E−02 | 1.12 | 0.792 | 2034 |
| C02 | 77756026 | rs6547152 | C | 0.787 | 10620 | 2.89E−03 | 1.16 | 0.811 | 1698 | 1.38E−03 | 1.16 | 0.811 | 2032 |
| C12 | 52445647 | rs6580960 | A | 0.541 | 10635 | 2.89E−03 | 1.13 | 0.57 | 1700 | 3.40E−03 | 1.11 | 0.567 | 2034 |
| C07 | 27780647 | rs740127 | G | 0.595 | 10635 | 2.89E−03 | 1.13 | 0.624 | 1700 | 1.23E−02 | 1.1 | 0.618 | 2034 |
| C07 | 50816853 | rs17134122 | A | 0.966 | 10634 | 2.89E−03 | 1.43 | 0.976 | 1700 | 1.10E−02 | 1.32 | 0.974 | 2034 |
| C22 | 34868465 | rs132717 | C | 0.62 | 10359 | 2.90E−03 | 1.13 | 0.648 | 1664 | 2.36E−02 | 1.09 | 0.64 | 1990 |
| C18 | 55211067 | rs8088514 | C | 0.461 | 10602 | 2.90E−03 | 1.12 | 0.49 | 1693 | 1.65E−02 | 1.09 | 0.482 | 2027 |
| C10 | 119681455 | rs7922488 | G | 0.533 | 10616 | 2.90E−03 | 1.13 | 0.562 | 1692 | 4.75E−03 | 1.11 | 0.558 | 2026 |
| C05 | 158745072 | rs7709212 | T | 0.678 | 10635 | 2.90E−03 | 1.14 | 0.705 | 1700 | 1.55E−02 | 1.1 | 0.699 | 2034 |
| C05 | 23761851 | rs11743379 | T | 0.507 | 10633 | 2.91E−03 | 1.12 | 0.536 | 1700 | 4.46E−04 | 1.14 | 0.539 | 2034 |
| C06 | 130039640 | rs376632 | G | 0.89 | 10633 | 2.91E−03 | 1.22 | 0.908 | 1699 | 2.60E−03 | 1.2 | 0.907 | 2033 |
| C10 | 8277669 | rs4747760 | G | 0.875 | 10631 | 2.91E−03 | 1.2 | 0.894 | 1700 | 3.50E−03 | 1.18 | 0.892 | 2034 |
| C03 | 145966921 | rs13061557 | T | 0.699 | 10630 | 2.91E−03 | 1.14 | 0.726 | 1700 | 1.57E−02 | 1.1 | 0.719 | 2034 |
| C06 | 122997014 | rs9388117 | A | 0.671 | 10616 | 2.92E−03 | 1.13 | 0.698 | 1699 | 1.83E−02 | 1.1 | 0.691 | 2033 |
| C10 | 132443494 | rs893504 | C | 0.071 | 10635 | 2.92E−03 | 1.24 | 0.087 | 1700 | 2.64E−03 | 1.22 | 0.086 | 2034 |
| C04 | 40312795 | rs4447895 | C | 0.888 | 10634 | 2.92E−03 | 1.22 | 0.906 | 1700 | 1.56E−03 | 1.21 | 0.906 | 2034 |
| C06 | 162147237 | rs3016536 | G | 0.517 | 10635 | 2.93E−03 | 1.12 | 0.546 | 1700 | 1.72E−03 | 1.12 | 0.545 | 2034 |
| C15 | 58046702 | rs4775234 | C | 0.259 | 10530 | 2.93E−03 | 1.14 | 0.285 | 1681 | 3.63E−02 | 1.09 | 0.276 | 2009 |
| C10 | 107117270 | rs11192557 | T | 0.876 | 10623 | 2.93E−03 | 1.2 | 0.895 | 1698 | 2.56E−03 | 1.19 | 0.893 | 2032 |
| C07 | 1681599 | rs10267593 | G | 0.835 | 10635 | 2.93E−03 | 1.18 | 0.856 | 1700 | 2.02E−04 | 1.21 | 0.859 | 2034 |
| C14 | 98123983 | rs2895811 | C | 0.448 | 10607 | 3.04E−03 | 1.12 | 0.478 | 1694 | 3.73E−03 | 1.11 | 0.475 | 2027 |
| C17 | 18260758 | rs854771 | C | 0.554 | 10635 | 3.18E−03 | 1.12 | 0.583 | 1700 | 1.54E−03 | 1.12 | 0.583 | 2034 |
| C04 | 129890119 | rs4549394 | T | 0.097 | 10631 | 3.31E−03 | 1.21 | 0.115 | 1700 | 2.25E−03 | 1.2 | 0.114 | 2034 |
| C02 | 176994186 | rs12621733 | G | 0.899 | 10634 | 3.47E−03 | 1.22 | 0.916 | 1700 | 9.37E−04 | 1.24 | 0.917 | 2034 |
| C01 | 113692475 | rs1217407 | A | 0.249 | 10631 | 3.56E−03 | 1.14 | 0.274 | 1700 | 1.20E−03 | 1.14 | 0.275 | 2034 |
| C02 | 36037410 | rs10203034 | A | 0.68 | 10634 | 3.64E−03 | 1.13 | 0.706 | 1700 | 2.27E−03 | 1.13 | 0.706 | 2034 |
| C01 | 113628929 | rs2797409 | C | 0.249 | 10634 | 3.79E−03 | 1.14 | 0.274 | 1700 | 1.29E−03 | 1.14 | 0.275 | 2034 |
| C12 | 69325443 | rs7978668 | T | 0.918 | 10635 | 3.84E−03 | 1.25 | 0.933 | 1700 | 1.61E−03 | 1.25 | 0.933 | 2034 |
| C12 | 69326403 | rs767760 | A | 0.914 | 10635 | 3.96E−03 | 1.24 | 0.93 | 1700 | 1.10E−03 | 1.25 | 0.93 | 2034 |
| C12 | 69368222 | rs949664 | A | 0.863 | 10632 | 3.98E−03 | 1.19 | 0.882 | 1700 | 1.46E−03 | 1.19 | 0.882 | 2034 |
| C12 | 69340682 | rs7309888 | C | 0.794 | 10631 | 4.11E−03 | 1.15 | 0.816 | 1700 | 1.40E−03 | 1.16 | 0.817 | 2034 |
| C14 | 98160936 | rs12435918 | G | 0.333 | 10635 | 4.43E−03 | 1.12 | 0.36 | 1700 | 2.64E−03 | 1.12 | 0.359 | 2034 |
| C07 | 11669876 | rs2355129 | A | 0.362 | 10635 | 4.59E−03 | 1.12 | 0.389 | 1699 | 1.13E−03 | 1.13 | 0.391 | 2033 |
| C08 | 129057022 | rs2648896 | C | 0.149 | 10633 | 5.75E−03 | 1.16 | 0.169 | 1700 | 2.49E−03 | 1.16 | 0.169 | 2034 |
| C12 | 69347673 | rs7976576 | T | 0.794 | 10635 | 6.31E−03 | 1.15 | 0.816 | 1700 | 2.22E−03 | 1.15 | 0.816 | 2034 |
| C07 | 11658292 | rs10215881 | C | 0.494 | 10630 | 8.18E−03 | 1.11 | 0.52 | 1699 | 1.88E−03 | 1.12 | 0.523 | 2033 |
| C12 | 124462726 | rs7310042 | G | 0.193 | 10634 | 8.35E−03 | 1.14 | 0.214 | 1700 | 2.55E−03 | 1.15 | 0.215 | 2034 |
| C07 | 11646149 | rs6460851 | G | 0.288 | 10624 | 8.43E−03 | 1.12 | 0.312 | 1698 | 1.88E−03 | 1.13 | 0.314 | 2031 |
| C02 | 128444077 | rs46627431 | T | 0.515 | 10622 | 1.30E−02 | 1.1 | 0.539 | 1700 | 2.49E−03 | 1.12 | 0.543 | 2034 |

TABLE 2
Association results for the Icelandic discovery cohorts, and the replication cohorts. The
columns in the table indicate (1) the cohort, (2) rs name of the marker in question,
(3) the chromosome, (4) position of the marker in NCBI Build 34, (5) p-value of the
association, (6) the Relative Risk of the association, (7) the number of MI patients,
(8) the frequency of the associating allele in patients, (9) the number of controls,
(10) the frequency of the associating allele in controls, (11) the associating allele.

| Cohort | rs-name | chr | position | p-value | RR | Naff | f (aff) | Ncon | f (con) | allele |
|---|---|---|---|---|---|---|---|---|---|---|
| SG01S1480 | | C01 | 57254228 | | | | | | | |
| Iceland discovery group | rs2991515 | | | 0.001045 | 1.14 | 1642 | 0.514 | 6868 | 0.480 | T |
| Iceland replication group | rs2991515 | | | 0.054380 | 1.13 | 560 | 0.507 | 3393 | 0.476 | T |
| Iceland All | rs2991515 | | | 0.000108 | 1.14 | 2202 | 0.512 | 10261 | 0.479 | T |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | | | | 0.054379 | 1.13 | 544 | | | | T |
| All groups | | | | 0.000108 | 1.14 | 2016 | | | | T |
| SG01S1340 | | C01 | 113676295 | | | | | | | |
| Iceland discovery group | rs2476601 | | | 0.000452 | 1.23 | 1632 | 0.156 | 6858 | 0.130 | A |
| Iceland replication group | rs2476601 | | | 0.001253 | 1.34 | 562 | 0.167 | 3393 | 0.130 | A |
| Iceland All | rs2476601 | | | 0.000003 | 1.26 | 2194 | 0.159 | 10251 | 0.130 | A |
| Philadelphia | | | | NA | | | | | | |
| Atlanta | | | | NA | | | | | | |
| Durham | rs2476601 | | | 0.407813 | 1.12 | 932 | 0.095 | 544 | 0.086 | A |
| All US groups | | | | 0.407813 | 1.1164 | 932 | | | | A |
| All replication groups | | | | 0.001741 | 1.2678 | 1478 | | | | A |
| All groups | | | | 0.000003 | 1.2404 | 2941 | | | | A |
| SG02S870 | | C02 | 36022756 | | | | | | | |
| Iceland discovery group | rs6747236 | | | 0.004309 | 1.14 | 1634 | 0.743 | 6863 | 0.717 | T |
| Iceland replication group | rs6747236 | | | 0.101400 | 1.13 | 544 | 0.741 | 3391 | 0.717 | T |
| Iceland All | rs6747236 | | | 0.000905 | 1.14 | 2178 | 0.742 | 10254 | 0.717 | T |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | | | | 0.101403 | 1.13 | 528 | | | | T |
| All groups | | | | 0.000905 | 1.14 | 1994 | | | | T |
| SG02S840 | | C02 | 176947426 | | | | | | | |
| Iceland discovery group | rs921083 | | | 0.000163 | 1.21 | 1642 | 0.806 | 6867 | 0.774 | G |
| Iceland replication group | rs921083 | | | 0.054180 | 1.17 | 549 | 0.805 | 3392 | 0.779 | G |
| Iceland All | rs921083 | | | 0.000025 | 1.20 | 2191 | 0.806 | 10259 | 0.776 | G |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | | | | 0.054178 | 1.17 | 533 | | | | G |
| All groups | | | | 0.000025 | 1.20 | 2006 | | | | G |
| SG02S832 | | C02 | 212588265 | | | | | | | |
| Iceland discovery group | rs12329252 | | | 0.000680 | 1.16 | 1641 | 0.710 | 6868 | 0.677 | T |
| Iceland replication group | rs12329252 | | | 0.327900 | 1.07 | 554 | 0.692 | 3393 | 0.677 | T |
| Iceland All | rs12329252 | | | 0.000500 | 1.14 | 2195 | 0.705 | 10261 | 0.677 | T |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | | | | 0.327938 | 1.07 | 538 | | | | T |
| All groups | | | | 0.000500 | 1.14 | 2010 | | | | T |
| SG03S670 | | C03 | 62420156 | | | | | | | |
| Iceland discovery group | rs9874646 | | | 0.000330 | 1.18 | 1642 | 0.746 | 6867 | 0.713 | T |
| Iceland replication group | rs9874646 | | | 0.173800 | 1.10 | 555 | 0.721 | 3393 | 0.700 | T |
| Iceland All | rs9874646 | | | 0.000078 | 1.17 | 2197 | 0.739 | 10260 | 0.709 | T |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | | | | 0.173753 | 1.10 | 539 | | | | T |
| All groups | | | | 0.000078 | 1.17 | 2012 | | | | T |
| SG03S671 | | C03 | 191533619 | | | | | | | |
| Iceland discovery group | rs9864293 | | | 0.007956 | 1.12 | 1624 | 0.418 | 6866 | 0.391 | C |
| Iceland replication group | rs9864293 | | | 0.143800 | 1.22 | 119 | 0.424 | 3392 | 0.377 | C |
| Iceland All | rs9864293 | | | 0.000591 | 1.14 | 1743 | 0.419 | 10258 | 0.386 | C |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | | | | 0.143805 | 1.22 | 115 | | | | C |
| All groups | | | | 0.000591 | 1.14 | 1596 | | | | C |
| SG04S816 | | C04 | 21568604 | | | | | | | |
| Iceland discovery group | rs4282162 | | | 0.000562 | 1.15 | 1641 | 0.565 | 6867 | 0.529 | T |
| Iceland replication group | rs4282162 | | | 0.221000 | 1.08 | 559 | 0.547 | 3393 | 0.527 | T |
| Iceland All | rs4282162 | | | 0.000244 | 1.14 | 2200 | 0.560 | 10260 | 0.528 | T |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | | | | 0.221045 | 1.08 | 543 | | | | T |
| All groups | | | | 0.000244 | 1.14 | 2014 | | | | T |
| SG04S820 | | C04 | 45948792 | | | | | | | |
| Iceland discovery group | rs7661204 | | | 0.034390 | 1.09 | 1641 | 0.576 | 6868 | 0.555 | C |
| Iceland replication group | rs7661204 | | | 0.026880 | 1.16 | 562 | 0.583 | 3393 | 0.547 | C |
| Iceland All | rs7661204 | | | 0.002633 | 1.11 | 2203 | 0.578 | 10261 | 0.552 | C |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | | | | 0.026881 | 1.16 | 546 | | | | C |
| All groups | | | | 0.002633 | 1.11 | 2017 | | | | C |
| SG05S3061 | | C05 | 118135750 | | | | | | | |
| Iceland discovery group | rs728676 | | | 0.003297 | 1.13 | 1641 | 0.470 | 6868 | 0.440 | T |
| Iceland replication group | rs728676 | | | 0.011820 | 1.18 | 561 | 0.482 | 3392 | 0.441 | T |
| Iceland All | rs728676 | | | 0.000145 | 1.14 | 2202 | 0.473 | 10260 | 0.440 | T |
| Philadelphia | | | | NA | | | | | | |
| Atlanta | rs728676 | | | 0.101514 | 1.14 | 464 | 0.498 | 1012 | 0.465 | T |
| Durham | rs728676 | | | 0.596462 | 0.97 | 1168 | 0.484 | 718 | 0.493 | T |
| All US groups | | | | 0.513855 | 1.03 | 1632 | | | | T |
| All replication groups | | | | 0.038993 | 1.09 | 2177 | | | | T |
| All groups | | | | 0.000449 | 1.11 | 3648 | | | | T |
| SG05S3058 | | C05 | 158504417 | | | | | | | |
| Iceland discovery group | rs7442701 | | | 0.000114 | 1.22 | 1625 | 0.208 | 6865 | 0.176 | T |
| Iceland replication group | rs7442701 | | | 0.913600 | 1.02 | 120 | 0.175 | 3390 | 0.172 | T |
| Iceland All | rs7442701 | | | 0.000045 | 1.22 | 1745 | 0.205 | 10255 | 0.175 | T |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | | | | 0.913822 | 1.02 | 116 | | | | T |
| All groups | | | | 0.000045 | 1.22 | 1598 | | | | T |
| SG06S1298 | | C06 | 29516393 | | | | | | | |
| Iceland discovery group | rs2074464 | | | 0.002492 | 1.13 | 1633 | 0.475 | 6862 | 0.444 | T |
| Iceland replication group | rs2074464 | | | 0.191700 | 1.09 | 545 | 0.466 | 3390 | 0.445 | T |
| Iceland All | rs2074464 | | | 0.000945 | 1.12 | 2178 | 0.473 | 10252 | 0.444 | T |
| Philadelphia | | | | NA | | | | | | |
| Atlanta | rs2074464 | | | 0.076812 | 1.18 | 365 | 0.484 | 576 | 0.442 | T |
| Durham | rs2074464 | | | 0.290169 | 1.07 | 1200 | 0.469 | 728 | 0.451 | T |
| All US groups | | | | 0.059737 | 1.11 | 1565 | | | | T |
| All replication groups | | | | 0.022395 | 1.10 | 2094 | | | | T |
| All groups | | | | 0.000145 | 1.12 | 3559 | | | | T |
| SG07S302 | | C07 | 116579323 | | | | | | | |
| Iceland discovery group | rs1029396 | | | 0.001065 | 1.28 | 1634 | 0.089 | 6867 | 0.071 | G |
| Iceland replication group | rs1029396 | | | 0.719800 | 0.95 | 548 | 0.063 | 3393 | 0.066 | G |
| Iceland All | rs1029396 | | | 0.003796 | 1.21 | 2182 | 0.082 | 10260 | 0.069 | G |
| Philadelphia | | | | NA | | | | | | |
| Atlanta | rs1029396 | | | 0.391029 | 1.15 | 363 | 0.094 | 572 | 0.082 | G |
| Durham | rs1029396 | | | 0.053907 | 1.25 | 1212 | 0.101 | 735 | 0.083 | G |
| All US groups | | | | 0.038030 | 1.22 | 1575 | | | | G |
| All replication groups | | | | 0.138284 | 1.12 | 2107 | | | | G |
| All groups | | | | 0.000370 | 1.21 | 3573 | | | | G |
| SG08S1314 | | C08 | 18899082 | | | | | | | |
| Iceland discovery group | rs334198 | | | 0.020830 | 1.19 | 1630 | 0.090 | 6865 | 0.076 | A |
| Iceland replication group | rs334198 | | | 0.059380 | 1.25 | 549 | 0.089 | 3393 | 0.073 | A |
| Iceland All | rs334198 | | | 0.002535 | 1.21 | 2179 | 0.089 | 10258 | 0.075 | A |
| Philadelphia | rs334198 | | | 0.370833 | 0.87 | 543 | 0.088 | 475 | 0.100 | A |
| Atlanta | rs334198 | | | 0.778416 | 1.05 | 367 | 0.086 | 542 | 0.082 | A |
| Durham | | | | NA | | | | | | |
| All US groups | | | | 0.627319 | 0.95 | 910 | | | | A |
| All replication groups | | | | 0.348780 | 1.08 | 1443 | | | | A |
| All groups | | | | 0.016141 | 1.14 | 2905 | | | | A |
| SG08S1315 | | C08 | 28215758 | | | | | | | |
| Iceland discovery group | rs4732849 | | | 0.000349 | 1.18 | 1641 | 0.306 | 6868 | 0.273 | T |
| Iceland replication group | rs4732849 | | | 0.077900 | 1.14 | 550 | 0.298 | 3393 | 0.272 | T |
| Iceland All | rs4732849 | | | 0.000056 | 1.17 | 2191 | 0.304 | 10261 | 0.273 | T |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | | | | 0.077897 | 1.14 | 534 | | | | T |
| All groups | | | | 0.000056 | 1.17 | 2006 | | | | T |
| SG08S1325 | | C08 | 129065286 | | | | | | | |
| Iceland discovery group | rs2302793 | | | 0.002452 | 1.14 | 1614 | 0.462 | 6846 | 0.431 | G |
| Iceland replication group | rs2302793 | | | 0.601500 | 1.09 | 87 | 0.454 | 3370 | 0.434 | G |
| Iceland All | rs2302793 | | | 0.001920 | 1.13 | 1701 | 0.461 | 10216 | 0.432 | G |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | | | | 0.601476 | 1.09 | 84 | | | | G |
| All groups | | | | 0.001920 | 1.13 | 1557 | | | | G |
| SG08S1326 | | C08 | 129067370 | | | | | | | |
| Iceland discovery group | rs3931282 | | | 0.001650 | 1.14 | 1634 | 0.461 | 6868 | 0.428 | C |
| Iceland replication group | rs3931282 | | | 0.050490 | 1.14 | 547 | 0.463 | 3393 | 0.431 | C |
| Iceland All | rs3931282 | | | 0.000216 | 1.14 | 2181 | 0.461 | 10261 | 0.429 | C |
| Philadelphia | | | | NA | | | | | | |
| Atlanta | rs3931282 | | | 0.205449 | 0.89 | 362 | 0.438 | 587 | 0.468 | C |
| Durham | rs3931282 | | | 0.208270 | 0.92 | 1196 | 0.455 | 732 | 0.476 | C |
| All US groups | | | | 0.078904 | 0.91 | 1558 | | | | C |
| All replication groups | | | | 0.904130 | 0.99 | 2089 | | | | C |
| All groups | | | | 0.030734 | 1.07 | 3555 | | | | C |
| SG10S822 | | C10 | 45239070 | | | | | | | |
| Iceland discovery group | rs2279434 | | | 0.000031 | 1.38 | 1634 | 0.088 | 6868 | 0.065 | T |
| Iceland replication group | rs2279434 | | | 0.509900 | 1.09 | 544 | 0.071 | 3392 | 0.065 | T |
| Iceland All | rs2279434 | | | 0.000053 | 1.31 | 2178 | 0.083 | 10260 | 0.065 | T |
| Philadelphia | rs2279434 | | | | 0.81 | 532 | 0.081 | 472 | 0.097 | T |
| Atlanta | rs2279434 | | | | 0.83 | 362 | 0.070 | 513 | 0.084 | T |
| Durham | | | | NA | | | | | | |
| All US groups | | | | 0.096180 | 0.82 | 894 | | | | T |
| All replication groups | | | | 0.422694 | 0.93 | 1422 | | | | T |
| All groups | | | | 0.006627 | 1.17 | 2888 | | | | T |
| SG10S314 | | C10 | 90667504 | | | | | | | |
| Iceland discovery group | rs1412444 | | | 0.000082 | 1.18 | 1644 | 0.396 | 6861 | 0.357 | A |
| Iceland replication group | rs1412444 | | | 0.312500 | 1.07 | 582 | 0.374 | 3389 | 0.358 | A |
| Iceland All | rs1412444 | | | 0.000076 | 1.15 | 2226 | 0.390 | 10250 | 0.357 | A |
| Philadelphia | | | | NA | | | | | | |
| Atlanta | rs1412444 | | | 0.314043 | 1.11 | 367 | 0.356 | 581 | 0.333 | A |
| Durham | rs1412444 | | | 0.913736 | 1.02 | 257 | 0.368 | 121 | 0.364 | A |
| All US groups | | | | 0.360084 | 1.08 | 624 | | | | A |
| All replication groups | | | | 0.173888 | 1.07 | 1189 | | | | A |
| All groups | | | | 0.000063 | 1.14 | 2662 | | | | A |
| SG12S844 | | C12 | 21092930 | | | | | | | |
| Iceland discovery group | rs11045689 | | | 0.011540 | 1.12 | 1630 | 0.321 | 6854 | 0.297 | G |
| Iceland replication group | rs11045689 | | | 0.133900 | 1.11 | 541 | 0.318 | 3386 | 0.295 | G |
| Iceland All | rs11045689 | | | 0.002878 | 1.12 | 2171 | 0.320 | 10240 | 0.296 | G |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | | | | 0.133868 | 1.11 | 526 | | | | G |
| All groups | | | | 0.002878 | 1.12 | 1988 | | | | G |
| SG14S801 | | C14 | 67308303 | | | | | | | |
| Iceland discovery group | rs8003722 | | | 0.000343 | 1.26 | 1642 | 0.127 | 6867 | 0.104 | A |
| Iceland replication group | rs8003722 | | | 0.007542 | 1.32 | 561 | 0.125 | 3393 | 0.098 | A |
| Iceland All | rs8003722 | | | 0.000006 | 1.28 | 2203 | 0.126 | 10260 | 0.102 | A |
| Philadelphia | | | | NA | | | | | | |
| Atlanta | rs8003722 | | | 0.734831 | 1.04 | 472 | 0.151 | 1029 | 0.147 | A |
| Durham | rs8003722 | | | 0.431112 | 0.93 | 1202 | 0.135 | 735 | 0.144 | A |
| All US groups | | | | 0.708199 | 0.97 | 1674 | | | | |
| All replication groups | | | | 0.225792 | 1.08 | 2219 | | | | A |
| All groups | | | | 0.000670 | 1.16 | 3691 | | | | A |
| SG14S796 | | C14 | 98131730 | | | | | | | |
| Iceland discovery group | rs7158073 | | | 0.000697 | 1.15 | 1630 | 0.461 | 6835 | 0.426 | T |
| Iceland replication group | rs7158073 | | | 0.017800 | 1.17 | 546 | 0.477 | 3384 | 0.438 | T |
| Iceland All | rs7158073 | | | 0.000055 | 1.15 | 2176 | 0.465 | 10219 | 0.430 | T |
| Philadelphia | | | | NA | | | | | | |
| Atlanta | rs7158073 | | | 0.647425 | 1.05 | 362 | 0.419 | 575 | 0.408 | T |
| Durham | rs7158073 | | | 0.059831 | 1.14 | 1174 | 0.460 | 719 | 0.429 | T |
| All US groups | | | | 0.071211 | 1.10 | 1536 | | | | T |
| All replication groups | | | | 0.003703 | 1.13 | 2066 | | | | T |
| All groups | | | | 0.000012 | 1.14 | 3528 | | | | T |
| SG14S794_SG14S795* | | C14 | 52400957 | | | | | | | |
| Iceland discovery group | rs4444235 | rs1957860 | | 0.000323 | 0.84 | 1642 | 0.290 | 6868 | 0.326 | C |
| Iceland replication group | rs4444235 | rs1957860 | | 0.015250 | 0.83 | 561 | 0.279 | 3393 | 0.317 | C |
| Iceland All | rs4444235 | rs1957860 | | 0.000020 | 0.84 | 2203 | 0.287 | 10261 | 0.323 | C |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | rs4444235 | rs1957860 | | 0.015245 | 0.83 | 545 | | | | C |
| All groups | rs4444235 | rs1957860 | | 0.000020 | 0.84 | 2017 | | | | C |
| SG15S142 | | C15 | 24926452 | | | | | | | |
| Iceland discovery group | rs2110209 | | | 0.001794 | 1.32 | 1634 | 0.063 | 6868 | 0.049 | A |
| Iceland replication group | rs2110209 | | | 0.036840 | 1.36 | 545 | 0.059 | 3393 | 0.044 | A |
| Iceland All | rs2110209 | | | 0.000106 | 1.34 | 2179 | 0.062 | 10261 | 0.047 | A |
| Philadelphia | | | | NA | | | | | | |
| Atlanta | | | | NA | | | | | | |
| Durham | | | | NA | | | | | | |
| All US groups | | | | NA | NA | NA | | | | |
| All replication groups | | | | 0.036835 | 1.36 | 529 | | | | A |
| All groups | | | | 0.000105 | 1.34 | 1995 | | | | A |
| SG17S742 | | C17 | 17934326 | | | | | | | |
| Iceland discovery group | rs9902941 | | | 0.000041 | 1.19 | 1642 | 0.388 | 6867 | 0.348 | C |
| Iceland replication group | rs9902941 | | | 0.961500 | 1.00 | 565 | 0.351 | 3393 | 0.351 | C |
| Iceland All | rs9902941 | | | 0.000278 | 1.14 | 2207 | 0.379 | 10260 | 0.349 | C |
| Philadelphia | rs9902941 | | | 0.170324 | 1.13 | 542 | 0.423 | 474 | 0.393 | C |
| Atlanta | rs9902941 | | | 0.246078 | 1.09 | 591 | 0.372 | 1210 | 0.352 | C |
| Durham | rs9902941 | | | 0.163124 | 1.10 | 1165 | 0.378 | 736 | 0.355 | C |
| All US groups | | | | 0.024495 | 1.10 | 2298 | | | | C |
| All replication groups | | | | 0.055496 | 1.07 | 2847 | | | | C |
| All groups | | | | 0.000023 | 1.13 | 4319 | | | | C |
| SG17S743 | | C17 | 17943470 | | | | | | | |
| Iceland discovery group | rs4925119 | | | 0.000093 | 1.18 | 1630 | 0.384 | 6857 | 0.345 | A |
| Iceland replication group | rs4925119 | | | 0.975100 | 1.00 | 541 | 0.349 | 3384 | 0.349 | A |
| Iceland All | rs4925119 | | | 0.000529 | 1.13 | 2171 | 0.375 | 10241 | 0.346 | A |
| Philadelphia | rs4925119 | | | 0.312163 | 1.10 | 537 | 0.419 | 470 | 0.397 | A |
| Atlanta | rs4925119 | | | 0.155471 | 1.11 | 646 | 0.372 | 1207 | 0.349 | A |
| Durham | rs4925119 | | | 0.139467 | 1.11 | 1189 | 0.373 | 729 | 0.350 | A |
| All US groups | | | | 0.022416 | 1.10 | 2372 | | | | A |
| All replication groups | | | | 0.051193 | 1.07 | 2898 | | | | A |
| All groups | | | | 0.000037 | 1.12 | 4360 | | | | A |
| SG17S744 | | C17 | 17947855 | | | | | | | |
| Iceland discovery group | rs3183702 | | | 0.000036 | 1.19 | 1633 | 0.389 | 6866 | 0.348 | T |
| Iceland replication group | rs3183702 | | | 0.970600 | 1.00 | 546 | 0.352 | 3391 | 0.351 | T |
| Iceland All | rs3183702 | | | 0.000241 | 1.14 | 2179 | 0.380 | 10257 | 0.349 | T |
| Philadelphia | rs3183702 | | | 0.132368 | 1.15 | 531 | 0.427 | 469 | 0.393 | T |
| Atlanta | rs3183702 | | | 0.018991 | 1.26 | 362 | 0.391 | 539 | 0.337 | T |
| Durham | rs3183702 | | | 0.469038 | 1.06 | 939 | 0.368 | 572 | 0.355 | T |
| All US groups | | | | 0.011987 | 1.14 | 1832 | | | | T |
| All replication groups | | | | 0.041110 | 1.09 | 2362 | | | | T |
| All groups | | | | 0.000009 | 1.14 | 3827 | | | | T |
| SG17S746 | | C17 | 18035257 | | | | | | | |
| Iceland discovery group | rs6502622 | | | 0.000128 | 1.18 | 1641 | 0.397 | 6868 | 0.359 | G |
| Iceland replication group | rs6502622 | | | 0.699700 | 1.03 | 560 | 0.366 | 3393 | 0.360 | G |
| Iceland All | rs6502622 | | | 0.000347 | 1.14 | 2201 | 0.389 | 10261 | 0.359 | G |
| Philadelphia | rs6502622 | | | 0.277357 | 1.10 | 540 | 0.433 | 475 | 0.409 | G |
| Atlanta | rs6502622 | | | 0.041642 | 1.16 | 593 | 0.401 | 1227 | 0.366 | G |
| Durham | rs6502622 | | | 0.180470 | 1.10 | 1165 | 0.399 | 734 | 0.377 | G |
| All US groups | | | | 0.009300 | 1.12 | 2298 | | | | G |
| All replication groups | | | | 0.016554 | 1.09 | 2842 | | | | G |
| All groups | | | | 0.000010 | 1.13 | 4313 | | | | G |
| SG17S750 | | C17 | 18149041 | | | | | | | |
| Iceland discovery group | rs2955355 | | | 0.000393 | 1.17 | 1634 | 0.319 | 6868 | 0.285 | C |
| Iceland replication group | rs2955355 | | | 0.393900 | 1.06 | 547 | 0.303 | 3393 | 0.290 | C |
| Iceland All | rs2955355 | | | 0.000440 | 1.14 | 2181 | 0.315 | 10261 | 0.287 | C |
| Philadelphia | rs2955355 | | | 0.630066 | 1.05 | 544 | 0.362 | 476 | 0.352 | C |
| Atlanta | rs2955355 | | | 0.002362 | 1.37 | 360 | 0.360 | 534 | 0.291 | C |
| Durham | | | | NA | | | | | | |
| All US groups | | | | 0.016778 | 1.18 | 904 | | | | C |
| All replication groups | | | | 0.020472 | 1.12 | 1435 | | | | C |
| All groups | | | | 0.000023 | 1.15 | 2901 | | | | C |
| SG17S747 | | C17 | 18204411 | | | | | | | |
| Iceland discovery group | rs854813 | | | 0.000154 | 1.17 | 1634 | 0.425 | 6865 | 0.387 | C |
| Iceland replication group | rs854813 | | | 0.964800 | 1.00 | 543 | 0.386 | 3393 | 0.387 | C |
| Iceland All | rs854813 | | | 0.000785 | 1.13 | 2177 | 0.415 | 10258 | 0.387 | C |
| Philadelphia | rs854813 | | | 0.077440 | 1.17 | 520 | 0.468 | 462 | 0.429 | C |
| Atlanta | rs854813 | | | 0.009373 | 1.29 | 364 | 0.449 | 539 | 0.388 | C |
| Durham | | | | NA | | | | | | |
| All US groups | | | | 0.002182 | 1.23 | 884 | | | | C |
| All replication groups | | | | 0.030822 | 1.11 | 1411 | | | | C |
| All groups | | | | 0.000011 | 1.15 | 2877 | | | | C |
| SG17S749 | | C17 | 18231090 | | | | | | | |
| Iceland discovery group | rs854793 | | | 0.000495 | 1.19 | 1634 | 0.231 | 6865 | 0.201 | A |
| Iceland replication group | rs854793 | | | 0.576100 | 0.95 | 491 | 0.198 | 3392 | 0.205 | A |
| Iceland All | rs854793 | | | 0.004364 | 1.13 | 2125 | 0.223 | 10257 | 0.203 | A |
| Philadelphia | rs854793 | | | 0.456629 | 1.08 | 505 | 0.260 | 454 | 0.246 | A |
| Atlanta | rs854793 | | | 0.040285 | 1.26 | 369 | 0.255 | 539 | 0.213 | A |
| Durham | | | | NA | | | | | | |
| All US groups | | | | 0.052002 | 1.16 | 874 | | | | A |
| All replication groups | | | | 0.283691 | 1.06 | 1351 | | | | A |
| All groups | | | | 0.000591 | 1.14 | 2820 | | | | A |
| SG17S748 | | C17 | 18235585 | | | | | | | |
| Iceland discovery group | rs854787 | | | 0.000005 | 1.21 | 1635 | 0.444 | 6816 | 0.398 | C |
| Iceland replication group | rs854787 | | | 0.580200 | 0.96 | 565 | 0.390 | 3379 | 0.399 | C |
| Iceland all | rs854787 | | | 0.000171 | 1.14 | 2200 | 0.430 | 10195 | 0.398 | C |
| Philadelphia | rs854787 | | | 0.241799 | 1.11 | 542 | 0.458 | 475 | 0.433 | C |
| Atlanta | rs854787 | | | 0.001746 | 1.36 | 360 | 0.475 | 507 | 0.399 | C |
| Durham | | | | NA | | | | | | |
| All US groups | | | | 0.002955 | 1.22 | 902 | | | | C |
| All replication groups | | | | 0.085873 | 1.08 | 1451 | | | | C |
| All groups | | | | 0.000002 | 1.16 | 2916 | | | | C |

*2-marker haplotype consisting of markers SG14S794 and SG14S795

### Example 2

Further genome-wide genotyping of Icelandic patients diagnosed with coronary artery disease and/or myocardial infarction was performed. Recruitment and phenotyping was described above under Example, and all methods involved were as described in the above.

Descriptions of the additional replication cohorts are as follows:

Cohort from Italy: The subjects from Verona were enrolled into the Verona Heart Project, which is an ongoing study aimed at identifying new risk factors for coronary artery disease (CAD) and myocardial infarction (MI) in a population of subjects with angiographic documentation of their coronary vessels. In brief, the CAD group had angiographically documented severe coronary atherosclerosis, the majority of them being candidates for coronary artery bypass grafting or percutaneous coronary intervention. Control subjects were selected such that they had normal coronary arteries, being submitted to coronary angiography for reasons other than CAD. Controls with history or clinical evidence of atherosclerosis in vascular districts beyond the coronary bed were excluded. Information on MI diagnoses was gathered through medical records showing diagnostic electrocardiogram and enzyme changes, and/or the typical sequelae of MI on ventricular angiography. The study was approved by local Ethical Committee. Informed consent was obtained from all the patients after a full explanation of the study.

Cohort from Johns Hopkins: The study group from Baltimore is comprised of The Johns Hopkins University Sibling and Family Heart Study, G-Cal, and GeneSTAR studies. The subjects consisted of families identified from 1983-2006 from probands with premature coronary disease event (before 60 years of age). Probands with documented CAD were identified at the time of hospitalization in any of the Baltimore area hospitals. Their apparently healthy siblings without CAD were recruited as well as adult offspring and spouses. Of probands, generally 24% had an MI as their index event, 65% had a PCI and/or CABG, 8.4% had angina with angiography documented CAD and were treated medically, 1.7% had sudden cardiac death, and 1.7% had angiographic documented coronary stenosis with an abnormal stress test and no symptoms.

Cohort from WTCCC: This study cohort has been described previously (Nature 447:661-78 (2007)). Summary statistics from a genome-wide association study performed on these samples is publicly available at http://www.wtccc.org.uk/info/summary_stats.shtml.

Reanalysis of the markers presented in Table 1 was performed using the expanded dataset of 3,047 individuals diagnosed with coronary artery disease, 2,395 diagnosed with myocardial infarction, and 28,321 population controls. The results are presented in Table 3.

The top 100 markers in the scan were explored further by replication analysis in six cohorts from Durham (N.C., US), Philadelphia (Pa., US), Atlanta (Ga., US), John Hopkins University (Maryland, US), Italy, and in a dataset from the Wellcom Trust Case Control Consortium (WTCCC; England). The replication cohorts combined comprise 6,108 cases and 19,396 controls. The results of the replication analysis is shown in Table 4A-4H, both separately for each cohort and a combined analysis for all the replication cohorts, as well as a combined for all the cohorts, including the Icelandic discovery cohort. Many of the variants discovered in the Icelandic discovery cohort show overall significant association in the combined replication groups. The effects are modest, which explains why some cohorts do not indicate significant association, while other cohorts do so.

In the combined analysis of all the groups (9,155 cases; 47,717 controls; Table 4H), marker rs11751605 stands out as the most significant, with a combined P-value of 1.16×10⁻⁸. This is a highly significant number, and in fact represents statistical significance at the genome-wide level, after applying the conservative Bonferroni correction for the number of statistical tests performed (approx. 300,000 in the combined analysis). It should be noted that the number of tests performed in the replication effort is much smaller; consequently, the correction to be applied to correct for the number of tests is smaller, or 100 (giving P-values of 0.05/100, or about 0.0005 as significant after correction). Several markers meet this threshold of significance.

TABLE 3
Association results for SNPs showing association to myoardial infarction (MI) and coronary artery
disease (CAD). The results are based on a genomic scan using 2,395 MI samples, 3,047 CAD
samples, and 28,321 controls. The columns indicate (1) chromosome, (2) position in NCBI Build
36, (3) the associating allele (4), rs name of the SNP, (5) frequency of the associating allele in
controls, (6) the number of controls, (7), the p-value for association to MI, (8) the Relative Risk
of association to MI, (9) the frequency of the associating allele in MI patients, (10) the number
of MI patients, (11) the p-value of association to CAD, (12) the Relative Risk of association to CAD,
(13) the frequency of the associating allele in CAD patients, (14) the number of CAD patients.

| | | Pos in | | | | | | | | | RR | f | N |
|---|---|---|---|---|---|---|---|---|---|---|---|---|---|
| Chr | SNP | Build 36 | Allele | f (con) | N (con) | P (MI) | RR (MI) | f (MI) | N (MI) | P (CAD) | (CAD) | (CAD) | (CAD) |
| C01 | rs10462018 | 7802214 | C | 0.821 | 28185 | 1.40E−02 | 1.11 | 0.836 | 2390 | 1.20E−01 | 1.06 | 0.830 | 3040 |
| C01 | rs2946534 | 18647370 | T | 0.571 | 28302 | 3.40E−04 | 1.13 | 0.599 | 2394 | 4.50E−04 | 1.11 | 0.596 | 3046 |
| C01 | rs3748744 | 19828833 | G | 0.898 | 28280 | 6.30E−02 | 1.11 | 0.907 | 2394 | 3.30E−02 | 1.11 | 0.908 | 3046 |
| C01 | rs7523192 | 19835634 | T | 0.813 | 28296 | 7.10E−02 | 1.08 | 0.824 | 2395 | 1.50E−01 | 1.06 | 0.821 | 3047 |
| C01 | rs7535952 | 19846151 | A | 0.722 | 28166 | 6.60E−02 | 1.07 | 0.735 | 2355 | 6.40E−02 | 1.06 | 0.734 | 3003 |
| C01 | rs2072671 | 20788288 | C | 0.313 | 28310 | 0.0011 | 1.12 | 0.338 | 2395 | 1.30E−03 | 1.11 | 0.335 | 3047 |
| C01 | rs12724604 | 20948045 | A | 0.037 | 28248 | 9.80E−03 | 1.23 | 0.045 | 2386 | 2.10E−02 | 1.19 | 0.043 | 3037 |
| C01 | rs12741305 | 21192774 | C | 0.038 | 28320 | 0.0087 | 1.23 | 0.046 | 2393 | 2.30E−02 | 1.18 | 0.044 | 3044 |
| C01 | rs10799128 | 29476586 | T | 0.200 | 28211 | 0.062 | 1.08 | 0.212 | 2378 | 1.40E−01 | 1.06 | 0.208 | 3029 |
| C01 | rs1932397 | 29835784 | G | 0.822 | 28320 | 1.80E−03 | 1.15 | 0.841 | 2395 | 2.80E−03 | 1.13 | 0.839 | 3047 |
| C01 | rs407838 | 30585414 | C | 0.846 | 28311 | 0.0039 | 1.14 | 0.863 | 2395 | 4.70E−03 | 1.13 | 0.861 | 3047 |
| C01 | rs593993 | 30648371 | C | 0.838 | 28311 | 0.00099 | 1.16 | 0.858 | 2392 | 9.30E−04 | 1.15 | 0.856 | 3044 |
| C01 | rs12029866 | 30657936 | G | 0.775 | 28318 | 0.0043 | 1.12 | 0.794 | 2395 | 4.40E−03 | 1.11 | 0.792 | 3047 |
| C01 | rs12126748 | 31307750 | A | 0.346 | 27660 | 0.0018 | 1.11 | 0.371 | 2341 | 9.90E−03 | 1.08 | 0.365 | 2979 |
| C01 | rs661221 | 40866417 | G | 0.805 | 28299 | 1.80E−02 | 1.10 | 0.820 | 2392 | 2.40E−01 | 1.05 | 0.811 | 3044 |
| C01 | rs7524868 | 40879361 | G | 0.306 | 28288 | 1.80E−02 | 1.09 | 0.324 | 2385 | 1.20E−01 | 1.05 | 0.316 | 3036 |
| C01 | rs7520394 | 40882456 | T | 0.649 | 28231 | 0.0014 | 1.12 | 0.674 | 2390 | 7.50E−02 | 1.06 | 0.662 | 3041 |
| C01 | rs2294512 | 54142894 | G | 0.688 | 28321 | 0.064 | 1.07 | 0.702 | 2395 | 7.00E−02 | 1.06 | 0.701 | 3047 |
| C01 | rs4927219 | 55524409 | G | 0.603 | 28319 | 4.50E−03 | 1.10 | 0.625 | 2395 | 3.80E−03 | 1.09 | 0.624 | 3047 |
| C01 | rs2991515 | 57656898 | T | 0.483 | 28319 | 0.0033 | 1.10 | 0.507 | 2395 | 7.70E−04 | 1.10 | 0.508 | 3047 |
| C01 | rs11208061 | 63165253 | A | 0.346 | 27897 | 0.0014 | 1.12 | 0.371 | 2375 | 6.10E−03 | 1.09 | 0.365 | 3012 |
| C01 | rs12134779 | 63168930 | T | 0.292 | 28320 | 0.0000094 | 1.17 | 0.325 | 2394 | 2.80E−04 | 1.12 | 0.316 | 3046 |
| C01 | rs6696619 | 63225377 | C | 0.673 | 28302 | 6.70E−02 | 1.07 | 0.687 | 2395 | 3.20E−01 | 1.03 | 0.680 | 3047 |
| C01 | rs1747924 | 64311549 | T | 0.810 | 28282 | 0.019 | 1.10 | 0.825 | 2395 | 0.23 | 1.05 | 0.817 | 3044 |
| C01 | rs2806535 | 64350358 | A | 0.483 | 28297 | 0.013 | 1.09 | 0.504 | 2394 | 0.0066 | 1.08 | 0.503 | 3043 |
| C01 | rs3818513 | 65646625 | C | 0.462 | 28320 | 0.049 | 1.07 | 0.477 | 2395 | 0.38 | 1.03 | 0.468 | 3047 |
| C01 | rs2025805 | 65722466 | C | 0.567 | 28315 | 0.023 | 1.08 | 0.585 | 2395 | 0.015 | 1.08 | 0.584 | 3047 |
| C01 | rs10489554 | 69921136 | T | 0.776 | 28311 | 0.0089 | 1.11 | 0.793 | 2395 | 0.038 | 1.08 | 0.788 | 3046 |
| C01 | rs10518531 | 76871826 | C | 0.845 | 28121 | 0.032 | 1.10 | 0.858 | 2359 | 0.06 | 1.08 | 0.855 | 3004 |
| C01 | rs1566250 | 76986952 | G | 0.703 | 28309 | 0.00084 | 1.13 | 0.728 | 2395 | 0.0011 | 1.11 | 0.725 | 3047 |
| C01 | rs1412414 | 78711365 | G | 0.620 | 28290 | 2.40E−03 | 1.11 | 0.644 | 2394 | 0.00059 | 1.11 | 0.645 | 3044 |
| C01 | rs1543675 | 78719467 | C | 0.380 | 28318 | 0.0013 | 1.11 | 0.405 | 2395 | 0.0036 | 1.09 | 0.401 | 3046 |
| C01 | rs1030414 | 81241030 | G | 0.902 | 28318 | 0.039 | 1.12 | 0.912 | 2395 | 0.046 | 1.11 | 0.911 | 3046 |
| C01 | rs7531507 | 81261795 | G | 0.922 | 28296 | 0.039 | 1.14 | 0.930 | 2394 | 0.021 | 1.14 | 0.931 | 3045 |
| C01 | rs12032817 | 82949953 | G | 0.375 | 28320 | 4.30E−03 | 1.10 | 0.398 | 2395 | 0.0046 | 1.09 | 0.396 | 3047 |
| C01 | rs1360903 | 86345051 | G | 0.508 | 28234 | 0.0055 | 1.09 | 0.531 | 2389 | 0.006 | 1.09 | 0.529 | 3040 |
| C01 | rs10801705 | 89272970 | A | 0.462 | 28318 | 0.0074 | 1.09 | 0.483 | 2394 | 0.018 | 1.07 | 0.479 | 3046 |
| C01 | rs4147861 | 94244536 | G | 0.922 | 28179 | 0.0019 | 1.22 | 0.935 | 2359 | 0.0012 | 1.21 | 0.934 | 3009 |
| C01 | rs10494048 | 106996582 | T | 0.381 | 28261 | 0.00089 | 1.12 | 0.407 | 2394 | 0.014 | 1.08 | 0.399 | 3044 |
| C01 | rs2335793 | 107945406 | C | 0.890 | 28309 | 0.006 | 1.16 | 0.904 | 2393 | 0.014 | 1.13 | 0.901 | 3045 |
| C01 | rs6688859 | 113577284 | T | 0.683 | 28320 | 0.19 | 1.05 | 0.693 | 2395 | 0.11 | 1.05 | 0.694 | 3047 |
| C01 | rs773586 | 113643259 | A | 0.488 | 28315 | 0.015 | 1.08 | 0.508 | 2395 | 0.0096 | 1.08 | 0.507 | 3047 |
| C01 | rs773588 | 113646347 | C | 0.282 | 28300 | 0.11 | 1.06 | 0.293 | 2394 | 0.023 | 1.08 | 0.297 | 3046 |
| C01 | rs2797409 | 114131725 | C | 0.248 | 28318 | 0.0024 | 1.12 | 0.269 | 2395 | 0.0036 | 1.10 | 0.266 | 3047 |
| C01 | rs2476601 | 114179091 | A | 0.129 | 28313 | 0.000031 | 1.21 | 0.152 | 2395 | 0.000097 | 1.18 | 0.148 | 3047 |
| C01 | rs1217407 | 114195271 | A | 0.247 | 28266 | 2.10E−03 | 1.12 | 0.269 | 2394 | 0.0031 | 1.10 | 0.266 | 3045 |
| C01 | rs1970568 | 149842380 | T | 0.815 | 28313 | 0.001 | 1.15 | 0.835 | 2395 | 0.021 | 1.09 | 0.828 | 3047 |
| C01 | rs6702842 | 149874720 | G | 0.272 | 28099 | 0.0072 | 1.10 | 0.292 | 2379 | 0.072 | 1.06 | 0.284 | 3029 |
| C01 | rs2495392 | 149943873 | G | 0.278 | 28312 | 0.0038 | 1.11 | 0.299 | 2395 | 0.052 | 1.07 | 0.291 | 3046 |
| C01 | rs12066445 | 150007044 | A | 0.302 | 28320 | 5.90E−04 | 1.13 | 0.327 | 2395 | 0.0094 | 1.09 | 0.319 | 3047 |
| C01 | rs9826 | 150045523 | G | 0.329 | 28106 | 0.0021 | 1.11 | 0.353 | 2377 | 0.03 | 1.07 | 0.344 | 3022 |
| C01 | rs949969 | 150059438 | T | 0.199 | 28317 | 0.016 | 1.10 | 0.214 | 2395 | 0.039 | 1.08 | 0.211 | 3047 |
| C01 | rs4845552 | 151746622 | G | 0.128 | 28269 | 0.0025 | 1.15 | 0.145 | 2393 | 0.00041 | 1.16 | 0.146 | 3044 |
| C01 | rs822519 | 154107330 | A | 0.935 | 28320 | 0.024 | 1.17 | 0.944 | 2395 | 0.025 | 1.15 | 0.943 | 3047 |
| C01 | rs12567958 | 154861280 | C | 0.265 | 28272 | 0.024 | 1.09 | 0.282 | 2392 | 0.075 | 1.06 | 0.277 | 3043 |
| C01 | rs4971177 | 156256292 | T | 0.914 | 28315 | 5.20E−04 | 1.24 | 0.929 | 2395 | 0.0015 | 1.19 | 0.927 | 3047 |
| C01 | rs2651860 | 163647679 | T | 0.785 | 28321 | 0.011 | 1.11 | 0.801 | 2395 | 0.012 | 1.10 | 0.800 | 3046 |
| C01 | rs11582943 | 169209225 | A | 0.941 | 28257 | 0.0019 | 1.26 | 0.953 | 2388 | 0.00098 | 1.25 | 0.952 | 3039 |
| C01 | rs12033847 | 174294601 | T | 0.460 | 27452 | 0.011 | 1.09 | 0.481 | 2360 | 0.0029 | 1.09 | 0.482 | 2999 |
| C01 | rs2175177 | 174450679 | A | 0.461 | 28011 | 6.20E−03 | 1.09 | 0.483 | 2386 | 0.0016 | 1.10 | 0.484 | 3030 |
| C01 | rs4651384 | 185685027 | G | 0.687 | 28319 | 0.0025 | 1.11 | 0.709 | 2395 | 0.028 | 1.07 | 0.702 | 3047 |
| C01 | rs2250227 | 185696690 | C | 0.337 | 28310 | 0.0058 | 1.10 | 0.358 | 2395 | 0.021 | 1.07 | 0.353 | 3047 |
| C01 | rs1339083 | 185819288 | A | 0.345 | 28315 | 0.01 | 1.09 | 0.365 | 2395 | 0.0018 | 1.10 | 0.367 | 3047 |
| C01 | rs2383658 | 185847926 | G | 0.394 | 28312 | 0.022 | 1.08 | 0.412 | 2395 | 0.0057 | 1.09 | 0.414 | 3047 |
| C01 | rs792446 | 210623301 | G | 0.617 | 28317 | 0.016 | 1.09 | 0.636 | 2395 | 0.052 | 1.06 | 0.631 | 3046 |
| C01 | rs11118175 | 217155936 | G | 0.077 | 28315 | 0.026 | 1.14 | 0.087 | 2395 | 0.0077 | 1.15 | 0.087 | 3047 |
| C01 | rs2642438 | 219036651 | G | 0.712 | 11355 | 0.0079 | 1.12 | 0.735 | 1767 | 0.083 | 1.07 | 0.726 | 2084 |
| C01 | rs1519463 | 219944762 | G | 0.508 | 28316 | 0.0096 | 1.09 | 0.529 | 2395 | 0.0035 | 1.09 | 0.530 | 3047 |
| C01 | rs1341449 | 239642925 | A | 0.548 | 28236 | 0.0079 | 1.09 | 0.570 | 2373 | 0.0083 | 1.08 | 0.568 | 3024 |
| C01 | rs3014559 | 239649291 | A | 0.550 | 28231 | 0.022 | 1.08 | 0.569 | 2393 | 0.015 | 1.08 | 0.568 | 3044 |
| C01 | rs12068067 | 245092561 | G | 0.774 | 28183 | 0.0059 | 1.12 | 0.793 | 2390 | 0.0064 | 1.10 | 0.791 | 3041 |
| C02 | rs7608117 | 11183423 | T | 0.487 | 28310 | 0.0037 | 1.10 | 0.511 | 2393 | 0.028 | 1.07 | 0.503 | 3044 |
| C02 | rs10929777 | 12079564 | C | 0.305 | 28316 | 1.60E−01 | 1.05 | 0.315 | 2395 | 0.22 | 1.04 | 0.313 | 3047 |
| C02 | rs11897395 | 16671068 | C | 0.890 | 28200 | 0.0085 | 1.15 | 0.903 | 2379 | 0.028 | 1.11 | 0.900 | 3028 |
| C02 | rs2342551 | 17178028 | T | 0.713 | 28317 | 0.0023 | 1.12 | 0.735 | 2395 | 0.012 | 1.09 | 0.729 | 3047 |
| C02 | rs12467812 | 19858951 | G | 0.330 | 28257 | 0.035 | 1.08 | 0.346 | 2395 | 0.018 | 1.08 | 0.346 | 3047 |
| C02 | rs10200481 | 24191365 | A | 0.110 | 28314 | 0.018 | 1.13 | 0.122 | 2395 | 0.079 | 1.09 | 0.118 | 3047 |
| C02 | rs6747236 | 35901725 | T | 0.717 | 28311 | 0.0021 | 1.12 | 0.739 | 2395 | 0.0098 | 1.09 | 0.734 | 3047 |
| C02 | rs10203034 | 35916379 | A | 0.681 | 28318 | 0.0032 | 1.11 | 0.703 | 2395 | 0.018 | 1.08 | 0.697 | 3047 |
| C02 | rs963731 | 39070377 | C | 0.954 | 27813 | 0.0019 | 1.30 | 0.964 | 2347 | 0.001 | 1.28 | 0.963 | 2991 |
| C02 | rs2168043 | 39185597 | C | 0.867 | 28278 | 0.0077 | 1.14 | 0.881 | 2395 | 0.0042 | 1.14 | 0.881 | 3047 |
| C02 | rs10202624 | 42368892 | A | 0.095 | 28319 | 0.028 | 1.13 | 0.106 | 2395 | 0.04 | 1.11 | 0.104 | 3047 |
| C02 | rs6724757 | 42384655 | A | 0.092 | 28315 | 0.042 | 1.12 | 0.101 | 2395 | 0.057 | 1.10 | 0.100 | 3047 |
| C02 | rs1991739 | 43797029 | A | 0.618 | 28313 | 0.035 | 1.07 | 0.635 | 2395 | 0.081 | 1.06 | 0.631 | 3047 |
| C02 | rs6709904 | 43933828 | A | 0.909 | 28315 | 0.058 | 1.12 | 0.918 | 2395 | 0.014 | 1.14 | 0.919 | 3046 |
| C02 | rs908571 | 45171969 | C | 0.289 | 28285 | 0.00081 | 1.13 | 0.314 | 2392 | 0.0028 | 1.10 | 0.310 | 3043 |
| C02 | rs3788982 | 49098912 | G | 0.871 | 28320 | 0.0034 | 1.16 | 0.887 | 2395 | 0.0029 | 1.15 | 0.885 | 3047 |
| C02 | rs6743414 | 49277575 | G | 0.847 | 28314 | 0.0011 | 1.16 | 0.866 | 2395 | 0.0011 | 1.15 | 0.864 | 3047 |
| C02 | rs4853116 | 75264786 | A | 0.632 | 28051 | 0.066 | 1.06 | 0.646 | 2390 | 0.015 | 1.08 | 0.649 | 3041 |
| C02 | rs317285 | 76133652 | A | 0.499 | 28318 | 0.0026 | 1.10 | 0.524 | 2395 | 0.006 | 1.09 | 0.520 | 3047 |
| C02 | rs404892 | 76157421 | C | 0.500 | 28297 | 0.002 | 1.11 | 0.525 | 2395 | 0.0052 | 1.09 | 0.521 | 3046 |
| C02 | rs443713 | 76163892 | G | 0.392 | 28316 | 0.0011 | 1.11 | 0.418 | 2395 | 0.0011 | 1.10 | 0.415 | 3047 |
| C02 | rs7563058 | 76196590 | C | 0.499 | 28313 | 0.0018 | 1.11 | 0.524 | 2395 | 0.0045 | 1.09 | 0.520 | 3047 |
| C02 | rs1595968 | 76225824 | T | 0.393 | 11401 | 0.00037 | 1.15 | 0.427 | 1770 | 0.0009 | 1.13 | 0.423 | 2089 |
| C02 | rs2861078 | 77551503 | T | 0.579 | 28249 | 0.28 | 1.04 | 0.588 | 2392 | 0.18 | 1.04 | 0.589 | 3042 |
| C02 | rs6547152 | 77634997 | C | 0.790 | 28305 | 0.12 | 1.07 | 0.801 | 2393 | 0.1 | 1.06 | 0.800 | 3044 |
| C02 | rs2861264 | 78220585 | T | 0.107 | 28303 | 0.0057 | 1.15 | 0.121 | 2394 | 0.024 | 1.11 | 0.117 | 3046 |
| C02 | rs11675205 | 85323792 | A | 0.255 | 28302 | 0.0005 | 1.14 | 0.280 | 2394 | 0.0011 | 1.11 | 0.276 | 3046 |
| C02 | rs2245113 | 118108945 | A | 0.179 | 28279 | 0.017 | 1.10 | 0.194 | 2392 | 0.08 | 1.07 | 0.189 | 3043 |
| C02 | rs1477451 | 118164844 | T | 0.126 | 28320 | 0.0012 | 1.17 | 0.144 | 2395 | 0.0099 | 1.12 | 0.139 | 3047 |
| C02 | rs1395930 | 123468570 | T | 0.499 | 28303 | 3.20E−03 | 1.10 | 0.523 | 2393 | 0.00012 | 1.12 | 0.528 | 3045 |
| C02 | rs2661006 | 123482987 | T | 0.497 | 28301 | 0.0035 | 1.10 | 0.521 | 2391 | 0.00013 | 1.12 | 0.525 | 3042 |
| C02 | rs3943703 | 127613790 | C | 0.214 | 28280 | 0.02 | 1.09 | 0.229 | 2395 | 0.06 | 1.07 | 0.225 | 3046 |
| C02 | rs4662743 | 128067278 | T | 0.515 | 28231 | 0.042 | 1.07 | 0.531 | 2392 | 0.016 | 1.07 | 0.533 | 3042 |
| C02 | rs2244871 | 128115310 | C | 0.413 | 28251 | 0.013 | 1.09 | 0.433 | 2394 | 0.015 | 1.08 | 0.430 | 3046 |
| C02 | rs2321299 | 134350130 | C | 0.571 | 28315 | 0.034 | 1.07 | 0.588 | 2395 | 0.21 | 1.04 | 0.580 | 3046 |
| C02 | rs2139311 | 134628584 | C | 0.601 | 28304 | 0.0044 | 1.10 | 0.624 | 2394 | 0.0022 | 1.10 | 0.623 | 3045 |
| C02 | rs12473666 | 134639501 | T | 0.140 | 28308 | 0.03 | 1.10 | 0.153 | 2394 | 0.11 | 1.07 | 0.149 | 3046 |
| C02 | rs10496721 | 134648929 | T | 0.175 | 28306 | 0.094 | 1.07 | 0.185 | 2394 | 0.39 | 1.03 | 0.179 | 3046 |
| C02 | rs1257208 | 134700540 | A | 0.140 | 28317 | 0.017 | 1.12 | 0.154 | 2395 | 0.0067 | 1.12 | 0.154 | 3047 |
| C02 | rs2268365 | 169802415 | G | 0.127 | 28319 | 0.038 | 1.10 | 0.138 | 2393 | 0.045 | 1.09 | 0.136 | 3045 |
| C02 | rs1837241 | 171017898 | A | 0.696 | 28315 | 0.00086 | 1.13 | 0.720 | 2395 | 0.0024 | 1.10 | 0.716 | 3047 |
| C02 | rs1399959 | 172140552 | G | 0.668 | 28291 | 0.0069 | 1.10 | 0.688 | 2393 | 0.026 | 1.07 | 0.683 | 3044 |
| C02 | rs951917 | 172220171 | T | 0.383 | 28319 | 0.015 | 1.08 | 0.403 | 2395 | 0.3 | 1.03 | 0.391 | 3047 |
| C02 | rs4668412 | 172328016 | A | 0.709 | 28319 | 0.0031 | 1.11 | 0.730 | 2395 | 0.018 | 1.08 | 0.724 | 3047 |
| C02 | rs6758704 | 172363343 | T | 0.709 | 28303 | 3.10E−03 | 1.11 | 0.730 | 2395 | 0.02 | 1.08 | 0.724 | 3047 |
| C02 | rs10165126 | 172528561 | C | 0.702 | 28297 | 0.00068 | 1.13 | 0.727 | 2394 | 0.003 | 1.10 | 0.721 | 3046 |
| C02 | rs6718013 | 172577431 | G | 0.536 | 28318 | 0.0027 | 1.10 | 0.560 | 2395 | 0.073 | 1.05 | 0.549 | 3047 |
| C02 | rs921083 | 176453128 | G | 0.781 | 28310 | 0.0092 | 1.11 | 0.798 | 2394 | 0.04 | 1.08 | 0.794 | 3044 |
| C02 | rs12621733 | 176499888 | G | 0.901 | 28320 | 0.05 | 1.12 | 0.911 | 2395 | 0.056 | 1.10 | 0.910 | 3047 |
| C02 | rs1946815 | 183006405 | G | 0.219 | 28310 | 0.019 | 1.09 | 0.235 | 2395 | 0.061 | 1.07 | 0.231 | 3047 |
| C02 | rs4666836 | 183055419 | T | 0.223 | 28298 | 0.075 | 1.07 | 0.236 | 2394 | 0.15 | 1.05 | 0.232 | 3046 |
| C02 | rs6434921 | 197828442 | A | 0.397 | 28313 | 0.01 | 1.09 | 0.418 | 2395 | 0.03 | 1.07 | 0.413 | 3045 |
| C02 | rs6704822 | 203370024 | G | 0.915 | 28086 | 0.021 | 1.15 | 0.926 | 2380 | 0.13 | 1.09 | 0.922 | 3030 |
| C02 | rs6435401 | 208537772 | A | 0.339 | 28305 | 0.015 | 1.09 | 0.358 | 2394 | 0.025 | 1.07 | 0.355 | 3044 |
| C02 | rs9288396 | 209243202 | T | 0.205 | 28308 | 0.0077 | 1.11 | 0.223 | 2395 | 0.0013 | 1.12 | 0.225 | 3047 |
| C02 | rs4442936 | 211847782 | G | 0.280 | 28075 | 0.00038 | 1.14 | 0.307 | 2367 | 0.00015 | 1.13 | 0.306 | 3016 |
| C02 | rs12329252 | 212093968 | T | 0.681 | 28321 | 0.0047 | 1.11 | 0.703 | 2395 | 0.0059 | 1.09 | 0.700 | 3047 |
| C02 | rs17406681 | 212111632 | T | 0.277 | 28319 | 0.0075 | 1.10 | 0.297 | 2394 | 0.022 | 1.08 | 0.292 | 3046 |
| C02 | rs10932529 | 214852965 | A | 0.250 | 28318 | 2.80E−02 | 1.09 | 0.266 | 2395 | 0.027 | 1.08 | 0.264 | 3047 |
| C02 | rs2216544 | 214891122 | A | 0.257 | 28310 | 0.031 | 1.08 | 0.273 | 2395 | 0.027 | 1.08 | 0.272 | 3047 |
| C02 | rs13417729 | 215894070 | C | 0.715 | 11415 | 0.0067 | 1.13 | 0.739 | 1775 | 0.011 | 1.11 | 0.736 | 2094 |
| C02 | rs1478581 | 218000897 | G | 0.821 | 28316 | 0.0043 | 1.13 | 0.839 | 2395 | 0.0014 | 1.13 | 0.839 | 3045 |
| C02 | rs9288639 | 229462042 | C | 0.080 | 28295 | 0.021 | 1.14 | 0.091 | 2392 | 0.059 | 1.11 | 0.088 | 3042 |
| C02 | rs12617590 | 234708220 | C | 0.201 | 28301 | 0.0073 | 1.11 | 0.219 | 2395 | 0.015 | 1.09 | 0.215 | 3047 |
| C02 | rs12052982 | 241760005 | A | 0.956 | 28309 | 0.00035 | 1.36 | 0.967 | 2393 | 0.00018 | 1.34 | 0.967 | 3045 |
| C03 | rs17565216 | 2631107 | C | 0.366 | 28306 | 0.0031 | 1.10 | 0.390 | 2392 | 0.0033 | 1.09 | 0.387 | 3044 |
| C03 | rs341973 | 5243162 | A | 0.508 | 28284 | 0.021 | 1.08 | 0.527 | 2393 | 0.051 | 1.06 | 0.523 | 3044 |
| C03 | rs7638636 | 6735499 | A | 0.511 | 27822 | 0.031 | 1.07 | 0.529 | 2376 | 0.11 | 1.05 | 0.523 | 3013 |
| C03 | rs978658 | 6741178 | G | 0.615 | 28313 | 0.13 | 1.05 | 0.627 | 2394 | 0.26 | 1.04 | 0.623 | 3046 |
| C03 | rs12491369 | 6764265 | G | 0.577 | 28320 | 0.31 | 1.03 | 0.585 | 2395 | 0.63 | 1.01 | 0.581 | 3047 |
| C03 | rs779710 | 7486375 | A | 0.596 | 28311 | 0.088 | 1.06 | 0.610 | 2395 | 0.15 | 1.04 | 0.607 | 3047 |
| C03 | rs752299 | 7494813 | T | 0.685 | 11413 | 1.50E−03 | 1.15 | 0.714 | 1775 | 0.0056 | 1.12 | 0.708 | 2094 |
| C03 | rs2128163 | 14269176 | G | 0.539 | 28300 | 0.046 | 1.07 | 0.556 | 2395 | 0.025 | 1.07 | 0.556 | 3047 |
| C03 | rs6772823 | 14276852 | G | 0.280 | 27955 | 0.0033 | 1.11 | 0.302 | 2370 | 0.00061 | 1.12 | 0.303 | 3014 |
| C03 | rs1870480 | 22619924 | G | 0.388 | 28314 | 0.14 | 1.05 | 0.400 | 2395 | 0.089 | 1.05 | 0.400 | 3047 |
| C03 | rs6763490 | 25122716 | C | 0.600 | 28310 | 0.00071 | 1.12 | 0.627 | 2395 | 0.007 | 1.09 | 0.619 | 3047 |
| C03 | rs13079598 | 29295837 | A | 0.906 | 28315 | 0.0015 | 1.20 | 0.920 | 2395 | 0.0022 | 1.17 | 0.918 | 3046 |
| C03 | rs2853705 | 39306976 | T | 0.209 | 28313 | 0.0043 | 1.12 | 0.228 | 2395 | 0.0065 | 1.10 | 0.226 | 3047 |
| C03 | rs1530733 | 54364155 | A | 0.155 | 28318 | 0.029 | 1.10 | 0.168 | 2395 | 0.033 | 1.09 | 0.167 | 3047 |
| C03 | rs603052 | 61765300 | T | 0.778 | 28301 | 0.0083 | 1.11 | 0.796 | 2394 | 0.041 | 1.08 | 0.791 | 3046 |
| C03 | rs1997366 | 62414626 | C | 0.798 | 28304 | 1.80E−01 | 1.06 | 0.807 | 2394 | 0.21 | 1.05 | 0.805 | 3045 |
| C03 | rs1534725 | 62420416 | T | 0.788 | 28304 | 4.80E−02 | 1.08 | 0.801 | 2394 | 0.041 | 1.08 | 0.800 | 3046 |
| C03 | rs9874646 | 62437858 | T | 0.717 | 28314 | 0.11 | 1.06 | 0.729 | 2394 | 0.16 | 1.05 | 0.726 | 3046 |
| C03 | rs1452075 | 62456103 | T | 0.739 | 28228 | 0.2 | 1.05 | 0.748 | 2386 | 0.15 | 1.05 | 0.748 | 3033 |
| C03 | rs7653410 | 63619805 | C | 0.324 | 28036 | 0.01 | 1.09 | 0.344 | 2383 | 0.051 | 1.06 | 0.338 | 3030 |
| C03 | rs1563545 | 66782776 | G | 0.530 | 28035 | 1.50E−02 | 1.08 | 0.550 | 2369 | 0.015 | 1.07 | 0.548 | 3016 |
| C03 | rs2046867 | 72863765 | T | 0.279 | 28307 | 0.0088 | 1.10 | 0.298 | 2395 | 0.021 | 1.08 | 0.294 | 3047 |
| C03 | rs7632726 | 72895436 | T | 0.310 | 28319 | 0.013 | 1.09 | 0.329 | 2395 | 0.026 | 1.07 | 0.325 | 3047 |
| C03 | rs2208 | 72954463 | A | 0.268 | 28315 | 0.0087 | 1.10 | 0.287 | 2395 | 0.024 | 1.08 | 0.283 | 3047 |
| C03 | rs4234186 | 74348999 | A | 0.945 | 28271 | 0.0037 | 1.25 | 0.956 | 2390 | 0.13 | 1.11 | 0.950 | 3040 |
| C03 | rs2055707 | 78706269 | A | 0.418 | 28182 | 0.00039 | 1.12 | 0.447 | 2392 | 0.0016 | 1.10 | 0.441 | 3044 |
| C03 | rs13075335 | 83006051 | T | 0.904 | 28256 | 1.60E−02 | 1.15 | 0.915 | 2393 | 0.047 | 1.11 | 0.913 | 3044 |
| C03 | rs1531377 | 99825932 | T | 0.177 | 28216 | 0.013 | 1.11 | 0.192 | 2391 | 0.2 | 1.05 | 0.184 | 3041 |
| C03 | rs3796139 | 99861391 | G | 0.150 | 28071 | 0.012 | 1.12 | 0.165 | 2375 | 0.2 | 1.05 | 0.157 | 3018 |
| C03 | rs9847700 | 124635632 | G | 0.851 | 28188 | 0.00017 | 1.20 | 0.872 | 2345 | 0.00021 | 1.17 | 0.870 | 2995 |
| C03 | rs6438839 | 125536191 | A | 0.078 | 28316 | 0.0077 | 1.17 | 0.090 | 2395 | 0.0044 | 1.16 | 0.090 | 3047 |
| C03 | rs10935515 | 146038973 | C | 0.576 | 26370 | 0.00024 | 1.13 | 0.606 | 2294 | 0.00051 | 1.11 | 0.602 | 2902 |
| C03 | rs4401402 | 146073240 | T | 0.584 | 28314 | 3.20E−03 | 1.10 | 0.608 | 2394 | 0.0032 | 1.09 | 0.606 | 3046 |
| C03 | rs13061557 | 146128702 | T | 0.698 | 28295 | 0.092 | 1.06 | 0.710 | 2395 | 0.13 | 1.05 | 0.708 | 3047 |
| C03 | rs10935523 | 146138781 | A | 0.740 | 28317 | 0.036 | 1.08 | 0.754 | 2395 | 0.026 | 1.08 | 0.754 | 3047 |
| C03 | rs1430435 | 170561887 | G | 0.501 | 28314 | 0.017 | 1.08 | 0.521 | 2395 | 0.056 | 1.06 | 0.515 | 3047 |
| C03 | rs756644 | 170567521 | A | 0.429 | 28313 | 2.20E−03 | 1.11 | 0.454 | 2394 | 0.023 | 1.07 | 0.446 | 3046 |
| C03 | rs1549100 | 175400363 | T | 0.732 | 28237 | 0.078 | 1.07 | 0.745 | 2386 | 0.28 | 1.04 | 0.739 | 3038 |
| C03 | rs1421420 | 175408319 | A | 0.727 | 28307 | 0.037 | 1.08 | 0.742 | 2395 | 0.15 | 1.05 | 0.737 | 3047 |
| C03 | rs2111956 | 175432500 | G | 0.763 | 28317 | 0.043 | 1.08 | 0.777 | 2395 | 0.099 | 1.06 | 0.773 | 3046 |
| C03 | rs6803944 | 186606110 | C | 0.845 | 28320 | 0.0084 | 1.13 | 0.860 | 2395 | 0.012 | 1.11 | 0.858 | 3047 |
| C03 | rs9864293 | 191695400 | C | 0.387 | 28308 | 0.00012 | 1.14 | 0.418 | 2395 | 0.00013 | 1.12 | 0.415 | 3046 |
| C03 | rs883279 | 194931805 | C | 0.907 | 28311 | 0.061 | 1.11 | 0.916 | 2394 | 0.03 | 1.12 | 0.916 | 3046 |
| C03 | rs4497978 | 195990481 | A | 0.666 | 28228 | 0.00031 | 1.14 | 0.694 | 2375 | 0.0019 | 1.10 | 0.688 | 3023 |
| C04 | rs6822427 | 3749870 | A | 0.488 | 28256 | 0.022 | 1.08 | 0.506 | 2391 | 0.016 | 1.07 | 0.506 | 3040 |
| C04 | rs3756193 | 8284241 | G | 0.461 | 28262 | 0.0072 | 1.09 | 0.483 | 2393 | 0.004 | 1.09 | 0.482 | 3043 |
| C04 | rs714086 | 9922140 | G | 0.761 | 28317 | 0.013 | 1.10 | 0.778 | 2395 | 0.11 | 1.06 | 0.771 | 3047 |
| C04 | rs4697653 | 10337857 | G | 0.524 | 28161 | 0.0021 | 1.11 | 0.549 | 2392 | 0.0081 | 1.08 | 0.543 | 3040 |
| C04 | rs2170752 | 10493178 | T | 0.576 | 28317 | 0.011 | 1.09 | 0.597 | 2395 | 0.077 | 1.05 | 0.589 | 3047 |
| C04 | rs959233 | 10511257 | G | 0.446 | 28291 | 2.60E−02 | 1.08 | 0.464 | 2394 | 0.02 | 1.07 | 0.463 | 3044 |
| C04 | rs10011464 | 10515465 | T | 0.462 | 28191 | 0.031 | 1.07 | 0.480 | 2375 | 0.038 | 1.06 | 0.478 | 3025 |
| C04 | rs7442366 | 16944686 | T | 0.824 | 28321 | 0.024 | 1.10 | 0.838 | 2395 | 0.066 | 1.08 | 0.835 | 3047 |
| C04 | rs2697684 | 17045056 | G | 0.774 | 28318 | 0.033 | 1.09 | 0.788 | 2395 | 0.018 | 1.09 | 0.788 | 3047 |
| C04 | rs1827591 | 21341284 | G | 0.538 | 28319 | 0.0079 | 1.09 | 0.560 | 2395 | 0.012 | 1.08 | 0.557 | 3047 |
| C04 | rs6448072 | 21352329 | C | 0.659 | 28318 | 0.017 | 1.09 | 0.677 | 2395 | 0.009 | 1.09 | 0.677 | 3047 |
| C04 | rs10516402 | 21407661 | C | 0.505 | 28313 | 0.0023 | 1.10 | 0.530 | 2394 | 0.0024 | 1.09 | 0.527 | 3046 |
| C04 | rs4282162 | 21426819 | T | 0.530 | 28305 | 0.002 | 1.11 | 0.555 | 2395 | 0.0031 | 1.09 | 0.552 | 3047 |
| C04 | rs4377576 | 21434824 | C | 0.541 | 28316 | 0.0034 | 1.10 | 0.565 | 2395 | 0.0044 | 1.09 | 0.562 | 3047 |
| C04 | rs2060515 | 29994854 | T | 0.114 | 28189 | 0.059 | 1.10 | 0.124 | 2379 | 0.064 | 1.09 | 0.123 | 3028 |
| C04 | rs4132132 | 30610399 | G | 0.451 | 28229 | 0.12 | 1.05 | 0.464 | 2390 | 0.17 | 1.04 | 0.461 | 3040 |
| C04 | rs10517277 | 33143198 | C | 0.102 | 28290 | 0.0086 | 1.15 | 0.115 | 2394 | 0.041 | 1.10 | 0.111 | 3046 |
| C04 | rs3849018 | 37325240 | T | 0.280 | 28319 | 0.003 | 1.11 | 0.301 | 2395 | 0.0016 | 1.11 | 0.301 | 3046 |
| C04 | rs4447895 | 40092010 | C | 0.887 | 28318 | 0.015 | 1.14 | 0.899 | 2395 | 0.0087 | 1.13 | 0.899 | 3047 |
| C04 | rs6447404 | 44714926 | G | 0.620 | 11414 | 0.015 | 1.10 | 0.643 | 1775 | 0.048 | 1.08 | 0.638 | 2094 |
| C04 | rs7661204 | 45728007 | T | 0.449 | 28320 | 0.0055 | 0.91 | 0.427 | 2395 | 0.00074 | 0.91 | 0.425 | 3047 |
| C04 | rs10026644 | 82020315 | G | 0.924 | 27964 | 0.0003 | 1.27 | 0.939 | 2382 | 0.01 | 1.16 | 0.933 | 3028 |
| C04 | rs10026120 | 82037699 | T | 0.959 | 28199 | 0.006 | 1.28 | 0.968 | 2393 | 0.03 | 1.19 | 0.965 | 3040 |
| C04 | rs6830629 | 82083395 | A | 0.975 | 28273 | 0.0036 | 1.39 | 0.982 | 2393 | 0.034 | 1.24 | 0.979 | 3043 |
| C04 | rs2589506 | 87158716 | G | 0.456 | 28313 | 0.018 | 1.08 | 0.475 | 2395 | 0.0053 | 1.09 | 0.476 | 3047 |
| C04 | rs2199309 | 87318595 | C | 0.219 | 26388 | 0.022 | 1.10 | 0.235 | 2276 | 0.0032 | 1.11 | 0.238 | 2883 |
| C04 | rs10516770 | 87391122 | T | 0.056 | 28310 | 0.083 | 1.13 | 0.062 | 2395 | 0.014 | 1.16 | 0.064 | 3047 |
| C04 | rs2924936 | 89860537 | G | 0.871 | 28318 | 0.11 | 1.08 | 0.879 | 2395 | 0.019 | 1.11 | 0.882 | 3047 |
| C04 | rs13130 | 89867868 | T | 0.872 | 28227 | 0.11 | 1.08 | 0.880 | 2388 | 0.019 | 1.11 | 0.883 | 3039 |
| C04 | rs6850673 | 96366883 | C | 0.142 | 28314 | 0.014 | 1.12 | 0.156 | 2394 | 0.0082 | 1.12 | 0.156 | 3046 |
| C04 | rs10516987 | 97050762 | G | 0.798 | 28265 | 0.0073 | 1.12 | 0.815 | 2385 | 0.019 | 1.09 | 0.812 | 3035 |
| C04 | rs1982346 | 106798203 | C | 0.058 | 27647 | 0.00038 | 1.26 | 0.072 | 2352 | 0.0016 | 1.21 | 0.069 | 2991 |
| C04 | rs6820367 | 119312058 | A | 0.353 | 28319 | 0.036 | 1.07 | 0.369 | 2395 | 0.078 | 1.06 | 0.365 | 3047 |
| C04 | rs6534683 | 129601758 | T | 0.656 | 28310 | 0.076 | 1.06 | 0.670 | 2395 | 0.034 | 1.07 | 0.671 | 3047 |
| C04 | rs7690289 | 129628963 | T | 0.637 | 27978 | 0.028 | 1.08 | 0.654 | 2381 | 0.0076 | 1.09 | 0.655 | 3023 |
| C04 | rs4549394 | 129650748 | T | 0.102 | 28313 | 0.094 | 1.09 | 0.110 | 2395 | 0.14 | 1.07 | 0.108 | 3047 |
| C04 | rs4383635 | 131414021 | A | 0.491 | 28225 | 0.031 | 1.07 | 0.508 | 2394 | 0.0099 | 1.08 | 0.510 | 3045 |
| C04 | rs1027884 | 131431891 | T | 0.497 | 28311 | 0.02 | 1.08 | 0.516 | 2395 | 0.0072 | 1.08 | 0.517 | 3047 |
| C04 | rs4864193 | 134641403 | C | 0.961 | 28307 | 0.16 | 1.13 | 0.965 | 2395 | 0.15 | 1.12 | 0.965 | 3047 |
| C04 | rs10028163 | 138689635 | G | 0.207 | 28316 | 0.0012 | 1.14 | 0.228 | 2395 | 0.00075 | 1.13 | 0.227 | 3047 |
| C04 | rs7669668 | 139472904 | T | 0.123 | 28319 | 0.02 | 1.12 | 0.136 | 2395 | 0.16 | 1.06 | 0.130 | 3047 |
| C04 | rs723794 | 147095001 | T | 0.773 | 28318 | 0.011 | 1.11 | 0.791 | 2395 | 0.037 | 1.08 | 0.786 | 3047 |
| C04 | rs2241521 | 147218561 | A | 0.239 | 28318 | 0.03 | 1.09 | 0.254 | 2395 | 0.017 | 1.09 | 0.255 | 3047 |
| C04 | rs17326199 | 161219141 | G | 0.214 | 28307 | 0.0099 | 1.11 | 0.231 | 2393 | 0.0041 | 1.11 | 0.231 | 3045 |
| C04 | rs17359034 | 161797877 | T | 0.390 | 28227 | 0.011 | 1.09 | 0.410 | 2394 | 0.044 | 1.06 | 0.404 | 3046 |
| C04 | rs12503470 | 162109433 | T | 0.383 | 28267 | 0.049 | 1.07 | 0.399 | 2394 | 0.019 | 1.07 | 0.400 | 3046 |
| C04 | rs9942159 | 162382460 | T | 0.380 | 28203 | 0.005 | 1.10 | 0.402 | 2383 | 0.0029 | 1.09 | 0.401 | 3034 |
| C04 | rs958247 | 162412247 | A | 0.289 | 28271 | 0.0036 | 1.11 | 0.310 | 2393 | 0.0014 | 1.11 | 0.310 | 3043 |
| C04 | rs9991428 | 163627494 | G | 0.961 | 28297 | 0.0014 | 1.34 | 0.971 | 2394 | 0.0013 | 1.30 | 0.970 | 3046 |
| C04 | rs11723570 | 165962251 | C | 0.509 | 28320 | 0.002 | 1.11 | 0.534 | 2395 | 0.0019 | 1.10 | 0.532 | 3047 |
| C04 | rs4695894 | 175054285 | G | 0.500 | 28257 | 0.0079 | 1.09 | 0.522 | 2393 | 0.017 | 1.07 | 0.518 | 3043 |
| C04 | rs309770 | 177690442 | G | 0.891 | 28321 | 0.027 | 1.13 | 0.902 | 2395 | 0.073 | 1.09 | 0.899 | 3047 |
| C04 | rs309800 | 177704874 | C | 0.799 | 28259 | 0.019 | 1.10 | 0.814 | 2391 | 0.01 | 1.10 | 0.814 | 3043 |
| C04 | rs1983130 | 178505657 | G | 0.415 | 28301 | 0.022 | 1.08 | 0.434 | 2394 | 0.073 | 1.06 | 0.428 | 3045 |
| C04 | rs17090640 | 180904641 | G | 0.952 | 28319 | 6.70E−03 | 1.24 | 0.961 | 2395 | 0.029 | 1.17 | 0.958 | 3047 |
| C05 | rs1697952 | 667963 | C | 0.374 | 28278 | 0.035 | 1.07 | 0.391 | 2392 | 0.052 | 1.06 | 0.388 | 3043 |
| C05 | rs1697997 | 3595548 | A | 0.918 | 28300 | 0.0099 | 1.17 | 0.929 | 2395 | 0.014 | 1.15 | 0.928 | 3046 |
| C05 | rs710998 | 3644916 | T | 0.903 | 28254 | 0.0014 | 1.20 | 0.918 | 2390 | 0.0045 | 1.16 | 0.915 | 3040 |
| C05 | rs11956711 | 3978083 | C | 0.066 | 28288 | 0.0047 | 1.19 | 0.077 | 2393 | 0.021 | 1.14 | 0.074 | 3044 |
| C05 | rs634344 | 3997125 | G | 0.066 | 28294 | 0.0042 | 1.20 | 0.078 | 2392 | 0.019 | 1.14 | 0.074 | 3042 |
| C05 | rs864267 | 5893433 | A | 0.451 | 28310 | 0.0023 | 1.10 | 0.476 | 2394 | 0.0083 | 1.08 | 0.470 | 3046 |
| C05 | rs2617549 | 5904873 | A | 0.453 | 28283 | 0.0023 | 1.11 | 0.477 | 2393 | 0.0072 | 1.08 | 0.472 | 3045 |
| C05 | rs1457122 | 6326004 | T | 0.338 | 28304 | 0.03 | 1.08 | 0.354 | 2395 | 0.081 | 1.06 | 0.350 | 3047 |
| C05 | rs870347 | 6898035 | T | 0.926 | 28313 | 0.0052 | 1.20 | 0.937 | 2395 | 0.0088 | 1.17 | 0.936 | 3047 |
| C05 | rs3213627 | 9694628 | T | 0.172 | 28296 | 0.0025 | 1.14 | 0.191 | 2393 | 0.03 | 1.09 | 0.184 | 3045 |
| C05 | rs2106687 | 9700012 | G | 0.130 | 28310 | 0.0055 | 1.14 | 0.146 | 2395 | 0.074 | 1.08 | 0.139 | 3046 |
| C05 | rs16883905 | 10009338 | T | 0.112 | 28320 | 0.00056 | 1.19 | 0.130 | 2395 | 0.00012 | 1.19 | 0.130 | 3047 |
| C05 | rs30616 | 14320173 | T | 0.852 | 28316 | 0.0016 | 1.16 | 0.870 | 2395 | 0.0018 | 1.14 | 0.868 | 3047 |
| C05 | rs7715223 | 15953124 | C | 0.819 | 28263 | 0.0012 | 1.15 | 0.839 | 2384 | 0.021 | 1.09 | 0.832 | 3036 |
| C05 | rs11743379 | 23752107 | T | 0.514 | 28310 | 0.093 | 1.06 | 0.528 | 2395 | 0.096 | 1.05 | 0.527 | 3047 |
| C05 | rs1382932 | 26922341 | C | 0.683 | 28294 | 0.0083 | 1.10 | 0.703 | 2393 | 0.0093 | 1.09 | 0.701 | 3045 |
| C05 | rs6875512 | 31367532 | A | 0.743 | 28305 | 0.011 | 1.10 | 0.760 | 2395 | 0.021 | 1.08 | 0.757 | 3047 |
| C05 | rs525735 | 38917385 | C | 0.777 | 28312 | 0.064 | 1.08 | 0.789 | 2394 | 0.18 | 1.05 | 0.785 | 3046 |
| C05 | rs357291 | 38959536 | C | 0.403 | 28138 | 0.048 | 1.07 | 0.418 | 2377 | 0.18 | 1.04 | 0.412 | 3025 |
| C05 | rs9292729 | 39061343 | C | 0.345 | 28300 | 0.044 | 1.07 | 0.361 | 2395 | 0.14 | 1.05 | 0.356 | 3047 |
| C05 | rs6880882 | 39112718 | A | 0.360 | 28252 | 0.051 | 1.07 | 0.375 | 2389 | 0.13 | 1.05 | 0.370 | 3039 |
| C05 | rs444556 | 39149695 | C | 0.310 | 28223 | 0.024 | 1.08 | 0.327 | 2391 | 0.085 | 1.06 | 0.322 | 3040 |
| C05 | rs13165359 | 40271560 | G | 0.881 | 28320 | 0.031 | 1.12 | 0.892 | 2395 | 0.16 | 1.07 | 0.888 | 3047 |
| C05 | rs13175020 | 40646263 | T | 0.817 | 28309 | 0.0022 | 1.14 | 0.836 | 2393 | 0.01 | 1.11 | 0.831 | 3045 |
| C05 | rs10038238 | 50797350 | A | 0.569 | 28268 | 0.00027 | 1.13 | 0.598 | 2393 | 0.0007 | 1.11 | 0.594 | 3043 |
| C05 | rs1423611 | 50806871 | C | 0.443 | 28301 | 0.00032 | 1.12 | 0.472 | 2395 | 0.0051 | 1.09 | 0.464 | 3047 |
| C05 | rs6449708 | 50887332 | T | 0.456 | 27678 | 0.00067 | 1.12 | 0.484 | 2369 | 0.0026 | 1.09 | 0.479 | 3009 |
| C05 | rs7708362 | 50924592 | T | 0.348 | 28252 | 0.0041 | 1.10 | 0.370 | 2381 | 0.02 | 1.07 | 0.364 | 3033 |
| C05 | rs4865673 | 50964567 | G | 0.647 | 28318 | 0.0086 | 1.09 | 0.668 | 2395 | 0.0038 | 1.09 | 0.668 | 3046 |
| C05 | rs38055 | 52596401 | T | 0.387 | 28311 | 0.052 | 1.07 | 0.403 | 2395 | 0.091 | 1.05 | 0.399 | 3047 |
| C05 | rs32496 | 55677396 | C | 0.576 | 28296 | 0.0066 | 1.09 | 0.598 | 2395 | 0.029 | 1.07 | 0.592 | 3046 |
| C05 | rs30365 | 55864542 | C | 0.900 | 28303 | 0.0018 | 1.19 | 0.914 | 2395 | 0.019 | 1.13 | 0.910 | 3046 |
| C05 | rs7720499 | 56057578 | T | 0.112 | 28276 | 0.0075 | 1.14 | 0.126 | 2394 | 0.0037 | 1.14 | 0.125 | 3045 |
| C05 | rs4700485 | 56069964 | A | 0.326 | 28316 | 0.013 | 1.09 | 0.345 | 2395 | 0.039 | 1.07 | 0.340 | 3047 |
| C05 | rs12657615 | 56099890 | C | 0.116 | 28319 | 0.012 | 1.13 | 0.129 | 2395 | 0.0046 | 1.14 | 0.129 | 3047 |
| C05 | rs832540 | 56234959 | C | 0.422 | 28307 | 0.0061 | 1.09 | 0.444 | 2395 | 0.0043 | 1.09 | 0.443 | 3047 |
| C05 | rs252890 | 56262167 | A | 0.430 | 27978 | 0.015 | 1.08 | 0.450 | 2373 | 0.0087 | 1.08 | 0.450 | 3012 |
| C05 | rs1445998 | 56303962 | A | 0.406 | 28292 | 0.015 | 1.08 | 0.425 | 2393 | 0.004 | 1.09 | 0.427 | 3045 |
| C05 | rs2408654 | 56318303 | C | 0.274 | 28293 | 0.0045 | 1.11 | 0.295 | 2395 | 0.0025 | 1.10 | 0.294 | 3047 |
| C05 | rs2962014 | 57506028 | G | 0.504 | 27810 | 0.051 | 1.07 | 0.520 | 2365 | 0.069 | 1.06 | 0.518 | 3011 |
| C05 | rs2112905 | 57520064 | A | 0.405 | 28312 | 0.015 | 1.08 | 0.424 | 2395 | 0.012 | 1.08 | 0.423 | 3047 |
| C05 | rs2441109 | 66174994 | G | 0.164 | 28313 | 0.017 | 1.11 | 0.178 | 2395 | 0.042 | 1.08 | 0.175 | 3047 |
| C05 | rs39700 | 66197800 | G | 0.375 | 28315 | 0.034 | 1.07 | 0.391 | 2394 | 0.05 | 1.06 | 0.389 | 3046 |
| C05 | rs974280 | 76535005 | G | 0.358 | 28315 | 0.0013 | 1.11 | 0.383 | 2395 | 0.0011 | 1.10 | 0.381 | 3047 |
| C05 | rs4704400 | 76586766 | T | 0.498 | 28306 | 0.0057 | 1.09 | 0.520 | 2394 | 0.023 | 1.07 | 0.514 | 3046 |
| C05 | rs251421 | 76608726 | C | 0.262 | 28278 | 0.02 | 1.09 | 0.278 | 2383 | 0.063 | 1.06 | 0.274 | 3033 |
| C05 | rs3816609 | 76757694 | A | 0.359 | 28316 | 0.03 | 1.08 | 0.376 | 2395 | 0.049 | 1.06 | 0.373 | 3047 |
| C05 | rs690280 | 76794989 | T | 0.429 | 28241 | 0.027 | 1.08 | 0.447 | 2388 | 0.1 | 1.05 | 0.441 | 3040 |
| C05 | rs251470 | 76814241 | T | 0.445 | 28294 | 0.02 | 1.08 | 0.464 | 2395 | 0.081 | 1.05 | 0.458 | 3047 |
| C05 | rs6452479 | 82055874 | C | 0.936 | 28169 | 0.014 | 1.19 | 0.945 | 2384 | 0.0067 | 1.19 | 0.945 | 3032 |
| C05 | rs6556861 | 94554256 | G | 0.570 | 28317 | 0.0082 | 1.09 | 0.591 | 2395 | 0.006 | 1.09 | 0.590 | 3047 |
| C05 | rs464906 | 94633980 | C | 0.619 | 28287 | 0.049 | 1.07 | 0.635 | 2394 | 0.051 | 1.06 | 0.633 | 3045 |
| C05 | rs950195 | 97466690 | G | 0.633 | 28228 | 0.0033 | 1.11 | 0.656 | 2395 | 0.002 | 1.10 | 0.654 | 3046 |
| C05 | rs168654 | 97924372 | T | 0.641 | 28309 | 0.053 | 1.07 | 0.656 | 2394 | 0.07 | 1.06 | 0.654 | 3046 |
| C05 | rs3776859 | 102379933 | T | 0.709 | 28311 | 0.001 | 1.13 | 0.733 | 2395 | 0.0067 | 1.09 | 0.727 | 3046 |
| C05 | rs26434 | 102391301 | T | 0.705 | 28286 | 0.0012 | 1.12 | 0.729 | 2391 | 0.0098 | 1.09 | 0.722 | 3042 |
| C05 | rs9327877 | 102409540 | T | 0.194 | 28309 | 0.00071 | 1.15 | 0.216 | 2395 | 0.0026 | 1.12 | 0.212 | 3046 |
| C05 | rs34374 | 102431920 | A | 0.673 | 28279 | 2.70E−03 | 1.11 | 0.696 | 2394 | 0.016 | 1.08 | 0.690 | 3046 |
| C05 | rs34813 | 102461308 | G | 0.710 | 28316 | 0.0015 | 1.12 | 0.733 | 2395 | 0.011 | 1.09 | 0.727 | 3047 |
| C05 | rs183752 | 102535711 | T | 0.717 | 28280 | 0.0027 | 1.12 | 0.739 | 2394 | 0.021 | 1.08 | 0.733 | 3046 |
| C05 | rs26821 | 102548299 | A | 0.710 | 28196 | 0.0014 | 1.12 | 0.733 | 2389 | 0.011 | 1.09 | 0.727 | 3041 |
| C05 | rs4957775 | 108007478 | G | 0.837 | 28311 | 0.0013 | 1.16 | 0.856 | 2395 | 0.0008 | 1.15 | 0.854 | 3047 |
| C05 | rs9326981 | 115348507 | C | 0.950 | 28315 | 0.076 | 1.15 | 0.956 | 2395 | 0.16 | 1.10 | 0.954 | 3047 |
| C05 | rs17138668 | 115379140 | C | 0.948 | 28317 | 0.0041 | 1.25 | 0.958 | 2395 | 0.014 | 1.19 | 0.956 | 3047 |
| C05 | rs728676 | 118087433 | T | 0.444 | 28310 | 0.0023 | 1.10 | 0.468 | 2394 | 0.001 | 1.10 | 0.468 | 3046 |
| C05 | rs7721883 | 119467718 | T | 0.466 | 28215 | 0.0048 | 1.10 | 0.489 | 2390 | 0.04 | 1.06 | 0.482 | 3041 |
| C05 | rs9327287 | 122547512 | G | 0.263 | 28313 | 0.012 | 1.10 | 0.281 | 2395 | 0.091 | 1.06 | 0.274 | 3047 |
| C05 | rs1476097 | 133864960 | T | 0.658 | 28313 | 0.0025 | 1.11 | 0.681 | 2394 | 0.03 | 1.07 | 0.673 | 3045 |
| C05 | rs4141140 | 135980112 | G | 0.701 | 28318 | 0.0043 | 1.11 | 0.723 | 2395 | 0.0031 | 1.10 | 0.721 | 3046 |
| C05 | rs1870739 | 153183640 | C | 0.853 | 28308 | 0.015 | 1.12 | 0.866 | 2392 | 0.037 | 1.09 | 0.863 | 3044 |
| C05 | rs1461236 | 153221364 | T | 0.854 | 28176 | 0.015 | 1.12 | 0.868 | 2381 | 0.039 | 1.09 | 0.865 | 3031 |
| C05 | rs716376 | 157479655 | G | 0.189 | 28314 | 0.034 | 1.09 | 0.202 | 2395 | 0.13 | 1.06 | 0.197 | 3046 |
| C05 | rs2964513 | 157878493 | A | 0.696 | 28222 | 0.025 | 1.08 | 0.713 | 2389 | 0.35 | 1.03 | 0.702 | 3040 |
| C05 | rs7442701 | 158456100 | T | 0.181 | 28293 | 0.00051 | 1.15 | 0.203 | 2394 | 0.005 | 1.11 | 0.197 | 3046 |
| C05 | rs7447732 | 158461713 | C | 0.320 | 28280 | 0.0033 | 1.11 | 0.343 | 2393 | 0.015 | 1.08 | 0.337 | 3045 |
| C05 | rs270661 | 158492732 | A | 0.210 | 28319 | 0.011 | 1.11 | 0.227 | 2394 | 0.025 | 1.08 | 0.223 | 3046 |
| C05 | rs270654 | 158497687 | G | 0.137 | 28318 | 0.012 | 1.12 | 0.151 | 2395 | 0.021 | 1.10 | 0.149 | 3047 |
| C05 | rs254850 | 158599309 | T | 0.220 | 28316 | 0.0011 | 1.13 | 0.242 | 2395 | 0.0082 | 1.10 | 0.236 | 3047 |
| C05 | rs4921437 | 158623529 | T | 0.216 | 28259 | 0.00049 | 1.14 | 0.240 | 2386 | 0.0022 | 1.11 | 0.235 | 3037 |
| C05 | rs1433048 | 158688423 | G | 0.193 | 27899 | 0.0074 | 1.12 | 0.211 | 2344 | 0.021 | 1.09 | 0.207 | 2989 |
| C05 | rs7709212 | 158696755 | T | 0.680 | 28305 | 0.004 | 1.11 | 0.702 | 2394 | 0.0061 | 1.09 | 0.699 | 3046 |
| C05 | rs953861 | 158705160 | G | 0.161 | 28318 | 0.0027 | 1.14 | 0.180 | 2395 | 0.052 | 1.08 | 0.172 | 3047 |
| C05 | rs10045431 | 158747111 | A | 0.266 | 28238 | 0.011 | 1.10 | 0.285 | 2364 | 0.095 | 1.06 | 0.277 | 3014 |
| C05 | rs889053 | 159028256 | T | 0.159 | 28319 | 0.012 | 1.12 | 0.174 | 2395 | 0.016 | 1.10 | 0.172 | 3047 |
| C05 | rs7705176 | 167424970 | G | 0.786 | 28317 | 0.15 | 1.06 | 0.796 | 2395 | 0.37 | 1.03 | 0.791 | 3047 |
| C05 | rs7736530 | 167453404 | G | 0.802 | 28315 | 0.093 | 1.07 | 0.813 | 2395 | 0.14 | 1.06 | 0.811 | 3047 |
| C05 | rs2617972 | 167476408 | C | 0.813 | 28316 | 0.095 | 1.07 | 0.824 | 2393 | 0.29 | 1.04 | 0.819 | 3045 |
| C05 | rs1077046 | 167478603 | C | 0.554 | 28303 | 0.0099 | 1.09 | 0.575 | 2395 | 0.064 | 1.06 | 0.567 | 3047 |
| C05 | rs7724098 | 176592176 | G | 0.289 | 28310 | 0.007 | 1.10 | 0.309 | 2392 | 0.034 | 1.07 | 0.304 | 3044 |
| C05 | rs3756566 | 178509237 | G | 0.404 | 28315 | 0.0033 | 1.10 | 0.427 | 2395 | 0.008 | 1.08 | 0.423 | 3047 |
| C06 | rs2113691 | 5382731 | C | 0.485 | 28112 | 0.016 | 1.08 | 0.505 | 2339 | 0.044 | 1.06 | 0.500 | 2987 |
| C06 | rs6459613 | 10170597 | A | 0.549 | 28236 | 0.039 | 1.07 | 0.565 | 2393 | 0.044 | 1.06 | 0.563 | 3043 |
| C06 | rs9357002 | 10958298 | T | 0.637 | 27588 | 0.002 | 1.11 | 0.661 | 2346 | 0.0021 | 1.10 | 0.659 | 2986 |
| C06 | rs1028716 | 12338790 | T | 0.061 | 28313 | 0.025 | 1.16 | 0.070 | 2395 | 0.0065 | 1.17 | 0.071 | 3047 |
| C06 | rs6940891 | 17345030 | C | 0.172 | 28319 | 0.013 | 1.11 | 0.187 | 2395 | 0.012 | 1.10 | 0.186 | 3047 |
| C06 | rs6905466 | 19958854 | T | 0.332 | 28315 | 0.0027 | 1.11 | 0.355 | 2395 | 0.0058 | 1.09 | 0.351 | 3047 |
| C06 | rs2294809 | 20707867 | G | 0.800 | 28290 | 0.027 | 1.10 | 0.814 | 2394 | 0.053 | 1.08 | 0.811 | 3046 |
| C06 | rs9393558 | 24562175 | T | 0.347 | 28292 | 0.0028 | 1.11 | 0.370 | 2395 | 0.046 | 1.06 | 0.361 | 3046 |
| C06 | rs722086 | 25536933 | T | 0.860 | 27686 | 0.0019 | 1.16 | 0.877 | 2373 | 0.0099 | 1.12 | 0.873 | 3018 |
| C06 | rs9461366 | 27418512 | G | 0.614 | 28267 | 0.011 | 1.09 | 0.634 | 2392 | 0.1 | 1.05 | 0.626 | 3043 |
| C06 | rs149951 | 28141066 | T | 0.726 | 28316 | 0.006 | 1.11 | 0.745 | 2395 | 0.13 | 1.05 | 0.735 | 3047 |
| C06 | rs203877 | 28156603 | T | 0.729 | 27802 | 0.0058 | 1.11 | 0.749 | 2347 | 0.1 | 1.06 | 0.740 | 2978 |
| C06 | rs1635 | 28335583 | G | 0.940 | 28201 | 0.00018 | 1.32 | 0.954 | 2382 | 0.00032 | 1.27 | 0.952 | 3031 |
| C06 | rs2074464 | 29516507 | T | 0.446 | 28296 | 0.0015 | 1.11 | 0.471 | 2392 | 0.006 | 1.08 | 0.466 | 3044 |
| C06 | rs2107191 | 29542274 | G | 0.439 | 28311 | 0.00083 | 1.12 | 0.466 | 2394 | 0.0033 | 1.09 | 0.460 | 3046 |
| C06 | rs1345227 | 29574935 | G | 0.376 | 28036 | 0.00079 | 1.12 | 0.402 | 2385 | 0.0011 | 1.10 | 0.399 | 3032 |
| C06 | rs3094572 | 29588972 | A | 0.385 | 28320 | 0.0012 | 1.11 | 0.410 | 2395 | 0.0021 | 1.10 | 0.407 | 3047 |
| C06 | rs3094564 | 29594324 | G | 0.384 | 28318 | 0.00099 | 1.12 | 0.410 | 2395 | 0.0017 | 1.10 | 0.407 | 3047 |
| C06 | rs166327 | 30110860 | A | 0.601 | 28296 | 0.00011 | 1.14 | 0.631 | 2395 | 0.0024 | 1.10 | 0.622 | 3047 |
| C06 | rs1864750 | 36812785 | G | 0.253 | 28236 | 0.0024 | 1.12 | 0.274 | 2391 | 0.00097 | 1.12 | 0.274 | 3042 |
| C06 | rs236444 | 36831682 | T | 0.257 | 28318 | 0.0042 | 1.11 | 0.277 | 2394 | 0.0019 | 1.11 | 0.277 | 3046 |
| C06 | rs9296225 | 37622439 | C | 0.478 | 28316 | 0.078 | 1.06 | 0.492 | 2395 | 0.13 | 1.05 | 0.489 | 3047 |
| C06 | rs9380681 | 37638433 | C | 0.311 | 28318 | 0.038 | 1.08 | 0.327 | 2395 | 0.25 | 1.04 | 0.319 | 3047 |
| C06 | rs9296304 | 39854857 | A | 0.500 | 28315 | 0.022 | 1.08 | 0.519 | 2395 | 0.03 | 1.07 | 0.516 | 3047 |
| C06 | rs9471170 | 39888226 | G | 0.887 | 28320 | 0.0047 | 1.16 | 0.901 | 2395 | 0.061 | 1.09 | 0.896 | 3047 |
| C06 | rs713383 | 42348347 | G | 0.684 | 28317 | 0.00018 | 1.14 | 0.712 | 2395 | 0.0011 | 1.11 | 0.706 | 3047 |
| C06 | rs7756421 | 57141968 | A | 0.851 | 28291 | 0.011 | 1.13 | 0.865 | 2394 | 0.017 | 1.11 | 0.863 | 3046 |
| C06 | rs1565487 | 69237128 | A | 0.860 | 28266 | 0.0024 | 1.16 | 0.877 | 2395 | 0.0025 | 1.14 | 0.875 | 3045 |
| C06 | rs9454539 | 69273869 | A | 0.880 | 28293 | 0.0025 | 1.17 | 0.895 | 2394 | 0.0022 | 1.16 | 0.894 | 3046 |
| C06 | rs10806602 | 69291821 | T | 0.877 | 11393 | 0.0019 | 1.21 | 0.896 | 1771 | 0.0011 | 1.21 | 0.896 | 2090 |
| C06 | rs7753765 | 71732495 | A | 0.144 | 28312 | 0.00014 | 1.19 | 0.166 | 2395 | 0.000047 | 1.18 | 0.165 | 3047 |
| C06 | rs9360453 | 72211053 | G | 0.710 | 11391 | 0.00044 | 1.17 | 0.741 | 1772 | 0.00034 | 1.16 | 0.739 | 2090 |
| C06 | rs182106 | 72270027 | T | 0.521 | 28315 | 0.000019 | 1.15 | 0.556 | 2394 | 0.000015 | 1.14 | 0.553 | 3046 |
| C06 | rs199623 | 72270621 | A | 0.521 | 28319 | 0.000024 | 1.15 | 0.555 | 2394 | 0.000019 | 1.13 | 0.552 | 3046 |
| C06 | rs12207816 | 72336597 | A | 0.915 | 28321 | 0.0014 | 1.22 | 0.929 | 2395 | 0.0013 | 1.20 | 0.928 | 3047 |
| C06 | rs10943606 | 79718496 | G | 0.748 | 28313 | 0.00034 | 1.15 | 0.773 | 2395 | 0.00041 | 1.13 | 0.770 | 3046 |
| C06 | rs1338023 | 79785047 | T | 0.622 | 28072 | 0.00013 | 1.14 | 0.653 | 2337 | 0.00019 | 1.12 | 0.649 | 2984 |
| C06 | rs9352688 | 79832882 | A | 0.623 | 27921 | 0.000091 | 1.14 | 0.654 | 2347 | 0.000086 | 1.13 | 0.651 | 2991 |
| C06 | rs6454860 | 92261323 | G | 0.327 | 27828 | 0.00016 | 1.14 | 0.357 | 2331 | 0.00098 | 1.11 | 0.350 | 2979 |
| C06 | rs9373729 | 104320935 | G | 0.356 | 28116 | 0.00046 | 1.13 | 0.383 | 2392 | 0.00082 | 1.11 | 0.380 | 3041 |
| C06 | rs1149305 | 105852507 | T | 0.062 | 28237 | 0.092 | 1.12 | 0.068 | 2388 | 0.18 | 1.08 | 0.066 | 3038 |
| C06 | rs845851 | 106254239 | A | 0.769 | 28278 | 0.0014 | 1.14 | 0.791 | 2395 | 0.0067 | 1.10 | 0.786 | 3047 |
| C06 | rs4946680 | 106265726 | T | 0.638 | 28315 | 0.00093 | 1.12 | 0.663 | 2395 | 0.007 | 1.09 | 0.657 | 3047 |
| C06 | rs4440505 | 106293118 | C | 0.786 | 28320 | 0.0094 | 1.11 | 0.803 | 2394 | 0.047 | 1.08 | 0.797 | 3046 |
| C06 | rs4945955 | 113999876 | T | 0.096 | 28209 | 0.0016 | 1.18 | 0.112 | 2388 | 0.00014 | 1.20 | 0.113 | 3039 |
| C06 | rs1360782 | 119475713 | A | 0.351 | 27923 | 0.028 | 1.08 | 0.368 | 2381 | 0.0084 | 1.09 | 0.369 | 3029 |
| C06 | rs9388117 | 123058137 | A | 0.676 | 28260 | 0.015 | 1.09 | 0.695 | 2394 | 0.029 | 1.07 | 0.691 | 3045 |
| C06 | rs11154416 | 127903550 | G | 0.759 | 28194 | 0.00041 | 1.15 | 0.783 | 2379 | 0.0043 | 1.11 | 0.776 | 3028 |
| C06 | rs376632 | 130100763 | G | 0.891 | 28306 | 0.019 | 1.13 | 0.902 | 2394 | 0.14 | 1.07 | 0.898 | 3046 |
| C06 | rs9376173 | 136401139 | A | 0.636 | 28291 | 0.063 | 1.07 | 0.651 | 2386 | 0.15 | 1.05 | 0.646 | 3038 |
| C06 | rs478884 | 143072921 | A | 0.878 | 28308 | 0.0091 | 1.14 | 0.892 | 2395 | 0.0037 | 1.14 | 0.892 | 3046 |
| C06 | rs6570956 | 149604532 | T | 0.736 | 28313 | 0.0054 | 1.11 | 0.756 | 2395 | 0.006 | 1.10 | 0.754 | 3047 |
| C06 | rs4512236 | 156341799 | T | 0.421 | 28311 | 0.027 | 1.08 | 0.439 | 2395 | 0.11 | 1.05 | 0.433 | 3047 |
| C06 | rs16900254 | 157876267 | A | 0.408 | 11415 | 0.0076 | 1.11 | 0.434 | 1775 | 0.013 | 1.10 | 0.430 | 2094 |
| C06 | rs2758317 | 159987277 | C | 0.427 | 28017 | 0.00011 | 1.14 | 0.458 | 2380 | 0.002 | 1.10 | 0.450 | 3028 |
| C06 | rs11751605 | 160883220 | C | 0.111 | 28085 | 0.00098 | 1.18 | 0.128 | 2387 | 0.00039 | 1.17 | 0.128 | 3033 |
| C06 | rs7739802 | 162154323 | T | 0.493 | 28266 | 0.027 | 1.08 | 0.511 | 2393 | 0.011 | 1.08 | 0.512 | 3043 |
| C06 | rs3016536 | 162157939 | G | 0.519 | 28320 | 0.02 | 1.08 | 0.538 | 2395 | 0.013 | 1.08 | 0.537 | 3047 |
| C06 | rs875785 | 162250622 | C | 0.917 | 28294 | 0.0013 | 1.22 | 0.931 | 2391 | 0.00067 | 1.21 | 0.931 | 3043 |
| C06 | rs1744503 | 165554293 | T | 0.487 | 28235 | 0.053 | 1.07 | 0.503 | 2391 | 0.02 | 1.07 | 0.504 | 3040 |
| C06 | rs9348022 | 165948264 | T | 0.659 | 28165 | 0.022 | 1.08 | 0.677 | 2385 | 0.064 | 1.06 | 0.672 | 3032 |
| C07 | rs10267593 | 1903787 | G | 0.837 | 28151 | 0.0063 | 1.13 | 0.853 | 2387 | 0.00087 | 1.15 | 0.854 | 3036 |
| C07 | rs12234501 | 8738675 | T | 0.137 | 28284 | 0.012 | 1.12 | 0.151 | 2394 | 0.002 | 1.14 | 0.153 | 3046 |
| C07 | rs917038 | 8839252 | C | 0.787 | 28193 | 0.029 | 1.09 | 0.801 | 2380 | 0.045 | 1.08 | 0.799 | 3030 |
| C07 | rs1526543 | 11723301 | T | 0.831 | 28317 | 0.0026 | 1.14 | 0.849 | 2395 | 0.0031 | 1.13 | 0.847 | 3047 |
| C07 | rs6460851 | 11868337 | G | 0.290 | 28289 | 0.0013 | 1.12 | 0.314 | 2393 | 0.0013 | 1.11 | 0.312 | 3043 |
| C07 | rs10215881 | 11880480 | C | 0.500 | 28305 | 0.0087 | 1.09 | 0.521 | 2394 | 0.02 | 1.07 | 0.517 | 3045 |
| C07 | rs2355129 | 11892064 | A | 0.366 | 28317 | 0.0049 | 1.10 | 0.388 | 2394 | 0.004 | 1.09 | 0.386 | 3046 |
| C07 | rs3735344 | 13901213 | G | 0.422 | 28199 | 0.007 | 1.09 | 0.443 | 2379 | 0.007 | 1.08 | 0.441 | 3030 |
| C07 | rs706076 | 16813149 | G | 0.145 | 28302 | 0.0057 | 1.13 | 0.161 | 2394 | 0.073 | 1.08 | 0.154 | 3046 |
| C07 | rs1029577 | 17032434 | T | 0.318 | 28274 | 0.00028 | 1.13 | 0.346 | 2391 | 0.0015 | 1.10 | 0.340 | 3043 |
| C07 | rs11766489 | 17970195 | T | 0.427 | 28308 | 0.013 | 1.08 | 0.447 | 2394 | 0.015 | 1.08 | 0.445 | 3045 |
| C07 | rs4470902 | 17974835 | C | 0.423 | 28315 | 0.008 | 1.09 | 0.444 | 2395 | 0.011 | 1.08 | 0.441 | 3047 |
| C07 | rs2269752 | 18665810 | C | 0.930 | 28307 | 0.0068 | 1.20 | 0.941 | 2395 | 0.011 | 1.17 | 0.939 | 3047 |
| C07 | rs10486378 | 19952661 | G | 0.279 | 28001 | 0.048 | 1.07 | 0.294 | 2365 | 0.15 | 1.05 | 0.289 | 3011 |
| C07 | rs2110007 | 21159866 | C | 0.942 | 28252 | 0.00095 | 1.28 | 0.954 | 2390 | 0.0036 | 1.21 | 0.952 | 3038 |
| C07 | rs4722054 | 21773017 | G | 0.088 | 28234 | 0.012 | 1.15 | 0.100 | 2373 | 0.052 | 1.10 | 0.097 | 3024 |
| C07 | rs1174943 | 21973643 | G | 0.159 | 28309 | 0.0049 | 1.13 | 0.176 | 2394 | 0.025 | 1.09 | 0.171 | 3046 |
| C07 | rs17146355 | 22181251 | A | 0.050 | 28301 | 0.039 | 1.16 | 0.057 | 2392 | 0.02 | 1.16 | 0.058 | 3044 |
| C07 | rs1542608 | 23521138 | G | 0.195 | 28319 | 0.003 | 1.13 | 0.214 | 2395 | 0.013 | 1.10 | 0.210 | 3047 |
| C07 | rs740127 | 28004900 | G | 0.598 | 28316 | 0.092 | 1.06 | 0.612 | 2394 | 0.48 | 1.02 | 0.603 | 3046 |
| C07 | rs10225945 | 28216608 | A | 0.849 | 28145 | 0.026 | 1.11 | 0.862 | 2391 | 0.096 | 1.07 | 0.858 | 3042 |
| C07 | rs10486588 | 28561447 | G | 0.230 | 28252 | 0.0091 | 1.10 | 0.248 | 2388 | 0.016 | 1.09 | 0.245 | 3039 |
| C07 | rs1976489 | 28576321 | G | 0.248 | 28319 | 0.011 | 1.10 | 0.266 | 2395 | 0.027 | 1.08 | 0.263 | 3047 |
| C07 | rs1357648 | 38150571 | T | 0.322 | 28233 | 0.0023 | 1.11 | 0.346 | 2386 | 0.014 | 1.08 | 0.339 | 3035 |
| C07 | rs1525798 | 39079105 | G | 0.306 | 28287 | 0.011 | 1.09 | 0.325 | 2395 | 0.011 | 1.08 | 0.324 | 3046 |
| C07 | rs11763728 | 39112387 | T | 0.335 | 28309 | 0.016 | 1.09 | 0.353 | 2394 | 0.063 | 1.06 | 0.348 | 3046 |
| C07 | rs4478468 | 39208006 | G | 0.312 | 28300 | 0.013 | 1.09 | 0.331 | 2395 | 0.037 | 1.07 | 0.326 | 3047 |
| C07 | rs3757564 | 43169187 | C | 0.072 | 28304 | 0.06 | 1.12 | 0.080 | 2391 | 0.065 | 1.11 | 0.079 | 3042 |
| C07 | rs10243723 | 46499109 | G | 0.972 | 28311 | 0.018 | 1.29 | 0.978 | 2394 | 0.045 | 1.21 | 0.977 | 3046 |
| C07 | rs956010 | 46744540 | A | 0.883 | 28321 | 0.0085 | 1.15 | 0.896 | 2395 | 0.00084 | 1.17 | 0.898 | 3047 |
| C07 | rs6956126 | 46756987 | G | 0.888 | 28318 | 0.019 | 1.13 | 0.900 | 2395 | 0.0026 | 1.16 | 0.902 | 3047 |
| C07 | rs13240380 | 49733144 | T | 0.436 | 28313 | 0.14 | 1.05 | 0.448 | 2395 | 0.1 | 1.05 | 0.448 | 3046 |
| C07 | rs12669163 | 50249131 | C | 0.300 | 27988 | 0.0062 | 1.10 | 0.320 | 2367 | 0.028 | 1.07 | 0.315 | 3015 |
| C07 | rs17134122 | 51042598 | A | 0.967 | 28320 | 0.093 | 1.17 | 0.972 | 2395 | 0.11 | 1.15 | 0.971 | 3047 |
| C07 | rs4527771 | 67522223 | A | 0.406 | 28212 | 0.00063 | 1.12 | 0.433 | 2385 | 0.016 | 1.07 | 0.423 | 3034 |
| C07 | rs12540706 | 67550019 | G | 0.313 | 28310 | 0.000047 | 1.15 | 0.344 | 2394 | 0.0011 | 1.11 | 0.335 | 3046 |
| C07 | rs7787362 | 73030539 | C | 0.562 | 28182 | 0.0075 | 1.09 | 0.583 | 2389 | 0.012 | 1.08 | 0.580 | 3038 |
| C07 | rs2192658 | 78023891 | T | 0.078 | 28313 | 0.032 | 1.13 | 0.088 | 2394 | 0.02 | 1.13 | 0.087 | 3045 |
| C07 | rs6467882 | 81513097 | C | 0.168 | 28308 | 0.024 | 1.10 | 0.182 | 2395 | 0.03 | 1.09 | 0.180 | 3045 |
| C07 | rs2373018 | 85426196 | G | 0.371 | 28312 | 0.015 | 1.09 | 0.391 | 2395 | 0.0087 | 1.08 | 0.390 | 3045 |
| C07 | rs10276025 | 85428427 | T | 0.371 | 28319 | 0.014 | 1.09 | 0.391 | 2395 | 0.0084 | 1.08 | 0.390 | 3047 |
| C07 | rs1012995 | 85495735 | C | 0.218 | 28145 | 0.029 | 1.09 | 0.233 | 2375 | 0.024 | 1.08 | 0.232 | 3022 |
| C07 | rs2373593 | 86874878 | A | 0.864 | 28311 | 0.0018 | 1.17 | 0.881 | 2395 | 0.0026 | 1.14 | 0.879 | 3047 |
| C07 | rs31662 | 86883520 | G | 0.864 | 28315 | 0.0018 | 1.17 | 0.881 | 2395 | 0.0021 | 1.15 | 0.879 | 3047 |
| C07 | rs999885 | 99539112 | T | 0.505 | 27962 | 0.02 | 1.08 | 0.524 | 2351 | 0.033 | 1.07 | 0.521 | 2997 |
| C07 | rs7804867 | 102820993 | A | 0.208 | 28315 | 0.056 | 1.08 | 0.221 | 2395 | 0.16 | 1.05 | 0.216 | 3046 |
| C07 | rs2270019 | 102844244 | G | 0.168 | 28300 | 0.0047 | 1.13 | 0.186 | 2392 | 0.012 | 1.10 | 0.182 | 3044 |
| C07 | rs17152161 | 102928939 | C | 0.158 | 28304 | 0.002 | 1.14 | 0.177 | 2395 | 0.00069 | 1.14 | 0.177 | 3047 |
| C07 | rs10282605 | 102949606 | C | 0.188 | 28303 | 0.00052 | 1.15 | 0.210 | 2395 | 0.00081 | 1.13 | 0.208 | 3047 |
| C07 | rs1029396 | 116812056 | G | 0.071 | 28315 | 0.0018 | 1.21 | 0.084 | 2395 | 0.000061 | 1.24 | 0.086 | 3047 |
| C07 | rs2283054 | 116913637 | A | 0.070 | 28319 | 0.0022 | 1.20 | 0.083 | 2394 | 0.000086 | 1.24 | 0.086 | 3046 |
| C07 | rs2237721 | 116920956 | C | 0.070 | 28314 | 0.0022 | 1.20 | 0.083 | 2395 | 0.000088 | 1.24 | 0.085 | 3047 |
| C07 | rs10487368 | 116927331 | T | 0.194 | 28319 | 0.034 | 1.09 | 0.208 | 2395 | 0.0033 | 1.11 | 0.212 | 3047 |
| C07 | rs2237723 | 116934783 | T | 0.197 | 28304 | 0.032 | 1.09 | 0.211 | 2395 | 0.0031 | 1.11 | 0.215 | 3047 |
| C07 | rs2237724 | 116941395 | A | 0.194 | 28320 | 0.034 | 1.09 | 0.208 | 2395 | 0.0033 | 1.11 | 0.212 | 3047 |
| C07 | rs2027945 | 116975058 | A | 0.194 | 28316 | 0.032 | 1.09 | 0.208 | 2395 | 0.0031 | 1.11 | 0.212 | 3047 |
| C07 | rs213977 | 117025681 | G | 0.412 | 28316 | 0.002 | 1.11 | 0.437 | 2395 | 0.00051 | 1.11 | 0.438 | 3047 |
| C07 | rs2299445 | 117033551 | C | 0.080 | 28315 | 0.00061 | 1.21 | 0.096 | 2394 | 0.000033 | 1.24 | 0.098 | 3046 |
| C07 | rs213987 | 117048529 | T | 0.328 | 28300 | 0.0036 | 1.11 | 0.351 | 2395 | 0.0017 | 1.10 | 0.350 | 3046 |
| C07 | rs12534186 | 117079572 | T | 0.081 | 28310 | 0.00056 | 1.22 | 0.096 | 2394 | 0.00003 | 1.24 | 0.098 | 3046 |
| C07 | rs975722 | 117120150 | G | 0.392 | 28318 | 0.0044 | 1.10 | 0.414 | 2395 | 0.0009 | 1.11 | 0.415 | 3047 |
| C07 | rs324594 | 136305863 | C | 0.266 | 28310 | 0.0023 | 1.12 | 0.289 | 2395 | 0.0018 | 1.11 | 0.287 | 3047 |
| C07 | rs11761344 | 136433359 | T | 0.119 | 28295 | 0.0042 | 1.15 | 0.134 | 2393 | 0.0046 | 1.13 | 0.132 | 3045 |
| C07 | rs7791708 | 144537777 | A | 0.677 | 28231 | 0.0046 | 1.11 | 0.698 | 2387 | 0.013 | 1.08 | 0.694 | 3035 |
| C07 | rs10271435 | 147385206 | A | 0.063 | 28313 | 0.05 | 1.14 | 0.071 | 2395 | 0.0065 | 1.17 | 0.073 | 3047 |
| C07 | rs10277774 | 150219733 | C | 0.390 | 28309 | 0.11 | 1.06 | 0.402 | 2394 | 0.11 | 1.05 | 0.401 | 3046 |
| C07 | rs11762978 | 150241033 | A | 0.654 | 28261 | 0.0057 | 1.10 | 0.676 | 2391 | 0.0029 | 1.10 | 0.675 | 3043 |
| C07 | rs2373885 | 150309013 | A | 0.749 | 28303 | 0.014 | 1.10 | 0.766 | 2395 | 0.013 | 1.09 | 0.765 | 3046 |
| C07 | rs11769878 | 157343230 | C | 0.741 | 27759 | 0.0011 | 1.13 | 0.764 | 2355 | 0.0054 | 1.10 | 0.759 | 2996 |
| C08 | rs722782 | 506479 | C | 0.892 | 28261 | 0.011 | 1.15 | 0.904 | 2363 | 0.026 | 1.12 | 0.902 | 3014 |
| C08 | rs7386449 | 1315247 | A | 0.681 | 28312 | 0.0066 | 1.10 | 0.701 | 2395 | 0.0044 | 1.10 | 0.700 | 3046 |
| C08 | rs10503316 | 5498753 | C | 0.776 | 28318 | 0.022 | 1.10 | 0.792 | 2395 | 0.058 | 1.07 | 0.788 | 3047 |
| C08 | rs6991017 | 5508780 | T | 0.793 | 28273 | 0.013 | 1.11 | 0.809 | 2393 | 0.044 | 1.08 | 0.805 | 3044 |
| C08 | rs890014 | 5835084 | C | 0.762 | 28320 | 0.15 | 1.06 | 0.772 | 2395 | 0.25 | 1.04 | 0.769 | 3047 |
| C08 | rs7006687 | 18277862 | T | 0.579 | 28306 | 0.0018 | 1.11 | 0.604 | 2392 | 0.00068 | 1.11 | 0.604 | 3044 |
| C08 | rs334198 | 18933089 | C | 0.922 | 28309 | 0.037 | 0.89 | 0.913 | 2393 | 0.082 | 0.91 | 0.915 | 3045 |
| C08 | rs11775256 | 23107430 | T | 0.274 | 28317 | 0.048 | 1.07 | 0.288 | 2395 | 0.029 | 1.07 | 0.288 | 3047 |
| C08 | rs4732849 | 28249765 | T | 0.275 | 28321 | 0.0069 | 1.10 | 0.295 | 2395 | 0.13 | 1.05 | 0.285 | 3047 |
| C08 | rs904053 | 28258855 | G | 0.224 | 28313 | 0.035 | 1.08 | 0.238 | 2395 | 0.43 | 1.03 | 0.229 | 3047 |
| C08 | rs12547858 | 32606595 | T | 0.193 | 28139 | 0.0023 | 1.13 | 0.213 | 2382 | 0.0065 | 1.11 | 0.209 | 3025 |
| C08 | rs10503927 | 32662772 | T | 0.476 | 28318 | 0.0011 | 1.11 | 0.503 | 2395 | 0.00025 | 1.11 | 0.503 | 3047 |
| C08 | rs9656744 | 32836025 | T | 0.509 | 28311 | 0.0033 | 1.10 | 0.533 | 2394 | 0.01 | 1.08 | 0.528 | 3044 |
| C08 | rs16880128 | 32852935 | G | 0.441 | 27756 | 0.0014 | 1.11 | 0.467 | 2362 | 0.014 | 1.08 | 0.459 | 3002 |
| C08 | rs7017907 | 37527288 | C | 0.088 | 28266 | 0.15 | 1.08 | 0.095 | 2385 | 0.86 | 1.01 | 0.089 | 3037 |
| C08 | rs7830101 | 39527585 | T | 0.278 | 28099 | 0.071 | 1.07 | 0.291 | 2372 | 0.027 | 1.08 | 0.292 | 3021 |
| C08 | rs7819949 | 41507078 | T | 0.274 | 28315 | 0.038 | 1.08 | 0.289 | 2395 | 0.32 | 1.03 | 0.281 | 3047 |
| C08 | rs7010934 | 59139573 | C | 0.843 | 28316 | 0.0004 | 1.18 | 0.864 | 2395 | 0.01 | 1.11 | 0.857 | 3047 |
| C08 | rs4738034 | 70924464 | C | 0.703 | 28321 | 0.0094 | 1.10 | 0.723 | 2395 | 0.016 | 1.08 | 0.720 | 3047 |
| C08 | rs3829049 | 70925162 | G | 0.710 | 28294 | 0.006 | 1.11 | 0.730 | 2393 | 0.011 | 1.09 | 0.727 | 3045 |
| C08 | rs7015359 | 70957101 | G | 0.186 | 28320 | 0.0028 | 1.13 | 0.205 | 2395 | 0.0083 | 1.10 | 0.201 | 3047 |
| C08 | rs10504537 | 73402234 | A | 0.881 | 28213 | 0.035 | 1.12 | 0.891 | 2387 | 0.027 | 1.11 | 0.891 | 3035 |
| C08 | rs1837544 | 73839615 | G | 0.243 | 28305 | 0.03 | 1.09 | 0.259 | 2395 | 0.15 | 1.05 | 0.253 | 3046 |
| C08 | rs10504576 | 75429234 | A | 0.482 | 28062 | 0.0036 | 1.10 | 0.506 | 2370 | 0.0083 | 1.08 | 0.502 | 3018 |
| C08 | rs4410896 | 75433049 | A | 0.484 | 28320 | 0.0045 | 1.10 | 0.507 | 2395 | 0.011 | 1.08 | 0.503 | 3047 |
| C08 | rs6996971 | 75455047 | C | 0.481 | 28218 | 0.0056 | 1.09 | 0.504 | 2388 | 0.012 | 1.08 | 0.500 | 3037 |
| C08 | rs10283076 | 75470386 | G | 0.482 | 28290 | 0.0045 | 1.10 | 0.505 | 2392 | 0.0087 | 1.08 | 0.501 | 3043 |
| C08 | rs2369121 | 79361197 | T | 0.720 | 28319 | 0.004 | 1.11 | 0.741 | 2395 | 0.0061 | 1.10 | 0.738 | 3047 |
| C08 | rs2607102 | 92586353 | T | 0.926 | 28218 | 0.0015 | 1.23 | 0.939 | 2380 | 0.0088 | 1.17 | 0.936 | 3028 |
| C08 | rs3102526 | 96858538 | G | 0.647 | 27995 | 0.013 | 1.09 | 0.666 | 2382 | 0.015 | 1.08 | 0.664 | 3019 |
| C08 | rs3134517 | 96859237 | G | 0.647 | 28308 | 0.016 | 1.09 | 0.666 | 2395 | 0.018 | 1.08 | 0.664 | 3047 |
| C08 | rs2440213 | 97275495 | A | 0.360 | 28316 | 0.0016 | 1.11 | 0.385 | 2395 | 0.0078 | 1.09 | 0.379 | 3047 |
| C08 | rs1992087 | 97552943 | G | 0.261 | 28309 | 0.019 | 1.09 | 0.278 | 2394 | 0.028 | 1.08 | 0.276 | 3046 |
| C08 | rs1075479 | 97849402 | A | 0.792 | 28280 | 0.017 | 1.10 | 0.808 | 2390 | 0.075 | 1.07 | 0.803 | 3039 |
| C08 | rs4620270 | 125506992 | T | 0.133 | 28307 | 0.082 | 1.09 | 0.143 | 2394 | 0.041 | 1.09 | 0.143 | 3046 |
| C08 | rs2648896 | 129169615 | C | 0.149 | 28304 | 0.0004 | 1.17 | 0.170 | 2395 | 0.00052 | 1.15 | 0.167 | 3047 |
| C08 | rs2302793 | 129177879 | G | 0.434 | 28273 | 0.0018 | 1.11 | 0.459 | 2382 | 0.0075 | 1.08 | 0.454 | 3033 |
| C08 | rs3931282 | 129179963 | T | 0.568 | 28319 | 0.0016 | 0.90 | 0.543 | 2395 | 0.007 | 0.92 | 0.548 | 3047 |
| C08 | rs10505507 | 129311983 | A | 0.923 | 28320 | 0.000057 | 1.30 | 0.939 | 2395 | 0.0019 | 1.20 | 0.935 | 3047 |
| C08 | rs4451334 | 138344030 | A | 0.064 | 28282 | 0.1 | 1.11 | 0.071 | 2390 | 0.13 | 1.09 | 0.070 | 3042 |
| C08 | rs4909759 | 139293683 | T | 0.263 | 28309 | 0.051 | 1.07 | 0.277 | 2395 | 0.013 | 1.09 | 0.279 | 3047 |
| C08 | rs4909761 | 139294744 | T | 0.264 | 28318 | 0.037 | 1.08 | 0.279 | 2395 | 0.0088 | 1.09 | 0.281 | 3047 |
| C08 | rs747551 | 143209084 | T | 0.433 | 28310 | 0.0092 | 1.09 | 0.454 | 2393 | 0.034 | 1.07 | 0.449 | 3045 |
| C08 | rs7834060 | 143220109 | G | 0.431 | 28316 | 0.015 | 1.08 | 0.450 | 2395 | 0.046 | 1.06 | 0.445 | 3047 |
| C08 | rs3934930 | 143226261 | T | 0.430 | 28320 | 0.014 | 1.08 | 0.450 | 2395 | 0.043 | 1.06 | 0.445 | 3047 |
| C08 | rs11167132 | 143245585 | T | 0.216 | 28136 | 0.0076 | 1.11 | 0.234 | 2383 | 0.031 | 1.08 | 0.230 | 3028 |
| C08 | rs9644545 | 143276606 | T | 0.475 | 28309 | 0.0002 | 1.13 | 0.505 | 2394 | 0.0004 | 1.11 | 0.501 | 3046 |
| C08 | rs5297 | 143952659 | C | 0.091 | 28314 | 0.003 | 1.18 | 0.105 | 2394 | 0.0072 | 1.14 | 0.102 | 3046 |
| C08 | rs7844961 | 143999186 | T | 0.092 | 28314 | 0.00094 | 1.19 | 0.108 | 2395 | 0.0035 | 1.15 | 0.105 | 3047 |
| C08 | rs4977114 | 144250835 | C | 0.028 | 28320 | 0.014 | 1.26 | 0.035 | 2395 | 0.039 | 1.19 | 0.033 | 3047 |
| C09 | rs2890795 | 8437732 | T | 0.579 | 28315 | 0.011 | 1.09 | 0.599 | 2395 | 0.0031 | 1.09 | 0.600 | 3046 |
| C09 | rs10119236 | 8506194 | A | 0.297 | 28304 | 0.042 | 1.07 | 0.313 | 2395 | 0.017 | 1.08 | 0.314 | 3047 |
| C09 | rs10960174 | 11743695 | G | 0.614 | 28312 | 0.00026 | 1.13 | 0.643 | 2392 | 0.000093 | 1.13 | 0.642 | 3043 |
| C09 | rs1343705 | 14600866 | G | 0.475 | 28306 | 0.0019 | 1.11 | 0.500 | 2394 | 0.0021 | 1.10 | 0.498 | 3046 |
| C09 | rs4961695 | 16318137 | C | 0.801 | 28297 | 0.00029 | 1.16 | 0.824 | 2392 | 0.00012 | 1.16 | 0.823 | 3042 |
| C09 | rs1888955 | 16319537 | A | 0.740 | 28315 | 0.00025 | 1.15 | 0.766 | 2395 | 0.0003 | 1.13 | 0.763 | 3047 |
| C09 | rs9407756 | 16321304 | G | 0.674 | 28318 | 0.0021 | 1.11 | 0.697 | 2395 | 0.013 | 1.08 | 0.691 | 3047 |
| C09 | rs12683609 | 16324761 | A | 0.572 | 28317 | 0.00017 | 1.13 | 0.602 | 2394 | 0.00038 | 1.11 | 0.598 | 3046 |
| C09 | rs1329044 | 23737791 | C | 0.156 | 28317 | 0.0092 | 1.12 | 0.172 | 2395 | 0.011 | 1.11 | 0.170 | 3047 |
| C09 | rs7038525 | 23762277 | T | 0.155 | 28251 | 0.0084 | 1.12 | 0.171 | 2393 | 0.0082 | 1.11 | 0.169 | 3045 |
| C09 | rs1452335 | 28113402 | G | 0.720 | 28196 | 0.018 | 1.09 | 0.737 | 2385 | 0.022 | 1.08 | 0.735 | 3036 |
| C09 | rs1452336 | 28114180 | A | 0.500 | 28241 | 0.013 | 1.09 | 0.520 | 2389 | 0.0021 | 1.10 | 0.523 | 3040 |
| C09 | rs1452337 | 28116482 | G | 0.800 | 28150 | 0.047 | 1.09 | 0.813 | 2377 | 0.078 | 1.07 | 0.810 | 3026 |
| C09 | rs2044961 | 28154645 | G | 0.573 | 28307 | 0.008 | 1.09 | 0.594 | 2395 | 0.00083 | 1.11 | 0.597 | 3046 |
| C09 | rs10758186 | 33091838 | G | 0.385 | 28316 | 0.052 | 1.07 | 0.400 | 2395 | 0.096 | 1.05 | 0.397 | 3047 |
| C09 | rs943535 | 72064166 | A | 0.920 | 28254 | 0.01 | 1.18 | 0.931 | 2385 | 0.0061 | 1.17 | 0.931 | 3034 |
| C09 | rs10867740 | 83153344 | C | 0.237 | 28282 | 0.0067 | 1.11 | 0.256 | 2394 | 0.028 | 1.08 | 0.251 | 3046 |
| C09 | rs3211650 | 91115047 | T | 0.091 | 28320 | 0.00074 | 1.20 | 0.107 | 2394 | 0.0018 | 1.17 | 0.104 | 3046 |
| C09 | rs3211683 | 91119689 | A | 0.090 | 28319 | 0.00071 | 1.20 | 0.107 | 2395 | 0.0019 | 1.17 | 0.104 | 3047 |
| C09 | rs7854755 | 106902871 | A | 0.202 | 28257 | 0.0036 | 1.12 | 0.221 | 2389 | 0.008 | 1.10 | 0.218 | 3038 |
| C09 | rs4979085 | 113970927 | T | 0.296 | 28305 | 0.048 | 1.07 | 0.311 | 2392 | 0.048 | 1.07 | 0.309 | 3044 |
| C09 | rs2236388 | 116209121 | G | 0.901 | 28319 | 0.048 | 1.12 | 0.910 | 2395 | 0.01 | 1.14 | 0.912 | 3047 |
| C09 | rs3780680 | 125819315 | T | 0.456 | 28295 | 0.021 | 1.08 | 0.475 | 2393 | 0.037 | 1.06 | 0.471 | 3045 |
| C09 | rs7026302 | 132818296 | G | 0.909 | 28320 | 0.001 | 1.22 | 0.924 | 2395 | 0.018 | 1.13 | 0.919 | 3047 |
| C09 | rs4917341 | 136289075 | C | 0.479 | 28257 | 0.00013 | 1.13 | 0.510 | 2392 | 0.000018 | 1.14 | 0.510 | 3041 |
| C09 | rs1930788 | 136303539 | C | 0.250 | 28321 | 0.0022 | 1.12 | 0.272 | 2395 | 0.0049 | 1.10 | 0.268 | 3047 |
| C09 | rs2297538 | 138684489 | G | 0.730 | 28226 | 0.0012 | 1.13 | 0.753 | 2395 | 0.00079 | 1.12 | 0.752 | 3047 |
| C09 | rs2385891 | 138716241 | A | 0.547 | 28259 | 0.0014 | 1.11 | 0.572 | 2387 | 0.011 | 1.08 | 0.565 | 3037 |
| C10 | rs12776254 | 2144862 | G | 0.171 | 28314 | 0.0054 | 1.12 | 0.189 | 2395 | 0.0048 | 1.11 | 0.187 | 3047 |
| C10 | rs4880711 | 5217871 | C | 0.351 | 27953 | 0.042 | 1.07 | 0.367 | 2379 | 0.26 | 1.04 | 0.359 | 3015 |
| C10 | rs7912210 | 5984421 | C | 0.963 | 28273 | 0.00083 | 1.37 | 0.973 | 2374 | 0.0009 | 1.33 | 0.972 | 3023 |
| C10 | rs541045 | 8232155 | C | 0.429 | 27990 | 0.00024 | 1.13 | 0.459 | 2351 | 0.00032 | 1.11 | 0.455 | 3000 |
| C10 | rs4747760 | 8313669 | G | 0.874 | 28308 | 0.0098 | 1.14 | 0.888 | 2395 | 0.019 | 1.11 | 0.885 | 3047 |
| C10 | rs7092384 | 8340796 | C | 0.861 | 28315 | 0.0091 | 1.13 | 0.875 | 2395 | 0.011 | 1.12 | 0.873 | 3047 |
| C10 | rs1419294 | 8347160 | T | 0.710 | 28320 | 0.0036 | 1.11 | 0.731 | 2395 | 0.0045 | 1.10 | 0.728 | 3047 |
| C10 | rs10508403 | 10448081 | T | 0.091 | 28314 | 0.0017 | 1.19 | 0.106 | 2395 | 0.01 | 1.14 | 0.102 | 3047 |
| C10 | rs2138930 | 15423774 | T | 0.110 | 28304 | 0.0065 | 1.15 | 0.124 | 2395 | 0.021 | 1.11 | 0.120 | 3046 |
| C10 | rs11596590 | 15455285 | T | 0.560 | 28303 | 0.02 | 1.08 | 0.579 | 2393 | 0.072 | 1.06 | 0.573 | 3044 |
| C10 | rs12269612 | 15475877 | T | 0.401 | 28314 | 0.0078 | 1.09 | 0.423 | 2395 | 0.024 | 1.07 | 0.418 | 3047 |
| C10 | rs1925819 | 15492765 | T | 0.560 | 28319 | 0.026 | 1.08 | 0.578 | 2395 | 0.087 | 1.05 | 0.572 | 3047 |
| C10 | rs1013406 | 15501576 | C | 0.562 | 28307 | 0.024 | 1.08 | 0.580 | 2392 | 0.087 | 1.05 | 0.575 | 3044 |
| C10 | rs2184383 | 16416276 | G | 0.807 | 28313 | 0.0019 | 1.14 | 0.827 | 2395 | 0.016 | 1.10 | 0.821 | 3047 |
| C10 | rs10828393 | 23329068 | C | 0.892 | 28259 | 0.0039 | 1.17 | 0.906 | 2392 | 0.012 | 1.13 | 0.903 | 3043 |
| C10 | rs993352 | 23347212 | G | 0.885 | 28271 | 0.011 | 1.14 | 0.898 | 2384 | 0.009 | 1.13 | 0.897 | 3032 |
| C10 | rs7086207 | 26227683 | G | 0.408 | 28303 | 0.00068 | 1.12 | 0.435 | 2394 | 0.0019 | 1.10 | 0.430 | 3046 |
| C10 | rs788053 | 29363574 | T | 0.747 | 28203 | 0.0021 | 1.12 | 0.768 | 2391 | 0.0029 | 1.11 | 0.766 | 3043 |
| C10 | rs7086224 | 30857283 | A | 0.419 | 28287 | 0.028 | 1.08 | 0.437 | 2393 | 0.04 | 1.06 | 0.434 | 3045 |
| C10 | rs2804453 | 33708138 | G | 0.110 | 28256 | 0.021 | 1.12 | 0.122 | 2392 | 0.051 | 1.09 | 0.119 | 3043 |
| C10 | rs2279434 | 45275070 | T | 0.067 | 28318 | 0.0029 | 1.20 | 0.080 | 2395 | 0.014 | 1.15 | 0.076 | 3047 |
| C10 | rs3793786 | 50413094 | C | 0.247 | 28319 | 0.3 | 1.04 | 0.255 | 2395 | 0.79 | 1.01 | 0.249 | 3047 |
| C10 | rs4948410 | 61786053 | A | 0.788 | 28175 | 0.15 | 1.06 | 0.797 | 2391 | 0.4 | 1.03 | 0.793 | 3041 |
| C10 | rs990828 | 66253744 | G | 0.417 | 28283 | 0.014 | 1.08 | 0.436 | 2392 | 0.021 | 1.07 | 0.434 | 3042 |
| C10 | rs17456902 | 69554012 | G | 0.874 | 28317 | 0.0021 | 1.17 | 0.890 | 2395 | 0.00092 | 1.16 | 0.889 | 3047 |
| C10 | rs1865017 | 78364450 | C | 0.737 | 28304 | 0.0091 | 1.10 | 0.755 | 2395 | 0.047 | 1.07 | 0.749 | 3047 |
| C10 | rs697171 | 78886007 | G | 0.912 | 28288 | 0.0084 | 1.17 | 0.923 | 2391 | 0.023 | 1.13 | 0.921 | 3042 |
| C10 | rs4617529 | 85064616 | G | 0.856 | 28321 | 0.0034 | 1.15 | 0.872 | 2395 | 0.0021 | 1.14 | 0.872 | 3047 |
| C10 | rs7921473 | 85156395 | G | 0.820 | 28314 | 0.0011 | 1.15 | 0.840 | 2395 | 0.0013 | 1.13 | 0.838 | 3047 |
| C10 | rs10787854 | 85162315 | A | 0.835 | 28318 | 0.0052 | 1.13 | 0.851 | 2395 | 0.0028 | 1.13 | 0.851 | 3047 |
| C10 | rs2349157 | 85169216 | G | 0.860 | 28317 | 0.0089 | 1.13 | 0.875 | 2395 | 0.014 | 1.11 | 0.873 | 3047 |
| C10 | rs4411234 | 85211638 | C | 0.648 | 28293 | 0.074 | 1.06 | 0.662 | 2395 | 0.027 | 1.07 | 0.663 | 3047 |
| C10 | rs10886432 | 85217844 | G | 0.648 | 28271 | 0.064 | 1.07 | 0.662 | 2394 | 0.023 | 1.07 | 0.663 | 3046 |
| C10 | rs11198993 | 85244450 | A | 0.697 | 28239 | 0.022 | 1.09 | 0.714 | 2387 | 0.0037 | 1.10 | 0.717 | 3038 |
| C10 | rs11199158 | 85297471 | G | 0.618 | 28319 | 0.024 | 1.08 | 0.635 | 2395 | 0.0062 | 1.09 | 0.637 | 3047 |
| C10 | rs11199316 | 85333276 | C | 0.724 | 27673 | 0.037 | 1.08 | 0.739 | 2358 | 0.03 | 1.08 | 0.738 | 3002 |
| C10 | rs10749535 | 88094506 | T | 0.478 | 28319 | 0.026 | 1.08 | 0.496 | 2395 | 0.048 | 1.06 | 0.492 | 3047 |
| C10 | rs4933391 | 88111974 | A | 0.393 | 28298 | 0.011 | 1.09 | 0.413 | 2395 | 0.021 | 1.07 | 0.409 | 3047 |
| C10 | rs3814614 | 88118261 | T | 0.508 | 28312 | 0.013 | 1.08 | 0.528 | 2394 | 0.013 | 1.08 | 0.526 | 3046 |
| C10 | rs1412444 | 90992907 | A | 0.361 | 28293 | 0.00037 | 1.13 | 0.389 | 2394 | 0.0014 | 1.10 | 0.383 | 3045 |
| C10 | rs2243548 | 91000293 | A | 0.512 | 28294 | 0.0007 | 1.12 | 0.540 | 2392 | 0.0087 | 1.08 | 0.532 | 3043 |
| C10 | rs2243547 | 91000459 | G | 0.331 | 28033 | 0.000027 | 1.15 | 0.363 | 2386 | 0.00066 | 1.11 | 0.355 | 3033 |
| C10 | rs1028935 | 93339072 | A | 0.189 | 28316 | 0.15 | 1.06 | 0.199 | 2395 | 0.25 | 1.04 | 0.196 | 3047 |
| C10 | rs10509657 | 95166923 | C | 0.103 | 28305 | 0.024 | 1.12 | 0.115 | 2395 | 0.078 | 1.09 | 0.111 | 3047 |
| C10 | rs10509680 | 96724329 | T | 0.052 | 28256 | 0.064 | 1.14 | 0.059 | 2389 | 0.17 | 1.09 | 0.057 | 3039 |
| C10 | rs1539089 | 102211414 | C | 0.224 | 28318 | 0.027 | 1.09 | 0.239 | 2395 | 0.0086 | 1.10 | 0.240 | 3047 |
| C10 | rs11190578 | 102228943 | T | 0.224 | 28317 | 0.027 | 1.09 | 0.239 | 2395 | 0.0085 | 1.10 | 0.240 | 3047 |
| C10 | rs9420802 | 102341775 | G | 0.210 | 11411 | 0.0064 | 1.14 | 0.232 | 1773 | 0.0074 | 1.13 | 0.230 | 2092 |
| C10 | rs4630219 | 102472174 | C | 0.099 | 11332 | 0.008 | 1.18 | 0.115 | 1768 | 0.012 | 1.16 | 0.113 | 2083 |
| C10 | rs11192557 | 107442673 | T | 0.878 | 28251 | 0.017 | 1.13 | 0.890 | 2390 | 0.019 | 1.11 | 0.889 | 3040 |
| C10 | rs1337430 | 108996443 | A | 0.796 | 28310 | 0.2 | 1.05 | 0.804 | 2392 | 0.25 | 1.04 | 0.803 | 3044 |
| C10 | rs1557227 | 119643657 | C | 0.859 | 28316 | 0.0093 | 1.13 | 0.873 | 2394 | 0.0018 | 1.15 | 0.874 | 3046 |
| C10 | rs7922488 | 120006858 | G | 0.536 | 28291 | 0.022 | 1.08 | 0.554 | 2385 | 0.051 | 1.06 | 0.550 | 3037 |
| C10 | rs2935689 | 123476616 | C | 0.839 | 28314 | 0.00098 | 1.16 | 0.858 | 2395 | 0.013 | 1.11 | 0.852 | 3047 |
| C10 | rs2936875 | 123481633 | T | 0.782 | 28304 | 0.00031 | 1.16 | 0.806 | 2394 | 0.0031 | 1.11 | 0.800 | 3046 |
| C10 | rs2239586 | 124239225 | T | 0.119 | 28306 | 0.0046 | 1.15 | 0.134 | 2395 | 0.011 | 1.12 | 0.131 | 3047 |
| C10 | rs639079 | 132085590 | G | 0.192 | 27862 | 0.00062 | 1.15 | 0.214 | 2354 | 0.0009 | 1.13 | 0.211 | 2998 |
| C10 | rs11597160 | 132163778 | A | 0.907 | 28320 | 0.0034 | 1.19 | 0.920 | 2395 | 0.015 | 1.14 | 0.917 | 3047 |
| C10 | rs1484652 | 132164140 | T | 0.906 | 28319 | 0.002 | 1.20 | 0.920 | 2395 | 0.0092 | 1.15 | 0.917 | 3047 |
| C10 | rs893504 | 132859084 | C | 0.073 | 28315 | 0.021 | 1.15 | 0.082 | 2395 | 0.0064 | 1.16 | 0.083 | 3047 |
| C11 | rs7122936 | 1331032 | A | 0.576 | 28230 | 0.00078 | 1.12 | 0.603 | 2395 | 0.0013 | 1.10 | 0.600 | 3046 |
| C11 | rs7944761 | 1361414 | T | 0.532 | 28309 | 0.0053 | 1.10 | 0.555 | 2395 | 0.0027 | 1.09 | 0.554 | 3047 |
| C11 | rs4963076 | 1379752 | T | 0.481 | 28301 | 0.011 | 1.09 | 0.502 | 2395 | 0.04 | 1.06 | 0.496 | 3047 |
| C11 | rs11041390 | 7432565 | A | 0.730 | 28304 | 0.0048 | 1.11 | 0.750 | 2394 | 0.013 | 1.09 | 0.746 | 3046 |
| C11 | rs7113222 | 7437034 | G | 0.740 | 28315 | 0.0037 | 1.12 | 0.760 | 2395 | 0.015 | 1.09 | 0.755 | 3047 |
| C11 | rs12292613 | 7580592 | T | 0.890 | 28149 | 0.0001 | 1.24 | 0.910 | 2362 | 0.00018 | 1.20 | 0.907 | 3011 |
| C11 | rs10500734 | 10898494 | A | 0.771 | 28271 | 0.006 | 1.12 | 0.789 | 2385 | 0.025 | 1.08 | 0.785 | 3035 |
| C11 | rs3935878 | 12963973 | T | 0.740 | 28267 | 0.000093 | 1.16 | 0.767 | 2392 | 0.00024 | 1.13 | 0.763 | 3039 |
| C11 | rs10832223 | 14142269 | T | 0.841 | 28293 | 0.083 | 1.08 | 0.851 | 2395 | 0.046 | 1.09 | 0.851 | 3047 |
| C11 | rs7946133 | 17748398 | T | 0.914 | 28127 | 0.02 | 1.15 | 0.925 | 2332 | 0.12 | 1.09 | 0.920 | 2977 |
| C11 | rs2237921 | 17894230 | C | 0.159 | 28318 | 0.0049 | 1.13 | 0.176 | 2395 | 0.014 | 1.10 | 0.172 | 3046 |
| C11 | rs3993323 | 17910658 | A | 0.143 | 28305 | 0.004 | 1.14 | 0.160 | 2392 | 0.0089 | 1.11 | 0.157 | 3043 |
| C11 | rs920671 | 20006628 | A | 0.187 | 28318 | 0.11 | 1.07 | 0.197 | 2395 | 0.2 | 1.05 | 0.195 | 3047 |
| C11 | rs920675 | 20006846 | A | 0.363 | 28319 | 0.0039 | 1.10 | 0.386 | 2394 | 0.0041 | 1.09 | 0.383 | 3046 |
| C11 | rs10836066 | 33455044 | T | 0.303 | 28316 | 0.00026 | 1.14 | 0.330 | 2395 | 0.042 | 1.07 | 0.317 | 3046 |
| C11 | rs1532096 | 42869929 | T | 0.860 | 28317 | 0.0012 | 1.17 | 0.878 | 2395 | 0.0024 | 1.14 | 0.875 | 3047 |
| C11 | rs7939657 | 42934015 | T | 0.557 | 28307 | 0.021 | 1.08 | 0.575 | 2395 | 0.018 | 1.07 | 0.574 | 3047 |
| C11 | rs10838271 | 44341504 | C | 0.216 | 28257 | 0.019 | 1.10 | 0.232 | 2392 | 0.034 | 1.08 | 0.229 | 3042 |
| C11 | rs717117 | 56651181 | G | 0.077 | 28317 | 0.037 | 1.13 | 0.086 | 2395 | 0.048 | 1.11 | 0.085 | 3047 |
| C11 | rs1005858 | 68613492 | G | 0.471 | 28318 | 0.0019 | 1.11 | 0.496 | 2395 | 0.015 | 1.07 | 0.489 | 3047 |
| C11 | rs7111999 | 68617234 | G | 0.471 | 27937 | 0.0018 | 1.11 | 0.496 | 2354 | 0.013 | 1.08 | 0.489 | 2996 |
| C11 | rs2924536 | 68626681 | A | 0.448 | 28308 | 0.0024 | 1.10 | 0.473 | 2394 | 0.012 | 1.08 | 0.467 | 3046 |
| C11 | rs4623925 | 82740177 | A | 0.270 | 28306 | 0.0021 | 1.12 | 0.293 | 2395 | 0.013 | 1.09 | 0.287 | 3047 |
| C11 | rs2163612 | 82856844 | G | 0.479 | 28294 | 0.000001 | 1.17 | 0.519 | 2393 | 0.0000036 | 1.15 | 0.513 | 3045 |
| C11 | rs7952084 | 83059967 | A | 0.358 | 28300 | 0.0018 | 1.11 | 0.382 | 2395 | 0.0061 | 1.09 | 0.377 | 3046 |
| C11 | rs1436622 | 90698546 | A | 0.686 | 28303 | 0.00056 | 1.13 | 0.712 | 2395 | 0.00023 | 1.13 | 0.711 | 3047 |
| C11 | rs2568712 | 91046953 | T | 0.389 | 28264 | 0.00072 | 1.12 | 0.416 | 2392 | 0.014 | 1.08 | 0.407 | 3043 |
| C11 | rs808003 | 91127230 | T | 0.554 | 28317 | 0.0003 | 1.13 | 0.583 | 2395 | 0.0053 | 1.09 | 0.574 | 3047 |
| C11 | rs9971402 | 92396199 | C | 0.872 | 28196 | 0.0066 | 1.15 | 0.887 | 2388 | 0.034 | 1.10 | 0.882 | 3038 |
| C11 | rs2658801 | 92856399 | A | 0.273 | 28317 | 0.023 | 1.09 | 0.290 | 2395 | 0.004 | 1.10 | 0.292 | 3047 |
| C11 | rs1039953 | 97062996 | C | 0.680 | 28302 | 0.011 | 1.09 | 0.700 | 2395 | 0.0066 | 1.09 | 0.699 | 3047 |
| C11 | rs7120353 | 98546758 | T | 0.903 | 28289 | 0.036 | 1.13 | 0.913 | 2395 | 0.017 | 1.13 | 0.914 | 3047 |
| C11 | rs1461684 | 98601689 | T | 0.854 | 28312 | 0.072 | 1.09 | 0.864 | 2395 | 0.045 | 1.09 | 0.864 | 3047 |
| C11 | rs11225090 | 101337818 | T | 0.966 | 28172 | 0.00053 | 1.42 | 0.976 | 2340 | 0.0024 | 1.31 | 0.974 | 2989 |
| C11 | rs1276252 | 102039767 | G | 0.255 | 28318 | 0.031 | 1.08 | 0.270 | 2395 | 0.09 | 1.06 | 0.266 | 3047 |
| C11 | rs719527 | 102945919 | C | 0.236 | 28278 | 0.019 | 1.09 | 0.252 | 2395 | 0.045 | 1.07 | 0.248 | 3047 |
| C11 | rs10502020 | 103220691 | G | 0.449 | 28319 | 0.0071 | 1.09 | 0.471 | 2395 | 0.0056 | 1.09 | 0.469 | 3046 |
| C11 | rs1509729 | 108432693 | G | 0.691 | 28318 | 0.0051 | 1.11 | 0.712 | 2395 | 0.0074 | 1.09 | 0.709 | 3047 |
| C11 | rs11604632 | 110013250 | A | 0.173 | 28295 | 0.001 | 1.15 | 0.194 | 2395 | 0.000026 | 1.17 | 0.197 | 3045 |
| C11 | rs531743 | 121625352 | A | 0.955 | 28306 | 0.00062 | 1.34 | 0.966 | 2393 | 0.0032 | 1.25 | 0.964 | 3045 |
| C11 | rs4936757 | 122257924 | C | 0.567 | 28307 | 0.011 | 1.09 | 0.588 | 2395 | 0.0084 | 1.08 | 0.587 | 3047 |
| C11 | rs7933433 | 127699660 | G | 0.648 | 28312 | 0.0055 | 1.10 | 0.670 | 2394 | 0.006 | 1.09 | 0.668 | 3046 |
| C11 | rs1395504 | 132234377 | A | 0.497 | 28319 | 0.033 | 1.07 | 0.514 | 2395 | 0.031 | 1.07 | 0.513 | 3047 |
| C11 | rs1289455 | 134148357 | T | 0.150 | 28259 | 0.0013 | 1.15 | 0.169 | 2391 | 0.00055 | 1.15 | 0.169 | 3042 |
| C11 | rs1289450 | 134151851 | A | 0.198 | 28099 | 0.00044 | 1.15 | 0.222 | 2390 | 0.00015 | 1.15 | 0.221 | 3039 |
| C12 | rs797773 | 6177827 | T | 0.401 | 28283 | 0.00077 | 1.12 | 0.428 | 2393 | 0.0082 | 1.08 | 0.420 | 3044 |
| C12 | rs2160424 | 9180220 | G | 0.556 | 28256 | 0.00048 | 1.12 | 0.585 | 2384 | 0.0016 | 1.10 | 0.579 | 3035 |
| C12 | rs3213831 | 9208040 | T | 0.547 | 28320 | 0.019 | 1.08 | 0.566 | 2395 | 0.012 | 1.08 | 0.566 | 3047 |
| C12 | rs3782677 | 9208670 | C | 0.548 | 28320 | 0.017 | 1.08 | 0.567 | 2395 | 0.011 | 1.08 | 0.566 | 3047 |
| C12 | rs9805106 | 19263624 | G | 0.822 | 28153 | 0.021 | 1.11 | 0.836 | 2380 | 0.0052 | 1.12 | 0.838 | 3028 |
| C12 | rs10743315 | 19360345 | G | 0.882 | 28166 | 0.023 | 1.13 | 0.894 | 2384 | 0.017 | 1.12 | 0.893 | 3035 |
| C12 | rs2417821 | 20081535 | T | 0.754 | 28317 | 0.014 | 1.10 | 0.771 | 2395 | 0.0042 | 1.11 | 0.772 | 3047 |
| C12 | rs11045018 | 20118148 | G | 0.752 | 28298 | 0.01 | 1.10 | 0.770 | 2394 | 0.0071 | 1.10 | 0.769 | 3046 |
| C12 | rs7299937 | 20196656 | T | 0.060 | 28243 | 0.0014 | 1.23 | 0.073 | 2394 | 0.0079 | 1.17 | 0.069 | 3045 |
| C12 | rs3764006 | 20945636 | C | 0.104 | 28304 | 0.022 | 1.13 | 0.116 | 2395 | 0.022 | 1.11 | 0.115 | 3047 |
| C12 | rs1910186 | 21065184 | C | 0.111 | 28301 | 0.01 | 1.14 | 0.124 | 2395 | 0.02 | 1.11 | 0.122 | 3047 |
| C12 | rs11045689 | 21092930 | G | 0.301 | 28210 | 0.017 | 1.09 | 0.319 | 2386 | 0.015 | 1.08 | 0.317 | 3037 |
| C12 | rs2955503 | 22090132 | A | 0.655 | 28289 | 0.00054 | 1.13 | 0.681 | 2394 | 0.000033 | 1.14 | 0.684 | 3045 |
| C12 | rs2171392 | 22797592 | A | 0.196 | 28289 | 0.0068 | 1.12 | 0.213 | 2394 | 0.011 | 1.10 | 0.211 | 3045 |
| C12 | rs16925384 | 22807863 | A | 0.195 | 28302 | 0.0069 | 1.12 | 0.213 | 2394 | 0.0098 | 1.10 | 0.210 | 3046 |
| C12 | rs12425518 | 47206273 | A | 0.046 | 28295 | 0.031 | 1.17 | 0.054 | 2392 | 0.15 | 1.10 | 0.051 | 3042 |
| C12 | rs12581026 | 47223943 | C | 0.048 | 28310 | 0.0073 | 1.21 | 0.058 | 2394 | 0.059 | 1.13 | 0.054 | 3046 |
| C12 | rs784563 | 52152886 | G | 0.445 | 28308 | 0.049 | 1.07 | 0.461 | 2395 | 0.12 | 1.05 | 0.457 | 3047 |
| C12 | rs6580960 | 52445647 | A | 0.544 | 28320 | 0.068 | 1.06 | 0.558 | 2395 | 0.06 | 1.06 | 0.557 | 3047 |
| C12 | rs10878126 | 62930978 | G | 0.495 | 28299 | 0.016 | 1.08 | 0.515 | 2395 | 0.017 | 1.07 | 0.513 | 3046 |
| C12 | rs1465026 | 65789635 | G | 0.837 | 28309 | 0.011 | 1.12 | 0.852 | 2395 | 0.024 | 1.10 | 0.849 | 3047 |
| C12 | rs1526839 | 65801361 | G | 0.837 | 28240 | 0.019 | 1.11 | 0.851 | 2392 | 0.037 | 1.09 | 0.848 | 3042 |
| C12 | rs11835794 | 65812323 | T | 0.858 | 28321 | 0.056 | 1.10 | 0.868 | 2395 | 0.095 | 1.07 | 0.866 | 3047 |
| C12 | rs1470383 | 67493429 | C | 0.144 | 28320 | 0.0068 | 1.13 | 0.160 | 2395 | 0.0065 | 1.12 | 0.159 | 3047 |
| C12 | rs2584019 | 69318052 | T | 0.889 | 11415 | 0.00091 | 1.24 | 0.909 | 1775 | 0.0015 | 1.21 | 0.907 | 2094 |
| C12 | rs7978668 | 69325443 | T | 0.917 | 28319 | 0.0043 | 1.19 | 0.929 | 2395 | 0.0082 | 1.16 | 0.927 | 3047 |
| C12 | rs767760 | 69326403 | A | 0.914 | 28320 | 0.0041 | 1.19 | 0.927 | 2395 | 0.0068 | 1.16 | 0.925 | 3047 |
| C12 | rs7309888 | 69340682 | C | 0.794 | 28317 | 0.0034 | 1.13 | 0.813 | 2395 | 0.00095 | 1.13 | 0.814 | 3047 |
| C12 | rs7976576 | 69347673 | T | 0.795 | 28318 | 0.0049 | 1.12 | 0.813 | 2395 | 0.0015 | 1.13 | 0.813 | 3046 |
| C12 | rs949664 | 69368222 | A | 0.860 | 28228 | 0.0019 | 1.16 | 0.878 | 2393 | 0.0015 | 1.15 | 0.876 | 3043 |
| C12 | rs7135414 | 75996159 | A | 0.917 | 28317 | 0.0074 | 1.18 | 0.928 | 2395 | 0.0029 | 1.18 | 0.928 | 3047 |
| C12 | rs1401875 | 87986337 | T | 0.058 | 28291 | 0.095 | 1.12 | 0.064 | 2392 | 0.056 | 1.12 | 0.065 | 3041 |
| C12 | rs1568383 | 88017285 | T | 0.098 | 28316 | 0.008 | 1.15 | 0.111 | 2395 | 0.0049 | 1.14 | 0.110 | 3045 |
| C12 | rs11108788 | 95989532 | G | 0.036 | 27597 | 0.0085 | 1.24 | 0.044 | 2388 | 0.035 | 1.17 | 0.041 | 3040 |
| C12 | rs989264 | 96044433 | A | 0.044 | 28316 | 0.14 | 1.12 | 0.049 | 2395 | 0.18 | 1.10 | 0.049 | 3047 |
| C12 | rs7135604 | 96910191 | A | 0.657 | 28303 | 0.0022 | 1.11 | 0.680 | 2394 | 0.0076 | 1.09 | 0.676 | 3045 |
| C12 | rs864302 | 100490144 | T | 0.483 | 28309 | 0.033 | 1.07 | 0.501 | 2395 | 0.035 | 1.06 | 0.499 | 3047 |
| C12 | rs12311520 | 101693527 | C | 0.870 | 28247 | 0.09 | 1.09 | 0.879 | 2395 | 0.23 | 1.06 | 0.876 | 3046 |
| C12 | rs2247919 | 101699389 | T | 0.742 | 28307 | 0.071 | 1.07 | 0.755 | 2395 | 0.28 | 1.04 | 0.749 | 3046 |
| C12 | rs1015249 | 111609694 | C | 0.692 | 28316 | 0.0002 | 1.14 | 0.720 | 2395 | 0.00038 | 1.12 | 0.716 | 3046 |
| C12 | rs1293758 | 111917970 | A | 0.408 | 28316 | 0.0027 | 1.10 | 0.432 | 2394 | 0.0041 | 1.09 | 0.429 | 3046 |
| C12 | rs249047 | 112562237 | A | 0.614 | 28289 | 0.000091 | 1.14 | 0.645 | 2392 | 0.0015 | 1.10 | 0.637 | 3044 |
| C12 | rs1920947 | 114285453 | C | 0.810 | 28301 | 0.012 | 1.11 | 0.826 | 2394 | 0.014 | 1.10 | 0.824 | 3046 |
| C12 | rs816204 | 116492124 | A | 0.855 | 28175 | 0.0023 | 1.16 | 0.872 | 2384 | 0.0033 | 1.14 | 0.870 | 3034 |
| C12 | rs4767629 | 116739460 | T | 0.140 | 28316 | 0.025 | 1.11 | 0.153 | 2395 | 0.06 | 1.08 | 0.150 | 3047 |
| C12 | rs2592289 | 116759379 | A | 0.156 | 28311 | 0.0039 | 1.13 | 0.173 | 2395 | 0.0013 | 1.14 | 0.173 | 3047 |
| C12 | rs719450 | 119438416 | C | 0.918 | 28320 | 0.024 | 1.15 | 0.928 | 2394 | 0.019 | 1.14 | 0.928 | 3046 |
| C12 | rs3742049 | 119438873 | C | 0.919 | 28300 | 0.029 | 1.14 | 0.928 | 2394 | 0.023 | 1.13 | 0.928 | 3046 |
| C12 | rs1169081 | 120890295 | C | 0.693 | 28306 | 0.0032 | 1.11 | 0.715 | 2394 | 0.06 | 1.06 | 0.706 | 3045 |
| C12 | rs7979863 | 121629420 | A | 0.380 | 28309 | 0.022 | 1.08 | 0.399 | 2395 | 0.0025 | 1.10 | 0.402 | 3047 |
| C12 | rs10847227 | 121632638 | T | 0.279 | 28258 | 0.012 | 1.09 | 0.297 | 2395 | 0.00028 | 1.12 | 0.303 | 3047 |
| C12 | rs4765477 | 123287223 | T | 0.045 | 28300 | 0.016 | 1.20 | 0.053 | 2395 | 0.023 | 1.17 | 0.052 | 3047 |
| C12 | rs3759107 | 123548064 | T | 0.127 | 28320 | 0.023 | 1.11 | 0.139 | 2395 | 0.014 | 1.11 | 0.139 | 3047 |
| C12 | rs12304836 | 124654446 | C | 0.143 | 28205 | 0.00054 | 1.17 | 0.163 | 2385 | 0.00095 | 1.14 | 0.160 | 3034 |
| C12 | rs7310042 | 124675580 | G | 0.192 | 28317 | 0.018 | 1.10 | 0.207 | 2395 | 0.0043 | 1.11 | 0.209 | 3047 |
| C12 | rs470477 | 128010541 | T | 0.311 | 28306 | 0.0061 | 1.10 | 0.332 | 2395 | 0.013 | 1.08 | 0.328 | 3046 |
| C13 | rs4770252 | 21699475 | A | 0.806 | 28293 | 0.0088 | 1.12 | 0.822 | 2394 | 0.016 | 1.10 | 0.819 | 3044 |
| C13 | rs9285207 | 21722774 | C | 0.200 | 28256 | 0.0015 | 1.13 | 0.220 | 2387 | 0.0061 | 1.10 | 0.216 | 3037 |
| C13 | rs1887263 | 22381716 | A | 0.204 | 28175 | 0.067 | 1.08 | 0.216 | 2393 | 0.037 | 1.08 | 0.217 | 3042 |
| C13 | rs4769613 | 28036609 | T | 0.465 | 28313 | 0.00041 | 1.12 | 0.494 | 2394 | 0.00013 | 1.12 | 0.493 | 3046 |
| C13 | rs9566343 | 38000184 | T | 0.814 | 28309 | 0.011 | 1.11 | 0.830 | 2395 | 0.013 | 1.10 | 0.828 | 3046 |
| C13 | rs9315759 | 39616680 | C | 0.132 | 28308 | 0.0045 | 1.14 | 0.147 | 2394 | 0.0045 | 1.13 | 0.146 | 3046 |
| C13 | rs9525923 | 43681715 | A | 0.210 | 28320 | 0.00015 | 1.16 | 0.236 | 2395 | 0.0019 | 1.12 | 0.229 | 3047 |
| C13 | rs963569 | 44140379 | A | 0.753 | 28315 | 0.0074 | 1.11 | 0.771 | 2395 | 0.037 | 1.08 | 0.766 | 3047 |
| C13 | rs7322927 | 46552894 | A | 0.129 | 28318 | 0.021 | 1.12 | 0.142 | 2395 | 0.059 | 1.09 | 0.139 | 3047 |
| C13 | rs9534568 | 46693565 | A | 0.769 | 11414 | 0.086 | 1.08 | 0.783 | 1775 | 0.094 | 1.08 | 0.782 | 2094 |
| C13 | rs9526329 | 46866462 | G | 0.922 | 28290 | 0.00038 | 1.26 | 0.937 | 2395 | 0.0067 | 1.17 | 0.933 | 3047 |
| C13 | rs2050972 | 54308642 | C | 0.881 | 28309 | 0.01 | 1.14 | 0.894 | 2395 | 0.011 | 1.13 | 0.893 | 3047 |
| C13 | rs1993776 | 54406486 | C | 0.872 | 28309 | 0.011 | 1.14 | 0.885 | 2395 | 0.011 | 1.12 | 0.884 | 3047 |
| C13 | rs17070671 | 67943127 | T | 0.830 | 28318 | 0.00076 | 1.16 | 0.850 | 2395 | 0.017 | 1.10 | 0.843 | 3047 |
| C13 | rs1375719 | 102208783 | C | 0.499 | 28316 | 0.032 | 1.07 | 0.517 | 2394 | 0.12 | 1.05 | 0.511 | 3046 |
| C13 | rs2038709 | 102370990 | C | 0.084 | 28237 | 0.0084 | 1.16 | 0.096 | 2393 | 0.038 | 1.11 | 0.093 | 3045 |
| C13 | rs1347329 | 104032541 | C | 0.592 | 11392 | 0.005 | 1.12 | 0.619 | 1775 | 0.031 | 1.09 | 0.612 | 2093 |
| C13 | rs354438 | 105737486 | T | 0.355 | 28306 | 0.12 | 1.05 | 0.367 | 2395 | 0.33 | 1.03 | 0.362 | 3047 |
| C13 | rs1927771 | 106592839 | A | 0.416 | 28317 | 0.05 | 1.07 | 0.432 | 2395 | 0.078 | 1.05 | 0.429 | 3046 |
| C13 | rs1163852 | 110962872 | C | 0.713 | 28319 | 0.0031 | 1.11 | 0.735 | 2395 | 0.0056 | 1.10 | 0.732 | 3046 |
| C14 | rs10872856 | 20356515 | C | 0.914 | 28314 | 0.04 | 1.13 | 0.923 | 2395 | 0.022 | 1.13 | 0.923 | 3047 |
| C14 | rs991387 | 32384515 | G | 0.803 | 28319 | 0.026 | 1.10 | 0.818 | 2394 | 0.047 | 1.08 | 0.815 | 3046 |
| C14 | rs1950216 | 32385175 | T | 0.803 | 28318 | 0.027 | 1.10 | 0.818 | 2395 | 0.048 | 1.08 | 0.815 | 3047 |
| C14 | rs1742655 | 45622604 | C | 0.885 | 28321 | 0.0015 | 1.19 | 0.901 | 2395 | 0.0049 | 1.14 | 0.898 | 3047 |
| C14 | rs8007161 | 49042790 | T | 0.886 | 28278 | 0.027 | 1.12 | 0.898 | 2393 | 0.085 | 1.09 | 0.894 | 3044 |
| C14 | rs12436454 | 49076076 | T | 0.717 | 28226 | 0.0028 | 1.12 | 0.738 | 2393 | 0.012 | 1.09 | 0.733 | 3044 |
| C14 | rs4898630 | 49269010 | A | 0.878 | 28293 | 0.0079 | 1.15 | 0.892 | 2394 | 0.022 | 1.11 | 0.889 | 3046 |
| C14 | rs1458114 | 52093597 | T | 0.706 | 27829 | 0.0005 | 1.14 | 0.732 | 2338 | 0.0043 | 1.10 | 0.725 | 2979 |
| C14 | rs4444235 | 53480669 | T | 0.536 | 28305 | 0.37 | 1.03 | 0.543 | 2394 | 0.32 | 1.03 | 0.543 | 3046 |
| C14 | rs1957860 | 53499105 | T | 0.448 | 28313 | 0.27 | 0.97 | 0.439 | 2395 | 0.55 | 0.98 | 0.444 | 3047 |
| C14 | rs392030 | 58368609 | G | 0.636 | 28319 | 0.015 | 1.09 | 0.654 | 2395 | 0.029 | 1.07 | 0.651 | 3047 |
| C14 | rs2069334 | 58368941 | A | 0.188 | 28314 | 0.043 | 1.09 | 0.201 | 2395 | 0.099 | 1.06 | 0.197 | 3047 |
| C14 | rs17095237 | 58383406 | G | 0.164 | 28314 | 0.056 | 1.09 | 0.176 | 2395 | 0.17 | 1.06 | 0.172 | 3046 |
| C14 | rs10483707 | 58477558 | A | 0.141 | 28319 | 0.054 | 1.09 | 0.152 | 2395 | 0.14 | 1.06 | 0.148 | 3047 |
| C14 | rs11158238 | 58517378 | T | 0.142 | 28317 | 0.06 | 1.09 | 0.153 | 2395 | 0.11 | 1.07 | 0.150 | 3047 |
| C14 | rs7147624 | 64935378 | G | 0.801 | 28311 | 0.011 | 1.11 | 0.817 | 2395 | 0.034 | 1.08 | 0.813 | 3046 |
| C14 | rs2411822 | 64948148 | C | 0.486 | 28314 | 0.024 | 1.08 | 0.505 | 2395 | 0.11 | 1.05 | 0.498 | 3047 |
| C14 | rs1953416 | 64948560 | T | 0.483 | 28267 | 0.016 | 1.08 | 0.503 | 2391 | 0.073 | 1.05 | 0.497 | 3042 |
| C14 | rs7145500 | 64953965 | A | 0.663 | 28319 | 0.031 | 1.08 | 0.679 | 2395 | 0.05 | 1.06 | 0.676 | 3047 |
| C14 | rs12433694 | 65380138 | C | 0.483 | 28319 | 0.031 | 1.07 | 0.500 | 2395 | 0.1 | 1.05 | 0.494 | 3047 |
| C14 | rs1147443 | 65538514 | T | 0.251 | 28316 | 0.000014 | 1.17 | 0.282 | 2395 | 0.0000074 | 1.16 | 0.280 | 3047 |
| C14 | rs1147437 | 65544105 | G | 0.380 | 28316 | 0.000065 | 1.14 | 0.412 | 2395 | 0.000058 | 1.13 | 0.409 | 3046 |
| C14 | rs894921 | 65546005 | G | 0.376 | 28318 | 0.000056 | 1.14 | 0.408 | 2395 | 0.000054 | 1.13 | 0.405 | 3047 |
| C14 | rs6573648 | 65617378 | G | 0.272 | 28316 | 0.000041 | 1.16 | 0.302 | 2395 | 0.000028 | 1.15 | 0.300 | 3047 |
| C14 | rs17754686 | 65667738 | A | 0.307 | 28239 | 0.00015 | 1.14 | 0.335 | 2392 | 0.00007 | 1.13 | 0.334 | 3044 |
| C14 | rs8003722 | 68388015 | A | 0.106 | 28317 | 0.0085 | 1.14 | 0.119 | 2394 | 0.024 | 1.11 | 0.116 | 3046 |
| C14 | rs1291302 | 69449823 | C | 0.265 | 28043 | 0.13 | 1.06 | 0.276 | 2363 | 0.22 | 1.04 | 0.273 | 3015 |
| C14 | rs10498639 | 93845279 | A | 0.428 | 28316 | 0.0088 | 1.09 | 0.449 | 2395 | 0.018 | 1.07 | 0.445 | 3047 |
| C14 | rs2281519 | 93846385 | T | 0.260 | 28281 | 0.0023 | 1.12 | 0.282 | 2395 | 0.014 | 1.09 | 0.276 | 3045 |
| C14 | rs2895811 | 99203695 | C | 0.449 | 28199 | 0.0018 | 1.11 | 0.474 | 2385 | 0.0015 | 1.10 | 0.472 | 3033 |
| C14 | rs7158073 | 99211442 | T | 0.430 | 28102 | 0.0005 | 1.12 | 0.458 | 2379 | 0.0016 | 1.10 | 0.453 | 3027 |
| C14 | rs12435918 | 99240648 | G | 0.333 | 28318 | 0.0006 | 1.12 | 0.359 | 2395 | 0.0016 | 1.10 | 0.355 | 3047 |
| C14 | rs2273840 | 99257897 | A | 0.931 | 28124 | 0.0031 | 1.22 | 0.943 | 2369 | 0.0035 | 1.20 | 0.941 | 3019 |
| C14 | rs17776453 | 100774793 | G | 0.955 | 28313 | 0.095 | 1.15 | 0.961 | 2394 | 0.033 | 1.17 | 0.962 | 3045 |
| C14 | rs4708 | 102469201 | A | 0.549 | 28293 | 0.007 | 1.09 | 0.571 | 2393 | 0.019 | 1.07 | 0.566 | 3043 |
| C14 | rs10129841 | 102473912 | A | 0.578 | 28316 | 0.0014 | 1.11 | 0.604 | 2395 | 0.012 | 1.08 | 0.596 | 3047 |
| C14 | rs8016595 | 103791279 | C | 0.756 | 28304 | 0.016 | 1.10 | 0.773 | 2395 | 0.04 | 1.07 | 0.769 | 3047 |
| C15 | rs4572353 | 24287601 | T | 0.498 | 28314 | 0.0003 | 1.13 | 0.528 | 2393 | 0.00013 | 1.12 | 0.526 | 3043 |
| C15 | rs2110209 | 24946433 | A | 0.048 | 28318 | 0.00051 | 1.28 | 0.061 | 2395 | 0.0061 | 1.20 | 0.057 | 3047 |
| C15 | rs4779984 | 30302218 | G | 0.100 | 28236 | 0.0015 | 1.18 | 0.116 | 2392 | 0.003 | 1.15 | 0.114 | 3043 |
| C15 | rs7183966 | 34836718 | G | 0.129 | 28307 | 0.00037 | 1.18 | 0.149 | 2393 | 0.0032 | 1.13 | 0.144 | 3044 |
| C15 | rs1901725 | 34837201 | T | 0.129 | 28318 | 0.00038 | 1.18 | 0.149 | 2395 | 0.0034 | 1.13 | 0.144 | 3047 |
| C15 | rs1194620 | 34902036 | T | 0.233 | 28268 | 0.0084 | 1.11 | 0.251 | 2394 | 0.042 | 1.07 | 0.246 | 3044 |
| C15 | rs1841441 | 37167818 | A | 0.471 | 28308 | 0.021 | 1.08 | 0.489 | 2393 | 0.0055 | 1.09 | 0.491 | 3045 |
| C15 | rs7172161 | 43778072 | G | 0.517 | 28267 | 0.067 | 1.06 | 0.532 | 2393 | 0.045 | 1.06 | 0.532 | 3045 |
| C15 | rs11630033 | 43850557 | C | 0.902 | 28204 | 0.065 | 1.11 | 0.911 | 2389 | 0.1 | 1.09 | 0.909 | 3040 |
| C15 | rs1156234 | 51156742 | G | 0.795 | 28314 | 0.0028 | 1.13 | 0.815 | 2395 | 0.0077 | 1.10 | 0.811 | 3047 |
| C15 | rs2553223 | 52657225 | G | 0.344 | 28301 | 0.000086 | 1.14 | 0.375 | 2395 | 0.00015 | 1.12 | 0.371 | 3047 |
| C15 | rs2414325 | 52675829 | T | 0.489 | 28318 | 0.00012 | 1.13 | 0.520 | 2395 | 0.00022 | 1.12 | 0.516 | 3047 |
| C15 | rs8041044 | 53452605 | C | 0.915 | 28200 | 0.015 | 1.16 | 0.925 | 2389 | 0.044 | 1.12 | 0.923 | 3039 |
| C15 | rs4775234 | 58117938 | C | 0.259 | 27735 | 0.0047 | 1.11 | 0.280 | 2354 | 0.031 | 1.08 | 0.273 | 2995 |
| C15 | rs11072793 | 76793497 | G | 0.231 | 28268 | 0.00025 | 1.15 | 0.257 | 2388 | 0.0013 | 1.12 | 0.251 | 3040 |
| C15 | rs4255747 | 77512872 | G | 0.677 | 28311 | 0.07 | 1.07 | 0.691 | 2395 | 0.24 | 1.04 | 0.685 | 3047 |
| C15 | rs4887480 | 84366589 | G | 0.771 | 28306 | 0.0014 | 1.13 | 0.793 | 2395 | 0.0051 | 1.11 | 0.788 | 3045 |
| C15 | rs12595195 | 88001739 | C | 0.280 | 28292 | 0.0093 | 1.10 | 0.299 | 2395 | 0.056 | 1.06 | 0.292 | 3046 |
| C15 | rs3743369 | 90508573 | T | 0.616 | 28313 | 0.032 | 1.07 | 0.633 | 2394 | 0.029 | 1.07 | 0.632 | 3046 |
| C15 | rs6496899 | 90511768 | C | 0.766 | 28152 | 0.02 | 1.10 | 0.781 | 2375 | 0.014 | 1.09 | 0.781 | 3024 |
| C15 | rs10152962 | 92112175 | G | 0.153 | 28319 | 0.014 | 1.11 | 0.168 | 2393 | 0.0078 | 1.11 | 0.167 | 3045 |
| C15 | rs176656 | 93537286 | A | 0.093 | 28317 | 0.054 | 1.11 | 0.103 | 2395 | 0.055 | 1.10 | 0.102 | 3047 |
| C15 | rs7177625 | 95764563 | G | 0.687 | 28300 | 0.029 | 1.08 | 0.703 | 2395 | 0.16 | 1.05 | 0.696 | 3046 |
| C15 | rs2684790 | 97309063 | T | 0.146 | 28314 | 0.0036 | 1.14 | 0.163 | 2395 | 0.011 | 1.11 | 0.160 | 3047 |
| C16 | rs8057913 | 2009168 | C | 0.266 | 28244 | 0.0066 | 1.10 | 0.286 | 2391 | 0.0057 | 1.10 | 0.285 | 3042 |
| C16 | rs2286472 | 2010569 | C | 0.271 | 28249 | 0.011 | 1.10 | 0.290 | 2385 | 0.007 | 1.09 | 0.289 | 3036 |
| C16 | rs8045288 | 2013121 | T | 0.269 | 28279 | 0.01 | 1.10 | 0.287 | 2394 | 0.0082 | 1.09 | 0.286 | 3045 |
| C16 | rs3785284 | 2014037 | C | 0.267 | 28138 | 0.012 | 1.10 | 0.285 | 2384 | 0.009 | 1.09 | 0.284 | 3032 |
| C16 | rs8053779 | 18979415 | G | 0.738 | 28314 | 0.12 | 1.06 | 0.749 | 2395 | 0.13 | 1.05 | 0.748 | 3047 |
| C16 | rs7500550 | 18985697 | G | 0.782 | 28319 | 0.065 | 1.08 | 0.794 | 2394 | 0.045 | 1.08 | 0.794 | 3046 |
| C16 | rs1634675 | 19801122 | G | 0.789 | 28308 | 0.023 | 1.10 | 0.804 | 2395 | 0.054 | 1.07 | 0.800 | 3047 |
| C16 | rs8050136 | 52373776 | A | 0.404 | 28316 | 0.0046 | 1.10 | 0.426 | 2394 | 0.00029 | 1.11 | 0.430 | 3046 |
| C16 | rs3751812 | 52375961 | T | 0.403 | 28246 | 0.0041 | 1.10 | 0.426 | 2392 | 0.00024 | 1.12 | 0.430 | 3044 |
| C16 | rs1033043 | 52774858 | T | 0.577 | 28311 | 0.027 | 1.08 | 0.594 | 2394 | 0.052 | 1.06 | 0.591 | 3046 |
| C16 | rs2665282 | 52779886 | A | 0.577 | 28267 | 0.029 | 1.07 | 0.594 | 2391 | 0.059 | 1.06 | 0.591 | 3041 |
| C16 | rs12924635 | 60072235 | G | 0.397 | 27765 | 0.0043 | 1.10 | 0.420 | 2306 | 0.023 | 1.07 | 0.414 | 2944 |
| C16 | rs2962080 | 61410292 | G | 0.609 | 28310 | 0.0056 | 1.10 | 0.631 | 2394 | 0.06 | 1.06 | 0.623 | 3046 |
| C16 | rs7192142 | 62724486 | T | 0.810 | 28254 | 0.00063 | 1.16 | 0.832 | 2385 | 0.0059 | 1.11 | 0.826 | 3036 |
| C16 | rs12927636 | 72310379 | G | 0.108 | 28296 | 0.0028 | 1.16 | 0.123 | 2393 | 0.0065 | 1.13 | 0.121 | 3045 |
| C16 | rs1038864 | 72320058 | C | 0.185 | 28319 | 0.012 | 1.11 | 0.201 | 2395 | 0.019 | 1.09 | 0.198 | 3047 |
| C16 | rs4243110 | 73810027 | T | 0.462 | 28299 | 0.01 | 1.09 | 0.482 | 2394 | 0.0043 | 1.09 | 0.482 | 3046 |
| C16 | rs1862737 | 73839465 | C | 0.484 | 28309 | 0.0017 | 1.11 | 0.509 | 2395 | 0.0013 | 1.10 | 0.508 | 3046 |
| C16 | rs7188879 | 78364890 | T | 0.966 | 28303 | 0.048 | 1.21 | 0.972 | 2394 | 0.11 | 1.14 | 0.970 | 3046 |
| C16 | rs1870843 | 81316815 | T | 0.602 | 28313 | 0.0081 | 1.09 | 0.623 | 2395 | 0.0098 | 1.08 | 0.621 | 3047 |
| C16 | rs10514560 | 81328576 | C | 0.734 | 28315 | 0.011 | 1.10 | 0.752 | 2395 | 0.022 | 1.08 | 0.749 | 3047 |
| C16 | rs4075964 | 86985334 | A | 0.283 | 28229 | 0.0063 | 1.10 | 0.304 | 2392 | 0.016 | 1.08 | 0.299 | 3042 |
| C16 | rs904800 | 87044441 | G | 0.383 | 28296 | 0.0057 | 1.10 | 0.405 | 2395 | 0.0062 | 1.09 | 0.403 | 3045 |
| C17 | rs4130140 | 257873 | A | 0.174 | 28319 | 0.00063 | 1.15 | 0.195 | 2395 | 0.0027 | 1.12 | 0.191 | 3047 |
| C17 | rs4791759 | 7618652 | A | 0.041 | 28305 | 0.065 | 1.16 | 0.047 | 2394 | 0.32 | 1.07 | 0.044 | 3044 |
| C17 | rs3891720 | 9331114 | C | 0.872 | 28298 | 0.0034 | 1.16 | 0.888 | 2392 | 0.026 | 1.11 | 0.883 | 3044 |
| C17 | rs9903582 | 10175559 | C | 0.749 | 28317 | 0.01 | 1.10 | 0.767 | 2395 | 0.022 | 1.08 | 0.764 | 3047 |
| C17 | rs12449610 | 13998813 | T | 0.669 | 28319 | 0.0025 | 1.11 | 0.692 | 2395 | 0.0087 | 1.09 | 0.687 | 3047 |
| C17 | rs2323095 | 14000644 | A | 0.664 | 28312 | 0.0027 | 1.11 | 0.687 | 2395 | 0.013 | 1.08 | 0.681 | 3047 |
| C17 | rs2856177 | 14041754 | A | 0.622 | 28308 | 0.0085 | 1.09 | 0.642 | 2395 | 0.016 | 1.08 | 0.639 | 3047 |
| C17 | rs4401079 | 16755421 | T | 0.211 | 27570 | 0.028 | 1.09 | 0.226 | 2340 | 0.015 | 1.09 | 0.226 | 2975 |
| C17 | rs4925108 | 17590148 | C | 0.394 | 28297 | 0.0015 | 1.11 | 0.420 | 2394 | 0.014 | 1.08 | 0.412 | 3046 |
| C17 | rs752579 | 17601072 | G | 0.390 | 28286 | 0.0051 | 1.10 | 0.412 | 2394 | 0.043 | 1.06 | 0.404 | 3046 |
| C17 | rs4925109 | 17602527 | A | 0.318 | 28316 | 0.00032 | 1.13 | 0.345 | 2395 | 0.0045 | 1.09 | 0.337 | 3047 |
| C17 | rs11649804 | 17637480 | A | 0.292 | 28148 | 0.00029 | 1.14 | 0.319 | 2341 | 0.0041 | 1.10 | 0.312 | 2987 |
| C17 | rs4925114 | 17651995 | A | 0.357 | 28266 | 0.000045 | 1.15 | 0.389 | 2391 | 0.0017 | 1.10 | 0.379 | 3042 |
| C17 | rs11868035 | 17655826 | A | 0.294 | 28278 | 0.000068 | 1.15 | 0.324 | 2393 | 0.0016 | 1.11 | 0.315 | 3043 |
| C17 | rs9902941 | 17674485 | C | 0.347 | 28319 | 0.0000029 | 1.17 | 0.384 | 2395 | 0.00095 | 1.11 | 0.371 | 3047 |
| C17 | rs4925119 | 17683629 | A | 0.345 | 28218 | 0.0000077 | 1.16 | 0.380 | 2387 | 0.0021 | 1.10 | 0.367 | 3036 |
| C17 | rs3183702 | 17688014 | T | 0.348 | 28108 | 0.0000041 | 1.17 | 0.384 | 2388 | 0.0013 | 1.10 | 0.371 | 3038 |
| C17 | rs4925125 | 17735169 | T | 0.353 | 28060 | 0.0000082 | 1.16 | 0.388 | 2374 | 0.0016 | 1.10 | 0.376 | 3021 |
| C17 | rs6502622 | 17775416 | G | 0.358 | 28303 | 0.0000065 | 1.16 | 0.393 | 2395 | 0.0019 | 1.10 | 0.380 | 3047 |
| C17 | rs2955355 | 17889200 | C | 0.285 | 28312 | 0.00000087 | 1.19 | 0.321 | 2395 | 0.00033 | 1.12 | 0.309 | 3047 |
| C17 | rs854813 | 17944570 | C | 0.385 | 28182 | 0.000011 | 1.16 | 0.420 | 2393 | 0.0031 | 1.09 | 0.406 | 3042 |
| C17 | rs854793 | 17971249 | A | 0.202 | 28313 | 0.000033 | 1.18 | 0.229 | 2395 | 0.0018 | 1.12 | 0.220 | 3047 |
| C17 | rs854787 | 17975744 | C | 0.400 | 28104 | 0.000046 | 1.14 | 0.432 | 2381 | 0.0046 | 1.09 | 0.420 | 3028 |
| C17 | rs854771 | 18000917 | C | 0.554 | 28297 | 0.00061 | 1.12 | 0.581 | 2395 | 0.00086 | 1.10 | 0.578 | 3046 |
| C17 | rs2746025 | 18092336 | G | 0.640 | 28315 | 0.0001 | 1.14 | 0.670 | 2394 | 0.0013 | 1.11 | 0.663 | 3046 |
| C17 | rs9911448 | 21030674 | C | 0.961 | 28319 | 0.023 | 1.23 | 0.968 | 2395 | 0.083 | 1.15 | 0.966 | 3047 |
| C17 | rs7210156 | 34250987 | G | 0.929 | 28256 | 0.0024 | 1.23 | 0.941 | 2392 | 0.019 | 1.15 | 0.937 | 3043 |
| C17 | rs1122326 | 37528399 | C | 0.242 | 28309 | 0.00086 | 1.13 | 0.265 | 2395 | 0.02 | 1.08 | 0.257 | 3047 |
| C17 | rs12603327 | 37544078 | C | 0.242 | 28318 | 0.00093 | 1.13 | 0.265 | 2395 | 0.021 | 1.08 | 0.256 | 3047 |
| C17 | rs7214921 | 37547551 | C | 0.242 | 28291 | 0.00095 | 1.13 | 0.265 | 2394 | 0.021 | 1.08 | 0.256 | 3045 |
| C17 | rs4794128 | 45727464 | A | 0.276 | 28309 | 0.0061 | 1.10 | 0.296 | 2395 | 0.0081 | 1.09 | 0.293 | 3047 |
| C17 | rs11656018 | 48969375 | C | 0.707 | 28240 | 0.0012 | 1.13 | 0.731 | 2394 | 0.002 | 1.11 | 0.728 | 3045 |
| C17 | rs9895713 | 50752149 | G | 0.178 | 28297 | 0.013 | 1.11 | 0.194 | 2395 | 0.03 | 1.09 | 0.191 | 3047 |
| C17 | rs1985961 | 59470994 | C | 0.413 | 28311 | 0.014 | 1.09 | 0.433 | 2395 | 0.0086 | 1.08 | 0.432 | 3047 |
| C17 | rs312750 | 65855134 | C | 0.548 | 28314 | 0.0018 | 1.11 | 0.573 | 2395 | 0.019 | 1.07 | 0.565 | 3047 |
| C17 | rs9911654 | 67172007 | T | 0.899 | 28314 | 0.0099 | 1.16 | 0.912 | 2395 | 0.037 | 1.11 | 0.908 | 3047 |
| C17 | rs2041088 | 67702720 | A | 0.722 | 28312 | 0.0041 | 1.11 | 0.743 | 2395 | 0.0028 | 1.11 | 0.741 | 3047 |
| C17 | rs2159010 | 67708572 | A | 0.730 | 28262 | 0.0097 | 1.10 | 0.748 | 2395 | 0.0053 | 1.10 | 0.748 | 3045 |
| C17 | rs191227 | 69202771 | A | 0.579 | 28300 | 0.022 | 1.08 | 0.597 | 2394 | 0.043 | 1.06 | 0.594 | 3046 |
| C17 | rs1000299 | 70660148 | A | 0.055 | 28315 | 0.0044 | 1.21 | 0.066 | 2394 | 0.015 | 1.16 | 0.063 | 3046 |
| C17 | rs17674253 | 74732189 | A | 0.583 | 28311 | 0.032 | 1.07 | 0.600 | 2393 | 0.071 | 1.06 | 0.596 | 3045 |
| C18 | rs1791098 | 3294629 | C | 0.763 | 28318 | 0.022 | 1.09 | 0.779 | 2395 | 0.24 | 1.04 | 0.770 | 3047 |
| C18 | rs12964045 | 13442635 | C | 0.131 | 28312 | 0.015 | 1.12 | 0.145 | 2394 | 0.031 | 1.10 | 0.142 | 3046 |
| C18 | rs4271662 | 26051291 | C | 0.587 | 28256 | 0.061 | 1.06 | 0.602 | 2390 | 0.092 | 1.05 | 0.599 | 3039 |
| C18 | rs12964741 | 35159625 | A | 0.668 | 28119 | 0.0049 | 1.10 | 0.689 | 2359 | 0.0013 | 1.11 | 0.690 | 3008 |
| C18 | rs1015735 | 39877321 | C | 0.267 | 28220 | 0.035 | 1.08 | 0.283 | 2387 | 0.15 | 1.05 | 0.277 | 3037 |
| C18 | rs4340393 | 39884052 | T | 0.266 | 28320 | 0.033 | 1.08 | 0.282 | 2395 | 0.13 | 1.05 | 0.276 | 3047 |
| C18 | rs17734724 | 43268749 | G | 0.276 | 28318 | 0.00071 | 1.13 | 0.300 | 2395 | 0.0024 | 1.10 | 0.296 | 3047 |
| C18 | rs1790778 | 45715039 | G | 0.512 | 28314 | 0.00027 | 1.13 | 0.541 | 2394 | 0.000024 | 1.13 | 0.543 | 3046 |
| C18 | rs1557359 | 45734896 | G | 0.408 | 28317 | 0.00063 | 1.12 | 0.435 | 2395 | 0.00016 | 1.12 | 0.435 | 3047 |
| C18 | rs634285 | 53273824 | G | 0.913 | 11415 | 0.00075 | 1.28 | 0.930 | 1775 | 0.0036 | 1.22 | 0.927 | 2094 |
| C18 | rs580647 | 53284831 | A | 0.886 | 28267 | 0.034 | 1.12 | 0.897 | 2394 | 0.057 | 1.09 | 0.895 | 3045 |
| C18 | rs4940582 | 53743264 | G | 0.851 | 28314 | 0.0016 | 1.16 | 0.869 | 2394 | 0.0016 | 1.14 | 0.867 | 3046 |
| C18 | rs657773 | 55097571 | C | 0.556 | 28318 | 0.00062 | 1.12 | 0.583 | 2395 | 0.012 | 1.08 | 0.574 | 3047 |
| C18 | rs4806 | 55146376 | C | 0.285 | 28175 | 0.016 | 1.09 | 0.303 | 2376 | 0.049 | 1.07 | 0.299 | 3025 |
| C18 | rs9319948 | 55193376 | A | 0.285 | 28174 | 0.025 | 1.08 | 0.302 | 2387 | 0.074 | 1.06 | 0.297 | 3033 |
| C18 | rs8088514 | 55213078 | C | 0.461 | 28276 | 0.0093 | 1.09 | 0.482 | 2386 | 0.037 | 1.06 | 0.476 | 3038 |
| C18 | rs8082768 | 55792708 | T | 0.182 | 28281 | 0.007 | 1.12 | 0.200 | 2390 | 0.01 | 1.10 | 0.197 | 3040 |
| C18 | rs9948303 | 55911746 | G | 0.102 | 28316 | 0.084 | 1.10 | 0.110 | 2395 | 0.0079 | 1.13 | 0.114 | 3047 |
| C18 | rs603119 | 56407631 | T | 0.391 | 28278 | 0.072 | 1.06 | 0.406 | 2394 | 0.021 | 1.07 | 0.408 | 3045 |
| C18 | rs2332026 | 56572162 | A | 0.505 | 28318 | 0.059 | 1.06 | 0.520 | 2395 | 0.051 | 1.06 | 0.519 | 3047 |
| C18 | rs3744927 | 58396227 | G | 0.772 | 28317 | 0.0024 | 1.13 | 0.793 | 2395 | 0.017 | 1.09 | 0.787 | 3047 |
| C18 | rs2332575 | 58449327 | A | 0.054 | 28302 | 0.014 | 1.18 | 0.064 | 2392 | 0.028 | 1.15 | 0.062 | 3044 |
| C18 | rs7241455 | 58476248 | T | 0.063 | 28318 | 0.0045 | 1.20 | 0.075 | 2395 | 0.0039 | 1.18 | 0.074 | 3047 |
| C18 | rs12458349 | 58505190 | G | 0.061 | 28155 | 0.0014 | 1.23 | 0.074 | 2389 | 0.0013 | 1.21 | 0.073 | 3036 |
| C18 | rs1365254 | 70238283 | T | 0.196 | 28319 | 0.066 | 1.08 | 0.208 | 2395 | 0.036 | 1.08 | 0.209 | 3047 |
| C18 | rs4892210 | 70241069 | A | 0.196 | 28316 | 0.057 | 1.08 | 0.209 | 2395 | 0.031 | 1.08 | 0.209 | 3047 |
| C18 | rs2850889 | 73087595 | C | 0.409 | 28316 | 0.00013 | 1.13 | 0.440 | 2395 | 0.00015 | 1.12 | 0.437 | 3046 |
| C18 | rs2156643 | 73088668 | G | 0.409 | 28304 | 0.00013 | 1.13 | 0.440 | 2395 | 0.00015 | 1.12 | 0.436 | 3047 |
| C18 | rs2850892 | 73088829 | C | 0.409 | 28292 | 0.00014 | 1.13 | 0.440 | 2393 | 0.00016 | 1.12 | 0.436 | 3043 |
| C19 | rs10414677 | 330003 | T | 0.285 | 27883 | 0.048 | 1.07 | 0.299 | 2322 | 0.27 | 1.04 | 0.292 | 2956 |
| C19 | rs758503 | 446722 | T | 0.302 | 27981 | 0.012 | 1.09 | 0.321 | 2371 | 0.011 | 1.08 | 0.319 | 3018 |
| C19 | rs4740 | 4187996 | A | 0.274 | 28312 | 0.017 | 1.09 | 0.291 | 2395 | 0.054 | 1.07 | 0.286 | 3047 |
| C19 | rs1045750 | 4202069 | C | 0.269 | 28318 | 0.021 | 1.09 | 0.286 | 2394 | 0.054 | 1.07 | 0.281 | 3046 |
| C19 | rs4807567 | 4229502 | T | 0.427 | 28263 | 0.07 | 1.06 | 0.442 | 2394 | 0.07 | 1.06 | 0.441 | 3045 |
| C19 | rs10419363 | 4231186 | T | 0.306 | 28279 | 0.076 | 1.06 | 0.319 | 2390 | 0.069 | 1.06 | 0.318 | 3040 |
| C19 | rs10406508 | 8795907 | T | 0.764 | 11415 | 0.0031 | 1.15 | 0.788 | 1775 | 0.0092 | 1.12 | 0.784 | 2094 |
| C19 | rs7248277 | 14236895 | C | 0.570 | 28306 | 0.0065 | 1.09 | 0.592 | 2394 | 0.0034 | 1.09 | 0.591 | 3045 |
| C19 | rs2305758 | 17024661 | G | 0.691 | 28308 | 0.0044 | 1.11 | 0.712 | 2394 | 0.0026 | 1.10 | 0.711 | 3046 |
| C19 | rs2278997 | 17027981 | T | 0.229 | 28315 | 0.0000018 | 1.20 | 0.262 | 2395 | 0.0000088 | 1.16 | 0.257 | 3047 |
| C19 | rs12459084 | 17031885 | T | 0.350 | 27962 | 0.00013 | 1.14 | 0.380 | 2387 | 0.002 | 1.10 | 0.372 | 3037 |
| C19 | rs7254154 | 17039119 | C | 0.352 | 28271 | 0.00019 | 1.13 | 0.381 | 2389 | 0.0028 | 1.10 | 0.373 | 3041 |
| C19 | rs7246865 | 17080105 | A | 0.205 | 28321 | 0.00016 | 1.16 | 0.230 | 2395 | 0.00025 | 1.14 | 0.227 | 3047 |
| C19 | rs4933027 | 22695812 | T | 0.083 | 28318 | 0.00019 | 1.23 | 0.101 | 2395 | 0.0011 | 1.18 | 0.097 | 3047 |
| C19 | rs4805463 | 34828875 | G | 0.909 | 28316 | 0.0018 | 1.20 | 0.923 | 2395 | 0.0073 | 1.15 | 0.920 | 3047 |
| C19 | rs346535 | 48943265 | T | 0.234 | 28317 | 0.0031 | 1.12 | 0.255 | 2395 | 0.00078 | 1.12 | 0.255 | 3047 |
| C19 | rs377702 | 50054507 | T | 0.391 | 28304 | 0.007 | 1.09 | 0.412 | 2395 | 0.014 | 1.08 | 0.409 | 3047 |
| C19 | rs3760802 | 53768499 | A | 0.505 | 28309 | 0.073 | 1.06 | 0.519 | 2392 | 0.13 | 1.05 | 0.516 | 3044 |
| C19 | rs11668495 | 58638423 | C | 0.940 | 28270 | 0.065 | 1.14 | 0.947 | 2392 | 0.12 | 1.11 | 0.945 | 3044 |
| C19 | rs8113561 | 62070079 | G | 0.954 | 28311 | 0.0055 | 1.26 | 0.963 | 2395 | 0.025 | 1.18 | 0.960 | 3047 |
| C20 | rs723477 | 237362 | T | 0.742 | 28284 | 0.004 | 1.12 | 0.762 | 2389 | 0.0029 | 1.11 | 0.761 | 3040 |
| C20 | rs1741285 | 4064436 | G | 0.560 | 28300 | 0.00075 | 1.12 | 0.588 | 2392 | 0.00072 | 1.11 | 0.585 | 3043 |
| C20 | rs6084644 | 4069799 | G | 0.437 | 28319 | 0.00096 | 1.11 | 0.464 | 2395 | 0.00062 | 1.11 | 0.462 | 3047 |
| C20 | rs6076623 | 4084671 | T | 0.367 | 28302 | 0.000045 | 1.15 | 0.399 | 2395 | 0.0000036 | 1.15 | 0.400 | 3047 |
| C20 | rs1741315 | 4103948 | A | 0.484 | 28317 | 0.0014 | 1.11 | 0.510 | 2395 | 0.0012 | 1.10 | 0.508 | 3047 |
| C20 | rs1741318 | 4105600 | A | 0.450 | 28315 | 0.0025 | 1.10 | 0.474 | 2394 | 0.0013 | 1.10 | 0.473 | 3046 |
| C20 | rs236110 | 5881108 | G | 0.871 | 28299 | 0.00079 | 1.19 | 0.889 | 2393 | 0.0036 | 1.14 | 0.885 | 3044 |
| C20 | rs236113 | 5882566 | T | 0.883 | 28321 | 0.001 | 1.19 | 0.900 | 2395 | 0.0063 | 1.14 | 0.896 | 3047 |
| C20 | rs2026058 | 13639900 | T | 0.768 | 28319 | 0.048 | 1.08 | 0.781 | 2395 | 0.084 | 1.06 | 0.779 | 3047 |
| C20 | rs6079235 | 13886789 | C | 0.736 | 28308 | 0.096 | 1.06 | 0.748 | 2394 | 0.12 | 1.05 | 0.746 | 3046 |
| C20 | rs411944 | 15006882 | G | 0.651 | 28275 | 0.0033 | 1.11 | 0.673 | 2392 | 0.035 | 1.07 | 0.665 | 3044 |
| C20 | rs6034134 | 15182479 | G | 0.596 | 28318 | 0.026 | 1.08 | 0.614 | 2395 | 0.065 | 1.06 | 0.610 | 3047 |
| C20 | rs199812 | 22264552 | C | 0.888 | 28288 | 0.0074 | 1.15 | 0.902 | 2394 | 0.0026 | 1.16 | 0.902 | 3046 |
| C20 | rs734484 | 31943651 | C | 0.713 | 28316 | 0.079 | 1.07 | 0.726 | 2394 | 0.1 | 1.06 | 0.724 | 3045 |
| C20 | rs981303 | 45947245 | A | 0.931 | 28301 | 0.057 | 1.14 | 0.939 | 2391 | 0.011 | 1.17 | 0.941 | 3043 |
| C20 | rs4811008 | 48277796 | G | 0.463 | 27959 | 0.00032 | 1.13 | 0.493 | 2348 | 0.0065 | 1.09 | 0.483 | 2993 |
| C20 | rs913670 | 48339923 | C | 0.634 | 28276 | 0.00081 | 1.12 | 0.660 | 2395 | 0.0051 | 1.09 | 0.654 | 3046 |
| C20 | rs913673 | 48341447 | C | 0.541 | 28304 | 0.00017 | 1.13 | 0.571 | 2395 | 0.0029 | 1.09 | 0.563 | 3047 |
| C20 | rs6068930 | 52700213 | G | 0.040 | 28199 | 0.013 | 1.22 | 0.048 | 2389 | 0.021 | 1.18 | 0.046 | 3039 |
| C20 | rs486921 | 56349782 | C | 0.854 | 28319 | 0.0042 | 1.15 | 0.870 | 2394 | 0.013 | 1.11 | 0.867 | 3046 |
| C20 | rs6128327 | 56375963 | A | 0.647 | 27522 | 0.029 | 1.08 | 0.664 | 2355 | 0.079 | 1.06 | 0.660 | 2992 |
| C20 | rs6128397 | 56686538 | G | 0.690 | 28315 | 0.012 | 1.09 | 0.708 | 2395 | 0.031 | 1.07 | 0.704 | 3047 |
| C20 | rs6128843 | 58519280 | T | 0.183 | 28319 | 0.00057 | 1.15 | 0.205 | 2395 | 0.0027 | 1.12 | 0.201 | 3047 |
| C21 | rs2822391 | 14334270 | A | 0.553 | 28310 | 0.021 | 1.08 | 0.572 | 2394 | 0.0088 | 1.08 | 0.572 | 3045 |
| C21 | rs2822744 | 14821123 | A | 0.158 | 28318 | 0.026 | 1.10 | 0.171 | 2395 | 0.045 | 1.08 | 0.169 | 3047 |
| C21 | rs2822753 | 14824849 | G | 0.356 | 28297 | 0.057 | 1.07 | 0.371 | 2394 | 0.13 | 1.05 | 0.367 | 3046 |
| C21 | rs1349227 | 16738666 | T | 0.242 | 28301 | 0.018 | 1.09 | 0.258 | 2393 | 0.021 | 1.08 | 0.256 | 3045 |
| C21 | rs2017705 | 21803122 | C | 0.074 | 28287 | 0.0091 | 1.17 | 0.085 | 2390 | 0.058 | 1.11 | 0.081 | 3041 |
| C21 | rs2828495 | 24038081 | A | 0.212 | 28316 | 0.001 | 1.14 | 0.234 | 2395 | 0.00089 | 1.12 | 0.232 | 3047 |
| C21 | rs2828515 | 24056599 | G | 0.124 | 28296 | 0.0093 | 1.13 | 0.138 | 2394 | 0.0024 | 1.14 | 0.139 | 3046 |
| C21 | rs2829040 | 24693776 | A | 0.452 | 28251 | 0.047 | 1.07 | 0.468 | 2392 | 0.23 | 1.04 | 0.460 | 3044 |
| C21 | rs2830357 | 26972748 | G | 0.575 | 28313 | 0.0019 | 1.11 | 0.600 | 2395 | 0.0025 | 1.10 | 0.597 | 3046 |
| C21 | rs2251540 | 26986173 | G | 0.651 | 28262 | 0.0027 | 1.11 | 0.674 | 2391 | 0.0089 | 1.09 | 0.669 | 3042 |
| C21 | rs1882757 | 40711890 | A | 0.507 | 28320 | 0.079 | 1.06 | 0.522 | 2393 | 0.068 | 1.06 | 0.521 | 3045 |
| C21 | rs3788014 | 42722104 | C | 0.925 | 28314 | 0.0012 | 1.24 | 0.938 | 2395 | 0.0019 | 1.20 | 0.937 | 3046 |
| C22 | rs2019061 | 17343340 | T | 0.689 | 28294 | 0.01 | 1.10 | 0.708 | 2388 | 0.0051 | 1.09 | 0.708 | 3038 |
| C22 | rs1978058 | 18288219 | T | 0.372 | 28137 | 0.000015 | 1.16 | 0.406 | 2388 | 0.00038 | 1.11 | 0.397 | 3034 |
| C22 | rs3804044 | 18432442 | T | 0.858 | 28284 | 0.0037 | 1.15 | 0.875 | 2395 | 0.0039 | 1.13 | 0.873 | 3046 |
| C22 | rs5992507 | 18437458 | A | 0.859 | 28309 | 0.004 | 1.15 | 0.875 | 2395 | 0.0042 | 1.13 | 0.873 | 3047 |
| C22 | rs2073778 | 18454575 | C | 0.858 | 28316 | 0.0036 | 1.15 | 0.875 | 2395 | 0.0046 | 1.13 | 0.873 | 3047 |
| C22 | rs2238798 | 18487729 | G | 0.859 | 28289 | 0.0045 | 1.15 | 0.875 | 2394 | 0.0057 | 1.13 | 0.873 | 3044 |
| C22 | rs178260 | 19654308 | T | 0.142 | 28310 | 0.029 | 1.10 | 0.154 | 2395 | 0.039 | 1.09 | 0.152 | 3047 |
| C22 | rs430305 | 19688522 | T | 0.829 | 28319 | 0.0096 | 1.12 | 0.844 | 2395 | 0.038 | 1.09 | 0.840 | 3047 |
| C22 | rs8135273 | 32527802 | C | 0.157 | 28046 | 0.025 | 1.10 | 0.170 | 2369 | 0.057 | 1.08 | 0.167 | 3012 |
| C22 | rs2267296 | 32549939 | G | 0.444 | 28313 | 0.0015 | 1.11 | 0.470 | 2394 | 0.00076 | 1.10 | 0.469 | 3046 |
| C22 | rs2071744 | 34147553 | C | 0.782 | 28278 | 0.012 | 1.11 | 0.798 | 2394 | 0.038 | 1.08 | 0.794 | 3044 |
| C22 | rs132717 | 34926598 | C | 0.618 | 28303 | 0.069 | 1.06 | 0.633 | 2395 | 0.56 | 1.02 | 0.622 | 3047 |
| C22 | rs7364143 | 34932129 | T | 0.279 | 28278 | 0.00042 | 1.13 | 0.305 | 2391 | 0.0024 | 1.10 | 0.299 | 3043 |
| C22 | rs5995259 | 34941809 | A | 0.338 | 28320 | 0.00058 | 1.12 | 0.365 | 2395 | 0.004 | 1.09 | 0.358 | 3047 |
| C22 | rs763086 | 34949003 | A | 0.301 | 28254 | 0.0059 | 1.10 | 0.322 | 2391 | 0.014 | 1.08 | 0.318 | 3038 |
| C22 | rs738407 | 42655288 | A | 0.640 | 28301 | 0.0014 | 1.12 | 0.664 | 2394 | 0.0018 | 1.10 | 0.662 | 3046 |
| C22 | rs739164 | 45063222 | A | 0.892 | 27998 | 0.0056 | 1.17 | 0.906 | 2330 | 0.018 | 1.12 | 0.903 | 2978 |
| CX | rs6638525 | 5464517 | C | 0.110 | 22852 | 0.016 | 1.16 | 0.126 | 1545 | 0.017 | 1.14 | 0.124 | 1962 |
| CX | rs1919026 | 5471853 | C | 0.195 | 22929 | 0.04 | 1.11 | 0.212 | 1550 | 0.22 | 1.06 | 0.204 | 1967 |
| CX | rs7892324 | 6679297 | C | 0.167 | 22905 | 0.0011 | 1.19 | 0.192 | 1542 | 0.005 | 1.14 | 0.186 | 1956 |
| CX | rs3013140 | 41283447 | G | 0.933 | 22939 | 0.042 | 1.19 | 0.943 | 1551 | 0.037 | 1.17 | 0.943 | 1968 |
| CX | rs5914778 | 56774956 | G | 0.804 | 22928 | 0.002 | 1.18 | 0.828 | 1550 | 0.0032 | 1.15 | 0.825 | 1966 |
| CX | rs2031751 | 67165559 | C | 0.941 | 22937 | 0.08 | 1.17 | 0.949 | 1550 | 0.051 | 1.17 | 0.949 | 1967 |
| CX | rs1576059 | 67171306 | T | 0.941 | 22937 | 0.076 | 1.17 | 0.949 | 1551 | 0.056 | 1.17 | 0.949 | 1968 |
| CX | rs7050085 | 67278825 | G | 0.908 | 22941 | 0.015 | 1.19 | 0.922 | 1550 | 0.0059 | 1.20 | 0.922 | 1967 |
| CX | rs2341629 | 70367614 | C | 0.470 | 22923 | 0.093 | 1.07 | 0.487 | 1550 | 0.16 | 1.05 | 0.483 | 1967 |
| CX | rs2213468 | 86099449 | G | 0.668 | 22877 | 0.047 | 1.09 | 0.687 | 1541 | 0.15 | 1.06 | 0.680 | 1958 |
| CX | rs6615536 | 92662125 | G | 0.988 | 22935 | 0.0094 | 1.77 | 0.993 | 1551 | 0.00098 | 1.97 | 0.994 | 1968 |
| CX | rs2227165 | 92880063 | C | 0.899 | 8732 | 0.00000074 | 1.56 | 0.933 | 1121 | 0.00013 | 1.37 | 0.924 | 1314 |
| CX | rs3788769 | 109423718 | C | 0.441 | 22401 | 0.0016 | 1.14 | 0.473 | 1534 | 0.0018 | 1.12 | 0.470 | 1944 |
| CX | rs2526763 | 116328390 | G | 0.365 | 22923 | 0.006 | 1.12 | 0.392 | 1549 | 0.0083 | 1.10 | 0.388 | 1965 |
| CX | rs1948488 | 139835516 | A | 0.222 | 22466 | 0.043 | 1.10 | 0.239 | 1527 | 0.02 | 1.11 | 0.240 | 1937 |
| CX | rs11094472 | 144273211 | G | 0.787 | 22928 | 0.00017 | 1.21 | 0.818 | 1545 | 0.004 | 1.14 | 0.808 | 1962 |

TABLE 4
Results of replication analysis in several populations. A total of 100 markers from
the genome-wide scan shown in Table 3 were selected for replication. The table shows results
of replication in cohorts from Atlanta and Durham (A - MI; B - CAD), Durham and Italy (C -
MI; D - CAD), John Hopkins, WTCCC and in combined replication samples (E - MI; F - CAD),
and results for the Icelandic discovery cohort and combined analysis for all the cohorts (G -
MI; H - CAD). The number of controls/cases is shown for each analysis.

| Table 4A - MI | | | | Atlanta (1273/646) | | | | | Philadelphia (497/570) | | | | |
|---|---|---|---|---|---|---|---|---|---|---|---|---|---|
| Chr | SNP | Pos_B36 | Allele | nc/na | Ctr. fr | Aff. fr | OR | P | nc/na | Ctr. fr | Aff. fr | OR | P |
| C06 | rs11751605 | 160883220 | C | 1273/643 | 0.155 | 0.163 | 1.06 | 0.53 | 497/566 | 0.131 | 0.135 | 1.04 | 0.77 |
| C10 | rs2243547 | 91000459 | G | | | | | | | | | | |
| C10 | rs1412444 | 90992907 | A | 581/367 | 0.333 | 0.356 | 1.10 | 0.31 | 496/570 | 0.326 | 0.353 | 1.13 | 0.19 |
| C01 | rs12134779 | 63168930 | T | 1260/578 | 0.285 | 0.289 | 1.02 | 0.78 | 497/567 | 0.270 | 0.271 | 1.01 | 0.95 |
| C11 | rs2163612 | 82856844 | G | 513/367 | 0.496 | 0.499 | 1.01 | 0.92 | 465/522 | 0.488 | 0.513 | 1.11 | 0.26 |
| C05 | rs254850 | 158599309 | T | | | | | | | | | | |
| C17 | rs9902941 | 17674485 | C | 1210/591 | 0.352 | 0.372 | 1.09 | 0.25 | 474/542 | 0.393 | 0.423 | 1.13 | 0.17 |
| C20 | rs6076623 | 4084671 | T | 1246/621 | 0.303 | 0.335 | 1.16 | 0.051 | 494/562 | 0.283 | 0.276 | 0.96 | 0.7 |
| C05 | rs4921437 | 158623529 | T | | | | | | | | | | |
| C05 | rs832540 | 56234959 | C | | | | | | | | | | |
| C19 | rs12459084 | 17031885 | T | | | | | | | | | | |
| C07 | rs1029396 | 116812056 | G | 572/363 | 0.082 | 0.094 | 1.15 | 0.39 | 491/562 | 0.074 | 0.070 | 0.94 | 0.72 |
| C17 | rs6502622 | 17775416 | G | 1227/593 | 0.366 | 0.401 | 1.16 | 0.042 | 475/540 | 0.409 | 0.433 | 1.10 | 0.28 |
| C17 | rs854787 | 17975744 | C | 507/360 | 0.399 | 0.475 | 1.36 | 0.0017 | 475/542 | 0.433 | 0.458 | 1.11 | 0.24 |
| C14 | rs7158073 | 99211442 | T | 575/362 | 0.408 | 0.419 | 1.04 | 0.65 | 480/549 | 0.426 | 0.448 | 1.09 | 0.31 |
| C10 | rs2243548 | 91000293 | A | | | | | | | | | | |
| C17 | rs4925119 | 17683629 | A | 1207/646 | 0.349 | 0.372 | 1.11 | 0.16 | 470/537 | 0.397 | 0.419 | 1.10 | 0.31 |
| C17 | rs3183702 | 17688014 | T | 539/362 | 0.337 | 0.391 | 1.26 | 0.019 | 469/531 | 0.393 | 0.427 | 1.15 | 0.13 |
| C17 | rs854813 | 17944570 | C | 539/364 | 0.388 | 0.449 | 1.29 | 0.0094 | 462/520 | 0.429 | 0.468 | 1.17 | 0.077 |
| C05 | rs953861 | 158705160 | G | | | | | | | | | | |
| C07 | rs12534186 | 117079572 | T | | | | | | | | | | |
| C12 | rs2417821 | 20081535 | T | | | | | | | | | | |
| C20 | rs1741318 | 4105600 | A | | | | | | | | | | |
| C06 | rs2074464 | 29516507 | T | 576/365 | 0.442 | 0.484 | 1.18 | 0.077 | 491/554 | 0.486 | 0.464 | 0.92 | 0.32 |
| C07 | rs324594 | 136305863 | C | | | | | | | | | | |
| C05 | rs270654 | 158497687 | G | | | | | | | | | | |
| C15 | rs4779984 | 30302218 | G | | | | | | | | | | |
| C05 | rs7709212 | 158696755 | T | | | | | | | | | | |
| C05 | rs10045431 | 158747111 | A | | | | | | | | | | |
| C17 | rs2955355 | 17889200 | C | 534/360 | 0.291 | 0.360 | 1.37 | 0.0024 | 476/544 | 0.352 | 0.362 | 1.05 | 0.63 |
| C11 | rs7944761 | 1361414 | T | | | | | | | | | | |
| C05 | rs4704400 | 76586766 | T | | | | | | 416/485 | 0.460 | 0.468 | 1.03 | 0.74 |
| C17 | rs4925114 | 17651995 | A | 521/361 | 0.343 | 0.378 | 1.17 | 0.13 | 473/539 | 0.403 | 0.400 | 0.99 | 0.89 |
| C05 | rs1433048 | 158688423 | G | | | | | | | | | | |
| C05 | rs7447732 | 158461713 | C | 1225/544 | 0.315 | 0.312 | 0.99 | 0.85 | 497/567 | 0.272 | 0.311 | 1.21 | 0.045 |
| C02 | rs2244871 | 128115310 | C | 500/349 | 0.431 | 0.430 | 1.00 | 0.96 | 456/509 | 0.459 | 0.449 | 0.96 | 0.64 |
| C01 | rs2946534 | 18647370 | T | | | | | | | | | | |
| C04 | rs7661204 | 45728C07 | T | | | | | | | | | | |
| C05 | rs6556861 | 94554256 | G | | | | | | | | | | |
| C06 | rs7753765 | 71732495 | A | 532/366 | 0.172 | 0.182 | 1.07 | 0.6 | 479/533 | 0.175 | 0.174 | 0.99 | 0.96 |
| C05 | rs870347 | 6898035 | T | | | | | | | | | | |
| C10 | rs12269612 | 15475877 | T | | | | | | | | | | |
| C15 | rs2110209 | 24946433 | A | | | | | | | | | | |
| C02 | rs12329252 | 212093968 | T | 1250/554 | 0.681 | 0.680 | 0.99 | 0.94 | 492/569 | 0.682 | 0.683 | 1.00 | 0.97 |
| C14 | rs8003722 | 68388015 | A | 1029/472 | 0.147 | 0.151 | 1.04 | 0.73 | | | | | |
| C21 | rs2828495 | 24038081 | A | | | | | | | | | | |
| C05 | rs270661 | 158492732 | A | | | | | | | | | | |
| C08 | rs334198 | 18933089 | C | 542/367 | 0.918 | 0.914 | 0.95 | 0.78 | 475/543 | 0.900 | 0.912 | 1.15 | 0.37 |
| C01 | rs2476601 | 114179091 | A | 1188/280 | 0.093 | 0.113 | 1.23 | 0.18 | 462/540 | 0.093 | 0.096 | 1.04 | 0.81 |
| C13 | rs4769613 | 28036609 | T | | | | | | | | | | |
| C08 | rs3102526 | 96858538 | G | | | | | | | | | | |
| C08 | rs3134517 | 96859237 | G | | | | | | | | | | |
| C01 | rs3748744 | 19828833 | G | | | | | | | | | | |
| C04 | rs4282162 | 21426819 | T | | | | | | | | | | |
| C16 | rs8050136 | 52373776 | A | | | | | | 488/561 | 0.408 | 0.415 | 1.03 | 0.73 |
| C16 | rs3751812 | 52375961 | T | | | | | | 490/567 | 0.397 | 0.405 | 1.03 | 0.71 |
| C17 | rs854793 | 17971249 | A | 539/369 | 0.213 | 0.255 | 1.26 | 0.04 | 454/505 | 0.246 | 0.260 | 1.08 | 0.46 |
| C10 | rs4880711 | 5217871 | C | | | | | | | | | | |
| C12 | rs12304836 | 124654446 | C | 1217/588 | 0.152 | 0.151 | 0.99 | 0.88 | 472/532 | 0.145 | 0.173 | 1.23 | 0.089 |
| C05 | rs38055 | 52596401 | T | 1259/556 | 0.311 | 0.341 | 1.15 | 0.072 | 491/565 | 0.305 | 0.340 | 1.17 | 0.092 |
| C09 | rs2297538 | 138684489 | G | | | | | | 404/454 | 0.786 | 0.739 | 0.77 | 0.023 |
| C16 | rs1870843 | 81316815 | T | | | | | | | | | | |
| C06 | rs6905466 | 19958854 | T | | | | | | | | | | |
| C12 | rs11045689 | 21092930 | G | 1196/419 | 0.345 | 0.344 | 0.99 | 0.95 | 492/556 | 0.362 | 0.346 | 0.93 | 0.46 |
| C13 | rs1887263 | 22381716 | A | | | | | | | | | | |
| C18 | rs4271662 | 26051291 | C | 1216/551 | 0.598 | 0.613 | 1.07 | 0.38 | 487/553 | 0.611 | 0.613 | 1.01 | 0.92 |
| C18 | rs1365254 | 70238283 | T | | | | | | | | | | |
| C06 | rs3016536 | 162157939 | G | | | | | | | | | | |
| C12 | rs3742049 | 119438873 | C | 584/364 | 0.901 | 0.905 | 1.05 | 0.75 | | | | | |
| C03 | rs9874646 | 62437858 | T | 1193/414 | 0.684 | 0.687 | 1.01 | 0.86 | 492/559 | 0.648 | 0.678 | 1.14 | 0.15 |
| C14 | rs2281519 | 93846385 | T | | | | | | | | | | |
| C02 | rs6747236 | 35901725 | T | 975/276 | 0.756 | 0.777 | 1.12 | 0.31 | 460/523 | 0.771 | 0.750 | 0.90 | 0.3 |
| C04 | rs6534683 | 129601758 | T | | | | | | | | | | |
| C01 | rs2991515 | 57656898 | T | 1136/387 | 0.453 | 0.463 | 1.04 | 0.66 | 427/497 | 0.474 | 0.466 | 0.97 | 0.72 |
| C04 | rs4697653 | 10337857 | G | | | | | | 447/520 | 0.534 | 0.533 | 1.00 | 0.97 |
| C06 | rs9380681 | 37638433 | C | | | | | | | | | | |
| C10 | rs11199158 | 85297471 | G | | | | | | | | | | |
| C13 | rs1375719 | 102208783 | C | 584/362 | 0.529 | 0.514 | 0.94 | 0.52 | | | | | |
| C08 | rs3931282 | 129179963 | T | 587/362 | 0.532 | 0.562 | 1.13 | 0.21 | | | | | |
| C14 | rs17776453 | 100774793 | G | | | | | | | | | | |
| C01 | rs12724604 | 20948045 | A | | | | | | | | | | |
| C05 | rs950195 | 97466690 | G | | | | | | | | | | |
| C05 | rs728676 | 118087433 | T | 1012/464 | 0.465 | 0.498 | 1.14 | 0.1 | | | | | |
| C06 | rs7739802 | 162154323 | T | | | | | | | | | | |
| C10 | rs7086224 | 30857283 | A | | | | | | | | | | |
| C01 | rs4845552 | 151746622 | G | | | | | | | | | | |
| C08 | rs10504537 | 73402234 | A | | | | | | | | | | |
| C02 | rs1395930 | 123468570 | T | 1216/559 | 0.531 | 0.484 | 0.83 | 0.0087 | 489/555 | 0.516 | 0.529 | 1.05 | 0.57 |
| C02 | rs3943703 | 127613790 | C | | | | | | | | | | |
| C10 | rs2279434 | 45275070 | T | 513/362 | 0.084 | 0.070 | 0.83 | 0.3 | 472/532 | 0.097 | 0.081 | 0.81 | 0.19 |
| C10 | rs11198993 | 85244450 | A | | | | | | | | | | |
| C10 | rs7921473 | 85156395 | G | | | | | | | | | | |
| C12 | rs1465026 | 65789635 | G | | | | | | | | | | |
| C12 | rs1568383 | 88017285 | T | 537/370 | 0.127 | 0.105 | 0.81 | 0.17 | 467/528 | 0.123 | 0.116 | 0.94 | 0.65 |
| C08 | rs4732849 | 28249765 | T | 1160/411 | 0.316 | 0.330 | 1.06 | 0.48 | 486/554 | 0.315 | 0.312 | 0.99 | 0.9 |
| C08 | rs7006687 | 18277862 | T | | | | | | | | | | |
| C02 | rs921083 | 176453128 | G | | | | | | 493/563 | 0.813 | 0.804 | 0.94 | 0.57 |
| C02 | rs6709904 | 43933828 | A | | | | | | | | | | |
| C14 | rs1957860 | 53499105 | T | 1188/419 | 0.476 | 0.486 | 1.04 | 0.62 | 492/565 | 0.497 | 0.471 | 0.90 | 0.23 |
| C14 | rs4444235 | 53480669 | T | 1160/404 | 0.534 | 0.524 | 0.96 | 0.62 | 489/551 | 0.535 | 0.521 | 0.95 | 0.53 |
| Table 4B - CAD | | | | | | | | | | | | | |
| | | Pos Build | | Atlanta (1273/772) | | | | | Philadelphia (497/700) | | | | |
| Chr | SNP | 36 | Allele | nc/na | Ctr. fr | Aff. fr | OR | P | nc/na | Ctr. fr | Aff. fr | OR | P |
| C06 | rs11751605 | 160883220 | C | 1273/772 | 0.155 | 0.163 | 1.07 | 0.47 | 497/693 | 0.131 | 0.139 | 1.08 | 0.55 |
| C20 | rs6076623 | 4084671 | T | 1246/748 | 0.303 | 0.326 | 1.11 | 0.14 | 494/689 | 0.283 | 0.275 | 0.96 | 0.65 |
| C10 | rs1412444 | 90992907 | A | 581/475 | 0.333 | 0.348 | 1.07 | 0.46 | 496/700 | 0.326 | 0.353 | 1.13 | 0.17 |
| C11 | rs2163612 | 82856844 | G | 513/466 | 0.496 | 0.510 | 1.06 | 0.55 | 465/539 | 0.488 | 0.512 | 1.10 | 0.29 |
| C07 | rs1029396 | 116812056 | G | 572/471 | 0.082 | 0.086 | 1.05 | 0.75 | 491/692 | 0.074 | 0.072 | 0.97 | 0.85 |
| C10 | rs2243547 | 91000459 | G | | | | | | | | | | |
| C07 | rs12534186 | 117079572 | T | | | | | | | | | | |
| C01 | rs12134779 | 63168930 | T | 1260/706 | 0.285 | 0.290 | 1.03 | 0.73 | 497/695 | 0.270 | 0.260 | 0.95 | 0.59 |
| C14 | rs7158073 | 99211442 | T | 575/469 | 0.408 | 0.414 | 1.02 | 0.79 | 480/673 | 0.426 | 0.442 | 1.07 | 0.44 |
| C05 | rs254850 | 158599309 | T | | | | | | | | | | |
| C12 | rs2417821 | 20081535 | T | | | | | | | | | | |
| C04 | rs7661204 | 45728007 | T | | | | | | | | | | |
| C05 | rs4921437 | 158623529 | T | | | | | | | | | | |
| C05 | rs832540 | 56234959 | C | | | | | | | | | | |
| C07 | rs324594 | 136305863 | C | | | | | | | | | | |
| C20 | rs1741318 | 4105600 | A | | | | | | | | | | |
| C17 | rs9902941 | 17674485 | C | 1210/719 | 0.352 | 0.373 | 1.09 | 0.21 | 474/557 | 0.393 | 0.419 | 1.11 | 0.24 |
| C05 | rs7709212 | 158696755 | T | | | | | | | | | | |
| C01 | rs2946534 | 18647370 | T | | | | | | | | | | |
| C05 | rs6556861 | 94554256 | G | | | | | | | | | | |
| C16 | rs8050136 | 52373776 | A | | | | | | 488/685 | 0.408 | 0.420 | 1.05 | 0.54 |
| C16 | rs3751812 | 52375961 | T | | | | | | 490/692 | 0.397 | 0.410 | 1.05 | 0.53 |
| C13 | rs4769613 | 28036609 | T | | | | | | | | | | |
| C10 | rs2243548 | 91000293 | A | | | | | | | | | | |
| C19 | rs12459084 | 17031885 | T | | | | | | | | | | |
| C06 | rs2074464 | 29516507 | T | 576/474 | 0.442 | 0.465 | 1.10 | 0.28 | 491/677 | 0.486 | 0.470 | 0.94 | 0.44 |
| C02 | rs2244871 | 128115310 | C | 500/444 | 0.431 | 0.422 | 0.96 | 0.7 | 456/525 | 0.459 | 0.451 | 0.97 | 0.72 |
| C05 | rs270654 | 158497687 | G | | | | | | | | | | |
| C17 | rs854787 | 17975744 | C | 507/459 | 0.399 | 0.459 | 1.27 | 0.0086 | 475/560 | 0.433 | 0.454 | 1.09 | 0.34 |
| C11 | rs7944761 | 1361414 | T | | | | | | | | | | |
| C15 | rs4779984 | 30302218 | G | | | | | | | | | | |
| C17 | rs6502622 | 17775416 | G | 1227/719 | 0.366 | 0.403 | 1.17 | 0.021 | 475/558 | 0.409 | 0.429 | 1.08 | 0.36 |
| C17 | rs3183702 | 17688014 | T | 539/465 | 0.337 | 0.387 | 1.24 | 0.019 | 469/548 | 0.393 | 0.422 | 1.12 | 0.2 |
| C05 | rs1433048 | 158688423 | G | | | | | | | | | | |
| C17 | rs4925119 | 17683629 | A | 1207/770 | 0.349 | 0.373 | 1.11 | 0.13 | 470/555 | 0.397 | 0.414 | 1.08 | 0.42 |
| C01 | rs2476601 | 114179091 | A | 1188/323 | 0.093 | 0.121 | 1.33 | 0.044 | 462/666 | 0.093 | 0.098 | 1.05 | 0.72 |
| C05 | rs870347 | 6898035 | T | | | | | | | | | | |
| C08 | rs334198 | 18933089 | C | 542/472 | 0.918 | 0.908 | 0.88 | 0.42 | 475/561 | 0.900 | 0.912 | 1.15 | 0.36 |
| C17 | rs854813 | 17944570 | C | 539/467 | 0.388 | 0.442 | 1.25 | 0.013 | 462/538 | 0.429 | 0.465 | 1.16 | 0.11 |
| C06 | rs7753765 | 71732495 | A | 532/468 | 0.172 | 0.174 | 1.02 | 0.9 | 479/551 | 0.175 | 0.172 | 0.97 | 0.82 |
| C17 | rs4925114 | 17651995 | A | 521/461 | 0.343 | 0.379 | 1.17 | 0.098 | 473/555 | 0.403 | 0.395 | 0.97 | 0.74 |
| C05 | rs270661 | 158492732 | A | | | | | | | | | | |
| C05 | rs953861 | 158705160 | G | | | | | | | | | | |
| C05 | rs10045431 | 158747111 | A | | | | | | | | | | |
| C14 | rs8003722 | 68388015 | A | 1029/530 | 0.147 | 0.151 | 1.03 | 0.75 | | | | | |
| C09 | rs2297538 | 138684489 | G | | | | | | 404/555 | 0.786 | 0.749 | 0.81 | 0.057 |
| C02 | rs12329252 | 212093968 | T | 1250/681 | 0.681 | 0.675 | 0.97 | 0.7 | 492/696 | 0.682 | 0.685 | 1.02 | 0.86 |
| C01 | rs3748744 | 19828833 | G | | | | | | | | | | |
| C05 | rs4704400 | 76586766 | T | | | | | | 416/576 | 0.460 | 0.462 | 1.01 | 0.95 |
| C08 | rs3102526 | 96858538 | G | | | | | | | | | | |
| C15 | rs2110209 | 24946433 | A | | | | | | | | | | |
| C16 | rs1870843 | 81316815 | T | | | | | | | | | | |
| C08 | rs3134517 | 96859237 | G | | | | | | | | | | |
| C21 | rs2828495 | 24038081 | A | | | | | | | | | | |
| C04 | rs4282162 | 21426819 | T | | | | | | | | | | |
| C17 | rs2955355 | 17889200 | C | 534/463 | 0.291 | 0.351 | 1.32 | 0.0043 | 476/562 | 0.352 | 0.359 | 1.03 | 0.72 |
| C12 | rs11045689 | 21092930 | G | 1196/462 | 0.345 | 0.345 | 1.00 | 0.99 | 492/680 | 0.362 | 0.347 | 0.94 | 0.46 |
| C05 | rs7447732 | 158461713 | C | 1225/669 | 0.315 | 0.299 | 0.93 | 0.32 | 497/691 | 0.272 | 0.310 | 1.20 | 0.044 |
| C18 | rs1365254 | 70238283 | T | | | | | | | | | | |
| C13 | rs1887263 | 22381716 | A | | | | | | | | | | |
| C12 | rs12304836 | 124654446 | C | 1217/712 | 0.152 | 0.151 | 0.99 | 0.9 | 472/550 | 0.145 | 0.170 | 1.21 | 0.12 |
| C05 | rs38055 | 52596401 | T | 1259/680 | 0.311 | 0.337 | 1.13 | 0.096 | 491/690 | 0.305 | 0.341 | 1.17 | 0.073 |
| C04 | rs4697653 | 10337857 | G | | | | | | 447/619 | 0.534 | 0.537 | 1.01 | 0.87 |
| C05 | rs728676 | 118087433 | T | 1012/522 | 0.465 | 0.496 | 1.13 | 0.11 | | | | | |
| C06 | rs6905466 | 19958854 | T | | | | | | | | | | |
| C10 | rs11199158 | 85297471 | G | | | | | | | | | | |
| C01 | rs4845552 | 151746622 | G | | | | | | | | | | |
| C02 | rs1395930 | 123468570 | T | 1216/686 | 0.531 | 0.483 | 0.83 | 0.0044 | 489/681 | 0.516 | 0.524 | 1.03 | 0.71 |
| C04 | rs6534683 | 129601758 | T | | | | | | | | | | |
| C02 | rs6747236 | 35901725 | T | 975/312 | 0.756 | 0.782 | 1.16 | 0.19 | 460/640 | 0.771 | 0.761 | 0.95 | 0.6 |
| C06 | rs3016536 | 162157939 | G | | | | | | | | | | |
| C10 | rs12269612 | 15475877 | T | | | | | | | | | | |
| C17 | rs854793 | 17971249 | A | 539/475 | 0.213 | 0.252 | 1.24 | 0.042 | 454/522 | 0.246 | 0.259 | 1.07 | 0.51 |
| C12 | rs3742049 | 119438873 | C | 584/470 | 0.901 | 0.904 | 1.04 | 0.78 | | | | | |
| C01 | rs2991515 | 57656898 | T | 1136/430 | 0.453 | 0.462 | 1.03 | 0.68 | 427/621 | 0.474 | 0.461 | 0.95 | 0.56 |
| C18 | rs4271662 | 26051291 | C | 1216/676 | 0.598 | 0.611 | 1.06 | 0.43 | 487/676 | 0.611 | 0.620 | 1.04 | 0.66 |
| C06 | rs7739802 | 162154323 | T | | | | | | | | | | |
| C08 | rs10504537 | 73402234 | A | | | | | | | | | | |
| C14 | rs17776453 | 100774793 | G | | | | | | | | | | |
| C10 | rs7086224 | 30857283 | A | | | | | | | | | | |
| C01 | rs12724604 | 20948045 | A | | | | | | | | | | |
| C03 | rs9874646 | 62437858 | T | 1193/457 | 0.684 | 0.690 | 1.03 | 0.72 | 492/683 | 0.648 | 0.679 | 1.15 | 0.13 |
| C05 | rs950195 | 97466690 | G | | | | | | | | | | |
| C14 | rs2281519 | 93846385 | T | | | | | | | | | | |
| C10 | rs4880711 | 5217871 | C | | | | | | | | | | |
| C08 | rs3931282 | 129179963 | T | 587/469 | 0.532 | 0.560 | 1.12 | 0.21 | | | | | |
| C10 | rs11198993 | 85244450 | A | | | | | | | | | | |
| C12 | rs1568383 | 88017285 | T | 537/472 | 0.127 | 0.105 | 0.81 | 0.13 | 467/546 | 0.123 | 0.116 | 0.94 | 0.64 |
| C06 | rs9380681 | 37638433 | C | | | | | | | | | | |
| C02 | rs6709904 | 43933828 | A | 959/51 | 0.903 | 0.912 | 1.12 | 0.76 | | | | | |
| C10 | rs2279434 | 45275070 | T | 513/462 | 0.084 | 0.076 | 0.90 | 0.51 | 472/550 | 0.097 | 0.078 | 0.79 | 0.12 |
| C13 | rs1375719 | 102208783 | C | 584/469 | 0.529 | 0.499 | 0.89 | 0.17 | | | | | |
| C10 | rs7921473 | 85156395 | G | | | | | | | | | | |
| C14 | rs1957860 | 53499105 | T | 1188/462 | 0.476 | 0.483 | 1.03 | 0.71 | 492/691 | 0.497 | 0.475 | 0.91 | 0.29 |
| C02 | rs3943703 | 127613790 | C | | | | | | | | | | |
| C08 | rs7006687 | 18277862 | T | | | | | | | | | | |
| C12 | rs1465026 | 65789635 | G | | | | | | | | | | |
| C14 | rs4444235 | 53480669 | T | 1160/445 | 0.534 | 0.527 | 0.97 | 0.74 | 489/673 | 0.535 | 0.516 | 0.93 | 0.36 |
| C02 | rs921083 | 176453128 | G | | | | | | 493/689 | 0.813 | 0.810 | 0.98 | 0.83 |
| C08 | rs4732849 | 28249765 | T | 1160/454 | 0.316 | 0.326 | 1.05 | 0.6 | 486/678 | 0.315 | 0.316 | 1.00 | 0.97 |
| Table 4C - MI | | | | Durham (737/1199) | | | | | Italy (1076/657) | | | | |
| Chr | SNP | Pos_B36 | Allele | nc/na | Ctr. fr | Aff. fr | OR | P | nc/na | Ctr. fr | Aff. fr | OR | P |
| C06 | rs11751605 | 160883220 | C | 731/1187 | 0.158 | 0.174 | 1.12 | 0.21 | 1075/656 | 0.083 | 0.100 | 1.22 | 0.099 |
| C10 | rs2243547 | 91000459 | G | | | | | | | | | | |
| C10 | rs1412444 | 90992907 | A | 730/1190 | 0.344 | 0.356 | 1.05 | 0.45 | 1068/646 | 0.309 | 0.328 | 1.09 | 0.25 |
| C01 | rs12134779 | 63168930 | T | 731/1191 | 0.282 | 0.309 | 1.14 | 0.078 | 1055/646 | 0.248 | 0.266 | 1.10 | 0.25 |
| C11 | rs2163612 | 62856844 | G | 732/1182 | 0.484 | 0.491 | 1.03 | 0.67 | | | | | |
| C05 | rs254850 | 158599309 | T | | | | | | | | | | |
| C17 | rs9902941 | 17674485 | C | 736/1150 | 0.355 | 0.378 | 1.10 | 0.15 | | | | | |
| C20 | rs6076623 | 4084671 | T | 737/1195 | 0.307 | 0.323 | 1.08 | 0.28 | 1062/649 | 0.222 | 0.213 | 0.95 | 0.57 |
| C05 | rs4921437 | 158623529 | T | 725/1194 | 0.197 | 0.194 | 0.98 | 0.8 | 1045/639 | 0.171 | 0.174 | 1.02 | 0.81 |
| C05 | rs832540 | 56234959 | C | 711/1175 | 0.370 | 0.385 | 1.06 | 0.36 | 1052/642 | 0.372 | 0.363 | 0.96 | 0.59 |
| C19 | rs12459084 | 17031885 | T | 732/1196 | 0.413 | 0.409 | 0.98 | 0.82 | | | | | |
| C07 | rs1029396 | 116812056 | G | 735/1197 | 0.083 | 0.102 | 1.26 | 0.045 | 1076/657 | 0.062 | 0.053 | 0.84 | 0.25 |
| C17 | rs6502622 | 17775416 | G | 734/1150 | 0.377 | 0.400 | 1.10 | 0.17 | 1048/639 | 0.456 | 0.450 | 0.98 | 0.75 |
| C17 | rs854787 | 17975744 | C | 732/1180 | 0.413 | 0.438 | 1.11 | 0.13 | 1017/611 | 0.501 | 0.489 | 0.95 | 0.52 |
| C14 | rs7158073 | 99211442 | T | 719/1159 | 0.429 | 0.461 | 1.14 | 0.058 | 490/270 | 0.458 | 0.407 | 0.81 | 0.056 |
| C10 | rs2243548 | 91000293 | A | | | | | | | | | | |
| C17 | rs4925119 | 17683629 | A | 729/1173 | 0.350 | 0.374 | 1.11 | 0.13 | 1026/640 | 0.437 | 0.430 | 0.97 | 0.67 |
| C17 | rs3183702 | 17688014 | T | 572/926 | 0.355 | 0.369 | 1.06 | 0.44 | 1059/641 | 0.435 | 0.422 | 0.95 | 0.46 |
| C17 | rs854813 | 17944570 | C | 209/412 | 0.392 | 0.417 | 1.11 | 0.39 | 1057/646 | 0.502 | 0.469 | 0.88 | 0.059 |
| C05 | rs953861 | 158705160 | G | | | | | | | | | | |
| C07 | rs12534186 | 117079572 | T | | | | | | | | | | |
| C12 | rs2417821 | 20081535 | T | 725/1186 | 0.720 | 0.721 | 1.01 | 0.93 | 1042/641 | 0.668 | 0.670 | 1.01 | 0.92 |
| C20 | rs1741318 | 4105600 | A | | | | | | | | | | |
| C06 | rs2074464 | 29516507 | T | 728/1184 | 0.451 | 0.466 | 1.06 | 0.37 | 1034/634 | 0.462 | 0.452 | 0.96 | 0.58 |
| C07 | rs324594 | 136305863 | C | | | | | | | | | | |
| C05 | rs270654 | 158497687 | G | | | | | | | | | | |
| C15 | rs4779984 | 30302218 | G | | | | | | | | | | |
| C05 | rs7709212 | 158696755 | T | | | | | | | | | | |
| C05 | rs10045431 | 158747111 | A | | | | | | | | | | |
| C17 | rs2955355 | 17889200 | C | 212/413 | 0.302 | 0.337 | 1.17 | 0.21 | 1052/614 | 0.402 | 0.374 | 0.89 | 0.11 |
| C11 | rs7944761 | 1361414 | T | | | | | | | | | | |
| C05 | rs4704400 | 76586766 | T | | | | | | | | | | |
| C17 | rs4925114 | 17651995 | A | 211/413 | 0.353 | 0.389 | 1.16 | 0.22 | | | | | |
| C05 | rs1433048 | 158688423 | G | | | | | | | | | | |
| C05 | rs7447732 | 158461713 | C | 732/1198 | 0.328 | 0.314 | 0.94 | 0.37 | 1067/643 | 0.305 | 0.285 | 0.91 | 0.23 |
| C02 | rs2244871 | 128115310 | C | 563/924 | 0.388 | 0.439 | 1.24 | 0.0059 | | | | | |
| C01 | rs2946534 | 18647370 | T | | | | | | | | | | |
| C04 | rs7661204 | 45728007 | T | 720/1183 | 0.411 | 0.435 | 1.10 | 0.14 | | | | | |
| C05 | rs6556861 | 94554256 | G | 719/1169 | 0.631 | 0.615 | 0.93 | 0.33 | 1056/637 | 0.652 | 0.633 | 0.92 | 0.27 |
| C06 | rs7753765 | 71732495 | A | | | | | | | | | | |
| C05 | rs870347 | 6898035 | T | | | | | | | | | | |
| C10 | rs12269612 | 15475877 | T | 734/1196 | 0.382 | 0.361 | 0.91 | 0.19 | 1055/634 | 0.382 | 0.363 | 0.92 | 0.26 |
| C15 | rs2110209 | 24946433 | A | 737/1191 | 0.035 | 0.032 | 0.93 | 0.7 | | | | | |
| C02 | rs12329252 | 212093968 | T | 737/1197 | 0.659 | 0.665 | 1.03 | 0.69 | 1047/642 | 0.713 | 0.694 | 0.91 | 0.23 |
| C14 | rs8003722 | 68388015 | A | 735/1186 | 0.144 | 0.136 | 0.94 | 0.48 | | | | | |
| C21 | rs2828495 | 24038081 | A | | | | | | | | | | |
| C05 | rs270661 | 158492732 | A | 736/1196 | 0.209 | 0.189 | 0.89 | 0.15 | 1066/652 | 0.194 | 0.173 | 0.87 | 0.12 |
| C08 | rs334198 | 18933089 | C | | | | | | | | | | |
| C01 | rs2476601 | 114179091 | A | 572/921 | 0.087 | 0.097 | 1.14 | 0.33 | 1049/647 | 0.062 | 0.070 | 1.12 | 0.42 |
| C13 | rs4769613 | 28036609 | T | 733/1190 | 0.458 | 0.463 | 1.02 | 0.77 | 1052/632 | 0.498 | 0.487 | 0.96 | 0.53 |
| C08 | rs3102526 | 96858538 | G | | | | | | | | | | |
| C08 | rs3134517 | 96859237 | G | | | | | | | | | | |
| C01 | rs3748744 | 19828833 | G | | | | | | | | | | |
| C04 | rs4282162 | 21426819 | T | 732/1198 | 0.488 | 0.488 | 1.00 | 0.99 | | | | | |
| C16 | rs8050136 | 52373776 | A | | | | | | 1064/635 | 0.414 | 0.425 | 1.05 | 0.52 |
| C16 | rs3751812 | 52375961 | T | | | | | | 1070/651 | 0.412 | 0.420 | 1.04 | 0.63 |
| C17 | rs854793 | 17971249 | A | 213/393 | 0.202 | 0.224 | 1.14 | 0.37 | 1031/649 | 0.268 | 0.240 | 0.87 | 0.077 |
| C10 | rs4880711 | 5217871 | C | | | | | | | | | | |
| C12 | rs12304836 | 124654446 | C | 729/1172 | 0.147 | 0.145 | 0.99 | 0.91 | | | | | |
| C05 | rs380555 | 2596401 | T | 729/1194 | 0.306 | 0.311 | 1.02 | 0.75 | 1054/641 | 0.316 | 0.306 | 0.95 | 0.54 |
| C09 | rs2297538 | 138684489 | G | 736/1193 | 0.798 | 0.795 | 0.98 | 0.79 | 712/231 | 0.754 | 0.755 | 1.01 | 0.96 |
| C16 | rs18708438 | 1316815 | T | | | | | | | | | | |
| C06 | rs6905466 | 19958854 | T | | | | | | | | | | |
| C12 | rs11045689 | 21092930 | G | 734/1195 | 0.328 | 0.365 | 1.18 | 0.019 | | | | | |
| C13 | rs1887263 | 22381716 | A | | | | | | | | | | |
| C18 | rs4271662 | 26051291 | C | 736/1194 | 0.596 | 0.601 | 1.02 | 0.76 | 1045/621 | 0.644 | 0.634 | 0.96 | 0.55 |
| C18 | rs1365254 | 70238283 | T | | | | | | | | | | |
| C06 | rs3016536 | 162157939 | G | | | | | | | | | | |
| C12 | rs3742049 | 119438873 | C | 120/254 | 0.896 | 0.884 | 0.88 | 0.63 | | | | | |
| C03 | rs9874646 | 62437858 | T | 735/1199 | 0.686 | 0.681 | 0.98 | 0.74 | | | | | |
| C14 | rs2281519 | 93846385 | T | | | | | | | | | | |
| C02 | rs6747236 | 35901725 | T | 432/1102 | 0.760 | 0.774 | 1.08 | 0.44 | | | | | |
| C04 | rs6534683 | 129601758 | T | | | | | | | | | | |
| C01 | rs2991515 | 57656898 | T | | | | | | | | | | |
| C04 | rs4697653 | 10337857 | G | 723/1187 | 0.558 | 0.540 | 0.93 | 0.27 | 1019/624 | 0.546 | 0.559 | 1.06 | 0.44 |
| C06 | rs9380681 | 37638433 | C | | | | | | | | | | |
| C10 | rs11199158 | 85297471 | G | 734/1195 | 0.636 | 0.638 | 1.01 | 0.89 | 1049/641 | 0.653 | 0.651 | 0.99 | 0.91 |
| C13 | rs1375719 | 102208783 | C | 116/250 | 0.504 | 0.464 | 0.85 | 0.31 | | | | | |
| C08 | rs3931282 | 129179963 | T | 732/1182 | 0.524 | 0.542 | 1.08 | 0.27 | | | | | |
| C14 | rs17776453 | 100774793 | G | | | | | | | | | | |
| C01 | rs12724604 | 20948045 | A | | | | | | | | | | |
| C05 | rs950195 | 97466690 | G | 727/1193 | 0.646 | 0.633 | 0.94 | 0.39 | 1036/637 | 0.647 | 0.617 | 0.88 | 0.083 |
| C05 | rs728676 | 118087433 | T | 718/1152 | 0.493 | 0.486 | 0.97 | 0.66 | | | | | |
| C06 | rs7739802 | 162154323 | T | | | | | | | | | | |
| C10 | rs7086224 | 30857283 | A | | | | | | | | | | |
| C01 | rs4845552 | 151746622 | G | 736/1197 | 0.140 | 0.154 | 1.12 | 0.24 | 1060/647 | 0.153 | 0.123 | 0.78 | 0.014 |
| C08 | rs10504537 | 73402234 | A | | | | | | | | | | |
| C02 | rs1395930 | 123468570 | T | 737/1188 | 0.507 | 0.508 | 1.00 | 0.97 | 1037/638 | 0.548 | 0.571 | 1.10 | 0.18 |
| C02 | rs3943703 | 127613790 | C | | | | | | | | | | |
| C10 | rs2279434 | 45275070 | T | | | | | | | | | | |
| C10 | rs11198993 | 85244450 | A | 732/1192 | 0.774 | 0.759 | 0.92 | 0.3 | 1048/641 | 0.776 | 0.786 | 1.06 | 0.49 |
| C10 | rs7921473 | 85156395 | G | 733/1177 | 0.840 | 0.828 | 0.91 | 0.3 | 1064/644 | 0.823 | 0.816 | 0.95 | 0.61 |
| C12 | rs1465026 | 65789635 | G | 731/1189 | 0.837 | 0.821 | 0.90 | 0.21 | 1061/654 | 0.842 | 0.847 | 1.04 | 0.67 |
| C12 | rs1568383 | 88017285 | T | | | | | | | | | | |
| C08 | rs4732849 | 28249765 | T | 724/1188 | 0.330 | 0.314 | 0.93 | 0.3 | | | | | |
| C08 | rs7006687 | 18277862 | T | 730/1196 | 0.567 | 0.549 | 0.93 | 0.27 | 1056/641 | 0.561 | 0.512 | 0.82 | 0.0059 |
| C02 | rs921083 | 176453128 | G | 734/1199 | 0.809 | 0.817 | 1.06 | 0.5 | 1057/636 | 0.828 | 0.831 | 1.02 | 0.81 |
| C02 | rs6709904 | 43933828 | A | 682/1110 | 0.891 | 0.885 | 0.94 | 0.58 | | | | | |
| C14 | rs1957860 | 53499105 | T | 737/1198 | 0.455 | 0.483 | 1.12 | 0.082 | | | | | |
| C14 | rs4444235 | 53480669 | T | 733/1186 | 0.548 | 0.527 | 0.92 | 0.22 | | | | | |
| Table 4D - CAD | | | | | | | | | | | | | |
| | | Pos Build | | Durham (737/1199) | | | | | Italy (1076/1122) | | | | |
| Chr | SNP | 36 | Allele | nc/na | Ctr. fr | Aff. fr | OR | P | nc/na | Ctr. fr | Aff. fr | OR | P |
| C06 | rs11751605 | 160883220 | C | 731/1187 | 0.158 | 0.174 | 1.12 | 0.21 | 1075/1122 | 0.083 | 0.099 | 1.21 | 0.071 |
| C20 | rs6076623 | 4084671 | T | 737/1195 | 0.307 | 0.323 | 1.08 | 0.28 | 1062/1105 | 0.222 | 0.222 | 1.00 | 1 |
| C10 | rs1412444 | 90992907 | A | 730/1190 | 0.344 | 0.356 | 1.05 | 0.45 | 1068/1105 | 0.309 | 0.330 | 1.10 | 0.15 |
| C11 | rs2163612 | 82856844 | G | 732/1182 | 0.484 | 0.491 | 1.03 | 0.67 | | | | | |
| C07 | rs1029396 | 116812056 | G | 735/1197 | 0.083 | 0.102 | 1.26 | 0.045 | 1076/1121 | 0.062 | 0.052 | 0.83 | 0.15 |
| C10 | rs2243547 | 91000459 | G | | | | | | | | | | |
| C07 | rs12534186 | 117079572 | T | | | | | | | | | | |
| C01 | rs12134779 | 63168930 | T | 731/1191 | 0.282 | 0.309 | 1.14 | 0.078 | 1055/1106 | 0.248 | 0.270 | 1.12 | 0.099 |
| C14 | rs7158073 | 99211442 | T | 719/1159 | 0.429 | 0.461 | 1.14 | 0.058 | 490/449 | 0.458 | 0.450 | 0.97 | 0.72 |
| C05 | rs254850 | 158599309 | T | | | | | | | | | | |
| C12 | rs2417821 | 20081535 | T | 725/1186 | 0.720 | 0.721 | 1.01 | 0.93 | 1042/1100 | 0.668 | 0.670 | 1.01 | 0.91 |
| C04 | rs7661204 | 45728007 | T | 720/1183 | 0.411 | 0.435 | 1.10 | 0.14 | | | | | |
| C05 | rs4921437 | 158623529 | T | 725/1194 | 0.197 | 0.194 | 0.98 | 0.8 | 1045/1092 | 0.171 | 0.178 | 1.05 | 0.56 |
| C05 | rs832540 | 56234959 | C | 711/1175 | 0.370 | 0.385 | 1.06 | 0.36 | 1052/1102 | 0.372 | 0.365 | 0.97 | 0.64 |
| C07 | rs324594 | 136305863 | C | | | | | | | | | | |
| C20 | rs1741318 | 4105600 | A | | | | | | | | | | |
| C17 | rs9902941 | 17674485 | C | 736/1150 | 0.355 | 0.378 | 1.10 | 0.15 | | | | | |
| C05 | rs7709212 | 158696755 | T | | | | | | | | | | |
| C01 | rs2946534 | 18647370 | T | | | | | | | | | | |
| C05 | rs6556861 | 94554256 | G | 719/1169 | 0.631 | 0.615 | 0.93 | 0.33 | 1056/1099 | 0.652 | 0.658 | 1.03 | 0.66 |
| C16 | rs8050136 | 52373776 | A | | | | | | 1064/1091 | 0.414 | 0.425 | 1.05 | 0.47 |
| C16 | rs3751812 | 52375961 | T | | | | | | 1070/1114 | 0.412 | 0.420 | 1.03 | 0.59 |
| C13 | rs4769613 | 28036609 | T | 733/1190 | 0.458 | 0.463 | 1.02 | 0.77 | 1052/1090 | 0.498 | 0.488 | 0.96 | 0.51 |
| C10 | rs2243548 | 91000293 | A | | | | | | | | | | |
| C19 | rs12459084 | 17031885 | T | 732/1196 | 0.413 | 0.409 | 0.98 | 0.82 | | | | | |
| C06 | rs2074464 | 29516507 | T | 728/1184 | 0.451 | 0.466 | 1.06 | 0.37 | 1034/1082 | 0.462 | 0.448 | 0.94 | 0.36 |
| C02 | rs2244871 | 128115310 | C | 563/924 | 0.388 | 0.439 | 1.24 | 0.0059 | | | | | |
| C05 | rs270654 | 158497687 | G | | | | | | | | | | |
| C17 | rs854787 | 17975744 | C | 732/1180 | 0.413 | 0.438 | 1.11 | 0.13 | 1017/1043 | 0.501 | 0.490 | 0.96 | 0.48 |
| C11 | rs7944761 | 1361414 | T | | | | | | | | | | |
| C15 | rs4779984 | 30302218 | G | | | | | | | | | | |
| C17 | rs6502622 | 17775416 | G | 734/1150 | 0.377 | 0.400 | 1.10 | 0.17 | 1048/1098 | 0.456 | 0.443 | 0.95 | 0.41 |
| C17 | rs3183702 | 17688014 | T | 572/926 | 0.355 | 0.369 | 1.06 | 0.44 | 1059/1099 | 0.435 | 0.421 | 0.95 | 0.37 |
| C05 | rs1433048 | 158688423 | G | | | | | | | | | | |
| C17 | rs4925119 | 17683629 | A | 729/1173 | 0.350 | 0.374 | 1.11 | 0.13 | 1026/1082 | 0.437 | 0.423 | 0.95 | 0.36 |
| C01 | rs2476601 | 114179091 | A | 572/921 | 0.087 | 0.097 | 1.14 | 0.33 | 1049/1107 | 0.062 | 0.067 | 1.08 | 0.52 |
| C05 | rs870347 | 6898035 | T | | | | | | | | | | |
| C08 | rs334198 | 18933089 | C | | | | | | | | | | |
| C17 | rs854813 | 17944570 | C | 209/412 | 0.392 | 0.417 | 1.11 | 0.39 | 1057/1102 | 0.502 | 0.471 | 0.88 | 0.036 |
| C06 | rs7753765 | 71732495 | A | | | | | | | | | | |
| C17 | rs4925114 | 17651995 | A | 211/413 | 0.353 | 0.389 | 1.16 | 0.22 | | | | | |
| C05 | rs270661 | 158492732 | A | 736/1196 | 0.209 | 0.189 | 0.89 | 0.15 | 1066/1112 | 0.194 | 0.188 | 0.96 | 0.63 |
| C05 | rs953861 | 158705160 | G | | | | | | | | | | |
| C05 | rs10045431 | 158747111 | A | | | | | | | | | | |
| C14 | rs8003722 | 68388015 | A | 735/1186 | 0.144 | 0.136 | 0.94 | 0.48 | | | | | |
| C09 | rs2297538 | 138684489 | G | 736/1193 | 0.798 | 0.795 | 0.98 | 0.79 | 712/386 | 0.754 | 0.750 | 0.98 | 0.83 |
| C02 | rs12329252 | 212093968 | T | 737/1197 | 0.659 | 0.665 | 1.03 | 0.69 | 1047/1095 | 0.713 | 0.688 | 0.88 | 0.065 |
| C01 | rs3748744 | 19828833 | G | | | | | | | | | | |
| C05 | rs4704400 | 76586766 | T | | | | | | | | | | |
| C08 | rs3102526 | 96858538 | G | | | | | | | | | | |
| C15 | rs2110209 | 24946433 | A | 737/1191 | 0.035 | 0.032 | 0.93 | 0.7 | | | | | |
| C16 | rs1870843 | 81316815 | T | | | | | | | | | | |
| C08 | rs3134517 | 96859237 | G | | | | | | | | | | |
| C21 | rs2828495 | 24038081 | A | | | | | | | | | | |
| C04 | rs4282162 | 21426819 | T | 732/1198 | 0.488 | 0.488 | 1.00 | 0.99 | | | | | |
| C17 | rs2955355 | 17889200 | C | 212/413 | 0.302 | 0.337 | 1.17 | 0.21 | 1052/1055 | 0.402 | 0.369 | 0.87 | 0.028 |
| C12 | rs11045689 | 21092930 | G | 734/1195 | 0.328 | 0.365 | 1.18 | 0.019 | | | | | |
| C05 | rs7447732 | 158461713 | C | 732/1198 | 0.328 | 0.314 | 0.94 | 0.37 | 1067/1100 | 0.305 | 0.282 | 0.90 | 0.11 |
| C18 | rs1365254 | 70238283 | T | | | | | | | | | | |
| C13 | rs1887263 | 22381716 | A | | | | | | | | | | |
| C12 | rs12304836 | 124654446 | C | 729/1172 | 0.147 | 0.145 | 0.99 | 0.91 | | | | | |
| C05 | rs38055 | 52596401 | T | 729/1194 | 0.306 | 0.311 | 1.02 | 0.75 | 1054/1102 | 0.316 | 0.305 | 0.95 | 0.45 |
| C04 | rs4697653 | 10337857 | G | 723/1187 | 0.558 | 0.540 | 0.93 | 0.27 | 1019/1076 | 0.546 | 0.571 | 1.11 | 0.097 |
| C05 | rs728676 | 118087433 | T | 718/1152 | 0.493 | 0.486 | 0.97 | 0.66 | | | | | |
| C06 | rs6905466 | 19958854 | T | | | | | | | | | | |
| C10 | rs11199158 | 85297471 | G | 734/1195 | 0.636 | 0.638 | 1.01 | 0.89 | 1049/1093 | 0.653 | 0.655 | 1.01 | 0.86 |
| C01 | rs4845552 | 151746622 | G | 736/1197 | 0.140 | 0.154 | 1.12 | 0.24 | 1060/1105 | 0.153 | 0.134 | 0.86 | 0.076 |
| C02 | rs1395930 | 123468570 | T | 737/1188 | 0.507 | 0.508 | 1.00 | 0.97 | 1037/1085 | 0.548 | 0.565 | 1.07 | 0.26 |
| C04 | rs6534683 | 129601758 | T | | | | | | | | | | |
| C02 | rs6747236 | 35901725 | T | 432/1102 | 0.760 | 0.774 | 1.08 | 0.44 | | | | | |
| C06 | rs3016536 | 162157939 | G | | | | | | | | | | |
| C10 | rs12269612 | 15475877 | T | 734/1196 | 0.382 | 0.361 | 0.91 | 0.19 | 1055/1088 | 0.382 | 0.353 | 0.88 | 0.049 |
| C17 | rs854793 | 17971249 | A | 213/393 | 0.202 | 0.224 | 1.14 | 0.37 | 1031/1102 | 0.268 | 0.245 | 0.89 | 0.083 |
| C12 | rs3742049 | 119438873 | C | 120/254 | 0.896 | 0.884 | 0.88 | 0.63 | | | | | |
| C01 | rs2991515 | 57656898 | T | | | | | | | | | | |
| C18 | rs4271662 | 26051291 | C | 736/1194 | 0.596 | 0.601 | 1.02 | 0.76 | 1045/1075 | 0.644 | 0.633 | 0.95 | 0.44 |
| C06 | rs7739802 | 162154323 | T | | | | | | | | | | |
| C08 | rs10504537 | 73402234 | A | | | | | | | | | | |
| C14 | rs17776453 | 100774793 | G | | | | | | | | | | |
| C10 | rs7086224 | 30857283 | A | | | | | | | | | | |
| C01 | rs12724604 | 20948045 | A | | | | | | | | | | |
| C03 | rs9874646 | 62437858 | T | 735/1199 | 0.686 | 0.681 | 0.98 | 0.74 | | | | | |
| C05 | rs950195 | 97466690 | G | 727/1193 | 0.646 | 0.633 | 0.94 | 0.39 | 1036/1086 | 0.647 | 0.622 | 0.90 | 0.095 |
| C14 | rs2281519 | 93846385 | T | | | | | | | | | | |
| C10 | rs4880711 | 5217871 | C | | | | | | | | | | |
| C08 | rs3931282 | 129179963 | T | 732/1182 | 0.524 | 0.542 | 1.08 | 0.27 | | | | | |
| C10 | rs11198993 | 85244450 | A | 732/1192 | 0.774 | 0.759 | 0.92 | 0.3 | 1048/1096 | 0.776 | 0.778 | 1.01 | 0.87 |
| C12 | rs1568383 | 88017285 | T | | | | | | | | | | |
| C06 | rs9380681 | 37638433 | C | | | | | | | | | | |
| C02 | rs6709904 | 43933828 | A | 682/1110 | 0.891 | 0.885 | 0.94 | 0.58 | | | | | |
| C10 | rs2279434 | 45275070 | T | | | | | | | | | | |
| C13 | rs1375719 | 102208783 | C | 116/250 | 0.504 | 0.464 | 0.85 | 0.31 | | | | | |
| C10 | rs7921473 | 85156395 | G | 733/1177 | 0.840 | 0.828 | 0.91 | 0.3 | 1064/1104 | 0.823 | 0.819 | 0.97 | 0.73 |
| C14 | rs1957860 | 53499105 | T | 737/1198 | 0.455 | 0.483 | 1.12 | 0.082 | | | | | |
| C02 | rs3943703 | 127613790 | C | | | | | | | | | | |
| C08 | rs7006687 | 18277862 | T | 730/1196 | 0.567 | 0.549 | 0.93 | 0.27 | 1056/1094 | 0.561 | 0.521 | 0.85 | 0.0077 |
| C12 | rs1465026 | 65789635 | G | 731/1189 | 0.837 | 0.821 | 0.90 | 0.21 | 1061/1118 | 0.842 | 0.842 | 1.00 | 0.97 |
| C14 | rs4444235 | 53480669 | T | 733/1186 | 0.548 | 0.527 | 0.92 | 0.22 | | | | | |
| C02 | rs921083 | 176453128 | G | 734/1199 | 0.809 | 0.817 | 1.06 | 0.5 | 1057/1096 | 0.828 | 0.828 | 1.00 | 0.99 |
| C08 | rs4732849 | 28249765 | T | 724/1188 | 0.330 | 0.314 | 0.93 | 0.3 | | | | | |

| Table 4E - MI | | | | | | | | |
|---|---|---|---|---|---|---|---|---|
| | | Pos in | | Hopkins (1567/184) | | | | |
| Chr | SNP | Build 36 | Allele | nc/na | Ctr. fr | Aff. fr | OR | P |
| C06 | rs11751605 | 160883220 | C | 1565/184 | 0.155 | 0.179 | 1.19 | 0.23 |
| C10 | rs2243547 | 91000459 | G | 1539/184 | 0.284 | 0.323 | 1.21 | 0.12 |
| C10 | rs1412444 | 90992907 | A | 1565/183 | 0.321 | 0.344 | 1.11 | 0.38 |
| C01 | rs12134779 | 63168930 | T | 1567/183 | 0.265 | 0.281 | 1.08 | 0.52 |
| C11 | rs2163612 | 82856844 | G | 1566/182 | 0.497 | 0.489 | 0.97 | 0.78 |
| C05 | rs254850 | 158599309 | T | 1567/184 | 0.195 | 0.190 | 0.97 | 0.83 |
| C17 | rs9902941 | 17674485 | C | 1567/183 | 0.367 | 0.350 | 0.93 | 0.51 |
| C20 | rs6076623 | 4084671 | T | 1567/183 | 0.296 | 0.325 | 1.15 | 0.25 |
| C05 | rs4921437 | 158623529 | T | 1566/183 | 0.184 | 0.189 | 1.03 | 0.84 |
| C05 | rs832540 | 56234959 | C | 1567/184 | 0.361 | 0.397 | 1.16 | 0.18 |
| C19 | rs12459084 | 17031885 | T | 1512/182 | 0.415 | 0.434 | 1.08 | 0.48 |
| C07 | rs1029396 | 116812056 | G | 1566/183 | 0.080 | 0.115 | 1.50 | 0.027 |
| C17 | rs6502622 | 17775416 | G | 1563/184 | 0.401 | 0.386 | 0.94 | 0.59 |
| C17 | rs854787 | 17975744 | C | 1552/181 | 0.428 | 0.406 | 0.92 | 0.43 |
| C14 | rs7158073 | 99211442 | T | 1509/180 | 0.431 | 0.428 | 0.99 | 0.9 |
| C10 | rs2243548 | 91000293 | A | 1566/183 | 0.504 | 0.544 | 1.17 | 0.15 |
| C17 | rs4925119 | 17683629 | A | 1542/181 | 0.361 | 0.340 | 0.91 | 0.43 |
| C17 | rs3183702 | 17688014 | T | 1561/183 | 0.365 | 0.344 | 0.92 | 0.44 |
| C17 | rs854813 | 17944570 | C | 1551/180 | 0.409 | 0.381 | 0.89 | 0.3 |
| C05 | rs953861 | 158705160 | G | 1565/184 | 0.168 | 0.168 | 1.00 | 1 |
| C07 | rs12534186 | 117079572 | T | 1567/184 | 0.095 | 0.125 | 1.36 | 0.077 |
| C12 | rs2417821 | 20081535 | T | 1566/184 | 0.728 | 0.723 | 0.97 | 0.82 |
| C20 | rs1741318 | 4105600 | A | 1566/183 | 0.377 | 0.383 | 1.02 | 0.83 |
| C06 | rs2074464 | 29516507 | T | 1562/183 | 0.447 | 0.426 | 0.92 | 0.46 |
| C07 | rs324594 | 136305863 | C | 1567/183 | 0.269 | 0.240 | 0.86 | 0.23 |
| C05 | rs270654 | 158497687 | G | 1567/183 | 0.108 | 0.096 | 0.88 | 0.48 |
| C15 | rs4779984 | 30302218 | G | 1562/183 | 0.125 | 0.134 | 1.08 | 0.64 |
| C05 | rs7709212 | 158696755 | T | 1567/183 | 0.674 | 0.699 | 1.12 | 0.33 |
| C05 | rs10045431 | 158747111 | A | 1567/184 | 0.289 | 0.299 | 1.05 | 0.7 |
| C17 | rs2955355 | 17889200 | C | 1565/183 | 0.318 | 0.306 | 0.95 | 0.65 |
| C11 | rs7944761 | 1361414 | T | 1567/184 | 0.563 | 0.571 | 1.03 | 0.79 |
| C05 | rs4704400 | 76586766 | T | 1563/183 | 0.485 | 0.503 | 1.07 | 0.52 |
| C17 | rs4925114 | 17651995 | A | 1561/183 | 0.372 | 0.344 | 0.89 | 0.3 |
| C05 | rs1433048 | 158688423 | G | 1565/183 | 0.184 | 0.172 | 0.92 | 0.56 |
| C05 | rs7447732 | 158461713 | C | 1566/184 | 0.321 | 0.323 | 1.01 | 0.92 |
| C02 | rs2244871 | 128115310 | C | 1556/182 | 0.426 | 0.396 | 0.88 | 0.26 |
| C01 | rs2946534 | 18647370 | T | 1564/183 | 0.548 | 0.552 | 1.02 | 0.88 |
| C04 | rs7661204 | 45728007 | T | 1567/183 | 0.410 | 0.385 | 0.90 | 0.36 |
| C05 | rs6556861 | 94554256 | G | 1567/183 | 0.623 | 0.612 | 0.95 | 0.68 |
| C06 | rs7753765 | 71732495 | A | 1567/184 | 0.170 | 0.188 | 1.13 | 0.4 |
| C05 | rs870347 | 6898035 | T | 1566/184 | 0.940 | 0.946 | 1.11 | 0.66 |
| C10 | rs12269612 | 15475877 | T | 1567/184 | 0.376 | 0.399 | 1.10 | 0.38 |
| C15 | rs2110209 | 24946433 | A | 1567/184 | 0.031 | 0.024 | 0.78 | 0.46 |
| C02 | rs12329252 | 212093968 | T | 1567/183 | 0.657 | 0.678 | 1.10 | 0.44 |
| C14 | rs8003722 | 68388015 | A | 1566/183 | 0.138 | 0.139 | 1.01 | 0.93 |
| C21 | rs2828495 | 24038081 | A | 1566/183 | 0.233 | 0.230 | 0.98 | 0.89 |
| C05 | rs270661 | 158492732 | A | 1567/184 | 0.194 | 0.171 | 0.86 | 0.29 |
| C08 | rs334198 | 18933089 | C | 1566/184 | 0.922 | 0.924 | 1.02 | 0.92 |
| C01 | rs2476601 | 114179091 | A | 1566/183 | 0.091 | 0.096 | 1.06 | 0.77 |
| C13 | rs4769613 | 28036609 | T | 1567/183 | 0.469 | 0.514 | 1.20 | 0.11 |
| C08 | rs3102526 | 96858538 | G | 1565/183 | 0.620 | 0.658 | 1.18 | 0.15 |
| C08 | rs3134517 | 96859237 | G | 1567/184 | 0.620 | 0.658 | 1.18 | 0.15 |
| C01 | rs3748744 | 19828833 | G | 1561/182 | 0.896 | 0.912 | 1.21 | 0.32 |
| C04 | rs4282162 | 21426819 | T | 1567/183 | 0.503 | 0.525 | 1.09 | 0.42 |
| C16 | rs8050136 | 52373776 | A | 1567/183 | 0.411 | 0.402 | 0.96 | 0.72 |
| C16 | rs3751812 | 52375961 | T | 1567/184 | 0.410 | 0.402 | 0.97 | 0.78 |
| C17 | rs854793 | 17971249 | A | 1567/183 | 0.232 | 0.235 | 1.02 | 0.89 |
| C10 | rs4880711 | 5217871 | C | 1562/184 | 0.349 | 0.394 | 1.22 | 0.087 |
| C12 | rs12304836 | 124654446 | C | 1567/184 | 0.151 | 0.155 | 1.03 | 0.84 |
| C05 | rs38055 | 52596401 | T | 1567/184 | 0.340 | 0.315 | 0.89 | 0.34 |
| C09 | rs2297538 | 138684489 | G | 1555/180 | 0.791 | 0.789 | 0.99 | 0.91 |
| C16 | rs1870843 | 81316815 | T | 1567/184 | 0.603 | 0.587 | 0.94 | 0.55 |
| C06 | rs6905466 | 19958854 | T | 1567/183 | 0.338 | 0.342 | 1.02 | 0.89 |
| C12 | rs11045689 | 21092930 | G | 1557/182 | 0.346 | 0.288 | 0.76 | 0.025 |
| C13 | rs1887263 | 22381716 | A | 1567/184 | 0.174 | 0.193 | 1.13 | 0.38 |
| C18 | rs4271662 | 26051291 | C | 1567/183 | 0.595 | 0.617 | 1.10 | 0.41 |
| C18 | rs1365254 | 70238283 | T | 1567/184 | 0.187 | 0.168 | 0.88 | 0.39 |
| C06 | rs3016536 | 162157939 | G | 1567/183 | 0.596 | 0.579 | 0.93 | 0.54 |
| C12 | rs3742049 | 119438873 | C | 1565/183 | 0.885 | 0.888 | 1.03 | 0.86 |
| C03 | rs9874646 | 62437858 | T | 1567/183 | 0.669 | 0.699 | 1.15 | 0.24 |
| C14 | rs2281519 | 93846385 | T | 1564/183 | 0.267 | 0.235 | 0.84 | 0.18 |
| C02 | rs6747236 | 35901725 | T | 1566/184 | 0.755 | 0.726 | 0.86 | 0.21 |
| C04 | rs6534683 | 129601758 | T | 1567/183 | 0.652 | 0.628 | 0.90 | 0.37 |
| C01 | rs2991515 | 57656898 | T | 1565/184 | 0.458 | 0.438 | 0.92 | 0.44 |
| C04 | rs4697653 | 10337857 | G | 1553/182 | 0.567 | 0.569 | 1.01 | 0.95 |
| C06 | rs9380681 | 37638433 | C | 1567/183 | 0.315 | 0.328 | 1.06 | 0.62 |
| C10 | rs11199158 | 85297471 | G | 1566/183 | 0.645 | 0.623 | 0.91 | 0.41 |
| C13 | rs1375719 | 102208783 | C | 1566/183 | 0.492 | 0.516 | 1.10 | 0.37 |
| C08 | rs3931282 | 129179963 | T | 1567/184 | 0.517 | 0.514 | 0.99 | 0.9 |
| C14 | rs17776453 | 100774793 | G | 1567/183 | 0.956 | 0.967 | 1.37 | 0.28 |
| C01 | rs12724604 | 20948045 | A | 1566/184 | 0.044 | 0.019 | 0.42 | 0.011 |
| C05 | rs950195 | 97466690 | G | 1565/184 | 0.654 | 0.679 | 1.12 | 0.34 |
| C05 | rs728676 | 118087433 | T | 1567/183 | 0.462 | 0.429 | 0.87 | 0.22 |
| C06 | rs7739802 | 162154323 | T | 1567/183 | 0.549 | 0.530 | 0.92 | 0.48 |
| C10 | rs7086224 | 30857283 | A | 1567/184 | 0.418 | 0.459 | 1.18 | 0.13 |
| C01 | rs4845552 | 151746622 | G | 1567/183 | 0.125 | 0.107 | 0.83 | 0.3 |
| C08 | rs10504537 | 73402234 | A | 1567/184 | 0.866 | 0.851 | 0.88 | 0.43 |
| C02 | rs1395930 | 123468570 | T | 1567/183 | 0.539 | 0.481 | 0.79 | 0.035 |
| C02 | rs3943703 | 127613790 | C | 1558/181 | 0.228 | 0.240 | 1.07 | 0.61 |
| C10 | rs2279434 | 45275070 | T | 1567/184 | 0.075 | 0.065 | 0.86 | 0.48 |
| C10 | rs11198993 | 85244450 | A | 1566/183 | 0.786 | 0.790 | 1.02 | 0.86 |
| C10 | rs7921473 | 85156395 | G | 1567/183 | 0.844 | 0.863 | 1.17 | 0.33 |
| C12 | rs1465026 | 65789635 | G | 1564/184 | 0.836 | 0.832 | 0.97 | 0.81 |
| C12 | rs1568383 | 88017285 | T | 1567/183 | 0.117 | 0.134 | 1.17 | 0.36 |
| C08 | rs4732849 | 28249765 | T | 1566/184 | 0.310 | 0.318 | 1.04 | 0.76 |
| C08 | rs7006687 | 18277862 | T | 1567/183 | 0.558 | 0.557 | 1.00 | 0.99 |
| C02 | rs921083 | 176453128 | G | 1567/182 | 0.820 | 0.788 | 0.82 | 0.15 |
| C02 | rs6709904 | 43933828 | A | 1561/184 | 0.884 | 0.856 | 0.78 | 0.12 |
| C14 | rs1957860 | 53499105 | T | 1567/183 | 0.474 | 0.459 | 0.94 | 0.57 |
| C14 | rs4444235 | 53480669 | T | 1567/183 | 0.546 | 0.552 | 1.02 | 0.83 |

| | |
|---|---|
| WTCCC (14246/1926) | Replication (19396/5182) |

| Chr | nc/na | Ctr. fr | Aff. fr | OR | P | OR (99% CI) | P |
|---|---|---|---|---|---|---|---|
| C06 | 14237/1924 | 0.173 | 0.197 | 1.17 | 0.00026 | 1.15 (1.08-1.22) | 2.87E−05 |
| C10 | 14223/1921 | 0.316 | 0.327 | 1.05 | 0.15 | 1.07 (1.00-1.14) | 0.067 |
| C10 | | | | | | 1.09 (1.01-1.18) | 0.026 |
| C01 | 14220/1921 | 0.251 | 0.254 | 1.01 | 0.77 | 1.04 (0.99-1.10) | 0.13 |
| C11 | 13841/1882 | 0.482 | 0.488 | 1.02 | 0.53 | 1.03 (0.97-1.08) | 0.34 |
| C05 | 14239/1924 | 0.201 | 0.217 | 1.10 | 0.022 | 1.09 (1.01-1.18) | 0.033 |
| C17 | 14239/1924 | 0.350 | 0.347 | 0.99 | 0.7 | 1.03 (0.97-1.08) | 0.34 |
| C20 | 14220/1922 | 0.312 | 0.315 | 1.01 | 0.77 | 1.03 (0.98-1.09) | 0.2 |
| C05 | 14243/1925 | 0.182 | 0.196 | 1.09 | 0.046 | 1.06 (0.99-1.13) | 0.11 |
| C05 | 14239/1925 | 0.354 | 0.371 | 1.08 | 0.037 | 1.06 (1.01-1.12) | 0.032 |
| C19 | | | | | | 1.01 (0.90-1.13) | 0.86 |
| C07 | 14243/1924 | 0.084 | 0.089 | 1.06 | 0.32 | 1.09 (1.00-1.19) | 0.062 |
| C17 | 14241/1925 | 0.375 | 0.370 | 0.98 | 0.53 | 1.02 (0.97-1.07) | 0.41 |
| C17 | 14215/1921 | 0.405 | 0.402 | 0.99 | 0.75 | 1.03 (0.98-1.08) | 0.27 |
| C14 | | | | | | 1.04 (0.96-1.13) | 0.31 |
| C10 | 14219/1922 | 0.520 | 0.528 | 1.03 | 0.36 | 1.04 (0.98-1.11) | 0.2 |
| C17 | 14243/1925 | 0.350 | 0.346 | 0.99 | 0.69 | 1.02 (0.97-1.07) | 0.46 |
| C17 | 14242/1924 | 0.348 | 0.344 | 0.98 | 0.61 | 1.01 (0.96-1.07) | 0.62 |
| C17 | 14242/1925 | 0.394 | 0.395 | 1.00 | 0.9 | 1.02 (0.96-1.07) | 0.57 |
| C05 | 14242/1924 | 0.164 | 0.174 | 1.07 | 0.12 | 1.07 (0.98-1.16) | 0.14 |
| C07 | 14241/1925 | 0.096 | 0.099 | 1.03 | 0.62 | 1.06 (0.95-1.18) | 0.31 |
| C12 | 14243/1925 | 0.717 | 0.736 | 1.10 | 0.014 | 1.06 (1.00-1.12) | 0.053 |
| C20 | 14212/1921 | 0.397 | 0.407 | 1.04 | 0.21 | 1.04 (0.98-1.11) | 0.21 |
| C06 | 14241/1924 | 0.431 | 0.445 | 1.06 | 0.12 | 1.03 (0.98-1.09) | 0.2 |
| C07 | 14237/1924 | 0.252 | 0.263 | 1.06 | 0.14 | 1.04 (0.97-1.12) | 0.29 |
| C05 | 14228/1919 | 0.106 | 0.116 | 1.10 | 0.069 | 1.08 (0.98-1.20) | 0.12 |
| C15 | 14222/1922 | 0.074 | 0.076 | 1.04 | 0.54 | 1.05 (0.93-1.18) | 0.46 |
| C05 | 14228/1923 | 0.628 | 0.636 | 1.04 | 0.32 | 1.04 (0.98-1.12) | 0.22 |
| C05 | 14240/1924 | 0.290 | 0.301 | 1.06 | 0.13 | 1.06 (0.99-1.13) | 0.12 |
| C17 | 14240/1925 | 0.310 | 0.301 | 0.96 | 0.24 | 0.99 (0.94-1.05) | 0.72 |
| C11 | 14211/1921 | 0.510 | 0.520 | 1.04 | 0.28 | 1.04 (0.97-1.11) | 0.26 |
| C05 | 14240/1925 | 0.483 | 0.491 | 1.03 | 0.33 | 1.04 (0.98-1.10) | 0.24 |
| C17 | 14222/1922 | 0.333 | 0.330 | 0.98 | 0.63 | 1.00 (0.94-1.06) | 0.96 |
| C05 | 14233/1923 | 0.178 | 0.187 | 1.06 | 0.18 | 1.05 (0.96-1.14) | 0.27 |
| C05 | 14232/1924 | 0.301 | 0.314 | 1.07 | 0.084 | 1.03 (0.97-1.08) | 0.32 |
| C02 | 14218/1921 | 0.441 | 0.448 | 1.03 | 0.4 | 1.03 (0.98-1.09) | 0.22 |
| C01 | 14219/1922 | 0.560 | 0.560 | 1.00 | 0.97 | 1.00 (0.94-1.06) | 0.99 |
| C04 | 14242/1924 | 0.449 | 0.437 | 0.95 | 0.14 | 0.97 (0.92-1.03) | 0.39 |
| C05 | 14236/1924 | 0.607 | 0.628 | 1.09 | 0.013 | 1.03 (0.97-1.09) | 0.29 |
| C06 | 14241/1925 | 0.157 | 0.154 | 0.97 | 0.55 | 1.00 (0.92-1.08) | 0.91 |
| C05 | 14242/1925 | 0.945 | 0.946 | 1.03 | 0.68 | 1.04 (0.90-1.20) | 0.59 |
| C10 | 14240/1925 | 0.373 | 0.390 | 1.08 | 0.035 | 1.03 (0.97-1.08) | 0.35 |
| C15 | 14237/1925 | 0.058 | 0.059 | 1.02 | 0.78 | 1.00 (0.88-1.14) | 0.98 |
| C02 | 14239/1925 | 0.656 | 0.667 | 1.05 | 0.19 | 1.02 (0.97-1.08) | 0.4 |
| C14 | 14075/1906 | 0.153 | 0.162 | 1.07 | 0.18 | 1.04 (0.96-1.12) | 0.34 |
| C21 | 14236/1924 | 0.230 | 0.231 | 1.00 | 0.97 | 1.00 (0.93-1.08) | 0.99 |
| C05 | 14246/1925 | 0.193 | 0.212 | 1.13 | 0.0051 | 1.03 (0.96-1.10) | 0.41 |
| C08 | 14241/1924 | 0.928 | 0.920 | 0.89 | 0.084 | 0.94 (0.84-1.04) | 0.23 |
| C01 | 14238/1924 | 0.121 | 0.108 | 0.88 | 0.02 | 0.97 (0.89-1.05) | 0.45 |
| C13 | 14214/1921 | 0.474 | 0.471 | 0.99 | 0.72 | 1.00 (0.95-1.05) | 1 |
| C08 | 14242/1925 | 0.623 | 0.625 | 1.01 | 0.83 | 1.02 (0.96-1.09) | 0.53 |
| C08 | 14241/1925 | 0.623 | 0.625 | 1.01 | 0.83 | 1.02 (0.96-1.09) | 0.53 |
| C01 | 14236/1925 | 0.903 | 0.907 | 1.05 | 0.38 | 1.07 (0.95-1.19) | 0.26 |
| C04 | 14242/1924 | 0.506 | 0.505 | 0.99 | 0.84 | 1.00 (0.94-1.06) | 0.97 |
| C16 | 14192/1918 | 0.407 | 0.406 | 1.00 | 0.96 | 1.01 (0.95-1.07) | 0.81 |
| C16 | 14243/1925 | 0.406 | 0.406 | 1.00 | 0.99 | 1.01 (0.95-1.06) | 0.82 |
| C17 | 14222/1922 | 0.260 | 0.250 | 0.95 | 0.2 | 0.98 (0.92-1.04) | 0.51 |
| C10 | 14241/1925 | 0.377 | 0.380 | 1.01 | 0.73 | 1.03 (0.96-1.10) | 0.4 |
| C12 | 14234/1924 | 0.137 | 0.131 | 0.95 | 0.34 | 0.99 (0.92-1.07) | 0.81 |
| C05 | 14240/1924 | 0.317 | 0.319 | 1.01 | 0.82 | 1.02 (0.97-1.08) | 0.35 |
| C09 | | | | | | 0.93 (0.84-1.04) | 0.2 |
| C16 | 14224/1922 | 0.637 | 0.638 | 1.00 | 0.91 | 1.00 (0.93-1.07) | 0.94 |
| C06 | 14224/1922 | 0.331 | 0.326 | 0.98 | 0.55 | 0.98 (0.92-1.05) | 0.6 |
| C12 | 14235/1924 | 0.356 | 0.358 | 1.01 | 0.84 | 1.01 (0.96-1.07) | 0.75 |
| C13 | 14241/1925 | 0.176 | 0.177 | 1.01 | 0.79 | 1.02 (0.94-1.11) | 0.6 |
| C18 | 14226/1922 | 0.597 | 0.599 | 1.01 | 0.81 | 1.01 (0.97-1.07) | 0.56 |
| C18 | 13709/1832 | 0.188 | 0.192 | 1.03 | 0.52 | 1.02 (0.93-1.10) | 0.72 |
| C06 | 14219/1921 | 0.628 | 0.628 | 1.00 | 0.99 | 0.99 (0.93-1.06) | 0.87 |
| C12 | 14239/1924 | 0.897 | 0.898 | 1.01 | 0.88 | 1.01 (0.92-1.11) | 0.85 |
| C03 | 14223/1921 | 0.670 | 0.669 | 0.99 | 0.84 | 1.01 (0.96-1.07) | 0.63 |
| C14 | 14234/1923 | 0.274 | 0.268 | 0.97 | 0.41 | 0.96 (0.89-1.03) | 0.24 |
| C02 | 14241/1924 | 0.762 | 0.754 | 0.96 | 0.29 | 0.97 (0.91-1.03) | 0.35 |
| C04 | 14230/1923 | 0.671 | 0.674 | 1.02 | 0.68 | 1.00 (0.94-1.08) | 0.9 |
| C01 | 14219/1921 | 0.399 | 0.392 | 0.97 | 0.38 | 0.97 (0.92-1.03) | 0.37 |
| C04 | 14229/1923 | 0.540 | 0.533 | 0.97 | 0.4 | 0.98 (0.93-1.03) | 0.43 |
| C06 | 14229/1923 | 0.365 | 0.362 | 0.99 | 0.73 | 0.99 (0.93-1.06) | 0.85 |
| C10 | 14246/1925 | 0.650 | 0.650 | 1.00 | 0.99 | 0.99 (0.94-1.05) | 0.84 |
| C13 | 14222/1922 | 0.510 | 0.510 | 1.00 | 0.95 | 0.99 (0.94-1.05) | 0.84 |
| C08 | 14236/1924 | 0.533 | 0.536 | 1.01 | 0.74 | 1.03 (0.98-1.09) | 0.28 |
| C14 | 14231/1923 | 0.923 | 0.923 | 1.00 | 0.97 | 1.01 (0.90-1.14) | 0.85 |
| C01 | 14244/1925 | 0.041 | 0.040 | 0.96 | 0.66 | 0.92 (0.78-1.08) | 0.3 |
| C05 | 14236/1924 | 0.649 | 0.647 | 0.99 | 0.82 | 0.97 (0.92-1.03) | 0.34 |
| C05 | 14223/1923 | 0.484 | 0.471 | 0.95 | 0.14 | 0.97 (0.92-1.02) | 0.28 |
| C06 | 14219/1922 | 0.624 | 0.620 | 0.98 | 0.64 | 0.98 (0.92-1.04) | 0.51 |
| C10 | 14217/1921 | 0.450 | 0.441 | 0.96 | 0.26 | 0.98 (0.92-1.04) | 0.53 |
| C01 | 14237/1924 | 0.139 | 0.135 | 0.97 | 0.51 | 0.95 (0.88-1.03) | 0.23 |
| C08 | 14233/1923 | 0.871 | 0.868 | 0.98 | 0.68 | 0.97 (0.88-1.07) | 0.53 |
| C02 | 14242/1925 | 0.498 | 0.495 | 0.99 | 0.71 | 0.98 (0.93-1.02) | 0.32 |
| C02 | 14171/1913 | 0.233 | 0.222 | 0.94 | 0.14 | 0.95 (0.88-1.03) | 0.21 |
| C10 | 14239/1924 | 0.085 | 0.081 | 0.95 | 0.42 | 0.92 (0.83-1.02) | 0.11 |
| C10 | 14241/1924 | 0.780 | 0.772 | 0.95 | 0.23 | 0.97 (0.91-1.03) | 0.28 |
| C10 | 14241/1923 | 0.857 | 0.845 | 0.91 | 0.05 | 0.93 (0.86-1.00) | 0.054 |
| C12 | 14240/1925 | 0.836 | 0.829 | 0.95 | 0.3 | 0.96 (0.89-1.03) | 0.21 |
| C12 | 14238/1924 | 0.111 | 0.104 | 0.92 | 0.14 | 0.93 (0.85-1.02) | 0.12 |
| C08 | 14240/1926 | 0.328 | 0.315 | 0.94 | 0.11 | 0.96 (0.91-1.02) | 0.16 |
| C08 | 14221/1922 | 0.556 | 0.550 | 0.97 | 0.44 | 0.94 (0.89-1.00) | 0.034 |
| C02 | 14215/1922 | 0.731 | 0.717 | 0.93 | 0.061 | 0.95 (0.90-1.01) | 0.099 |
| C02 | 14233/1924 | 0.900 | 0.896 | 0.96 | 0.47 | 0.94 (0.85-1.03) | 0.19 |
| C14 | 14238/1923 | 0.473 | 0.482 | 1.03 | 0.32 | 1.03 (0.98-1.08) | 0.26 |
| C14 | 14232/1923 | 0.537 | 0.536 | 0.99 | 0.86 | 0.98 (0.93-1.03) | 0.36 |

Table 4F - CAD

| | |
|---|---|
| Pos in Build | Hopkins (1567/389) |

| Chr | SNP | 36 | Allele | nc/na | Ctr. fr | Aff. fr | OR | P |
|---|---|---|---|---|---|---|---|---|
| C06 | rs11751605 | 160883220 | C | 1565/389 | 0.155 | 0.180 | 1.20 | 0.093 |
| C20 | rs6076623 | 4084671 | T | 1567/388 | 0.296 | 0.331 | 1.18 | 0.059 |
| C10 | rs1412444 | 90992907 | A | 1565/388 | 0.321 | 0.345 | 1.11 | 0.2 |
| C11 | rs2163612 | 82856844 | G | 1566/387 | 0.497 | 0.506 | 1.04 | 0.63 |
| C07 | rs1029396 | 116812056 | G | 1566/388 | 0.080 | 0.110 | 1.42 | 0.0092 |
| C10 | rs2243547 | 91000459 | G | 1539/387 | 0.284 | 0.317 | 1.17 | 0.076 |
| C07 | rs12534186 | 117079572 | T | 1567/389 | 0.095 | 0.112 | 1.20 | 0.17 |
| C01 | rs12134779 | 63168930 | T | 1567/388 | 0.265 | 0.278 | 1.07 | 0.47 |
| C14 | rs7158073 | 99211442 | T | 1509/379 | 0.431 | 0.437 | 1.02 | 0.79 |
| C05 | rs254850 | 158599309 | T | 1567/389 | 0.195 | 0.190 | 0.97 | 0.76 |
| C12 | rs2417821 | 20081535 | T | 1566/389 | 0.728 | 0.729 | 1.00 | 0.98 |
| C04 | rs7661204 | 45728007 | T | 1567/388 | 0.410 | 0.374 | 0.86 | 0.064 |
| C05 | rs4921437 | 158623529 | T | 1566/388 | 0.184 | 0.182 | 0.98 | 0.87 |
| C05 | rs832540 | 56234959 | C | 1567/389 | 0.361 | 0.365 | 1.02 | 0.84 |
| C07 | rs324594 | 136305863 | C | 1567/387 | 0.269 | 0.267 | 0.99 | 0.92 |
| C20 | rs1741318 | 4105600 | A | 1566/388 | 0.377 | 0.374 | 0.99 | 0.88 |
| C17 | rs9902941 | 17674485 | C | 1567/387 | 0.367 | 0.368 | 1.00 | 0.96 |
| C05 | rs7709212 | 158696755 | T | 1567/388 | 0.674 | 0.700 | 1.13 | 0.17 |
| C01 | rs2946534 | 18647370 | T | 1564/388 | 0.548 | 0.581 | 1.15 | 0.092 |
| C05 | rs6556861 | 94554256 | G | 1567/388 | 0.623 | 0.610 | 0.94 | 0.48 |
| C16 | rs8050136 | 52373776 | A | 1567/388 | 0.411 | 0.418 | 1.03 | 0.75 |
| C16 | rs3751812 | 52375961 | T | 1567/389 | 0.410 | 0.418 | 1.03 | 0.68 |
| C13 | rs4769613 | 28036609 | T | 1567/388 | 0.469 | 0.514 | 1.20 | 0.024 |
| C10 | rs2243548 | 91000293 | A | 1566/388 | 0.504 | 0.522 | 1.07 | 0.38 |
| C19 | rs12459084 | 17031885 | T | 1512/381 | 0.415 | 0.423 | 1.03 | 0.69 |
| C06 | rs2074464 | 29516507 | T | 1562/388 | 0.447 | 0.452 | 1.02 | 0.77 |
| C02 | rs2244871 | 128115310 | C | 1556/385 | 0.426 | 0.425 | 0.99 | 0.94 |
| C05 | rs270654 | 158497687 | G | 1567/388 | 0.108 | 0.095 | 0.87 | 0.32 |
| C17 | rs854787 | 17975744 | C | 1552/385 | 0.428 | 0.429 | 1.00 | 0.96 |
| C11 | rs7944761 | 1361414 | T | 1567/389 | 0.563 | 0.539 | 0.90 | 0.21 |
| C15 | rs4779984 | 30302218 | G | 1562/388 | 0.125 | 0.121 | 0.96 | 0.76 |
| C17 | rs6502622 | 17775416 | G | 1563/388 | 0.401 | 0.397 | 0.99 | 0.85 |
| C17 | rs3183702 | 17688014 | T | 1561/388 | 0.365 | 0.365 | 1.00 | 0.99 |
| C05 | rs1433048 | 158688423 | G | 1565/388 | 0.184 | 0.178 | 0.96 | 0.67 |
| C17 | rs4925119 | 17683629 | A | 1542/383 | 0.361 | 0.362 | 1.00 | 0.97 |
| C01 | rs2476601 | 114179091 | A | 1566/388 | 0.091 | 0.097 | 1.07 | 0.63 |
| C05 | rs870347 | 6898035 | T | 1566/389 | 0.940 | 0.945 | 1.09 | 0.61 |
| C08 | rs334198 | 18933089 | C | 1566/389 | 0.922 | 0.914 | 0.89 | 0.43 |
| C17 | rs854813 | 17944570 | C | 1551/383 | 0.409 | 0.413 | 1.02 | 0.85 |
| C06 | rs7753765 | 71732495 | A | 1567/389 | 0.170 | 0.163 | 0.95 | 0.66 |
| C17 | rs4925114 | 17651995 | A | 1561/388 | 0.372 | 0.367 | 0.98 | 0.82 |
| C05 | rs270661 | 158492732 | A | 1567/389 | 0.194 | 0.179 | 0.91 | 0.34 |
| C05 | rs953861 | 158705160 | G | 1565/389 | 0.168 | 0.162 | 0.95 | 0.67 |
| C05 | rs10045431 | 158747111 | A | 1567/389 | 0.289 | 0.298 | 1.04 | 0.62 |
| C14 | rs8003722 | 68388015 | A | 1566/388 | 0.138 | 0.146 | 1.07 | 0.57 |
| C09 | rs2297538 | 138684489 | G | 1555/381 | 0.791 | 0.797 | 1.03 | 0.75 |
| C02 | rs12329252 | 212093968 | T | 1567/388 | 0.657 | 0.680 | 1.11 | 0.22 |
| C01 | rs3748744 | 19828833 | G | 1561/387 | 0.896 | 0.898 | 1.03 | 0.85 |
| C05 | rs4704400 | 76586766 | T | 1563/388 | 0.485 | 0.476 | 0.96 | 0.64 |
| C08 | rs3102526 | 96858538 | G | 1565/388 | 0.620 | 0.628 | 1.03 | 0.69 |
| C15 | rs2110209 | 24946433 | A | 1567/389 | 0.031 | 0.030 | 0.94 | 0.8 |
| C16 | rs1870843 | 81316815 | T | 1567/389 | 0.603 | 0.603 | 1.00 | 0.99 |
| C08 | rs3134517 | 96859237 | G | 1567/389 | 0.620 | 0.627 | 1.03 | 0.7 |
| C21 | rs2828495 | 24038081 | A | 1566/388 | 0.233 | 0.201 | 0.83 | 0.056 |
| C04 | rs4282162 | 21426819 | T | 1567/388 | 0.503 | 0.503 | 1.00 | 1 |
| C17 | rs2955355 | 17889200 | C | 1565/388 | 0.318 | 0.326 | 1.04 | 0.65 |
| C12 | rs11045689 | 21092930 | G | 1557/386 | 0.346 | 0.325 | 0.91 | 0.26 |
| C05 | rs7447732 | 158461713 | C | 1566/389 | 0.321 | 0.319 | 0.99 | 0.91 |
| C18 | rs1365254 | 70238283 | T | 1567/389 | 0.187 | 0.180 | 0.96 | 0.67 |
| C13 | rs1887263 | 22381716 | A | 1567/389 | 0.174 | 0.180 | 1.04 | 0.71 |
| C12 | rs12304836 | 124654446 | C | 1567/389 | 0.151 | 0.138 | 0.90 | 0.34 |
| C05 | rs38055 | 52596401 | T | 1567/389 | 0.340 | 0.324 | 0.93 | 0.39 |
| C04 | rs4697653 | 10337857 | G | 1553/386 | 0.567 | 0.580 | 1.06 | 0.5 |
| C05 | rs728676 | 118087433 | T | 1567/388 | 0.462 | 0.461 | 1.00 | 0.96 |
| C06 | rs6905466 | 19958854 | T | 1567/388 | 0.338 | 0.335 | 0.99 | 0.88 |
| C10 | rs11199158 | 85297471 | G | 1566/388 | 0.645 | 0.621 | 0.90 | 0.22 |
| C01 | rs4845552 | 151746622 | G | 1567/388 | 0.125 | 0.117 | 0.93 | 0.55 |
| C02 | rs1395930 | 123468570 | T | 1567/388 | 0.539 | 0.514 | 0.91 | 0.22 |
| C04 | rs6534683 | 129601758 | T | 1567/388 | 0.652 | 0.635 | 0.93 | 0.38 |
| C02 | rs6747236 | 35901725 | T | 1566/389 | 0.755 | 0.754 | 0.99 | 0.96 |
| C06 | rs3016536 | 162157939 | G | 1567/388 | 0.596 | 0.573 | 0.91 | 0.26 |
| C10 | rs12269612 | 15475877 | T | 1567/389 | 0.376 | 0.362 | 0.94 | 0.49 |
| C17 | rs854793 | 17971249 | A | 1567/388 | 0.232 | 0.237 | 1.03 | 0.75 |
| C12 | rs3742049 | 119438873 | C | 1565/388 | 0.885 | 0.879 | 0.94 | 0.64 |
| C01 | rs2991515 | 57656898 | T | 1565/389 | 0.458 | 0.428 | 0.88 | 0.13 |
| C18 | rs4271662 | 26051291 | C | 1567/388 | 0.595 | 0.601 | 1.02 | 0.78 |
| C06 | rs7739802 | 162154323 | T | 1567/388 | 0.549 | 0.531 | 0.93 | 0.35 |
| C08 | rs10504537 | 73402234 | A | 1567/388 | 0.866 | 0.865 | 0.99 | 0.94 |
| C14 | rs17776453 | 100774793 | G | 1567/388 | 0.956 | 0.954 | 0.95 | 0.81 |
| C10 | rs7086224 | 30857283 | A | 1567/389 | 0.418 | 0.450 | 1.14 | 0.11 |
| C01 | rs12724604 | 20948045 | A | 1566/389 | 0.044 | 0.037 | 0.83 | 0.37 |
| C03 | rs9874646 | 62437858 | T | 1567/388 | 0.669 | 0.673 | 1.01 | 0.86 |
| C05 | rs950195 | 97466690 | G | 1565/389 | 0.654 | 0.657 | 1.01 | 0.9 |
| C14 | rs2281519 | 93846385 | T | 1564/388 | 0.267 | 0.254 | 0.93 | 0.45 |
| C10 | rs4880711 | 5217871 | C | 1562/388 | 0.349 | 0.343 | 0.97 | 0.76 |
| C08 | rs3931282 | 129179963 | T | 1567/389 | 0.517 | 0.528 | 1.05 | 0.57 |
| C10 | rs11198993 | 85244450 | A | 1566/388 | 0.786 | 0.764 | 0.88 | 0.2 |
| C12 | rs1568383 | 88017285 | T | 1567/388 | 0.117 | 0.125 | 1.08 | 0.54 |
| C06 | rs9380681 | 37638433 | C | 1567/388 | 0.315 | 0.325 | 1.04 | 0.61 |
| C02 | rs6709904 | 43933828 | A | 1561/388 | 0.884 | 0.871 | 0.88 | 0.31 |
| C10 | rs2279434 | 45275070 | T | 1567/389 | 0.075 | 0.077 | 1.03 | 0.86 |
| C13 | rs1375719 | 102208783 | C | 1566/388 | 0.492 | 0.492 | 1.00 | 0.98 |
| C10 | rs7921473 | 85156395 | G | 1567/387 | 0.844 | 0.840 | 0.97 | 0.76 |
| C14 | rs1957860 | 53499105 | T | 1567/388 | 0.474 | 0.481 | 1.03 | 0.76 |
| C02 | rs3943703 | 127613790 | C | 1558/385 | 0.228 | 0.232 | 1.02 | 0.81 |
| C08 | rs7006687 | 18277862 | T | 1567/388 | 0.558 | 0.550 | 0.97 | 0.71 |
| C12 | rs1465026 | 65789635 | G | 1564/389 | 0.836 | 0.832 | 0.97 | 0.75 |
| C14 | rs4444235 | 53480669 | T | 1567/388 | 0.546 | 0.523 | 0.91 | 0.26 |
| C02 | rs921083 | 176453128 | G | 1567/387 | 0.820 | 0.796 | 0.86 | 0.12 |
| C08 | rs4732849 | 28249765 | T | 1566/389 | 0.310 | 0.320 | 1.05 | 0.59 |

| | WTCCC (14246/1926) | | | | | Replication (19396/6108) | |
|---|---|---|---|---|---|---|---|
| Chr | nc/na | Ctr. fr | Aff. fr | OR | P | OR (95% CI) | P |
| C06 | 14237/1924 | 0.173 | 0.197 | 1.17 | 0.00026 | 1.15 (1.08-1.22) | 7.33E−06 |
| C20 | 14220/1922 | 0.312 | 0.315 | 1.01 | 0.77 | 1.04 (0.99-1.09) | 0.12 |
| C10 | | | | | | 1.09 (1.02-1.17) | 0.013 |
| C11 | 13841/1882 | 0.482 | 0.488 | 1.02 | 0.53 | 1.03 (0.98-1.09) | 0.2 |
| C07 | 14243/1924 | 0.084 | 0.089 | 1.06 | 0.32 | 1.08 (0.99-1.18) | 0.066 |
| C10 | 14223/1921 | 0.316 | 0.327 | 1.05 | 0.15 | 1.07 (1.00-1.14) | 0.045 |
| C07 | 14241/1925 | 0.096 | 0.099 | 1.03 | 0.62 | 1.05 (0.95-1.17) | 0.31 |
| C01 | 14220/1921 | 0.251 | 0.254 | 1.01 | 0.77 | 1.04 (0.99-1.10) | 0.1 |
| C14 | | | | | | 1.05 (0.98-1.13) | 0.15 |
| C05 | 14239/1924 | 0.201 | 0.217 | 1.10 | 0.022 | 1.08 (1.00-1.17) | 0.045 |
| C12 | 14243/1925 | 0.717 | 0.736 | 1.10 | 0.014 | 1.06 (1.00-1.12) | 0.057 |
| C04 | 14242/1924 | 0.449 | 0.437 | 0.95 | 0.14 | 0.96 (0.91-1.02) | 0.21 |
| C05 | 14243/1925 | 0.182 | 0.196 | 1.09 | 0.046 | 1.05 (0.99-1.13) | 0.11 |
| C05 | 14239/1925 | 0.354 | 0.371 | 1.08 | 0.037 | 1.05 (1.00-1.11) | 0.074 |
| C07 | 14237/1924 | 0.252 | 0.263 | 1.06 | 0.14 | 1.05 (0.98-1.12) | 0.19 |
| C20 | 14212/1921 | 0.397 | 0.407 | 1.04 | 0.21 | 1.04 (0.97-1.10) | 0.28 |
| C17 | 14239/1924 | 0.350 | 0.347 | 0.99 | 0.7 | 1.03 (0.98-1.08) | 0.27 |
| C05 | 14228/1923 | 0.628 | 0.636 | 1.04 | 0.32 | 1.05 (0.98-1.12) | 0.15 |
| C01 | 14219/1922 | 0.560 | 0.560 | 1.00 | 0.97 | 1.02 (0.96-1.08) | 0.54 |
| C05 | 14236/1924 | 0.607 | 0.628 | 1.09 | 0.013 | 1.04 (0.99-1.10) | 0.14 |
| C16 | 14192/1918 | 0.407 | 0.406 | 1.00 | 0.96 | 1.02 (0.96-1.07) | 0.56 |
| C16 | 14243/1925 | 0.406 | 0.406 | 1.00 | 0.99 | 1.02 (0.96-1.07) | 0.58 |
| C13 | 14214/1921 | 0.474 | 0.471 | 0.99 | 0.72 | 1.01 (0.96-1.06) | 0.77 |
| C10 | 14219/1922 | 0.520 | 0.528 | 1.03 | 0.36 | 1.04 (0.98-1.10) | 0.24 |
| C19 | | | | | | 1.00 (0.91-1.11) | 0.94 |
| C06 | 14241/1924 | 0.431 | 0.445 | 1.06 | 0.12 | 1.03 (0.98-1.08) | 0.23 |
| C02 | 14218/1921 | 0.441 | 0.448 | 1.03 | 0.4 | 1.04 (0.98-1.09) | 0.17 |
| C05 | 14228/1919 | 0.106 | 0.116 | 1.10 | 0.069 | 1.07 (0.97-1.18) | 0.19 |
| C17 | 14215/1921 | 0.405 | 0.402 | 0.99 | 0.75 | 1.03 (0.98-1.08) | 0.3 |
| C11 | 14211/1921 | 0.510 | 0.520 | 1.04 | 0.28 | 1.02 (0.96-1.08) | 0.61 |
| C15 | 14222/1922 | 0.074 | 0.076 | 1.04 | 0.54 | 1.02 (0.91-1.14) | 0.69 |
| C17 | 14241/1925 | 0.375 | 0.370 | 0.98 | 0.53 | 1.02 (0.97-1.07) | 0.47 |
| C17 | 14242/1924 | 0.348 | 0.344 | 0.98 | 0.61 | 1.01 (0.96-1.06) | 0.6 |
| C05 | 14233/1923 | 0.178 | 0.187 | 1.06 | 0.18 | 1.04 (0.96-1.13) | 0.29 |
| C17 | 14243/1925 | 0.350 | 0.346 | 0.99 | 0.69 | 1.02 (0.97-1.07) | 0.5 |
| C01 | 14238/1924 | 0.121 | 0.108 | 0.88 | 0.02 | 0.98 (0.91-1.06) | 0.68 |
| C05 | 14242/1925 | 0.945 | 0.946 | 1.03 | 0.68 | 1.04 (0.91-1.19) | 0.56 |
| C08 | 14241/1924 | 0.928 | 0.920 | 0.89 | 0.084 | 0.92 (0.83-1.02) | 0.11 |
| C17 | 14242/1925 | 0.394 | 0.395 | 1.00 | 0.9 | 1.02 (0.97-1.07) | 0.54 |
| C06 | 14241/1925 | 0.157 | 0.154 | 0.97 | 0.55 | 0.97 (0.90-1.05) | 0.5 |
| C17 | 14222/1922 | 0.333 | 0.330 | 0.98 | 0.63 | 1.01 (0.95-1.07) | 0.82 |
| C05 | 14246/1925 | 0.193 | 0.212 | 1.13 | 0.0051 | 1.03 (0.97-1.10) | 0.29 |
| C05 | 14242/1924 | 0.164 | 0.174 | 1.07 | 0.12 | 1.05 (0.97-1.15) | 0.21 |
| C05 | 14240/1924 | 0.290 | 0.301 | 1.06 | 0.13 | 1.06 (0.99-1.13) | 0.11 |
| C14 | 14075/1906 | 0.153 | 0.162 | 1.07 | 0.18 | 1.04 (0.97-1.12) | 0.28 |
| C09 | | | | | | 0.96 (0.87-1.05) | 0.34 |
| C02 | 14239/1925 | 0.656 | 0.667 | 1.05 | 0.19 | 1.02 (0.97-1.07) | 0.53 |
| C01 | 14236/1925 | 0.903 | 0.907 | 1.05 | 0.38 | 1.05 (0.94-1.16) | 0.38 |
| C05 | 14240/1925 | 0.483 | 0.491 | 1.03 | 0.33 | 1.02 (0.96-1.08) | 0.49 |
| C08 | 14242/1925 | 0.623 | 0.625 | 1.01 | 0.83 | 1.01 (0.95-1.08) | 0.72 |
| C15 | 14237/1925 | 0.058 | 0.059 | 1.02 | 0.78 | 1.00 (0.88-1.14) | 0.96 |
| C16 | 14224/1922 | 0.637 | 0.638 | 1.00 | 0.91 | 1.00 (0.94-1.07) | 0.92 |
| C08 | 14241/1925 | 0.623 | 0.625 | 1.01 | 0.83 | 1.01 (0.95-1.08) | 0.72 |
| C21 | 14236/1924 | 0.230 | 0.231 | 1.00 | 0.97 | 0.97 (0.90-1.05) | 0.48 |
| C04 | 14242/1924 | 0.506 | 0.505 | 0.99 | 0.84 | 1.00 (0.94-1.05) | 0.87 |
| C17 | 14240/1925 | 0.310 | 0.301 | 0.96 | 0.24 | 0.99 (0.94-1.04) | 0.64 |
| C12 | 14235/1924 | 0.356 | 0.358 | 1.01 | 0.84 | 1.01 (0.96-1.07) | 0.63 |
| C05 | 14232/1924 | 0.301 | 0.314 | 1.07 | 0.084 | 1.01 (0.96-1.06) | 0.68 |
| C18 | 13709/1832 | 0.188 | 0.192 | 1.03 | 0.52 | 1.02 (0.94-1.10) | 0.68 |
| C13 | 14241/1925 | 0.176 | 0.177 | 1.01 | 0.79 | 1.02 (0.94-1.10) | 0.69 |
| C12 | 14234/1924 | 0.137 | 0.131 | 0.95 | 0.34 | 0.98 (0.91-1.05) | 0.53 |
| C05 | 14240/1924 | 0.317 | 0.319 | 1.01 | 0.82 | 1.02 (0.97-1.07) | 0.42 |
| C04 | 14229/1923 | 0.540 | 0.533 | 0.97 | 0.4 | 1.00 (0.95-1.05) | 0.91 |
| C05 | 14223/1923 | 0.484 | 0.471 | 0.95 | 0.14 | 0.98 (0.93-1.03) | 0.45 |
| C06 | 14224/1922 | 0.331 | 0.326 | 0.98 | 0.55 | 0.98 (0.92-1.05) | 0.55 |
| C10 | 14246/1925 | 0.650 | 0.650 | 1.00 | 0.99 | 0.99 (0.94-1.05) | 0.78 |
| C01 | 14237/1924 | 0.139 | 0.135 | 0.97 | 0.51 | 0.96 (0.90-1.04) | 0.33 |
| C02 | 14242/1925 | 0.498 | 0.495 | 0.99 | 0.71 | 0.98 (0.93-1.02) | 0.29 |
| C04 | 14230/1923 | 0.671 | 0.674 | 1.02 | 0.68 | 1.00 (0.94-1.07) | 0.98 |
| C02 | 14241/1924 | 0.762 | 0.754 | 0.96 | 0.29 | 0.99 (0.93-1.05) | 0.71 |
| C06 | 14219/1921 | 0.628 | 0.628 | 1.00 | 0.99 | 0.99 (0.93-1.05) | 0.67 |
| C10 | 14240/1925 | 0.373 | 0.390 | 1.08 | 0.035 | 1.00 (0.95-1.05) | 0.98 |
| C17 | 14222/1922 | 0.260 | 0.250 | 0.95 | 0.2 | 0.98 (0.93-1.04) | 0.53 |
| C12 | 14239/1924 | 0.897 | 0.898 | 1.01 | 0.88 | 1.00 (0.91-1.09) | 0.96 |
| C01 | 14219/1921 | 0.399 | 0.392 | 0.97 | 0.38 | 0.96 (0.91-1.02) | 0.2 |
| C18 | 14226/1922 | 0.597 | 0.599 | 1.01 | 0.81 | 1.01 (0.96-1.06) | 0.65 |
| C06 | 14219/1922 | 0.624 | 0.620 | 0.98 | 0.64 | 0.97 (0.91-1.04) | 0.42 |
| C08 | 14233/1923 | 0.871 | 0.868 | 0.98 | 0.68 | 0.98 (0.90-1.08) | 0.69 |
| C14 | 14231/1923 | 0.923 | 0.923 | 1.00 | 0.97 | 0.99 (0.88-1.12) | 0.91 |
| C10 | 14217/1921 | 0.450 | 0.441 | 0.96 | 0.26 | 0.99 (0.93-1.05) | 0.68 |
| C01 | 14244/1925 | 0.041 | 0.040 | 0.96 | 0.66 | 0.94 (0.80-1.10) | 0.45 |
| C03 | 14223/1921 | 0.670 | 0.669 | 0.99 | 0.84 | 1.01 (0.96-1.07) | 0.72 |
| C05 | 14236/1924 | 0.649 | 0.647 | 0.99 | 0.82 | 0.97 (0.92-1.02) | 0.24 |
| C14 | 14234/1923 | 0.274 | 0.268 | 0.97 | 0.41 | 0.96 (0.90-1.03) | 0.29 |
| C10 | 14241/1925 | 0.377 | 0.380 | 1.01 | 0.73 | 1.01 (0.94-1.07) | 0.84 |
| C08 | 14236/1924 | 0.533 | 0.536 | 1.01 | 0.74 | 1.04 (0.98-1.09) | 0.19 |
| C10 | 14241/1924 | 0.780 | 0.772 | 0.95 | 0.23 | 0.95 (0.89-1.01) | 0.095 |
| C12 | 14238/1924 | 0.111 | 0.104 | 0.92 | 0.14 | 0.93 (0.85-1.02) | 0.11 |
| C06 | 14229/1923 | 0.365 | 0.362 | 0.99 | 0.73 | 1.00 (0.93-1.06) | 0.91 |
| C02 | 14233/1924 | 0.900 | 0.896 | 0.96 | 0.47 | 0.95 (0.87-1.04) | 0.24 |
| C10 | 14239/1924 | 0.085 | 0.081 | 0.95 | 0.42 | 0.93 (0.85-1.03) | 0.19 |
| C13 | 14222/1922 | 0.510 | 0.510 | 1.00 | 0.95 | 0.98 (0.93-1.04) | 0.49 |
| C10 | 14241/1923 | 0.857 | 0.845 | 0.91 | 0.05 | 0.93 (0.87-1.00) | 0.036 |
| C14 | 14238/1923 | 0.473 | 0.482 | 1.03 | 0.32 | 1.03 (0.98-1.09) | 0.19 |
| C02 | 14171/1913 | 0.233 | 0.222 | 0.94 | 0.14 | 0.95 (0.89-1.03) | 0.21 |
| C08 | 14221/1922 | 0.556 | 0.550 | 0.97 | 0.44 | 0.94 (0.90-0.99) | 0.024 |
| C12 | 14240/1925 | 0.836 | 0.829 | 0.95 | 0.3 | 0.95 (0.89-1.02) | 0.17 |
| C14 | 14232/1923 | 0.537 | 0.536 | 0.99 | 0.86 | 0.97 (0.92-1.02) | 0.18 |
| C02 | 14215/1922 | 0.731 | 0.717 | 0.93 | 0.061 | 0.95 (0.90-1.01) | 0.089 |
| C08 | 14240/1926 | 0.328 | 0.315 | 0.94 | 0.11 | 0.97 (0.92-1.02) | 0.21 |

| Table 4G - MI | | | | Iceland (28321/2395) | | | | | Combined (47717/7577) | |
|---|---|---|---|---|---|---|---|---|---|---|
| Chr | SNP | Pos_B36 | Allele | nc/na | Ctr. fr | Aff. fr | OR | P | OR (95% CI) | P |
| C06 | rs11751605 | 160883220 | C | 28085/2387 | 0.111 | 0.128 | 1.18 | 0.00098 | 1.16 (1.10-1.22) | 1.11E−07 |
| C10 | rs2243547 | 91000459 | G | 28033/2386 | 0.331 | 0.363 | 1.15 | 2.71E−05 | 1.11 (1.06-1.16) | 1.81E−05 |
| C10 | rs1412444 | 90992907 | A | 28293/2394 | 0.361 | 0.389 | 1.13 | 0.00037 | 1.11 (1.06-1.17) | 3.30E−05 |
| C01 | rs12134779 | 63168930 | T | 28320/2394 | 0.292 | 0.325 | 1.17 | 9.41E−06 | 1.09 (1.04-1.14) | 8.32E−05 |
| C11 | rs2163612 | 82856844 | G | 28294/2393 | 0.479 | 0.519 | 1.17 | 1.01E−06 | 1.08 (1.04-1.13) | 0.00011 |
| C05 | rs254850 | 158599309 | T | 28316/2395 | 0.220 | 0.242 | 1.13 | 0.0011 | 1.11 (1.05-1.18) | 0.00013 |
| C17 | rs9902941 | 17674485 | C | 28319/2395 | 0.347 | 0.384 | 1.17 | 2.90E−06 | 1.08 (1.04-1.13) | 0.00023 |
| C20 | rs6076623 | 4084671 | T | 28302/2395 | 0.367 | 0.399 | 1.15 | 4.51E−05 | 1.08 (1.03-1.12) | 0.0004 |
| C05 | rs4921437 | 158623529 | T | 28259/2386 | 0.216 | 0.240 | 1.14 | 0.00049 | 1.10 (1.04-1.15) | 0.00041 |
| C05 | rs832540 | 56234959 | C | 28307/2395 | 0.422 | 0.444 | 1.09 | 0.0061 | 1.08 (1.03-1.12) | 0.00063 |
| C19 | rs12459084 | 17031885 | T | 27962/2387 | 0.350 | 0.380 | 1.14 | 0.00013 | 1.10 (1.04-1.17) | 0.00071 |
| C07 | rs1029396 | 116812056 | G | 28315/2395 | 0.071 | 0.084 | 1.21 | 0.0018 | 1.13 (1.05-1.21) | 0.00076 |
| C17 | rs6502622 | 17775416 | G | 28303/2395 | 0.358 | 0.393 | 1.16 | 6.50E−06 | 1.07 (1.03-1.11) | 0.00078 |
| C17 | rs854787 | 17975744 | C | 28104/2381 | 0.400 | 0.432 | 1.14 | 4.64E−05 | 1.07 (1.03-1.11) | 0.0008 |
| C14 | rs7158073 | 99211442 | T | 28102/2379 | 0.430 | 0.458 | 1.12 | 0.0005 | 1.09 (1.04-1.14) | 0.00083 |
| C10 | rs2243548 | 91000293 | A | 28294/2392 | 0.512 | 0.540 | 1.12 | 0.0007 | 1.08 (1.03-1.13) | 0.0009 |
| C17 | rs4925119 | 17683629 | A | 28218/2387 | 0.345 | 0.380 | 1.16 | 7.74E−06 | 1.07 (1.03-1.11) | 0.0011 |
| C17 | rs3183702 | 17688014 | T | 28108/2388 | 0.348 | 0.384 | 1.17 | 4.13E−06 | 1.07 (1.03-1.11) | 0.0012 |
| C17 | rs854813 | 17944570 | C | 28182/2393 | 0.385 | 0.420 | 1.16 | 1.06E−05 | 1.07 (1.03-1.11) | 0.0013 |
| C05 | rs953861 | 158705160 | G | 28318/2395 | 0.161 | 0.180 | 1.14 | 0.0027 | 1.10 (1.04-1.17) | 0.0014 |
| C07 | rs12534186 | 117079572 | T | 28310/2394 | 0.081 | 0.096 | 1.22 | 0.00056 | 1.13 (1.05-1.22) | 0.0017 |
| C12 | rs2417821 | 20081535 | T | 28317/2395 | 0.754 | 0.771 | 1.10 | 0.014 | 1.07 (1.03-1.13) | 0.0023 |
| C20 | rs1741318 | 4105600 | A | 28315/2394 | 0.450 | 0.474 | 1.10 | 0.0025 | 1.07 (1.03-1.12) | 0.0024 |
| C06 | rs2074464 | 29516507 | T | 28296/2392 | 0.446 | 0.471 | 1.11 | 0.0015 | 1.06 (1.02-1.10) | 0.0031 |
| C07 | rs324594 | 136305863 | C | 28310/2395 | 0.266 | 0.289 | 1.12 | 0.0023 | 1.08 (1.03-1.14) | 0.0033 |
| C05 | rs270654 | 158497687 | G | 28318/2395 | 0.137 | 0.151 | 1.12 | 0.012 | 1.11 (1.03-1.18) | 0.0036 |
| C15 | rs4779984 | 30302218 | G | 28236/2392 | 0.100 | 0.116 | 1.18 | 0.0015 | 1.12 (1.04-1.21) | 0.0038 |
| C05 | rs7709212 | 158696755 | T | 28305/2394 | 0.680 | 0.702 | 1.11 | 0.004 | 1.07 (1.02-1.13) | 0.0039 |
| C05 | rs10045431 | 158747111 | A | 28238/2364 | 0.266 | 0.285 | 1.10 | 0.011 | 1.08 (1.02-1.13) | 0.0039 |
| C17 | rs2955355 | 17889200 | C | 28312/2395 | 0.285 | 0.321 | 1.19 | 8.68E−07 | 1.06 (1.02-1.11) | 0.005 |
| C11 | rs7944761 | 1361414 | T | 28309/2395 | 0.532 | 0.555 | 1.09 | 0.0053 | 1.07 (1.02-1.12) | 0.0057 |
| C05 | rs4704400 | 76586766 | T | 28306/2394 | 0.498 | 0.520 | 1.09 | 0.0057 | 1.06 (1.02-1.11) | 0.006 |
| C17 | rs4925114 | 17651995 | A | 28266/2391 | 0.357 | 0.389 | 1.15 | 4.55E−05 | 1.06 (1.02-1.11) | 0.006 |
| C05 | rs1433048 | 158688423 | G | 27899/2344 | 0.193 | 0.211 | 1.11 | 0.0074 | 1.08 (1.02-1.15) | 0.0069 |
| C05 | rs7447732 | 158461713 | C | 28280/2393 | 0.320 | 0.343 | 1.11 | 0.0033 | 1.06 (1.01-1.10) | 0.0098 |
| C02 | rs2244871 | 128115310 | C | 28251/2394 | 0.413 | 0.433 | 1.08 | 0.013 | 1.06 (1.01-1.10) | 0.011 |
| C01 | rs2946534 | 18647370 | T | 28302/2394 | 0.571 | 0.599 | 1.12 | 0.00034 | 1.06 (1.01-1.11) | 0.012 |
| C04 | rs7661204 | 45728007 | T | 28320/2395 | 0.449 | 0.427 | 0.91 | 0.0055 | 0.95 (0.91-0.99) | 0.012 |
| C05 | rs6556861 | 94554256 | G | 28317/2395 | 0.570 | 0.591 | 1.09 | 0.0082 | 1.06 (1.01-1.10) | 0.012 |
| C06 | rs7753765 | 71732495 | A | 28312/2395 | 0.144 | 0.166 | 1.18 | 0.00014 | 1.08 (1.01-1.14) | 0.014 |
| C05 | rs870347 | 6898035 | T | 28313/2395 | 0.926 | 0.937 | 1.20 | 0.0052 | 1.12 (1.02-1.24) | 0.015 |
| C10 | rs12269612 | 15475877 | T | 28314/2395 | 0.401 | 0.423 | 1.09 | 0.0078 | 1.05 (1.01-1.10) | 0.015 |
| C15 | rs2110209 | 24946433 | A | 28318/2395 | 0.048 | 0.061 | 1.28 | 0.00051 | 1.12 (1.02-1.23) | 0.018 |
| C02 | rs12329252 | 212093968 | T | 28321/2395 | 0.681 | 0.703 | 1.10 | 0.0047 | 1.05 (1.01-1.09) | 0.019 |
| C14 | rs8003722 | 68388015 | A | 28317/2394 | 0.106 | 0.119 | 1.14 | 0.0085 | 1.07 (1.01-1.14) | 0.019 |
| C21 | rs2828495 | 24038081 | A | 28316/2395 | 0.212 | 0.234 | 1.14 | 0.001 | 1.07 (1.01-1.12) | 0.021 |
| C05 | rs270661 | 158492732 | A | 28319/2394 | 0.210 | 0.227 | 1.10 | 0.011 | 1.06 (1.01-1.12) | 0.022 |
| C08 | rs334198 | 18933089 | C | 28309/2393 | 0.922 | 0.913 | 0.89 | 0.037 | 0.91 (0.84-0.99) | 0.022 |
| C01 | rs2476601 | 114179091 | A | 28313/2395 | 0.129 | 0.152 | 1.21 | 3.14E−05 | 1.07 (1.01-1.14) | 0.024 |
| C13 | rs4769613 | 28036609 | T | 28313/2394 | 0.465 | 0.494 | 1.12 | 0.00041 | 1.05 (1.01-1.09) | 0.024 |
| C08 | rs3102526 | 96858538 | G | 27995/2382 | 0.647 | 0.666 | 1.09 | 0.013 | 1.05 (1.01-1.10) | 0.029 |
| C08 | rs3134517 | 96859237 | G | 28308/2395 | 0.647 | 0.666 | 1.09 | 0.016 | 1.05 (1.00-1.10) | 0.032 |
| C01 | rs3748744 | 19828833 | G | 28280/2394 | 0.898 | 0.907 | 1.11 | 0.063 | 1.09 (1.01-1.17) | 0.034 |
| C04 | rs4282162 | 21426819 | T | 28305/2395 | 0.530 | 0.555 | 1.11 | 0.002 | 1.05 (1.00-1.09) | 0.036 |
| C16 | rs8050136 | 52373776 | A | 28316/2394 | 0.404 | 0.426 | 1.10 | 0.0046 | 1.05 (1.00-1.09) | 0.039 |
| C16 | rs3751812 | 52375961 | T | 28246/2392 | 0.403 | 0.426 | 1.10 | 0.0041 | 1.05 (1.00-1.09) | 0.039 |
| C17 | rs854793 | 17971249 | A | 28313/2395 | 0.202 | 0.229 | 1.18 | 3.33E−05 | 1.05 (1.00-1.10) | 0.041 |
| C10 | rs4880711 | 5217871 | C | 27953/2379 | 0.351 | 0.367 | 1.07 | 0.042 | 1.05 (1.00-1.10) | 0.043 |
| C12 | rs12304836 | 124654446 | C | 28205/2385 | 0.143 | 0.163 | 1.17 | 0.00054 | 1.06 (1.00-1.12) | 0.043 |
| C05 | rs38055 | 52596401 | T | 28311/2395 | 0.387 | 0.403 | 1.07 | 0.052 | 1.04 (1.00-1.08) | 0.053 |
| C09 | rs2297538 | 138684489 | G | 28226/2395 | 0.730 | 0.753 | 1.13 | 0.0012 | 1.06 (1.00-1.13) | 0.053 |
| C16 | rs1870843 | 81316815 | T | 28313/2395 | 0.602 | 0.623 | 1.09 | 0.0081 | 1.05 (1.00-1.10) | 0.064 |
| C06 | rs6905466 | 19958854 | T | 28315/2395 | 0.332 | 0.355 | 1.11 | 0.0027 | 1.04 (1.00-1.10) | 0.074 |
| C12 | rs11045689 | 21092930 | G | 28210/2386 | 0.301 | 0.319 | 1.09 | 0.017 | 1.04 (1.00-1.08) | 0.083 |
| C13 | rs1887263 | 22381716 | A | 28175/2393 | 0.204 | 0.216 | 1.08 | 0.067 | 1.05 (0.99-1.11) | 0.089 |
| C18 | rs4271662 | 26051291 | C | 28256/2390 | 0.587 | 0.602 | 1.06 | 0.061 | 1.03 (0.99-1.07) | 0.11 |
| C18 | rs1365254 | 70238283 | T | 28319/2395 | 0.196 | 0.208 | 1.08 | 0.066 | 1.05 (0.99-1.11) | 0.11 |
| C06 | rs3016536 | 162157939 | G | 28320/2395 | 0.519 | 0.538 | 1.08 | 0.02 | 1.04 (0.99-1.08) | 0.12 |
| C12 | rs3742049 | 119438873 | C | 28300/2394 | 0.919 | 0.928 | 1.14 | 0.029 | 1.06 (0.98-1.14) | 0.13 |
| C03 | rs9874646 | 62437858 | T | 28314/2394 | 0.717 | 0.729 | 1.06 | 0.11 | 1.03 (0.99-1.08) | 0.17 |
| C14 | rs2281519 | 93846385 | T | 28281/2395 | 0.260 | 0.282 | 1.12 | 0.0023 | 1.04 (0.98-1.09) | 0.17 |
| C02 | rs6747236 | 35901725 | T | 28311/2395 | 0.717 | 0.739 | 1.12 | 0.0021 | 1.03 (0.98-1.08) | 0.18 |
| C04 | rs6534683 | 129601758 | T | 28310/2395 | 0.656 | 0.670 | 1.06 | 0.076 | 1.03 (0.99-1.08) | 0.18 |
| C01 | rs2991515 | 57656898 | T | 28319/2395 | 0.483 | 0.507 | 1.10 | 0.0033 | 1.03 (0.99-1.07) | 0.19 |
| C04 | rs4697653 | 10337857 | G | 28161/2392 | 0.524 | 0.549 | 1.11 | 0.0021 | 1.03 (0.99-1.07) | 0.19 |
| C06 | rs9380681 | 37638433 | C | 28318/2395 | 0.311 | 0.327 | 1.08 | 0.038 | 1.03 (0.98-1.08) | 0.19 |
| C10 | rs11199158 | 85297471 | G | 28319/2395 | 0.618 | 0.635 | 1.08 | 0.024 | 1.03 (0.99-1.08) | 0.19 |
| C13 | rs1375719 | 102208783 | C | 28316/2394 | 0.499 | 0.517 | 1.07 | 0.032 | 1.03 (0.99-1.08) | 0.19 |
| C08 | rs3931282 | 129179963 | T | 28319/2395 | 0.568 | 0.543 | 0.90 | 0.0016 | 0.97 (0.93-1.02) | 0.21 |
| C14 | rs17776453 | 100774793 | G | 28313/2394 | 0.955 | 0.961 | 1.15 | 0.095 | 1.06 (0.96-1.17) | 0.24 |
| C01 | rs12724604 | 20948045 | A | 28248/2386 | 0.037 | 0.045 | 1.23 | 0.0098 | 1.07 (0.95-1.20) | 0.25 |
| C05 | rs950195 | 97466690 | G | 28228/2395 | 0.633 | 0.656 | 1.10 | 0.0033 | 1.03 (0.98-1.07) | 0.25 |
| C05 | rs728676 | 118087433 | T | 28310/2394 | 0.444 | 0.468 | 1.10 | 0.0023 | 1.02 (0.98-1.07) | 0.25 |
| C06 | rs7739802 | 162154323 | T | 28266/2393 | 0.493 | 0.511 | 1.07 | 0.027 | 1.03 (0.98-1.08) | 0.26 |
| C10 | rs7086224 | 30857283 | A | 28287/2393 | 0.419 | 0.437 | 1.08 | 0.028 | 1.03 (0.98-1.07) | 0.27 |
| C01 | rs4845552 | 151746622 | G | 28269/2393 | 0.128 | 0.145 | 1.15 | 0.0025 | 1.03 (0.97-1.09) | 0.3 |
| C08 | rs10504537 | 73402234 | A | 28213/2387 | 0.881 | 0.891 | 1.11 | 0.035 | 1.04 (0.97-1.11) | 0.32 |
| C02 | rs1395930 | 123468570 | T | 28303/2393 | 0.499 | 0.523 | 1.10 | 0.0032 | 1.02 (0.98-1.06) | 0.33 |
| C02 | rs3943703 | 127613790 | C | 28280/2395 | 0.214 | 0.229 | 1.09 | 0.02 | 1.02 (0.97-1.08) | 0.44 |
| C10 | rs2279434 | 45275070 | T | 28318/2395 | 0.067 | 0.080 | 1.20 | 0.0029 | 1.03 (0.95-1.12) | 0.46 |
| C10 | rs11198993 | 85244450 | A | 28239/2387 | 0.697 | 0.714 | 1.08 | 0.022 | 1.02 (0.97-1.07) | 0.46 |
| C10 | rs7921473 | 85156395 | G | 28314/2395 | 0.820 | 0.840 | 1.15 | 0.0011 | 1.02 (0.96-1.08) | 0.51 |
| C12 | rs1465026 | 65789635 | G | 28309/2395 | 0.837 | 0.852 | 1.12 | 0.011 | 1.02 (0.96-1.08) | 0.52 |
| C12 | rs1568383 | 88017285 | T | 28316/2395 | 0.098 | 0.111 | 1.15 | 0.008 | 1.02 (0.95-1.09) | 0.55 |
| C08 | rs4732849 | 28249765 | T | 28321/2395 | 0.275 | 0.295 | 1.10 | 0.0069 | 1.01 (0.97-1.06) | 0.56 |
| C08 | rs7006687 | 18277862 | T | 28306/2392 | 0.579 | 0.604 | 1.11 | 0.0018 | 1.01 (0.97-1.05) | 0.73 |
| C02 | rs921083 | 176453128 | G | 28310/2394 | 0.781 | 0.798 | 1.11 | 0.0092 | 1.01 (0.96-1.06) | 0.78 |
| C02 | rs6709904 | 43933828 | A | 28315/2395 | 0.909 | 0.918 | 1.12 | 0.058 | 1.01 (0.94-1.08) | 0.84 |
| C14 | rs1957860 | 53499105 | T | 28313/2395 | 0.448 | 0.439 | 0.96 | 0.27 | 1.00 (0.96-1.04) | 0.86 |
| C14 | rs4444235 | 53480669 | T | 28305/2394 | 0.536 | 0.543 | 1.03 | 0.37 | 1.00 (0.96-1.04) | 0.88 |
| Table 4H - CAD | | | | | | | | | | |
| | | Pos in | | Iceland (28321/3047) | | | | | Combined (47717/9155) | |
| Chr | SNP | Build 36 | Allele | nc/na | Ctr. fr | Aff. fr | OR | P | OR (95% CI) | P |
| C06 | rs11751605 | 160883220 | C | 28085/3033 | 0.111 | 0.128 | 1.17 | 0.00039 | 1.16 (1.10-1.22) | 1.16E−08 |
| C20 | rs6076623 | 4084671 | T | 28302/3047 | 0.367 | 0.400 | 1.15 | 3.56E−06 | 1.08 (1.04-1.13) | 0.0000292 |
| C10 | rs1412444 | 90992907 | A | 28293/3045 | 0.361 | 0.383 | 1.10 | 0.0014 | 1.10 (1.05-1.15) | 5.21E−05 |
| C11 | rs2163612 | 82856844 | G | 28294/3045 | 0.479 | 0.513 | 1.15 | 3.61E−06 | 1.08 (1.04-1.12) | 0.0000533 |
| C07 | rs1029396 | 116812056 | G | 28315/3047 | 0.071 | 0.086 | 1.24 | 0.0000612 | 1.14 (1.07-1.22) | 0.0000824 |
| C10 | rs2243547 | 91000459 | G | 28033/3033 | 0.331 | 0.355 | 1.11 | 6.60E−04 | 1.09 (1.04-1.14) | 1.10E−04 |
| C07 | rs12534186 | 117079572 | T | 28310/3046 | 0.081 | 0.098 | 1.24 | 0.0000302 | 1.15 (1.07-1.23) | 0.00021 |
| C01 | rs12134779 | 63168930 | T | 28320/3046 | 0.292 | 0.316 | 1.12 | 2.80E−04 | 1.07 (1.03-1.12) | 3.70E−04 |
| C14 | rs7158073 | 99211442 | T | 28102/3027 | 0.430 | 0.453 | 1.10 | 0.0016 | 1.08 (1.03-1.13) | 0.00076 |
| C05 | rs254850 | 158599309 | T | 28316/3047 | 0.220 | 0.236 | 1.10 | 0.0082 | 1.09 (1.04-1.15) | 0.00095 |
| C12 | rs2417821 | 20081535 | T | 28317/3047 | 0.754 | 0.772 | 1.10 | 0.0042 | 1.08 (1.03-1.12) | 0.001 |
| C04 | rs7661204 | 45728007 | T | 28320/3047 | 0.449 | 0.425 | 0.91 | 0.00074 | 0.94 (0.90-0.97) | 0.0011 |
| C05 | rs4921437 | 158623529 | T | 28259/3037 | 0.216 | 0.235 | 1.11 | 0.0022 | 1.08 (1.03-1.13) | 0.0011 |
| C05 | rs832540 | 56234959 | C | 28307/3047 | 0.422 | 0.443 | 1.09 | 0.0043 | 1.07 (1.03-1.11) | 0.0012 |
| C07 | rs324594 | 136305863 | C | 28310/3047 | 0.266 | 0.287 | 1.11 | 0.0018 | 1.08 (1.03-1.13) | 0.0014 |
| C20 | rs1741318 | 4105600 | A | 28315/3046 | 0.450 | 0.473 | 1.10 | 0.0013 | 1.07 (1.03-1.12) | 0.0019 |
| C17 | rs9902941 | 17674485 | C | 28319/3047 | 0.347 | 0.371 | 1.11 | 9.50E−04 | 1.06 (1.02-1.10) | 0.0028 |
| C05 | rs7709212 | 158696755 | T | 28305/3046 | 0.680 | 0.699 | 1.09 | 0.0061 | 1.07 (1.02-1.12) | 0.0029 |
| C01 | rs2946534 | 18647370 | T | 28302/3046 | 0.571 | 0.596 | 1.11 | 0.00045 | 1.07 (1.02-1.11) | 0.0032 |
| C05 | rs6556861 | 94554256 | G | 28317/3047 | 0.570 | 0.590 | 1.08 | 0.006 | 1.06 (1.02-1.10) | 0.0034 |
| C16 | rs8050136 | 52373776 | A | 28316/3046 | 0.404 | 0.430 | 1.11 | 0.00029 | 1.06 (1.02-1.10) | 0.0038 |
| C16 | rs3751812 | 52375961 | T | 28246/3044 | 0.403 | 0.430 | 1.12 | 0.00024 | 1.06 (1.02-1.10) | 0.004 |
| C13 | rs4769613 | 28036609 | T | 28313/3046 | 0.465 | 0.493 | 1.12 | 0.00013 | 1.05 (1.02-1.10) | 0.0059 |
| C10 | rs2243548 | 91000293 | A | 28294/3043 | 0.512 | 0.532 | 1.08 | 0.0087 | 1.06 (1.02-1.11) | 0.0064 |
| C19 | rs12459084 | 17031885 | T | 27962/3037 | 0.350 | 0.372 | 1.10 | 0.002 | 1.07 (1.02-1.13) | 0.0067 |
| C06 | rs2074464 | 29516507 | T | 28296/3044 | 0.446 | 0.466 | 1.08 | 0.006 | 1.05 (1.01-1.09) | 0.0076 |
| C02 | rs2244871 | 128115310 | C | 28251/3046 | 0.413 | 0.430 | 1.08 | 0.015 | 1.05 (1.01-1.10) | 0.0079 |
| C05 | rs270654 | 158497687 | G | 28318/3047 | 0.137 | 0.149 | 1.10 | 0.021 | 1.09 (1.02-1.16) | 0.0092 |
| C17 | rs854787 | 17975744 | C | 28104/3028 | 0.400 | 0.420 | 1.09 | 4.60E−03 | 1.05 (1.01-1.09) | 0.0097 |
| C11 | rs7944761 | 1361414 | T | 28309/3047 | 0.532 | 0.554 | 1.09 | 0.0027 | 1.06 (1.01-1.10) | 0.011 |
| C15 | rs4779984 | 30302218 | G | 28236/3043 | 0.100 | 0.114 | 1.15 | 0.003 | 1.10 (1.02-1.18) | 0.011 |
| C17 | rs6502622 | 17775416 | G | 28303/3047 | 0.358 | 0.380 | 1.10 | 1.90E−03 | 1.05 (1.01-1.09) | 0.013 |
| C17 | rs3183702 | 17688014 | T | 28108/3038 | 0.348 | 0.371 | 1.10 | 1.30E−03 | 1.05 (1.01-1.09) | 0.014 |
| C05 | rs1433048 | 158688423 | G | 27899/2989 | 0.193 | 0.207 | 1.09 | 0.021 | 1.07 (1.01-1.13) | 0.015 |
| C17 | rs4925119 | 17683629 | A | 28218/3036 | 0.345 | 0.367 | 1.10 | 2.10E−03 | 1.05 (1.01-1.09) | 0.015 |
| C01 | rs2476601 | 114179091 | A | 28313/3047 | 0.129 | 0.148 | 1.18 | 9.72E−05 | 1.07 (1.01-1.14) | 0.016 |
| C05 | rs870347 | 6898035 | T | 28313/3047 | 0.926 | 0.936 | 1.17 | 0.0088 | 1.11 (1.02-1.21) | 0.017 |
| C08 | rs334198 | 18933089 | C | 28309/3045 | 0.922 | 0.915 | 0.91 | 0.082 | 0.92 (0.85-0.98) | 0.018 |
| C17 | rs854813 | 17944570 | C | 28182/3042 | 0.385 | 0.406 | 1.09 | 3.10E−03 | 1.05 (1.01-1.09) | 0.018 |
| C06 | rs7753765 | 71732495 | A | 28312/3047 | 0.144 | 0.165 | 1.18 | 0.0000474 | 1.07 (1.01-1.13) | 0.02 |
| C17 | rs4925114 | 17651995 | A | 28266/3042 | 0.357 | 0.379 | 1.10 | 1.70E−03 | 1.05 (1.01-1.09) | 0.02 |
| C05 | rs270661 | 158492732 | A | 28319/3046 | 0.210 | 0.223 | 1.08 | 0.025 | 1.06 (1.01-1.11) | 0.022 |
| C05 | rs953861 | 158705160 | G | 28318/3047 | 0.161 | 0.172 | 1.08 | 0.052 | 1.07 (1.01-1.13) | 0.022 |
| C05 | rs10045431 | 158747111 | A | 28238/3014 | 0.266 | 0.277 | 1.06 | 0.095 | 1.06 (1.01-1.11) | 0.022 |
| C14 | rs8003722 | 68388015 | A | 28317/3046 | 0.106 | 0.116 | 1.11 | 0.024 | 1.07 (1.01-1.13) | 0.024 |
| C09 | rs2297538 | 138684489 | G | 28226/3047 | 0.730 | 0.752 | 1.12 | 0.00079 | 1.06 (1.01-1.12) | 0.027 |
| C02 | rs12329252 | 212093968 | T | 28321/3047 | 0.681 | 0.700 | 1.09 | 0.0059 | 1.04 (1.00-1.08) | 0.029 |
| C01 | rs3748744 | 19828833 | G | 28280/3046 | 0.898 | 0.908 | 1.11 | 0.033 | 1.08 (1.01-1.16) | 0.031 |
| C05 | rs4704400 | 76586766 | T | 28306/3046 | 0.498 | 0.514 | 1.07 | 0.023 | 1.04 (1.00-1.09) | 0.036 |
| C08 | rs3102526 | 96858538 | G | 27995/3019 | 0.647 | 0.664 | 1.08 | 0.015 | 1.05 (1.00-1.09) | 0.046 |
| C15 | rs2110209 | 24946433 | A | 28318/3047 | 0.048 | 0.057 | 1.19 | 0.0061 | 1.10 (1.00-1.20) | 0.048 |
| C16 | rs1870843 | 81316815 | T | 28313/3047 | 0.602 | 0.621 | 1.08 | 0.0098 | 1.04 (1.00-1.09) | 0.048 |
| C08 | rs3134517 | 96859237 | G | 28308/3047 | 0.647 | 0.664 | 1.08 | 0.018 | 1.05 (1.00-1.09) | 0.049 |
| C21 | rs2828495 | 24038081 | A | 28316/3047 | 0.212 | 0.232 | 1.12 | 0.00089 | 1.05 (1.00-1.11) | 0.051 |
| C04 | rs4282162 | 21426819 | T | 28305/3047 | 0.530 | 0.552 | 1.09 | 0.0031 | 1.04 (1.00-1.08) | 0.052 |
| C17 | rs2955355 | 17889200 | C | 28312/3047 | 0.285 | 0.309 | 1.12 | 3.30E−04 | 1.04 (1.00-1.08) | 0.052 |
| C12 | rs11045689 | 21092930 | G | 28210/3037 | 0.301 | 0.317 | 1.08 | 0.015 | 1.04 (1.00-1.08) | 0.054 |
| C05 | rs7447732 | 158461713 | C | 28280/3045 | 0.320 | 0.337 | 1.08 | 0.015 | 1.04 (1.00-1.08) | 0.064 |
| C18 | rs1365254 | 70238283 | T | 28319/3047 | 0.196 | 0.209 | 1.08 | 0.036 | 1.05 (1.00-1.11) | 0.065 |
| C13 | rs1887263 | 22381716 | A | 28175/3042 | 0.204 | 0.217 | 1.08 | 0.037 | 1.05 (1.00-1.11) | 0.066 |
| C12 | rs12304836 | 124654446 | C | 28205/3034 | 0.143 | 0.160 | 1.14 | 0.00095 | 1.05 (0.99-1.11) | 0.084 |
| C05 | rs38055 | 52596401 | T | 28311/3047 | 0.387 | 0.399 | 1.05 | 0.091 | 1.03 (0.99-1.07) | 0.091 |
| C04 | rs4697653 | 10337857 | G | 28161/3040 | 0.524 | 0.543 | 1.08 | 0.0081 | 1.03 (0.99-1.07) | 0.1 |
| C05 | rs728676 | 118087433 | T | 28310/3046 | 0.444 | 0.468 | 1.10 | 0.001 | 1.03 (0.99-1.07) | 0.1 |
| C06 | rs6905466 | 19958854 | T | 28315/3047 | 0.332 | 0.351 | 1.09 | 0.0058 | 1.04 (0.99-1.09) | 0.1 |
| C10 | rs11199158 | 85297471 | G | 28319/3047 | 0.618 | 0.637 | 1.09 | 0.0062 | 1.03 (0.99-1.08) | 0.1 |
| C01 | rs4845552 | 151746622 | G | 28269/3044 | 0.128 | 0.146 | 1.16 | 0.00041 | 1.05 (0.99-1.11) | 0.11 |
| C02 | rs1395930 | 123468570 | T | 28303/3045 | 0.499 | 0.528 | 1.12 | 0.00012 | 1.03 (0.99-1.07) | 0.12 |
| C04 | rs6534683 | 129601758 | T | 28310/3047 | 0.656 | 0.671 | 1.07 | 0.034 | 1.04 (0.99-1.08) | 0.12 |
| C02 | rs6747236 | 35901725 | T | 28311/3047 | 0.717 | 0.734 | 1.09 | 0.0098 | 1.03 (0.99-1.08) | 0.13 |
| C06 | rs3016536 | 162157939 | G | 28320/3047 | 0.519 | 0.537 | 1.08 | 0.013 | 1.03 (0.99-1.08) | 0.13 |
| C10 | rs12269612 | 15475877 | T | 28314/3047 | 0.401 | 0.418 | 1.07 | 0.024 | 1.03 (0.99-1.07) | 0.13 |
| C17 | rs854793 | 17971249 | A | 28313/3047 | 0.202 | 0.220 | 1.12 | 1.80E−03 | 1.03 (0.99-1.08) | 0.14 |
| C12 | rs3742049 | 119438873 | C | 28300/3046 | 0.919 | 0.928 | 1.13 | 0.023 | 1.05 (0.98-1.13) | 0.15 |
| C01 | rs2991515 | 57656898 | T | 28319/3047 | 0.483 | 0.508 | 1.10 | 0.00077 | 1.03 (0.99-1.07) | 0.16 |
| C18 | rs4271662 | 26051291 | C | 28256/3039 | 0.587 | 0.599 | 1.05 | 0.092 | 1.03 (0.99-1.06) | 0.16 |
| C06 | rs7739802 | 162154323 | T | 28266/3043 | 0.493 | 0.512 | 1.08 | 0.011 | 1.03 (0.99-1.07) | 0.18 |
| C08 | rs10504537 | 73402234 | A | 28213/3035 | 0.881 | 0.891 | 1.11 | 0.027 | 1.04 (0.98-1.11) | 0.2 |
| C14 | rs17776453 | 100774793 | G | 28313/3045 | 0.955 | 0.962 | 1.17 | 0.033 | 1.06 (0.97-1.16) | 0.21 |
| C10 | rs7086224 | 30857283 | A | 28287/3045 | 0.419 | 0.434 | 1.06 | 0.04 | 1.03 (0.98-1.07) | 0.22 |
| C01 | rs12724604 | 20948045 | A | 28248/3037 | 0.037 | 0.043 | 1.19 | 0.021 | 1.07 (0.96-1.19) | 0.23 |
| C03 | rs9874646 | 62437858 | T | 28314/3046 | 0.717 | 0.726 | 1.05 | 0.16 | 1.02 (0.98-1.07) | 0.24 |
| C05 | rs950195 | 97466690 | G | 28228/3046 | 0.633 | 0.654 | 1.10 | 0.002 | 1.02 (0.98-1.07) | 0.25 |
| C14 | rs2281519 | 93846385 | T | 28281/3045 | 0.260 | 0.276 | 1.08 | 0.014 | 1.03 (0.98-1.08) | 0.27 |
| C10 | rs4880711 | 5217871 | C | 27953/3015 | 0.351 | 0.359 | 1.04 | 0.26 | 1.02 (0.98-1.07) | 0.34 |
| C08 | rs3931282 | 129179963 | T | 28319/3047 | 0.568 | 0.548 | 0.92 | 0.007 | 0.98 (0.94-1.02) | 0.39 |
| C10 | rs11198993 | 85244450 | A | 28239/3038 | 0.697 | 0.717 | 1.10 | 0.0037 | 1.02 (0.97-1.06) | 0.43 |
| C12 | rs1568383 | 88017285 | T | 28316/3045 | 0.098 | 0.110 | 1.14 | 0.0049 | 1.03 (0.96-1.09) | 0.45 |
| C06 | rs9380681 | 37638433 | C | 28318/3047 | 0.311 | 0.319 | 1.04 | 0.25 | 1.02 (0.97-1.06) | 0.46 |
| C02 | rs6709904 | 43933828 | A | 28315/3046 | 0.909 | 0.919 | 1.14 | 0.014 | 1.03 (0.96-1.10) | 0.47 |
| C10 | rs2279434 | 45275070 | T | 28318/3047 | 0.067 | 0.076 | 1.15 | 0.014 | 1.03 (0.95-1.11) | 0.49 |
| C13 | rs1375719 | 102208783 | C | 28316/3046 | 0.499 | 0.511 | 1.05 | 0.12 | 1.01 (0.97-1.06) | 0.53 |
| C10 | rs7921473 | 85156395 | G | 28314/3047 | 0.820 | 0.838 | 1.13 | 0.0013 | 1.02 (0.96-1.07) | 0.55 |
| C14 | rs1957860 | 53499105 | T | 28313/3047 | 0.448 | 0.444 | 0.98 | 0.55 | 1.01 (0.97-1.05) | 0.55 |
| C02 | rs3943703 | 127613790 | C | 28280/3046 | 0.214 | 0.225 | 1.07 | 0.06 | 1.01 (0.96-1.07) | 0.6 |
| C08 | rs7006687 | 18277862 | T | 28306/3044 | 0.579 | 0.604 | 1.11 | 0.00068 | 1.01 (0.97-1.05) | 0.6 |
| C12 | rs1465026 | 65789635 | G | 28309/3047 | 0.837 | 0.849 | 1.10 | 0.024 | 1.01 (0.96-1.06) | 0.67 |
| C14 | rs4444235 | 53480669 | T | 28305/3046 | 0.536 | 0.543 | 1.03 | 0.32 | 0.99 (0.96-1.03) | 0.7 |
| C02 | rs921083 | 176453128 | G | 28310/3044 | 0.781 | 0.794 | 1.08 | 0.04 | 1.00 (0.96-1.05) | 0.98 |
| C08 | rs4732849 | 28249765 | T | 28321/3047 | 0.275 | 0.285 | 1.05 | 0.13 | 1.00 (0.96-1.04) | 0.99 |

TABLE 5
Surrogate markers for marker rs11751605. The markers were
selected from the Caucasian data set of the HapMap sample (CEU;
see http://www.hapmap.org), using a cutoff of r² of 0.2. The search
was done in a 2Mb interval flanking the rs11751605 marker.
The table shows (1) chromosome; (2) SNP rs name; (3) position in
NCBI build 36; (4) value for D′ to rs11751605; (5) value for r² to
rs11751605; (6) P-value of the association with rs11751605.
Surrogates of rs11751605 based on HapMap CEU v22

| | | Pos in | | | |
|---|---|---|---|---|---|
| Chr | SNP | Build 36 | D′ | R2 | P |
| C06 | rs3798156 | 160596206 | 0.55 | 0.22 | 5.8E−05 |
| C06 | rs3127573 | 160601383 | 0.80 | 0.43 | 4.9E−08 |
| C06 | rs3127572 | 160602208 | 0.71 | 0.36 | 4.7E−07 |
| C06 | rs3119309 | 160605062 | 0.80 | 0.44 | 2.4E−08 |
| C06 | rs7756836 | 160609247 | 0.80 | 0.44 | 2.4E−08 |
| C06 | rs7757336 | 160609548 | 0.56 | 0.29 | 3.6E−06 |
| C06 | rs3119310 | 160612365 | 0.80 | 0.44 | 2.4E−08 |
| C06 | rs3119311 | 160613097 | 0.80 | 0.44 | 2.4E−08 |
| C06 | rs3127578 | 160618145 | 0.80 | 0.44 | 2.4E−08 |
| C06 | rs3127586 | 160624902 | 0.85 | 0.67 | 9.9E−13 |
| C06 | rs12206585 | 160633514 | 0.92 | 0.72 | 1.1E−13 |
| C06 | rs12203303 | 160633686 | 0.83 | 0.55 | 4.6E−09 |
| C06 | rs2183470 | 160635083 | 0.85 | 0.68 | 1.5E−13 |
| C06 | rs3120151 | 160637462 | 0.85 | 0.68 | 1.5E−13 |
| C06 | rs3103350 | 160642995 | 0.85 | 0.68 | 1.5E−13 |
| C06 | rs3127583 | 160647121 | 0.85 | 0.68 | 1.5E−13 |
| C06 | rs3106172 | 160647965 | 0.85 | 0.64 | 1.2E−10 |
| C06 | rs3106170 | 160649914 | 0.86 | 0.72 | 6.6E−13 |
| C06 | rs3125057 | 160651662 | 0.84 | 0.60 | 2.7E−11 |
| C06 | rs3125056 | 160655271 | 0.87 | 0.45 | 4.8E−06 |
| C06 | rs3125055 | 160656777 | 0.86 | 0.37 | 2.2E−06 |
| C06 | rs3106167 | 160657380 | 0.85 | 0.68 | 1.5E−13 |
| C06 | rs3127591 | 160657792 | 0.92 | 0.71 | 3.2E−12 |
| C06 | rs3125052 | 160658363 | 0.85 | 0.68 | 1.5E−13 |
| C06 | rs3120140 | 160658821 | 0.90 | 0.58 | 1.1E−08 |
| C06 | rs3103349 | 160660711 | 0.85 | 0.68 | 1.5E−13 |
| C06 | rs3125050 | 160660826 | 0.85 | 0.68 | 1.5E−13 |
| C06 | rs3120139 | 160661612 | 0.85 | 0.68 | 1.5E−13 |
| C06 | rs3125049 | 160674702 | 0.86 | 0.74 | 4.0E−15 |
| C06 | rs12209517 | 160676726 | 0.72 | 0.46 | 2.4E−07 |
| C06 | rs3120137 | 160691182 | 0.93 | 0.87 | 1.2E−17 |
| C06 | rs3127574 | 160711360 | 1.00 | 0.20 | 3.1E−08 |
| C06 | rs6905958 | 160717615 | 1.00 | 0.20 | 3.1E−08 |
| C06 | rs3123633 | 160719595 | 1.00 | 0.20 | 3.1E−08 |
| C06 | rs3123634 | 160722229 | 1.00 | 0.20 | 3.1E−08 |
| C06 | rs1317652 | 160727151 | 1.00 | 0.20 | 2.9E−08 |
| C06 | rs884742 | 160727422 | 1.00 | 0.20 | 3.1E−08 |
| C06 | rs7740824 | 160728891 | 0.91 | 0.45 | 1.1E−09 |
| C06 | rs3105748 | 160730710 | 1.00 | 0.20 | 3.1E−08 |
| C06 | rs3105749 | 160740968 | 1.00 | 0.20 | 3.1E−08 |
| C06 | rs3105752 | 160742915 | 1.00 | 0.20 | 2.9E−08 |
| C06 | rs10945670 | 160747660 | 1.00 | 0.20 | 3.1E−08 |
| C06 | rs10806731 | 160747825 | 1.00 | 0.20 | 3.1E−08 |
| C06 | rs9364554 | 160753654 | 0.92 | 0.46 | 5.2E−11 |
| C06 | rs12194182 | 160754505 | 0.92 | 0.46 | 5.2E−11 |
| C06 | rs3106166 | 160758351 | 1.00 | 0.22 | 2.2E−08 |
| C06 | rs10455782 | 160759340 | 1.00 | 0.39 | 4.6E−12 |
| C06 | rs7758229 | 160760242 | 1.00 | 0.38 | 2.2E−11 |
| C06 | rs3123636 | 160762527 | 1.00 | 0.37 | 1.9E−11 |
| C06 | rs3106164 | 160770263 | 1.00 | 0.37 | 6.9E−10 |
| C06 | rs388170 | 160773276 | 1.00 | 0.36 | 1.0E−11 |
| C06 | rs9365164 | 160776833 | 0.91 | 0.33 | 3.9E−08 |
| C06 | rs2292334 | 160778178 | 1.00 | 0.33 | 2.8E−10 |
| C06 | rs2048327 | 160783522 | 1.00 | 0.33 | 4.5E−11 |
| C06 | rs7769879 | 160785635 | 1.00 | 0.33 | 4.5E−11 |
| C06 | rs3918285 | 160788644 | 1.00 | 0.33 | 4.5E−11 |
| C06 | rs3918286 | 160788658 | 1.00 | 0.33 | 1.1E−10 |
| C06 | rs1810126 | 160792141 | 1.00 | 0.34 | 3.0E−11 |
| C06 | rs3088442 | 160792642 | 1.00 | 0.33 | 4.5E−11 |
| C06 | rs2063347 | 160802019 | 1.00 | 0.33 | 4.5E−11 |
| C06 | rs3106162 | 160803284 | 1.00 | 0.33 | 4.5E−11 |
| C06 | rs2063346 | 160804265 | 1.00 | 0.34 | 3.5E−11 |
| C06 | rs1112073 | 160810832 | 1.00 | 0.38 | 8.0E−12 |
| C06 | rs3127602 | 160812231 | 1.00 | 0.37 | 8.9E−12 |
| C06 | rs3123629 | 160826076 | 1.00 | 0.37 | 7.6E−12 |
| C06 | rs3127599 | 160827124 | 1.00 | 0.37 | 7.6E−12 |
| C06 | rs3127569 | 160837549 | 1.00 | 0.39 | 4.0E−12 |
| C06 | rs3127596 | 160873025 | 1.00 | 0.38 | 4.7E−12 |
| C06 | rs3124785 | 160874495 | 1.00 | 0.38 | 7.6E−12 |
| C06 | rs11751605 | 160883220 | 1.00 | 1.00 | 0.0E+00 |
| C06 | rs10455872 | 160930108 | 1.00 | 0.55 | 1.3E−11 |
| C06 | rs2315065 | 161028134 | 0.66 | 0.35 | 4.6E−07 |
| C06 | rs9458266 | 161798149 | 0.58 | 0.22 | 1.7E−04 |

TABLE 6
Key to the sequence listing provided herein.

| Seq ID No: | SNP |
|---|---|
| 1 | rs11751605 |
| 2 | rs6076623 |
| 3 | rs1412444 |
| 4 | rs2163612 |
| 5 | rs1029396 |
| 6 | rs2243547 |
| 7 | rs12534186 |
| 8 | rs12134779 |
| 9 | rs7158073 |
| 10 | rs254850 |
| 11 | rs2417821 |
| 12 | rs7661204 |
| 13 | rs4921437 |
| 14 | rs832540 |
| 15 | rs324594 |
| 16 | rs1741318 |
| 17 | rs9902941 |
| 18 | rs7709212 |
| 19 | rs2946534 |
| 20 | rs6556861 |
| 21 | rs8050136 |
| 22 | rs3751812 |
| 23 | rs4769613 |
| 24 | rs2243548 |
| 25 | rs12459084 |
| 26 | rs2074464 |
| 27 | rs2244871 |
| 28 | rs270654 |
| 29 | rs854787 |
| 30 | rs7944761 |
| 31 | rs4779984 |
| 32 | rs6502622 |
| 33 | rs3183702 |
| 34 | rs1433048 |
| 35 | rs4925119 |
| 36 | rs2476601 |
| 37 | rs870347 |
| 38 | rs334198 |
| 39 | rs854813 |
| 40 | rs7753765 |
| 41 | rs4925114 |
| 42 | rs270661 |
| 43 | rs953861 |
| 44 | rs10045431 |
| 45 | rs8003722 |
| 46 | rs2297538 |
| 47 | rs12329252 |
| 48 | rs3748744 |
| 49 | rs4704400 |
| 50 | rs3102526 |
| 51 | rs2110209 |
| 52 | rs1870843 |
| 53 | rs3134517 |
| 54 | rs2828495 |
| 55 | rs4282162 |
| 56 | rs2955355 |
| 57 | rs11045689 |
| 58 | rs7447732 |
| 59 | rs1365254 |
| 60 | rs1887263 |
| 61 | rs12304836 |
| 62 | rs38055 |
| 63 | rs4697653 |
| 64 | rs728676 |
| 65 | rs6905466 |
| 66 | rs11199158 |
| 67 | rs4845552 |
| 68 | rs1395930 |
| 69 | rs6534683 |
| 70 | rs6747236 |
| 71 | rs3016536 |
| 72 | rs12269612 |
| 73 | rs854793 |
| 74 | rs3742049 |
| 75 | rs2991515 |
| 76 | rs4271662 |
| 77 | rs7739802 |
| 78 | rs10504537 |
| 79 | rs17776453 |
| 80 | rs7086224 |
| 81 | rs12724604 |
| 82 | rs9874646 |
| 83 | rs950195 |
| 84 | rs2281519 |
| 85 | rs4880711 |
| 86 | rs3931282 |
| 87 | rs11198993 |
| 88 | rs1568383 |
| 89 | rs9380681 |
| 90 | rs6709904 |
| 91 | rs2279434 |
| 92 | rs1375719 |
| 93 | rs7921473 |
| 94 | rs1957860 |
| 95 | rs3943703 |
| 96 | rs7006687 |
| 97 | rs1465026 |
| 98 | rs4444235 |
| 99 | rs921083 |
| 100 | rs4732849 |
| 101 | rs3798156 |
| 102 | rs3127573 |
| 103 | rs3127572 |
| 104 | rs3119309 |
| 105 | rs7756836 |
| 106 | rs7757336 |
| 107 | rs3119310 |
| 108 | rs3119311 |
| 109 | rs3127578 |
| 110 | rs3127586 |
| 111 | rs12206585 |
| 112 | rs12203303 |
| 113 | rs2183470 |
| 114 | rs3120151 |
| 115 | rs3103350 |
| 116 | rs3127583 |
| 117 | rs3106172 |
| 118 | rs3106170 |
| 119 | rs3125057 |
| 120 | rs3125056 |
| 121 | rs3125055 |
| 122 | rs3106167 |
| 123 | rs3127591 |
| 124 | rs3125052 |
| 125 | rs3120140 |
| 126 | rs3103349 |
| 127 | rs3125050 |
| 128 | rs3120139 |
| 129 | rs3125049 |
| 130 | rs12209517 |
| 131 | rs3120137 |
| 132 | rs3127574 |
| 133 | rs6905958 |
| 134 | rs3123633 |
| 135 | rs3123634 |
| 136 | rs1317652 |
| 137 | rs884742 |
| 138 | rs7740824 |
| 139 | rs3105748 |
| 140 | rs3105749 |
| 141 | rs3105752 |
| 142 | rs10945670 |
| 143 | rs10806731 |
| 144 | rs9364554 |
| 145 | rs12194182 |
| 146 | rs3106166 |
| 147 | rs10455782 |
| 148 | rs7758229 |
| 149 | rs3123636 |
| 150 | rs3106164 |
| 151 | rs388170 |
| 152 | rs9365164 |
| 153 | rs2292334 |
| 154 | rs2048327 |
| 155 | rs7769879 |
| 156 | rs3918285 |
| 157 | rs3918286 |
| 158 | rs1810126 |
| 159 | rs3088442 |
| 160 | rs2063347 |
| 161 | rs3106162 |
| 162 | rs2063346 |
| 163 | rs1112073 |
| 164 | rs3127602 |
| 165 | rs3123629 |
| 166 | rs3127599 |
| 167 | rs3127569 |
| 168 | rs3127596 |
| 169 | rs3124785 |
| 170 | rs10455872 |
| 171 | rs2315065 |
| 172 | rs9458266 |

1. A method for determining a susceptibility to myocardial infarction or coronary artery disease in a human individual, comprising determining the presence or absence of at least one allele of at least one polymorphic marker in a nucleic acid sample obtained from the individual or in a genotype dataset derived from the individual, wherein the at least one polymorphic marker is selected from the markers set forth in Table 4, and markers in linkage disequilibrium therewith, and wherein the presence of the at least one allele is indicative of a susceptibility to cardiovascular disease.

2. The method according to claim 1, wherein the at least one polymorphic marker is selected from rs11751605 (SEQ ID NO:1), rs6076623 (SEQ ID NO:2), rs1412444 (SEQ ID NO:3), rs2163612 (SEQ ID NO:4), rs1029396 (SEQ ID NO:5), rs2243547 (SEQ ID NO:6), rs12534186 (SEQ ID NO:7), rs12134779 (SEQ ID NO:8), rs7158073 (SEQ ID NO:9), rs254850 (SEQ ID NO:10), rs2417821 (SEQ ID NO:11), rs7661204 (SEQ ID NO:12), rs4921437 (SEQ ID NO:13), rs832540 (SEQ ID NO:14), rs324594 (SEQ ID NO:15), rs1741318 (SEQ ID NO:16), rs9902941 (SEQ ID NO:17), rs7709212 (SEQ ID NO:18), rs2946534 (SEQ ID NO:19), rs6556861 (SEQ ID NO:20), rs8050136 (SEQ ID NO:21), rs3751812 (SEQ ID NO:22), rs4769613 (SEQ ID NO:23), rs2243548 (SEQ ID NO:24), rs12459084 (SEQ ID NO:25), rs2074464 (SEQ ID NO:26), rs2244871 (SEQ ID NO:27), rs270654 (SEQ ID NO:28), rs854787 (SEQ ID NO:29), rs7944761 (SEQ ID NO:30), rs4779984 (SEQ ID NO:31), rs6502622 (SEQ ID NO:32), rs3183702 (SEQ ID NO:33), rs1433048 (SEQ ID NO:34), rs4925119 (SEQ ID NO:35), rs2476601 (SEQ ID NO:36), rs870347 (SEQ ID NO:37), rs334198 (SEQ ID NO:38), rs854813 (SEQ ID NO:39), rs7753765 (SEQ ID NO:40), rs4925114 (SEQ ID NO:41), rs270661 (SEQ ID NO:42), rs953861 (SEQ ID NO:43), rs10045431 (SEQ ID NO:44), rs8003722 (SEQ ID NO:45), rs2297538 (SEQ ID NO:46), rs12329252 (SEQ ID NO:47), rs3748744 (SEQ ID NO:48), rs4704400 (SEQ ID NO:49), rs3102526 (SEQ ID NO:50), rs2110209 (SEQ ID NO:51), rs1870843 (SEQ ID NO:52) and rs3134517 (SEQ ID NO:53), and markers in linkage disequilibrium therewith.

3. The method according to claim 2, wherein the at least one polymorphic marker is selected from rs11751605 (SEQ ID NO:1), rs6076623 (SEQ ID NO:2), rs1412444 (SEQ ID NO:3), rs2163612 (SEQ ID NO:4), rs1029396 (SEQ ID NO:5), rs2243547 (SEQ ID NO:6), rs12534186 (SEQ ID NO:7), rs12134779 (SEQ ID NO:8) and rs7158073 (SEQ ID NO:9), and markers in linkage disequilibrium therewith.

4. The method according to any of the preceding claims, wherein the at least one polymorphic marker is selected from rs11751605 (SEQ ID NO:1), rs6076623 (SEQ ID NO:2), rs1412444 (SEQ ID NO:3), rs2163612 (SEQ ID NO:4) and rs1029396 (SEQ ID NO:5), and markers in linkage disequilibrium therewith.

5. The method according to any of the preceding claims, wherein the at least one polymorphic marker is selected from rs11751605 (SEQ ID NO:1), and markers in linkage disequilibrium therewith.

6. The method according to any of the preceding claims, further comprising assessing the frequency of at least one haplotype in the individual.

7. The method of any of the preceding claims, wherein the susceptibility conferred by the presence of the at least one allele or haplotype is increased susceptibility.

8. The method according to claim 7, wherein the presence of allele C in rs11751605, is indicative of increased susceptibility to myocardial infarction or coronary artery disease.

9. The method according to claim 7 or 8, wherein the presence of the at least one allele or haplotype is indicative of increased susceptibility with a relative risk (RR) or odds ratio (OR) of at least 1.15.

10. The method according to any of the claims 1-6, wherein the susceptibility conferred by the presence of the at least one allele or haplotype is decreased susceptibility.

11. A method of identification of a marker for use in assessing susceptibility to coronary artery disease or myocardial infarction, the method comprising
a. identifying at least one polymorphic marker in linkage disequilibrium with at least one of the markers set forth in Table 4;
b. determining the genotype status of a sample of individuals diagnosed with, or having a susceptibility to, coronary artery disease or myocardial infarction; and
c. determining the genotype status of a sample of control individuals;
wherein a significant difference in frequency of at least one allele in at least one polymorphism in individuals diagnosed with, or having a susceptibility to, coronary artery disease or myocardial infarction, as compared with the frequency of the at least one allele in the control sample is indicative of the at least one polymorphism being useful for assessing susceptibility to coronary artery disease or myocardial infarction.

12. The method according to claim 11, wherein an increase in frequency of the at least one allele in the at least one polymorphism in individuals diagnosed with, or having a susceptibility to, coronary artery disease or myocardial infarction, as compared with the frequency of the at least one allele in the control sample is indicative of the at least one polymorphism being useful for assessing increased susceptibility to coronary artery disease or myocardial infarction.

13. The method according to claim 11 or claim 12, wherein a decrease in frequency of the at least one allele in the at least one polymorphism in individuals diagnosed with, or having a susceptibility to, coronary artery disease or myocardial infarction, as compared with the frequency of the at least one allele in the control sample is indicative of the at least one polymorphism being useful for assessing decreased susceptibility to, or protection against, coronary artery disease or myocardial infarction.

14. The method according to any of the preceding claims wherein the presence of the marker or haplotype is indicative of a different response rate of the subject to a particular treatment modality for myocardial infarction or coronary artery disease.

15. The method according to claim 14, wherein the treatment modality is a coronary stenosis method selected from balloon angioplasty, stenting, cutting balloon angioplasty, percutaneous transluminal coronary angioplasty (PTCA), directional coronary atherectomy, rotational coronary atherectomy, brachytherapy, drug-eluting stent (DES) insertion, metal stent insertion, and coronary artery surgery.

16. The method of claim 14, wherein the treatment modality is administration of a therapeutic agent for preventing and/or ameliorating symptoms associated with the Cardiovascular disease.

17. A method of assessing an individual for probability of response to a therapeutic agent for preventing and/or ameliorating symptoms associated with coronary artery disease or myocardial infarction, comprising: determining the presence or absence of at least one allele of at least one polymorphic marker in a nucleic acid sample obtained from the individual, wherein the at least one polymorphic marker is selected from the group consisting of the polymorphic markers set forth in Table 4, and markers in linkage disequilibrium therewith, wherein the presence of the at least one allele of the at least one marker is indicative of a probability of a positive response to the therapeutic agent.

18. A method of predicting prognosis of an individual diagnosed with coronary artery disease or myocardial infarction, the method comprising determining the presence or absence of at least one allele of at least one polymorphic marker in a nucleic acid sample obtained from the individual, wherein the at least one polymorphic marker is selected from the group consisting of the polymorphic markers set forth in Table 4, and markers in linkage disequilibrium therewith, wherein the presence of the at least one allele is indicative of a worse prognosis of the coronary artery disease or myocardial infarction in the individual.

19. A method of monitoring progress of treatment of an individual undergoing treatment for coronary artery disease or myocardial infarction, the method comprising determining the presence or absence of at least one allele of at least one polymorphic marker in a nucleic acid sample obtained from the individual, wherein the at least one polymorphic marker is selected from the markers set forth in Tables 4, and markers in linkage disequilibrium therewith, wherein the presence of the at least one allele is indicative of the treatment outcome of the individual.

20. The method according to any of the claims 11-19, wherein the at least one marker is selected from rs11751605 (SEQ ID NO:1), rs6076623 (SEQ ID NO:2), rs1412444 (SEQ ID NO:3), rs2163612 (SEQ ID NO:4), rs1029396 (SEQ ID NO:5), rs2243547 (SEQ ID NO:6), rs12534186 (SEQ ID NO:7), rs12134779 (SEQ ID NO:8) and rs7158073 (SEQ ID NO:9), and markers in linkage disequilibrium therewith.

21. The method according to claim 20, wherein the at least one marker is rs11751605.

22. The method of any of the preceding claims, further comprising assessing at least one biomarker in a sample from the individual.

23. The method of claim 22, wherein the biomarker is a cardiac marker or an inflammatory marker.

24. The method of claim 22 or claim 23, wherein the at least one biomarker is selected from creatin kinase, troponin, glycogen phosphorylase, C-reactive protein (CRP), serum amyloid A, fibrinogen, interleukin-6, tissue necrosis factor-alpha, soluble vascular cell adhesion molecules (sVCAM), soluble intervascular adhesion molecules (sICAM), E-selectin, matrix metalloprotease type-1, matrix metalloprotease type-2, matrix metalloprotease type-3, matrix metalloprotease type-9, serum sCD40L, leukotrienes, leukotriene metabolites, interleukin-6, tissue necrosis factor-alpha, myeloperoxidase (MPO), and N-tyrosine.

25. The method of claim 24, wherein the leukotriene is selected from LTB4, LTC4, LTD4 and LTE4.

26. The method of any of the preceding claims, further comprising analyzing a sample comprising genomic DNA from the human individual or a genotype dataset derived from the human individual for the presence or absence of at least one at-risk allele of at least one at-risk variant for coronary artery disease or myocardial infarction not in linkage disequilibrium with any one of the markers set forth in Table 4.

27. The method of any of the claims 1-6, comprising determining the presence or absence of at least one allele in at least two polymorphic markers, wherein the presence of the at least one allele in the at least two polymorphic markers is indicative of an increased susceptibility to coronary artery disease or myocardial infarction.

28. The method of any of the preceding claims, further comprising analyzing non-genetic information to make risk assessment, diagnosis, or prognosis of the individual.

29. The method of claim 28, wherein the non-genetic information is selected from age, gender, ethnicity, socioeconomic status, previous disease diagnosis, medical history of subject, family history of coronary artery disease or myocardial infarction, biochemical measurements, and clinical measurements.

30. A kit for assessing susceptibility to coronary artery disease or myocardial infarction in a human individual, the kit comprising reagents for selectively detecting at least one allele of at least one polymorphic marker in the genome of the individual, wherein the polymorphic marker is selected from the markers set forth in Tables 4, and markers in linkage disequilibrium therewith, and wherein the presence of the at least one allele is indicative of a susceptibility to coronary artery disease or myocardial infarction.

31. The kit of claim 30, wherein the at least one polymorphic marker is selected from rs11751605 (SEQ ID NO:1), rs6076623 (SEQ ID NO:2), rs1412444 (SEQ ID NO:3), rs2163612 (SEQ ID NO:4), rs1029396 (SEQ ID NO:5), rs2243547 (SEQ ID NO:6), rs12534186 (SEQ ID NO:7), rs12134779 (SEQ ID NO:8) and rs7158073 (SEQ ID NO:9), and markers in linkage disequilibrium therewith.

32. The method kit according to claim 30 or claim 31, wherein the at least one polymorphic marker is rs11751605 (SEQ ID NO:1).

33. The kit according to any of the claims 30-32, wherein the reagents comprise at least one contiguous oligonucleotide that hybridizes to a fragment of the genome of the individual comprising the at least one polymorphic marker, a buffer and a detectable label.

34. The kit according to any of the claims 30-32, wherein the reagents comprise at least one pair of oligonucleotides that hybridize to opposite strands of a genomic nucleic acid segment obtained from the subject, wherein each oligonucleotide primer pair is designed to selectively amplify a fragment of the genome of the individual that includes one polymorphic marker, and wherein the fragment is at least 30 base pairs in size.

35. The kit according to claim 33 or 34, wherein the at least one oligonucleotide is completely complementary to the genome of the individual.

36. The kit according to any of the claims 33-35, wherein the oligonucleotide is about 18 to about 50 nucleotides in length.

37. The kit according any of the claims 33-36, wherein the oligonucleotide is 20-30 nucleotides in length.

38. The kit according to any of the claims 30-37, wherein the kit comprises:
a. a detection oligonucleotide probe that is from 5-100 nucleotides in length;
b. an enhancer oligonucleotide probe that is from 5-100 nucleotides in length; and
c. an endonuclease enzyme;
wherein the detection oligonucleotide probe specifically hybridizes to a first segment of the nucleic acid whose nucleotide sequence is set forth in any one of SEQ ID NO:1-172 and
wherein the detection oligonucleotide probe comprises a detectable label at its 3′ terminus and a quenching moiety at its 5′ terminus;
wherein the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid;
wherein a single base gap exists between the first segment and the second segment, such that when the oligonucleotide probe and the enhancer oligonucleotide probe are both hybridized to the nucleic acid, a single base gap exists between the oligonucleotides; and
wherein treating the nucleic acid with the endonuclease will cleave the detectable label from the 3′ terminus of the detection probe to release free detectable label when the detection probe is hybridized to the nucleic acid.

39. Use of an oligonucleotide probe in the manufacture of a reagent for diagnosing and/or assessing susceptibility to coronary artery disease or myocardial infarction in a human individual, wherein the probe hybridizes to a segment of a nucleic acid whose nucleotide sequence is set forth in any one of SEQ ID NO:1-172, wherein the probe is 15-500 nucleotides in length.

40. A computer-readable medium on which is stored:
a. an identifier for at least one polymorphic marker;
b. an indicator of the frequency of at least one allele of said at least one polymorphic marker in a plurality of individuals diagnosed with coronary artery disease or myocardial infarction; and
c. an indicator of the frequency of the least one allele of said at least one polymorphic markers in a plurality of reference individuals;
wherein the at least one polymorphic marker is selected from the polymorphic markers set forth in Table 4, and polymorphic markers in linkage disequilibrium therewith.

41. The medium according to claim 40, wherein the polymorphic marker is selected from rs11751605 (SEQ ID NO:1), rs6076623 (SEQ ID NO:2), rs1412444 (SEQ ID NO:3), rs2163612 (SEQ ID NO:4), rs1029396 (SEQ ID NO:5), rs2243547 (SEQ ID NO:6), rs12534186 (SEQ ID NO:7), rs12134779 (SEQ ID NO:8) and rs7158073 (SEQ ID NO:9), and markers in linkage disequilibrium therewith.

42. An apparatus for determining a genetic indicator for coronary artery disease or myocardial infarction in a human individual, comprising:
a computer readable memory; and
a routine stored on the computer readable memory;
wherein the routine is adapted to be executed on a processor to analyze marker and/or haplotype information for at least one human individual with respect to at least one polymorphic marker selected from the markers set forth in Table 4, and markers in linkage disequilibrium therewith, and generate an output based on the marker or haplotype information, wherein the output comprises a risk measure of the at least one marker or haplotype as a genetic indicator of coronary artery disease or myocardial infarction for the human individual.

43. The apparatus of claim 42, wherein the routine further comprises an indicator of the frequency of at least one allele of at least one polymorphic marker or at least one haplotype in a plurality of individuals diagnosed with coronary artery disease or myocardial infarction, and an indicator of the frequency of at the least one allele of at least one polymorphic marker or at least one haplotype in a plurality of reference individuals, and wherein a risk measure is based on a comparison of the at least one marker and/or haplotype status for the human individual to the indicator of the frequency of the at least one marker and/or haplotype information for the plurality of individuals diagnosed with coronary artery disease or myocardial infarction.

44. The apparatus according to claim 42 or 43, wherein the at least one polymorphic marker is selected from rs11751605 (SEQ ID NO:1), rs6076623 (SEQ ID NO:2), rs1412444 (SEQ ID NO:3), rs2163612 (SEQ ID NO:4), rs1029396 (SEQ ID NO:5), rs2243547 (SEQ ID NO:6), rs12534186 (SEQ ID NO:7), rs12134779 (SEQ ID NO:8) and rs7158073 (SEQ ID NO:9), and markers in linkage disequilibrium therewith.

45. The apparatus or medium according to any of the claims 42-44, wherein the at least one polymorphic marker is rs11751605 (SEQ ID NO:1).

46. The apparatus of any of the claims 42-45, wherein the risk measure is characterized by an Odds Ratio (OR) or a Relative Risk (RR).

47. The method, use, medium or apparatus according to any of the preceding claims, wherein linkage disequilibrium between markers is characterized by particular numerical values for the linkage disequilibrium measures r² and/or |D′|.

48. The method according to claim 47, wherein linkage disequilibrium is characterized by a numerical values for r² of greater than 0.2.

49. The method according to claim 47 or claim 48, wherein linkage disequilibrium is characterized by numerical values for r² of greater than 0.5.

50. The method according to claim 47, wherein linkage disequilibrium is characterized by numerical values for r² of greater than 0.2 and/or |D′| of greater than 0.8


SEQUENCE LISTING (172 sequences)

SEQ ID NO: 1 - DNA, 599 bp, Homo sapiens
caactcaacagcagagtgaaatgcgatgcgatcaggtttgtcgatggcatttcacaaacttacagaaggaagtctctttagaggagaacagcaattgagcacaggtgccctggtgtagaaaggagatgaggaagcaaaacatgcaggcagatgggtctgtgtcctatgtgttcttcctaagagacacccaagcaaactgaacttacgaattgcatgttccactgaaacgatacccaaaatgtccaggttctgtgggttcggactgattactcttcatttatggggatcacaacccataayttgttcatgtgtttgtgaatagatgaatagcctcggcattgaattcaatgattcacaaaagtgtaaaaaaaaatctgtgcctgtgatagactgtaggtaagtttgctcttgtcctccctcactgcaaagtgtcacatttctaaaaacttccctttcttttcatctgacagtgagacaatggatatccatagaaaccctcagagcatcttcaatattttccaaaagtattggaacttggtgcaaatcactgtgcctacagattttgatggaactgcccagatcaagaaaaagatttgtat

SEQ ID NO: 2 - DNA, 599 bp, Homo sapiens
ctcaggctctacatggcctggggtgcgtatctgaggacactggtgggttgagtccccccagttgctggaagcggggtagcagtcactacacccaaaagaaggtacagagggcagacacaaagggcaaaggttccactgccctgggcagtgccatgaggggctggcagggtaggggttgactggctctcagtctgagctccccagatatcccacacacacctcctcaaaggagggcaaggccaagccaatcacaccccactttgatccctgcccctgggccaggccgtcctgcgggatggkaggggataacatttctcaaagactgttctgcgcaatctcaagcgttcaggggacaaataagggtagaaactggcaaattcactccactaccctacgggagattcagaatacatttaagagaattaaaggtcctgacaagtcctgtagaaaagaaactgtttagtgtttcccagcctgatctgaccgtggaaatcttttttttaaatgactgccaataatgtcccacccaccaggaaaacacctgctgggaaaagctgatgtggaaagatgctgctcactggaaggccacgaacatggta

SEQ ID NO: 3 - DNA, 599 bp, Homo sapiens
ctaaatctgttcatagcatttggtcctcatgttctttctctattctgagaggccctccctgtaggggatcactgatgtgtatgtcatacctgctatgagctaacctcagtggggagtcttcaaggaagtctacagtgaacaactcagttctctaaaggaattgtgattttcctaagaactgcataggcgctttcctgatggtttgcatttccacattctaaaacagagcatggacagaggctctttactgtccttcactcctattcaagagcaaggttgttgccactggtgttatttccrgtgtttaaaggcaaatagataaaaagaatggagaaggcctttcacacccaaagtgaaacgcgtcctcttctcatgcatcccgcttcccctcctgacttcctcctctattattaatagcaccagcatcctcctaatgacctagcctggaatctgcagaatcattcttgaccacgggtcctctctaggccacccacgttaaattgcttcagttcatctttttcttctgtttccctgaccaccagcctacattaggctcttgttaccacttaactgggcgcctgaaataagctcagcagctg

SEQ ID NO: 4 - DNA, 599 bp, Homo sapiens
tatcaactaaatgaatactggttctagtcaaactgtcctggataattgcacctattttcctctttgttcatgttgttaacttgaaatgttctgactggtgaataatcaaccagcatttatacatgttatgtggctgctgcacttcacagatagaaaacttgaaatctattacctaagtaataacggcttgaaccatcttagtgggtttctcagataagaagacttcaagtattcactggactgtattcattttcatgtgaacattatagatgagattttagcccttattattgatttagkattgttcctatctgcataaagtgccttttttctcttcttgcataaagttattctagcctcagggtaatgaggacatttctctccaggaggtagaatatgatgtggcaaatgtatctgcatatccagaaatgctttataaagcagctgcttgctctgagcactcattgtggtggaagccaagttaaatgaggtgtgctataatgtaatagcatttttatttgttaacaaacaagaaatttacatacctcatgcaagcctttctctcttggcatttaacccgcatttaatttatatatata

SEQ ID NO: 5 - DNA, 599 bp, Homo sapiens
ctggggccagaataagattttattactcttttatagagcttttaaattactataagtgcttcttgtttcatagaagatgaatactctagcaaaataaagttctcattacctggctggaggaaagatacatatttaaacatttaataattgccagatacactataattacacttttaattctaacagaataatacaaaagttctacaaatgataatgtgatatgccacagaaaataagtttaatatgtacagaaggatacctcatgatgtttgtttctttttgcaatctcacttggcatckttccatctttggtttgtagcattttattagctccaagttcaagcaacttcaaaactatatttttatgaccctgacgtgctgcccacgttaaagcctgtaagtagggggaaaagaagattgtttaaagagaaggagtgtatatgagacagacaggaaaagttttcaacagtatttatgacacaataaaaaagagaacatagttaattaatggaatgatgcaatctcttcattttacaatataatgtaaacatatttccaaagttgtaattgcaaagctgactgcaaagctcattgatttt

SEQ ID NO: 6 - DNA, 599 bp, Homo sapiens
atcttttttcagaatctgtactatgcatctaatatatacgtggcactttgcttggtcctgggggtacaaagctgagtcagattgttacaaagtctaaataactgagataaaggtgttaatacagcccagaaaagcacacttaattcagcctggaaggttttttttcccccctccttcgagattttcagagtatctccttcttagtaagaattgcttggcttagtatgttgagaaaaacatactaagtatgtagtgttgtgttggaaggaaaacttaactgggtgagattttcagctgagkagctttagctgttactctcatggaccttcctctgggcggttgatacaggttctgtaggaaaaccgctgaatgaaagcagctcctgcagatgctattctgttttcaattgtggtaattcctttaattttttccctttcaatttactccttattctctagtaataatggacttttgggaggttgcatttatgataaaaaatagcctggggctgtacaatccttcctatcatgaagaagtacaaagttgtaagtagaagcagtggccttgctttgccaatgcacactgaaggggataacgtt

SEQ ID NO: 7 - DNA, 599 bp, Homo sapiens
tattctaaatgttaatatcaaaaatattattttcaattcaccccaggcacagagaactaagtattattattgctattgcaccggcattccccaatgagacagtgattttcttttaagacatttttaaataatataggcagaattaagtagacggtgatctggtaagtagatgtttcagggtaacagctgtgcaatgctccatgcagggaattagattgtcattttattccttaccaggaacatacattcagttaaacaattatttgacttctgctcttccactgatttctaagttgaggytctctcttgtgcctgtctgatcagataagtagagttgtgccttggtttatagatgagataaatgtgtatttgaataagcataagttaaagaaattttaaaatcccttaggaagctaggcttatcagagaaatccaaggaaatacattaacaaactaggaatttgttctaacaggttaattataactcataaacttattgggtttttttaccttttaattttatattacatttgcttataataaggaatattgctaggaataaaattttttaatattctacaattaacaattatctcaat

SEQ ID NO: 8 - DNA, 599 bp, Homo sapiens
atcctgaacaactgaatagaagtcagggcctaccttgaaccagtcagaaaatgcattccagaggagatgcttttgaatcacacgtacaagtcccagaaaacttctctacaacctttacctttgtcctttaggtggaacaaagtactatataaaactgtgtggggattattttgatgagggttgaaaaatgaaggctcttatccgttatttaaattcctgtgtgcaccgcccccttcacaaagtgtgggagtaggtcttctacatcaagatgcctggagtttggttcagtgaaaacctttyggagggcctggggatcttgattgtccctcagtgccctgatgaaatgtttttatttatcctgcttgctgcctggagttgacactggtaatatcaatcattccctgccagcgtattaacatggctgcacctgcttaaaccatccacatgttgtaactcacaagaagttgggaatcaaatgcagtattatttatgtactcccttaacttgtcccatactacaaaaatttccactctgacaccatctgctagggaaagggatttatctatcgatgtgaaacacccactgaaccggtatcacat

SEQ ID NO: 9 - DNA, 599 bp, Homo sapiens
cctgggtccctctgacggcatactcttcctctgcgcagagttcatcccgaagacacggagcaccccgcggcctacagcgcgggcgcccacgcgggcgccccgccgagggcgccccacggccgctccccccgcgccaaccccgcggccagcgcggcccacccagcagccagggagccggaggggcggcgggcggcggcgggggcggctgaactcggcgagccgggcgttccgggatggcgaggtgcgcctggtgcggcccgcgggcctgagctctggcagcgggcgcgtggaggtgttcgygggcggacgctggggcaccgtgtgcgacgactcctggaacatcagcggcgccgccgtcgtgtgtcgccagctggggtttgcctacgccgtgcgcgccgtcaagagagccgagttcggccagggcggctcgctgcccattctgctggacgatgtgcgctgcgcgggctgggagcggaacctgctggagtgccagcacaacggcgtgggcacccacaactgcgagcacgacgaggatgcgggcgtcgtgtgcagccaccagaaccccgacctgtaggcaacacgccgctgccccaggccat

SEQ ID NO: 10 - DNA, 599 bp, Homo sapiens
gaggtgagaggatcatttgagcacaggagttcaaagctgcagtgagctaagaaggtgccactgcatgggtgatagagcaagaccccatctcttataaacaaacaaacaaacaattttttttatcccctagttctttaatagatcatttatgttttcttttctcctgtcctttccccttttttcctattctttcttcctccttcccgaggtatggaaggattgtgaatctactcagtcaatcttaaaaggggcaatggggagagggaaaacaaggaaccctcacaggctaatttttaaacyacatctcttaagaaaatatggagaagcaaagagaggaaaatgtaaattacctgaaattctaccactgtaaatatgttgatatactttcagactttttcctatgcatttataacacacacacacacacacacatatataagttgcaaaaaatggccaacagtatatatatacatactcttttgtagtcccctttaagcaacttattgtgaatgtctttccctctcaagctttatactgacaacatcattttgaaaagctacatgactttccattgtgtgaatgtactatcatttatgaac

SEQ ID NO: 11 - DNA, 599 bp, Homo sapiens
ctgtggacaaatattgattgtggcacacataaagacagttttgtaagtgtgagtggtgagaaaacattttaaaatatcaatcgtccagagaagattagaaaatttgcacactttatgaatggttccaataacccgcagattgtctcatattagacttcctcctgagctgatctccagtcatataagagtgcatattgctatcaccgcttgacaaactggtgtaaaaattaaggaaacataacttttccttgtagcatcttaagaataacaagttgtccttgacagtaataaagaaataaygcagggaattcagaagaaaactatttcaagggagtcatagaataaaagccagagtaatgagtttggtgacaacaacactgagacaagagaaagaatagaaactgctttaaaaaatttgcaaaatctactgatcgcagtgatccttctcttttttgttgacatacttaagaataaattttagaataaaaaaattctggaagatatctctggtgacttaaatgttctagatacaaagctggtcgctcatggtagttggatgatgtgttcatttctacttttcatttgaatgtgacttagag

SEQ ID NO: 12 - DNA, 599 bp, Homo sapiens
acataatattttactctataaaaattaatgattcttctctcactttagtgtctgtgtgccgatgcctccgtaggaaaaattgaataaatacgcaccaaagtgagaaatgagttgtattaaatttatactgaagtgcgtggtcatcttagtaattctagaactttccataaactattttatgaaatgcatcaacgtttctattttagtgtcaagacaccagaaaaaattaaaaatgattctactgtatttagtcctacgatagatcacacgactattcatcgaagtgaaaatactggtgcyaacaaaagccaacctttcaagaaataatctgcctacagtgtttgatccagtatctatacacataaaaccagctgcaagataagcaatacaatttaaaacagttgttacaatttcaagtgaaattggttgcttccaataagacttccttttatatagaggggaaactcacagtgtggcaataaattcatattcatttaaattagtgcctaaatataatgtgggtggtctcccaagcactccttggtgaattattggctaagaggtgtttagaaagtctgggagggtgggtagtgatcatg

SEQ ID NO: 13 - DNA, 599 bp, Homo sapiens
ctcaacctccttgttgaaggcatcccggcctttttggttctggagcctccacctgcctcctaatgctcccctaagtcagttagcacagcccccgaccctgcaccacctcgcagttgttgctggtgtgcggttctcgccggagttcttagtgcttcacggttatcttttgctgggattgttgaaagttaacctcccgttaattcccaaagtgttacttttactgctatactttaaaatttcttcatttgccactttttaggtttgtggtcccgggcaagtttaatttcttttcaggattcycatttgtataatgaggacaacagctgttggttcaggattgtgaagcttcaatgagataattgtttagcatgtttttattccatcatgaacaccatttgtattgtgttttacagtttacaaagtgctgtcacaggtgttacttcataccttaacactactcctgtccagtgaatattagtagcctcatcttgttgagaagactgaggccaagaggtggattgactaggcaaagtcaacacagtatgttaggtagaactccaacctaaacagctattcttgaccctaggttgaccatatgg

SEQ ID NO: 14 - DNA, 599 bp, Homo sapiens
ctggtgatagagtctttagccagagccctggatgagatcacctaggaagaaaagggggatggcaaagaacatagattgagcagtgaccatgaagtcacctgtcaataaatgcagaaccctcataacttggtcaaaagcagttttggtgataggtttgagtgtgtttgagtgcgttgagaagcaaatgtgaggtgaggaatgggaaacaagtgtgtgtgtctctttcagtaaattttatggaaagagaagaagagaaatggagccaaaggcagagaataattcagtaagataaaagatctygtagcatatttgtgtgttgatgggaatgacccaacacagagaaggaaaatgtggtgtgtgtgtgtgtgtgtttattgggtgtcagggggacagaggaagtagcaggcacaactttgatttcacaattctgtgctggaagagagataatagttagagatagtggatattctgtaggctatttgaatagctatgtagtctaatgtgggtaacataactggcaaatatttattcattgaaaactgtatgcctaggtacaactttaaaagattacagtctacttgggaaaacatataatttct

SEQ ID NO: 15 - DNA, 599 bp, Homo sapiens
tctctttatcttttatctcccttacctaatccttcttcttcaacaaaatctgtttattcacactttatttatgatagtctttttgccttcatgacttccttccaagatatacccgtccaatttattcaaatactacccatctttagcttattacatttcttctattattctttgaaactgttgtaatcccatattttacttgagttttcacaggagtgcatgaagtggtggtggctgtgttaattcttttacaaccacattgtaatttttctgaatgtagggaccatgtcttggacttcyttatactgagcacgtaggtctataaaagataattgcttgcttgaatcaccttcagttcattcatgcaacaacatttaatgagcaattactttgtgctagcaggttaaatataatacttctgaaaaaaaagtttttcaaaattatttactcttcctaaaacttacatgtcaataccttagagaaaaaaggccatttctcttttcacctctctttttgtaactttgcacagttaaactctgtctcactcacctggctcactagtgactcagagattttgcttgggtcccaaatctaagatc

SEQ ID NO: 16 - DNA, 599 bp, Homo sapiens
cacagaggcaacttgcctcagagtatatgatgccttggctggggatggggtagtagacctagttcccaggccatcccaacttggacccacgagatgtcccctgccccagtccggctctcagggttttttgtactgtgtcaaaggatgtagagggcttcactttccctggaaatgtgctttagattgctttttgggggaatcttgcaaactgggatgtttggacttctctcctgccctatgctgaatttggagggagccattttctgtctgggaaatgctttagatcttggagccactggrgatgttgaagaattgaaaaaggccaggtggggaccctcaggtacccacagtgcttctccccggtgaggaggcaccagtgaggaagtggggtctgatatctggtgttacctgagaaaaaggtgggacggaaaaagaagagccttctaactggtccagtaaggccactctgggacaaatcacagagtcatccaaggtggcagttatcttagctataaggtggttttcagtgggaacttttcactgaggaccttcgttggttgccagcagtggaagttcaagctctttcctgccctgttgta

SEQ ID NO: 17 - DNA, 599 bp, Homo sapiens
taaacctatcgcgtgccaggtactggttaggtgctctaaatggtgatttgtttgttttctctaaaacaaacagggccttgctatgttgcccaggctggtcttgaactcctggtctcaagcaatcctccaacctcagcctctaaagtcaataagattataggcgtgagccactgcatctggctaaatggtgataataataacactaactggcattggttcagggcctactatgtgccatgcactgcttaagccttcactcatcggatccccacaattccaagaagctgcttttagtactgytccctttacattgttaagaacctgaaggttcttggacttgtccaaggtcatacagtagggctgcttgttaagacccctgatcccagccttctagctgcagggagcacagggaccccagagcctggcaggggcagcatgcagaggcaaggcctgctgggagacaggtgcagggacggggagtcagttaggaaaggagctcaaacagggttcggggtaggccacaaattccccctagggggatggacccccagccccgtcccacctcccacaagccccaacccagcaggggacccacacct

SEQ ID NO: 18 - DNA, 599 bp, Homo sapiens
gtgtatatatgagtggtatgagggcgaattatttttgttttctttttaatgatctcttaaattttccagaatgtctacaatgaccttgtattacttttctaatcagaaaaaaagaaaaaagttaccgaagaagagataagtgaagcacacacaaaaggcagaatatatagtgcatcaagaatgtgtgttttataatctgacagacctagttagaccatagtccatatttcaaatataattacatgtgctcatagctgagaaccttctcctgggatggatgcatttcaccaggtcactgcygaaatgttgtacttttatggatggtgatgaggaagcatctgttttaggtgtggtatttcctggaggcagaaaactgcttgagttagctcattcagtttttcagtggcctttctaatgcatgttttgttttgttttaatgctttgcaaaagattcctagagtggaaattgcctttgcaaaaattataacagtgagaaaattatgacagtgaaagggatctgccctaactgtccccatcttgcttctgggtatctttgctcattcctgggtgtgggccaagctaactttgggagaaattta

SEQ ID NO: 19 - DNA, 599 bp, Homo sapiens
agcctggctgttttcatttccaaagacagggctggtttccttaggagcagatgggtcctatccatctcttcatccctgcatccccactacactgtgggcctgtggacatggctgtatctcactgtggtccaggactcggcaagggttggaatcctggctctggaggacctgaggctgctcagtaagtgttctaagcgttcatgtctgcatttgtagaaagagagtagaataataatacttgtgttgtaaagttgttgaagcattcagtaagctaatctactcaaaactctgtgaatgggkcctggagttaagtaagcactcagctaacatcatctgccatcatggtggtgggcgactaagacggcatccccaggtgactccatattcaatgccattgtctacacttccccacaggcacctgatcaggtaacacgccaggcattagcattcattcaaacagcactgagactctgaaaggttacatggcgtgctccaggtgagccgcttgacaggggatgagcttttacctccaaacccaccccttctacccagaaagagggtgcgttcccagtggccgggaggatgggagggtggaggtg

SEQ ID NO: 20 - DNA, 599 bp, Homo sapiens
gttttatggtgcatatgttacacatcataacctttttcaagtctcttttagataacaggtaggatacaataaaagggcagacatatttatttaagatcaaggaggtaagttgttattttactaaaacttaacagaaattgacaaatacttttatatgtcttgaaaaagtgcttaaagccacatttcatcaaataaaatagattttattttagcttacctattggtaattttcagggctctgaaaaggctttaattaaagagtatcagaggatttaagagcaggatggctgtttgtgtcartattaagccaagatttacaaaggcctattacggttccagtgacatttccttcctatttttccatatcagcaggaacatttcttgagggaatagattgctacttgctgataagatctctgacagtaaaggtaggctaagcaggatgccaggtatgaaactagagaataaaatcttatcaaatgtaaaacttgtaacattgcacctgccatgtagttggtttacctcgaatggtcattgcttttcctcaaaagcatataaagttgtatcctaataagtgttggtatgcaacaaaattatgt

SEQ ID NO: 21 - DNA, 599 bp, Homo sapiens
ccccagacaagtgcccgtatacccagaactttgtatttaatttcatggggttcatgaagcctctgaaactcagcttaagagtccataccaaccaaggtccttataggaagagcttgtgtttttgttttgttttggctttctgcagtctcttaataatgtttattgaatgagagaatttaactaatttccggtttccataatcactttaaactcggtatttgatttccttttccctgggacctgtgacagtgccagcttcatagcctagtctaggcatgccagttgcccactgtggcaatmaatatctgagcctgtggtttttgccttaggtaaactgtagagatggactcatggaatgcttggaaaatttttcagtttatgataatgtgtaaatgtcgagagccaattattgaggaatggcacctcaaagtatttgggtactctagatcagacatgaccatcttggtgtgtgaaattttgctaatgcatctttcctaatagaatatacaatctcagggctagaaagtcttagagatcatttaatttaattcctccagattgattaagtggtttgtccaaggtcattcaactggtatagt

SEQ ID NO: 22 - DNA, 599 bp, Homo sapiens
ccactgtgaaaactccttgaaatcatgtagacaaatgaaagctaaacatcaaataaataacctaaactggtaatactcccatttggctctccttgtgtcttttcatgtgggttagctcttcaaccagattcagcatataaattgagtatgtatggttatatggtgactttcaaaagtaaatactttttctgtggaacaaaagagatggatttgtggttttccaggattccttttccctgacttggtagaaagatagccaggcatagctcagcagacctgaaaataggtgagctgtcaagktgttggcagggagaggctcctctgggtgggacttggggcatcctaccagcgaaaaagagactaagtttaagtgatttcacatttgggctttacttactataattgctgaagtgctaaattgagtgtgacaaatctaacaggtaattgccaagtaggtttcattaccagtcattgcatagataaatgatgttccttatgttaaaaaataaaaccaaactaaagaaaaaaaattggactatatttggaggtagaattcagatattgcagatatgttcatatacatcaaagctgtttcaatg

SEQ ID NO: 23 - DNA, 599 bp, Homo sapiens
cactgaggctctaaggcgcggttctgggctcctttccagggaaactctaggcctctcctgtgctcatgctgcctctcaagtggctcctgcatcctccacactaaccggtttcacaagtttttttttgagcattcttctccctcctccacagccagtttagagctttcttgatcaaatcgcctaacctccgggcgaacacgttcttccctttcctcgggcagggcaccccatcccagccaaggagccaccatcccactatcttcagtcactcccagctgggttacccgtctcatgtgggcytgtttgtagtcagttctgcctagggttggcagatttcgattaaacctcccactaggtccactctcttctgagatctggccttccagaaggcacacttgggtctttgaacaggaatggaaggtgattagggttgtcccggggttggcagaagcgcttggggctacaccagtgtggggccgaaacagctctaagcacctcccactgccccaccacagatccaagtttgagcaagaaagacttctgtgtcctccctgaactccatctcctccgctctgcccacagagactggacctggcttg

SEQ ID NO: 24 - DNA, 599 bp, Homo sapiens
ccccctccttcgagattttcagagtatctccttcttagtaagaattgcttggcttagtatgttgagaaaaacatactaagtatgtagtgttgtgttggaaggaaaacttaactgggtgagattttcagctgagtagctttagctgttactctcatggaccttcctctgggcggttgatacaggttctgtaggaaaaccgctgaatgaaagcagctcctgcagatgctattctgttttcaattgtggtaattcctttaattttttccctttcaatttactccttattctctagtaataatrgacttttgggaggttgcatttatgataaaaaatagcctggggctgtacaatccttcctatcatgaagaagtacaaagttgtaagtagaagcagtggccttgctttgccaatgcacactgaaggggataacgtttgctcctgttcagagttcacggtcgggcgcggtggctcacgcctgtaatcctagcacttttggaggccgaggccggcggatcacgaggtcaggagatcgagaccatcctggctaacacggtgaaaccctgtttctactaaaaatacaaaaacaaaattagccaggc

SEQ ID NO: 25 - DNA, 599 bp, Homo sapiens
cttataacagtccagttaaagttgttcaactttgcaaggggcaaaaggcaagtttaataccctttaccaaggtattaagtatatttttgatgtacaatattttcaacttacaatgggtttgttggatgtgaccccactgtaggccaattgtactttggaagcccactgagacctactgcaacccccagtgagtgacagaaggggtgaaacaaatgatttaccattaatagcagagagatccaggtcaggtggagctgtgccatgcccttccagcaacgtggactgcaggtcaccctttcygaccccactgctatctgctgttaagaaaagaatccccgaaagtcagcacagtcaagagacactctccattcaagccaggaaacgcaggccgtgtttttgaacaataccacttgcttctaaaagcccaatttcgtccaaggtggaaaggctaaaaaaaaaaaaaaaaaaaaaacctgtggaaattcccagccatcaccttgactctatttttaaagtaccactagaagacagagacattctgtataaccaaagagacgcaaataggcctttctgctagttcacaagatgctacaggtggg

SEQ ID NO: 26 - DNA, 599 bp, Homo sapiens
ccctaatgtgaagaaagaccttaagggagactctttgatccttaggtatttgggactgactcagagcactgcacagaagtcactatcgcccccttttcaaatctccataggcaccgttttctggatggttctctttagggcagctttgacctctgtgttccgcaggctgtagatgatggggttgaggatgggggtgaccacagcatagaagagggacaccagagggtcagtggccggatcgtagctggccttagggcgaatatagataaagagtgcggtgccatagaagagggagaccahgatcaggtgggaggagcaggtggagaaggccttgcggcggcccgcaacagatgggatccggaagatggtaacgaggatacgcccgtaggagcccaggatgaggccaaaggggcagaggatgaggagggctgttgccaggataatctgcagttcattaagcgaggtgtctccacataccagctgcaggacaggctggatctcacagaagaactgcgggatggtattggggccgcagaagggcaaagagaagatgaaaggggtgtggcccagccccaccagcaccccacaggcccacgccg

SEQ ID NO: 27 - DNA, 599 bp, Homo sapiens
tcacacttggcacagacaaagtgctgcaaggacaaagggcgggccgggtggcatcagggctagagcggtgaccccgagggacagtggtgacccggggacaacagtgaccccaggagccagtgactccaggggaccacagtggccccaggaggcagcagtgacagaaggggacagaaagcagagtctgggcaggtgcaggcctttcaggattcacactgggcagaggggactgacacctctgtgaatgccctggggtcagcagaccctacccctcctctaaggcactgatggtaaccttgygagtcaccatcccctcccagtctggcctccccctcggcagcctcagccccaggagtcaggtgccagcctggtgctgcggcatggtgggccctcataggcagaggcggccctgcctgtccacgtggggcctcctgcaggcaccccctgcaagcctgggatcactgagcagcaccttgcattcccccagggatctgcatgtgaggatgagggctgggggctggggcctgcatcagggcaggaccgcatggcctcccacccaagccgccacccaaggcacagcttccggacacagcgctggc

SEQ ID NO: 28 - DNA, 599 bp, Homo sapiens
ctttgggaggctgaggcaggtggatcacttgagatcaggagtttgagaccagcctggccaacatggtgaaaccccatctctactaaaaatacaaaaattggctgggtgtggtggtgggcacctgtaatccctgctactcaggagactaaggcaggagaatcacttaaacccaggaggtggaggtggcagtgagctgagatcacgccactgtactccagcctgggtgacagaatgagactctgtctccaaaaataaataattaaaaatgctatgaaaaccaagcaaaacacattgatgggrggaatcataactgtgaccatccattggtgtcccctaccatggattaatgaacaaacaagggcagagggaaaaataccctgcacatctgttggtagaaagcaaaaggtacaattaatgggttatttccctgaggcacagccttacttgcaccaatgaatgtttactggttgaaaatttgtcagtggcagagcttatatacatctcatcctatcatatgaacgcatatagcatgtgacatgtaggctgagacataaaggatgaagaagtatttacatgagtgagaacagtgacgcaaggac

SEQ ID NO: 29 - DNA, 599 bp, Homo sapiens
gctagggagacagacactcagcaaataactgcttaaaacataaaatatatgagacagggaaaagtacactggagagaaataaagcagtggacggggtaggggctctgatgggatggagaggggctgcgtttttaaatggggagcttcctgagtgggtgaaattgtaggggagacttggaggaggtgaggggtttggccaagcaggtgcctgaaggataagtgctcctgagggaaggaacagaaggtgcaaaggccctggcaggagcgtgcctgtgtgctgcggggcagggaggagccacygtggctggaggagggtcggggaggggaggtcctgggctcacatgtacacatgtacgcatgtattcgttcattaatttgacgtctacctagccctaccaggggccaggctctgagtatctgtacctcccaccccgaccctgcccaacacagaggcctgggagggggtggcccacctgaaacacgatcctggatttctcaagcaggtactgggaggttatggcaccagagatcacgcccctggggtggggagcacagtcagctctcagtgagggtaggagggctggtgttggctccgaggg

SEQ ID NO: 30 - DNA, 599 bp, Homo sapiens
acagcgaagggatggcgccgtgagccctcagcgcggggagacacacgccgggccggggcgccaggagcgggggtggcggggaggggagggagggaaggctggtgacgtcagtggttccagttccgcagcagcggaggccgcggccggggacgtgagacaccagctgacgcccggagcctcacaacctctgcacacacattctcctgagggccggtgcctggaaacaaaggccacccccgccggccggaaacttctaggctggcaatggagatgggacatctggtgaccccctctgtatcytcctctcccttggggtcctccaggaagggctgcgtgatcaatgacccacttttttgttttgtttttgagatggcctcttgttctgtctcccaggctggggtgcagtggtgtgacctaggttcactgcaacctccacctcccgggttcaagcgattctcctgccccagcctccctcgtagctgggactgcaggtgcgtgccaccgcgcccggctaatttttttttttttttttgagacggggcctccctctgtcgcccaggctggagtgcagtggcgctatctcagctcactgtaaactc

SEQ ID NO: 31 - DNA, 599 bp, Homo sapiens
taacataaaatttactggctttctcatttttgatttctctaaagtcttactatttaaagcaaaaataataacagattttgtgtctaaagcatatgtaaaattgtagtgtataaaaatagtacataggtcagagggaggattggagatatatatagatatatagagagagatatatacacacacatacacaaacacattcttataatatgtaattttaattttatttttccctttattcttatcattgtatatgtaatgtttttgcagcccattttttgccacttactatgtgttgaaggrcatgaaccaatcacaatatttgtaatgacttcatgataatccctctgtgtgtcagtgaaaatgtattcacagataaaaggctcctgactaactaatttaagcagagaaggactttgcacaaggcattaaattgcttaatccatgacaggagtgagacagctggatttaatgtctagaaatgacacccaaagacagcctgcaccactaaaagcccaggagagttgcttctttggacttagcattaggccacttgtattagtcacagttctcctgagagtattacacacatctctctgtct

SEQ ID NO: 32 - DNA, 599 bp, Homo sapiens
gtcccagttacttttgcagcctctcttttcctattttgccccatcccatccccacaaagaactacacattcaaccaaaccagactcctctttccaagaacacatccccaccttttcgttactacctatttttacccatcctatcctctttaaatccttcctcttctacagatttccccaattctccaatcaaaagaggtctcattctaccacgcagcccatgcctctgcactgccaagcaaggcagagcaccaacaaggcaaagtcatgcagaagtggcagcaagatctttggagtcagrccaattgagctcaaatctgattcagctgcttatatgctgtgtgatcctaaccaaataactaaatgtctctgagcctcagtttccacatctatagaacagaaacagggcctggcacacaggagtcctcaggagaactgggggagcaatagctggcacaatagagacttgggtatctgtcttctctccccctcccccgtacaaaatgagctccttgaaaggcaatacaatgaagcatctcccagaagaccttacacatgaggggacctcaaaaatggagttgcagagttatctaaaagcct

SEQ ID NO: 33 - DNA, 599 bp, Homo sapiens
ttcccgctgccacaccccaccctgggcccccgcagacacgcatctctaactcagttgggcccagccttctggatggcttggggtaggccatgggcccacctggggccaggccagcccctggggcagctctggaagagcagtgtggaggagcacttgcttgcagcctggcttcagcctctggcactgctggagtggtccctgggagcttctgcactgtcggctttggggacgtctcacccacttgggttacagtaggccttccccacccagagagaagtgtttccaccccagagacattgyctgtcagcccctgaagtgctcgcctcccccagtgcccgtcaccagcccttcctatctgtggggtccaagtcaggcttcccctgcggccaccagccatagggagcagccatcagcccccgagtcagaactgcttctgtctgtccatacctccaggctctcccggagagggggacggatatttatttcctaaagtttgcacttaattgtgaggattctcaggattgttgggggctactgaaaagaggaatgtgttgaatgtcgcgtttgctgtccactcgtcctagaagtttagtgttttt

SEQ ID NO: 34 - DNA, 599 bp, Homo sapiens
gaagaggtaagggaacaattcggtatctgtgttttatatatcatggatagtcaagaaagtcattctaggcagtagggaaatgtgcacagaggtactgcaattacatgcgcctgtaaaacccagaggctggcacactggaagggaaatagtggagattcctggcatctcctggccacttacgctgaggtattcccactagctgtgtgcccagcacttcctctgcatgcctcagatgcatttgacaatctcaggtgaactgcacttcagggtcaagggaaccccggccatggttctaagaarcaactcccattttagtatcacctacatttgaaaccacagagcactgtccaggagaggtgatggtggtgggtctcctcctttggctctctggcccatcagctgatactagggagcgctatccctcagcccacatttctcagcatggtaagtctgagagtcttgatgattaaaacacatttttgtgtgtgcaatttcataggttttctaaactcttgaaacccttgcatgaaggacaaagggaccccacgttacacactcctacttcctggagtatgctaagggcttcccaacactggtgc

SEQ ID NO: 35 - DNA, 599 bp, Homo sapiens
ttttagtagagatggggttttgtcatgttggtcagactggtttcgaactcctagcctccagtaatctacccacttcggcttcccaaagtgctcggattacagacgtgagctactgcacccaacccgcctcaccgtttttaaatgggttaccacatatgtaaatagaatagctcaataaatattcactccataactgcaggagttaggggaaagggaaagaaggtaagacaaggtcaacagaagacctgcaccccagaaaatccaacccccatggggcacagtccaacgtgggggtgggargaatttacatctttgccaccaaagaaacaaatctttgccccacagcctgggattaccagggcaaccctcaacgtgagggtaggggaaaacagaagaggggatccatttttcagggggaacatgcttgtcttggcagtttctctcctcaaagatcctgagacctccaaacaagaccctcgggccccggttggctggggagcgttgagcacgcccaaggaacgtggacacaagcccctctgtaatggtgtgcctatcaggggtcagggatccagcctggtccacggtgaccccggcgggca

SEQ ID NO: 36 - DNA, 599 bp, Homo sapiens
ctcctgggctcaagtgatcctctcacctccaccatccaaatagttgggactacacatgcatgctgctattgctctgctaaattaaaaaaaaaaaattagagatgaggtctcactatgttgcgcaggctagtcttgaactcctggcctcaatgaactcctcaaactcaaggctcacacatcagcttcccaaagtgctggaattaaaggcatgagccaccatgcccatcccacactttattttatacttactgaactgtactcaccagcttcctcaaccacaataaatgattcaggtgtccrtacaggaagtggaggggggatttcatcatctatccttggagcagttgctatccaaaatgtcaaaaatattgtaacaattgttaattagaacaatccaaaggaaattcttatattctaatattaaatataaatttaccataatttatatttaaattccgttgaagcaacattatcagtaaagttgacacttgttcattcaaggaaaaagcaaaataaattctcttaaggtacaaacccaggaggtttttgcttattctttaacatttattgttttaaaagctcaaaagtagggctgggcg

SEQ ID NO: 37 - DNA, 599 bp, Homo sapiens
aataaaatataaagaggtgacattgacagagttgcaaataatagaaactgttgtttcccaaaggcaaacccacagttgtccaaagagcaccataagcatgtattttacagcatcagataagtgtctggcttcctcctttcagctgactgaggcctctctcctatgaggaagaagctttgagtcctggccattcagcatctgccttgtaatgagggaaaaaccaagaaccaaaaggaattaagagactagggggaaaggaaaacctttttcagcctgactcccttgctaagtccctcactkacagtattttagctacctggatgtcgcatgatatgtgatctgacagatctataatgaaaacagagacactagggaacaatgtgattggaggaacgtaattacggaatgtggaacagctttccatgggccctggtgtgtctgcaaggcagtgtctgccattcctctgggtcattgtcaaggacagtgatcagccttggagaactgaggtcatgtctccctcctcagcaaagacaggcattgttagaagatttggcttccctgtgttcagggttggtctcctgtaacaaaacctgctgcat

SEQ ID NO: 38 - DNA, 599 bp, Homo sapiens
ggaagactacatttcccagtctgattgtatctatgtggggctatgctaccagttctggcaaatggactatgtaccagcagcacgatataccacttcatgcctagcacctacaatctgcaagacagcatctgcattctcctctctgtctactgtaggattatcagtgtccagcaaaaccaggacattcaccaacatattttgtcaagtaagcaagaaataaatttctattgtgttcatgtactgaaacttaagggtttctattaaagtgggatgattacttccgttgactaatacctagcmcaaactacactcaatcaagtatcaataataagcatattatttattatacgtttgaaagctggaagagaattattttgttcaaagaaatgtggatcaagtatcttgaaagcatttatcagtttaataacatttggcatactactttgtggagaaaaatcacatagggaacagagtactatacggtaaatgcaaagtccttctataatattttctgttgagctgacctctgctttttgatatgacaaagtcaagaacaatctgaaaaaagtatcaaacacatatgtaggtgagggtgttcc

SEQ ID NO: 39 - DNA, 599 bp, Homo sapiens
ctgggttgctggggaagggggtggaggagagagtcagtctctgggtgggcagtgacatcctgcgtaacagggaagctgtgcatctggctctgtggacagcctgtgaggggcttaaggggtactcttcctaggtctctgctggagaaggagctggagtctgtgggcatccgcctcaacaagcacaagcctaacatctacttcaaggtgaggcacctctctggccttcaggccaggggtgtggcagttttgagactgcattggctggcggctgaggctgtgggaccattgctgtgacccctyggtcagcagttctccccatccctttctcctctttcagcccaagaaaggtggtggcatctcctttaactcgacagtcacgctgacccagtgctcggaaaagctggtgcagctcatcctgcacgaatacagtatccttccctgaaagacacgtgaaggagggcagccaccaccgtcagcgcagcggggggactgactaagacaggaggcctcatggagcagggtggcagcaggtcacccccactgcccctgctgtccattcaagtagcctctgggcaagctccagactgccaggtctgcag

SEQ ID NO: 40 - DNA, 599 bp, Homo sapiens
ctcataacaataagaacttgtggtggtagttcatagaattaatttctatagtgtttgagtgaaattctatctgtaatagggatagctctataaaaattttagtcagtatttagagaaggtaatattttttgagactaaggatttattggcactccatgcttcctctttgttgctgccagcatcagacttgctataactggaggaagaagggggtatttactggatcatctgtaattgatgtaccgtgggatacaaactgtgggttcaaaggaatgactttctttttctttctttttaacragggcaattgactatcaagagggggaaaaaagtctctaataatataaatttttagaaaattaatttagtgtattatttgatatatatggagttcttcaaatagtgcttgcacttgctgagggctacttgtgttatctggtattattactggtatttttagagaaattgactaggagtggcaaactgtgctagccagctgtgcctacttgaaagggttgttatgaggctcatacttgttaatgtacagacaaaggctctctccttgaccaaactttagacaggctcctttgagccctctt

SEQ ID NO: 41 - DNA, 599 bp, Homo sapiens
gggtggtgctggggcagtgctgagggagccagtttggaagtggcgacctcctaccctcaacttcccttggtgggagtgcccgcagctacccaggcgtgccaggggcaggcctgggattttagcctagtgtctttggaatatctagttcagccccagaaccacatctggcttgcctgagccaagggaaggggaacagccgtggcacatctctagctgctggccctgaatggcagtttccaggtccgtggcactccagcttctggagacaagggcagatcgcctcagagaggcctgctaccrcccagccctgccctcagggctgcagagctgtccctgccctgccttctggccaccaccctgtcatgatgccattgcacagagccacctccagtgtgaacaggaagattgtgttggagagagcaccagcctccagctacttttgggtctggggtttgttcagaacagtggctggtgaggctttgggggaggggagccgagaaggggactagagactggggggtccctgggggaaaagccagcttctccctaggtggcagtaggcaggggagtggagactccctgagatagcaacacctcct

SEQ ID NO: 42 - DNA, 599 bp, Homo sapiens
gcctagacttcactactaagcatgtaagaaacctgcgcttgtaccctctaaatatgtaaaaatatttttaaagttgcattttcaaggaataccactagatgtcactgtggttttgcaggttgcacaaattttatcagtaaagacataaattccttggtggcctgcctcttgactttgactcatttctagactgggcacatttagccaatttcaacacatatagcctttgtgaagcctacaaattaaataatggtaaaagcaacgaggaaggaggcagccggcttaaagcttgtttctgcrattacaagccgtgggagccgggcagttactgctccgtctctggacttggtttccacatgtggaggagacttggattgcctgctcttagtctcttcaagctctcgtagtctatgactttaaactgatgttttttcaactaaaattatttcagtgccccttacaagatttttgtccaacgacattgaacccactactgatttttctgttttgttttgtttttaaactcagagcttttatttatatagtacttgtttttaagttgactcagctttttaaaatgtaaataaagaaaaaaatgt

SEQ ID NO: 43 - DNA, 599 bp, Homo sapiens
tccattactgttattatttgaccaggggaatcatcttaggctttatggaggagttgacattttcaatcagctttgagggtgggtaggctttggacagacagggatggggcagaagaacattctggactatgggcacaacaagagctaaggctaggaggtgggactgtgtaactttgagatggagaatagggaggagacctcaaagcagaaccttaggctctaagggaaacaagacagaaggattctgctgacaagacagtaaagtagcctgctcatctggtggtaggcactgtgtcagcrttctaggttgtaaatgtaggaagtaagcagatcagaggtttgctcaacaacctgcctagtgagccaaactgcttgctcttgaggccatgtagtccttctggggcagctcttcaaaccctagtccacagaggcaggtacccagttctttcagtgatgccccaagccagataagaaaatgatgttatggagatctggaatcctcacccaaaagtccagctttcattctaaatgcatccgtccatcaggacagtcttgtgtgtgttcagaggtgagggatgaaagagaaacttcacagcaga

SEQ ID NO: 44 - DNA, 599 bp, Homo sapiens
agtgggctgggtgtgtaagcaggttctcagacccctgggcagcttgtgtggtgtgctttatggcagtagcagtggtggagcaaccaacccacttagacccagttagtccatgttggtgttgctggtggctgccatggattggttgaaatagttctcagtcccccagctgcctgtagcagggtagtgggtattgtcctaagtatgcttaggagagcttggtccatcttgttcctcttctggctgggtggcagctgcagctgtgtcaccttcaacttggcctgagggcaggcacagcccagmattaaactctcaaattgatgccttggccttgagactagagaggttggggcccctcccaggcaagcagcatgaacaagaagctgtggcgggtgcagtctgttcatgtctcaatctcaacagcaggctgcacagagtggtaggcattttctcatggggtacgtgggagtgcctgatctcgcctctccctccttgaagaagtgcaacagcagcagccatgtctgtagatctgtggtatctagcttctcaaaatggtgtccagctgaagctgctctaggcttggatgtctgtggaactctgtg

SEQ ID NO: 45 - DNA, 599 bp, Homo sapiens
aggcgacttccggagtggggccgcctcatcagtcattgtaccctgctatcttccagagacagaagccccagaagcacacaaaccaccaatcagaagaaaaagaactactgcatttcatcgattgtaagatacacatttttttatattacattttgttatctctgaaatctgggtgcatcttacaactgatggcaggtcagtttatttggcagctgttttcctttcttagaggagctaaagtaaaggtgcctcttcaaatgagtggcatgccagattcaatgaaatataggatgacttacrgagccctaattatgctgcaggcatcattttggccagttagcacacaccatcatacccctccaagagatctgcattacaaaccacaaaagcggctcagaggggttcagaaaacttcccaaagtcacacagcctgtaaacagcagacgcagcattcaaacccaagtgtgctggggcccaatgcatctatccctctcactatattggcctcatctcctttccttctaggtgaagtgatgggggacaaggccaaattcagtgtgtcatttcggtcctcaggtctgtgggtcctggataattct

SEQ ID NO: 46 - DNA, 599 bp, Homo sapiens
tatgccagccgccatgccggaacggagggagctgtgtccagcctggccgctgccgctgccctgcaggatggcggggtgacacttgccagtcaggtgaggctggctctaccctggggggccctggaagggtcctgggcacctccttgcatgtcctggggttactgctggcccatgagtgggtggttgtgaagggagctgtgtgggcaggacccctcccgcaggcatggccgcctgacaccccgtttcctgcagatgtggatgaatgcagtgctaggaggggcggctgtccccagcgctgcrtcaacaccgccggcagttactggtgccagtgttgggaggggcacagcctgtctgcagacggtacactctgtgtgcccaagggagggccccccagggtggcccccaacccgacaggtaaacagccctggctgtgcctggcctggggaggcgggcaggcagtggacattgccgtgtggctgttaggcatggtggggggcactggaatctgggcggaaggcggtggggactccctctccagggagggaggatggggagggaggataggtgggttcccgagaactgggggcaggttgcccgga

SEQ ID NO: 47 - DNA, 599 bp, Homo sapiens
acattgcattttttacagattgaaggtttgtggcaaccctgcattgaacaagtttatttttctaatagcatgtactcactccacatctctgtttctcattttggcaactcttgtaatattttaaactttgttataattattatatctattatggttatctgtgatcagtggtctttgttgtcactattttagttgttttggagcatcatgaatcatgcccatataagacaatgaacttaatggataaatgtaggttctaactgctctaccaatcagccattacccaatcttttcccatckgctggggccttcctatcccctgacatacagaaatgttaaaattatgccaattaataaactacaatggcccctaagtgtttaagtaaatggaagagttgcaagtctctcattttaaatcaaaagctagaaatgattaagcatagtgaggaaggcatgttgaaacccaaggtagactgaaagctagtccccttgcaccaaacagatagccaagttgtgaatgcagacaaagagttacttagacaaactaaaagtgctactccagtgaacacaaatgataagaaagcaaaacagccttattg

SEQ ID NO: 48 - DNA, 599 bp, Homo sapiens
ggcattccatgtacccactaggggaagctttatcaggctgcatttgccttatgcagaagtatgcctttgttctgagaaatggcctcagaacacactgttgagctgtctcctttagtgagtgtcatcctctcatccatttcaaaagattgcagatgccgtcttgaagtcctgcttgtaagaaatgcaggggttgggaatggaggagatgacgatgtagttttgcttttagttttcccaaacctgtttacattttttaaaaatgtaaccaacccataattgcattttacttgtcgtggttcratctgattgtattgtcgaaggactatttttgaacctgtccaaataaactctgcaaagtatttccacaaggatgtgactccatggcgcttggacagcctttatcctgaatggtactgagacacaaatgttttcctgttgcgtgctttctaattacctaaatgcaaacggggtggaagggagaaagtggaaattctaagtaccaaaaggaatgttaaatttagaataataagctcatctggaagctatcaccacttttttcttaaagtgcgctgcaagcatcgcttccccttggacaccaa

SEQ ID NO: 49 - DNA, 599 bp, Homo sapiens
cggttgttttaaataaatggaaccttttggggagacagggagggtggatgtgtcttctgattttatgagtctcttttgtttctagagaatctttcgtttctgttgcttgctttttcttttcctcctttcaccatcaagcatccaggggaggtgaccctccaagttacctccctccctccgaagtggtgccctcccaagactttggtcctggcaacttttcagccccttctttcatttcctctgtagccaaggatcagacttgttctcagtatttccacacttagggtggtacactctttytgggggtgattttcatctgttccccaaccaggctctgctgcctgactctcttttctggaaaggtttctgctgctttcagacgttcatttttctccctatgcattaattgaagtttagagtatatttcgtgtcctggctatgctacaggcagggctggcttcataggcatgccaccaacgcggctgcacagggccctattctcagaagggcctctcacctggggtttaatgcttggcagtcactgtcttaaaatccttaataatttagtatttgaatttgtgttttgtaagtgaagtctg

SEQ ID NO: 50 - DNA, 599 bp, Homo sapiens
gaggatttcacatttagctgatgaatatgttagtgaattgaagaaatcctcaaaaactttagcaagtggaacattcctgacgtagtgcgttaactggagtaacctaatataataaccataaggaaaaaacaagtcatgctccccagagactatctatagtcatagaaagaagatgtggaactttaagtaaataactaattggtatgatccaaagtgttgtcttgactactagtggggtctctatttggaaatatgaactgtttttgctttgaggtttttcacacaaagaataatcctacrccagtgaaatttccgagtgtttagtaaatgacagagtcttggaaattgggattagaaccaagcaatttcaaggcccatctgcaagacaaagacaagcaggtaataaaacgccctgctctgtttcctctttggcctcttttagtttgtgtgaacttttaacccaagaacattaagtttaaaatcaaattcaatttgaagcaattttgagccacacaactgggtgaggataaatggctctatattaaaatgatcttttccattagaaatgacttcttggatatcatttggtctcattggtt

SEQ ID NO: 51 - DNA, 599 bp, Homo sapiens
ccaaaattataaaactctctgggccaagactccaaagcatctaaagaaaacagaagaacccagaaagtcctaataatacatgaagggtaaatatagcatgaagggtatggtttttaaatttcatatcctcattagtgaatcagcagaatgactgagacataaccctagattttcctatgacttttggtaaaattttatgttaccaattttattcagactgcaccagtgtggtctacatggtaagacaaacagcttcagagtggaactcttaaggcactgggtgattcacaaattgaaccragtcagatgcacagtagttgtatattaagtacatgcacaaatgacactaatggacattgctgccatggcacgtctcagtcatctgccctggtgctgttctttttgcaatatcagaattagtaacataaaacagagggcctggataccaaattcatgagtgacatgttgttcggagggaaagcaagtaggttgaaatgccatgttagtcaatgccatcatccttatctcgtgtaccatataatacgtacacataagccatgctgccagtagtcccttccctctctgttccaggccttgga

SEQ ID NO: 52 - DNA, 599 bp, Homo sapiens
tgaatggcacggccaccctctgtgcaccaaaggtttcacaccttcacctgagagctttaataattctcccatcttagccaagaaaactctgccacataacctcaggccttattaagtctatgaacagaaaggtaactggagaactgcaataattatctggttaatgagttaaaaatcactcttgcctgaaataacctggtaataagtaagtcttgctatcatattcattaaaaaagaacatttctcagagagccaaaaggaatgaggatggtgttcaaatcagtttacaagtcagcattyggggcactgcatacaactttccaaagtaaaaataaaggtacaatgtatagagaaaagccaccaagtaaagttgaaggcacctcttaatttcaaaatgaggatatctagcattcattaagaggtcaccatgtgccaggaatgaggcttatagttttatgcagatcattttaccattccttaaaacacctaaatgtggcagttactattttcatccccatttttcagatgagaaagctggggctctgaggagttgcatgacttcctcaagggtatcctgctgctaagtggtggagctagct

SEQ ID NO: 53 - DNA, 599 bp, Homo sapiens
tgtaactttgccagagaggggctatctgagggcttatgtagttcagctggacttctgaatatctgcacggctcacaaaggaaccattctgagacagcttgcccatctgtgtgcattttctctgaaaacaaaaggatcaagagatgaaagaggacaggatggaggaaaagagaaggctcagaggtgagaaagaaagacatggagacaggacagacagagtatcccacactctggaactcctcctggtatgatattgaatggaattgctcctggctttacctggtgaatgatatccttcccragcccatgaataatctgacagggatcaaaattgcctctgagtagcagaggccccagattactcacacaacattcaggtatgcaaaggaaattagctactttatttctttatacttgatctaattttttaatgagccttccacctgtttgggtaggaagaaaaaaaaacaactatatttagtccaatttagtttaaagttagtttggcccaattatttatgcagaaactctgtattaactagtttttctgctcagttcttcacagctatcttcttcctttcttgcttctctctgggtccc

SEQ ID NO: 54 - DNA, 599 bp, Homo sapiens
ggtgagatgacctataggagcaagtggaggtaggctaggaggctaagggctttctctccagtggaaaggcttttagttgaatgttttagtttttcagttctcagtcacatcttctgtttttatggtatctgagctcaccactggaatcactgcttcagtttccgctgaacagtggcctttggtctgaaggtagaggaaagcatacaagaaggaacagtggttttgctgcatgggatggaggaggtctaactgctttgaatagacttttactatctttctcctttaagctccacaattcartgctctcatatccaacttctgtgtattgtattatcccagtcttgagcctgtgcattctcttgcagcaaattaaattgttcctcctgtttcccttggagccccaccttttctgccagtctgtgtgttgcataatccctcctgtgctggttactagttacataactcatgtgactcaactcgcagccaattagcataaacctaggtcttatgttcaaattgatagtcaaattcaagggacacataagctttatttggtatgacatttgttcagctttaaggaaaatataatagaagcgata

SEQ ID NO: 55 - DNA, 599 bp, Homo sapiens
agccagcatattatcttctccccattaaagagctttaggccatctgtcccatctagactaccagctggtatgatctccagaaatattcctcaactgtaggaactaattgggtttttccatatgtataaattgtagattaactgtatttaccaataaaaaaagttcaatttcatctttgtggcaggtgacctgatcttaataatgagggtgaatctcacaggcaggtcctttgggatggttgtaatgtttctactatcaaagctgttgcttgtgacaactctgggacattttttcttatayaaaatcaaagccagagttatgaactcatttaaaagagcaactgtaacagaagaaaatgtaggtttagaagaagtgataatgacatcaccagaagaagggacactgcaaaaatgtaccctaacatagtgttcataatgctgttatacaaaacatgatggctctccaagataaaattaaccatttccagataggattaaacataactctgaatatctatggacatgtgattaaacattatgaattcaacaaccaagctataatataactcatctttgtaagggagacacttaaaaagaaga

SEQ ID NO: 56 - DNA, 599 bp, Homo sapiens
taaaatcaatagtgttggttgtgacaattatgatgtacttataaacttttctttaaaacatttgtgaatctgtacacttctcaacaggacacactgaaactgcctgttttatgttccctatacagtagcaatgctcaggagtagtctgctttaattaggcctcaaggagttgagaccgcctgccctgaaccccaatgagcctgtggagcacaccacagactttctagtgctggtttgtgtgagcaagtgcttacctcagtaaggcctttaatcttcaagtacccacaaaggtaagagttycccgtgtccacgtgctgaaaagaaaggatacacagcctgtcacaaatagataaacttactaggggacatgcaggagggtactctgcaaatcctgtgaggccagactggtggtcagtgaacacccagggcagtccaaggctcacccagtctctggaatcttgataagttctccccacacacatgcctgttctgccaaagcaaactgatcccaagaccctctcaactttcttcagctgaatatcatgagactgaagtgtctttctgaaatggggtgcccactcaaattcattttctgtact

SEQ ID NO: 57 - DNA, 599 bp, Homo sapiens
accttagtctgccaccctccttttaaagaagtgataagatgggatgaatttggttcacccagcctcatactgtgtttcatataaaaacacatatccaaaaaaatatatatatatttttatatatatatttatttaaatatatatttaaatatgcatatatatttttatatatttaaatgtgtatatatatttaaatatacatatttaaatgtgtgtgtgtgtgtgtgtgtgtgtgtgtgtgtgtgtgtgtatatatttacagcttttttctcttatttctaggagtcctcgccctacctrctgttgcaattggcatgttttcaggaggatatatcattaaaaaattcaaattgtctttagttggacttgccaaattggcattttgttctgcaacagtgcatctcttatctcaagttttatatttctttctaatctgtgaaagcaaatcagttgccggcctaaccttgacctatgatgggtttgtatatataattggataatatgttaaccatccaatcaaaagactatgtgtaaggaaaatagggtagaaaacaattgtaattaaattttcttttcactgagagaaatttcatttttga

SEQ ID NO: 58 - DNA, 599 bp, Homo sapiens
gtacacatgcccatgttgaatagagacaggcaagagttatgctttgtgctcttggcagagcagtggagccacttcctgattcctaaactggatgaaagagtgtggtcctggcctgcagtgcatggccactgtcctcaaacactgctttatctcctgtgcacgaggcctgcccagctctcacacatgtaatctagacttcttgtgtgctgagcactttcccctggtaccataattaatgccatctcttccaggacttccctgaagggattggtctgatcttttgggatccccatagcattyacaggggttcctaacatagcgttcatgtcgaagaagagtataatggtggagagacttgaagggctgtaggtttttggggataggtatgtgaacattttcagtctctatagtatttaaatgcacaattgaaacttggcctgctaaaaactttctaaagctccaaacttaaccttatgctccaggtccaaaggggtccccagagtcaatccctccccctgcctgggccatattctccagctcagagaggatccttgaaggtaaggagatgaggaagcaaagcccacccatcctatcaatgg

SEQ ID NO: 59 - DNA, 599 bp, Homo sapiens
aatattaaccatcacattccccttggactatggaggccatcctacactacacagttgaaaatcttcctattcaaccacttgtttccctttggttgttctaaacccttcaagaaaatcttattcaacttcacgtggcatagagaatgaagggctggggctgagaaggcagggcgctcaggccactgtcatcctctgctctgctggggtacccaagtgaggacatggattctggagaggcggaggaggaaggggtagccacaggggtgtgggggcagcccctggagggtgactggatttcaygtttggagtggaacccacttaagatgtcttcagtcgactcataagggatcccaacaagtcaattagcagctgtaaataatttacaatgctgagcaagctgctgaaatgggcagaggagtcatcagtttgaaatcttggagagggagacgtgctgatacatggttggcagaaaagtcactgaagtgcatcttaacaaatggacctcacttctggcgatgtgataatgagcttagtggataacaggagtgaagtgaactagggtacctttctgtctcttttaagcacttaagcctctccat

SEQ ID NO: 60 - DNA, 599 bp, Homo sapiens
cacacatcacaccagggacttctactcatacgtgaaaagctagagaatcatgtttaggtaggcaaggggagagaggtcctggacaggttattgaaggactgattttaaattacaaaatacctctttatgtttagtttgcaaacaaaaggagtcaattagtgaaggcttggctgagctgatttcagaaacagtagggacccactttttgcagtagttaggaagcctcaggatctttttaaatgtatcctaattactagcctaggaaagtcttaaaagagctatagtttgttgggagaaagrgatttgccttctttagaaaaatctatctttggtaatgcacttggttttgacaaaaccaagtgcattggttttgttactttcctttaaaaattctatctttttaaaacattcctggaactcatctgttctttttttttttttttttttttttgagatgaagttttgctcttgttgcccaggatggagtgcaatggtgcaatctcagctcactgcaacctccgcctcctgggttcaagtgattctcctgcctcatccttcctagtagctgggattacaggtgcccaccaccacacccagct

SEQ ID NO: 61 - DNA, 599 bp, Homo sapiens
atcagagtgaacaggcaacctacagaatgggataaactttttgcaatctacccatctgacaaagatctaatatccagaatcttcagaacttaatcaaatttacaagaaaaaaacaaccctattgaaaagtgggcaaaggatatgaacagacacttctcgaaagaggacatttatgcagccaacaaatatatgaagaaaagctcaacatcactgatcattagagaaatgcaaatcaaaaccacaatgagataccatctcatgctagtcagaatggcaattcttaaaaagtcaagaaacaayggagactggcgaggctgtgaagaattaagaacaattttacactgttggtgggaatgtaaattagttcaatcattgtggaagacagtgctgtgattcctcaaggatctagaaccagaaataccatttgacccagcaatcccattactgggtatatacctaaaggaatgtaaatcattctactataaagacacatgcacacatatgtttattgcagcactatttacaatagcaaagagatggaaccaaccaaaatgcccatcaatgatagactggataaagaaaatgtggcacatatacac

SEQ ID NO: 62 - DNA, 599 bp, Homo sapiens
cttagttttactaaggcctttactgcttcttttactccctttctcctgaaagtaatatctcaataaatcattcattgaagaatttccatctcaggccctgcttctaggacactggtactgggagcagtcctaagaaagaaaccctatagctggaattctggagttgaattatttactggccagacggtagtgcatcctgggttggtggagccatgatagttcctggctaagatgttcacccatggtgaactggcagggggtgcacatgaaaggagggacactgactgatcaaatatctcyggcatttcaaaggcatatggaaaagggtaactctatgggccatggaattgttttgcttttgctaaatgttgtagatctagagatcatgataatctcagatctattaattcacagtttgatgtaaatatgagtcagagggcctccttaggagcatttaaagaaatcctaatttctgtaaatggagggcagagaaaactgatggcttgaaacagaacttaattggaagaggcagaaaattttggagaaacttgaattctcaacctagggaagtttcctaagccaaagtcagagttctgata

SEQ ID NO: 63 - DNA, 599 bp, Homo sapiens
gcctgcttgctgttacctagcaggaaggttagagatcaaatggaactgttgctatgaaaatgtgttgttacctgtaacagacatgtatgtgtaatacgtgtttctttagggccagatagcaagtgacgggccaaaatgcaataggtatcttttgagcatcatcacatcaagtctggagtttgttcctcagacacagcagtatcccaggcattcctttatgtagtggtcagtgcagaagcctgagactcacaagcaggcacaggggacatgtggcaacaactcattctcaaaccgtaaaartattgaaatttgacccaggaaatccaagtcaattatgtggggaactcatatagagcaagagccattctcttttcacttgcaaaattctcagcaaaataactgatctttctgcccacttaaggacatgagtaggaggttaaacaagcacagtttgcaggtggggctcatactgaaggtcagcttgagagtcgtggaaaattccgtggtggaacacaaggccactgaaagatcatttcgtcagttaccagcagggtggagtgagactggggtttcaccaagacattcactcactagctgtg

SEQ ID NO: 64 - DNA, 599 bp, Homo sapiens
aaaattttcatcttataggtgattaaactgaggttcagagagctgaagtggcttgcccaagatcacttggctattaagtggcagagctggatcttgatgacatgtcttctgatggctagtattattctctctaactgataaaaacgtgtttctgagttgaccaccctgaccatatgctctagtaactctgcgttctgctttcaacttctaaagggcattggggaaaggcagaaatgccacgtgaactgatacttctctagattattaataagatgctccatacactttttggcatcatayaataagtgacaaaactttcagaatttctgcaagtacctaatgtagaaccatggagaattttagtttatggaagttagtctgcttgtcctctcttttatggtatcattctgcagggccagttaaagtggatttgggcctcagtggaaaaaattgagattctctcaacaagacagagactttgtgctgaaatgcaaattgtcaatcaataccactttatcaggtttctgggtttcccccaaagtgtgagttccgtaccaatatacaacatcaacatgcaggtatcatcaagtcctgataat

SEQ ID NO: 65 - DNA, 599 bp, Homo sapiens
gttattcctgagagggctatgttcctgagagaacaaaattattgtttttgaaactctagagagaactgctctggcaaaagaaatgtatcttttcatctacagtcattctgaggtgaaagatctcatgatcactctggactatacaacccacaagcagacttcaaggatacctacaggaaccccagaagtcctgattgatcacacaggccctaaagaccctatcaacttgtttatcaacttgcaattctaatttcctgagagtaaaacaccagtaaaaaataatctcctccctcaatattytagagaatgcaaagaaatacacagtcactgtagacctgtgagcttttgccaagtttggttttcattattatacatatgaagagaatcatcttcgccaaaatctataaaggggctgattaactcagctctgtagtaattgtttcatattaaatcagtccaaaagtttagggcatgtttctcaggaactctttatctagcatcaggtatgtatctaaacagtatagtatctgttaattttgaagttatataaaaaaagaaaatgtgcattttcacacgtcttagtagaagctctttacagt

SEQ ID NO: 66 - DNA, 599 bp, Homo sapiens
aagctcctcctcctgggttcgtgccattctcctgcctcagcttcccaagtagctgggactacaggcgcccaccaccacgcctggctaatttttgtatttttaatagagacggggtttcactgtgttagccaggatgatctcgatctcctgacctcgtgttcggcccacctcggcctcccaaagtgctgggattacaggcgtgagccaccgcgcccagcctgaataaaggctttttgacaatggtccaggcagccctaggttttagatatgtttttctacagttatagaagggcactcttrtcctggcattgatgagaacagtatcacatgtgttggccttatgtatttaagttttaggtaagattgtatttgaaaagtgagctctagtgcaaataaaaactttgaaaactattaaatgaaatgagccttaaggagtcttttaactttttaagaaatattacgttttacagcacctcttctttagtttcacccatggaagcaccagatgaagaaccttcccagagaacaatgtgtgtgacatgtccatccattctgccttctcctaagagtttggacagctaatccgttcaccgtctaaa

SEQ ID NO: 67 - DNA, 599 bp, Homo sapiens
cacggctttctcccctaacagccactccacccaggggccagaaactgacttcacctctgcactgaattcctccccccatgtctccctcctcccacccatcttcctcctggtgcttccagcagagccaagaaggcttcgccccacctactcccctcctgctgacacggaccccccacacacacacccaaggaacaggaataccaaaaatgatactaccagagtgaagcgtgtcctgcagacattatctctttaatcctaactataatagtccctatttcagagatgaggcaaatgtgagaraacaagtaactttcccaagaacacacaaccaagaaaaaagaaagccaagattcaagtgcaagtctgcctgggggtctggagccggctcccctgctgccccctgccatccctcccttccactcccacagccccattttccacccagaagccctcctggaagcaaccctcacccccgggccccgttattctaagtccagttgttcacaattcattctacaagccctcatgtaatggaatgctaaaatagaactttgtcaccagctccttttttatgttggtctcaaagctggggacaaagg

SEQ ID NO: 68 - DNA, 599 bp, Homo sapiens
tatttttctattattaacataatgaacttatttaagttaaagtcttacatagcataatacagttgaatcatatttttaaatcaaaacttgcaatctctgcttttctgattggattatttaatctattcacttttaattttcttattttgggagttagatttatgtcttggactttacttttaaatttttatgtctcatatgttatattttttgttcttctatttctactttactgctttcatttgcactaggagaatatattttaatgtggcgttttaattcctttaatgtgtttaaagktatatttcttatgacttcttttctggttgttatagagcttaccatagaagtcctagactcatactcatctaacttaattcttgtctgatatagatatattattcatataaagttatattttactatccttccatttgttccattatttttatacacattacatctttaaatgttattaagccaaaaacgtattattataataattacttcatagaagtttgtcttttaaagaagctgacattttttaaaaaatttaaataagtatgtatttatagcttttgtaatattaactttcttac

SEQ ID NO: 69 - DNA, 599 bp, Homo sapiens
agaagcattactccaagagtctagtgttggctctcttcttcttgtggtccccatgacctcagcactctatgcagacaaatatatgaatatatctacagctctagttaggaaatgggcagaagtaactcaaccatgtccccaggaccaagagcagtctctggcatgtagtgggtactcagtaaatagtagggatttcagatgaagagaacattgtgaacagaggctgtgcatgtatgtattgatttatttggaggatacccctttcaggctgctgaactcctcaaactcaggaactgtgcyttttcctcatttttaatttcctgatccagcataatacatagtagctgttcaatacatgtttgctgaatgaatgagtgagtccaaggtcgatgataaattgatgcaaaactgtggcagtccttatataggaatttaggctttactgtatgtgcagtaggcttgcagccatgcaaggctttttctcatttgagtaactcaattgaagctgagctccaggatgactcatgtggctgccgtcagcattgaaaccttgtgccgtcttcctcccttgcccttgctgttccatctctaatcagtag

SEQ ID NO: 70 - DNA, 599 bp, Homo sapiens
aaataaccagttagccctggctgaatgtccgttgtctacagggccacctggagccagcagtagccagtcgccttgccaacttttgttctctctcagcaggaagaaaagagacaaagcagctactccttgacttttcttggttcaaaaagaatacttgaattataacagaaatgggagagtgagagtgaatttcaatattttcactgtgaaagacaaaatacacacacaattaaagagattattttgttcagctcttgcattagggggagaatgttcattaatgaggaacgtgtcaaagaygggagagagagaacctagagttttataaaacaagagagcggttgaagaagggtagaaataggtcacacctgggaatatactagatcatcgcaggggtcttagttatcaacctagccgtttctcagaacggaaaagcagggcgtgaatttctcgaccatccttgctttctaggagcacaggactcagataaagtttaacatttctattgaaggacatcaatatatttaactccaaagtatattttttgacatattttaagatggctgccgcagggccagcagaccaaaatggtcctgcaa

SEQ ID NO: 71 - DNA, 599 bp, Homo sapiens
acattgatttttcttcctattcttttccttcagaagctatagtgatgggggctccaggagtttttaatgtgattccagattaattctctagcagtggaccgggaaggtagaattgatcaaattctaggacccagtctcctgggcaaatcgaagcacaaattagaataacaaaacaaaaactttatgtgtgtgtgagtacgtgtgcatgtgcgtgtgtgtgtatgaaagtaatgaaagcccaaagccaggacctcattttttcagttcccttttgcagggagaacttacctatgggtaccrttttggaggacaggaatcaaggcatatactgaaccctgttctctttgtgagtgaccctgttgccatttgggacatctgaaacagggcatgctttcaaatgcagcatagtgacaatccttttacacgttgctcaatagcaaaattggggttaagaaagtgactgcgcatgaacaatttttttttttttttgagatggagtctcactctgtcacccaagctggagtgcagtggcgtgatctcggctcactgcaacctccaactcctggggtgtgaacacattcttcttgtggcctccctcc

SEQ ID NO: 72 - DNA, 599 bp, Homo sapiens
gtggtctgagtgggcttcttactgtttacttcatacattgagcaaactcgctggatcccaggcagggtttgggatgactgcaggctgctcccacagggcaggtctactcggatgtgcattctgcacggacaatgcaggggaagaaggtggattcatgcccggctctcctcccccatgctctgccttttcttctattcatagtacagtctattgctttctggctcttataattgtcagttatttcatgcagcttgaagggcagcagtgcctaagacagagccaaagagatgtgctgctgaygagcataacttttagtgaatttaccagtcctgaagagaaacatggaacaatgctttagactgtcattgcctcaccaaacatgtttgcatgtttgcaaatagcatcaaacttagagaagagctagtgggtagactttctctttttaaaaaaaatttttttgagacagtctcgctctttcacccaggcttgagtgcagtagcacaatcttggttcactgcaagttctacctcctgggctcaaaccaccctcctacctaagcctcccagggagctgggactacagacacgtgccaccatgcc

SEQ ID NO: 73 - DNA, 599 bp, Homo sapiens
tagcaggcctgcatctggccagaactgctgtgggaccttggaagattgcctggtacctctgggttggcccaccgtacatatcctcttgcttcatgatgggagtcagggtgccccagaagaggcagcgaaaggttggagctcactctgcccctttgctcggtcagtggagagagcggctctggcaaaactgaggccaccaagctgattctgcgctacctggccgccatgaaccagaaacgggaggtcatgcagcaggtgagtctacctgtctccccaggaccctaggctgaacaccctttrataagcacacctcatgtactcccgaagaggcaaggctccttggcccaccaggaagatgaggaggctgagttccagaagggggccttgcccagggctgctttttctgccacccttcccaggggtgtgcctgaggggtgagagttcctgcctgtctgtgtttctgtggcagctcctgcttccagccctccgggtccagttcagagtggagaccacatagggaaggggagtccacatagggaaggggagtcatcccatctctcccacctggcctcaccttcctggtcatggccctggggcca

SEQ ID NO: 74 - DNA, 599 bp, Homo sapiens
tgcatacataatgcatataacagcacacaaactagattcagccatgcaaatcatcttcaacctgcccatctataagtttctgaaagccaaaagataaagcatttcaatattttacatattccaccttattctataaagatacaaatacttcccctgtgtctgccttctgcaggactcaccagctctgtatccttgagccaaggctttctgctttccaacctttagcatctccttgttgatgtcacacaccacgacacgagacccgcccaaggaatcttcttcattctggtactctttggyaatttcttcccaggataaattttgttgggcccttaactgcctcttctgttttctctgatgctgggactgaacataattaaggaaccggaatgcaatgtcacctgtgggaagccaacatgaggaaagatgcagactaaaacaaaaataaaaaaggaagaaacagcatttcctaaaacccaaaggggtaacagaggcacaggagtacctgtgttagctaactggatgcagtgtggctgaaagccaagtcctttcttctttttgttagagacagggactcactccatcaccctagctggagt

SEQ ID NO: 75 - DNA, 599 bp, Homo sapiens
ggagagcaaatgttcagatttctttttctttgacatgttctttttatttttcttccatttagttttgagatgtgggaaggttttggttgaggaatcataaaactcctctggcatctattttatgaaatatgtagcatattctattggaaagaaattgcaagcttgcttttattattattattttattttatttgaagctgacatataaccaatctgcaatgccagtgggggagtctcataaacagaaaggaaaatataaataaaattctgctgaatagaagagatgacagctaaaatgaygaaaattttgtggtggtgaaggcgggctgtttaacttatggtataaaaataaggggatgggtcctcccaaatagttgtaagtagttcagcatgaacccaaatcggccctcttgaccacctgtagtgagtcttgcaaattaccaaccccatgcatctgctccgagcttcatattgatttacaattttctttgttggtcagtctctgcatctcttgaatggactcatgatttgtttacttggtatccatcaattcaggtatctctttctttgctctttctctgtctctgtctttcctcgtt

SEQ ID NO: 76 - DNA, 599 bp, Homo sapiens
gggaatagacgatataaggtcctgattacagtctaaaattctcttggtgatcccagattcagtttccttgctgatgggtttatacctggactaatcagtcagccagcctggcatggctaaagtccagaggggcatacaacccccaggtctctgtggcagtcatgggaaaatgccacggagattgtgcttaaagggaggaactgctgggagaatttgataacctggaattctccagtggctacaccttcaaggtgtgctgtagagtttacactgtcctcacatatatgttgggctgctctmagccaatggctaagcaagcagggaaattggagctggaccattcctgacaaatgaaagacttagtcaatgggcagtctttgctttgagggtccccattagcctggttgaaattctctgagaactgcactctaatcagaggctctttctatgcagccctcattacacctctctctactttcaaaggggtcaaccttgcattgtggtctgaagtctccacacctacctctgctttctatcgtttgttcacttgcatttcctccaacaactgtcctatatatcttgttccttcttgataactg

SEQ ID NO: 77 - DNA, 599 bp, Homo sapiens
gaataggacatggaaccagaagttttcaggcaggaagtaacaaagtgaggcttgtttattcaaaagataaatcagacatggaagttggactgaagcaggtgagagactcgtagcaggaagaccagatgcttaacctccttggatggattctgttttaactgacaggaagacagttggatttcctaaaggagaggcttagcttgtgggaggaagtgcagtggaaaggatatgaacttctcatcatcctggttcagcatcccaggtgcgcacctgagcatgtgagtccccctttgcccaacytccagtctcaggcgcctcacccaccacaggaggggatgtccccagcctcaccgggctgcggcgaggcggtgcagtatctgtggacagcttctggctgatgcctgccacatgggtgggactcggggtagctgagtttccttctccttctcttctaacttcttatccaaggaaaaggaaaagacctgtctggccttccataatcagtaaggtaaattcatccatccacagagaaaataaccgggaagcctacagtgcacattttctctctgcccccaccccctatgcccatgccgcatcac

SEQ ID NO: 78 - DNA, 599 bp, Homo sapiens
agtgcccctcatcccacagggtcatgaagaagagccctagctgaaactcttctccccagtgtccctctccttctaatccttctccattcttctcctgagtctagccttctgctgaagagcccttaacagagatcactaccctatgatcctgcacatccacatctgcgactttaatgttccttgaaaagggtttacataggattgaatggagggagggttactattgtttttacttcttctctaagccatgtctcctctctcacacaccagattttagtggcttgacttctgtgaacacarttttctgtcagacacttattttgttgatttagttctgttggtaaacattggagcacagtcttacagtcatccccccgcagctagaactgtgagctactaggagctaaaaataagagggtctcctaggcatttaaacaaatattgccgttcaacagagtctctttctttattctaagaattaaaaaataaggtccggacattttttttcacatagttaagtataatctagctaaaaatagtgccatgttttaatatctgtaacctagaattcttatccaagcaaaaacattttcacttct

SEQ ID NO: 79 - DNA, 599 bp, Homo sapiens
gatgggtctcacgctgggctctgacctttcttcttcccaaaccacccacgagctgttggaagttggggctgtgtctctgtcatgcctgtactcctctggtgcctactgggtgcccagtgagtgatcaatggatgcaagaagcagcaaatactgttggctgcatctgatatgtggttatcaaatataaggtttcaaatgaaccttttaggcctaagagtcttttctcaacaaatgattcttgaggagctattttcacattgttcaaaaggtttatgtctgctcaccactcaacaggcaacrtctgacattttaactaactgcttcttgtggactcatccctggtgagcacccaggtacctaccacataaacagcttgcaagaggtacttgatgaatgaatgaatccatcaataaacaaaggggctacattgtaacaggtggcctgccagtatgtctctttccttagactgtgagttccaggagggcaggaagattttcgcatttattttttattccctggagcaggcaggaaggtccccgcatatttcttgaatgaacaaagggatgaatgaagacacactcgtacaaatagatgcattg

SEQ ID NO: 80 - DNA, 599 bp, Homo sapiens
ttgacttctgttcaaatggagctcaaagcttatctcccgtgtccataaacaaggctgttcaaacaggttaggccacctggggtcggaaggtattttaaagtattttaggcatgttcaagacattatacggggcccctgacagccatcagatatgcagaaacttatgtacaaacacatcaagtgagacttcactggataaaaagatttccaaagccatctggagttggaaggtattttaaagtcggccacagcatcattgcaggctggccccctgagcttgtatttctgctttgttccacrcgagcttcctacaaccttatccattcatttcaaaatattgaactttgtattttttctaaaattttaaggctttataacagagtttcaggacgtctagtctgccatattcctagaagcagaactctcactttattttataatacactggagtcttgcaacgtgtctttcgcagtggaggccctcacatgtgtgctggatgaatgtgtgacggctgcttttcacatgttaattccatctctcctgtcagatactcttgcatttctttcatgtctgccctagcgtctagcttagtgctgtgc

SEQ ID NO: 81 - DNA, 599 bp, Homo sapiens
aggagaatggcgtgaacccaggggaggcggagcttgcagtgagccaagatcgcaccactgcgccccccagcctgggcaacacagcgagactccgtctaaaaaaaaaaaaagacttcaaaattaccttttaattacaataatatattctggattgtcttcttctgttacttaatttccatatccctcaccctacaacattgtgcctcttctaatagttcattgagaagtcacccagtctcaccaatccacacgataatgctgataaggcagggtgtctcatctcaaatcaccacctcctaragtaaatatactttactgaaataaagtattgtctgttgtggggcttaggttaataagaaataatataagtagtaagtactctaaaatcctggctttgtttcaaagtaaaattagatgtttttggtgaacattcaaaagtaggaaaaaatacactataatttagagttgctttgaacctagagatttatctaaattacaataaatatcagaaaaatctttacagtaaaagcaacttcttataagcagatgctttaaattaagaattcagttttgattatttgtttttctttttgtaaata

SEQ ID NO: 82 - DNA, 599 bp, Homo sapiens
tggaatggtttggccattggaaaagcattgcttttcctaaaattgatatagactatctaggagatcgtggaattcaagaccctaaggaaatattttgatcttgagtttgagaagtaggcctgccttcagttctaagagagagaataaaagagcaatactaaaaaatctggatcactacaactttaagcttctggaagtggcctcaaacacctgcccagattctccatagtgcatagttcctttctcaattcctgctgagatgctgttcacctacctgagattgcattgttcttttgcttytttgctttccagttctgatgatattacttcacctttcctcttccaagatatgaaataataaattcaaagaacttccagacaaacccggcccccatcacccacaattgatccatcaaccaagctacacaacatctggagatttggagtagcctgtgctgcttgcaatctgggtttttttttttttttgaatgaggaaaaataatgcaacagttatgaactagaaaaggaacttggatgtgtatctgggggtagacagagtgatgatgatgatgtcccctcagtggaattttatttctata

SEQ ID NO: 83 - DNA, 599 bp, Homo sapiens
caaactctgggttatttttcaatcccctgggttaagtcctgggtgattaccacagcatccctttcctctagatcttgctgctctacaaagccatacagagcccttcagatgttcaaacacagaaatggtttcataataatttatttgtaacagaactctgttgtttgcttttctgagatctgctcactcaggaagcaagattccttagggcaaaatctctgtgcaaggcacaaggtgagaggctcagaacatattgcaccgatttaaataaaaaaaacctctccttggcctgtgtgtctrgctcctccgctgacagagtctttctcataggagaaagaagagaaccaggaaaggtgatctttcctaaacacactctaccatcttagatatctttacaacaggctttcagaactcttgtgggggacatattgcactgcccaatttaattgccagtgcttactggcagtatcttaccagggcagctatggttctgcccactgtcaataaaatgcgcagagtttcagtgaagcaaaataaacaggctctagagatctgctgtaaccactgtcaacaataatatactgtacacttaaaaatgt

SEQ ID NO: 84 - DNA, 599 bp, Homo sapiens
ctggatggtggcaccctggaacatggtagatgggacttgagggcctgctgggggtgggagtctgtgaccctgaaggagtcttctcaccctgactcttcattaggacaacgtggcctagaaggaaggaccattcagcagggtcaggagcctcagctgggccggccaggctggcaggggtggagatggccccctcgcttcccaccagggactctgagtttgagacttgaggtctagcaccacttccagagccctcagaactgggacccactctaggcatggggagccaaggcacagtctgayctacccccaaatccaaggaccagtaccctcttggtctctccctacttccttgtttagaagccagtctcagaaacagagctcatcctcaacccattccaagaagtagaggatgaaacaggatttaaatttctgggaatttttatatcttacctaaaaacccttgaggcttgaatggggacattccaagcatgcagaaggccactgtgtcacttggatagtaggcccgtgagatcacctggaactgtgttttcctctctgccctacagacctgtctcctcctcttcctaggcacatggaca

SEQ ID NO: 85 - DNA, 599 bp, Homo sapiens
ccacccctattcccagtatatttccctcctgtgtcaagccacactccattttctctgacttgtttccttgaatgttttgcagaaaaaacttgtctgagatcatggatacatttctatgctcttgcctttgttccagccattgtctctttttagaacagcctgcttaactccctcctggtctccagcgggtttcttatcttttacttatcctaaaagcacctcctacagtctaacatcctttatgactccttctttttattgttgttttttgtttgtttgttttgaacaaagataggagayagcaaagcagcctgggtcttaacagaacagcttggaattccagggcctaaattgcttttgcaaacataaacacacacacagcaaccatgagaaaaatgaaaattaaaaatggcttccaattcagatgccaagaggctcagcctaaatggccttctgcataattcactttggacactttcatcctctgtaggtgaggacaacaggtctggaccaaggttaataccttaaatggttcgaaataactgactttgggcagggtgcagtgcctcatgcctgtaatcccagcactttgggaggct

SEQ ID NO: 86 - DNA, 599 bp, Homo sapiens
cctgggggtcccgaggcctgggcagaaggcactagggtgcactaacaggaatttaggtgctgccagcttgctcagcatggaggaaacctctgcagaggaggggtggactgatgaagaggagcagcagaagacaatttcacaccgagtgcttacaaagtgccaggaaccatattatctctaatataagacacgctgccaggtagttatcatcttcatcattttaaaaacaagtaaagtgaggcacagaatgttatgtaacctgtgcaaggtcacacaaccagaacatggcctctctacctycactctacatgtactaatctccatccccagggccgagaggccgcaaccggatggtgaggtttagtcttagccctgatggtggggtggggaggggacaagggtgacttgaaaagctctccgttccccttggcagctgtctttccattcgcaccccctcctgcacccctctgccaggcctgttgatcacagaggcaccatcccatctgctccgcctagaagttcagaatctcccgagatttctgaagcctccagcactgttctttgtgacactctaaacacagcagcttctcctcctgggg

SEQ ID NO: 87 - DNA, 599 bp, Homo sapiens
aatagggagagagactaagttcaactaagtagaagcaaaaggtgagaaggttttgaggcacttggttgatccagtgaaaaagaagttatggtgaagattaatcaatgggatgttttgagcatattgagtcgttcctgagtttgcaaatgcttttccttgtgattaggccatctgtgttagcaaatcagcatacccacagaaattaagctactactctcctatagaggatggaggatagaagtgttattttcttcatgtttacatttcaaagagatggttcccagccctttgagacagacrttttctgggttgtaaactggaaagaggctatttacagccatttcaaaggaacagaaaaagagtatacaattggcaagttttctacagtaaatgctgtaagtaaagggggatcaggagcctaaaggtagaagaagcctgtttaaaatgtgtaaagctgaggaaaacattgattccatgttggttagtagtcattaatctttaatatattcttggaactgtgttatttaataatctcattaaatatttagaacaattttgtaaaaaaaagatatttatctccattttacagaaaaataaaa

SEQ ID NO: 88 - DNA, 599 bp, Homo sapiens
cagtggcagccagaaggctcttttctcacagtggaactgcagggggtggggggaggggaaatgataaaagaattttcttttttaatttttgcgatttttcatccatctcaatgcatcagctgtttggcagcagcacaagtcagagacatatgtccatcggatttactcctttttcactgcttccattgaaggccaagaaattcttccactgtgacttcctcccccattccggaaaggaagtccttagaacttttgcctgtaggaccccaaatgtcgcaagcccttagaacagtggtcccyaacctttttagcaccagggacaggtttcatggaagacaatttttccactaccgtggggaggggggtatgctccacctcagatcatcaggcattagttagactctcataaggaggacaaccttgatccctcacatgcacagttcacaatagggttcgcgctcctatgagaatctaatgccgctactgatctgacaggaggcggagctcaggagagcagcctgggagttggggagccctgccttagaacttgcctataggaccccagatggagaacaggccatggattgttcctcttcacc

SEQ ID NO: 89 - DNA, 599 bp, Homo sapiens
ctgggttggggctttctgggacttctattaggtgagaatgtcccaggccagctctcccctacccctcccaacctggggtggttctaggagggtgctgggggtcaggcaggcctgggtgggaatcctggcacttctgactggctgtgggaccccatgtaaacatacaatcttatagagcctcagttttctaatctataaaatgggactaataataataatagctgctccatgagatttttcatgacattgaagttaatctgtataaagctttcgtcactggcacatactgaagttctccayagatgctgtggttgctaaatcatcctggtaagagactcctctagtacgtgctttagaaagcagctcttctggataggcgtggtggctcacgcctgtaatcccagcactttgggagtccgaagtaggcagatcacttgagctcaggagttcgagactagcctggccaacatggtgaaaccctgtctctactaaaaatacaaaaagtttagccaggtgtggtggcgtgtgcctgtagtcccagctactctagaggctgaggcaggagaatcacttgaacccaggaggtggaggttgcagtg

SEQ ID NO: 90 - DNA, 599 bp, Homo sapiens
gcagccagggccctggcacacagggcctccccgatgcttaaaccctgcccaagcttgctggaaagattcaggggagaaactcccagaggtttcaggagaggaggagcaatgatggggaagaacatggggtggtttgccaggtatcaaatgctagaaatgagggcctggaaatagaagcttctgacaaagagctacccttctgtgcatgatagattagaaacttgcaaactccaaagatgggagggcatggaattcaagagggtggggatcaataaatatcagagtctttattcagactcrgtagggaaacaacctttaaggtcaagaagtcaccagatcttaaaacatgccctggtgttcccacagtggcagggagcctgaaggtgcagggtggcagaggggggcccagggtggtggccttggccagggaatctctagatgtgaaaattgctctggcagccccaggcttgccctttgcttgggtcctgaacactcagcatattacactcagggagcatgagccgcttccttcattctgcaggggacagttgccttcccaggtacctggcctgaaatcctgagtgctgaggaagggcatg

SEQ ID NO: 91 - DNA, 599 bp, Homo sapiens
cggtggggtgagagcattgtaggtaggtaagattggctttccaccccacttctcccttcccaaactagatgcattcccccaagtattgttctcctaaaaccacttggcttcaaccaggtaccaggtgcagccaactttctatgcaatgaaaagaagccaggatcaagatgaatgcagaactaccatcaaaaatccccttgtctgcttattcttgcaagcagacataagatggcctgataaagaattctatgatttcagtcctaatgaaaacagtatgttggcccaggacccaggtactaygttttaagtctccctgatgtgctgactggcatggacgagcggaagaagagggttgatgatgccacaacacactagcagatgggtcacactacattgtgaaggtgaatggcagaagacgctcactctttggtccttgctactttggcagcaacagctcgaaatcgacgctaagagttcctggtacaagcagactctgctagtcccatctcctctaggagctcattaatttactccaaatagccagactttctttttcatttactggtttccgaatcaggagaaaacacataaaggtagag

SEQ ID NO: 92 - DNA, 599 bp, Homo sapiens
agttagagtaggaaagcaagtcacttatcctcttcaagctttattttcctcatctgtaagaaactacctgcctcgaaggataatttcaataatatacgaaaagtccttaacacagcttggagcatagcaacaatttaaagtaaatgttagattgaatcatgatattgaacagctaggaaaacccttctctgaaattcagaagtaaaggcaattttgaatagaacacaaccaacctatttcatttagcttcaaaatatttttgtgatatatcacatacctttctcctgaagatgaattaayaactgaccaattctctggagctgaataggaataaaaaagtcacagtatcaatgagaaaagctcaggatgaacaagttttatattaaaatgtgaaattgtataatacttaccaagtttcctgcttttggggcaactgtcttccgtctattttaatggaaaaaaaattgttagaggagtaatcggagtatcaaaattcaaaaattttacaaattattcttatgtctgattttgaaaactaggaaaaggggaaatcaaccatttattcttacctcctggacatctgaactacccttttcagg

SEQ ID NO: 93 - DNA, 599 bp, Homo sapiens
aaaatttatgaagtatatttattttttcttgctgctataacaaattaccacaaagctagtggttttgtatagcacgaatttattatcttactgatctgtaactcagaagttccaagttctgtaactttccaatgcaggtttcactgagctaaaatgaaggcactggcagtgctgcatttccttgtggaggctttagggaagagtcagtttccttgcctcttctagcttctagaggctacctgcattctttgactcacggccctttcctccattttcaaaatcagaaacattgcatctctrtgaccattcttccatactaaaagatccttctgggcatcacagcatggaaaggttatttgattttaaatacccttgtaattagattggactctccccaggtagcctatgacaatctcccatctgtcaggtcccttttgccattcaaagtaacatacacaagtttcagggattaagacatggacatctttggccatcgcattctgcctcccacaagtaaagttgacatcaaaagcacgttcaataaaaagaaacattgaaataaaattaaatcttttgctctgtcaaagacactagtaaga

SEQ ID NO: 94 - DNA, 599 bp, Homo sapiens
taaatacttatagaaactatgcttttgaaatgatttttttaaagttattttctttgaccattagttgaagcctactagcaacacacaatgtttggagccaaatggttaggcagggcaaaaccagggggagcctaatctttggtcctctggtggccacagcacctgagaaactgaactgtggcttcaggaaatccactctggggccacagaagaaagaacagttgtaccaaccaaggcctggcctctggggaccagtacagcccatagcagaaggcaagatgtctgacacaatcctggcaygttccttaaactcatgggagggctggtaaccactggaccagaacagaagctcctggaggcatctgagacttgtgctcagctctttccttccttggcttctcttgctaagaagctcagactggcactgcaaaccatcaatctctggggcgaaatggcaaggtcatcagatatgcatttcttaggatagaaatgcaggaaggtaaaacttcatccaaaacagataacagtttaagagaaagatatgggctccacccccaaattctgggagccctgggcttaggagatgggctggggaaagg

SEQ ID NO: 95 - DNA, 599 bp, Homo sapiens
tttttctgtcattatttctttgtatctcagaaatagcatgtagctgcattttagggatttcttttcctcctgatttgttgtttttcatgagacaattcaaaccattcccatttattgtgattactgacatgtcaaagcttgtttctgccatattgttttatatttattaatatatgtaccatgctttctacttttccccacggttttccttttttatcaattgaccagctcttcttcatttaatttctgattaagtgcttacctttaaattttaactccaacatctgtccattattttaytttcccttatagcctgtgtaagtgtagtgccccttgtcattatgctcggcaaagcacaaaccttagcccacttggacttccttcctctccttctcctccccacctgcaatgttaataccacttgagcttttagttccagttccatttagacttagcagcaaattctaattcctttactcaccactgcttcttgaatctgtagctttctcttggatttattttcattgtgttgaagtacctcctcctacaggtaggatggaaggtgactcattttctcatctgtggatgtctgaaattgt

SEQ ID NO: 96 - DNA, 599 bp, Homo sapiens
cagaaaggagtaagatgatatattcaagacctagtagggggaagccaactaagaatactatacccagaaatcatgtctttcaaaaatgaaggattgaaaaaacttcgtcagacaagcaaaagttgagagtttatcaccactagacttgcattgtaaaaatttcaaaggtagttcttcaagatggagaaaaatggtaattagtaacatgaaaatataaatcttactgataaaagtaagactgttttaaaatccagtacactctaacactgtaatgacaatgggtaagtcaatcatgtcttyagtataaaggttaaaggtcaaaacgattaaaaatgactaaagctacaataatttcttaagggatacaaatgctgaaaagatgtaaattgtgacatcaaaaacataaaacataggaggaagaggattaaaactgtagagcttatatatgtgattaaaatcacattgttatcagtattattcacaatacccaagatatgaaatcagcctaaatgtccatcaacagatgaatgggtaagaaaggatgctgtatatattcacaatggaatattattcagtcataaaatataatgaaatccagt

SEQ ID NO: 97 - DNA, 599 bp, Homo sapiens [sequence omitted]

SEQ ID NO: 98 - DNA, 599 bp, Homo sapiens
gagtgcatgagacagagaccttttaagcatgtccttccttccccaggttgcaactgactttctccagggaaggaatatattcccaagttgatcaaatcgtgtggatgcaagtcttccaatgtgctcagtcaacaaaaatgtctgagcctgggatgtcaggtaacccacttttcagaatacttaaatcacacaaaggagcattctgtttagtactgggaccacaatgcttaagctgagccttaagaagtcagtaagaatcttaataagatttggacatgatgcccacagccctgatactaygtccaggcagcttaaaattgcttgaggttaaacatttaagttttatacccagccaactttctcacaaaggagtggaggtgatccacactgacatatgtataaaccatacatttaggatttaacatatgttaaggttataatattaatccgaaattaagaaaaacagaacacaaacgctataggaaaacataaacatatgaacaaagactagaagaaaacaaaaacctaagtagttgttttaggaggatgggattgtaagtaaactcgctttctcaaaaaggaatctgaatgtcatttta

SEQ ID NO: 99 - DNA, 599 bp, Homo sapiens
ggcagacggggagtagcagctgcccctctctggatcaggcctgagttctctggttctctataatctctaccactccctaatgtctcatacactggcctgcttgtctatttgttacctccctggatcctgcagcccctttagtcagcctgtttaatccattctgcttggcaactgaggagaagaaggaaattatagacaggttgtatgtgggtgtccatgtgttaaaatccttctgaaaactcctgatctggctgctctcctttccctatgaccaactttccacccagccttgccacatcrgacctagtagactcatcaccaagatgtgatgagcctagcatcctactcccaaatgaaattcctggttatccacacagggccctcacttttgagtcccatatgctgaagaccaggattacagtggatgccaggtcagtttcatgtactgtgcccctctccattccttccttattcagcacatatatctgagtggttactgtgtgcctggcactgctctaggcattgcacttgtcctcatggattttacactctatgggggaaagtcagaaaacaaataatatgtaatacataagatgctg

SEQ ID NO: 100 - DNA, 599 bp, Homo sapiens
gcatgctaagcactaggggcgtcccagtcctggttgcaactagctgcgacccctgttcttccagggactctttctcacctcttttcattcccttcttttatactcttagctaaatatagagtgacttcttgattaattcatctcagttcaactaacctctactggaaccctactctttactggaaggagaagattcctgcaagggattcacacatgagtaagatgtggtccctaacttgtcatattccgccttagaaggtattgcaatttaatgacataaagcattaaaatttccacaakaggaatacaaagttctgtgggaagacagaggcaggagagagtgattcttgccagggaactagaagttatcagagaggggcagcactggagcttgtctgggtcccaaaggatggatggaaggatgcaaggaggtcattgcatccaagggacagagagaaaggcatgcaggtgcgaggagctttgcggtgtttaaggcactaggaacagcctcataggcctcaaggggtgtgtgatgaagaatcggaaataagacctagatggtgggctggaccaggtggtagagggatttgaatgctttg

SEQ ID NO: 101 - DNA, 599 bp, Homo sapiens
tacatctatgatataatccaaacctgtgagtataatgttaaatgcaagtagaacattttctctgattatattattttaggctgaattaaaacttgcaaatgtgttaaggatgaataatgatggcttaatatatcacacccccaaggcatatgcacttcacaggggcctggggcccaactggcagcagaagggaagagggataaaagtggaggcacaggccacgtgcacctcatccatcacccatggttattaattgtctcctcagctgccctgacaaggctcttcctggggctttctgcrggaggcttgcaaggtgaaagggaaggtttttatttttattgctgcagaattgaggatgattctttaaagatgacgcattaaatcaggagagacacattaacaaggggaggtccggagtccaccttgcacccctgggcagctctggccctgcaggcattcactctctctgcactccctcctccgccaccaggtagaggtgctaacactcactccctgacaggctgtgagtaaacaggtgactcaatctttaacccttgctcagtgggttaagaaattagttaattattcacatcccactc

SEQ ID NO: 102 - DNA, 599 bp, Homo sapiens
gagcagccttggcttccggctcatgcactcacgatgctggctgcgaatgggaagtccctaaacagaactgagccccctcattcagctcagagcaaacggggctggcagcagcccagggcaccacgaagcagagtgcctctgtgtgcccaacacagccgcagtggaggtcaatggatgtccttgccggcgccctgctttttgcttttttaagaaaataagaagcctgcagctgcggcaaggagaaagaccccgtgggtcgctccttttagactacgctattacacctgctgactatccaayagaaaaaagaaggagaaggaaaatcgggttaactttctggtatttagtttgcagatatgtcagcaggccaccttctctacttggcaggatcacgatgtattaagaatccttttgtccaccaggaaagacaaatttcctgtttgtcttgcatttagatgcaacaacatcatcctaaagagatgcccctgatgtgtgagagcagaaaggggtgatccctttcttccttatcctaaggctcacggccaacacccctataacaaaagacaggttaacaagagaaaagcatgacaaatttattt

SEQ ID NO: 103 - DNA, 599 bp, Homo sapiens
aagtacctgatgtctggcttgccatgggataggtaggattcatccatcctaacttcccaccctaactgcccccgtgtctttttgtgcagggggcccagaaggttagtctgggcacaactggccttcctcagtgccgtagaaagggtggtacactggggcaatatttatttacattgcagagaaacctgataagaggttgagaaatacttctgacattaattataatatattaccaggttatatggattaaatgatcagattacactccagtcatgcttctgaatatgtaagcacccccayaaaagaagtgacaatttcccaagatacctggactcaaaccgtagggtgtctacccctcctctctgtggtttctttccctgagttgctcactgcccatgtggcttctctgtagggtctgtcaccttatgggattgattttatggcagctgtgctgtgactcgtgcatccacagaacaatgttgaatgactgtgtaaacgcattgctgtaagcaacctgaggtctgaacttctccttatctgattgttccctctaagtaatttgtagcaaatttcaacattgtttcccattggccagcaaa

SEQ ID NO: 104 - DNA, 599 bp, Homo sapiens
ggaggccaaggtgggaggatcccttgagcccagtagttcaagaccagcctgggcaacacagtgagaccctgttcctaaaaaaatttaaaaaattaaaaaaataaaaacatgccacaggaccactgctcagagctctttggtctggcaaccctggaccttcacatggttttgtttgccaaagcaaagtccttattaatatccaaatacagtttgagttttcttgggctttatcctctccagaaacccaggagtgtgtcaacttcttggttgcattgctttcttcaaagagagagtgaatcyggtagagatagaaaggatgtcctttacgtatcccacagcggcctcacggagaacgataagagaacaatcattaaaactttcattgccataattttctgctttcgttttttaaaagtttgggttagcaaccaatttcctccttgaccaggtgattttttccttagactgtttatccagagcattaaggaaaacagagcataccaccctcccaccatagctgataaggggattcaacacaggtgcgtgtgctagagttaagggaacatattatgctaacatgaatctagactgacttaaaa

SEQ ID NO: 105 - DNA, 599 bp, Homo sapiens
ccatacactcagcaagagggccatggaaacctggctattaaaaggactcccccatggagcacagccctgtggaaatctgagggtgcattaaagtaaaacaagtcaatgctcatcagaactcccttccagattccaaaggtgactggagtcaacaagcagttatccctgtgtcctcccctgatgtgggcagctgggtccatgaaataagtgggtagctgcagcagtgcagagatgggctgaatctagacatgttctgtatagacgctgactggtctgggctttgcttacccggtggaggaygcaaatgctcactgtgccattcaaaagccagaacagaagatattgcaccaatttggatggcccagtcacatttctttagaccaaggaacacactttacagcctgtaatatccaacaatgggcagagctagtctcctccgggtaatagtttgatagaaaagtagaacaggcaattcaaacattagttgtcctaaacggggggagatgaagacacgaagggctggcttgcacatcttcacaagtgtagtgcacactcaccatgagtggggctaaaggcgtgttcccctaacactttcctcc

SEQ ID NO: 106 - DNA, 599 bp, Homo sapiens
caaatgctcactgtgccattcaaaagccagaacagaagatattgcaccaatttggatggcccagtcacatttctttagaccaaggaacacactttacagcctgtaatatccaacaatgggcagagctagtctcctccgggtaatagtttgatagaaaagtagaacaggcaattcaaacattagttgtcctaaacggggggagatgaagacacgaagggctggcttgcacatcttcacaagtgtagtgcacactcaccatgagtggggctaaaggcgtgttcccctaacactttcctccgkttcctggtggatctggggtagaggggtgggaggatgctggtatgactatgcagttcttgccaagggagaagtacccaggcataatgactgcaattttttttccttccctctccacatcacgttaactttttttcccctacctgatgcaggggtcataggcccagggctgcaattataaatgctgacagcagggatgattcctaagcaggaaattgtcactatgtttttaaaacttgtgtcagaattcccaagggcctgatggggatgggttgtgccttcaccccatctggcaaactggg

SEQ ID NO: 107 - DNA, 599 bp, Homo sapiens
gcccttataaagagacttgagtgagcttcctcgcccttgctgccatgtgagaaggcagcaacaggacaccatctatgaaacctcacaagacccttaatctgctggcatcttgaccttggacttttgagccttctgaactgcgagcaatacatttctgttgtttatgaattggtcagtttaaggtattttgttacagcagctcaaacggattaagacagagcccaaccaaacacccgctccttggatcgctcccctccttccttttgttgcctctcccctcattaaggaacgggaagggaragatgagggataaacaagcgtggtgacggattggtcctagactgatgcggatgacgtatgcatcagagatttcctgggccttgctctgactttggtcttccagcagtgcgcaggcctgctctgccccgctctgccccgctctgctggcctcagccttcctcattcatcattgttcacctcactcccatccaccttcttccctttttcccaaacctatcctgatgagaagtttgaattctgttttaaggaaagaccttccaatgcagtgtggagtgagggccattcccacatacatacac

SEQ ID NO: 108 - DNA, 599 bp, Homo sapiens
tgtgctgacatttttgaaagccctaaaataatttggtgcccatagtgggaaccttgtcatacttatttacatagtatggaaggctatttgacaaaaaaatcaaatgaggtataggaatagtaactcattataaattggggacaataaagccaaattccactggattttaaagttatacaaatgattctctagtccctaaattagggatataaaagtccgtaggcagctttggggctgaaaggggttctgtaagcatcttgctgtataatggagcaagtcctgacccagaaaagaatctgyactttgtcccagatgggctataacttgctctgtgactgccatcatgtgtgttctggcagagattcataggcaggcagaggagtggggaagcttcatagcagaaaagggaaggtgtcaggtgtgtgctgactggaggctgctggccggggaagctggaacgaagtgggctatccagaggtggtgtatccatcctctgtagttggttagggcacttagttcactctctccagttggggttgattgtagagttctaactttttacgtggtctggccattgtccatgtgtatattcagtctct

SEQ ID NO: 109 - DNA, 599 bp, Homo sapiens
aacgggtgggtgagttatgtgctggcaaaggcaggtgtgtccacatgcgcgagcagacactgagactgcagcccacacacccagaggaacaagcaggacatcttaggttaggaatgtgcctgcagctggcagtgcccaggagagggacttttctaactgtgttgtccagtgccttagtcgctaaccacatgtggctgcataaactttaaaccagttagaatgacataaaactgcaaatgcagtttcccacttgcactagccccattctaagtctccatagccacctgtgcccactcgccrctgctccaggagtacagattccagacatttcagccactgcagaactgtctcttgggtggtgtctgcaggtgcctgctgcagctctgccaggcaaagtagagtggctgcttggctgcctatggatttagatgatcctgccaacatctgatcagttgctctcagcacctgagcttcatcattaggatggatctggggcatgcagggagaatcccagactgtcagagaggtggcctggcctggggtgaccagtcaggcctcttgtccttatggtcacattctcagaattggggccagggatc

SEQ ID NO: 110 - DNA, 599 bp, Homo sapiens
taatttctttttaacaacaaagtcttcatttttaagacattttttaaaagtaagtcactatggattttatagatttctgtggtttaagtaaaatgtgtttcaacatagcataaaacctgaagaagaaaaatacagagttcaactattatacatacttgtactatatgaaagtcatataccatttgaagtacgtaactatcagccctaaagaagccactaaaatagcaaaagcctctatctaataagtcaacaaagcaaattaaatgaggtattaaaaaaattaattcaaaagaagaaagracataaggaaaaaggaaaaaaaacaggcaaaacaaatagaaaacaaatatcaaaatggtagactttaatctaactatataattacattaaatctaattaaatctaaataaagctaaataaatctaaataaaaaatctaatagatctaaataaagctaataaagtaaaaaggcaaacattgtcagagtgtataaataaagcaaaactcatttaaatgccgtttataagaaatgtactttaaatataaagaaacaagcaggttaaaatgaaaaggatggaaaaagatatgacatgctaaca

SEQ ID NO: 111 - DNA, 599 bp, Homo sapiens
caggccattttcacttcttttgtggtggaatgtcaccagttaaggcgggaaccggtcatctggatgtgtacatgcaggtcacaggggatatgatggcttagcttgggctcagaggcctgacataaagtaataaaacttctaaaaaaaaatacttagcacgatgaggtcctgaggcacggaaatatttagacattttaaatagatattcaacaatgctgtaagtaactggtagaatttccataccaggtctcttcctatgtcactagcccatgggcacaagaggtcatgaaagaggaacaraaataggaaaacctgcatcaacacagaaacagcctgttctttgccagaatggagaacaaggcaaagctggtacttttagaaagctgaaaatttgtgccaatgatttttttcctagcttatttttggctttttgattctaaggagttttgacctacaaagatgctcaatatttaaatagacagatatttttgttttatttcttccattgcttttatacttcggcatcctccttactgagatgagataaagttttcaaaaacattgtatggaattattttctatgtatcattatttaattc

SEQ ID NO: 112 - DNA, 599 bp, Homo sapiens
ggcacggaaatatttagacattttaaatagatattcaacaatgctgtaagtaactggtagaatttccataccaggtctcttcctatgtcactagcccatgggcacaagaggtcatgaaagaggaacagaaataggaaaacctgcatcaacacagaaacagcctgttctttgccagaatggagaacaaggcaaagctggtacttttagaaagctgaaaatttgtgccaatgatttttttcctagcttatttttggctttttgattctaaggagttttgacctacaaagatgctcaatattyaaatagacagatatttttgttttatttcttccattgcttttatacttcggcatcctccttactgagatgagataaagttttcaaaaacattgtatggaattattttctatgtatcattatttaattcatatggaatttattaagggcatgagataaaaaataatatttcttccaaaaggaaagacaactctctcagaccattacttaaaaaaatccccattgatttgtgaagccacttttataaaacatgtccttttggtgtatactatgatttgttgatgggttctcagctctgttct

SEQ ID NO: 113 - DNA, 599 bp, Homo sapiens
agggacagagttatttccgactggttgcagatggggagaaaggtctatggtatctgttgctgatctctgggctcacatcagacactttgcaaatctggctctctccaggggatttgggtaacagccctgcctttcagagcaaataacagtatacactcgcagagctcgttatagcagttttggtgggtttagggacgcaagttcccagccgggactattcctgtggagttccctgagttacaccctccaggccacgccccctggccaaacgtgagtctagagcagagagtttccatgtaraggcttcatctgtttgtattggctcattacagacactccttgagatttgaccaatgcaacatgtggccccatgctagcgtgctatgtgctgagacaacagcttagagcaacagctggggaaccttcccttctgcctctgttctgaaataaagcacgtccatttaagacaaagggggctatgctgaaatgacttatggggaaaattacaggtcacaaaatgtaatgtgactataacatattggaatgttgatgactgctcgccataaagtggtcaccaatgtttctttcaccccccaaca

SEQ ID NO: 114 - DNA, 599 bp, Homo sapiens
taataaaaaaaagtaaggtgattgatatgttaattagcttgattttagtaatcatgtcacgatatatatgtgtctcatcatgttgtgcactttaaatatatacaacttttgtcatttctatcttaatagagctgtaaaaaataaaaatatgaagcaagggacagtgttcaccagctcataatggatagaaagtataccttatatttatttttatgctagaggtttattcagttattcactcatgcctttactgaaaactaccctcttctaggcatagtgctgggaactgagcacacaccratgaatatgtcacagcccctgtccttggagaccttcacctcatggtctagcaacaggtgaacaaaaaaaaaagcaatatgggcattcagtaaatccctgactggtatgcaaacatgaggccagtgcccccactggatacccctccctagtaatgtagaatagtgaccttttcaccacaccctggctaaagcttctagaaacacacacacacacacacacacacacatttagaggtacagactgtgactatggaacaattcagtgtaacggccacatgaggaaccgatgtttgaccttaa

SEQ ID NO: 115 - DNA, 599 bp, Homo sapiens
tctacctaatactctgtaactatctccaaaaagttaaataaatcagatcattaactagagaggcctttgaggctaagtgacttgcccgagtttgcaaatcttgttcagagaagaacagggaccagttcccagcacctggctcttcctgccaggattgatgatattgaccccactgccctcttgtcatggttcctgaaaaaaactcctctgattaatgcaccaccttaaaccctactagatgatttcagaatccaaccagatgacacctcttagataaagggctctcttagagatcggctrtcggggcaatcgttttctagaaatagcctctgcccagaatagatggaagtctttcaagctggctattttctaaggtagaccctttctgaggcctttgaagttactttgcagctgcaaatggctgccctatggacagaataggactagatgtctaataattccagagcaggcctgcacagttatctcaaagcacagtgccccaagtgaacattttcttagcataaacatcctattccttaatatgttattacttccagcaaacatagatttcttaagttctttcaatttgagataaagtg

SEQ ID NO: 116 - DNA, 599 bp, Homo sapiens
ctgagcccagttagaaagtcagcacggcacacaagcatttgggaagtttactttagattcttccacagatatcagtgtgtgagttaaccaattttaaattatcctctatttaacatgttcttattacaaatatttgtatcaaaaattccaatcagatgtactttgcttcactacctttagtctacaattattgatcttaatcactttgatgccagtcttcaaccttggtggtcagcacatgacttctgggttttatgaatggccataaaaccagacacagcgtgtccagctctgtgtgtyaggaaccaagataaatcatgatcttcgcagggttcagttggctcttctgttaaatgaggggacagaccagattcgtttgccttcttagtttcataattctacaattttaattctattcaagttttaaagcttctagagcttatagtataaattcaggatctaaagctaataacaaagtataaaataaagatctcttgttaccacttccctatcttcagtgaccccttgcaaagaaaagacacatctttttattttcagagatctcactaaagtgcactgtaaactaaagagcccatgaa

SEQ ID NO: 117 - DNA, 599 bp, Homo sapiens
tgatgtttgtgagggaaagcatatgttaatcagcttgatttagccattccacaatgtgtacatatgttaaaacagcatgttgtacatgataattatatacagtttgtatttgccaattaaaataagatttttttaaaaagtgtaactcaaatgtacctgttagcaaagagctgagaggccctggaggggtcctgggaagcaagagacctgggccaggagcccaaagccccccacccccatacacattcctgccatcaatcccttgtgttacattggacaaatcactttttccctgagccygttttctcctgtctaaaagagagatgccacctgtaccctcacttccctcccccgccttccttccatcctttctttcttcatccaagcattagttgagctcctggcaaatactggcctggtggcccagacatgagttactctgtcttctggatagcttccacacccccaccaactttattggatcattgtgggggaataaatgataagacagaggtgatgctgttctttttaagaaggtacttcccaggcatgaggcatgattgatctttttacagcattcctgatctaaaggcaggatt

SEQ ID NO: 118 - DNA, 599 bp, Homo sapiens
aagacaacaatcctattttaaatggctaccaaataattggactaaatttaaccaaggaggtaaaagatctgtatgctgaaaaccataaaacgttgattaaagaagttgaggaagacataaataaatggaaagatatctcatgtttatggattagaataattaatattaatatcaatttatgaataaataaaggtttgtcacctttattgcactgaggtacattcatttatactaaatttgttaagaatgtttatactgaaagttaaaatgtttatactacccaaagccgtctattgactygatgcaattcctatcaaaattccagtaaaatttttcacagaaatagaaaaaaaatcctaaaatccatatggaaagcaatagaccccaaatagccaaagtaatattgaggggaaaaaagccagagacattatatactacgtgacttcaaaatatactataaagctataatagtcaagacagtattacactggcataaaaagagacaatgaccagtgaaagagaatagcaagcacagaaataaatccatgaagttagtcagttgttttttttttttttttttttttttttttgagacagag

SEQ ID NO: 119 - DNA, 599 bp, Homo sapiens
tcacctgaggtaaggagttcaagaccaacctagccaacgtggtgaaactgcatttccactaaaaatacaaaaattagccaggcatggtggcaggcacctgtaatcccagctacttgggaggctgaggcaggagaattgcttgaacctgagaggcagaggttgtagtgagccagtgcactccagcctgggtgacagagtgagactcatctcaaaaagcaaacaaacaaaaagtgaaaaataaaaataaatatttattttaaaaaagaagagagatctcaaataaacaagctaacatttatwtctcaaaaaactagaaaaaggagaacagactaacccaaaagttagtagagaaaaggaaataataaagatcacagcagaaacagaagaacaagaaactagaaaaatgataaaaaagagaaacaaaactaagaaataatttttaaaaagataattaaaatggacaaactttatctaaactaagaaaaacagaatactcaaacaaaatcataaatgaaagagaaaacactataatagatactgaagaagtacaaatgatcataagagactactttgaacaattatataccaacaaattgtat

SEQ ID NO: 120 - DNA, 599 bp, Homo sapiens
tggctgagtggaactcttgtctctcagtattatactaccttcatcttctctgttaagtgcacagcttaacagaactcctacttagacaggcctcatagaacctcattctgttcagaagcaagtgtttctcagccaaagattccatgatctatgaagatttttacaggcctatgccagtagatcccagtgctgggccagccccccagacctcatcccttgtggacctaagtttcaaaccagcctctacagcccgaggatctaggttagccctcatgaacccaccctccaggctggccccartggaccaatacaccaggcacaccccaatgtatccaagccccaggaccatctcagtgcccagaaagcccctgcagactaaagcttaaggcccactccagtgccaggtcagctcttgtggacccaggcttcaggccattccctccagggctgcccttctggacccaggctccaggcccaccccatagacccagtcaacttgtccccccagtggatccaggatgtagacccaccacatgggcccaggcaccaggtcagcctgcctgaagattccagcagtaaagtttcttacaaactatacc

SEQ ID NO: 121 - DNA, 599 bp, Homo sapiens
gtctgtcattattcttctagttgttcctgtagggattgcaatataaatccttatcactgtctacttagaattaaagttgtgccacttctcataaaatataaggactttgaagtattatagttccacttattaccatccttagttatattattgtcatatttatcatatctaaatacattataaatccccaatacagtattgtctttttaaaaattaagagaaataaaaaactgtgtaatcttcatatgtgccatatacttactatttctgatgctccttgcttgttcctgtacttctacwattacatttggagtcgatttttttcagcctgaataactttttttgctattccttataatgtgaatctgctggtgactgattctattttcattcacctgaaaaagtctgtatttcatcatcatttttgaatgaaatttttgctaggtacagaattctgggttgaaggttgtttttattcccctgaactttaaagaagtttttttcaaagtattctccctccattaattctgatgtgaatcattctgatgtgagtcattattcatgttgttgcttccctgtagttaatgtggtttctcact

SEQ ID NO: 122 - DNA, 599 bp, Homo sapiens
atctccatctgccatctattacctccatctaccaactccacctaccaccagtgatgaggtcctcattgccagctgaataagtgagtgtgccagctccaaaactctattgtttcacctgctttcttagtacattcctgctattaactgttacagattgagttctctgggaaagtctctgagaaagaattttgcataagtgcttgttcaagagtgtccttgagattaatgcctgaaggagggagaaaggaaaaggaaggagaatggggcagagagagaagttgagctgtaacaggggcccamcagtaacacctgaagacccacagagacctctggagctacagtggcttttcagagttgacctcagttgggccaaaatggtcagacattatattttttttattgttctctcatttaatgtgggctgcccaaggaaggagtgggcaagacgcctttctgaagctaacataattactgaagggtctgacagtagaagacatcagccaacaacagtccctgctgctaggtcaacaagtctttccttagagggatctagaaggcacaactcagtgttcatcagttccttcactggattttcagct

SEQ ID NO: 123 - DNA, 599 bp, Homo sapiens
ttgtaatggtgaattttatctatgttaaacaacaaatttaaaaaaaatatttggaggaacattgacccgcttgccccactccatgcgtcccattgagcaccctgactttggtataacagacctgccttgattgggcatttagcattctttaaagcttggctactctgaattaagtctgaggtcttatcctcagctctgtcttttctcatgctgcagatgggaaagtctttgtatgctaccaaggtttccacttgcttgttaatcaatctcaagtctttgttatacttttctttgaaatgmctgatgtatcatgtcatccacatttcagcacatctcttcattatactatctcagtttcccactagagaagtgaagccattagacttcacttcttagtcttatactagaagctatctccatctgccatctattacctccatctaccaactccacctaccaccagtgatgaggtcctcattgccagctgaataagtgagtgtgccagctccaaaactctattgtttcacctgctttcttagtacattcctgctattaactgttacagattgagttctctgggaaagtctctgagaaagaat

SEQ ID NO: 124 - DNA, 599 bp, Homo sapiens
actggcatatggataaatgtgtagaacaatggactagcaccgaaagtccagaaataagtttatatgcctatggtcaattgatttttgtcaagggttccaagaccatttgaagatggaaataataatcctttcaacgaatgatgctgaatatctacatgcaaaagaataaatgtggatccctgctttacaccgtatacaaaaatcaatgcaaagtgggtcaaagacccaaatattagagctgaaactataaattcttagaagaaaacataggtacaagttttagtgacctcacacatgcarcaacatgcatgaacctataaaacattatgctaagtgatagaagccagacacaaaatgccacattttgtatgacttcatttctataggatatccataatagaaaaatctgtagagacagaaagtaaataggcaatttccagaacgtgggagaaggagggaatggggaggtacatggtttctttggggtatgatggaaatattctggaattagataatgatatggttgcacattttgtatataataaaaaccactaaattgaagaatttgacattgtaatggtgaattttatctatgttaa

SEQ ID NO: 125 - DNA, 599 bp, Homo sapiens
atacaagatcagcatgcaaatatcagttgtatttctatacagtagcgatgaacaatccaaagataaagttaataaaaccattcagtttataatagcaacaaaaagaatatttgggaataaatttgaccaaggaagtgcaagtgttgtacactgaagactacaaaacattattgaaaaataataaagacctaaataaatggaaagatgtgtaatttcatggattggaaaacttaataatatcaagatggcaatatatgccaaaccgatcaacagattccatgcaattcctgccaaaattcyagtggctttttttttttcagaaatggacaaattgattctaaaatgaacaagtgacaccaaatagccaagacaatgtttaaaaaggaagaaaattggaggactcacactttccagtttcaaaacttactacaaatttacagtatttgaaacaatgtgatactggcatatggataaatgtgtagaacaatggactagcaccgaaagtccagaaataagtttatatgcctatggtcaattgatttttgtcaagggttccaagaccatttgaagatggaaataataatcctttcaacgaatga

SEQ ID NO: 126 - DNA, 599 bp, Homo sapiens
ttattgtgtgaacttgctgtgatgttgcaggattcttcctcctggagcccagatacttttcaaaagatgaatgtcatttctggtaaaaataaaataaaataaaaatggcattgattcagtaactgctcaaggaatcataacactgtacgagggcaattgctgtcaacctcctgatcatctatccttgttttgttctttgttttgttttgttttttgtacagattgagtagtaaccttgttggcagctcatttattttgcccctaaacatgccataatctattaggggtctaactctcagrtaatcaggtccccgactgtccctacaaccttgccgtttactgtggcaactgcatgtaccaatgcattccttgtggcctggaaatagtgttctcttgaagtttcccatgaaacacaggcatttggtgggtttcccagtcttacaaagtctatgccctcccgcaggctcacttctctgagccttttaatcctttttcccactggcaaccatggtaattctggaatttcaacctcacttggcatggaccattactgtttctaggctcctaggagccatcccagtagtgagttcacactgtat

SEQ ID NO: 127 - DNA, 599 bp, Homo sapiens
caacttcgacccagtgtccaacaatccttgacatgtttggtattctcctttccgtagtgtacaattacccaagtaactggccataaatccctttgcagaaagactggggtaattattattgtgtgaacttgctgtgatgttgcaggattcttcctcctggagcccagatacttttcaaaagatgaatgtcatttctggtaaaaataaaataaaataaaaatggcattgattcagtaactgctcaaggaatcataacactgtacgagggcaattgctgtcaacctcctgatcatctatccktgttttgttctttgttttgttttgttttttgtacagattgagtagtaaccttgttggcagctcatttattttgcccctaaacatgccataatctattaggggtctaactctcaggtaatcaggtccccgactgtccctacaaccttgccgtttactgtggcaactgcatgtaccaatgcattccttgtggcctggaaatagtgttctcttgaagtttcccatgaaacacaggcatttggtgggtttcccagtcttacaaagtctatgccctcccgcaggctcacttctctgagcctttt

SEQ ID NO: 128 - DNA, 599 bp, Homo sapiens
atttcttgtcatccagccccattctcacgataccactgataaaatgaatcaaaggccaggacattcaaacagtccagatctctttggactatattgtaacagagggtgggtaggacaaagaactaacaggtctgggacacactgcaaatttacactgttaaccatttcagttaaaatttacactattaaccatttcatgtgagtgcaaatggtttctcatcctcttttctgcaaaggatgtgaaaaaacgtatttgccaaatcaatagccacatgccatgcccctaaggctgtgttgacytcttctaaatatatcatattctgcagggtgagggcaaggcttgctactacttgcttgagttggtggtagtctactgtcatcctccagggttcacctgggttttgcagagcccagcctggtgagttaaataaagatatgatgagaaccactgtactgcactttttagatctttaaggaggggattaatttccatcatcctcctcaggggatacaatattgatttttatatactatctctgctgaggcaagggtgggagggaaccatttcaggagctttcctttgggctcctacccaatag

SEQ ID NO: 129 - DNA, 599 bp, Homo sapiens
agaaaaccacctggtatgagacaaccacctggacatgaggcagccacctggacatgagatagccacctggacacaagacacctggaaggagacagccacctggacagtagatgagagttttggaaattatttgcctgtatctttaatttcccttttttcattagaatttaaacatggcctctgctcaaagaagaaaaagaaggaggaatgaaggaaagaactgaaaagaaagaaaagaagaaggaagaaagcacatcttcctgcaactaccacttctccccttgccctcattaccccacygtgtccacttaccctgtcctcaccatgccactatcttgacacgattgttcaagacctccttctggcaaggtcattacttgtatccctggcagtgtttcatgccattgaaccttcctccttaaagaatgtgcaaccactctgtgggccactttcttcccttggcttctgaacggccccaaagcccactcctcagacatcttagtgggttctttttccctctgtccactcctctcactcgggcattccatggggttctgtccaaattgctctcctacaaactccccttgggcaatctccac

SEQ ID NO: 130 - DNA, 599 bp, Homo sapiens
tatattttgagtttatgacatgttttataaattgattatttttttctcaattaggaacatctatgcttatctaagagattccctttattttatgaggtttatattctttttggtagaaaacattttgttccaccttaactgtttgaggattaataaagtaagatgtataaatgttattattttgatttcttaattgaaatgtaatttaaagttgggtcttgctgccttttggggcttcgtgccgggcacctgaaggaaagacaaattaaattattctccaacgtgctctggatggtgagsctcaaacctgaatattttgtgttttgaaatttgagttttcagagtcagaaatttagagtatatccatacagatatcctagaataaaacatatacttcttatttaaaagtaaagtttaaagatactttatcattgaaatcatccatccatctagttgatttctacaggtgagctagcataattaaatcattttcctttcatttccattaatattggcttttctgaaaggataacaggaatctcaaattctgttatagagctgatatttaaaagttcaatttcgccatttttccaaaccca

SEQ ID NO: 131 - DNA, 599 bp, Homo sapiens
cctttcaactgtgaaatctctaaccagaccatcaccaattataaagcagtccccagccttaccatggattaagtagaaattaaagatcccaacacacatatgtattgcctggctgtcttgccaggggggtttatcctgagatcactcagcatagagccactcagtgtgcgaacacacttgcatgtggcattatagaaacaccctgaacacacatacacattgcctggctgtcctgccagggggttttatccctggagcacttagcatagagccatccactgtgcgaacacagcagcacayggcgttacagaaagatcccaaacacatacacacatcgcctggctgtcctgccatgggttttatccctggagcacatagcatagagccatccactgtgcgaacacaccagcacatggcgtataaaacatgtacaattgcaaccaccgacactcccaactgcagcgagataagctcaccagaccaacagcaacagcggcagaaagggcgctcctacctcctcatccagtgttttccccactcttcaagtaggtgattttttaaagtttatcacaaaaaaaagacaaaccttttttcctaat

SEQ ID NO: 132 - DNA, 599 bp, Homo sapiens
gtagcttgggcttcctcctagcatggtggctgcagacgtcttacaaagtggttcagagccccacaggtgtgttctataatagctcacaaggcagaagctgcattgcctttcaggacctagcctcagaagtcacactgcctcatttctgctgtattctgtcagttccaggagagtcacaagcccttcctggattcaagggaaagggaattagatgccatttcctgatgaaggtggggcaaagttttgaaagagcatgtgtgggggactcatgccatctttggatagtcccagtctccctcstcaaggacatcaactcttgtagtttgtctccagtccaaacatgtgtgggcactttccctgcatatgtgggtatccacgtgaatgtctcttcattgcttctccatctttctaaaaagtaatcacagagagtcatgaaatccagtgttagaaccaggcattgttaatgctcttatgttattattattcaagctgggctaggggaatgtggcattaataaaacaatatgtacattggatttagttatactatactctctattctaatactttgtaatcattagtaataaatagtagcatatt

SEQ ID NO: 133 - DNA, 599 bp, Homo sapiens
agaacacatggactcaggcaggggaacaacacacaccggggcctggttggcgtgggggcaggggggtggtgcggagaggggtgaggggagggcgaacaccaggacaaagagctaatgctcgtgggtcttaaaatatagatcatgggttgataggtgcagctaaccaccatggcacacgtatacctatgtaacaaacctgcacatgtatcccagaacttaaactaaactaaaatttttttttaagtgaattttagccactagattttagagttgaagcatgccatggtacttgtgattagkcatctacctgtgtatttattcacctgaacacctggatgtgtagaataaaaattgctcaattgttgaaggctgctgcatcgctctatgctacttgttctgttaaagtatgttcagggagccagcccagagaggcacctccttttagctctgtgaggccttaaagctgggtcaccatgaagtccctgtatatatattttttgttttaataactccaagtaaccaaatgaaaacaattatttataaaatgtattaagaagtaaatgttatcaatttgggactttgcacaactattattaatg

SEQ ID NO: 134 - DNA, 599 bp, Homo sapiens
atatttaaattctgtaggctagttttcatattctggaaaggtggattttcatatggcactggtgctggtgtttcatcaggtcagctgtctatgtcataagagggtgttcttattagaaaatgtaggctttggcaagctttaaaaggtacacgtgttactgcatgttctgacgagattttgaaaatctttggcaactgtgctttccaagtggagaaaaagggctaccataatgttgaaagacggaaattaaaatctaatcagatatggtggcccttatcctccaccttagaatttcttttygtggaatgtctggtggacagtaatatatagaggctcctcgacttaggatgggatcacatccagatacacccattgttaaattgaaaatatcataagttgaaaaatgcatttaatatacctaatctaccagacatcatagcttagcccagcctactttaaattgctcagaatgcttacactagcctgcagtcgggcaaaattatctaacacaaagcctattttgtaataaagggttaaatagctcatgtaatttattggatatagtatcagttgtttccccttgtgatcgcatcactgac

SEQ ID NO: 135 - DNA, 599 bp, Homo sapiens
ctagagcgaactgaaccagatatctgctaaggctctgatagagaacagctggaagctcagaaaggtaaacataattcaaatattggtgaatatgccagtgcagccgagaaggtgacaggatttgaaaaatgtgccaggacacaatgagaaaggccttggaataaatacaaaaagcccaaactttattctgtaaaagtggggagctactgaaggttttaatcaaagcagggataatctcatttccaaggcaaaaaaacaaaaagaggcattccttcctaataccacatgtaggagctgttyttattctatggtggattattactttagtagtatcttataaagacagtgcctgaaggtcttttatctgtacattgatgtaacacttttcaaaagcatatcagcagacacatttttggaagcatgcattctgaaacttgaacttcaaataggagttatggctgatatcatggagtaaggtaaaacagaagctgactcctctccagtattcaaggctgactgttgtctctggaactgagaatactgggaaaagtgtgagttggacactgtgcacaactctaattaacactgttctaaaggga

SEQ ID NO: 136 - DNA, 599 bp, Homo sapiens
tgggcatgaccattctctagtttctatctgtggcttggaatttgtgttaatgtattggttgggtcctttcaattggaatggggagagagaggaatccataaatctgtgactcagtgacctttaggaattggaagccaaggtctatttcttatgtcttattttgttattgtgcatagcagtgtctctcaactgtttgaatccaggcccctggagatgaatgtggactgtcagtggtgtggctttttccacctgcaccttctgcagagcagggtgcaccttgagcaccttgagcacgggcaycttctgcctgtgcacctgtgcacatgaaggagcctcttggggtcttgctgcctttggggcttttgctttcgcctccagctccttatacttctgcccatatatctcactctctgttttccagcctgtcttttctgttttctgaagtctgccctttccccttcatggttaggtgaaggaaggggaaatagaaggaaatatttgaagaggaaaggttgtggcactgcagggtatttgtgactttgtccccaattgcattcttcaagtgcagttcttggtttttagggtatctctttgagtga

SEQ ID NO: 137 - DNA, 599 bp, Homo sapiens
ggaagatgtctgtgctgctggtggcagcagctgctctatgcatgggatctggagggctctgtggggacccacatgtgtccactttcctgcccactctgccccactccccttccctcactctgcccgcctgggcctatacttccccttcccacagcaacctagcagaagctttgcttctccatgtgttttctgagaaatccaggctatgaaaagatatagggaaactttataagattttctcagaataaggacaaaagacttaaagataatctcactcaaagagataccctaaaaaccaakaactgcacttgaagaatgcaattggggacaaagtcacaaataccctgcagtgccacaacctttcctcttcaaatatttccttctatttccccttccttcacctaaccatgaaggggaaagggcagacttcagaaaacagaaaagacaggctggaaaacagagagtgagatatatgggcagaagtataaggagctggaggcgaaagcaaaagccccaaaggcagcaagaccccaagaggctccttcatgtgcacaggtgcacaggcagaaggtgcccgtgctcaaggtgctcaaggtgca

SEQ ID NO: 138 - DNA, 599 bp, Homo sapiens
tccgagacattatactaatgtatgatggtctccactcatgtttttggaggcctcatgggagtagtgtttataggcgtcaccttaggaaatgctgccctagacttttctgactgtttgccagccccatcaggctttggatctcgtatcttcagaaatgctgtcagtattttccaaggtaaaaactagtaaagaaaaccaaacaaaaatccagaaatgtgctcagttaatagttctcctagtatcttcccagaatagcatgtgcatttcacccttgtcctggagataatgtctattacctgkattataatctggaaatgaattctaactccttttattatgggcatgaccttgtttctgtactcccagcctccaaccagcaagaaaatgggggtctcagtcccacagccacaaggactgggattctgtaaatgccccagatgagctgggaggcagattcctccccagagcctgcagatggagttcagcctgcccctgacaccttgcgtgggagacactaacagggagaagagctgagcctcccccagccttctgacctacagcaccatgtgatcatgaatatgtgttattctaagccacta

SEQ ID NO: 139 - DNA, 599 bp, Homo sapiens
cactgcattgatttaatgcaattccatccaaaaggatgagacccacacagaatcccacccaaggccaaccacgttagacttcactatacagactctgccccatccctgctggagccagtgctgaccaggttcaacacagtctccaaggctgtcagcccaaccagcaggtcctgctgcctggatgggttttgttgctctcagccctgcagccataaacctggcactaccagggtttcccttccactaagagagaagagtgctaagccggggcatgagttaagagctgaaattgcagcctcrctctgaccatctgggtgtagtatcatcaacactgggagtacaaatggtcaagaagggaccaatctctgacctgctctaccagtgggctaggaggagagacgtgtcacagagtcaagtggacatgccacgctgcacatcagggcacagtgacagttgcctgacaccccttacttgctccagtggcagtggcaggactgaattttggagaattaccctccccactgtgaacaagaacagaaccctgtcagttaaggtgcttggccatcctttgccgtggcctcccacatggccacgttgga

SEQ ID NO: 140 - DNA, 599 bp, Homo sapiens
aagaacaagaaataacttaataggccatttgtcaatgagagtacagtgtaggaagggtggaaagtgaatataaaatctagattggggcttctggtttcctgttcagcctataaggagcttagaatttgccactcagtcttgacaacaagtaaaatgctgaacaaactgaaaaatcaataattcttctcagatccataagagaagtgagattacagggcaaactactaccttcagcatcaccccgcacccccaaccccactaaatagaaagacaggagaatacagagaatcataacacagrcgcagaaacctccttgagagagccagggtagataaacatgaactgtaattgatgaattcctggaggatcactgtggatgacctgaaggattaaaaactctagagggactcactcaaaggagggcccaagcttttgtgattttttttttttttttttttttttttgccacctgaagctctacaaggttccaaaggtgaatattaaggaaaatccctcatgctctggcagccagaggggaaaaggaacaattttgaaatatgccagaatatcgttcttaacaatgtctgccctcaggagaa

SEQ ID NO: 141 - DNA, 599 bp, Homo sapiens
aaaggcattggagaggaataagcaattgagaatatgtttcttattctcaattgatctaacagataatagtttattcaaaataatagcaacaatgtgtttggttgtgtatgtttatatatataagcatacataagcatatatagagtattttttgttataaggtactcatactactcatgaagtgatacagtgttatttgaaagtggatttgaattagttataaatgtatattgtaaactctaaagcaactactaaaaaaagattttttttaaaaaagcatgactgatatgctaaaatgargaaaatgtggaatcctataaactgctgttcaatgaaaccacaaaagccaggaaaaagtgtggaatacaaacataagaataaaaaataagggaaacaaatagaagatgataacaaatacggtagacattaatccaaccatgccaatactcactttaaatgtaagatggtctaaatgagccaactaaaagacagagattttcacagtggatcaacaaacaagacccaaccatatatgttgtctacaagaaatccactttcaatatacagacacatatacactaatagtaaatggatggaga

SEQ ID NO: 142 - DNA, 599 bp, Homo sapiens
accagacaataatttgacaccactatgctgaaccaattcacaccagtctctcagaatgttgtgcatgttaccttgttccttttcctaaactatttaataacacatatgccaatggtgaaaataaaatacaaaatttaagaacaactgacattggctgtgaactttgtagtttataaaacactttcacattaacatctcatttgatcctctctacaacctccctctagaaaaaggtgggaataaacaaaaacaagcaagcaaaaccttcgttttgaagaccttaggaattagcccaaggtyttatgactggtagatggttgaggtgagattcaaggcaaggccttctggccataacacaccattccttcttctttctgcctctctgtccttatgacttgacacataaaaaatgctagaaagtgtcacatctgtaccaccggggagactctgtgtgaaacaatagtttcaaggagggtcatcttttcagaaactgcaatcacaaaggatgcattctgtactttaaattttgtaattccagcctaggcaacatagggacaccctgtctctacaaaacatctttaaaataaaatgatataatg

SEQ ID NO: 143 - DNA, 599 bp, Homo sapiens
gtagtttataaaacactttcacattaacatctcatttgatcctctctacaacctccctctagaaaaaggtgggaataaacaaaaacaagcaagcaaaaccttcgttttgaagaccttaggaattagcccaaggtcttatgactggtagatggttgaggtgagattcaaggcaaggccttctggccataacacaccattccttcttctttctgcctctctgtccttatgacttgacacataaaaaatgctagaaagtgtcacatctgtaccaccggggagactctgtgtgaaacaatagtktcaaggagggtcatcttttcagaaactgcaatcacaaaggatgcattctgtactttaaattttgtaattccagcctaggcaacatagggacaccctgtctctacaaaacatctttaaaataaaatgatataatgtaatacagtataatatggtatgatataatattttatgttatattttcccattgtgttgaagaaaatgtttaaggtgagctcttttcctgtctttctcaaaaaggtatggcaggatcgtcatttacttgctatcctgccttggtgttggcgtcactggggttgtgg

SEQ ID NO: 144 - DNA, 599 bp, Homo sapiens
agcctgagcaaaaagagtgaaactctgtctaaaaaagaactgggttttgttggttctattgtcccctccccagcccttgaagcttggtcatgtatatttggatttctgccttcttcctctctcttatattaagtggcaagcagtaggctcttttccaaaggtttcaactgaacagatgtcactggtgcaatggtgcaagtacattaatgatgtaccttgaggtttacagatcataaaatgtgtggtaaagataaagcatgatctttttaaaccactatgattagtccatccttgtcactytttgagtgaacacatttgagctgtgtgttccttttagggaaaaataatatacccctgtttcccagcttgcactcatccattgtctatagtcctcatagccaggtcaaagatctgatcatttgtattcaattttaatcatccagtgaataatctctgctcttttgcacacatagtccagaggtaagtccaaggttttgatcttgatttaaaggtctaaatatagaaatatatttaggaatatagaatatagaagaaacaaatatagaagggaatatagaaaaataggatcatagaaaata

SEQ ID NO: 145 - DNA, 599 bp, Homo sapiens
ccgaagcagctgctgttacacatcccatgagtgtacagtttagcactgctcccacagacccactgacccagctttattcatgagcaccgtagagatcatgacaacaggtaagtagttcaatgttccacatggctgtgcttatgagagatcatgaactatgggatccagtggcttccaagtcattttaattttaatatggaaatttccaaacatactcaaaaatacaaaaatagttgaatgaatctctatgcatatccctcagctccaacagttaccaactcctgatcaatcttagttttyctatactctgcctagtttccctatccactgagtaggaatcaatccagccatcatagcatttcatcttgtaatattttcctctaaagtaggaacacataggtatctctggaactatgtgtgcactatcttcaggacactgctgaaggaggagcaagaactattcctgtggttcttaaaattcaatacaagtaatcacacctttgatccacctgtggggactgatccctcttgtgggagcaaagatggggccatggtatataaaatataaatataaaactaatatcaatgtcattgtccta

SEQ ID NO: 146 - DNA, 599 bp, Homo sapiens
caggaagtatttccatcccttggaaatgagggaaaatgtgaatctctccaaacagtaatttaatctaccacaatcagatacccctgcatcaccaaagaatgtgttgaaccctactctggcagttgaactagtaatatgaccaattgcattagctagatttggctgcaagaacagatacccccaaatctctgtagtacatgcagacacattgagcagtagtttactggggaaacagctttttcttggccatgtcccatacagcagctgtaggtcagctgctgcagctctgctccaggctgyagctctccttctgagacccaccctgaaggaacagcactgatttgggatctgctgttcttgtggcagacagaaggagcaagagggctgtcaggaattcatgatgcctctcaaagcttctgccctgaattgatattctgccacttctgctccttgggcgcaatcaagtcaggtggccaagcctgacactgggtggggaagtatattgcctacagagagatactgctgtcaatcatctggcaatgggtgggaatgggatgtaaaatcctcttacagagaggaagtgaatagctgagaggatt

SEQ ID NO: 147 - DNA, 599 bp, Homo sapiens
ctctgatggtaaagtgtcaacttgattggattgaaggatgcctagatagctgggaaagtattgtttctgggcgtgtctaggagggtgttgccagaggagattcacatttgagtcagtggactgggagaggaagcctcccctcaatgtgggtgagcaccatccagttggctgccagtgtggcttgaacaaagcaggcagaaaaaggtgggataggatgtcttgctgagtcttctagctttcatcattctcctgggctggatgcttcctgcccctggacatcagactccaggttcttcttgyacttacatgagtggtttgccaggggctctcgggcctttggccacatactgaaggctgcactgttggtttccctacttttgaggcttttggactcagactgagccactcctggcttcctacctcctcagcttgcagacaacctatcgtgggacttcaccttgtggccgtgtgagccaattctcctaataaactccctttcatagatatgtagatcctatcagttttgtccctctagagaaccttgaccaatataatagctgactgtgtcaacactgagtgagcagccactgtcactgtac

SEQ ID NO: 148 - DNA, 599 bp, Homo sapiens
tgtattgtttatagtgaatctactgcttttaaaattaaacattagatgttatatcaataatgttcacacataaacatgcttgaggcagttcccatggtggtatgtttatgtgacatcaggaacggaatttgagctttccaattccaaagggattcctttaggcccatcatcaatataaatgataccattgtttgaaatatttgcaaaatagaaaaaattaaaacctcattagaaagtgtcttactgattttttttattaccttagttgtcagggagacttctagaccttaatagctaccktcttttctataatttaatgagcagtcacacaaacaccaatattgctacagaaagggactttccagctttgggaatatctgtttctgacatttcctttcaaagggggatttttataaacataatttttcttcttccaaaagaggaaccacactatgattccaacttccatggagcccatcggggatggtggtatttcagttcaattagggtgctgtctttgttaactgttgtgtttagttgtgatctcagacttcagatttgaaatctctcttcggggaattcatttcaaaagatccaga

SEQ ID NO: 149 - DNA, 599 bp, Homo sapiens
cagggccagcccacggaaaacacaaagcccttttttccctagctagcctctccatgtggaggaacaagtcctattgcttggtggagagccaaagccctttcctttctcacttactcttctctcactgctactgggatcctggcaaaccacacaggcaggtccatgtggcttatgacacaggagttccgagctttgcgacatgacattggcaactgctcagttatccgtgattagttatgaatctaaatgcgtgggcttcccccttttccttctcaaaggctgagcttccccttgcccttyaggagcaaaagtcttcccctttctactccagcactccttggtaggcagagaagcatttacttcttccctgcctaccctgagccccggcagtattacaaacaaaagtaaaaagtaaaacacaaggcaaaactcatcagccacacgggcttgttttattccattaatattggttgttctcttttctgggagttgtgtaggcatctaatttgattattttagttatgtgtccctctctaatagaaaagaggagtgagtttgcctgtcctttttccgtttcaagcgtctgctggggaagggcc

SEQ ID NO: 150 - DNA, 599 bp, Homo sapiens
gctcactcccattgtttacttccacctggggctctgggtgcagaaacgtgagtcctcccaggtttaggagggagaaatacatttgccagcagggtttaccatgggcagggggaaggggatggtgttagtagctgcttgtctagccttgtgatttcgtctggttattctcaataggagacttcttcatcagttgccgtatttcacaaggaaagatgacaccaataagaaagaaaacgtatttgctcttgagtataatactgtgatttattcagtgaatgcatcatcactagttccctaaargctctgctctctagaaacttttcatctctaagaagtctgtgtgaataaaatgttatttgaggtctttgggctcaccgattgggggaaccagaaaccaccttcaaaagttaaagtacccattggtcttacagacaagcatcaggaaccagagggcctggtggggctgggaggaagctcggcagtgacagctgaggtgctcatgtccttccatcccactgcccagtggataatgagctcattagtcagacgaggaccagcccagaatagccaggagtaagcatgtcacattacagagctgt

SEQ ID NO: 151 - DNA, 599 bp, Homo sapiens
ttcttctcctctctccactctgttgacaggatagcttcatcttaagacttgttgtctcatcagtgtaagatgactgctaagttgtatgtgagcccacaatttcctcattcattccttgaaggaagaaaaaagatttcttcaggaacttctgacttaaaagcaatgaaggactttcccagaagtccatttctcattggcccgaattgggttgtatgcccatttctcattggttcaaattgggtcacgtgcccagtcactgacatggaggatacgatttccctaaactaatcaggcctcccrctacaactagaaggagactcagctcccacaaacatatgaattgatggtgcgggagagtgtagtgggaattctgttaggaaagagaaggaagtcaacaatcgggtacgctgtgtctaatttaagtccatgactttaaaagtgagcttttttttttgttacaaaaataatgtaggcttaattttttttttttttttgagacggagtctccccgacacccaggctggagtgcaatggcacgatctcggctcactgcaactccgcctcccaggttcaaacgattctcctgcctcagcctcccg

SEQ ID NO: 152 - DNA, 599 bp, Homo sapiens
tgtgtgactttccttggagcaaagataggcataagatatcaagctttggttgatctattgaatttactgtttagaatctgtgattgacaaggtactatcagtgtgtgtatgctctaagtcaaattcctgtgattactggtagagagactttagctcaaagctttggggaaaatgtgcctagattttcattcaatctgggccaaatctggagttgtacaaatagatagcatcggtcttgtccttagtagccagcatttttgtttaataggaggagagaaacccaggacgacactgaggaayagatatgaccagggcacttgacttcttgtttcagcactcagtcattacacaaacagttgttgaacaccaggcactttgcttggtgtgacaatgcactcccgccttccaagagctccagacccacaggggagtgctgttgtacaggtgctaggatagaggtccggggagtagaggcagaatttgggagccatcagttctatctgaggctaggaaaggcttcccaggggaggtaaaatcaggactgaggaggtcaaagagccctccatgcagaggtaagagactgacaagcacaggaaact

SEQ ID NO: 153 - DNA, 599 bp, Homo sapiens
ctagattgggcacaatatctatttcattcataaatgatcctagcaattgagaaaagagaatcatctacagaaccaatctcttacttcgaagtttttatgctgattaagcagaaaatggagtgacatcacgaaagactgtgattccattgcacatttcctaagaacaagtggctccctatacttgattgtggatttaggtgttttcaagtcctttggctgccaaacagatgtggggtaagattaccttctggtaagaacgcagtgacaaggcatgccacccctgccactatattgcttgcygcaaaggggaggcgtcgtccaaggcgctcaatggttagtaagatcaagagagctcctggcagttccaccacgcccgagatgaaaaagtctatatagaggttgccccctataattcccaggcgcatgacaagtccttgatacaccactgcgcttgtgaacctgtagcaacattgaaagacaccagagaacaagtaagagataacttcaagctgatttgaaggaagggatgtattgtaaatttaacgcaagcttttaaatccatcacaagtcaaacttaccaagcaaacataagaataagt

SEQ ID NO: 154 - DNA, 599 bp, Homo sapiens
ttcccaatgtagccactgtggtcctcaaccatgctattcctgcaaacacagaaagtttgaaagggaggagttaggggtctgatttcattaacattttacagaacaacaaaaagtatttgcttagtggcctccagtctgaagtctttgccaattttaagcacaaatgatagaactttctaataatgtaaccaaatttcaacaacaatgaaactagatgatagctaatattttagagcgctagatgcctttctgagcaggttggattataggaagtacaattcaagaaccgtttgttgagcrtgagcagccactacattggaacattgttccaggcaatgacctcctagagagccaaagcacactggctttttgtccaggtggtagtttccagtcatgcctttgagccaccacgttcccacagctggtgcccagctgtcctttctgtgcataccttgcggtggtgacacaaggaatagccgttcccagcacacctggggagttcctcacgtgagctgaccttggtttctagagtggagcaagctttgaggaccaggatctttggaaggggacatctattctgtgtgttaccactaaggcaa

SEQ ID NO: 155 - DNA, 599 bp, Homo sapiens
ttgcactccagcctgggcaacagggtaagaacctatctctaaaataaataaataataaaaaataacaaaaataagcctaatgtctaattgtatttctggtgttctctctctatggatatcatatcttcattggcccgatgctaaaaatccatgtggagggagcaggtgacacaggatcccgggcacacattccatcacctcctccaggtccctcgagtgcacctctcctcctacctccagcttccataggacttgtgaaggaacagaatcatagtctaggagggaccttggggaattcastgtcccacttctccagggctgctccaacggtcctgcctgggtttcacacacttcccctgtgccctcttctgcagtaaaggatacgggttggcccctttctcacgcaaccgtttctctatctccacttccttacttctggtagagaatgtttttcatttatggttcagcagttgaaatttgaatgaaaatactgagcccatgaggacagaccttctgcttcatgcaaaagtcctttgacagtgtgagggacaaagagtcggcttctaccacttgacaccagtgacctcaatacaggatca

SEQ ID NO: 156 - DNA, 599 bp, Homo sapiens
ggaggaatcggagggagaggggcaggtaagtgagagtgtcagtgagcgggcgcagaggggactcccctccagcacttctgtttccctgtgatgcaggagaggaggccttgggtgttggaccctaagaaggaactcagtcaatcactgtgagatgtgtgagtgaacacatggctgccaggtatacaggggccgtggtgctactcagggtttgaggaagggagagaacctttgaagctgtggtaagggagagctggggcattgatctgggatgcagaggttgctgtggttgagagctactcyagtgagcaacatgatggcttcagagtgagcaggccccatgggagagggcccagctgtgtcttcctggagcggtaacacctttcccctattccataggtatcctggcatccatctgtggtggccttgtgatgcttttgcctgaaaccaagggtattgccttgccagagacagtggatgatgtagaaaaacttggcaggtactgtacaaaattcaatgcaccctaaataaaagcaatatttaaaaccacgggggaaagaatgacattgagaaaattgaatatccacagggaaaaaaataag

SEQ ID NO: 157 - DNA, 599 bp, Homo sapiens
gagaggggcaggtaagtgagagtgtcagtgagcgggcgcagaggggactcccctccagcacttctgtttccctgtgatgcaggagaggaggccttgggtgttggaccctaagaaggaactcagtcaatcactgtgagatgtgtgagtgaacacatggctgccaggtatacaggggccgtggtgctactcagggtttgaggaagggagagaacctttgaagctgtggtaagggagagctggggcattgatctgggatgcagaggttgctgtggttgagagctactccagtgagcaacatgrtggcttcagagtgagcaggccccatgggagagggcccagctgtgtcttcctggagcggtaacacctttcccctattccataggtatcctggcatccatctgtggtggccttgtgatgcttttgcctgaaaccaagggtattgccttgccagagacagtggatgatgtagaaaaacttggcaggtactgtacaaaattcaatgcaccctaaataaaagcaatatttaaaaccacgggggaaagaatgacattgagaaaattgaatatccacagggaaaaaaataagaataaatcctcatc

SEQ ID NO: 158 - DNA, 599 bp, Homo sapiens
tcccacattcttgatggagctgtcatgagaatcaggttgggggcttctcttgtctctctttagcctaatgggtcagagaccacagggaacacaagaaaaaaacccaggaagggctgtgcacctgacagcttctttgttaaactggctacatattatgtgttcccagaaacttcaacagcattacaaatcctaactcatccaacattatataaaaatactctggatagattgaggtctttatacagtattacaacagcgcggcatgatgtagctgaaatgcacacgtgcatctctgcaagrcacagctttgcaaggaggatcagctcctggatagctccttctttctgtctttgtcgggggcctcaaaggtgagagcgggaaactggggttttcttattcctgccacatttacaggaatgtggactgaaaaaacaaaggagaaagtctttaggaaagattatgtaactagttagaagagggtgttctgttattatttaatagtatagctggttttgatcaaatacattcatgaaaacccaactctaaggtaacaacaatatctgtctccaggatcattttttgtctgtgttcagacctta

SEQ ID NO: 159 - DNA, 599 bp, Homo sapiens
tttcttgtgttccctgtggtctctgacccattaggctaaagagagacaagagaagcccccaacctgattctcatgacagctccatcaagaatgtgggatgtgccgaccaaggatttgagaaagttgtacagaaatgtgttcatcaaatctggtcaagggactaagctcctagctgaccattcattctgaagattgcatggaggatgaacatctgggaatcctgttaatgagaaggctgaatcacaggcacctgggccaaagggtgtgagcattcatgttctctgctcaccttggtttccrcacaccttcgcaatgtgaacaggtcaggagtccctcccgtccacctcctctgtaacagctggggttccaggcatggtttaggccctgttccagcaataagaaccaatctgctgtacaatctgaggacttggctctgttatttacaaaatgatgctgtggttctgagattatttgggacatttttggctctcctttagtggacacctagagccacagattcccttctttactaaacaaatcccatggattctgatttctgggtcttaggattttaaaagtgaagggatatttttcttata

SEQ ID NO: 160 - DNA, 599 bp, Homo sapiens
ccacccacattcctggccctggcaagtcagtcttacaacaaggtaagaacaggcccagaaaccaactatattgtccttccacacaagccccacaaaatccttctacatttacacagaacccacacagctgaggcatcagtacctgcctctatgttttctgaaggaggaaaaaatatagaaaaataaaaataattatattattttaggttctctactttgaaaatggccaaaattggaatctttttcttgatctaagtagctacatcaaaatctgtaggcgcagtgatttgcaccaagttycgatattgctggaaaatattgaagatgctctgagggtttctatggatatccattgtctcactgtcagatgaaaagagagggaagtttttagaaatgtgacactttgcagtgagggaggacaagagcaaacttacctacagtctgtcacaggcacgattttttttttacacttttgtgaatcattgaattcaatgccgaggctactcatccattcacaaacacaggaaaaaattataggtcatgatccccataaatgaagagtaatcagtccaatcccacagaacctggaccttttgggt

SEQ ID NO: 161 - DNA, 599 bp, Homo sapiens
tttaatagctagttcatttccactaagacattcagtataaataataagtaaaaataaatagaaacattcagttttattttaaatagtaggagcaggtaccataatttctgtagttactcttttagtacaatgatgcatgtatactgtgtgtaaggcatattagcagaaattgagttcagcactagggaagatgatttcattccatgcaatgtttctttaaaaaaaaaaaagacttatatagatagattctcagaacagcccatggcaaatggacagttatttggaaaactcaggttccarattcactggagtgtagaatctctggttggttggggaggaaattcctcttgcagttgttattaataattatgtgaatattattaactatattaatgtttataattttgaagaccttgaaggactggagacagagaagcatttttgaacaccctctgtagcctatgcactgttgtaggcattgatgggtggtaccaaagatgggacactgttcctacctccagagatcttgcaggctggctgcagagagaaggcagagaggaggtaaaagaaaaataaaggcacatgtgtgtaagtcaccc

SEQ ID NO: 162 - DNA, 599 bp, Homo sapiens
tatttctgaaaaaacaaaaagccacttggacttgtcccctattttactttctgctgaccaatcaacttgtctgtcctagttacagaaagctgtcctggacttttcatttgtataatttggttgcttgtggttgggtctgccatgtggaagaaccttgagctgggggaagcagcttggcctccaagtccactgaagaccaccattccaagatttcctggggaggcggtacagggcagtgatgaagattatgggagccacactgcccagcttcgcatttgggacactcctggggagagcaaragggaagaatgagaggttagggtgatagtaaagattctacttggccaatattattgtattgtggcattgtggtctctggatttagtgtgagttgatagctgaccttttccttgagtgggtgtttttgtttgttctgcagaagcatcttggtggctcctctacctgaaagtccctcgatggggtcagttccagttctccacttctggccccgtctggtacatgccactgcctctcactgcctgggctctctatccttgacaggctgccttgaagttgagcccacactgactttcttgcct

SEQ ID NO: 163 - DNA, 599 bp, Homo sapiens
acacatgtgacccagtctcaagcccagactcccagctacaggaggctggttctccaagcaactggatatgttagcaagtgtggtattcgaaggagatttttttatctattttcaactgaggtacactttcatgtatagtcaaatgcacagatcttagatgtagtttgatgaattttgatgaacatgtacacttgtgtaactcgcaagttacgatcgtgacacagaacatttacatcaccccagaaacaacttttgttttacggcaaaatcaatctctgctaatttcttcccatggccagwggcaaatgatactggtttccttcaagagaaattatttttgcctttctggaaccttatatcaatggactcatatataatgttcatttgtgttatctgacatctttccttaatgtaatatttttgagcttcatccatgctatttgagagtatcattaatgtgctcccttttattgctgaatagtatgccattgtatgaatagatcgcaaattatttattcattttcttattgattgacactgtagttgcctacagtttggggtataaaaaataaagctgctatgaatattcttgtagatct

SEQ ID NO: 164 - DNA, 599 bp, Homo sapiens
aagaattaaaagatctaaataaagatacaccatgcttatagattgaaaggctcaatatcacttctcatcaaattgatctttggattaaactctcacccaaacactattgctgcagtccttttttggaaaaaatgtggaggacttacataccttaatataaagacttataaaggtacaagaatcaagacgtggtattggccaggacccttggctcatgcttgtaaccccaacactttgggaggctgaggcaggaggatggcttgagcccaggtgttcaagaccagcctcagcaacagagcragaccctctctccacaaaaaaaaatgaaccattagatgggtgtgatgagtcacacctgtagttccagccactcaggacactgaggggagaggatcacttgacccaggaggtctaggcatgagtgaaccttgatcatgccactacattatagcctggatgacagaatgataccctatctcaaaaaataaaaagatatgcgttattggcaaaaaagtatgcaaaactaaaaagggatggtccagcaccagacccacatacatatggtcaattgattttctatatagatgacaaagcaatta

SEQ ID NO: 165 - DNA, 599 bp, Homo sapiens
actctttgtgctcaaaatgtagcaaaaaggctctatgttattatcatggaaaaccatcatgcaaatagtgttactgtctgtcgataccgtatcaggtgggtcagaaaggtttgtttctgcaagacttattaaagcttccgtgcaaaactagcttccaggcagaatgcagtatctctaggatgggtcccagtacatgacctcctctcctgctctcagtccatcttctactggccgcagccactccgggactgagggaatgggggatgagttgaaggaacagtgggtcccaaggcataaacrgagatggatgaatagactatgagatctgaagaggttaggcagctatggagaagttagtagttggtcatttgtggtcatgaccatttttttcatgtctttggtagccacttatggcagagagacttgccattggatggtgggaccccagcttctgtgcatctatctaaccccctcgcttacttttcagtctctgtctctctcctgagtctcatctgtctttttgcctttgttcgtctcttcttggaaacctggacctattcagaccactggtgctcaggacccagtgtgtttctgctgat

SEQ ID NO: 166 - DNA, 599 bp, Homo sapiens
aggaaacacacaaagtttccatgtctgtccccacctctgacctcaacagcctatcgccacctaaagcctgcactgccaaatccatcacactttgtactctcatgccccattgtctccatgaacattcagtagagatccctagagatgagcttgcacgtgagagcagtttcccatgaaaaggactctgaactgttgtttttctctaagcttttatatgaaatatttagaagaacttactacatttccagaatggtaggaatctgttgcttttgtgtttgtttgttttctcatgtccatctrcttacaaataaaaaaaagagaatgattgtaattcttgaagactcatttctggtttgtggcattgatagcttcatctgtttccctgtcctaggaaatactcaccttgaatttttctgttttaattcagcactgattgatccaaggccttcaatgtaggagtgctaccatggaaatggacagagttatcgaggcacatacttcaccaccgtcacaggaagcacttgccaagcttggtcatctatgacaccacaccagcacagtaggacctcagaaaagtacccaaatgcgtacgtctttgt

SEQ ID NO: 167 - DNA, 599 bp, Homo sapiens
ttttgtagctatttagagcagagagactggccatgagttggtcagacccggcttctgtccatgtacccaccccttttatttttcagcatctgtctctttgctgtctctcatcagccttcctgcctttgcccttttctttctggcacctcatatcttttcagaccattgatgcttgggatcgagtgtgtttctacccattggaatagatgtagaaatagaaagagattcgactctccatagatatgcacatactccttcctcgtgagccaagacagaaaaggccaacacaacttttacttygggaatcagaggttagaatgatagaattccaaggtggaaaggctgcattgactcttactgtgtgttgtagaaaacactctaagtgtttggtcatttgtggtcctgacagttttcgtaatgtgatcacctcgccttcagatagcctcctgatgcataaagctatgaggaaggagagccagattaacatttcttatctctgagaccctttgcttcaaggcaatattcctgagacattttgctatgccctctacatctgacacaatttgattcctgagcacttagctagaagcacgtctctt

SEQ ID NO: 168 - DNA, 599 bp, Homo sapiens
atgcttcactctgtctcccgtccaattaattatttctcatcattccctcaatccaagtaacaaaccttgaaacacgagcatagacaccaggcttattggggcgtgcacagccaagaccccaagaagtgactccttgtaaaatgtatttgtccttctcgaagcaaaccagaggccctccactgtcaccctaaacagaggtaggggaaaattcatgtgagctttaagctgccaaccttttaatatgggatgccatccttctcttatccatatgtacattttcccagtaattcaaatcagaarggaacttatgagcatattactcaagcatctgcagtgcttaagtacttagcaaagtctattcaacaaaaccacatcacttagttttttcctgggaaaaaaaataatcccttgtgaatcctgcatgcattgcttcctaacttggtgtttcctttagactgggctagcctggacatcattgttaagtgtgtgtaatttgtctcggtcctgtaccctctaccttcctctctactggaaaagccaccacttcagtcactggtgaccagcacacaccaggaaggtgagtggcctgaggtagttat

SEQ ID NO: 169 - DNA, 599 bp, Homo sapiens
tcctggaaatatggtgacggttgcacaccattgtgaatgtactgaatgccactcagtggtacctgcaaaaatggttaaaatagtaactctatgttgtataaattttattgcaataaaaaagaatagtcatcatttactaagaatcaacctttccaggcacaagctcataatgtggtatatctcacttaatcctcacaacggtcctatgaaacaggcacttgtcattcctgttcttcagttgaggaaactgaggcgtaaggaggttgtatagctcagcttgagtctgggtctgcggagtcrctgcccaaatgcttcctccccatgcgtgttggccccactcttgcttccaaccttccacatgtttgttcataaatttcattcttattccttattactttctgaagtctcagttcccataattatatagacatatttctatctcacagtaacattgccaaattattctctaaaagggtgtgacagtggaaaatgtcactggaaaacaattactttgcctcacccttgtgatattcaatttttaaaatattagctaacatagtaggtggaaattgtcctgattcattttctaatttacactt

SEQ ID NO: 170 - DNA, 599 bp, Homo sapiens
aacactgtgtccaggaaggaagacaggttaatgatgaagttgatctcctttgattccctttctcagttctctcaagcctaccatatctgttgtccaacctgtgaaaactgttttctcatgtatttgatagtccttcatggcagaaaggctggcatagccagacatgggtttctgtccatcttcctgaatgtgtaactcttcagcatctctatacttcctgactctcagctgccttcctccttttgcccctcatgggctggcaacacatagcttttcagacaccttgttctcagaacccartgtgtttatacaggttagaggagaaagtgatcggaagatctcgaagtcaatatagatatccttcacttgttacccaagacaaacatggcaaacacaaccttcactccagggcttggagattagaatgacagaattccctggttgaactatttcactggtctttagtgggcattgtggaatggtgctatttctaagcacatggtcatatgttcttctgagctttctattcatgagacctcaccacctccaagtatcctcctacttctatcaactatgagaaaagagagctggcctgacat

SEQ ID NO: 171 - DNA, 599 bp, Homo sapiens
tcttctcagcctctagtaccttctgtcctactttttacttcctgtgttcaactcttttagctgacacatatgagtgagaacatgcagtgctaacctttctgttcctggcttatttaacataatgtcctctaacgtcactcatgttactgtgaatgataggattccattctttttatggctgaatagtattccatgatatgtatataccacattttctttatccattcatgtgtggtcagacaactaggttgattccatgtcttggttattgtgaagagagctgcaataaacctggggctkcagatgactctttgatataatgatttccattctgttagataaagcccagtagtgagatagctggatcatatggtacttctatttgtagttttttaaggaacctgcatactgttctccagagtgattgcgcagtttaaattcctactaactatgtacaaaagttccctttttttctgcatcctcaccagcatgctatttttgtctttttgataatagccatccttactggggtgagaaaatagctcattgtggtttaggtttgcatttctctgaggattagtgatgttgagcacattttc

SEQ ID NO: 172 - DNA, 599 bp, Homo sapiens
gggcttcctcaggagaagagatggtctcattgctcagggaaccttttctcctaggcaaacagaggtagtgtggcagctggaagaggcctggagtggtacttggactttgaagccaaacacttgccctctcagagcctttgcctccactgtggtcctaccagagagctgagttcaggacctgggtttgggcgtggaagggcggtgtgagagcaaggctgaatagtccaacttcctcctttaacaaatatccactgagcgtcttcgctacgcaaggctctcttccagaccgcttggttcttwggagtccaggagttctccactcggtacacccagttttaccaaagttaaacccatgagattttgtaataaagccaaggctaataataatgcattccattacttttcctacacatgtgtctctgttaagcactttgcaaggaggatgggcagctgggccaatgcaggatccaactattcaagattcctccacttacacaatatattgacactaactctgaatgcatccctactgaatagagaactctagtaatttctactgaaaacatcaaataaaaaagctactactttaatagaaatta